SEQUENCE LISTING (455 sequences)

SEQ ID NO: 1 - protein, 121 aa, synthetic construct
  REGION 1..121: note = Synthetic
EVQLVQSGAEVKKPGSSVKVSCKASGYTFTNYYMHWVRQAPGQGLEWMGAIMPSRGGTSYPQKFQGRVTMTGDTSTSTVYMELSSLRSEDTAVYYCARGEYYYDSSGYYYWGQGTLVTVSS

SEQ ID NO: 2 - protein, 119 aa, synthetic construct
  REGION 1..119: note = Synthetic
EVQLVQSGTEVKKPGASVKVSCKASGYTFTTYAMHWVRQAPGQSLEWMGWINTGNGNTKYSQNFQGRVTMTRDTSISTAYMELSRLRSDDTAVYYCARDLGQLERLYFWGQGTLVTVSS

SEQ ID NO: 3 - protein, 114 aa, synthetic construct
  REGION 1..114: note = Synthetic
HVQLVETGGGLVQPGGSLRLSCAASGFTFSSYAMSWVRQAPGKGLEWVSAISGSGGSTYYADSVKGRFTISRDNSKNTLYLQMNSLRAEDTAVYYCARDLGDYWGQGTLVTVSS

SEQ ID NO: 4 - protein, 117 aa, synthetic construct
  REGION 1..117: note = Synthetic
QVQLQQWGAGLLKPSETLSLTCAVYGGSFSGYYWSWIRQPPGKGLEWIGEINHSGSTNYNPSLKSRVTISVDTSKNQFSLKLSSVTAADTAVYYCARSSSGDAFDIWGQGTMVTVSS

SEQ ID NO: 5 - protein, 121 aa, synthetic construct
  REGION 1..121: note = Synthetic
EVQLVQSGAEVKKPGSSVKVSCKASGYTFTNYYMHWVRQAPGQGLEWMGAIMPSRGGTSYPQKFQGRVTMTGDTSTSTVYMELSSLRSEDTAVYYCARGEYYYDSSGYYNWGQGTLVTVSS

SEQ ID NO: 6 - protein, 121 aa, synthetic construct
  REGION 1..121: note = Synthetic
EVQLVQSGAEVKKPGSSVKVSCKASGYTFTNYYIHWVRQAPGQGLEWMGAIMPSRGGTSYPQKFQGRVTMTGDTSTSTVYMELSSLRSEDTAVYYCARGEYYYDSSGYYYWGQGTLVTVSS

SEQ ID NO: 7 - protein, 121 aa, synthetic construct
  REGION 1..121: note = Synthetic
EVQLVQSGAEVKKPGSSVKVSCKASGYTFTNYYMHWVRQPPGQGLEWMGAIMPSRGGTSYPQKFQGRVTMTGDTSTSTVYMELSSLRSEDTAVYYCARGEYYYDSSGYYYWGQGTLVTVSS

SEQ ID NO: 8 - protein, 121 aa, synthetic construct
  REGION 1..121: note = Synthetic
EVQLVQSGAEVKKPGSTVKVSCKASGYTFTNYYMHWVRQAPGQGLEWMGAIMPSRGGTSYPQKFQGRVTMTGDTSTSTVYMELSSLRSEDTAVYYCARGEYYYDSSGYYYWGQGTLVTVSS

SEQ ID NO: 9 - protein, 121 aa, synthetic construct
  REGION 1..121: note = Synthetic
EVQLVQSGAEVKKPGSSVKVSCKASGYTFTNYYMHWVRQAPGQGLEWMGAIMPSRGGTSYPQKFQGRVTMTGDTSTSTVYMELSSLRSEDTAVYYCARGEYYYDSSGYYYWGQGTPVTVSS

SEQ ID NO: 10 - protein, 121 aa, synthetic construct
  REGION 1..121: note = Synthetic
EVQLVQSGAEVKKPGSSVKVSCKASGYTFTNYYMHWVRQAPGQGLEWMGAIMPSRGGTSYPQKFQGRVTMTGDTSTSTVYMELSNLRSEDTAVYYCARGEYYYDSSGYYYWGQGTLVTVSN

SEQ ID NO: 11 - protein, 121 aa, synthetic construct
  REGION 1..121: note = Synthetic
EVQLVQSGAEVKEPGSSVKVSCKASGYTFTNYYMHWVRQAPGQGLEWMGAIMPSRGGTSYPQKFQGRVTMTGDISTSTVYMELSSLRSEDTAVYYCARGEYYYDSSGYYYWGQGTLVTVSS

SEQ ID NO: 12 - protein, 121 aa, synthetic construct
  REGION 1..121: note = Synthetic
EVQLVQSGAEVKKPGSSVKVSCKASGYTFTNYYIHWVRQAPGQGLEWMGAIMPSRGGTSYPQKFQGRVTMTGDTSTSTVYMELNSLRSEDTAVYYCARGEYYYDSSGYYYWGQGTLVTVSS

SEQ ID NO: 13 - protein, 121 aa, synthetic construct
  REGION 1..121: note = Synthetic
EVQLVQSGAEVKKPGSSVKVSCKASGYTFTNYYMHWVRQAPGQGLEWMGAIMPSRGGTSYPQKFQGRVTMTGDTSTSTVYMELSSLRSEDTAVYYCARGEYYYDSSGYYYWGPGTLVTVSS

SEQ ID NO: 14 - protein, 121 aa, synthetic construct
  REGION 1..121: note = Synthetic
EVQLVQSGAEVKKPGSSVKVSCKASGYTFTNYYMHWVRQAPGQGLEWMGAIMPSRGGTSYPQKFQGRVTMTGDTSTSTVYMELSSLRSEDTAMYYCARGEYYYDSSGYYYWGQGTLVTVSS

SEQ ID NO: 15 - protein, 121 aa, synthetic construct
  REGION 1..121: note = Synthetic
EVQLVQSGAEVKKPGSSVKVSCKASGYTFTNYYMHWVRQAPGQGLEWMGAIMPSRGGTSYPQKFQGRVTMTGDTSTSTVYMELSSLRTEDTAVYYCARGEYYYDSSGYYYWGQGTLVTVSS

SEQ ID NO: 16 - protein, 121 aa, synthetic construct
  REGION 1..121: note = Synthetic
EVQLVQSGAEVKKPGSSVKVSCKASGYTFTNYYMHWVRQAPGQGLEWMGAIMPSRGGTSYPQKFQGRVTMTGDTSTSTVYMELSNLRSEDTAVYYCARGEYYYDSSGYYYWGQGTLVTVSS

SEQ ID NO: 17 - protein, 121 aa, synthetic construct
  REGION 1..121: note = Synthetic
EVQLVQSGAEVKKPGSSVKVSCKASGYTFTNYYMHWVRQAPGQGLEWMGAIMPSRGGTSYPQKFQGRVTMTGDTSTSTVYMELSSLRSEDTAVYYCARGEYYYDSSGYYYWGQGTLVNVSS

SEQ ID NO: 18 - protein, 121 aa, synthetic construct
  REGION 1..121: note = Synthetic
EVQLVQSGAEVKKPGSSVKVSCKASGYTFSNYYMHWVRQAPGQGLEWIGAIMPSRGGTSYPQKFQGRVTMTGDTSTSTVYMELSSLRSEDTAVYYCARGEYYYDSSGYYYWGQGTLVTVSS

SEQ ID NO: 19 - protein, 121 aa, synthetic construct
  REGION 1..121: note = Synthetic
EVQLVQSGAEVKKPGSSVKVSCKATGYTFTNYYMHWVRQAPGQGLEWMGAIMPSRGGTSYPQKFQGRVTMTGDTSTSTVYMELSSLRSEDTAVYYCARGEYYYDSSGYYYWGQGTLVTVSS

SEQ ID NO: 20 - protein, 121 aa, synthetic construct; identical to SEQ ID NO: 9
  REGION 1..121: note = Synthetic

SEQ ID NO: 21 - protein, 121 aa, synthetic construct
  REGION 1..121: note = Synthetic
EVQLVQSGAEVKKPGASVKVSCKASGYTFTNYYMHWVRQAPGQGLEWMGAIMPSRGGTSYPQKFQGRVTITADKSTSTAYMELSSLRSEDTAVYYCARGEYYYDSSGYYYWGQGTLVTVSS

SEQ ID NO: 22 - protein, 126 aa, synthetic construct
  REGION 1..126: note = Synthetic
EVQLVQSGGGVVQPGRSLRLSCAASGFTFSSYAMHWVRQAPGKGLEWVAVISYDGSNKYYADSVKGRFTISRDNSKNTLYLQMNSLRAEDTAVYYCARDLGYSSSWYYYYYGMDVWGQGTTVTVSS

SEQ ID NO: 23 - protein, 120 aa, synthetic construct
  REGION 1..120: note = Synthetic
QVQLQESGPGLVKPSETLSLTCTVSGVSISSRSDHWGWVRQPPGKGLEWIGSISYSGSTYYNPSLKSRVTISVDTSKNQLSLKLSSVTAADTAVYYCARESHPAAALVGWGQGTLVTVSS

SEQ ID NO: 24 - protein, 112 aa, synthetic construct
  REGION 1..112: note = Synthetic
EVQLVQSGGGLVQPGGSLRLSCAASGFTFSSYAMSWVRQAPGKGLEWVSAISGSGGSTHYADSVKGRFTISRDNSKNTLYLQMNSLRAEDTAVYYCATPAFWGQGTLVTVSS

SEQ ID NO: 25 - protein, 117 aa, synthetic construct
  REGION 1..117: note = Synthetic
QVQLQQWGPGLVKPSETLSLTCTVSGGSISSYYWSWIRQPPGKGLEWIGEINHSGSTNYNPSLKSRVTISVDTSKNQFSLKLSSVTAADTAVYYCAGGSNLDWFDPWGQGTLVTVSS

SEQ ID NO: 26 - protein, 118 aa, synthetic construct
  REGION 1..118: note = Synthetic
QVQLQQWGAGLLKPSETLSLTCAVYGGSFSGYYWSWIRQPPGKGLEWIGEINHSGSTNYNPSLKSRVTISVDTSKNQFSLELSSVTAADTAVYYCARADRRFGELRYWGQGTLVTVSS

SEQ ID NO: 27 - protein, 121 aa, synthetic construct
  REGION 1..121: note = Synthetic
QVQLVQSGGGLVKPGGSLRLSCAASGFTFSSYDLHWVRQVPGKGLEWVSLISYDGSNKYYADSVKGRFTISRDNAENSLYLQMNSLRAEDTAVYYCAREPITGTSDLFDYWGQGTLVTVSS

SEQ ID NO: 28 - protein, 117 aa, synthetic construct
  REGION 1..117: note = Synthetic
QVQLQESGPGLVKPSGTLSLTCAVSGGSISSSNWWSWVRQPPGKGLEWIGEIYHSGSTNYNPSLKSRVTISVDKSKNQFSLKLSSVTAADTAVYYCVREGGLREEHWGQGTLVTVSS

SEQ ID NO: 29 - protein, 117 aa, synthetic construct
  REGION 1..117: note = Synthetic
QVQLQQWGAGLLKPSETLSLTCAVYGGSFSGYYWSWIRQPPGKGLEWIGEINHSGSTNYNPSLKSRVTISVDTSKNQFSLKLSSVTAADTAVYYCARGTDTAMADYWGQGTLVTVSS

SEQ ID NO: 30 - protein, 120 aa, synthetic construct
  REGION 1..120: note = Synthetic
QVQLQQSGPGLVKPSQTLSLTCAISGDSVSGNSATWNWIRQSPSRGLEWLGRTYYRSKWNHDYAESVKSRITINPDTSKNQFSLQLNSVTPEDTAVYYCARDSKSAFDIWGQGTMVTVSS

SEQ ID NO: 31 - protein, 113 aa, synthetic construct
  REGION 1..113: note = Synthetic
QLQLQESGPGLVKPSETLSLTCSVFGVSITSGSWWSWVRQSPGKELEWIGEIYHNGNTNYNPSLKSRVTISVDTSKNQFSLKLSSVTAADTAVYYCVSGFDYWGQGTLVTVSS

SEQ ID NO: 32 - protein, 120 aa, synthetic construct; identical to SEQ ID NO: 23
  REGION 1..120: note = Synthetic

SEQ ID NO: 33 - protein, 126 aa, synthetic construct
  REGION 1..126: note = Synthetic
EVQLVQSGAEVKKPGSSVKVSCKASGYTFTSYGISWVRQAPGQGLEWMGWISAYNGNTNYAQKLQGRVTMTTDTSTSTAYMELSSLRSEDTAVYYCARAPDYGDSSNYYYYYMDVWGKGTTVTVSS

SEQ ID NO: 34 - protein, 127 aa, synthetic construct
  REGION 1..127: note = Synthetic
QVQLVQSGAEVKKPGASVKVSCKASGYTFTSYGISWVRQAPGQGLEWMGWISAYNGNTNYAQKLQGRVTMTTDTSTSTAYMELRSLRSDDTAVYYCARDTSGDYSSGWYLGVPFDYWGQGTLVTVSS

SEQ ID NO: 35 - protein, 108 aa, synthetic construct
  REGION 1..108: note = Synthetic
QSALTQPASVSGSPGQSIAISCTGTSSDIGHYDFVSWYQQHPGTAPKLIIYDINNRPSGISNRFSGSKSDNMASLTISGLQPEDEADYYCSAYTSSDTLVFGGGTKLT

SEQ ID NO: 36 - protein, 107 aa, synthetic construct
  REGION 1..107: note = Synthetic
DIQMTQSPSTLSASVGDRVTLSCRAGQAISSWLAWYQQKPGKAPKLLIYKASNLESGVPSRFSGGGSGAEFTLTISSLQPDDFATYYCQQYQSYPYTFGQGTKLEIR

SEQ ID NO: 37 - protein, 107 aa, synthetic construct
  REGION 1..107: note = Synthetic
DIQLTQSPSSLSASVGDRVTITCQASQDIGNYLNWYQLKPGKAPKLLIYDASNLETGVPSRFSGSGSGTDFTFTISSLQPEDIATYYCLQLYDYPLTFGGGTKVEIK

SEQ ID NO: 38 - protein, 110 aa, synthetic construct
  REGION 1..110: note = Synthetic
NFMLTQPHSVSESPGKTVTISCTRSSGSIASNYVQWYQQRPGSSPTTVIFDDNQRPTGVPDRFSAAIDTSSSSASLTISGLTAEDEADYYCQSSHSTAVVFGGGTKLTVL

SEQ ID NO: 39 - protein, 108 aa, synthetic construct
  REGION 1..108: note = Synthetic
QSALTQPASVSGSPGQSIAISCTGTSSDIGDYDFVSWYQQHPGTAPKLIIYDINNRPSGISNRFSGSKSDNMASLTISGLQPEDEADYYCSAYTSSDTLVFGGGTKLT

SEQ ID NO: 40 - protein, 108 aa, synthetic construct
  REGION 1..108: note = Synthetic
QSALTQPASVSGSPGQSIAISCTGTSSDIGHYDFVSWYQQHPGTAPKLIIYDNNNRPSGISNRFSGSKSDNMASLTISGLQPEDEADYYCSAYTSSDTLVFGGGTKLT

SEQ ID NO: 41 - protein, 108 aa, synthetic construct
  REGION 1..108: note = Synthetic
QSALTQPASVSGSPGQSITISCTGTSSDIGHYDFVSWYQQHPGTAPKLIIYDINNRPSGISNRFSGSKSDNMASLTISGLQPEDEADYYCSAYTSSDTVVFGGGTKLT

SEQ ID NO: 42 - protein, 108 aa, synthetic construct
  REGION 1..108: note = Synthetic
QSALTQPASVSGSPGQSIAISCTGTSSDIGHYDFVSWYQQHPGTAPKLIIYDINNRPSGISNRFSGSKSDNMASLTISGLQPEDEADYYCSAYTSSDTVVFGGGTKLT

SEQ ID NO: 43 - protein, 108 aa, synthetic construct
  REGION 1..108: note = Synthetic
QSALTQPASVSGSPGQSIAISCTGTSSDIGHYDFISWYQQHPGTAPKLIIYDFNNRPSGISNRFSGSKSDNMASLTISGLQPEDEADYYCSAYTSSDTLVFGGGTKLT

SEQ ID NO: 44 - protein, 108 aa, synthetic construct
  REGION 1..108: note = Synthetic
QSALTQPASVSGSPGQSIAISCTGTSSDIGHYDFVSWYQQHPGTAPKLIIYDNNNRPSGISNRFSGSKSDNMASLTISGLQPEDEADYYCSAYTSSDTVVFGGGTKLT

SEQ ID NO: 45 - protein, 108 aa, synthetic construct
  REGION 1..108: note = Synthetic
QSALTQPASVSGSPGQSIVISCTGTSSDIGHYDFVSWYQQHPGTAPKLIIYDINNRPSGISNRFSGSKSDNMASLTISGLQPEDEADYYCSAYTSSDTVVFGGGTKLT

SEQ ID NO: 46 - protein, 108 aa, synthetic construct
  REGION 1..108: note = Synthetic
QSALTQPASVSGSPGQSIAISCTGTSSDIGDYDFVSWYQQHPGTAPKLIIYDINNRPSGISNRFSGSKSDNMASLIISGLQPEDEADYYCSAYTSSDTLVFGGGTKLT

SEQ ID NO: 47 - protein, 108 aa, synthetic construct
  REGION 1..108: note = Synthetic
QSALTQPASVSGSPGQSIAISCTGTSSDIGHYDFVSWYQQHPGTAPKLIIYDINNRPSGISNRFSGSKSDDMASLTISGLQPEDEADYYCSAYTSSDTVVFGGGTKLT

SEQ ID NO: 48 - protein, 108 aa, synthetic construct
  REGION 1..108: note = Synthetic
QSALTQPASVSGSPGQSIAISCTGTSSDIGHYDFVSWYQQHPGTAPKLIIYDINNRASGISNRFSGSKSDNMASLTISGLQPEDEADYYCSAYTSSDTVVFGGGTKLT

SEQ ID NO: 49 - protein, 108 aa, synthetic construct
  REGION 1..108: note = Synthetic
QSALTQPASVSGNPGQSIAISCTGTSSDIGHYDFVSWYQQHPGTAPKLIIYDINNRPSGISNRFSGSKSDNMASLTISGLQPEDEADYYCSAYTSSDTVVFGGGTKLT

SEQ ID NO: 50 - protein, 108 aa, synthetic construct
  REGION 1..108: note = Synthetic
QSTLTQPASVSGSPGQSITISCTGTSSDIGHYDFVSWYQQHPGTAPKLIIYDINNRPSGISNRFSGSKSDNMASLTISGLQPEDEADYYCSAYTSSDTVVFGGGTKLT

SEQ ID NO: 51 - protein, 108 aa, synthetic construct
  REGION 1..108: note = Synthetic
QSALTQPASVSGSPGQSIAISCTGTSSDIGDYDFVSWYQQHPGTAPKLIIYDINNRPSGISNRFSGSKSDNMASLTISGLQPEDEADYYCSAYTSSDTVVFGGGTKLT

SEQ ID NO: 52 - protein, 108 aa, synthetic construct
  REGION 1..108: note = Synthetic
QSVLTQPPSVSGAPGQRVTISCTGTSSDIGHYDFVSWYQQLPGTAPKLLIYDINNRPSGVPDRFSGSKSGTSASLAITGLQAEDEADYYCSAYTSSDTLVFGGGTKLT

SEQ ID NO: 53 - protein, 112 aa, synthetic construct
  REGION 1..112: note = Synthetic
DVVMTQSPLSLPVTPGEPASISCRSSQSLLHSNGYNYLDWYLQKPGQSPQLLIYLGSSRASGVPDRFSGSGSGTDFTLKISRVEAEDVGVYYCMQALQTPRTFGQGTKLEIK

SEQ ID NO: 54 - protein, 106 aa, synthetic construct
  REGION 1..106: note = Synthetic
DIQMTQSPSSLSASVGDRVTITCQASQDISDYLNWYQQKPGKAPQILIYDASNLETGVPSRFSGSGSGTDFTFTISNLQPEDVATYYCQQYEDLPSFGGGTKVEIK

SEQ ID NO: 55 - protein, 107 aa, synthetic construct
  REGION 1..107: note = Synthetic
DIQLTQSPSSLSASVGDRVTITCRASQSISSYLNWYQQKPGKAPKALIYDASNLETGVPSRFSGSGSGTDFTLTIISLQPEDFATYFCLQDYIYPWTFGQGTKVEFK

SEQ ID NO: 56 - protein, 111 aa, synthetic construct
  REGION 1..111: note = Synthetic
QSVLTQPPSVSGAPGQRVTISCTGGSSNVGAGYDVHWYQQLPGTVPKLLIYDNTNRPSGVPDRFSASKSGTSASLVITGLQAEDEGDYYCQSYDSSLRASVFGGGTMLTVL

SEQ ID NO: 57 - protein, 111 aa, synthetic construct
  REGION 1..111: note = Synthetic
NFMLTQPHSVSESPGKTVTISCTGSSGSIASSYVQWYQQRPGSAPTTVIYADNQRPSGVPDRFSGSVDSSSNSASLTISGLKTEDEADYYCQSFDSSLYMIFGGGTKLTVL

SEQ ID NO: 58 - protein, 111 aa, synthetic construct
  REGION 1..111: note = Synthetic
QSVLTQPPSVSGAPGQRVTISCTGSRSNIGAGYDVHWYQHLPGTAPKLLIYDNSNRPSGVSDRFSGSKSGTSASLAITGLQAEDEADYYCQSFDSSLRGVVFGGGTRLTVL

SEQ ID NO: 59 - protein, 111 aa, synthetic construct
  REGION 1..111: note = Synthetic
SYELTQPPSASGTPGQRVTISCSGSSSNIGSNTVNWYQQLPGTAPKLLIYSNNQRPSGVPDRFSGSKSGTSASLAISGLQSEDEADYYCAAWDDSLNGLWVFGGGTKLTVL

SEQ ID NO: 60 - protein, 112 aa, synthetic construct
  REGION 1..112: note = Synthetic
DVVMTQSPLSLPVTPGEPASISCRSSQRLLHSNGYNYVDWYLQKPGQSPQLLIYLGSNRASGVPDRFSGSGSGTDFTLKISRVEAEDVGVYYCMQGTHWPWTFGQGTKVEIK

SEQ ID NO: 61 - protein, 107 aa, synthetic construct
  REGION 1..107: note = Synthetic
DIQMTQSPPSLSASVGDRVTITCQASQDINNYLNWYHQKPGKAPELLIYDASQLETGVPSRFSGSGSGTEFTFIISSLQPEDTGTYYCQQYDWLPLSYGGGTKVEIK

SEQ ID NO: 62 - protein, 111 aa, synthetic construct
  REGION 1..111: note = Synthetic
NFMLTQPHSVSGSPGKTITISCTRSSGNFASTYVQWYQQRPGSSPAIVIYDDDQRPSGVPDRFSGSIDRSSNSASLTISGLETEDEADYYCQSYDSSNFWVFGGGTKLTVL

SEQ ID NO: 63 - protein, 106 aa, synthetic construct
  REGION 1..106: note = Synthetic
EIVLTQSPSSLSASVGDRVTITCQASQDINNYLNWYQQKPGKAPKLLIYDASNLETGVPSKFSGSGSGTDFTFTISSLQPEDIATYYCQQYANLPSFGQGTKLEIK

SEQ ID NO: 64 - protein, 112 aa, synthetic construct
  REGION 1..112: note = Synthetic
EIVLTQSPLSLPVTPGEPASISCRSSQSLLHSNGYNYLDWYLQKPGQSPQLLIYLGSNRASGVPDRFSGSGSGTDFTLSISRVEAEDVGVYYCMQALQTPPTFGQGTKVEIK

SEQ ID NO: 65 - protein, 107 aa, synthetic construct
  REGION 1..107: note = Synthetic
DIQLTQSPSSLSASVGDRVTVTCQASQDIGHNLNWYQQRPGKAPQLLIYDASNLETGVPSRFSGSGSGTQFTFTISSLQPEDIATYYCQQYDFLPPDFGPGTKVEIK

SEQ ID NO: 66 - protein, 120 aa, synthetic construct
  REGION 1..120: note = Synthetic
QVQLQQWGAGLLKPSETLSLTCAVYGGSFSGYYWSWIRQPPGKGLEWIGEINHSGSTNYNPSLKSRATISVDTSKNQFSLKLSSVTAADTAVYYCATSPGGYSGGYFQHWGQGTLVTVSS

SEQ ID NO: 67 - protein, 120 aa, synthetic construct
  REGION 1..120: note = Synthetic
QVQLQESGPGLVKPSETLSLTCTVSGGSISSSSYYWGWIRQPPGKGLEWIGSIYYSGSTYYNPSLKSRVTISVDTSKNQFSLKLSSVTAADTAVYYCAGDILTGYALDYWGQGTLVTVSS

SEQ ID NO: 68 - protein, 120 aa, synthetic construct
  REGION 1..120: note = Synthetic
QVQLQQWGAGLLKPSETLSLTCAVYGGSFSGYYWSWIRQPPGKGLEWIGEINHFGSTNYNPSLKSRATISVDTSKNQFSLKLSSVTAADTAVYYCATSPGGYSGGYFQHWGQGTLVTVSS

SEQ ID NO: 69 - protein, 120 aa, synthetic construct
  REGION 1..120: note = Synthetic
QVQLQQWGAGMLKPSETLSLTCAVYGGSFSGYYWSWIRQPPGKGLEWIGEINHFGSTNYNPSLKSRATISVDTSKNQFSLKLSSVTAADTAVYYCATSPGGYSGGYFQHWGQGTLVTVSS

SEQ ID NO: 70 - protein, 120 aa, synthetic construct
  REGION 1..120: note = Synthetic
EVQLVESGGGLVQPGGSLRLSCAASGGSFSGYYWSWVRQAPGKGLEWVSEINHSGSTNYNPSLKSRFTISRDNSKNTLYLQMNSLRAEDTAVYYCARSPGGYSGGYFQHWGQGTLVTVSS

SEQ ID NO: 71 - protein, 120 aa, synthetic construct
  REGION 1..120: note = Synthetic
QVQLQQSGAEVKKPGSSVKVSCKASGGTFSSYAISWVRQAPGQGLEWMGGFDPEDGETIYAQKFQGRVTMTEDTSTDTAYMELSSLRSEDTAVYYCATDLRIPYYYDNPWGQGTLVTVSS

SEQ ID NO: 72 - protein, 118 aa, synthetic construct
  REGION 1..118: note = Synthetic
QVQLVQSGGGVVQPGRSLRLSCAASGFTFSSYAMHWVRQAPGKGLEWVAVISYDGSNKYYADSVKGRFTISRDNSKNTLYLQMNSLRAEDTAVYYCARSLYYSHFDYWGQGTLVTVSS

SEQ ID NO: 73 - protein, 123 aa, synthetic construct
  REGION 1..123: note = Synthetic
EVQLVETGPGLVKPSGTLSLTCAVSGGSISSSNWWSWVRQPPGKGLEWIGEIYHSGSTNYNPSLKSRVTISVDKSKNQFSLKLSSVTAADTAVYYCAREGPLSSSGPGAFDIWGQGTMVTVSS

SEQ ID NO: 74 - protein, 114 aa, synthetic construct
  REGION 1..114: note = Synthetic
QVQLQESGGGVVQPGRSLRLSCAASGFTFSNYGMHWVRQAPGKGLEWVAVISYDGTNKYYADSVKGRFTISRDNSKNTVYLQMNSLRAEDTAVYYCAKDGFDIWGQGTMVTVSS

SEQ ID NO: 75 - protein, 120 aa, synthetic construct
  REGION 1..120: note = Synthetic
EVQLVQSGGGVVQPGRSLRLSCAASGFTFSSYGMHWVRQAPGKGLEWVAVISYDGSNKYYADSVKGRFTISRDNSKNTLYLQMNSLRAEDTAVYYCARDVYGDYGAFDYWGQGTLVTVSS

SEQ ID NO: 76 - protein, 117 aa, synthetic construct
  REGION 1..117: note = Synthetic
QLQLQESGGGVVQPGRSLRLSCAASGFTFSSYAMHWVRQAPGKGLEWVAVISYDGGNKYYADSVKGRFTISRDNSKNTLYLQMNSLRAEDTAVYYCAKSVAPPMDVWGKGTTVTVSS

SEQ ID NO: 77 - protein, 121 aa, synthetic construct
  REGION 1..121: note = Synthetic
QVQLQQWGAGLLKPSETLSLTCAVYGESFSGYYWSWIRQPPGKGLEWIGEINHSGSTNYNPSLKSRVTISVDTSKNQFSLKLSSVTAADTAVYYCARGPAGSSSSGYFDYWGQGTLVTVSS

SEQ ID NO: 78 - protein, 116 aa, synthetic construct
  REGION 1..116: note = Synthetic
QVQLQESGPGLVKPSQTLSLTCTVSGGSISSGGYYWTWIRQHPGQGLEWIGFISWSGTTYYNPSLKNRVTISADTSKNHFSLNLTSVTAADTAVYYCARGSGRLVWGQGTLVTVSS

SEQ ID NO: 79 - protein, 120 aa, synthetic construct
  REGION 1..120: note = Synthetic
EVQLVQSGAEVKKPGASVKVSCKASGYTFTGYYMHWVRQAPGQGLEWMGIINPSGGSTSYAQKFQGRVTMTRDTSTSTVYMELSSLRSEDTAVYYCARADTAMGDAFDIWGQGTMVTVSS

SEQ ID NO: 80 - protein, 118 aa, synthetic construct
  REGION 1..118: note = Synthetic
EVQLVQSGGGVVQPGRSLRLSCAASGFTFSSYAMHWVRQAPGKGLEWVAVISYDGSNKYYADSVKGRFTISRDNSKNTLYLQMNSLRAEDTAVYYCARSIGIGAFDIWGQGTMVTVSS

SEQ ID NO: 81 - protein, 121 aa, synthetic construct
  REGION 1..121: note = Synthetic
QVQLQQWGAGLLKPSETLSLTCTIYGGSFSGFYWSWIRQPPGKGLEWIGEINHSGSTNYNPSLKSRVTISVDTSKNQFSLKLSSVTAADTAIYYCARGPAGSTSSGYFDHWGQGTLVTVSS

SEQ ID NO: 82 - protein, 117 aa, synthetic construct
  REGION 1..117: note = Synthetic
QVQLQQWGAGLLKPSETLSLTCAVYGGSLSGYYWSWIRQPPGKGLEWIGEINHSGSTNYNPSLKSRVTISVDTSKNQFSLKLSSVTAADTAVYYCARGSSSYYMDVWGKGTTVTVSS

SEQ ID NO: 83 - protein, 116 aa, synthetic construct
  REGION 1..116: note = Synthetic
QVQLQQWGAGLLKPSETLSLTCAVYGGSFSGYYWSWIRQPPGKGLEWIGEINHSGSTNYNPSLKSRVTISVDTSKNQFSLKLSSVTAADTAVYYCARGGSAYFQHWGQGTLVTVSS

SEQ ID NO: 84 - protein, 121 aa, synthetic construct
  REGION 1..121: note = Synthetic
QVQLQQWGAGLLKPSETLSLTCAVYGGSFSGYYWSWIRQPPGKGLEWIGEINHSGSTNYNPSLKSRVTISEDASKKQFSLTLTSVTAADTAVYYCARGPAGTGSSGYFDYWGQGTLVTVSS

SEQ ID NO: 85 - protein, 112 aa, synthetic construct
  REGION 1..112: note = Synthetic
DVVMTQSPLSLPVTPGEPASISCRSSQSLLHSNGYNYLDWYLQKPGQSPQLLIYLGSNRDSGVPDRFSGSGSGTDFTLKISRVEAEDVGVYYCMQGTHWPWTFGQGTKVEIK

SEQ ID NO: 86 - protein, 108 aa, synthetic construct
  REGION 1..108: note = Synthetic
SYELTQPPSMSVSPGQTARITCSGDALPKQFAFWYQQKPGQAPVLVIYKDTERPSGIPERFSGSSSGTTVTLTITGVQAEDEADYYCQSPDSSGTVEVFGGGTKLTVL

SEQ ID NO: 87 - protein, 112 aa, synthetic construct
  REGION 1..112: note = Synthetic
DVVMTQSPLSLPVTPGESVSISCRSSQSLLHSNGYNYLDWYLQKPGQSPQLLIYLGSNRDSGVPDRFSGSGSGTDFTLKISRVEAEDVGVYYCMQGTHWPWTFGQGTKVEIK

SEQ ID NO: 88 - protein, 112 aa, synthetic construct
  REGION 1..112: note = Synthetic
DIQMTQSPSSLSASVGDRVTITCRSSQSLLHSNGYNYLDWYQQKPGKAPKLLIYLGSNRDSGVPSRFSGSGSGTDFTLTISSLQPEDFATYYCMQGTHWPWTFGQGTKVEIK

SEQ ID NO: 89 - protein, 112 aa, synthetic construct
  REGION 1..112: note = Synthetic
EIVLTQSPLSLPVTPGEPASISCRSSQSLLHSNGYNYLNWYLQKPGQSPQLLIYLGSDRASGVPDRFSGSGSGTDFTLKISRVEAEDVGVYYCMQALQTPTTFGGGTKVEIK

SEQ ID NO: 90 - protein, 112 aa, synthetic construct
  REGION 1..112: note = Synthetic
EIVMTQSPLSLPVTPGEPASISCRSSQSLLHSNGYNYLDWYLQKPGQSPQLLMYLVSNRASGVPERFSGSGSGTDFTLKISRVEAEDVGVYYCMQTLQTPLSFGQGTKLEIK

SEQ ID NO: 91 - protein, 107 aa, synthetic construct
  REGION 1..107: note = Synthetic
EIVLTQSPATLSLSPGERATLSCRASQSVSYHLAWYQQKPGQAPRLLIYDTSNRASGIPARFSGSGSGTDFTLTINSLEPEDFAVYYCQQRYDWPLTFGGGTKVEIK

SEQ ID NO: 92 - protein, 107 aa, synthetic construct
  REGION 1..107: note = Synthetic
DIQMTQSPSFLSASVGDRVTITCRASQSISSWLAWYQQKPGKAPKLLIYDASRLEDGVPSRFSGTGFGTDFTFTITTLQPDDIATYYCQQYDDLPYTFGQGTTVDIK

SEQ ID NO: 93 - protein, 112 aa, synthetic construct
  REGION 1..112: note = Synthetic
DVVMTQSPVSLPVTLGQPASISCKSSQSLLYFNGNTYLSWFQQRPGQSPRRLFYQVSNRDSGVPDRFSGSGSDTDFTLTISRVEAEDVGVYFCMQGTQWPPTFGQGTKVEIK

SEQ ID NO: 94 - protein, 112 aa, synthetic construct
  REGION 1..112: note = Synthetic
DVVMTQSPLSLPVTPGEPASISCRSSQSLLHSNGYNYLDWYLQKPGQSPHLLIYLGSNRASGVPDRFSGSGSGTDFTLKISRVEAEDVGVYFCMQALRTPYTFGQGTKLEIK

SEQ ID NO: 95 - protein, 112 aa, synthetic construct
  REGION 1..112: note = Synthetic
DVVMTQSPLSLPVTLGQPASISCRSSQSLVHSNGYNYLDWYLQKPGQSPQLLIYLGSNRASGVPDRFSGSGSGTDFTLKISRVEAEDVGVYYCLQGSHWPWTFGQGTKVEIK

SEQ ID NO: 96 - protein, 106 aa, synthetic construct
  REGION 1..106: note = Synthetic
ETTLTQSPATLSVSPGERATLSCRASQSVSSNLAWYQQKPGQAPRLLIYGASSGATGIPDRFSGSGSGTDFTLTISRLEPEDFAVYYCQLYGSSLAFGGGTKVEIK

SEQ ID NO: 97 - protein, 113 aa, synthetic construct
  REGION 1..113: note = Synthetic
DIVMTHTPLSLPVTPGEPASISCRSSQTLFDSDDGKTYLDWYLQKPGQSPQLLMYTTSSRASGVPDRFSGSGSGTDFTLKISRVEAEDVGVYYCMQRLQFPLTFGQGTRLEFK

SEQ ID NO: 98 - protein, 112 aa, synthetic construct
  REGION 1..112: note = Synthetic
DVVMTQSPLSLPVTPGEPASISCRATQSLLHGNGHNYLDWYLQKPGQSPQLLIYLGSNRASGVPDRFSGSGSGTDFTLKISRVEAEDVGVYYCMQTLETPVTFGPGTKVDIK

SEQ ID NO: 99 - protein, 112 aa, synthetic construct
  REGION 1..112: note = Synthetic
DVVMTQSPLSLPVTPGEPASISCRSSQSLLHSNGYNYLDWYLQKPGQSPQLLIYLGSNRASGVPDRFSGSGSGTDFTLKISRVEAEDVGVYYCMQGTHWPWTFGQGTKVEIK

SEQ ID NO: 100 - protein, 112 aa, synthetic construct
  REGION 1..112: note = Synthetic
DVVMTQSPLSLPVTPGEPASISCRSSQSLLHSNGYNYLDWYLQKPGQSPQLLIYLGSNRASGVPDRFSGSGSGTDFTLKISRVEAEDVGVYYCLQGTHWPWTFGQGTKVEIK

SEQ ID NO: 101 - protein, 112 aa, synthetic construct
  REGION 1..112: note = Synthetic
DVVMTQSPLSLPVTLGQPASISCRSSQSLLHSNGNNYLDWYLQKPGQSPQLLIYLGSNRASGVPDRFSGSGSGTDFTLKISRVEAEDVGIYYCMQGTHWPWTFGQGTKVEIE

SEQ ID NO: 102 - protein, 112 aa, synthetic construct
  REGION 1..112: note = Synthetic
EIVLTQSPLSLPVTPGEPASISCRSSQSLLHSNGYNYLDWYLQKPGQSPQLLIYLASNRASGVPDRFSGSGSGTDFTLKISRVEAEDVGVYYCMQGTHWPWTFGQGTKVEVK

SEQ ID NO: 103 - protein, 5 aa, synthetic construct
  REGION 1..5: note = Synthetic
NYYMH

SEQ ID NO: 104 - protein, 5 aa, synthetic construct
  REGION 1..5: note = Synthetic
TYAMH

SEQ ID NO: 105 - protein, 5 aa, synthetic construct
  REGION 1..5: note = Synthetic
SYAMS

SEQ ID NO: 106 - protein, 5 aa, synthetic construct
  REGION 1..5: note = Synthetic
GYYWS

SEQ ID NO: 107 - protein, 5 aa, synthetic construct
  REGION 1..5: note = Synthetic
NYYIH

SEQ ID NO: 108 - protein, 5 aa, synthetic construct
  REGION 1..5: note = Synthetic
SYAMH

SEQ ID NO: 109 - protein, 7 aa, synthetic construct
  REGION 1..7: note = Synthetic
SRSDHWG

SEQ ID NO: 110 - protein, 5 aa, synthetic construct
  REGION 1..5: note = Synthetic
SYYWS

SEQ ID NO: 111 - protein, 5 aa, synthetic construct
  REGION 1..5: note = Synthetic
SYDLH

SEQ ID NO: 112 - protein, 6 aa, synthetic construct
  REGION 1..6: note = Synthetic
SSNWWS

SEQ ID NO: 113 - protein, 7 aa, synthetic construct
  REGION 1..7: note = Synthetic
GNSATWN

SEQ ID NO: 114 - protein, 6 aa, synthetic construct
  REGION 1..6: note = Synthetic
SGSWWS

SEQ ID NO: 115 - protein, 5 aa, synthetic construct
  REGION 1..5: note = Synthetic
SYGIS

SEQ ID NO: 116 - protein, 17 aa, synthetic construct
  REGION 1..17: note = Synthetic
AIMPSRGGTSYPQKFQG

SEQ ID NO: 117 - protein, 17 aa, synthetic construct
  REGION 1..17: note = Synthetic
WINTGNGNTKYSQNFQG

SEQ ID NO: 118 - protein, 17 aa, synthetic construct
  REGION 1..17: note = Synthetic
AISGSGGSTYYADSVKG

SEQ ID NO: 119 - protein, 16 aa, synthetic construct
  REGION 1..16: note = Synthetic
EINHSGSTNYNPSLKS

SEQ ID NO: 120 - protein, 17 aa, synthetic construct
  REGION 1..17: note = Synthetic
VISYDGSNKYYADSVKG

SEQ ID NO: 121 - protein, 16 aa, synthetic construct
  REGION 1..16: note = Synthetic
SISYSGSTYYNPSLKS

SEQ ID NO: 122 - protein, 17 aa, synthetic construct
  REGION 1..17: note = Synthetic
AISGSGGSTHYADSVKG

SEQ ID NO: 123 - protein, 17 aa, synthetic construct
  REGION 1..17: note = Synthetic
LISYDGSNKYYADSVKG

SEQ ID NO: 124 - protein, 16 aa, synthetic construct
  REGION 1..16: note = Synthetic
EIYHSGSTNYNPSLKS

SEQ ID NO: 125 - protein, 18 aa, synthetic construct
  REGION 1..18: note = Synthetic
RTYYRSKWNHDYAESVKS

SEQ ID NO: 126 - protein, 16 aa, synthetic construct
  REGION 1..16: note = Synthetic
EIYHNGNTNYNPSLKS

SEQ ID NO: 127 - protein, 17 aa, synthetic construct
  REGION 1..17: note = Synthetic
WISAYNGNTNYAQKLQG

SEQ ID NO: 128 - protein, 12 aa, synthetic construct
  REGION 1..12: note = Synthetic
GEYYYDSSGYYY

SEQ ID NO: 129 - protein, 11 aa, synthetic construct
  REGION 1..11: note = Synthetic
DLGQLERLYFW

SEQ ID NO: 130 - protein, 5 aa, synthetic construct
  REGION 1..5: note = Synthetic
DLGDY

SEQ ID NO: 131 - protein, 9 aa, synthetic construct
  REGION 1..9: note = Synthetic
SSSGDAFDI

SEQ ID NO: 132 - protein, 12 aa, synthetic construct
  REGION 1..12: note = Synthetic
GEYYYDSSGYYN

SEQ ID NO: 133 - protein, 17 aa, synthetic construct
  REGION 1..17: note = Synthetic
DLGYSSSWYYYYYGMDV

SEQ ID NO: 134 - protein, 10 aa, synthetic construct
  REGION 1..10: note = Synthetic
ESHPAAALVG

SEQ ID NO: 135 - skipped

SEQ ID NO: 136 - protein, 9 aa, synthetic construct
  REGION 1..9: note = Synthetic
GSNLDWFDP

SEQ ID NO: 137 - protein, 10 aa, synthetic construct
  REGION 1..10: note = Synthetic
ADRRFGELRY

SEQ ID NO: 138 - protein, 12 aa, synthetic construct
  REGION 1..12: note = Synthetic
EPITGTSDLFDY

SEQ ID NO: 139 - protein, 8 aa, synthetic construct
  REGION 1..8: note = Synthetic
EGGLREEH

SEQ ID NO: 140 - protein, 9 aa, synthetic construct
  REGION 1..9: note = Synthetic
GTDTAMADY

SEQ ID NO: 141 - protein, 8 aa, synthetic construct
  REGION 1..8: note = Synthetic
DSKSAFDI

SEQ ID NO: 142 - protein, 6 aa, synthetic construct
  REGION 1..6: note = Synthetic
VSGFDY

SEQ ID NO: 143 - protein, 17 aa, synthetic construct
  REGION 1..17: note = Synthetic
APDYGDSSNYYYYYMDV

SEQ ID NO: 144 - protein, 18 aa, synthetic construct
  REGION 1..18: note = Synthetic
DTSGDYSSGWYLGVPFDY

SEQ ID NO: 145 - protein, 14 aa, synthetic construct
  REGION 1..14: note = Synthetic
TGTSSDIGHYDFVS

SEQ ID NO: 146 - protein, 11 aa, synthetic construct
  REGION 1..11: note = Synthetic
RAGQAISSWLA

SEQ ID NO: 147 - protein, 11 aa, synthetic construct
  REGION 1..11: note = Synthetic
QASQDIGNYLN

SEQ ID NO: 148 - protein, 13 aa, synthetic construct
  REGION 1..13: note = Synthetic
TRSSGSIASNYVQ

SEQ ID NO: 149 - protein, 14 aa, synthetic construct
  REGION 1..14: note = Synthetic
TGTSSDIGDYDFVS

SEQ ID NO: 150 - protein, 14 aa, synthetic construct
  REGION 1..14: note = Synthetic
TGTSSDIGHYDFIS

SEQ ID NO: 151 - protein, 16 aa, synthetic construct
  REGION 1..16: note = Synthetic
RSSQSLLHSNGYNYLD

SEQ ID NO: 152 - protein, 11 aa, synthetic construct
  REGION 1..11: note = Synthetic
QASQDISDYLN

SEQ ID NO: 153 - protein, 11 aa, synthetic construct
  REGION 1..11: note = Synthetic
RASQSISSYLN

SEQ ID NO: 154 - protein, 14 aa, synthetic construct
  REGION 1..14: note = Synthetic
TGGSSNVGAGYDVH

SEQ ID NO: 155 - protein, 13 aa, synthetic construct
  REGION 1..13: note = Synthetic
TGSSGSIASSYVQ

SEQ ID NO: 156 - protein, 14 aa, synthetic construct
  REGION 1..14: note = Synthetic
TGSRSNIGAGYDVH

SEQ ID NO: 157 - protein, 13 aa, synthetic construct
  REGION 1..13: note = Synthetic
SGSSSNIGSNTVN

SEQ ID NO: 158 - protein, 16 aa, synthetic construct
  REGION 1..16: note = Synthetic
RSSQRLLHSNGYNYVD

SEQ ID NO: 159 - protein, 11 aa, synthetic construct
  REGION 1..11: note = Synthetic
QASQDINNYLN

SEQ ID NO: 160 - protein, 13 aa, synthetic construct
  REGION 1..13: note = Synthetic
TRSSGNFASTYVQ

SEQ ID NO: 161 - protein, 11 aa, synthetic construct
  REGION 1..11: note = Synthetic
QASQDIGHNLN

SEQ ID NO: 162 - protein, 7 aa, synthetic construct
  REGION 1..7: note = Synthetic
DINNRPS

SEQ ID NO: 163 - protein, 7 aa, synthetic construct
  REGION 1..7: note = Synthetic
KASNLES

SEQ ID NO: 164 - protein, 7 aa, synthetic construct
  REGION 1..7: note = Synthetic
DASNLET

SEQ ID NO: 165 - protein, 7 aa, synthetic construct
  REGION 1..7: note = Synthetic
DDNQRPT

SEQ ID NO: 166 - protein, 7 aa, synthetic construct
  REGION 1..7: note = Synthetic
DNNNRPS

SEQ ID NO: 167 - protein, 7 aa, synthetic construct
  REGION 1..7: note = Synthetic
DFNNRPS

SEQ ID NO: 168 - protein, 7 aa, synthetic construct
  REGION 1..7: note = Synthetic
DINNRAS

SEQ ID NO: 169 - protein, 7 aa, synthetic construct
  REGION 1..7: note = Synthetic
LGSSRAS

SEQ ID NO: 170 - protein, 7 aa, synthetic construct
  REGION 1..7: note = Synthetic
DNTNRPS

SEQ ID NO: 171 - protein, 7 aa, synthetic construct
  REGION 1..7: note = Synthetic
ADNQRPS

SEQ ID NO: 172 - protein, 7 aa, synthetic construct
  REGION 1..7: note = Synthetic
DNSNRPS

SEQ ID NO: 173 - protein, 7 aa, synthetic construct
  REGION 1..7: note = Synthetic
SNNQRPS

SEQ ID NO: 174 - protein, 7 aa, synthetic construct
  REGION 1..7: note = Synthetic
LGSNRAS

SEQ ID NO: 175 - protein, 7 aa, synthetic construct
  REGION 1..7: note = Synthetic
DASQLET

SEQ ID NO: 176 - protein, 7 aa, synthetic construct
  REGION 1..7: note = Synthetic
DDDQRPS

SEQ ID NO: 177 - protein, 10 aa, synthetic construct
  REGION 1..10: note = Synthetic
SAYTSSDTLV

SEQ ID NO: 178 - protein, 9 aa, synthetic construct
  REGION 1..9: note = Synthetic
QQYQSYPYT

SEQ ID NO: 179 - protein, 9 aa, synthetic construct
  REGION 1..9: note = Synthetic
LQLYDYPLT

SEQ ID NO: 180 - protein, 9 aa, synthetic construct
  REGION 1..9: note = Synthetic
QSSHSTAVV

SEQ ID NO: 181 - protein, 10 aa, synthetic construct
  REGION 1..10: note = Synthetic
SAYTSSDTVV

SEQ ID NO: 182 - protein, 9 aa, synthetic construct
  REGION 1..9: note = Synthetic
MQALQTPRT

SEQ ID NO: 183 - protein, 8 aa, synthetic construct
  REGION 1..8: note = Synthetic
QQYEDLPS

SEQ ID NO: 184 - protein, 9 aa, synthetic construct
  REGION 1..9: note = Synthetic
LQDYIYPWT

SEQ ID NO: 185 - protein, 11 aa, synthetic construct
  REGION 1..11: note = Synthetic
QSYDSSLRASV

SEQ ID NO: 186 - protein, 10 aa, synthetic construct
  REGION 1..10: note = Synthetic
QSFDSSLYMI

SEQ ID NO: 187 - protein, 11 aa, synthetic construct
  REGION 1..11: note = Synthetic
QSFDSSLRGVV

SEQ ID NO: 188 - protein, 12 aa, synthetic construct
  REGION 1..12: note = Synthetic
AAWDDSLNGLWV

SEQ ID NO: 189 - protein, 9 aa, synthetic construct
  REGION 1..9: note = Synthetic
MQGTHWPWT

SEQ ID NO: 190 - protein, 9 aa, synthetic construct
  REGION 1..9: note = Synthetic
QQYDWLPLS

SEQ ID NO: 191 - protein, 10 aa, synthetic construct
  REGION 1..10: note = Synthetic
QSYDSSNFWV

SEQ ID NO: 192 - protein, 8 aa, synthetic construct
  REGION 1..8: note = Synthetic
QQYANLPS

SEQ ID NO: 193 - protein, 9 aa, synthetic construct
  REGION 1..9: note = Synthetic
MQALQTPPT

SEQ ID NO: 194 - protein, 9 aa, synthetic construct
  REGION 1..9: note = Synthetic
QQYDFLPPD

SEQ ID NO: 195 - protein, 7 aa, synthetic construct
  REGION 1..7: note = Synthetic
SSSYYWG

SEQ ID NO: 196 - protein, 5 aa, synthetic construct
  REGION 1..5: note = Synthetic
SYAIS

SEQ ID NO: 197 - protein, 5 aa, synthetic construct
  REGION 1..5: note = Synthetic
NYGMH

SEQ ID NO: 198 - protein, 5 aa, synthetic construct
  REGION 1..5: note = Synthetic
SYGMH

SEQ ID NO: 199 - protein, 7 aa, synthetic construct
  REGION 1..7: note = Synthetic
SGGYYWT

SEQ ID NO: 200 - protein, 5 aa, synthetic construct
  REGION 1..5: note = Synthetic
GYYMH

SEQ ID NO: 201 - protein, 5 aa, synthetic construct
  REGION 1..5: note = Synthetic
GFYWS

SEQ ID NO: 202 - protein, 15 aa, synthetic construct
  REGION 1..15: note = Synthetic
SIYYSGSTYYNPSLK

SEQ ID NO: 203 - protein, 16 aa, synthetic construct
  REGION 1..16: note = Synthetic
EINHFGSTNYNPSLKS

SEQ ID NO: 204 - protein, 17 aa, synthetic construct
  REGION 1..17: note = Synthetic
GFDPEDGETIYAQKFQG

SEQ ID NO: 205 - skipped

SEQ ID NO: 206 - protein, 17 aa, synthetic construct
  REGION 1..17: note = Synthetic
VISYDGTNKYYADSVKG

SEQ ID NO: 207 - protein, 17 aa, synthetic construct
  REGION 1..17: note = Synthetic
VISYDGGNKYYADSVKG

SEQ ID NO: 208 - protein, 16 aa, synthetic construct
  REGION 1..16: note = Synthetic
FISWSGTTYYNPSLKN

SEQ ID NO: 209 - protein, 17 aa, synthetic construct
  REGION 1..17: note = Synthetic
IINPSGGSTSYAQKFQG

SEQ ID NO: 210 - protein, 12 aa, synthetic construct
  REGION 1..12: note = Synthetic
SPGGYSGGYFQH

SEQ ID NO: 211 - protein, 10 aa, synthetic construct
  REGION 1..10: note = Synthetic
DILTGYALDY

SEQ ID NO: 212 - protein, 11 aa, synthetic construct
  REGION 1..11: note = Synthetic
DLRIPYYYDNP

SEQ ID NO: 213 - protein, 9 aa, synthetic construct
  REGION 1..9: note = Synthetic
SLYYSHFDY

SEQ ID NO: 214 - protein, 14 aa, synthetic construct
  REGION 1..14: note = Synthetic
EGPLSSSGPGAFDI

SEQ ID NO: 215 - protein, 5 aa, synthetic construct
  REGION 1..5: note = Synthetic
DGFDI

SEQ ID NO: 216 - protein, 11 aa, synthetic construct
  REGION 1..11: note = Synthetic
DVYGDYGAFDY

SEQ ID NO: 217 - protein, 8 aa, synthetic construct
  REGION 1..8: note = Synthetic
SVAPPMDV

SEQ ID NO: 218 - protein, 13 aa, synthetic construct
  REGION 1..13: note = Synthetic
GPAGSSSSGYFDY

SEQ ID NO: 219 - protein, 6 aa, synthetic construct
  REGION 1..6: note = Synthetic
GSGRLV

SEQ ID NO: 220 - protein, 11 aa, synthetic construct
  REGION 1..11: note = Synthetic
ADTAMGDAFDI

SEQ ID NO: 221 - protein, 9 aa, synthetic construct
  REGION 1..9: note = Synthetic
SIGIGAFDI

SEQ ID NO: 222 - protein, 13 aa, synthetic construct
  REGION 1..13: note = Synthetic
GPAGSTSSGYFDH

SEQ ID NO: 223 - protein, 9 aa, synthetic construct
  REGION 1..9: note = Synthetic
GSSSYYMDV

SEQ ID NO: 224 - protein, 8 aa, synthetic construct
  REGION 1..8: note = Synthetic
GGSAYFQH

SEQ ID NO: 225 - protein, 13 aa, synthetic construct
  REGION 1..13: note = Synthetic
GPAGTGSSGYFDY

SEQ ID NO: 226 - protein, 11 aa, synthetic construct
  REGION 1..11: note = Synthetic
SGDALPKQFAF

SEQ ID NO: 227 - protein, 16 aa, synthetic construct
  REGION 1..16: note = Synthetic
RSSQSLLHSNGYNYLN

SEQ ID NO: 228 - protein, 11 aa, synthetic construct
  REGION 1..11: note = Synthetic
RASQSVSYHLA

SEQ ID NO: 229 - protein, 11 aa, synthetic construct
  REGION 1..11: note = Synthetic
RASQSISSWLA

SEQ ID NO: 230 - protein, 16 aa, synthetic construct
  REGION 1..16: note = Synthetic
KSSQSLLYFNGNTYLS

SEQ ID NO: 231 - protein, 16 aa, synthetic construct
  REGION 1..16: note = Synthetic
RSSQSLVHSNGYNYLD

SEQ ID NO: 232 - protein, 11 aa, synthetic construct
  REGION 1..11: note = Synthetic
RASQSVSSNLA

SEQ ID NO: 233 - protein, 17 aa, synthetic construct
  REGION 1..17: note = Synthetic
RSSQTLFDSDDGKTYLD

SEQ ID NO: 234 - protein, 16 aa, synthetic construct
  REGION 1..16: note = Synthetic
RATQSLLHGNGHNYLD

SEQ ID NO: 235 - protein, 16 aa, synthetic construct
  REGION 1..16: note = Synthetic
RSSQSLLHSNGNNYLD

SEQ ID NO: 236 - protein, 7 aa, synthetic construct
  REGION 1..7: note = Synthetic
LGSNRDS

SEQ ID NO: 237 - protein, 7 aa, synthetic construct
  REGION 1..7: note = Synthetic
KDTERPS

SEQ ID NO: 238 - protein, 7 aa, synthetic construct
  REGION 1..7: note = Synthetic
LGSDRAS

SEQ ID NO: 239 - protein, 7 aa, synthetic construct
  REGION 1..7: note = Synthetic
LVSNRAS

SEQ ID NO: 240 - protein, 7 aa, synthetic construct
  REGION 1..7: note = Synthetic
DTSNRAS

SEQ ID NO: 241 - protein, 7 aa, synthetic construct
  REGION 1..7: note = Synthetic
DASRLED

SEQ ID NO: 242 - protein, 7 aa, synthetic construct
  REGION 1..7: note = Synthetic
QVSNRDS

SEQ ID NO: 243 - protein, 7 aa, synthetic construct
  REGION 1..7: note = Synthetic
GASSGAT

SEQ ID NO: 244 - protein, 7 aa, synthetic construct
  REGION 1..7: note = Synthetic
TTSSRAS

SEQ ID NO: 245 - protein, 7 aa, synthetic construct
  REGION 1..7: note = Synthetic
LASNRAS

SEQ ID NO: 246 - skipped

SEQ ID NO: 247 - protein, 11 aa, synthetic construct
  REGION 1..11: note = Synthetic
QSPDSSGTVEV

SEQ ID NO: 248 - protein, 9 aa, synthetic construct
  REGION 1..9: note = Synthetic
MQALQTPTT

SEQ ID NO: 249 - protein, 9 aa, synthetic construct
  REGION 1..9: note = Synthetic
MQTLQTPLS

SEQ ID NO: 250 - protein, 9 aa, synthetic construct
  REGION 1..9: note = Synthetic
QQRYDWPLT

SEQ ID NO: 251 - protein, 9 aa, synthetic construct
  REGION 1..9: note = Synthetic
QQYDDLPYT

SEQ ID NO: 252 - protein, 9 aa, synthetic construct
  REGION 1..9: note = Synthetic
MQGTQWPPT

SEQ ID NO: 253 - protein, 9 aa, synthetic construct
  REGION 1..9: note = Synthetic
MQALRTPYT

SEQ ID NO: 254 - protein, 9 aa, synthetic construct
  REGION 1..9: note = Synthetic
LQGSHWPWT

SEQ ID NO: 255 - protein, 8 aa, synthetic construct
  REGION 1..8: note = Synthetic
QLYGSSLA

SEQ ID NO: 256 - protein, 9 aa, synthetic construct
  REGION 1..9: note = Synthetic
MQRLQFPLT

SEQ ID NO: 257 - protein, 9 aa, synthetic construct
  REGION 1..9: note = Synthetic
MQTLETPVT

SEQ ID NO: 258 - protein, 9 aa, synthetic construct
  REGION 1..9: note = Synthetic
LQGTHWPWT

SEQ ID NO: 259 - protein, 113 aa, synthetic construct
  REGION 1..113: note = Synthetic
PCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAK

SEQ ID NO: 260 - protein, 107 aa, synthetic construct
  REGION 1..107: note = Synthetic
GQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 261 - protein, 113 aa, synthetic construct; identical to SEQ ID NO: 259
  REGION 1..113: note = Synthetic

SEQ ID NO: 262 - protein, 107 aa, synthetic construct
  REGION 1..107: note = Synthetic
GQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 263 - protein, 113 aa, synthetic construct; identical to SEQ ID NO: 259
  REGION 1..113: note = Synthetic

SEQ ID NO: 264 - protein, 107 aa, synthetic construct; identical to SEQ ID NO: 262
  REGION 1..107: note = Synthetic

SEQ ID NO: 265 - protein, 480 aa, synthetic construct
  REGION 1..480: note = Synthetic
EVQLVQSGAEVKKPGSSVKVSCKASGYTFTNYYMHWVRQAPGQGLEWMGAIMPSRGGTSYPQKFQGRVTMTGDTSTSTVYMELSSLRSEDTAVYYCARGEYYYDSSGYYYWGQGTLVTVSSGGGGSGGGGSGGGGSQSALTQPASVSGSPGQSIAISCTGTSSDIGHYDFVSWYQQHPGTAPKLIIYDINNRPSGISNRFSGSKSDNMASLTISGLQPEDEADYYCSAYTSSDTLVFGGGTKLTVLNSGAGTAAATHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 266 - protein, 241 aa, synthetic construct
  REGION 1..241: note = Synthetic
EVQLVQSGTEVKKPGASVKVSCKASGYTFTTYAMHWVRQAPGQSLEWMGWINTGNGNTKYSQNFQGRVTMTRDTSISTAYMELSRLRSDDTAVYYCARDLGQLERLYFWGQGTLVTVSSGGGGSGGGGSGGGGSDIQMTQSPSTLSASVGDRVTLSCRAGQAISSWLAWYQQKPGKAPKLLIYKASNLESGVPSRFSGGGSGAEFTLTISSLQPDDFATYYCQQYQSYPYTFGQGTKLEIR

SEQ ID NO: 267 - protein, 236 aa, synthetic construct
  REGION 1..236: note = Synthetic
HVQLVETGGGLVQPGGSLRLSCAASGFTFSSYAMSWVRQAPGKGLEWVSAISGSGGSTYYADSVKGRFTISRDNSKNTLYLQMNSLRAEDTAVYYCARDLGDYWGQGTLVTVSSGGGGSGGGGSGGGGSDIQLTQSPSSLSASVGDRVTITCQASQDIGNYLNWYQLKPGKAPKLLIYDASNLETGVPSRFSGSGSGTDFTFTISSLQPEDIATYYCLQLYDYPLTFGGGTKVEIK

SEQ ID NO: 268 - protein, 242 aa, synthetic construct
  REGION 1..242: note = Synthetic
QVQLQQWGAGLLKPSETLSLTCAVYGGSFSGYYWSWIRQPPGKGLEWIGEINHSGSTNYNPSLKSRVTISVDTSKNQFSLKLSSVTAADTAVYYCARSSSGDAFDIWGQGTMVTVSSGGGGSGGGGSGGGGSNFMLTQPHSVSESPGKTVTISCTRSSGSIASNYVQWYQQRPGSSPTTVIFDDNQRPTGVPDRFSAAIDTSSSSASLTISGLTAEDEADYYCQSSHSTAVVFGGGTKLTVL

SEQ ID NO: 269 - protein, 246 aa, synthetic construct
  REGION 1..246: note = Synthetic
EVQLVQSGAEVKKPGSSVKVSCKASGYTFTNYYMHWVRQAPGQGLEWMGAIMPSRGGTSYPQKFQGRVTMTGDTSTSTVYMELSSLRSEDTAVYYCARGEYYYDSSGYYYWGQGTLVTVSSGGGGSGGGGSGGGGSQSALTQPASVSGSPGQSIAISCTGTSSDIGDYDFVSWYQQHPGTAPKLIIYDINNRPSGISNRFSGSKSDNMASLTISGLQPEDEADYYCSAYTSSDTLVFGGGTKLTVL

SEQ ID NO: 270 - protein, 246 aa, synthetic construct
  REGION 1..246: note = Synthetic
EVQLVQSGAEVKKPGSSVKVSCKASGYTFTNYYMHWVRQAPGQGLEWMGAIMPSRGGTSYPQKFQGRVTMTGDTSTSTVYMELSSLRSEDTAVYYCARGEYYYDSSGYYNWGQGTLVTVSSGGGGSGGGGSGGGGSQSALTQPASVSGSPGQSIAISCTGTSSDIGHYDFVSWYQQHPGTAPKLIIYDINNRPSGISNRFSGSKSDNMASLTISGLQPEDEADYYCSAYTSSDTVVFGGGTKLTVL

SEQ ID NO: 271 - protein, 246 aa, synthetic construct
  REGION 1..246: note = Synthetic
EVQLVQSGAEVKKPGSSVKVSCKASGYTFTNYYMHWVRQAPGQGLEWMGAIMPSRGGTSYPQKFQGRVTMTGDTSTSTVYMELSSLRSEDTAVYYCARGEYYYDSSGYYYWGQGTLVTVSSGGGGSGGGGSGGGGSQSALTQPASVSGSPGQSIAISCTGTSSDIGHYDFVSWYQQHPGTAPKLIIYDNNNRPSGISNRFSGSKSDNMASLTISGLQPEDEADYYCSAYTSSDTLVFGGGTKLTVL

SEQ ID NO: 272 - protein, 246 aa, synthetic construct
  REGION 1..246: note = Synthetic
EVQLVQSGAEVKKPGSSVKVSCKASGYTFTNYYMHWVRQAPGQGLEWMGAIMPSRGGTSYPQKFQGRVTMTGDTSTSTVYMELSSLRSEDTAVYYCARGEYYYDSSGYYYWGQGTLVTVSSGGGGSGGGGSGGGGSQSALTQPASVSGSPGQSITISCTGTSSDIGHYDFVSWYQQHPGTAPKLIIYDINNRPSGISNRFSGSKSDNMASLTISGLQPEDEADYYCSAYTSSDTVVFGGGTKLTVL

SEQ ID NO: 273 - protein, 246 aa, synthetic construct
  REGION 1..246: note = Synthetic
EVQLVQSGAEVKKPGSSVKVSCKASGYTFTNYYIHWVRQAPGQGLEWMGAIMPSRGGTSYPQKFQGRVTMTGDTSTSTVYMELSSLRSEDTAVYYCARGEYYYDSSGYYYWGQGTLVTVSSGGGGSGGGGSGGGGSQSALTQPASVSGSPGQSIAISCTGTSSDIGHYDFVSWYQQHPGTAPKLIIYDINNRPSGISNRFSGSKSDNMASLTISGLQPEDEADYYCSAYTSSDTVVFGGGTKLTVL

SEQ ID NO: 274 - protein, 246 aa, synthetic construct
  REGION 1..246: note = Synthetic
EVQLVQSGAEVKKPGSSVKVSCKASGYTFTNYYMHWVRQPPGQGLEWMGAIMPSRGGTSYPQKFQGRVTMTGDTSTSTVYMELSSLRSEDTAVYYCARGEYYYDSSGYYYWGQGTLVTVSSGGGGSGGGGSGGGGSQSALTQPASVSGSPGQSIAISCTGTSSDIGHYDFVSWYQQHPGTAPKLIIYDINNRPSGISNRFSGSKSDNMASLTISGLQPEDEADYYCSAYTSSDTVVFGGGTKLTVL

SEQ ID NO: 275 - protein, 246 aa, synthetic construct
  REGION 1..246: note = Synthetic
EVQLVQSGAEVKKPGSTVKVSCKASGYTFTNYYMHWVRQAPGQGLEWMGAIMPSRGGTSYPQKFQGRVTMTGDTSTSTVYMELSSLRSEDTAVYYCARGEYYYDSSGYYYWGQGTLVTVSSGGGGSGGGGSGGGGSQSALTQPASVSGSPGQSIAISCTGTSSDIGHYDFISWYQQHPGTAPKLIIYDFNNRPSGISNRFSGSKSDNMASLTISGLQPEDEADYYCSAYTSSDTLVFGGGTKLTVL

SEQ ID NO: 276 - protein, 246 aa, synthetic construct
  REGION 1..246: note = Synthetic
EVQLVQSGAEVKKPGSSVKVSCKASGYTFTNYYMHWVRQAPGQGLEWMGAIMPSRGGTSYPQKFQGRVTMTGDTSTSTVYMELSSLRSEDTAVYYCARGEYYYDSSGYYYWGQGTPVTVSSGGGGSGGGGSGGGGSQSALTQPASVSGSPGQSIAISCTGTSSDIGDYDFVSWYQQHPGTAPKLIIYDINNRPSGISNRFSGSKSDNMASLTISGLQPEDEADYYCSAYTSSDTLVFGGGTKLTVL

SEQ ID NO: 277 - protein, 246 aa, synthetic construct
  REGION 1..246: note = Synthetic
EVQLVQSGAEVKKPGSSVKVSCKASGYTFTNYYMHWVRQAPGQGLEWMGAIMPSRGGTSYPQKFQGRVTMTGDTSTSTVYMELSSLRSEDTAVYYCARGEYYYDSSGYYYWGQGTLVTVSSGGGGSGGGGSGGGGSQSALTQPASVSGSPGQSIAISCTGTSSDIGHYDFVSWYQQHPGTAPKLIIYDNNNRPSGISNRFSGSKSDNMASLTISGLQPEDEADYYCSAYTSSDTVVFGGGTKLTVL

SEQ ID NO: 278 - protein, 253 aa, synthetic construct
  REGION 1..253: note = Synthetic
EVQLVQSGAEVKKPGSSVKVSCKASGYTFTNYYMHWVRQAPGQGLEWMGAIMPSRGGTSYPQKFQGRVTMTGDTSTSTVYMELSSLRSEDTAVYYCARGEYYYDSSGYYYWGQGTLVTVSSGGGGSGGGGSGGGGSQSALTQPASVSGSPGQSIVISCTGTSSDIGHYDFVSWYQQHPGTAPKLIIYDINNRPSGISNRFSGSKSDNMASLTISGLQPEDEADYYCSAYTSSDTVVFGGGTKLTVLAAAHHHH

SEQ ID NO: 279 - protein, 246 aa, synthetic construct
  REGION 1..246: note = Synthetic
EVQLVQSGAEVKKPGSSVKVSCKASGYTFTNYYMHWVRQAPGQGLEWMGAIMPSRGGTSYPQKFQGRVTMTGDTSTSTVYMELSNLRSEDTAVYYCARGEYYYDSSGYYYWGQGTLVTVSNGGGGSGGGGSGGGGSQSALTQPASVSGSPGQSIAISCTGTSSDIGHYDFVSWYQQHPGTAPKLIIYDINNRPSGISNRFSGSKSDNMASLTISGLQPEDEADYYCSAYTSSDTVVFGGGTKLTVL

SEQ ID NO: 280 - protein, 246 aa, synthetic construct
  REGION 1..246: note = Synthetic
EVQLVQSGAEVKKPGSSVKVSCKASGYTFTNYYMHWVRQAPGQGLEWMGAIMPSRGGTSYPQKFQGRVTMTGDTSTSTVYMELSSLRSEDTAVYYCARGEYYYDSSGYYYWGQGTLVTVSSGGGGSGGGGSGGGGSQSALTQPASVSGSPGQSIAISCTGTSSDIGHYDFVSWYQQHPGTAPKLIIYDINNRPSGISNRFSGSKSDNMASLTISGLQPEDEADYYCSAYTSSDTVVFGGGTKLTVL

SEQ ID NO: 281 - protein, 246 aa, synthetic construct
  REGION 1..246: note = Synthetic
EVQLVQSGAEVKEPGSSVKVSCKASGYTFTNYYMHWVRQAPGQGLEWMGAIMPSRGGTSYPQKFQGRVTMTGDISTSTVYMELSSLRSEDTAVYYCARGEYYYDSSGYYYWGQGTLVTVSSGGGGSGGGGSGGGGSQSALTQPASVSGSPGQSIAISCTGTSSDIGHYDFVSWYQQHPGTAPKLIIYDINNRPSGISNRFSGSKSDNMASLTISGLQPEDEADYYCSAYTSSDTVVFGGGTKLTVL

SEQ ID NO: 282 - protein, 246 aa, synthetic construct
  REGION 1..246: note = Synthetic
EVQLVQSGAEVKKPGSSVKVSCKASGYTFTNYYIHWVRQAPGQGLEWMGAIMPSRGGTSYPQKFQGRVTMTGDTSTSTVYMELNSLRSEDTAVYYCARGEYYYDSSGYYYWGQGTLVTVSSGGGGSGGGGSGGGGSQSALTQPASVSGSPGQSIAISCTGTSSDIGHYDFVSWYQQHPGTAPKLIIYDINNRPSGISNRFSGSKSDNMASLTISGLQPEDEADYYCSAYTSSDTVVFGGGTKLTVL

SEQ ID NO: 283 - protein, 246 aa, synthetic construct
  REGION 1..246: note = Synthetic
EVQLVQSGAEVKKPGSSVKVSCKASGYTFTNYYMHWVRQAPGQGLEWMGAIMPSRGGTSYPQKFQGRVTMTGDTSTSTVYMELSSLRSEDTAVYYCARGEYYYDSSGYYYWGQGTLVTVSSGGGGSGGGGSGGGGSQSALTQPASVSGSPGQSIAISCTGTSSDIGDYDFVSWYQQHPGTAPKLIIYDINNRPSGISNRFSGSKSDNMASLIISGLQPEDEADYYCSAYTSSDTLVFGGGTKLTVL

SEQ ID NO: 284 - protein, 246 aa, synthetic construct
  REGION 1..246: note = Synthetic
EVQLVQSGAEVKKPGSSVKVSCKASGYTFTNYYMHWVRQAPGQGLEWMGAIMPSRGGTSYPQKFQGRVTMTGDTSTSTVYMELSSLRSEDTAVYYCARGEYYYDSSGYYYWGQGTLVTVSSGGGGSGGGGSGGGGSQSALTQPASVSGSPGQSIAISCTGTSSDIGHYDFVSWYQQHPGTAPKLIIYDINNRPSGISNRFSGSKSDDMASLTISGLQPEDEADYYCSAYTSSDTVVFGGGTKLTVL

SEQ ID NO: 285 - protein, 246 aa, synthetic construct
  REGION 1..246: note = Synthetic
EVQLVQSGAEVKKPGSSVKVSCKASGYTFTNYYMHWVRQAPGQGLEWMGAIMPSRGGTSYPQKFQGRVTMTGDTSTSTVYMELSSLRSEDTAVYYCARGEYYYDSSGYYYWGPGTLVTVSSGGGGSGGGGSGGGGSQSALTQPASVSGSPGQSIAISCTGTSSDIGHYDFVSWYQQHPGTAPKLIIYDINNRPSGISNRFSGSKSDNMASLTISGLQPEDEADYYCSAYTSSDTVVFGGGTKLTVL

SEQ ID NO: 286 - protein, 246 aa, synthetic construct
  REGION 1..246: note = Synthetic
EVQLVQSGAEVKKPGSSVKVSCKASGYTFTNYYIHWVRQAPGQGLEWMGAIMPSRGGTSYPQKFQGRVTMTGDTSTSTVYMELSSLRSEDTAVYYCARGEYYYDSSGYYYWGQGTLVTVSSGGGGSGGGGSGGGGSQSALTQPASVSGSPGQSIAISCTGTSSDIGHYDFVSWYQQHPGTAPKLIIYDINNRASGISNRFSGSKSDNMASLTISGLQPEDEADYYCSAYTSSDTVVFGGGTKLTVL

SEQ ID NO: 287 - protein, 246 aa, synthetic construct
  REGION 1..246: note = Synthetic
EVQLVQSGAEVKKPGSSVKVSCKASGYTFTNYYMHWVRQAPGQGLEWMGAIMPSRGGTSYPQKFQGRVTMTGDTSTSTVYMELSSLRSEDTAVYYCARGEYYYDSSGYYYWGQGTLVTVSSGGGGSGGGGSGGGGSQSALTQPASVSGNPGQSIAISCTGTSSDIGHYDFVSWYQQHPGTAPKLIIYDINNRPSGISNRFSGSKSDNMASLTISGLQPEDEADYYCSAYTSSDTVVFGGGTKLTVL

SEQ ID NO: 288 - protein, 246 aa, synthetic construct
  REGION 1..246: note = Synthetic
EVQLVQSGAEVKKPGSSVKVSCKASGYTFTNYYMHWVRQAPGQGLEWMGAIMPSRGGTSYPQKFQGRVTMTGDTSTSTVYMELSSLRSEDTAMYYCARGEYYYDSSGYYYWGQGTLVTVSSGGGGSGGGGSGGGGSQSTLTQPASVSGSPGQSITISCTGTSSDIGHYDFVSWYQQHPGTAPKLIIYDINNRPSGISNRFSGSKSDNMASLTISGLQPEDEADYYCSAYTSSDTVVFGGGTKLTVL

SEQ ID NO: 289 - protein, 246 aa, synthetic construct
  REGION 1..246: note = Synthetic
EVQLVQSGAEVKKPGSSVKVSCKASGYTFTNYYMHWVRQAPGQGLEWMGAIMPSRGGTSYPQKFQGRVTMTGDTSTSTVYMELSSLRTEDTAVYYCARGEYYYDSSGYYYWGQGTLVTVSSGGGGSGGGGSGGVGSQSALTQPASVSGSPGQSIAISCTGTSSDIGHYDFVSWYQQHPGTAPKLIIYDINNRPSGISNRFSGSKSDNMASLTISGLQPEDEADYYCSAYTSSDTVVFGGGTKLTVL

SEQ ID NO: 290 - protein, 246 aa, synthetic construct
  REGION 1..246: note = Synthetic
EVQLVQSGAEVKKPGSSVKVSCKASGYTFTNYYMHWVRQAPGQGLEWMGAIMPSRGGTSYPQKFQGRVTMTGDTSTSTVYMELSNLRSEDTAVYYCARGEYYYDSSGYYYWGQGTLVTVSSGGGGSGGGGSGGGGSQSALTQPASVSGSPGQSIAISCTGTSSDIGHYDFVSWYQQHPGTAPKLIIYDINNRPSGISNRFSGSKSDNMASLTISGLQPEDEADYYCSAYTSSDTVVFGGGTKLTVL

SEQ ID NO: 291 - protein, 246 aa, synthetic construct
  REGION 1..246: note = Synthetic
EVQLVQSGAEVKKPGSSVKVSCKASGYTFTNYYMHWVRQAPGQGLEWMGAIMPSRGGTSYPQKFQGRVTMTGDTSTSTVYMELSSLRSEDTAVYYCARGEYYYDSSGYYYWGQGTLVNVSSGGGGSGGGGSGGGGSQSALTQPASVSGSPGQSIAISCTGTSSDIGHYDFVSWYQQHPGTAPKLIIYDINNRPSGISNRFSGSKSDNMASLTISGLQPEDEADYYCSAYTSSDTVVFGGGTKLTVL

SEQ ID NO: 292 - protein, 246 aa, synthetic construct
  REGION 1..246: note = Synthetic
EVQLVQSGAEVKKPGSSVKVSCKASGYTFSNYYMHWVRQAPGQGLEWIGAIMPSRGGTSYPQKFQGRVTMTGDTSTSTVYMELSSLRSEDTAVYYCARGEYYYDSSGYYYWGQGTLVTVSSGGGGSGGGGSGGGGSQSALTQPASVSGSPGQSIAISCTGTSSDIGHYDFVSWYQQHPGTAPKLIIYDINNRPSGISNRFSGSKSDNMASLTISGLQPEDEADYYCSAYTSSDTVVFGGGTKLTVL

SEQ ID NO: 293 - protein, 246 aa, synthetic construct
  REGION 1..246: note = Synthetic
EVQLVQSGAEVKKPGSSVKVSCKATGYTFTNYYMHWVRQAPGQGLEWMGAIMPSRGGTSYPQKFQGRVTMTGDTSTSTVYMELSSLRSEDTAVYYCARGEYYYDSSGYYYWGQGTLVTVSSGGGGSGGGGSGGGGSQSALTQPASVSGSPGQSIAISCTGTSSDIGHYDFVSWYQQHPGTAPKLIIYDINNRPSGISNRFSGSKSDNMASLTISGLQPEDEADYYCSAYTSSDTVVFGGGTKLTVL

SEQ ID NO: 294 - protein, 246 aa, synthetic construct
  REGION 1..246: note = Synthetic
EVQLVQSGAEVKKPGSSVKVSCKASGYTFTNYYMHWVRQAPGQGLEWMGAIMPSRGGTSYPQKFQGRVTMTGDTSTSTVYMELSSLRSEDTAVYYCARGEYYYDSSGYYYWGQGTPVTVSSGGGGSGGGGSGGGGSQSALTQPASVSGSPGQSIAISCTGTSSDIGDYDFVSWYQQHPGTAPKLIIYDINNRPSGISNRFSGSKSDNMASLTISGLQPEDEADYYCSAYTSSDTVVFGGGTKLTVL

SEQ ID NO: 295 - protein, 246 aa, synthetic construct
  REGION 1..246: note = Synthetic
EVQLVQSGAEVKKPGASVKVSCKASGYTFTNYYMHWVRQAPGQGLEWMGAIMPSRGGTSYPQKFQGRVTITADKSTSTAYMELSSLRSEDTAVYYCARGEYYYDSSGYYYWGQGTLVTVSSGGGGSGGGGSGGGGSQSVLTQPPSVSGAPGQRVTISCTGTSSDIGHYDFVSWYQQLPGTAPKLLIYDINNRPSGVPDRFSGSKSGTSASLAITGLQAEDEADYYCSAYTSSDTLVFGGGTKLTVL

SEQ ID NO: 296 - protein, 253 aa, synthetic construct
  REGION 1..253: note = Synthetic
EVQLVQSGGGVVQPGRSLRLSCAASGFTFSSYAMHWVRQAPGKGLEWVAVISYDGSNKYYADSVKGRFTISRDNSKNTLYLQMNSLRAEDTAVYYCARDLGYSSSWYYYYYGMDVWGQGTTVTVSSGGGGSGGGGSGGGGSDVVMTQSPLSLPVTPGEPASISCRSSQSLLHSNGYNYLDWYLQKPGQSPQLLIYLGSSRASGVPDRFSGSGSGTDFTLKISRVEAEDVGVYYCMQALQTPRTFGQGTKLEIK

SEQ ID NO: 297 - protein, 241 aa, synthetic construct
  REGION 1..241: note = Synthetic
QVQLQESGPGLVKPSETLSLTCTVSGVSISSRSDHWGWVRQPPGKGLEWIGSISYSGSTYYNPSLKSRVTISVDTSKNQLSLKLSSVTAADTAVYYCARESHPAAALVGWGQGTLVTVSSGGGGSGGGGSGGGGSDIQMTQSPSSLSASVGDRVTITCQASQDISDYLNWYQQKPGKAPQILIYDASNLETGVPSRFSGSGSGTDFTFTISNLQPEDVATYYCQQYEDLPSFGGGTKVEIK

SEQ ID NO: 298 - protein, 234 aa, synthetic construct
  REGION 1..234: note = Synthetic
EVQLVQSGGGLVQPGGSLRLSCAASGFTFSSYAMSWVRQAPGKGLEWVSAISGSGGSTHYADSVKGRFTISRDNSKNTLYLQMNSLRAEDTAVYYCATPAFWGQGTLVTVSSGGGGSGGGGSGGGGSDIQLTQSPSSLSASVGDRVTITCRASQSISSYLNWYQQKPGKAPKALIYDASNLETGVPSRFSGSGSGTDFTLTIISLQPEDFATYFCLQDYIYPWTFGQGTKVEFK

SEQ ID NO: 299 - protein, 243 aa, synthetic construct
  REGION 1..243: note = Synthetic
QVQLQQWGPGLVKPSETLSLTCTVSGGSISSYYWSWIRQPPGKGLEWIGEINHSGSTNYNPSLKSRVTISVDTSKNQFSLKLSSVTAADTAVYYCAGGSNLDWFDPWGQGTLVTVSSGGGGSGGGGSGGGGSQSVLTQPPSVSGAPGQRVTISCTGGSSNVGAGYDVHWYQQLPGTVPKLLIYDNTNRPSGVPDRFSASKSGTSASLVITGLQAEDEGDYYCQSYDSSLRASVFGGGTMLTVL

SEQ ID NO: 300 - protein, 244 aa, synthetic construct
  REGION 1..244: note = Synthetic
QVQLQQWGAGLLKPSETLSLTCAVYGGSFSGYYWSWIRQPPGKGLEWIGEINHSGSTNYNPSLKSRVTISVDTSKNQFSLELSSVTAADTAVYYCARADRRFGELRYWGQGTLVTVSSGGGGSGGGGSGGGGSNFMLTQPHSVSESPGKTVTISCTGSSGSIASSYVQWYQQRPGSAPTTVIYADNQRPSGVPDRFSGSVDSSSNSASLTISGLKTEDEADYYCQSFDSSLYMIFGGGTKLTVL

SEQ ID NO: 301 - protein, 247 aa, synthetic construct
  REGION 1..247: note = Synthetic
QVQLVQSGGGLVKPGGSLRLSCAASGFTFSSYDLHWVRQVPGKGLEWVSLISYDGSNKYYADSVKGRFTISRDNAENSLYLQMNSLRAEDTAVYYCAREPITGTSDLFDYWGQGTLVTVSSGGGGSGGGGSGGGGSQSVLTQPPSVSGAPGQRVTISCTGSRSNIGAGYDVHWYQHLPGTAPKLLIYDNSNRPSGVSDRFSGSKSGTSASLAITGLQAEDEADYYCQSFDSSLRGVVFGGGTRLTVL

SEQ ID NO: 302 - protein, 243 aa, synthetic construct
  REGION 1..243: note = Synthetic
QVQLQESGPGLVKPSGTLSLTCAVSGGSISSSNWWSWVRQPPGKGLEWIGEIYHSGSTNYNPSLKSRVTISVDKSKNQFSLKLSSVTAADTAVYYCVREGGLREEHWGQGTLVTVSSGGGGSGGGGSGGGGSSYELTQPPSASGTPGQRVTISCSGSSSNIGSNTVNWYQQLPGTAPKLLIYSNNQRPSGVPDRFSGSKSGTSASLAISGLQSEDEADYYCAAWDDSLNGLWVFGGGTKLTVL

SEQ ID NO: 303 - protein, 244 aa, synthetic construct
  REGION 1..244: note = Synthetic
QVQLQQWGAGLLKPSETLSLTCAVYGGSFSGYYWSWIRQPPGKGLEWIGEINHSGSTNYNPSLKSRVTISVDTSKNQFSLKLSSVTAADTAVYYCARGTDTAMADYWGQGTLVTVSSGGGGSGGGGSGGGGSDVVMTQSPLSLPVTPGEPASISCRSSQRLLHSNGYNYVDWYLQKPGQSPQLLIYLGSNRASGVPDRFSGSGSGTDFTLKISRVEAEDVGVYYCMQGTHWPWTFGQGTKVEIK

SEQ ID NO: 304 - protein, 242 aa, synthetic construct
  REGION 1..242: note = Synthetic
QVQLQQSGPGLVKPSQTLSLTCAISGDSVSGNSATWNWIRQSPSRGLEWLGRTYYRSKWNHDYAESVKSRITINPDTSKNQFSLQLNSVTPEDTAVYYCARDSKSAFDIWGQGTMVTVSSGGGGSGGGGSGGGGSDIQMTQSPPSLSASVGDRVTITCQASQDINNYLNWYHQKPGKAPELLIYDASQLETGVPSRFSGSGSGTEFTFIISSLQPEDTGTYYCQQYDWLPLSYGGGTKVEIK

SEQ ID NO: 305 - protein, 239 aa, synthetic construct
  REGION 1..239: note = Synthetic
QLQLQESGPGLVKPSETLSLTCSVFGVSITSGSWWSWVRQSPGKELEWIGEIYHNGNTNYNPSLKSRVTISVDTSKNQFSLKLSSVTAADTAVYYCVSGFDYWGQGTLVTVSSGGGGSGGGGSGGGGSNFMLTQPHSVSGSPGKTITISCTRSSGNFASTYVQWYQQRPGSSPAIVIYDDDQRPSGVPDRFSGSIDRSSNSASLTISGLETEDEADYYCQSYDSSNFWVFGGGTKLTVL

SEQ ID NO: 306 - protein, 241 aa, synthetic construct
  REGION 1..241: note = Synthetic
QVQLQESGPGLVKPSETLSLTCTVSGVSISSRSDHWGWVRQPPGKGLEWIGSISYSGSTYYNPSLKSRVTISVDTSKNQLSLKLSSVTAADTAVYYCARESHPAAALVGWGQGTLVTVSSGGGGSGGGGSGGGGSEIVLTQSPSSLSASVGDRVTITCQASQDINNYLNWYQQKPGKAPKLLIYDASNLETGVPSKFSGSGSGTDFTFTISSLQPEDIATYYCQQYANLPSFGQGTKLEIK

SEQ ID NO: 307 - protein, 253 aa, synthetic construct
  REGION 1..253: note = Synthetic
EVQLVQSGAEVKKPGSSVKVSCKASGYTFTSYGISWVRQAPGQGLEWMGWISAYNGNTNYAQKLQGRVTMTTDTSTSTAYMELSSLRSEDTAVYYCARAPDYGDSSNYYYYYMDVWGKGTTVTVSSGGGGSGGGGSGGGGSEIVLTQSPLSLPVTPGEPASISCRSSQSLLHSNGYNYLDWYLQKPGQSPQLLIYLGSNRASGVPDRFSGSGSGTDFTLSISRVEAEDVGVYYCMQALQTPPTFGQGTKVEIK

SEQ ID NO: 308 - protein, 249 aa, synthetic construct
  REGION 1..249: note = Synthetic
QVQLVQSGAEVKKPGASVKVSCKASGYTFTSYGISWVRQAPGQGLEWMGWISAYNGNTNYAQKLQGRVTMTTDTSTSTAYMELRSLRSDDTAVYYCARDTSGDYSSGWYLGVPFDYWGQGTLVTVSSGGGGSGGGGSGGGGSDIQLTQSPSSLSASVGDRVTVTCQASQDIGHNLNWYQQRPGKAPQLLIYDASNLETGVPSRFSGSGSGTQFTFTISSLQPEDIATYYCQQYDFLPPDFGPGTKVEIK

SEQ ID NO: 309 - protein, 482 aa, synthetic construct
  REGION 1..482: note = Synthetic
QVQLQQWGAGLLKPSETLSLTCAVYGGSFSGYYWSWIRQPPGKGLEWIGEINHSGSTNYNPSLKSRATISVDTSKNQFSLKLSSVTAADTAVYYCATSPGGYSGGYFQHWGQGTLVTVSSGGGGSGGGGSGGGGSDVVMTQSPLSLPVTPGEPASISCRSSQSLLHSNGYNYLDWYLQKPGQSPQLLIYLGSNRDSGVPDRFSGSGSGTDFTLKISRVEAEDVGVYYCMQGTHWPWTFGQGTKVEIKNSGAGTAAATHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLCVSKLTVDKSRWQQGNVFSCSVMHEALHNHYTQKSLSLSPGK

SEQ ID NO: 310 - protein, 243 aa, synthetic construct
  REGION 1..243: note = Synthetic
QVQLQESGPGLVKPSETLSLTCTVSGGSISSSSYYWGWIRQPPGKGLEWIGSIYYSGSTYYNPSLKSRVTISVDTSKNQFSLKLSSVTAADTAVYYCAGDILTGYALDYWGQGTLVTVSSGGGGSGGGGSGGGGSSYELTQPPSMSVSPGQTARITCSGDALPKQFAFWYQQKPGQAPVLVIYKDTERPSGIPERFSGSSSGTTVTLTITGVQAEDEADYYCQSPDSSGTVEVFGGGTKLTVL

SEQ ID NO: 311 - protein, 247 aa, synthetic construct
  REGION 1..247: note = Synthetic
QVQLQQWGAGLLKPSETLSLTCAVYGGSFSGYYWSWIRQPPGKGLEWIGEINHFGSTNYNPSLKSRATISVDTSKNQFSLKLSSVTAADTAVYYCATSPGGYSGGYFQHWGQGTLVTVSSGGGGSGGGGSGGGGSDVVMTQSPLSLPVTPGEPASISCRSSQSLLHSNGYNYLDWYLQKPGQSPQLLIYLGSNRDSGVPDRFSGSGSGTDFTLKISRVEAEDVGVYYCMQGTHWPWTFGQGTKVEIK

SEQ ID NO: 312 - protein, 247 aa, synthetic construct
  REGION 1..247: note = Synthetic
QVQLQQWGAGLLKPSETLSLTCAVYGGSFSGYYWSWIRQPPGKGLEWIGEINHFGSTNYNPSLKSRATISVDTSKNQFSLKLSSVTAADTAVYYCATSPGGYSGGYFQHWGQGTLVTVSSGGGGSGGGGSGGGGSDVVMTQSPLSLPVTPGESVSISCRSSQSLLHSNGYNYLDWYLQKPGQSPQLLIYLGSNRDSGVPDRFSGSGSGTDFTLKISRVEAEDVGVYYCMQGTHWPWTFGQGTKVEIK

SEQ ID NO: 313 - protein, 247 aa, synthetic construct
  REGION 1..247: note = Synthetic
QVQLQQWGAGMLKPSETLSLTCAVYGGSFSGYYWSWIRQPPGKGLEWIGEINHFGSTNYNPSLKSRATISVDTSKNQFSLKLSSVTAADTAVYYCATSPGGYSGGYFQHWGQGTLVTVSSGGGGSGGGGSGGGGSDVVMTQSPLSLPVTPGEPASISCRSSQSLLHSNGYNYLDWYLQKPGQSPQLLIYLGSNRDSGVPDRFSGSGSGTDFTLKISRVEAEDVGVYYCMQGTHWPWTFGQGTKVEIK

SEQ ID NO: 314 - protein, 255 aa, synthetic construct
  REGION 1..255: note = Synthetic
QVQLQQWGAGLLKPSETLSLTCAVYGGSFSGYYWSWIRQPPGKGLEWIGEINHFGSTNYNPSLKSRATISVDTSKNQFSLKLSSVTAADTAVYYCATSPGGYSGGYFQHWGQGTLVTVSSGGGGSGEGGSGGGGSDVVMTQSPLSLPVTPGEPASISCRSSQSLLHSNGYNYLDWYLQKPGQSPQLLIYLGSNRDSGVPDRFSGSGSGTDFTLKISRVEAEDVGVYYCMQGTHWPWTFGQGTKVEIKAAAHHHHH

SEQ ID NO: 315 - protein, 247 aa, synthetic construct
  REGION 1..247: note = Synthetic
EVQLVESGGGLVQPGGSLRLSCAASGGSFSGYYWSWVRQAPGKGLEWVSEINHSGSTNYNPSLKSRFTISRDNSKNTLYLQMNSLRAEDTAVYYCARSPGGYSGGYFQHWGQGTLVTVSSGGGGSGGGGSGGGGSDIQMTQSPSSLSASVGDRVTITCRSSQSLLHSNGYNYLDWYQQKPGKAPKLLIYLGSNRDSGVPSRFSGSGSGTDFTLTISSLQPEDFATYYCMQGTHWPWTFGQGTKVEIK

SEQ ID NO: 316 - protein, 247 aa, synthetic construct
  REGION 1..247: note = Synthetic
QVQLQQSGAEVKKPGSSVKVSCKASGGTFSSYAISWVRQAPGQGLEWMGGFDPEDGETIYAQKFQGRVTMTEDTSTDTAYMELSSLRSEDTAVYYCATDLRIPYYYDNPWGQGTLVTVSSGGGGSGGGGSGGGGSEIVLTQSPLSLPVTPGEPASISCRSSQSLLHSNGYNYLNWYLQKPGQSPQLLIYLGSDRASGVPDRFSGSGSGTDFTLKISRVEAEDVGVYYCMQALQTPTTFGGGTKVEIK

SEQ ID NO: 317 - protein, 245 aa, synthetic construct
  REGION 1..245: note = Synthetic
QVQLVQSGGGVVQPGRSLRLSCAASGFTFSSYAMHWVRQAPGKGLEWVAVISYDGSNKYYADSVKGRFTISRDNSKNTLYLQMNSLRAEDTAVYYCARSLYYSHFDYWGQGTLVTVSSGGGGSGGGGSGGGGSEIVMTQSPLSLPVTPGEPASISCRSSQSLLHSNGYNYLDWYLQKPGQSPQLLMYLVSNRASGVPERFSGSGSGTDFTLKISRVEAEDVGVYYCMQTLQTPLSFGQGTKLEIK

SEQ ID NO: 318 - protein, 245 aa, synthetic construct
  REGION 1..245: note = Synthetic
EVQLVETGPGLVKPSGTLSLTCAVSGGSISSSNWWSWVRQPPGKGLEWIGEIYHSGSTNYNPSLKSRVTISVDKSKNQFSLKLSSVTAADTAVYYCAREGPLSSSGPGAFDIWGQGTMVTVSSGGGGSGGGGSGGGGSEIVLTQSPATLSLSPGERATLSCRASQSVSYHLAWYQQKPGQAPRLLIYDTSNRASGIPARFSGSGSGTDFTLTINSLEPEDFAVYYCQQRYDWPLTFGGGTKVEIK

SEQ ID NO: 319 - protein, 236 aa, synthetic construct
  REGION 1..236: note = Synthetic
QVQLQESGGGVVQPGRSLRLSCAASGFTFSNYGMHWVRQAPGKGLEWVAVISYDGTNKYYADSVKGRFTISRDNSKNTVYLQMNSLRAEDTAVYYCAKDGFDIWGQGTMVTVSSGGGGSGGGGSGGGGSDIQMTQSPSFLSASVGDRVTITCRASQSISSWLAWYQQKPGKAPKLLIYDASRLEDGVPSRFSGTGFGTDFTFTITTLQPDDIATYYCQQYDDLPYTFGQGTTVDIK

SEQ ID NO: 320 - protein, 247 aa, synthetic construct
  REGION 1..247: note = Synthetic
EVQLVQSGGGVVQPGRSLRLSCAASGFTFSSYGMHWVRQAPGKGLEWVAVISYDGSNKYYADSVKGRFTISRDNSKNTLYLQMNSLRAEDTAVYYCARDVYGDYGAFDYWGQGTLVTVSSGGGGSGGGGSGGGGSDVVMTQSPVSLPVTLGQPASISCKSSQSLLYFNGNTYLSWFQQRPGQSPRRLFYQVSNRDSGVPDRFSGSGSDTDFTLTISRVEAEDVGVYFCMQGTQWPPTFGQGTKVEIK

SEQ ID NO: 321 - protein, 244 aa, synthetic construct
  REGION 1..244: note = Synthetic
QLQLQESGGGVVQPGRSLRLSCAASGFTFSSYAMHWVRQAPGKGLEWVAVISYDGGNKYYADSVKGRFTISRDNSKNTLYLQMNSLRAEDTAVYYCAKSVAPPMDVWGKGTTVTVSSGGGGSGGGGSGGGGSDVVMTQSPLSLPVTPGEPASISCRSSQSLLHSNGYNYLDWYLQKPGQSPHLLIYLGSNRASGVPDRFSGSGSGTDFTLKISRVEAEDVGVYFCMQALRTPYTFGQGTKLEIK

SEQ ID NO: 322 - protein, 248 aa, synthetic construct
  REGION 1..248: note = Synthetic
QVQLQQWGAGLLKPSETLSLTCAVYGESFSGYYWSWIRQPPGKGLEWIGEINHSGSTNYNPSLKSRVTISVDTSKNQFSLKLSSVTAADTAVYYCARGPAGSSSSGYFDYWGQGTLVTVSSGGGGSGGGGSGGGGSDVVMTQSPLSLPVTLGQPASISCRSSQSLVHSNGYNYLDWYLQKPGQSPQLLIYLGSNRASGVPDRFSGSGSGTDFTLKISRVEAEDVGVYYCLQGSHWPWTFGQGTKVEIK

SEQ ID NO: 323 - protein, 237 aa, synthetic construct
  REGION 1..237: note = Synthetic
QVQLQESGPGLVKPSQTLSLTCTVSGGSISSGGYYWTWIRQHPGQGLEWIGFISWSGTTYYNPSLKNRVTISADTSKNHFSLNLTSVTAADTAVYYCARGSGRLVWGQGTLVTVSSGGGGSGGGGSGGGGSETTLTQSPATLSVSPGERATLSCRASQSVSSNLAWYQQKPGQAPRLLIYGASSGATGIPDRFSGSGSGTDFTLTISRLEPEDFAVYYCQLYGSSLAFGGGTKVEIK

SEQ ID NO: 324 - protein, 248 aa, synthetic construct
  REGION 1..248: note = Synthetic
EVQLVQSGAEVKKPGASVKVSCKASGYTFTGYYMHWVRQAPGQGLEWMGIINPSGGSTSYAQKFQGRVTMTRDTSTSTVYMELSSLRSEDTAVYYCARADTAMGDAFDIWGQGTMVTVSSGGGGSGGGGSGGGGSDIVMTHTPLSLPVTPGEPASISCRSSQTLFDSDDGKTYLDWYLQKPGQSPQLLMYTTSSRASGVPDRFSGSGSGTDFTLKISRVEAEDVGVYYCMQRLQFPLTFGQGTRLEFK

SEQ ID NO: 325 - protein, 245 aa, synthetic construct
  REGION 1..245: note = Synthetic
EVQLVQSGGGVVQPGRSLRLSCAASGFTFSSYAMHWVRQAPGKGLEWVAVISYDGSNKYYADSVKGRFTISRDNSKNTLYLQMNSLRAEDTAVYYCARSIGIGAFDIWGQGTMVTVSSGGGGSGGGGSGGGGSDVVMTQSPLSLPVTPGEPASISCRATQSLLHGNGHNYLDWYLQKPGQSPQLLIYLGSNRASGVPDRFSGSGSGTDFTLKISRVEAEDVGVYYCMQTLETPVTFGPGTKVDIK

SEQ ID NO: 326 - protein, 248 aa, synthetic construct
  REGION 1..248: note = Synthetic
QVQLQQWGAGLLKPSETLSLTCTIYGGSFSGFYWSWIRQPPGKGLEWIGEINHSGSTNYNPSLKSRVTISVDTSKNQFSLKLSSVTAADTAIYYCARGPAGSTSSGYFDHWGQGTLVTVSSGGGGSGGGGSGGGGSDVVMTQSPLSLPVTPGEPASISCRSSQSLLHSNGYNYLDWYLQKPGQSPQLLIYLGSNRASGVPDRFSGSGSGTDFTLKISRVEAEDVGVYYCMQGTHWPWTFGQGTKVEIK

SEQ ID NO: 327 - protein, 244 aa, synthetic construct
  REGION 1..244: note = Synthetic
QVQLQQWGAGLLKPSETLSLTCAVYGGSLSGYYWSWIRQPPGKGLEWIGEINHSGSTNYNPSLKSRVTISVDTSKNQFSLKLSSVTAADTAVYYCARGSSSYYMDVWGKGTTVTVSSGGGGSGGGGSGGGGSDVVMTQSPLSLPVTPGEPASISCRSSQSLLHSNGYNYLDWYLQKPGQSPQLLIYLGSNRASGVPDRFSGSGSGTDFTLKISRVEAEDVGVYYCLQGTHWPWTFGQGTKVEIK

SEQ ID NO: 328 - protein, 243 aa, synthetic construct
  REGION 1..243: note = Synthetic
QVQLQQWGAGLLKPSETLSLTCAVYGGSFSGYYWSWIRQPPGKGLEWIGEINHSGSTNYNPSLKSRVTISVDTSKNQFSLKLSSVTAADTAVYYCARGGSAYFQHWGQGTLVTVSSGGGGSGGGGSGGGGSDVVMTQSPLSLPVTLGQPASISCRSSQSLLHSNGNNYLDWYLQKPGQSPQLLIYLGSNRASGVPDRFSGSGSGTDFTLKISRVEAEDVGIYYCMQGTHWPWTFGQGTKVEIE

SEQ ID NO: 329 - protein, 248 aa, synthetic construct
  REGION 1..248: note = Synthetic
QVQLQQWGAGLLKPSETLSLTCAVYGGSFSGYYWSWIRQPPGKGLEWIGEINHSGSTNYNPSLKSRVTISEDASKKQFSLTLTSVTAADTAVYYCARGPAGTGSSGYFDYWGQGTLVTVSSGGGGSGGGGSGGGGSEIVLTQSPLSLPVTPGEPASISCRSSQSLLHSNGYNYLDWYLQKPGQSPQLLIYLASNRASGVPDRFSGSGSGTDFTLKISRVEAEDVGVYYCMQGTHWPWTFGQGTKVEVK

SEQ ID NO: 330 - protein, 9 aa, synthetic construct
  REGION 1..9: note = Synthetic
NSGAGTAAA

SEQ ID NO: 331 - protein, 23 aa, synthetic construct
  REGION 1..23: note = Synthetic
NSGAGTSGSGASGEGSGSKLAAA

SEQ ID NO: 332 - protein, 8 aa, synthetic construct
  REGION 1..8: note = Synthetic
GGGGSAAA

SEQ ID NO: 333 - protein, 15 aa, synthetic construct
  REGION 1..15: note = Synthetic
GGGGSGGGGSGGGGS

SEQ ID NO: 334 - protein, 9 aa, synthetic construct
  REGION 1..9: note = Synthetic
AAAHHHHHH

SEQ ID NO: 335 - DNA, 648 bp, synthetic construct
  misc_feature 1..648: note = Synthetic
  source 1..648: mol_type = other DNA
cagtctgccctgactcagcctgcctccgtgtctgggtctcctggacagtcgatcgccatttcctgcactggaaccagcagtgacattggtcattatgactttgtctcctggtaccaacagcacccaggcacagcccccaaactcataatttatgatatcaataatcggccctcagggatttctaatcgcttctctggctccaagtctgacaatatggcctccctgaccatctctgggctccagcctgaggacgaggctgattattactgcagtgcatatacaagcagcgacactctggtcttcggcggagggaccaagttgaccgtcctcagtcagcccaaggctgccccctcggtcactctgttcccaccctcctctgaggagcttcaagccaacaaggccacactggtgtgtctcataagtgacttctacccgggagccgtgacagtggcctggaaggcagatagcagccccgtcaaggcgggagtggagaccaccacaccctccaaacaaagcaacaacaagtacgcggccagcagctacctgagcctgacgcctgagcagtggaagtcccacaaaagctacagctgccaggtcacgcatgaagggagcaccgtggagaagacagtggcccctacagaatgttca

SEQ ID NO: 336 - DNA, 672 bp, synthetic construct
  misc_feature 1..672: note = Synthetic
  source 1..672: mol_type = other DNA
gaggtccagctggtacagtctggggctgaggtgaagaagcctgggtcctcagtgaaggtttcctgcaaggcatctggatacaccttcaccaactactatatgcactgggtgcgacaggcccctggacaagggcttgagtggatgggggcaatcatgcctagtcgtggtggcacaagttacccacagaagttccagggcagagtcaccatgaccggggacacgtccacgagcacagtctacatggagctgagcagcctgagatctgaggacacggccgtgtattactgtgcgagaggggagtattactatgatagtagtggttattactactggggccagggcaccctggtcaccgtctcaagcgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtccacaccttcccggctgtcctacagtcctcaggactctactccctcagcagcgtagtgaccgtgccctccagcagcttgggcacccagacctacatctgcaacgtgaatcacaagcccagcaacaccaaggtggacaagaaagttgagcccaaatcttgt

SEQ ID NO: 337 - DNA, 1440 bp, synthetic construct
  misc_feature 1..1440: note = Synthetic
  source 1..1440: mol_type = other DNA
gaagtgcagctggtgcagagcggggcagaagtgaaaaagcctgggtcaagcgtgaaggtctcctgtaaagcaagcggatacacattcacaaactactatatgcactgggtgcggcaggcccccggacagggcctggagtggatgggcgctatcatgccttcccgaggcgggacttcttacccacagaagttccagggaagagtgaccatgacaggcgacactagcacctccacagtctatatggagctgagcagcctgaggagcgaagacactgccgtgtactattgcgctcgcggagaatactattacgattctagtggctattactattgggggcagggaacactggtgactgtctcaagcggaggaggaggaagtggcggaggaggctccggaggaggcgggtctcagagtgcactgacccagccagcatcagtgagcggcagccccggccagtctatcgcaattagttgtactgggacctcctctgacatcggacactacgatttcgtctcttggtatcagcagcaccccggcaccgctcctaagctgatcatctacgacatcaacaatcggcccagcggcatttccaacagattttctgggagtaaatcagataatatggcctcactgacaattagcggcctccagcctgaggacgaagctgattactattgctccgcatacactagttcagataccctggtgtttggaggcgggaccaaactgacagtcctgaacagcggcgcgggcaccgcggccgcgactcacacatgcccaccgtgcccagcacctgaagccgcggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatgccgggatgagctgaccaagaaccaggtcagcctgtggtgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 338 - DNA, 642 bp, synthetic construct
  misc_feature 1..642: note = Synthetic
  source 1..642: mol_type = other DNA
gacatccagatgacccagtctccttccacattgtctgcatctgtaggagacagagtcacactctcttgccgggccggtcaggctattagtagttggttggcctggtatcaacagaaaccaggtaaagccccaaagcttctgatctataaggcatctaatttagaaagtggagtcccatcaaggttcagcggcggtggatctggggcagaattcactctcaccatcagcagcctgcagcctgatgattttgcaacttattactgccaacagtatcagagctacccttacacttttggccaggggaccaagctggagatcagacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 339 - DNA, 666 bp, synthetic construct
  misc_feature 1..666: note = Synthetic
  source 1..666: mol_type = other DNA
gaggtgcagctggtgcagtctgggactgaggtgaagaagcctggggcctcagtgaaggtttcctgcaaggcttctggatacaccttcactacctatgctatgcattgggtgcgccaggcccccggacaaagccttgagtggatgggatggatcaacactggcaatggtaacacaaaatattcacagaacttccagggcagagtcaccatgaccagggacacgtccatcagcacagcctacatggagctgagcaggctgagatctgacgacacggccgtgtattactgtgcgagagatctcgggcaactggaacgactctacttctggggccagggcaccctggtcaccgtctcaagcgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtccacaccttcccggctgtcctacagtcctcaggactctactccctcagcagcgtagtgaccgtgccctccagcagcttgggcacccagacctacatctgcaacgtgaatcacaagcccagcaacaccaaggtggacaagaaagttgagcccaaatcttgt

SEQ ID NO: 340 - DNA, 1428 bp, synthetic construct
  misc_feature 1..1428: note = Synthetic
  source 1..1428: mol_type = other DNA
gaggtgcagctggtgcagtctgggactgaggtgaagaagcctggggcctcagtgaaggtttcctgcaaggcttctggatacaccttcactacctatgctatgcattgggtgcgccaggcccccggacaaagccttgagtggatgggatggatcaacactggcaatggtaacacaaaatattcacagaacttccagggcagagtcaccatgaccagggacacgtccatcagcacagcctacatggagctgagcaggctgagatctgacgacacggccgtgtattactgtgcgagagatctcgggcaactggaacgactctacttctggggccagggcaccctggtcaccgtctcaagcggaggaggaggatctggcggaggaggcagtggaggaggagggtcacttgacatccagatgacccagtctccttccacattgtctgcatctgtaggagacagagtcacactctcttgccgggccggtcaggctattagtagttggttggcctggtatcaacagaaaccaggtaaagccccaaagcttctgatctataaggcatctaatttagaaagtggagtcccatcaaggttcagcggcggtggatctggggcagaattcactctcaccatcagcagcctgcagcctgatgattttgcaacttattactgccaacagtatcagagctacccttacacttttggccaggggaccaagctggagatcagaaacagcggcgcgggcaccgcggccgcgactcacacatgcccaccgtgcccagcacctgaagccgcggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatgccgggatgagctgaccaagaaccaggtcagcctgtggtgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 341 - DNA, 642 bp, synthetic construct
  misc_feature 1..642: note = Synthetic
  source 1..642: mol_type = other DNA
gacatccagttgacccagtctccatcctccctgtctgcatctgtaggagacagagtcaccatcacttgccaggcgagtcaggacattggcaactatttaaattggtatcagcttaaaccagggaaagcccctaagctcctgatctacgatgcatccaatttggaaacaggggtcccatcaaggttcagtggaagtggatctgggacagattttactttcaccatcagcagcctgcagcctgaagatattgcaacatattactgtctacaactttatgattaccccctcactttcggcggagggaccaaggtggagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 342 - DNA, 651 bp, synthetic construct
  misc_feature 1..651: note = Synthetic
  source 1..651: mol_type = other DNA
cacgtgcagctggtggagactgggggaggcttggtgcagcctggggggtccctgagactctcctgtgcagcctctggattcacctttagcagctatgccatgagctgggtccgccaggctccagggaaggggctggagtgggtctcagctattagtggtagtggtggtagcacatactacgcagactccgtgaagggccggttcaccatctccagagacaattccaagaacacgctgtatctgcaaatgaacagcctgagagccgaggacacggccgtatattactgtgcgagagatctcggggattattggggccagggaaccctggtcaccgtctcaagcgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtccacaccttcccggctgtcctacagtcctcaggactctactccctcagcagcgtagtgaccgtgccctccagcagcttgggcacccagacctacatctgcaacgtgaatcacaagcccagcaacaccaaggtggacaagaaagttgagcccaaatcttgt

SEQ ID NO: 343 - DNA, 1452 bp, synthetic construct
  misc_feature 1..1452: note = Synthetic
  source 1..1452: mol_type = other DNA
caggtccagctgcaggagtccgggccagggctggtgaaaccaagcgaaacactgagtctgacatgtaccgtgagtggggggtccattaacaatagtaactactattggtcatggatcagacagagccctggaagaggcctggagtggatcggcgggatctacttcagcggcaccacatactataacccatcactgcagagccgggtgactatctccattgacacctctaagaatcagttcagcctgaagctgagcagcgtgaccgccgctgatacagccatctactattgcgtccggcagatgaattactatcacctgggctctagtgtggggttcgacccctggggacagggagcactggccaccgtgtcaagcgtctcctctggaggaggaggcagcggcggaggaggctctggaggaggcgggagtgatgtggtcatgacacagagcccagctactctgtctgtgagtcccggcgaaagggccacactgagctgtcgcgcttcacagagcgtcagttcaaacctggcatggtaccagcagaagccaggacaggcaccttccctgctgatctatgaggcttctacacgagcaactggcattcctgctagattctccggctctgggagtggaaccgactttactctgaccatcagctccctgcagagcgaagattttgcaatctactattgtcagcagtataacgattggctgtggaccttcgggcaggggactaaagtggagattcggaacagcggcgcgggcaccgcggccgcgactcacacatgcccaccgtgcccagcacctgaagccgcggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatgccgggatgagctgaccaagaaccaggtcagcctgtggtgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 344 - DNA, 648 bp, synthetic construct
  misc_feature 1..648: note = Synthetic
  source 1..648: mol_type = other DNA
aattttatgctgactcagccccactctgtgtcggagtctccggggaagacggtaaccatctcctgcacccgcagcagtggcagcattgccagcaactatgtgcagtggtaccagcagcgcccgggcagttcccccaccacggtcatttttgacgacaatcaaagacccactggtgtccctgatcgcttctctgccgccatcgacacctcctccagttctgcctccctcaccatctctggactgacggctgaggacgaggccgattactattgtcagtcgtctcatagcaccgctgtcgtctttggcggagggaccaagctgaccgtcctaagtcagcccaaggctgccccctcggtcactctgttcccgccctcctctgaggagcttcaagccaacaaggccacactggtgtgtctcataagtgacttctacccgggagccgtgacagtggcctggaaggcagatagcagccccgtcaaggcgggagtggagaccaccacaccctccaaacaaagcaacaacaagtacgcggccagcagctacctgagcctgacgcctgagcagtggaagtcccacaaaagctacagctgccaggtcacgcatgaagggagcaccgtggagaagacagtggcccctacagaatgttca

SEQ ID NO: 345 - DNA, 660 bp, synthetic construct
  misc_feature 1..660: note = Synthetic
  source 1..660: mol_type = other DNA
caggtgcagctacagcagtggggcgcaggactgttgaagccttcggagaccctgtccctcacctgcgctgtctatggtgggtccttcagtggttactactggagctggatccgccagcccccagggaaggggctggagtggattggggaaatcaatcatagtggaagcaccaactacaacccgtccctcaagagtcgagtcaccatatcagtagacacgtccaagaaccagttctccctgaagctgagctctgtgaccgccgcggacacggctgtgtattactgtgcgagaagctcgtccggggatgcttttgatatctggggccaagggacaatggtcaccgtctcaagcgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtccacaccttcccggctgtcctacagtcctcaggactctactccctcagcagcgtagtgaccgtgccctccagcagcttgggcacccagacctacatctgcaacgtgaatcacaagcccagcaacaccaaggtggacaagaaagttgagcccaaatcttgt

SEQ ID NO: 346 - DNA, 1431 bp, synthetic construct
  misc_feature 1..1431: note = Synthetic
  source 1..1431: mol_type = other DNA
caggtgcagctacagcagtggggcgcaggactgttgaagccttcggagaccctgtccctcacctgcgctgtctatggtgggtccttcagtggttactactggagctggatccgccagcccccagggaaggggctggagtggattggggaaatcaatcatagtggaagcaccaactacaacccgtccctcaagagtcgagtcaccatatcagtagacacgtccaagaaccagttctccctgaagctgagctctgtgaccgccgcggacacggctgtgtattactgtgcgagaagctcgtccggggatgcttttgatatctggggccaagggacaatggtcaccgtctcaagcggaggaggaggatctggcggaggaggcagtggaggaggagggtcacttaattttatgctgactcagccccactctgtgtcggagtctccggggaagacggtaaccatctcctgcacccgcagcagtggcagcattgccagcaactatgtgcagtggtaccagcagcgcccgggcagttcccccaccacggtcatttttgacgacaatcaaagacccactggtgtccctgatcgcttctctgccgccatcgacacctcctccagttctgcctccctcaccatctctggactgacggctgaggacgaggccgattactattgtcagtcgtctcatagcaccgctgtcgtctttggcggagggaccaagctgaccgtcctaaacagcggcgcgggcaccgcggccgcgactcacacatgcccaccgtgcccagcacctgaagccgcggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatgccgggatgagctgaccaagaaccaggtcagcctgtggtgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 347 - DNA, 657 bp, synthetic construct
  misc_feature 1..657: note = Synthetic
  source 1..657: mol_type = other DNA
gatgttgtgatgactcagtctccactctccctgcccgtcacccctggagagccggcctccatctcctgcaggtctagtcagagcctcctgcatagtaatggatacaactatttggattggtacctgcagaagccagggcagtctccacagctcctgatctatttgggttctagtcgggcctccggggtccctgacaggttcagtggcagtggatcaggcacagattttacactgaaaatcagcagagtggaggctgaggatgttggggtttattactgcatgcaagctctacaaactcctcgcacttttggccaggggaccaagctggagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 348 - DNA, 687 bp, synthetic construct
  misc_feature 1..687: note = Synthetic
  source 1..687: mol_type = other DNA
gaggtgcagctggtgcagtctgggggaggcgtggtccagcctgggaggtccctgagactctcctgtgcagcctctggattcaccttcagtagctatgctatgcactgggtccgccaggctccaggcaaggggctggagtgggtggcagtcatatcatatgatggaagcaataaatactatgcagactccgtgaagggccgattcaccatctccagagacaattccaagaacacgctgtatctgcaaatgaacagcctgagagctgaggacacggctgtgtattactgtgcgagagatctcgggtatagcagcagctggtactactactactacggtatggacgtctggggccaagggaccacggtcaccgtctcaagcgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtccacaccttcccggctgtcctacagtcctcaggactctactccctcagcagcgtagtgaccgtgccctccagcagcttgggcacccagacctacatctgcaacgtgaatcacaagcccagcaacaccaaggtggacaagaaagttgagcccaaatcttgt

SEQ ID NO: 349 - DNA, 1461 bp, synthetic construct
  misc_feature 1..1461: note = Synthetic
  source 1..1461: mol_type = other DNA
gaggtgcagctggtgcagagcggggggggggtggtgcagcctgggaggtcactgagactgagttgtgccgcatccgggtttacatttagctcctatgcaatgcactgggtgaggcaggcccctggcaaggggctggagtgggtggctgtcatcagttacgacggctcaaacaagtactatgcagattctgtgaaagggcggttcacaattagcagagacaactccaaaaatactctgtacctccagatgaatagcctgcgagccgaagacaccgccgtgtactattgcgccagagacctgggatactctagttcatggtactactactactacggcatggacgtgtggggacagggcaccacagtgacagtcagctccggcggaggaggctcaggaggaggagggtccggcggaggaggatctgatgtggtcatgacccagtccccactgtctctgccagtgacacctggcgagccagcaagcatcagctgccggagcagccagtctctgctgcatagtaacgggtataattacctggactggtacttgcagaagcctggccagagtcctcagctgctgatctacctggggtcaagcagggcctccggagtgcccgaccgcttcagtgggtcaggaagcggcactgacttcaccctgaagatcagccgggtggaggctgaagatgtgggcgtctattactgtatgcaggcactgcagacaccacggacttttggacaggggactaaactggaaatcaagaacagcggcgcgggcaccgcggccgcgactcacacatgcccaccgtgcccagcacctgaagccgcggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatgccgggatgagctgaccaagaaccaggtcagcctgtggtgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 350 - DNA, 639 bp, synthetic construct
  misc_feature 1..639: note = Synthetic
  source 1..639: mol_type = other DNA
gacatccagatgacccagtctccatcctccctgtctgcatctgtaggagacagagtcaccatcacttgccaggcgagtcaggacattagcgactatttaaattggtatcagcagaaaccagggaaagcccctcagatcctgatctacgatgcatccaatttggagacaggggtcccatcaagattcagtggaagtgggtctgggacagattttactttcaccatcagcaacctgcagcctgaggatgttgcaacatattactgtcaacagtatgaggatctcccctctttcggcggagggaccaaggtggagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 351 - DNA, 669 bp, synthetic construct
  misc_feature 1..669: note = Synthetic
  source 1..669: mol_type = other DNA
caggtgcagctgcaggagtcgggcccaggactggtgaagccttcggagaccctgtccctcacctgcactgtctctggtgtctccatcagcagtagaagtgaccactggggctgggtccgccagcccccagggaaggggctggagtggattggaagtatctcttatagtgggagcacctactacaacccgtccctcaagagccgagtcaccatatccgtagacacctccaagaaccaactctccctgaagctgagctctgtgaccgccgcagacacggctgtgtattactgtgcgagagagtcgcacccagcagctgcactggttgggtggggccagggcaccctggtcaccgtctcaagcgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtccacaccttcccggctgtcctacagtcctcaggactctactccctcagcagcgtagtgaccgtgccctccagcagcttgggcacccagacctacatctgcaacgtgaatcacaagcccagcaacaccaaggtggacaagaaagttgagcccaaatcttgt

SEQ ID NO: 352 - DNA, 1425 bp, synthetic construct
  misc_feature 1..1425: note = Synthetic
  source 1..1425: mol_type = other DNA
caggtccagctgcaggagagcggccccggactggtgaagcctagcgaaacactgagcctgacttgtactgtgagcggcgtgagcattagctcccggagcgaccactggggatgggtgagacagccccctggcaaggggctggagtggatcggaagtatttcatacagcggctccacttactataacccctctctgaaaagtagggtgactatctcagtggacaccagcaagaatcagctgagtctgaaactgtctagtgtgaccgccgctgatacagcagtctactattgcgcccgcgaatcccatcctgccgccgccctggtgggatggggacaggggacactggtgactgtctcaagcggaggaggaggcagtggaggaggagggtcaggaggcgggggaagcgacattcagatgacacagagcccatcctctctgtctgccagtgtgggcgatcgagtcaccatcacatgtcaggcttcccaggacatttctgattacctgaactggtatcagcagaagccagggaaagctccccagatcctgatctacgacgcatccaatctggagacaggcgtgcccagccggttcagcggaagcggctccgggactgatttcacttttaccatctctaacctccagcctgaggacgtggccacctactattgccagcagtatgaggacctgccatcctttggcgggggaacaaaggtcgagatcaagaacagcggcgcgggcaccgcggccgcgactcacacatgcccaccgtgcccagcacctgaagccgcggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatgccgggatgagctgaccaagaaccaggtcagcctgtggtgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 353 - DNA, 642 bp, synthetic construct
  misc_feature 1..642: note = Synthetic
  source 1..642: mol_type = other DNA
gacatccagttgacccagtctccatcctccctgtctgcatctgtaggagacagagtcaccatcacttgccgggcaagtcagagcattagcagctatttaaattggtatcagcagaaaccagggaaagcccctaaggccctgatctacgatgcatccaatttggaaacaggggtcccatcaaggttcagtggcagtggatctgggacagatttcactctcaccatcatcagtctgcaacctgaagattttgcaacttatttctgtctacaagattacatttacccgtggacgttcggccaagggaccaaggtggaattcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 354 - DNA, 645 bp, synthetic construct
  misc_feature 1..645: note = Synthetic
  source 1..645: mol_type = other DNA
gaggtgcagctggtgcagtctgggggaggcttggtacagcctggggggtccctgagactctcctgtgcagcctctggattcacctttagcagctatgccatgagctgggtccgccaggctccagggaaggggctggagtgggtctcagctattagtggtagtggtggcagcacacactacgcagactccgtgaagggccggttcaccatctccagagacaactccaagaacacgctgtatctgcaaatgaacagcctgagagccgaggacacggccgtatattactgtgcgactccggctttctggggccagggaaccctggtcaccgtctcaagcgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtccacaccttcccggctgtcctacagtcctcaggactctactccctcagcagcgtagtgaccgtgccctccagcagcttgggcacccagacctacatctgcaacgtgaatcacaagcccagcaacaccaaggtggacaagaaagttgagcccaaatcttgt

SEQ ID NO: 355 - DNA, 1404 bp, synthetic construct
  misc_feature 1..1404: note = Synthetic
  source 1..1404: mol_type = other DNA
gaggtgcagctggtgcagagcgggggaggactggtgcagcctggggggtcactgagactgagttgtgccgcaagcgggtttacatttagctcctacgccatgtcttgggtgcgacaggctcccggaaaaggcctggagtgggtcagcgcaatcagtggatcaggcgggtctactcactacgccgacagtgtgaaaggccggttcaccatcagccgggacaacagtaagaatactctgtacctccagatgaacagcctgagagctgaagacaccgccgtgtactattgcgccacccctgctttttgggggcagggaacactggtgactgtctctagtggaggaggaggatcaggcggcggaggcagcggaggaggagggtccgacatccagctgacacagtccccatcaagcctgagcgcttccgtgggcgatagggtcaccatcacatgtcgcgcatctcagagtatttcctcttacctgaactggtatcagcagaagcccggcaaggcacctaaggccctgatctacgacgccagcaatctggagaccggcgtgccttcccggttctcaggcagcgggtccggaacagattttactctgaccatcatcagcctccagccagaggacttcgctacctatttttgcctccaggattacatctacccctggaccttcggccaggggacaaaagtggagttcaagaacagcggcgcgggcaccgcggccgcgactcacacatgcccaccgtgcccagcacctgaagccgcggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatgccgggatgagctgaccaagaaccaggtcagcctgtggtgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 356 - DNA, 651 bp, synthetic construct
  misc_feature 1..651: note = Synthetic
  source 1..651: mol_type = other DNA
cagtctgtgttgacgcagccgccctcagtgtctggggccccagggcagagggtcaccatctcctgcactgggggcagctccaacgtcggggcaggttatgatgtacactggtaccagcagcttccaggaacagtccccaaactcctcatctatgataacaccaatcggccctcaggtgtccctgaccggttctctgcctccaagtctggcacctcagcctctctggtcatcactgggctccaggctgaggatgagggtgactattactgccagtcgtatgacagtagtctgcgtgcttcggtattcggcggagggaccatgttgaccgtcctaagtcagcccaaggctgccccctcggtcactctgttcccgccctcctctgaggagcttcaagccaacaaggccacactggtgtgtctcataagtgacttctacccgggagccgtgacagtggcctggaaggcagatagcagccccgtcaaggcgggagtggagaccaccacaccctccaaacaaagcaacaacaagtacgcggccagcagctatctgagcctgacgcctgagcagtggaagtcccacaaaagctacagctgccaggtcacgcatgaagggagcaccgtggagaagacagtggcccctacagaatgttca

SEQ ID NO: 357 - DNA, 660 bp, synthetic construct
  misc_feature 1..660: note = Synthetic
  source 1..660: mol_type = other DNA
caggtgcagctacagcagtggggcccaggactggtgaagccttcggagaccctgtccctcacctgcactgtctctggtggctccatcagtagttactactggagctggatccgccagcccccagggaaggggctggagtggattggggaaatcaatcatagtggaagcaccaactacaacccgtccctcaagagtcgagtcaccatatcagtagacacgtccaagaaccagttctccctgaagctgagctctgtgaccgccgcggacacggctgtgtattactgtgcgggaggctctaatttggactggttcgacccctggggccagggaaccctggtcaccgtctcaagcgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtccacaccttcccggctgtcctacagtcctcaggactctactccctcagcagcgtagtgaccgtgccctccagcagcttgggcacccagacctacatctgcaacgtgaatcacaagcccagcaacaccaaggtggacaagaaagttgagcccaaatcttgt

SEQ ID NO: 358 - DNA, 1431 bp, synthetic construct
  misc_feature 1..1431: note = Synthetic
  source 1..1431: mol_type = other DNA
caggtccagctgcagcagtgggggccaggactggtgaagccatccgaaactctgtctctgacttgtaccgtgagcggcgggagcatcagctcctactattggagctggatcaggcagccccctgggaagggactggagtggatcggcgaaattaaccacagcgggtccactaactacaatccttccctgaaatctcgcgtgactattagtgtggacacctcaaagaatcagttctccctgaaactgtctagtgtgacagccgctgataccgccgtgtactattgcgccggcgggtctaacctggactggtttgatccctggggacaggggaccctggtgacagtctcaagcggaggaggaggaagcggcggaggaggctccggaggaggagggtctcagagtgtgctgacacagccaccatcagtcagcggggcccccggacagcgagtgaccatctcctgtacaggaggctcctctaatgtgggagccggctacgacgtccattggtatcagcagctgcctggcaccgtgccaaagctgctgatctacgacaacacaaatcggcccagcggggtgcctgatagattctccgcttctaaaagtggcacatcagccagcctggtcatcactggactccaggccgaggacgaaggcgattactattgccagtcttatgatagttcactgagagctagtgtgtttgggggaggcactatgctgaccgtcctgaacagcggcgcgggcaccgcggccgcgactcacacatgcccaccgtgcccagcacctgaagccgcggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatgccgggatgagctgaccaagaaccaggtcagcctgtggtgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 359 - DNA, 651 bp, synthetic construct
  misc_feature 1..651: note = Synthetic
  source 1..651: mol_type = other DNA
aattttatgctgactcagccccactctgtgtcggagtctccggggaagacggtaaccatctcctgcaccggcagcagtggcagcattgccagcagctatgtgcagtggtaccagcagcgcccgggcagtgcccccaccactgtgatctatgcggataaccaaagaccctctggggtccctgatcggttctctggctccgtcgacagctcctccaactctgcctccctcaccatctctggactgaagactgaggacgaggctgactactactgtcagtcttttgacagcagcctctatatgatttttggcggagggaccaagctgaccgtcctaggtcagcccaaggctgccccctcggtcactctgttcccgccctcctctgaggagcttcaagccaacaaggccacactggtgtgtctcataagtgacttctacccgggagccgtgacagtggcctggaaggcagatagcagccccgtcaaggcgggagtggagaccaccacaccctccaaacaaagcaacaacaagtacgcggccagcagctatctgagcctgacgcctgagcagtggaagtcccacagaagctacagctgccaggtcacgcatgaagggagcaccgtggagaagacagtggcccctgcagaatgctct

SEQ ID NO: 360 - DNA, 663 bp, synthetic construct
  misc_feature 1..663: note = Synthetic
  source 1..663: mol_type = other DNA
caggtgcagctacagcagtggggcgcaggactgttgaagccttcggagaccctgtccctcacctgcgctgtctatggtgggtccttcagtggttactactggagctggatccgccagcccccagggaaggggctggagtggattggggaaatcaatcatagtggaagcaccaactacaacccgtccctcaagagtcgagtcaccatatcagtagacacgtccaagaaccagttctccctggagctgagctctgtgaccgccgcggacacggctgtgtattactgtgcgagagcggatcgtcggttcggggagttacgctactggggccagggaaccctggtcaccgtctcaagcgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtccacaccttcccggctgtcctacagtcctcaggactctactccctcagcagcgtagtgaccgtgccctccagcagcttgggcacccagacctacatctgcaacgtgaatcacaagcccagcaacaccaaggtggacaagaaagttgagcccaaatcttgt

SEQ ID NO: 361 - DNA, 1434 bp, synthetic construct
  misc_feature 1..1434: note = Synthetic
  source 1..1434: mol_type = other DNA
caggtccagctgcagcagtggggagccggactgctgaagccaagtgagactctgagcctgacatgcgccgtgtatgggggaagtttttccggctactattggtcttggatcagacagccccctggcaaggggctggagtggatcggcgaaattaaccacagtgggtcaaccaactacaatccctctctgaagagtcgcgtgacaatttcagtggacactagcaaaaatcagttcagcctggagctgagcagcgtgactgccgctgacaccgccgtctactattgcgcacgagccgatcggagatttggcgaactgcggtattggggacagggcacactggtgactgtctctagtggaggaggaggcagtggaggaggagggtcaggaggcgggggatctaacttcatgctgactcagccccatagcgtgtccgagtctcctgggaaaactgtcaccatcagttgtacagggtcaagcggatctattgcctcctcttacgtgcagtggtatcagcagaggccaggctccgctcccaccacagtgatctacgcagacaaccagaggcctagcggagtgccagaccgctttagtggctcagtcgatagttcaagcaatagcgcctccctgaccatctccggcctgaagacagaggacgaagctgattactattgccagagcttcgattcctctctgtatatgatttttggcgggggaaccaaactgacagtgctgaacagcggcgcgggcaccgcggccgcgactcacacatgcccaccgtgcccagcacctgaagccgcggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatgccgggatgagctgaccaagaaccaggtcagcctgtggtgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 362 - DNA, 651 bp, synthetic construct
  misc_feature 1..651: note = Synthetic
  source 1..651: mol_type = other DNA
cagtctgtgttgacgcagccgccctcagtgtctggggccccagggcagagggtcaccatctcctgcactgggagccgctccaacatcggggcaggttatgatgtacactggtatcagcatcttccagggacagcccccaaactcctcatctatgataacagcaatcgaccctcaggtgtctctgaccgattctctggctccaagtctggcacctcagcctccctggccatcactgggctccaggctgaggatgaggctgattattactgccagtcctttgacagcagcctgaggggtgtggtgttcggcggagggaccaggctgaccgtcctaagtcagcccaaggctgccccctcggtcactctgttcccgccctcctctgaggagctccaagccaacaaggccacactagtgtgtctgatcagtgacttctacccgggagctgtgacagtggcctggaaggcagatggcagccccgtcaaggcgggagtggagaccaccacaccctccaaacaaagcaacaacaagtacgcggccagcagctacctgagcctgacgcctgagcagtggaagtcccacaaaagctacagctgccaggtcacgcatgaagggagcaccgtggagaagacagtggcccctacagaatgttca

SEQ ID NO: 363 - DNA, 672 bp, synthetic construct
  misc_feature 1..672: note = Synthetic
  source 1..672: mol_type = other DNA
caggtccagctggtgcagtctgggggaggcctggtcaagcctggggggtccctgagactctcctgtgcagcctctggattcaccttcagtagttatgacttacactgggtccgccaggttccaggcaaggggctggagtgggtgtcacttatatcatatgatggaagtaataaatactatgcagactccgtgaagggccgattcaccatctccagagacaacgccgagaactcactgtatctgcaaatgaacagcctgagagctgaggacacggctgtctattactgtgcgagagagcctataactggaacttctgacctctttgactactggggccagggaaccctggtcaccgtctcaagcgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtccacaccttcccggctgtcctacagtcctcaggactctactccctcagcagcgtagtgaccgtgccctccagcagcttgggcacccagacctacatctgcaacgtgaatcacaagcccagcaacaccaaggtggacaagaaagttgagcccaaatcttgt

SEQ ID NO: 364 - DNA, 1443 bp, synthetic construct
  misc_feature 1..1443: note = Synthetic
  source 1..1443: mol_type = other DNA
caggtgcagctggtgcagagcgggggaggactggtcaagcctggagggtcactgagactgtcatgtgccgcaagcggattcactttcagctcctacgacctgcactgggtgaggcaggtccccggcaaggggctggagtgggtgtctctgatcagttatgacgggagtaacaagtactatgccgattcagtcaaaggacggttcacaatttccagagacaacgctgaaaattctctgtacctccagatgaatagtctgcgcgcagaggatactgccgtgtactattgcgccagagagcctatcaccggcacaagcgacctgtttgattattggggacagggcactctggtgaccgtctctagtggcggaggaggctccggaggaggagggtctggaggaggaggcagccagtctgtgctgacccagccacctagtgtctcaggcgcccctgggcagcgagtgaccatctcctgtacaggcagccggtccaacattggggcaggatacgacgtccactggtatcagcatctgccaggcacagcccccaagctgctgatctacgacaactctaataggccatcaggggtgagcgatcgcttctctggaagtaaatcaggcactagcgcctccctggctattaccggcctccaggctgaggacgaagcagattactattgccagtccttcgattcaagcctgagaggcgtggtctttggcgggggaacaaggctgactgtgctgaacagcggcgcgggcaccgcggccgcgactcacacatgcccaccgtgcccagcacctgaagccgcggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatgccgggatgagctgaccaagaaccaggtcagcctgtggtgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 365 - DNA, 651 bp, synthetic construct
  misc_feature 1..651: note = Synthetic
  source 1..651: mol_type = other DNA
tcctatgagctgactcagccaccctcagcgtctgggacccccgggcagagggtcaccatctcttgttctggaagcagctccaacatcggaagtaatactgtaaactggtaccagcagctcccaggaacggcccccaaactcctcatctatagtaataatcagcggccctcaggggtccctgaccgattctctggctccaagtctggcacctcagcctccctggccatcagtgggctccagtctgaggatgaggctgattattactgtgcagcatgggatgacagcctgaatggtctttgggtgttcggcggagggaccaagctgaccgtcctaggtcagcccaaggctgccccctcggtcactctgttcccaccctcctctgaggagcttcaagccaacaaggccacactggtgtgtctcataagtgacttctacccgggagccgtgacagtggcctggaaggcagatagcagccccgtcaaggcgggagtggagaccaccaaaccctccaaacagagcaacaacaagtacgcggccagcagctacctgagcctgacgcccgagcagtggaagtcccacagaagctacagctgccaggtcacgcatgaagggagcaccgtggagaagacagtggcccctacagaatgttca

SEQ ID NO: 366 - DNA, 660 bp, synthetic construct
  misc_feature 1..660: note = Synthetic
  source 1..660: mol_type = other DNA
caggtgcagctgcaggagtcgggcccaggactggtgaagccttcggggaccctgtccctcacctgcgctgtctctggtggctccatcagcagtagtaactggtggagttgggtccgccagcccccagggaaggggctggagtggattggggaaatctatcatagtgggagcaccaactacaacccgtccctcaagagtcgagtcaccatatcagtagacaagtccaagaaccagttctccctgaagctgagctctgtgaccgccgcggacacggccgtgtattactgtgtcagagaagggggcttacgggaagagcactggggccagggcaccctggtcaccgtctcaagcgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtccacaccttcccggctgtcctacagtcctcaggactctactccctcagcagcgtagtgaccgtgccctccagcagcttgggcacccagacctacatctgcaacgtgaatcacaagcccagcaacaccaaggtggacaagaaagttgagcccaaatcttgt

SEQ ID NO: 367 - DNA, 1452 bp, synthetic construct; identical to SEQ ID NO: 343
  misc_feature 1..1452: note = Synthetic
  source 1..1452: mol_type = other DNA

SEQ ID NO: 368 - DNA, 657 bp, synthetic construct
  misc_feature 1..657: note = Synthetic
  source 1..657: mol_type = other DNA
gatgttgtgatgactcagtctccactctccctgcccgtcacccctggagagccggcctccatctcctgcaggtctagtcagaggctcctgcatagtaatggatacaactatgtggattggtacctgcagaaaccagggcagtctccacagctcctgatctatttgggttctaatcgggcctccggggtccctgacaggttcagtggcagtggatcaggcacagattttacactgaaaatcagcagagtggaggctgaggatgttggggtttattactgcatgcaaggtacacactggccgtggacgttcggccaagggaccaaggtggaaatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 369 - DNA, 660 bp, synthetic construct
  misc_feature 1..660: note = Synthetic
  source 1..660: mol_type = other DNA
caggtgcagctacagcagtggggcgcaggactgttgaagccttcggagaccctgtccctcacctgcgctgtctatggtgggtccttcagtggttactactggagctggatccgccagcccccagggaaggggctggagtggattggggaaatcaatcatagtggaagcaccaactacaacccgtccctcaagagtcgagtcaccatatcagtagacacgtccaagaaccagttctccctgaagctgagctctgtgaccgccgcggacacggctgtgtattactgtgcgagaggcacggatacagctatggctgactactggggccagggaaccctggtcaccgtctcaagcgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtccacaccttcccggctgtcctacagtcctcaggactctactccctcagcagcgtagtgaccgtgccctccagcagcttgggcacccagacctacatctgcaacgtgaatcacaagcccagcaacaccaaggtggacaagaaagttgagcccaaatcttgt

SEQ ID NO: 370 - DNA, 1452 bp, synthetic construct; identical to SEQ ID NO: 343
  misc_feature 1..1452: note = Synthetic
  source 1..1452: mol_type = other DNA

SEQ ID NO: 371 - DNA, 642 bp, synthetic construct
  misc_feature 1..642: note = Synthetic
  source 1..642: mol_type = other DNA
gacatccagatgacccagtctccaccctccctgtccgcatctgtaggagacagagtcaccatcacttgtcaggcgagtcaggacattaacaactatttgaattggtatcaccaaaaaccagggaaggcccctgagctcctgatctacgatgcatctcagttggaaacaggggtcccatcaaggttcagtggaagtggatctgggacagagtttactttcatcatcagcagcctgcagcctgaagataccggtacatattactgtcaacaatatgattggctccccctttcttacggcggagggaccaaggttgagatcaaacgaactgtggctgcaccatctgtcttcatcttcccgccatctgatgagcagttgaaatctggaactgcctctgttgtgtgcctgctgaataacttctatcccagggaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 372 - DNA, 669 bp, synthetic construct
  misc_feature 1..669: note = Synthetic
  source 1..669: mol_type = other DNA
caggtacagctgcagcagtcaggtccaggactggtgaagccctcgcagaccctctcactcacctgtgccatctccggggacagtgtctctggcaacagtgctacttggaactggatcaggcagtccccatcgcgaggccttgagtggctgggaaggacatattacaggtccaagtggaatcatgattatgcagaatctgtgaaaagtcgaataaccatcaacccagacacatccaagaaccagttctccctgcagctgaactctgtgactcccgaggacacggctgtctattactgtgcaagagactccaagtctgcttttgatatctggggccaagggacaatggtcaccgtctcaagcgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtccacaccttcccggctgtcctacagtcctcaggactctactccctcagcagcgtagtgaccgtgccctccagcagcttgggcacccagacctacatctgcaacgtgaatcacaagcccagcaacaccaaggtggacaagaaagttgagcccaaatcttgt

SEQ ID NO: 373 - DNA, 1410 bp, synthetic construct
  misc_feature 1..1410: note = Synthetic
  source 1..1410: mol_type = other DNA
catgtgcagctggtggagactggagggggactggtgcagcctggggggtcactgagactgagttgtgccgcttctgggttcactttcagctcctacgcaatgagctgggtgcggcaggcccccggaaaaggcctggagtgggtctccgccatcagtggatcaggcgggagcacctactatgctgactccgtgaaaggccggttcactattagcagagataactccaagaataccctgtacctccagatgaactccctgagggccgaagacacagctgtctactattgcgctcgcgacctgggcgattattgggggcagggaacactggtgactgtctctagtggaggaggaggatctggaggaggaggcagtggaggaggcgggtcagacatccagctgactcagtctccttcaagcctgagcgcatccatgggggaccgagtcaccatcacatgtcaggccagccaggatattggcaactacctgaattggtatcagctgaagcccggcaaggctcctaagctgctgatctacgacgcatctaatctggagacaggcgtgccaagtagattctctggcagtgggtcaggaactgatttcaccttcaccatcagcagcctccagccagaggacattgccacatactattgcctccagctgtacgattatcccctgacctttggaggcgggacaaaagtggaaatcaagaacagcggcgcgggcaccgcggccgcgactcacacatgcccaccgtgcccagcacctgaagccgcggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatgccgggatgagctgaccaagaaccaggtcagcctgtggtgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 374 - DNA, 651 bp, synthetic construct
  misc_feature 1..651: note = Synthetic
  source 1..651: mol_type = other DNA
aattttatgctgactcagccccactctgtgtcggggtctccggggaagacgataaccatctcctgcacccgcagcagtggcaactttgccagcacctatgtgcagtggtaccaacagcgcccgggcagttcccccgccattgtgatctatgacgatgatcaacgaccctctggtgtccctgaccgcttctctggctccatcgacaggtcctccaactctgcctccctcaccatctctggactggagactgaggacgaggctgactactattgtcagtcttatgatagcagcaatttttgggtgttcggcggagggaccaaactgaccgtcctaggtcagcccaaggctgccccctcggtcactctgttcccaccctcctctgaggagcttcaagccaacaaggccacactggtgtgtctcataagtgacttctacccgggagccgtgacagtggcctggaaggcagatagcagccccgtcaaggcgggagtggagaccaccacaccctccaaacaaagcaacaacaagtacgcggccagcagctacctgagcctgacgcctgagcagtggaagtcccacaaaagctacagctgccaggtcacgcatgaagggagcaccgtggagaagacagtggcccctacagaatgttca

SEQ ID NO: 375 - DNA, 648 bp, synthetic construct
  misc_feature 1..648: note = Synthetic
  source 1..648: mol_type = other DNA
cagctgcagctgcaggagtcgggcccaggactggtgaagccttcggagaccctgtccctcacctgcagtgtctttggtgtctccatcaccagtggtagttggtggagttgggtccgccagtccccagggaaggagctggagtggataggcgaaatctatcataatgggaacaccaactacaacccgtccctcaagagtcgagtcaccatatcggttgacacgtccaagaaccagttctccctgaaactgagctctgtgaccgccgcagacacggctgtctattactgtgtctccggatttgactactggggccagggaaccctggtcaccgtctcaagcgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtccacaccttcccggctgtcctacagtcctcaggactctactccctcagcagcgtagtgaccgtgccctccagcagcttgggcacccagacctacatctgcaacgtgaatcacaagcccagcaacaccaaggtggacaagaaagttgagcccaaatcttgt

SEQ ID NO: 376 - DNA, 1419 bp, synthetic construct
  misc_feature 1..1419: note = Synthetic
  source 1..1419: mol_type = other DNA
cagctgcagctgcaggagagcggccccggactggtgaagcctagcgaaaccctgagcctgacttgttctgtctttggagtgagcatcacttctggaagttggtggagctgggtgagacagtcccccggcaaggagctggaatggatcggggaaatctaccacaacggaaatacaaactataatccttccctgaaatctcgggtgactatcagtgtcgatacctcaaagaaccagttcagcctgaagctgagcagcgtgaccgccgctgatacagccgtgtactattgcgtcagcggctttgactactggggccaggggactctggtgaccgtctctagtggaggaggaggctctggaggaggagggagtggaggaggaggcagcaacttcatgctgacccagcctcattcagtgagcggcagccccggcaagaccatcacaatttcttgtacccgctcaagcgggaattttgctagcacatacgtgcagtggtatcagcagcgacccggctcctctcctgcaatcgtgatctacgacgatgaccagcgaccaagcggcgtccccgatagattctctgggagtatcgacaggagttcaaactcagcaagcctgacaattagcggcctggagactgaagatgaggccgactactattgccagtcctatgacagctccaatttctgggtgtttggcgggggaacaaaactgactgtcctgaacagcggcgcgggcaccgcggccgcgactcacacatgcccaccgtgcccagcacctgaagccgcggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatgccgggatgagctgaccaagaaccaggtcagcctgtggtgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 377 - DNA, 639 bp, synthetic construct
  misc_feature 1..639: note = Synthetic
  source 1..639: mol_type = other DNA
gaaattgtgttgacgcagtctccatcctccctgtctgcatctgtaggagacagagtcaccatcacttgccaggcgagtcaggacattaataattatttaaattggtatcagcagaaaccagggaaagcccctaagctcctgatctacgatgcatccaatttggaaacaggggtcccatcaaagttcagtggaagtggatctgggacagattttactttcaccatcagcagcctgcagcctgaagatattgcaacatattactgtcaacagtatgccaatctcccctcttttggccaggggaccaagctggagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 378 - DNA, 669 bp, synthetic construct
  misc_feature 1..669: note = Synthetic
  source 1..669: mol_type = other DNA
caggtgcagctgcaggagtccggcccaggactggtgaagccttcggagaccctgtctctcacctgcactgtctctggtgtctccatcagcagtagaagtgaccactggggctgggtccgccagcccccagggaaggggctggagtggattggaagtatctcttatagtgggagcacctactacaacccgtccctcaagagccgagtcaccatatccgtagacacctccaagaaccaactctccctgaagctgagctctgtgaccgccgcagacacggctgtgtattactgtgcgagagagtcgcacccagcagctgcactggttgggtggggccagggcaccctggtcaccgtctcaagcgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtccacaccttcccggctgtcctacagtcctcaggactctactccctcagcagcgtagtgaccgtgccctccagcagcttgggcacccagacctacatctgcaacgtgaatcacaagcccagcaacaccaaggtggacaagaaagttgagcccaaatcttgt

SEQ ID NO: 379 - DNA, 1425 bp, synthetic construct
  misc_feature 1..1425: note = Synthetic
  source 1..1425: mol_type = other DNA
caggtgcagctgcaggaaagcggacccggactggtgaagcctagcgagactctgagcctgacttgtaccgtgagcggcgtgagcattagctcccggagcgaccactggggatgggtgagacagccccctggcaaagggctggagtggatcgggagcatttcctactctggaagtacttactataacccctcactgaagagcagggtgactatctccgtggacacctctaaaaatcagctgtctctgaagctgtctagtgtgaccgccgctgatacagcagtctactattgcgcccgcgagtcccatcctgccgccgccctggtgggatggggacaggggacactggtgactgtctcaagcggaggaggaggcagtggaggaggagggtcaggaggcgggggaagcgaaatcgtcctgacacagagtccatcctctctgtcagccagcgtgggcgaccgagtcaccatcacatgtcaggcctcccaggatattaacaattacctgaactggtatcagcagaagccaggcaaagctcccaagctgctgatctacgatgcatccaatctggaaacaggggtgccctctaaattctccggatctggcagtgggactgacttcaccttcaccatcagcagcctccagcctgaggatattgccacctactattgccagcagtatgctaacctgcccagcttcggacagggcacaaaactggaaattaagaacagcggcgcgggcaccgcggccgcgactcacacatgcccaccgtgcccagcacctgaagccgcggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatgccgggatgagctgaccaagaaccaggtcagcctgtggtgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 380 - DNA, 657 bp, synthetic construct
  misc_feature 1..657: note = Synthetic
  source 1..657: mol_type = other DNA
gaaattgtgctgactcagtctccactctccctgcccgtcacccctggagagccggcctccatctcctgcaggtctagtcagagcctcctgcatagtaatggatacaactatttggattggtacctgcagaagccagggcagtctccacagctcctgatctatttgggttctaatcgggcctccggggtccctgacaggttcagtggcagtggatcaggcacagattttacactgagcatcagcagagtggaggctgaggatgttggggtttattactgcatgcaagctctacaaactcctccgacgttcggccaagggaccaaggtggaaatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 381 - DNA, 687 bp, synthetic construct
  misc_feature 1..687: note = Synthetic
  source 1..687: mol_type = other DNA
gaggtccagctggtacagtctggggctgaggtgaagaagcctgggtcctcggtgaaggtctcctgcaaggcttctggttacacctttaccagctatggtatcagctgggtgcgacaggcccctggacaagggcttgagtggatgggatggatcagcgcttacaatggtaacacaaactatgcacagaagctccagggcagagtcaccatgaccacagacacatccacgagcacagcctacatggagctgagcagcctgagatctgaggacacggccgtgtattactgtgcgagagcccctgactacggtgactcctccaactactactactactacatggacgtctggggcaaagggaccacggtcaccgtctcaagcgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtccacaccttcccggctgtcctacagtcctcaggactctactccctcagcagcgtagtgaccgtgccctccagcagcttgggcacccagacctacatctgcaacgtgaatcacaagcccagcaacaccaaggtggacaagaaagttgagcccaaatcttgt

SEQ ID NO: 382 - DNA, 1461 bp, synthetic construct
  misc_feature 1..1461: note = Synthetic
  source 1..1461: mol_type = other DNA
gaagtgcagctggtgcagagcggggcagaggtgaaaaaacctgggtcatccgtcaaagtctcctgtaaggcaagcggctacacatttacttcatacggcatcagctgggtgcgacaggcccctggccaggggctggagtggatgggatggattagcgcatataacggcaatacaaactacgcccagaagctccaggggagagtgactatgaccacagacacaagtacttcaaccgcctatatggagctgagcagcctgaggtccgaagataccgctgtgtactattgcgcccgcgctcctgactacggcgattctagtaactactactactactacatggacgtctggggaaaaggcactaccgtgacagtctcaagcggcggaggaggctccggaggaggagggtctggaggaggaggaagcgagatcgtgctgactcagtctccactgagtctgccagtcacccccggcgaacctgcaagcatttcctgtcggtcctctcagtccctgctgcactctaatgggtataactacctggactggtacttgcagaagccaggacagtctccccagctgctgatctacctgggcagtaaccgagctagcggggtgcctgacagattctctgggagtggatcaggcacagattttactctgagcatcagccgggtggaggctgaagatgtgggcgtctattactgcatgcaggccctccagaccccccctacattcgggcagggaaccaaggtggaaatcaaaaacagcggcgcgggcaccgcggccgcgactcacacatgcccaccgtgcccagcacctgaagccgcggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatgccgggatgagctgaccaagaaccaggtcagcctgtggtgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 383 - DNA, 642 bp, synthetic construct
  misc_feature 1..642: note = Synthetic
  source 1..642: mol_type = other DNA
gacatccagttgacccagtctccatcctccctgtctgcatctgtaggagacagagtcaccgtcacttgccaggcgagccaggacattggccacaatttaaattggtatcagcagagacctgggaaagcccctcagctcctgatctacgatgcatccaatttggaaacaggggtcccatcaaggttcagtggaagtggatctgggacacaatttactttcaccatcagcagtctgcagcctgaagatattgcaacatattactgtcaacaatatgattttctccctcctgacttcggcccagggaccaaagtggagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 384 - DNA, 690 bp, synthetic construct
  misc_feature 1..690: note = Synthetic
  source 1..690: mol_type = other DNA
caggtccagctggtgcagtctggagctgaggtgaagaagcctggggcctcagtgaaggtctcctgcaaggcttctggttacacctttaccagctatggtatcagctgggtgcgacaggcccctggacaagggcttgagtggatgggatggatcagcgcttacaatggtaacacaaactatgcacagaagctccagggcagagtcaccatgaccacagacacatccacgagcacagcctacatggagctgaggagcctgagatctgacgacacggccgtgtattactgtgcgagagatacctccggggactatagcagtggctggtacctaggagttccttttgactactggggccagggcaccctggtcaccgtctcaagcgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtccacaccttcccggctgtcctacagtcctcaggactctactccctcagcagcgtagtgaccgtgccctccagcagcttgggcacccagacctacatctgcaacgtgaatcacaagcccagcaacaccaaggtggacaagaaagttgagcccaaatcttgt

SEQ ID NO: 385 - DNA, 1449 bp, synthetic construct
  misc_feature 1..1449: note = Synthetic
  source 1..1449: mol_type = other DNA
caggtgcagctggtccagagcggagccgaggtgaagaagcccggagcatcagtgaaagtcagttgtaaagcaagcggatacacatttacatcttacggcatcagttgggtgcgacaggcaccaggccaggggctggagtggatgggatggatttctgcatacaacggcaatacaaactatgcccagaagctccaggggagagtcactatgaccacagacactagtacctcaacagcttacatggaactgcggagcctgagatccgacgatactgccgtgtactattgcgctcgggacaccagcggcgattacagctccggctggtatctgggggtccccttcgactattggggacagggcaccctggtgacagtctctagtggcgggggaggctcaggaggaggagggagcggaggaggaggcagcgacatccagctgacccagagcccttcaagcctgagcgcatccgtgggcgacagggtgactgtcacctgccaggcttcccaggacatcgggcacaatctgaactggtatcagcagcgcccaggaaaagctccccagctgctgatctacgacgcatctaatctggagaccggcgtgcccagtcggttttctgggagtggatcaggcacacagttcaccttcaccatcagcagcctccagcctgaggatattgccacttactattgtcagcagtatgacttcctgccccctgattttgggccaggaaccaaggtggagatcaagaacagcggcgcgggcaccgcggccgcgactcacacatgcccaccgtgcccagcacctgaagccgcggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatgccgggatgagctgaccaagaaccaggtcagcctgtggtgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 386 - DNA, 657 bp, synthetic construct
  misc_feature 1..657: note = Synthetic
  source 1..657: mol_type = other DNA
gatgttgtgatgactcagtctccactctccctgcccgtcacccctggagagccggcctccatctcctgcaggtctagtcagagcctcctgcatagtaatggatacaactatttggattggtacctgcagaagccagggcagtctccacagctcctgatctatttgggttctaaccgggactctggggtcccagacagattcagcggcagtgggtcaggcactgatttcacactgaaaatcagcagggtggaggctgaggatgttggggtttattactgcatgcaaggtacacactggccgtggacgttcggccaagggaccaaggtggaaatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 387 - DNA, 669 bp, synthetic construct
  misc_feature 1..669: note = Synthetic
  source 1..669: mol_type = other DNA
caggtgcagctacagcagtggggcgcaggactgttgaagccttcggagaccctgtccctcacctgcgctgtctatggtgggtccttcagtggttactactggagctggatccgccagcccccagggaaggggctggagtggattggggaaatcaatcatagtggaagcaccaactacaacccgtccctcaagagtcgagccaccatatcagtagacacgtccaagaaccagttctccctgaagctgagctctgtgaccgccgcggacacggctgtgtattactgtgcgaccagcccgggaggctattccgggggatacttccagcactggggccagggaaccctggtcaccgtctcaagcgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtccacaccttcccggctgtcctacagtcctcaggactctactccctcagcagcgtagtgaccgtgccctccagcagcttgggcacccagacctacatctgcaacgtgaatcacaagcccagcaacaccaaggtggacaagaaagttgagcccaaatcttgt

SEQ ID NO: 388 - DNA, 1443 bp, synthetic construct
  misc_feature 1..1443: note = Synthetic
  source 1..1443: mol_type = other DNA
caggtccagctgcagcagtggggagccggcctgctgaaaccatctgaaactctgagcctgacttgcgctgtctacggggggtccttcagtggctactattggtcatggatcaggcagccccctgggaagggactggagtggatcggggaaattaaccactccggatctacaaactacaatcccagtctgaaatcacgcgccaccatttctgtggacaccagtaagaatcagttcagcctgaagctgagcagcgtgacagccgctgataccgccgtgtactattgcgcaaccagccctggcggatactccggaggctattttcagcattggggccaggggaccctggtgacagtctctagtgggggaggagggtctggaggaggaggaagtggaggaggaggctccgacgtggtcatgactcagagcccactgtccctgccagtgacccccggcgagcctgctagtatctcatgtcgatcaagccagtcactgctgcacagcaacgggtacaattatctggattggtacttgcagaagccaggccagtctccccagctgctgatctatctgggctccaaccgggactctggggtgcctgatagattcagcggcagcggctctgggactgactttaccctgaaaatttccagagtcgaggcagaagatgtgggagtctactattgcatgcagggcactcattggccctggaccttcggacagggcacaaaggtggagatcaagaacagcggcgcgggcaccgcggccgcgactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtgcaccctgcccccatcccgggatgagctgaccaagaaccaggtcagcctgtcctgcgccgtcaaaggcttctatcccagcgacatcgccgtggagtgggagagcaatgggcagccggagaacaactacaagaccacgcctcccgtgctggactccgacggctccttcttcctcgtgagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 389 - DNA, 639 bp, synthetic construct
  misc_feature 1..639: note = Synthetic
  source 1..639: mol_type = other DNA
tcctatgagctgactcagccaccctcgatgtcagtgtccccaggacagacggccaggatcacctgctctggagatgcattgccaaaacaatttgctttttggtaccagcagaagccaggccaggcccctgtgttggtgatttataaagacactgagaggccctcagggatccctgagcgattctctggctccagctcagggacaacagtcacgttgaccatcactggagtccaggcagaagatgaggctgactattactgtcaatctccagacagcagtggtaccgtcgaagtgttcggcggagggaccaagctgaccgtcctaggtcagcccaaggctgccccctcggtcactctgttcccgccctcctctgaggagcttcaagccaacaaggccacactggtgtgtctcataagtgacttctacccgggagccgtgacagtggcctggaaggcagatagcagccccgtcaaggcgggagtggagaccaccacaccctccaaacaaagcaacaacaagtacgcggccagcagctacctgagcctgacgcctgagcagtggaagtcccacagaagctacagctgccaggtcacgcatgaagggagcaccgtggagaagacagtggcccctgcagaatgt

SEQ ID NO: 390 - DNA, 669 bp, synthetic construct
  misc_feature 1..669: note = Synthetic
  source 1..669: mol_type = other DNA
caggtgcagctgcaggagtcgggcccaggactggtgaagccttcggagaccctgtccctcacctgcactgtctctggtggctccatcagcagtagtagttactactggggctggatccgccagcccccagggaaggggctggagtggattgggagtatctattatagtgggagcacctactacaacccgtccctcaagagtcgagtcaccatatccgtagacacgtccaagaaccagttctccctgaagctgagctctgtgaccgccgcagacacggctgtgtattactgtgcgggcgatattttgactggttatgcccttgactactggggccagggaaccctggtcaccgtctcaagcgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtccacaccttcccggctgtcctacagtcctcaggactctactccctcagcagcgtagtgaccgtgccctccagcagcttgggcacccagacctacatctgcaacgtgaatcacaagcccagcaacaccaaggtggacaagaaagttgagcccaaatcttgt

SEQ ID NO: 391 - DNA, 1431 bp, synthetic construct
  misc_feature 1..1431: note = Synthetic
  source 1..1431: mol_type = other DNA
caggtgcagctgcaggaaagcggacccggactggtgaagccatctgaaacactgagcctgacttgtaccgtgagcggcggaagcatcagctcctctagttactattggggatggatcaggcagccccctggcaaggggctggagtggatcggcagcatctactatagcggctccacatactataaccctagcctgaaatcccgcgtgacaatctctgtggacactagtaagaatcagttctctctgaaactgtcaagcgtgaccgccgctgatacagctgtctactattgcgcaggcgacattctgaccgggtacgccctggattattggggacagggcactctggtgaccgtctcctctggaggaggaggctcaggaggaggagggtccggaggcgggggaagttcatacgaactgacacagccaccctctatgagtgtgtcaccagggcagactgcacgaatcacctgtagcggagacgccctgcccaagcagttcgctttttggtatcagcagaaacctggccaggctccagtgctggtcatctataaggatactgagcggccctctgggattcctgaaagattcagtggcagcagcagcggaaccacagtgactctgaccattacaggcgtgcaggcagaggacgaagccgattactattgccagtcccccgacagttcaggcaccgtggaggtctttggcgggggaacaaaactgactgtgctgaacagcggcgcgggcaccgcggccgcgactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtgcaccctgcccccatcccgggatgagctgaccaagaaccaggtcagcctgtcctgcgccgtcaaaggcttctatcccagcgacatcgccgtggagtgggagagcaatgggcagccggagaacaactacaagaccacgcctcccgtgctggactccgacggctccttcttcctcgtgagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 392 - DNA, 657 bp, synthetic construct
  misc_feature 1..657: note = Synthetic
  source 1..657: mol_type = other DNA
gaaattgtgctgactcagtctccactctccctgcccgttacccctggagagccggcctccatctcctgcaggtctagtcagagcctcctgcatagtaatggatacaactatttgaattggtacctacagaagccagggcagtctccacaactcctgatctatttgggttctgatcgggcctccggggtccctgacaggttcagtggcagtggatcaggcacagattttacactgaaaatcagcagagtggaggctgaggatgttggggtttattactgcatgcaagctctacaaacccccaccactttcggcggagggaccaaggtggagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 393 - DNA, 669 bp, synthetic construct
  misc_feature 1..669: note = Synthetic
  source 1..669: mol_type = other DNA
caggtacagctgcagcagtcaggggctgaggtgaagaagcctgggtcctcggtgaaggtctcctgcaaggcttctggaggcaccttcagcagctatgctatcagctgggtgcgacaggcccctggacaagggcttgagtggatgggaggttttgatcctgaagatggtgaaacaatctacgcacagaagttccagggcagagtcaccatgaccgaggacacatctacagacacagcctacatggagctgagcagcctgagatctgaggacacggccgtgtattactgtgcaacagatctgagaattccgtattactatgataacccctggggccagggcaccctggtcaccgtctcaagcgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtccacaccttcccggctgtcctacagtcctcaggactctactccctcagcagcgtagtgaccgtgccctccagcagcttgggcacccagacctacatctgcaacgtgaatcacaagcccagcaacaccaaggtggacaagaaagttgagcccaaatcttgt

SEQ ID NO: 394 - DNA, 1443 bp, synthetic construct
  misc_feature 1..1443: note = Synthetic
  source 1..1443: mol_type = other DNA
caggtccagctgcagcagagcggagccgaggtcaagaagccagggagtagcgtcaaagtcagttgtaaagcatcaggaggaacattcagctcctatgcaatctcttgggtgcgacaggcccctggacagggcctggagtggatgggaggattcgacccagaggatggagaaaccatctacgcccagaagtttcagggcagagtgactatgaccgaagacacatctactgataccgcttacatggagctgtctagtctgaggagtgaagacactgccgtctactattgcgctaccgacctgcgcatcccatactattacgataatccctgggggcagggaacactggtgactgtctcaagcggaggcgggggatcaggcggaggaggcagcggaggaggagggtccgagatcgtgctgacacagagtccactgtcactgccagtcacccctggcgaaccagccagtatttcatgtcggtcctctcagagcctgctgcactccaacgggtataattacctgaactggtacttgcagaagcctggccagagccctcagctgctgatctacctgggctctgaccgagcaagtggggtgcccgatagattcagcggctccgggtctggaaccgactttaccctgaagatcagccgggtggaggctgaagatgtgggcgtctattactgcatgcaggccctccagacacctaccacattcggaggcgggactaaggtggagatcaagaacagcggcgcgggcaccgcggccgcgactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtgcaccctgcccccatcccgggatgagctgaccaagaaccaggtcagcctgtcctgcgccgtcaaaggcttctatcccagcgacatcgccgtggagtgggagagcaatgggcagccggagaacaactacaagaccacgcctcccgtgctggactccgacggctccttcttcctcgtgagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 395 - DNA, 657 bp, synthetic construct
  misc_feature 1..657: note = Synthetic
  source 1..657: mol_type = other DNA
gaaattgtgatgacgcagtctccactctccctgcccgtcacccctggagagccggcctccatctcctgcaggtctagtcagagcctcctgcatagtaatggatacaactatttggattggtacctgcagaagccagggcagtctccacagctcctgatgtatttggtttctaatcgggcctccggggtccctgagaggttcagtggcagtggatcaggcacagattttacactgaaaatcagcagagtggaggctgaggatgttggggtttattactgcatgcaaactctacaaactcctctcagttttggccaggggaccaagctggagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 396 - DNA, 663 bp, synthetic construct
  misc_feature 1..663: note = Synthetic
  source 1..663: mol_type = other DNA
caggtccagctggtacagtctgggggaggcgtggtccagcctgggaggtccctgagactctcctgtgcagcctctggattcaccttcagtagctatgctatgcactgggtccgccaggctccaggcaaggggctggagtgggtggcagttatatcatatgatggaagcaataaatactacgcagactccgtgaagggccgattcaccatctccagagacaattccaagaacacgctgtatctgcaaatgaacagcctgagagccgaggacacggctgtgtattactgtgcgagaagtctttactacagccactttgactactggggccagggaaccctggtcaccgtctcaagcgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtccacaccttcccggctgtcctacagtcctcaggactctactccctcagcagcgtagtgaccgtgccctccagcagcttgggcacccagacctacatctgcaacgtgaatcacaagcccagcaacaccaaggtggacaagaaagttgagcccaaatcttgt

SEQ ID NO: 397 - DNA, 1437 bp, synthetic construct
  misc_feature 1..1437: note = Synthetic
  source 1..1437: mol_type = other DNA
caggtgcagctggtccagagcggaggaggcgtcgtccagcccggaaggtcactgagactgtcttgtgccgcatcaggattcacttttagctcctacgcaatgcactgggtgaggcaggcccctggcaaggggctggagtgggtggctgtcatcagttatgacggctcaaacaagtactatgcagatagcgtgaaagggcggttcaccattagcagagacaactccaaaaatacactgtacctccagatgaacagcctgcgagccgaagacacagctgtgtactattgcgcccggtctctgtactatagtcactttgattactggggacagggcaccctggtgacagtctctagtggcgggggaggcagtggaggaggagggagcggaggaggaggcagcgagatcgtgatgactcagtccccactgtctctgccagtcacccctggcgaaccagcatccatttcttgtagatcaagccagtcactgctgcatagcaacggatacaattatctggattggtacttgcagaagcctggccagtctcctcagctgctgatgtatctggtgtccaacagggcctctggggtcccagagcgcttcagtgggtcaggaagcggcactgactttaccctgaaaatctctcgcgtggaggctgaagatgtgggcgtctactattgcatgcagacactccagactcccctgagcttcgggcagggaaccaagctggagatcaagaacagcggcgcgggcaccgcggccgcgactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtgcaccctgcccccatcccgggatgagctgaccaagaaccaggtcagcctgtcctgcgccgtcaaaggcttctatcccagcgacatcgccgtggagtgggagagcaatgggcagccggagaacaactacaagaccacgcctcccgtgctggactccgacggctccttcttcctcgtgagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 398 - DNA, 642 bp, synthetic construct
  misc_feature 1..642: note = Synthetic
  source 1..642: mol_type = other DNA
gaaattgtgctgactcagtctccagccaccctgtctttgtctccaggggaacgagccaccctctcctgcagggccagtcagagtgttagttaccacttagcctggtaccaacaaaaacctggccaggctcccaggctcctcatctatgatacatccaacagggcctctggcatccccgccaggttcagtggcagtgggtctgggacagacttcactctcaccatcaacagcctagagcctgaagattttgcagtttattactgtcagcagcgttacgactggcctctcactttcggcggagggaccaaggtggagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 399 - DNA, 678 bp, synthetic construct
  misc_feature 1..678: note = Synthetic
  source 1..678: mol_type = other DNA
gaggtgcagctggtggagactggcccaggactggtgaagccttcggggaccctgtccctcacctgcgctgtctctggtggctccatcagcagtagtaactggtggagttgggtccgccagcccccagggaaggggctggagtggattggggaaatctatcatagtgggagcaccaactacaacccgtccctcaagagtcgagtcaccatatcagtagacaagtccaagaaccagttctccctgaagctgagctctgtgaccgccgcggacacggccgtgtattactgtgcgagagaagggcccctaagcagcagcggaccgggtgcttttgatatctggggccaagggacaatggtcaccgtctcaagcgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtccacaccttcccggctgtcctacagtcctcaggactctactccctcagcagcgtagtgaccgtgccctccagcagcttgggcacccagacctacatctgcaacgtgaatcacaagcccagcaacaccaaggtggacaagaaagttgagcccaaatcttgt

SEQ ID NO: 400 - DNA, 1410 bp, synthetic construct
  misc_feature 1..1410: note = Synthetic
  source 1..1410: mol_type = other DNA
caggtccagctgcaggaatcaggagggggggtcgtccagccagggaggtcactgagactgtcttgcgccgcttcagggttcacttttagcaactacggaatgcactgggtgcggcaggctcccggcaaagggctggagtgggtggcagtcatctcttatgacggcacaaacaagtactatgcagatagtgtcaaggggcggttcaccatcagccgggacaacagtaaaaatacagtgtacctccagatgaacagcctgcgggccgaagatactgctgtctactattgcgccaaggacgggtttgacatctggggacagggcactatggtgaccgtcagctccggcgggggaggctcaggaggaggagggagcggaggaggaggcagcgacattcagatgacccagtcacctagcttcctgtccgcttctgtgggcgatagggtcacaatcacttgtcgcgccagtcagtcaatttctagttggctggcttggtatcagcagaagcccggaaaagcacctaagctgctgatctatgacgcctcccgactggaggatggcgtgccaagcagattctccgggacaggatttggcactgacttcacctttacaatcaccacactccagccagacgatattgccacttactattgccagcagtacgacgatctgccctatacctttgggcagggaactaccgtggatattaagaacagcggcgcgggcaccgcggccgcgactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtgcaccctgcccccatcccgggatgagctgaccaagaaccaggtcagcctgtcctgcgccgtcaaaggcttctatcccagcgacatcgccgtggagtgggagagcaatgggcagccggagaacaactacaagaccacgcctcccgtgctggactccgacggctccttcttcctcgtgagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 401 - DNA, 642 bp, synthetic construct
  misc_feature 1..642: note = Synthetic
  source 1..642: mol_type = other DNA
gacatccagatgacccagtctccttccttcctgtctgcatctgtaggagacagagtcaccatcacttgccgggccagtcagagtattagtagctggttggcctggtatcagcagaaaccagggaaagcccctaaactcctgatctacgatgcatcccgtttggaggacggggtcccatcaagattcagtggaactggatttgggacagattttactttcaccattaccaccctgcagcctgacgatattgcgacatattattgtcagcaatacgatgatctcccgtacacttttggccaggggaccacggtggacatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 402 - DNA, 651 bp, synthetic construct
  misc_feature 1..651: note = Synthetic
  source 1..651: mol_type = other DNA
caggtgcagctgcaggagtccgggggaggcgtggtccagcctgggaggtccctgagactctcctgtgcagcctctggattcaccttcagtaactatggcatgcactgggtccgccaggctccaggcaaagggctggagtgggtggcagttatatcatatgatggaactaataaatactatgcagactccgtgaagggccgattcaccatctccagagacaattccaagaacacggtgtatctgcaaatgaacagcctgagagctgaggacacggctgtgtattactgtgcgaaagatggttttgatatttggggccaagggacaatggtcaccgtctcaagcgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtccacaccttcccggctgtcctacagtcctcaggactctactccctcagcagcgtagtgaccgtgccctccagcagcttgggcacccagacctacatctgcaacgtgaatcacaagcccagcaacaccaaggtggacaagaaagttgagcccaaatcttgt

SEQ ID NO: 403 - DNA, 1437 bp, synthetic construct
  misc_feature 1..1437: note = Synthetic
  source 1..1437: mol_type = other DNA
gaagtgcagctggtggaaactggacctggactggtgaagccaagcgggactctgagcctgacctgtgccgtgagcgggggaagtatcagctcctctaactggtggtcctgggtgcgacagccccctggcaaggggctggagtggatcggcgaaatctaccacagcgggtccacaaactataatcctagcctgaagagccgggtgactatctctgtggacaagagtaaaaatcagttcagcctgaaactgagttcagtgacagccgctgataccgccgtgtactattgcgccagggagggacctctgagcagcagcggaccaggcgcttttgacatctgggggcagggaactatggtgaccgtcagttcaggcggaggaggctccggaggaggagggtctggaggcgggggaagtgagattgtgctgacccagtcccccgccacactgtctctgagtcctggcgaacgggccaccctgtcttgtagagcttcacagagcgtgtcctaccatctggcatggtatcagcagaaaccaggccaggcccccagactgctgatctacgacacctcaaacagggctagcggcattcccgcacgcttctctggcagtgggtcaggaacagattttaccctgacaatcaatagcctggagccagaagacttcgccgtgtactattgccagcagcgctatgattggcccctgacttttggcgggggaaccaaggtcgagatcaagaacagcggcgcgggcaccgcggccgcgactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtgcaccctgcccccatcccgggatgagctgaccaagaaccaggtcagcctgtcctgcgccgtcaaaggcttctatcccagcgacatcgccgtggagtgggagagcaatgggcagccggagaacaactacaagaccacgcctcccgtgctggactccgacggctccttcttcctcgtgagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 404 - DNA, 657 bp, synthetic construct
  misc_feature 1..657: note = Synthetic
  source 1..657: mol_type = other DNA
gatgttgtgatgactcagtctccagtctccctgcccgtcacccttggacagccggcctccatctcctgcaagtctagtcaaagcctcctttactttaatggaaacacctacttgagctggtttcagcagaggccaggccaatctccacggcgcctattttatcaggtttctaaccgggactctggggtcccagacagattcagcggcagtgggtcagacactgatttcactctgaccattagcagggtggaggctgaagatgttggagtttatttctgcatgcaaggaacacagtggcctccgacgttcggccaagggaccaaggtggaaatcaaacgaactgtggctgcaccatctgtcttcatcttcccgccatctgatgagcagctgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 405 - DNA, 669 bp, synthetic construct
  misc_feature 1..669: note = Synthetic
  source 1..669: mol_type = other DNA
gaggtccagctggtgcagtctgggggaggcgtggtccagcctgggaggtccctgagactctcctgtgcagcctctggattcaccttcagtagctatggcatgcactgggtccgccaggctccaggcaaggggctggagtgggtggcagttatatcatatgatggaagtaataaatactatgcagactccgtgaagggccgattcaccatctccagagacaattccaagaacacgctgtatctgcaaatgaacagcctgagagccgaggacacggccgtgtattactgtgcgagagatgtctacggtgactacggggcctttgactactggggccagggaaccctggtcaccgtctcaagcgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtccacaccttcccggctgtcctacagtcctcaggactctactccctcagcagcgtagtgaccgtgccctccagcagcttgggcacccagacctacatctgcaacgtgaatcacaagcccagcaacaccaaggtggacaagaaagttgagcccaaatcttgt

SEQ ID NO: 406 - DNA, 1443 bp, synthetic construct
  misc_feature 1..1443: note = Synthetic
  source 1..1443: mol_type = other DNA
gaggtccagctggtccagagcggcggaggggtcgtccagcccggaagaagcctgagactgtcctgtgcagcaagtgggtttacattcagctcctacggcatgcactgggtgaggcaggcacccggcaaggggctggagtgggtggccgtcatcagttatgacggctcaaacaagtactatgccgatagcgtgaaagggaggttcacaattagccgcgacaactccaaaaatactctgtacctccagatgaacagcctgagagccgaagatacagctgtgtactattgcgctagggacgtctacggagattatggcgcatttgactattggggacagggcactctggtgaccgtctctagtggaggaggaggctcaggaggaggagggagcggcggaggaggcagcgatgtggtcatgacccagtccccagtgtctctgccagtcacactgggacagccagcatccatctcttgtaagtcaagccagtctctgctgtacttcaacggaaatacttatctgtcttggtttcagcagcgccctggccagagtccacggagactgttctaccaggtgtctaaccgagacagtggcgtccctgatcggttcagtgggtcaggaagcgacaccgattttaccctgacaatcagccgagtggaggctgaagacgtgggggtctatttctgcatgcagggaacacagtggccccctacttttggccaggggaccaaggtggagatcaagaacagcggcgcgggcaccgcggccgcgactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtgcaccctgcccccatcccgggatgagctgaccaagaaccaggtcagcctgtcctgcgccgtcaaaggcttctatcccagcgacatcgccgtggagtgggagagcaatgggcagccggagaacaactacaagaccacgcctcccgtgctggactccgacggctccttcttcctcgtgagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 407 - DNA, 657 bp, synthetic construct
  misc_feature 1..657: note = Synthetic
  source 1..657: mol_type = other DNA
gatgttgtgatgactcagtctccactctccctgcccgtcacccctggagagccggcctccatctcctgcaggtctagtcagagcctcctgcatagtaatggatacaactatttggattggtacctgcagaagccagggcagtctccacacctcctgatctacttgggttctaatcgggcctccggggtccctgacaggttcagtggcagtggatcaggcacagattttacactgaaaattagcagagtggaggctgaggatgttggggtttatttctgcatgcaagctctacgaactccgtacacttttggccaggggaccaagctggagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 408 - DNA, 660 bp, synthetic construct
  misc_feature 1..660: note = Synthetic
  source 1..660: mol_type = other DNA
cagctgcagctgcaggagtcggggggaggcgtggtccagcctgggaggtccctgagactctcctgtgcagcctctggattcaccttcagtagctatgctatgcactgggtccgccaggctccaggcaaggggctggagtgggtggcagttatatcatatgatggaggtaataaatactacgcagactccgtgaagggccgattcaccatctccagagacaattccaagaacacgctgtatctgcaaatgaacagcctgagagctgaggacacggctgtgtattactgtgcgaaatcagtggcgcctcccatggacgtctggggcaaagggaccacggtcaccgtctcaagcgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtccacaccttcccggctgtcctacagtcctcaggactctactccctcagcagcgtagtgaccgtgccctccagcagcttgggcacccagacctacatctgcaacgtgaatcacaagcccagcaacaccaaggtggacaagaaagttgagcccaaatcttgt

SEQ ID NO: 409 - DNA, 1434 bp, synthetic construct
  misc_feature 1..1434: note = Synthetic
  source 1..1434: mol_type = other DNA
cagctgcagctgcaggaatccgggggaggcgtcgtccagccaggaaggtcactgagactgagttgtgccgcaagcgggttcactttcagctcctacgctatgcactgggtgagacaggcacccggaaagggcctggagtgggtggcagtcatctcttatgacggcgggaacaagtactatgccgatagtgtgaaaggccggttcaccattagtagagacaactcaaaaaatacactgtacctccagatgaatagcctgcgcgccgaagacacagctgtgtactattgcgcaaagtccgtggccccccctatggatgtctgggggaaaggaaccacagtgactgtctctagtggaggaggaggatcaggcggcggaggcagcggaggaggagggtccgacgtggtcatgactcagtcccctctgtctctgccagtgacccccggcgagcctgcttccatctcttgtaggtcaagccagagcctgctgcactccaacgggtacaattatctggattggtacttgcagaagccaggccagtctccccatctgctgatctatctgggatctaacagggccagtggcgtgcctgaccgcttcagtggctcagggagcggaactgattttaccctgaaaattagccgagtcgaggccgaagatgtgggcgtctacttctgcatgcaggctctgcggacaccatatacttttggccaggggaccaagctggagatcaagaacagcggcgcgggcaccgcggccgcgactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtgcaccctgcccccatcccgggatgagctgaccaagaaccaggtcagcctgtcctgcgccgtcaaaggcttctatcccagcgacatcgccgtggagtgggagagcaatgggcagccggagaacaactacaagaccacgcctcccgtgctggactccgacggctccttcttcctcgtgagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 410 - DNA, 657 bp, synthetic construct
  misc_feature 1..657: note = Synthetic
  source 1..657: mol_type = other DNA
gatgttgtgatgactcagtctccactctccctgcccgtcacccttggacagccggcctccatctcctgcaggtctagtcagagcctcgtccatagtaatggatacaactatttggactggtacctgcagaagccagggcagtctccacagctcctgatctatttgggttctaatcgggcctccggggtccctgacaggttcagtggcagtggatcgggcacagattttacactgaaaatcagcagagtggaggctgaggatgttggggtttattactgcctgcaaggttcacactggccttggacgttcggccaagggaccaaggtggaaatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 411 - DNA, 672 bp, synthetic construct
  misc_feature 1..672: note = Synthetic
  source 1..672: mol_type = other DNA
caggtgcagctacagcagtggggcgcaggactgttgaagccttcggagaccctgtccctcacctgcgctgtctatggtgagtccttcagtggttactactggagctggatccgccagcccccagggaaggggctggagtggattggggaaatcaatcatagtggaagcaccaactacaacccgtccctcaagagtcgagtcaccatatcagtagacacgtccaagaaccagttctccctgaagctgagctctgtgaccgccgcggacacggctgtgtattactgtgcgagaggccccgcgggtagcagctcgtccggctactttgactactggggccagggaaccctggtcaccgtctcaagcgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtccacaccttcccggctgtcctacagtcctcaggactctactccctcagcagcgtagtgaccgtgccctccagcagcttgggcacccagacctacatctgcaacgtgaatcacaagcccagcaacaccaaggtggacaagaaagttgagcccaaatcttgt

SEQ ID NO: 412 - DNA, 1446 bp, synthetic construct
  misc_feature 1..1446: note = Synthetic
  source 1..1446: mol_type = other DNA
caggtccagctgcagcagtggggcgccggactgctgaaaccctctgaaactctgagcctgacttgtgccgtctatggggaatccttctctggctactattggagttggatcaggcagccccctggcaaggggctggagtggatcggagaaattaaccacagcggctccaccaactacaatccatctctgaaaagtcgcgtgaccatttccgtggacacatctaagaatcagttcagcctgaagctgagcagcgtgacagccgctgatactgccgtctactattgcgcacggggccccgccgggtctagttcaagcggatactttgactattggggacagggcaccctggtgacagtctcctctggcggaggaggctccggaggaggagggtctggaggaggaggaagcgatgtggtcatgacacagtcaccactgagcctgccagtgactctgggacagcctgcttctatcagttgtcgaagttcacagagtctggtccactcaaacggatacaattatctggactggtacttgcagaagcctggccagagcccacagctgctgatctatctggggagcaaccgagcttccggagtgcccgacagattctcagggagcggcagcggcactgattttaccctgaaaattagcagagtggaggcagaagatgtgggcgtctactattgcctccaggggtcccattggccttggactttcgggcagggaaccaaggtggagatcaagaacagcggcgcgggcaccgcggccgcgactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtgcaccctgcccccatcccgggatgagctgaccaagaaccaggtcagcctgtcctgcgccgtcaaaggcttctatcccagcgacatcgccgtggagtgggagagcaatgggcagccggagaacaactacaagaccacgcctcccgtgctggactccgacggctccttcttcctcgtgagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 413 - DNA, 639 bp, synthetic construct
  misc_feature 1..639: note = Synthetic
  source 1..639: mol_type = other DNA
gaaacgacactcacgcagtctccagccaccctgtctgtgtctccaggggaaagagccaccctctcctgcagggccagtcagagtgttagcagcaacttagcctggtaccagcagaaacctggccaggctcccaggctcctcatctatggtgcatccagcggggccactggcatcccagacaggttcagtggcagtgggtctgggacagacttcactctcaccatcagcagactggagcctgaagattttgcagtgtattactgtcagctgtatggtagctcactcgctttcggcggagggaccaaggtggagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 414 - DNA, 687 bp, synthetic construct
  misc_feature 1..687: note = Synthetic
  source 1..687: mol_type = other DNA
gaggtccagctggtgcagtctggggctgaggtgaagaagcctgggtcctcggtgaaggtctcctgcaaggcttctggaggcaccttcagcagctatgctatcagctgggtgcgacaggcccctggacaagggcttgagtggatgggatggatcagcgcttacaatggtgacacaagctacgcacagaagttccagggcagagtcaccattaccagggacacatccgcgagcacagcctacatggagctgagcagcctgagatctgaagacacggctgtgtattactgtgcgagagattggggatattgtagtggtggtagctgctacctgaactggttcgacccctggggccagggaaccctggtcaccgtctcaagcgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtccacaccttcccggctgtcctacagtcctcaggactctactccctcagcagcgtagtgaccgtgccctccagcagcttgggcacccagacctacatctgcaacgtgaatcacaagcccagcaacaccaaggtggacaagaaagttgagcccaaatcttgt

SEQ ID NO: 415 - DNA, 1412 bp, synthetic construct
  misc_feature 1..1412: note = Synthetic
  source 1..1412: mol_type = other DNA
aggtccagctgcaggaaagcgggccaggactggtcaaaccctcacagacactgtctctgacttgtaccgtctccgggggctcaatcagctccggcgggtactattggacatggatcagacagcaccctggacagggcctggagtggatcgggttcattagctggtccggaaccacatactataacccaagcctgaagaatagggtgacaatttcagccgacactagcaaaaaccatttttccctgaatctgacctctgtgacagccgctgatactgctgtctactattgcgcacgggggtccggaagactggtgtggggacaggggactctggtgaccgtctctagtggaggaggaggaagtggcggaggaggcagcggaggaggagggtccgagactaccctgacccagtctccagctacactgtctgtgagtcccggcgaaagggcaaccctgagctgtcgcgcttcacagagcgtctcaagcaacctggcatggtatcagcagaagcctggccaggcccctcgactgctgatctatggggcatcctctggagccactggcattcccgaccggttctccggatctggcagtgggaccgattttacactgaccatcagccggctggagcctgaagacttcgctgtgtactattgccagctgtacggcagttcactggcatttggaggcgggacaaaggtcgagatcaagaacagcggcgcgggcaccgcggccgcgactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtgcaccctgcccccatcccgggatgagctgaccaagaaccaggtcagcctgtcctgcgccgtcaaaggcttctatcccagcgacatcgccgtggagtgggagagcaatgggcagccggagaacaactacaagaccacgcctcccgtgctggactccgacggctccttcttcctcgtgagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 416 - DNA, 660 bp, synthetic construct
  misc_feature 1..660: note = Synthetic
  source 1..660: mol_type = other DNA
gatattgtgatgacccacactccactctccctgcccgtcacccctggagagccggcctccatctcctgcaggtctagtcagaccctcttcgatagcgatgatggaaagacctatttggactggtacctgcagaagccagggcagtctccacaactcctgatgtataccacttcctctcgggcctctggagtcccagacaggttcagtggcagtgggtcaggcactgatttcacactgaaaatcagcagggtggaggctgaggatgttggagtttattactgcatgcagcgtttacagtttcccctcaccttcggccaagggacacgactggagttcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 417 - DNA, 669 bp, synthetic construct
  misc_feature 1..669: note = Synthetic
  source 1..669: mol_type = other DNA
gaggtccagctggtgcagtctggggctgaggtgaagaagcctggggcctcagtgaaggtctcctgcaaggcttctggatacaccttcaccggctactatatgcactgggtgcgacaggcccctggacaagggcttgagtggatgggaataatcaaccctagtggtggtagcacaagctacgcacagaagttccagggcagagtcaccatgaccagggacacgtccacgagcacagtctacatggagctgagcagcctgagatctgaggacacggccgtgtattactgtgcgagagccgatacagctatgggtgatgcttttgatatctggggccaagggacaatggtcaccgtctcaagcgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtccacaccttcccggctgtcctacagtcctcaggactctactccctcagcagcgtagtgaccgtgccctccagcagcttgggcacccagacctacatctgcaacgtgaatcacaagcccagcaacaccaaggtggacaagaaagttgagcccaaatcttgt

SEQ ID NO: 418 - DNA, 1446 bp, synthetic construct
  misc_feature 1..1446: note = Synthetic
  source 1..1446: mol_type = other DNA
gaagtccagctggtccagtcaggagccgaggtcaagaagccaggggcaagcgtcaaagtctcatgcaaagcaagtgggtacacatttacaggctactatatgcactgggtgaggcaggctccaggacagggcctggagtggatggggatcattaaccccagcggcgggagtacctcatacgcacagaagttccagggacgggtgactatgaccagagacacaagcacttccaccgtctatatggagctgagcagcctgcgatccgaagacactgccgtgtactattgcgccagagccgataccgcaatgggcgacgcctttgacatctgggggcagggcacaatggtgacagtctctagtggaggaggaggatctggaggaggaggcagtggaggaggcgggtcagacatcgtgatgacacatactccactgtctctgccagtcacccctggcgagccagcctctattagttgtcgctcaagccagaccctgttcgacagtgacgatggaaagacatacctggattggtacttgcagaaacctggccagagccctcagctgctgatgtacaccacatcctctagggcctccggcgtgcctgaccgcttctcaggcagcgggtccggaactgattttaccctgaagatcagccgggtggaggctgaagacgtgggggtctactattgcatgcagagactccagttcccactgacatttggccaggggactcggctggagttcaagaacagcggcgcgggcaccgcggccgcgactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtgcaccctgcccccatcccgggatgagctgaccaagaaccaggtcagcctgtcctgcgccgtcaaaggcttctatcccagcgacatcgccgtggagtgggagagcaatgggcagccggagaacaactacaagaccacgcctcccgtgctggactccgacggctccttcttcctcgtgagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 419 - DNA, 657 bp, synthetic construct
  misc_feature 1..657: note = Synthetic
  source 1..657: mol_type = other DNA
gatgttgtgatgactcagtctccactctccctgcccgtcacccctggagagccggcctccatctcctgcagggcaactcagagcctcctgcatggaaatggacacaactatttggattggtacctgcagaagccagggcagtctccacagctcctgatctatttgggttctaatcgggcctccggggtccctgacaggttcagtggcagtggatcaggcacagattttacactgaaaatcagcagagtggaggctgaggatgttggggtttattactgcatgcaaactctggaaactcctgtcactttcggccctgggaccaaagtggatatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaactctacgcctgcgaagtcacccatcagggcctgagctcgcccgtcacaaagagcttcaacaggggagagtgt

SEQ ID NO: 420 - DNA, 663 bp, synthetic construct
  misc_feature 1..663: note = Synthetic
  source 1..663: mol_type = other DNA
gaggtccagctggtgcagtctgggggaggcgtggtccagcctgggaggtccctgagactctcctgtgcagcctctggattcaccttcagtagctatgctatgcactgggtccgccaggctccaggcaaggggctggagtgggtggcagttatatcatatgatggaagcaataaatactacgcagactccgtgaagggccgattcaccatctccagagacaattccaagaacacgctgtatctgcaaatgaacagcctgagagctgaggacacggctgtgtattactgtgcgaggtctatcggtatcggtgcttttgatatctggggccaagggacaatggtcaccgtctcaagcgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtccacaccttcccggctgtcctacagtcctcaggactctactccctcagcagcgtagtgaccgtgccctccagcagcttgggcacccagacctacatctgcaacgtgaatcacaagcccagcaacaccaaggtggacaagaaagttgagcccaaatcttgt

SEQ ID NO: 421 - DNA, 1437 bp, synthetic construct
  misc_feature 1..1437: note = Synthetic
  source 1..1437: mol_type = other DNA
gaggtccagctggtccagagcggggggggggtcgtgcagcctgggagaagcctgagactgtcctgtgccgcaagcgggtttacttttagctcctacgctatgcactgggtgaggcaggcacccggcaaggggctggagtgggtggcagtcatctcctatgacggctctaacaagtactatgccgatagcgtgaaagggcggttcacaattagtagagacaactcaaagaacactctgtacctccagatgaatagcctgcgagccgaagacactgctgtgtactattgcgcccggtccatcggaattggcgcttttgacatctgggggcagggcacaatggtgacagtctctagtggaggaggaggctctggaggaggagggagtggaggaggaggatcagacgtggtcatgacccagtcacctctgagcctgccagtgacacctggcgagccagcatcaattagctgtagagccacccagtctctgctgcacggcaacgggcataattacctggattggtacttgcagaagcctggccagagtcctcagctgctgatctatctggggagcaacagggcttccggagtgccagaccgcttctccggatctggcagtgggactgattttaccctgaaaatttcccgcgtcgaggcagaagacgtgggagtctactattgcatgcagacactggaaactccagtgaccttcggacccggcacaaaggtggacatcaagaacagcggcgcgggcaccgcggccgcgactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtgcaccctgcccccatcccgggatgagctgaccaagaaccaggtcagcctgtcctgcgccgtcaaaggcttctatcccagcgacatcgccgtggagtgggagagcaatgggcagccggagaacaactacaagaccacgcctcccgtgctggactccgacggctccttcttcctcgtgagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 422 - DNA, 657 bp, synthetic construct
  misc_feature 1..657: note = Synthetic
  source 1..657: mol_type = other DNA
gatgttgtgatgactcagtctccactctccctgcccgtcacccctggagagccggcctccatctcctgcaggtctagtcagagcctcctgcatagtaatggatacaactatttggattggtacctgcagaagccagggcagtctccacagctcctgatctatttgggttctaatcgggcctccggggtccctgacaggttcagtggcagtggatcaggcacagattttacactgaaaatcagcagggtggaggctgaggatgttggggtttattactgcatgcaaggtacacactggccctggacgttcggccaagggaccaaggtggaaatcaaacgaactgtggctgcaccatctgtcttcatcttcccgccatctgatgagcagttgaaatctggaactgcctctgttgtgtgcctgctgaataatttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaactctacgcctgcgaagtcacccatcagggcctgagctcgcccgtcacaaagagcttcaacaggggagagtgt

SEQ ID NO: 423 - DNA, 672 bp, synthetic construct
  misc_feature 1..672: note = Synthetic
  source 1..672: mol_type = other DNA
caggtgcagctacagcagtggggcgcaggactgttgaagccttcggagaccctgtccctcacctgcactatctatggtgggtccttcagtggtttctactggagctggatccgccagcccccagggaagggactggagtggattggggaaatcaatcatagtggaagcaccaactacaacccgtccctcaagagtcgagtcaccatatcagtagacacgtccaagaaccagttctccctgaagctgagctctgtgaccgccgcggacacggctatatattactgtgcgagaggccccgcgggatccacctcgtccggctactttgaccactggggccagggaaccctggtcaccgtctcaagcgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtccacaccttcccggctgtcctacagtcctcaggactctactccctcagcagcgtagtgaccgtgccctccagcagcttgggcacccagacctacatctgcaacgtgaatcacaagcccagcaacaccaaggtggacaagaaagttgagcccaaatcttgt

SEQ ID NO: 424 - DNA, 1446 bp, synthetic construct
  misc_feature 1..1446: note = Synthetic
  source 1..1446: mol_type = other DNA
caggtccagctgcagcagtggggagccggactgctgaaaccctctgagactctgagcctgacttgcacaatctacgggggatcattcagcggcttctactggtcctggatcaggcagccccctggcaaggggctggagtggatcggagaaattaaccacagtggctcaacaaactataatcccagcctgaaatcccgcgtgaccatctcagtggacacaagcaagaatcagttcagcctgaagctgagcagcgtgacagccgctgatactgccatctactattgcgcacggggccctgccgggtccacctctagtgggtactttgaccattggggacagggcaccctggtgacagtctcaagcggaggaggaggctctggaggaggagggagtggaggcgggggcagcgatgtggtcatgactcagtctccactgagtctgccagtgacccccggcgagcctgctagcatctcctgtcgatcctctcagtccctgctgcactctaacggatacaattatctggactggtacttgcagaagccaggccagagcccccagctgctgatctatctggggagtaaccgggcttcaggagtgcctgacagattctctgggagtggatcaggcactgattttaccctgaaaattagcagagtcgaggcagaagatgtgggcgtctactattgcatgcaggggactcattggccctggacctttgggcagggaacaaaggtggagatcaagaacagcggcgcgggcaccgcggccgcgactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtgcaccctgcccccatcccgggatgagctgaccaagaaccaggtcagcctgtcctgcgccgtcaaaggcttctatcccagcgacatcgccgtggagtgggagagcaatgggcagccggagaacaactacaagaccacgcctcccgtgctggactccgacggctccttcttcctcgtgagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 425 - DNA, 657 bp, synthetic construct
  misc_feature 1..657: note = Synthetic
  source 1..657: mol_type = other DNA
gatgttgtgatgactcagtctccactctccctgcccgtcacccctggagagccggcctccatctcctgcaggtctagtcagagcctcctgcatagtaatggatacaactatttggattggtacctgcagaagccagggcagtctccacagctcctgatctatttgggttctaatcgggcctccggggtccctgacaggttcagtggcagtggatcaggcacagattttacactgaaaatcagcagggtggaggctgaggatgttggggtttattactgcctgcaaggtacacattggccgtggacgttcggccaggggaccaaggtggaaatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 426 - DNA, 660 bp, synthetic construct
  misc_feature 1..660: note = Synthetic
  source 1..660: mol_type = other DNA
caggtgcagctacagcagtggggcgcaggactgttgaagccttcggagaccctgtccctcacctgcgctgtctatggtgggtccctcagtggttactactggagctggatccgccagcccccagggaaggggctggagtggattggggaaatcaatcatagtggaagcaccaactacaacccatccctcaagagtcgagtcaccatatcagtagacacgtccaagaaccagttctccctgaagctgagctctgtgaccgccgcggacacggctgtgtattactgtgcgagaggcagcagctcctactacatggacgtctggggcaaagggaccacggtcaccgtctcaagcgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtccacaccttcccggctgtcctacagtcctcaggactctactccctcagcagcgtagtgaccgtgccctccagcagcttgggcacccagacctacatctgcaacgtgaatcacaagcccagcaacaccaaggtggacaagaaagttgagcccaaatcttgt

SEQ ID NO: 427 - DNA, 1434 bp, synthetic construct
  misc_feature 1..1434: note = Synthetic
  source 1..1434: mol_type = other DNA
caggtccagctgcagcagtggggagccggactgctgaaaccaagcgagactctgagcctgacttgtgccgtgtatgggggaagcctgtccggctactattggtcttggatcaggcagccccctggcaaggggctggagtggatcggcgaaattaaccactcagggagcacaaactacaatccctccctgaaatctcgcgtgaccattagcgtggacacatccaagaatcagttcagcctgaagctgagcagcgtgacagccgctgacaccgccgtgtactattgcgccagaggcagcagcagctactatatggatgtgtggggaaagggcaccacagtgaccgtcagctccggaggaggaggcagtggaggaggagggtccggaggcgggggatctgacgtggtcatgactcagagtcctctgtcactgcctgtgacccccggcgagcctgcatccatctcttgtcgatctagtcagtctctgctgcacagtaacggctacaattatctggattggtacttgcagaagccagggcagtccccccagctgctgatctatctgggatcaaaccgggctagcggcgtgcctgacagattcagtgggtcaggaagcggcactgattttaccctgaaaattagcagagtcgaggcagaagatgtgggggtctactattgcctccagggaactcattggccctggacctttgggcagggaacaaaggtggagatcaagaacagcggcgcgggcaccgcggccgcgactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtgcaccctgcccccatcccgggatgagctgaccaagaaccaggtcagcctgtcctgcgccgtcaaaggcttctatcccagcgacatcgccgtggagtgggagagcaatgggcagccggagaacaactacaagaccacgcctcccgtgctggactccgacggctccttcttcctcgtgagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 428 - DNA, 657 bp, synthetic construct
  misc_feature 1..657: note = Synthetic
  source 1..657: mol_type = other DNA
gatgttgtgatgactcagtctccactctccctgcccgtcacccttggtcagccggcctccatctcctgcaggtctagtcagagcctcctgcacagtaatggaaacaactatttggattggtacctgcagaagccagggcagtctccacagctcctgatctatttgggttctaatcgggcctccggggtccctgacaggttcagtggcagtggatcaggcacagattttacactgaaaatcagcagggtggaggctgaggatgttgggatttattactgcatgcaagggacacactggccttggacgttcggccaagggaccaaggtggaaatcgaacgaactgtggctgcaccatctgtcttcatcttcccgccatctgatgagcagttgaaatctggaactgcctctgttgtgtgcctgctgaataacttctatcccagagaggccaaagtacagtggaaggtggatagcgccctccaatcgggtaactcccaggagagtgtcacagagcaggacagcaaggacagcacctacagcctcagcagcaccctgacgctgagcaaagcagactacgagaaacacaaactctacgcctgcgaagtcacccatcagggcctgagctcgcccgtcacaaagagcttcaacaggggagagtgt

SEQ ID NO: 429 - DNA, 657 bp, synthetic construct
  misc_feature 1..657: note = Synthetic
  source 1..657: mol_type = other DNA
caggtgcagctacagcagtggggcgcaggactgttgaagccttcggagaccctgtccctcacctgcgctgtctatggtgggtccttcagtggttactactggagctggatccgccagcccccagggaaggggctggagtggattggggaaatcaatcatagtggaagcaccaactacaacccgtccctcaagagtcgagtcaccatatcagtagacacgtccaagaaccagttctccctgaagctgagctctgtgaccgccgcggacacggctgtgtattactgtgcgagaggcggtagcgcgtacttccagcactggggccagggaaccctggtcaccgtctcaagcgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtccacaccttcccggctgtcctacagtcctcaggactctactccctcagcagcgtagtgaccgtgccctccagcagcttgggcacccagacctacatctgcaacgtgaatcacaagcccagcaacaccaaggtggacaagaaagttgagcccaaatcttgt

SEQ ID NO: 430 - DNA, 1431 bp, synthetic construct
  misc_feature 1..1431: note = Synthetic
  source 1..1431: mol_type = other DNA
caggtccagctgcagcagtggggggccgggctgctgaaaccttccgaaactctgtctctgacttgtgccgtgtatggggggtcctttagtggctactattggtcatggatcaggcagccccctggaaagggcctggagtggatcggagaaattaaccactccggctctacaaactacaatccaagtctgaaatcacgcgtgaccatttctgtggacaccagtaagaatcagttcagcctgaagctgagcagcgtgacagccgctgataccgccgtgtactattgcgcccgaggcgggtctgcttattttcagcattgggggcagggaaccctggtgacagtctctagtggaggaggaggcagcggcggaggaggctctggaggaggagggagtgacgtggtcatgactcagagcccactgtccctgccagtgaccctgggacagccagctagtatctcatgtagatcaagccagtcactgctgcacagcaacggcaacaattacctggattggtacttgcagaagcctggccagagcccacagctgctgatctacctggggtccaatcgggcatctggagtgcccgacagattcagcggctccgggtctggaactgattttaccctgaagatcagccgggtggaggccgaagacgtcggcatctactattgcatgcaggggactcattggccttggaccttcggccaggggacaaaagtggagatcgaaaacagcggcgcgggcaccgcggccgcgactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtgcaccctgcccccatcccgggatgagctgaccaagaaccaggtcagcctgtcctgcgccgtcaaaggcttctatcccagcgacatcgccgtggagtgggagagcaatgggcagccggagaacaactacaagaccacgcctcccgtgctggactccgacggctccttcttcctcgtgagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 431 - DNA, 657 bp, synthetic construct
  misc_feature 1..657: note = Synthetic
  source 1..657: mol_type = other DNA
gaaattgtgctgactcagtctccactctccctgcccgtcacccctggagagccggcctccatctcctgcaggtctagtcagagcctcctgcatagtaatggatacaactatttggattggtacctgcagaagccagggcagtctccacagctcctgatctatttggcttctaatcgggcctccggggtcccagacagattcagcggcagtgggtcaggcactgatttcacactgaaaatcagcagggtggaggctgaggatgttggggtttattactgcatgcaaggtacacactggccgtggacgttcggccaagggaccaaggtggaagtcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccgggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 432 - DNA, 672 bp, synthetic construct
  misc_feature 1..672: note = Synthetic
  source 1..672: mol_type = other DNA
caggtgcagctacagcagtggggcgcaggactgttgaagccttcggagaccctgtccctcacctgcgctgtctatggtgggtccttcagtggttactactggagctggatccgccagcccccagggaaggggctggagtggattggggaaatcaatcatagtggaagcaccaactacaacccgtccctcaagagtcgagtcaccatatcagaagacgcgtccaagaagcagttctccctgacgctgacctctgtgaccgccgcggacacggctgtctattactgtgcgagaggccccgcgggtaccggctcgtccggctactttgactactggggccagggaaccctggtcaccgtctcaagcgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtccacaccttcccggctgtcctacagtcctcaggactctactccctcagcagcgtagtgaccgtgccctccagcagcttgggcacccagacctacatctgcaacgtgaatcacaagcccagcaacaccaaggtggacaagaaagttgagcccaaatcttgt

SEQ ID NO: 433 - DNA, 1446 bp, synthetic construct
  misc_feature 1..1446: note = Synthetic
  source 1..1446: mol_type = other DNA
caggtccagctgcagcagtggggagccggactgctgaagcctagcgaaactctgagcctgacttgtgctgtctacggaggatcatttagtggctactattggtcatggatcaggcagccccctggcaaggggctggagtggatcggagaaattaaccactccggctctacaaactacaatcccagtctgaaatcacgcgtgactatttctgaggacgccagtaagaaacagttctccctgaccctgacatctgtgaccgccgctgatacagctgtctactattgcgcacggggccctgccggaacaggcagctccggatactttgactattgggggcagggaactctggtgaccgtctctagtggcggaggaggcagtggaggaggagggtccggaggaggaggatctgagatcgtgctgactcagagcccactgtccctgccagtcacccccggcgaacctgccagtatttcatgtcgatcaagccagtcactgctgcacagcaacggatacaattatctggactggtacttgcagaagccaggccagagcccccagctgctgatctatctggcttccaatcgggcatctggcgtgcctgacagattcagcggctccgggtctggaacagattttactctgaaaatttccagagtggaggccgaagatgtgggggtctactattgcatgcagggaactcattggccctggaccttcggccaggggacaaaggtggaagtcaaaaacagcggcgcgggcaccgcggccgcgactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtgcaccctgcccccatcccgggatgagctgaccaagaaccaggtcagcctgtcctgcgccgtcaaaggcttctatcccagcgacatcgccgtggagtgggagagcaatgggcagccggagaacaactacaagaccacgcctcccgtgctggactccgacggctccttcttcctcgtgagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 434 - protein, 551 aa, Homo sapiens
MAAPALSWRLPLLILLLPLATSWASAAVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKDTIPWLGHLLVGLSGAFGFIILVYLLINCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 435 - protein, 525 aa, Homo sapiens
AVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKDTIPWLGHLLVGLSGAFGFIILVYLLINCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 436 - protein, 214 aa, Homo sapiens
AVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKDT

SEQ ID NO: 437 - protein, 369 aa, Homo sapiens
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLQNLVIPWAPENLTLHKLSESQLELNWNNRFLNHCLEHLVQYRTDWDHSWTEQSVDYRHKFSLPSVDGQKRYTFRVRSRFNPLCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 438 - protein, 347 aa, Homo sapiens
LNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLQNLVIPWAPENLTLHKLSESQLELNWNNRFLNHCLEHLVQYRTDWDHSWTEQSVDYRHKFSLPSVDGQKRYTFRVRSRFNPLCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 439 - protein, 240 aa, Homo sapiens
LNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLQNLVIPWAPENLTLHKLSESQLELNWNNRFLNHCLEHLVQYRTDWDHSWTEQSVDYRHKFSLPSVDGQKRYTFRVRSRFNPLCGSAQHWSEWSHPIHWGSNTSKENPFLFALEA

SEQ ID NO: 440 - protein, 330 aa, Homo sapiens [sequence omitted]

SEQ ID NO: 441 - protein, 98 aa, synthetic construct
  REGION 1..98: note = Synthetic
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKV

SEQ ID NO: 442 - protein, 12 aa, synthetic construct
  REGION 1..12: note = Synthetic
EPKSCDKTHTCP

SEQ ID NO: 443 - protein, 113 aa, synthetic construct
  REGION 1..113: note = Synthetic
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAK

SEQ ID NO: 444 - protein, 107 aa, synthetic construct
  REGION 1..107: note = Synthetic
GQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 445 - protein, 107 aa, synthetic construct [sequence omitted]

SEQ ID NO: 446 - protein, 220 aa, synthetic construct
  REGION 1..220: note = Synthetic
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 447 - protein, 107 aa, synthetic construct; identical to SEQ ID NO: 260
  REGION 1..107: note = Synthetic

SEQ ID NO: 448 - protein, 107 aa, synthetic construct; identical to SEQ ID NO: 262
  REGION 1..107: note = Synthetic

SEQ ID NO: 449 - protein, 220 aa, synthetic construct
  REGION 1..220: note = Synthetic
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 450 - protein, 220 aa, synthetic construct
  REGION 1..220: note = Synthetic
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 451 - protein, 113 aa, synthetic construct; identical to SEQ ID NO: 259
  REGION 1..113: note = Synthetic

SEQ ID NO: 452 - protein, 220 aa, synthetic construct
  REGION 1..220: note = Synthetic
PCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 453 - protein, 220 aa, synthetic construct
  REGION 1..220: note = Synthetic
PCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 454 - protein, 20 aa, synthetic construct
  REGION 1..20: note = Synthetic
GGGGSGGGGSGGGGSGGGGS

SEQ ID NO: 455 - protein, 5 aa, synthetic construct
  REGION 1..5: note = Synthetic; Linker 6
GGGGS

(19) United States
(12) Patent Application Publication
(10) Pub. No.: US 2024/0052046 A1
(43) Pub. Date: Feb. 15, 2024

(54) IL2RBETA/COMMON GAMMA CHAIN ANTIBODIES

(71) Applicants: Agency for Science, Technology and Research, Singapore (SG); Euchloe Bio Pte. Ltd., Singapore (SG)

(72) Inventors: Peter Brauer, Singapore (SG); John Edward Connolly, Singapore (SG); Richard Hopkins, Singapore (SG); Junyun Lai, Singapore (SG); Jianrong Lionel Low, Singapore (SG); Kar Wai Tan, Singapore (SG); Cheng-I Wang, Singapore (SG); Siok Ping Yeo, Singapore (SG)

(73) Assignees: Agency for Science, Technology and Research, Singapore (SG); Euchloe Bio Pte. Ltd., Singapore (SG)

(21) Appl. No.: 18/344,711

(22) Filed: Jun. 29, 2023

Related U.S. Application Data
(63) Continuation of application No. 16/762,895, filed on May 8, 2020, now Pat. No. 11,739,157.
(60) Provisional application No. 62/735,347, filed on Sep. 24, 2018.
(60) Provisional application No. 62/652,501, filed on Apr. 4, 2018.

(30) Foreign Application Priority Data
Nov. 10, 2017 (SG) 10201709289S

(51) Int. Cl. C07K 16/28; A61P 35/00; C07K 16/46

(52) U.S. Cl. CPC C07K 16/2866; A61P 35/00; C07K 16/468; C07K 2317/565; C07K 2317/75

(57) ABSTRACT

Antigen binding molecules capable of binding to CDI22 and/or common y′ chain (CDI32) are disclosed herein. Also disclosed are compositions comprising such antigen binding molecules, and uses and methods using the same.

### RELATED APPLICATIONS

This application is a continuation of U.S. application Ser. No. 16/762,895, filed May 8, 2020, which is a national stage filing under 35 U.S.C. 371 of International Patent Application Serial No. PCT/EP2018/080765, filed Nov. 9, 2018, which claims priority to Singapore Application No. SG10201709289S, filed Nov. 10, 2017, U.S. Provisional Application No. 62/652,501, filed Apr. 4, 2018, and U.S. Provisional Application No. 62/735,347 filed Sep. 24, 2018, the contents of each of which are incorporated by reference in their entirety.

### REFERENCE TO AN ELECTRONIC SEQUENCE LISTING

The contents of the electronic sequence listing (T088770001US01-SEQ-AZW.xml; Size: 680,792 bytes; and Date of Creation: Jun. 29, 2023) is herein incorporated by reference in its entirety.

### FIELD OF THE INVENTION

The present invention relates to the fields of molecular biology and methods of medical treatment and prophylaxis. In particular, the present invention relates to antigen-binding molecules capable of binding to interleukin 2 receptorβ(IL-2Rβ; CD122) and common y chain (ye; CD132).

### BACKGROUND TO THE INVENTION

IL-2 is an essential cytokine that plays a central role in maintaining T cell homeostasis and mediating proper immune responses. Its high potency as an immune stimulator has led to clinical uses to treat a range of conditions, including cancers and AIDS; it is also widely used as an adjuvant for vaccination to stimulate activation and proliferation of various effector cells. However, the high dose of IL-2 that is required for effective treatment of certain diseases is highly toxic. Major adverse effects of such therapy include vascular leak syndrome (VLS), which results in accumulation of the intravascular fluid in organs such as lung and liver with subsequent pulmonary edema and liver damage. There is no treatment for VLS except withdrawing therapy.

IL-2 exerts its pleiotropic functions by binding to different combinations of receptor components expressed on different cell types: the alpha chain (IL-2Ra, also known as CD25), the beta chain (IL-2R, or CD122), and the common cytokine receptor gamma chain (IL-2Ry, ye, or CD132). Isolated IL-2Ra has been termed the “low affinity” IL-2 receptor (binding affinity Ko˜ 10 nM) and is not involved in signal transduction. A complex of IL-2R and ye binds IL-2 with intermediate affinity (Ko˜ 1 nM), although IL-2RB alone has very low affinity (Ko˜ 100 nM) and ye alone has virtually no detectable binding affinity for IL-2. A complex with all three subunits, IL-2Ra, IL-2Rβ, and ye, binds IL-2 with high affinity (Ko˜10 pM).

Heterodimerization of IL-2RB and ye is necessary and sufficient for effective signal transduction through the interaction of their cytoplasmic domains and subsequent kinase activation of multiple signaling pathways; IL-2Ra plays no role in signal transduction.

High-affinity a-B-yc IL-2Rs are typically found on CD4+ T regulatory cells (Tregs) as well as recently-activated T cells. Intermediate-affinity β-ye IL-2Rs are present at a low level on na″ive CDS+ cells, but are prominent on antigen-experienced (memory) and memory-phenotype (MP) CDS+ T cells as well as natural killer (NK) cells. Both MPCD8+ T cells and NK cells express very high levels of IL-2Rβ and readily respond to IL-2.

Previous studies have indicated that VLS is caused by the release of proinflammatory cytokines from IL-2-activated NK cells. However, a recent study suggested that IL-2-induced pulmonary adema may result from direct binding of IL-2 to lung endothelial cells, which express functional high affinity a-B-yc IL-2Rs. This was evidenced by the observation that interaction of IL-2 with lung endothelial cells was abrogated by blocking anti-IL-2Ra monoclonal antibody (mAb), in IL-2Ra-deficient host mice, or by the use of an IL-2/anti-IL-2 mAb (IL-2/mAb) complex in which the antibody prevents IL-2/IL-2Rx interaction, thus preventing VLS.

### SUMMARY OF THE INVENTION

The present invention relates to antigen-binding molecules, that bind to CD122 (i.e. IL-2Rβ) and/or common γ chain (γc; CD132).

In one aspect, the present invention provides an antigen-binding molecule, optionally isolated, which is capable of binding to CD122 and CD132.

In some embodiments the antigen-binding molecule comprises:
(a) an antigen-binding molecule which is capable of binding to CD122, comprising:
   a heavy chain variable (VH) region incorporating the following CDRs:
      HC-CDR1 having the amino acid sequence of one of SEQ ID NOs: 105, 106, or 108 to 115
      HC-CDR2 having the amino acid sequence of one of SEQ ID NOs: 119 to 127
      HC-CDR3 having the amino acid sequence of one of SEQ ID NOs:133 to 144; and
   a light chain variable (VL) region incorporating the following CDRs:
      LC-CDR1 having the amino acid sequence of one of SEQ ID NOs:151 to 161 LC-CDR2 having the amino acid sequence of one of SEQ ID NOs: 164, or 169 to 176
      LC-CDR3 having the amino acid sequence of one of SEQ ID NOs: 182 to 194;
   or a variant thereof in which one or two or three amino acids in one or more of HC-CDR1, HC-CDR2. HC-CDR3, LC-CDR1, LC-CDR2 or LC-CDR3 are substituted with another amino acid, and
(b) an antigen-binding molecule which is capable of binding to CD132, comprising:
   a VH region incorporating the following CDRs:
      HC-CDR1 having the amino acid sequence of one of SEQ ID NOs:106, 108, 112, or 195 to 201
      HC-CDR2 having the amino acid sequence of one of SEQ ID NOS:119, 120, 124, or 202 to 209
      HC-CDR3 having the amino acid sequence of one of SEQ ID NOs:210 to 225:
   a VL region incorporating the following CDRs.
      LC-CDR1 having the amino acid sequence of one of SEQ ID NOs:151, or 226 to
      LC-CDR2 having the amino acid sequence of one of SEQ ID NOS:174, or 236 to
      LC-CDR3 having the amino acid sequence of one of SEQ ID NOs:189, or 247 to 258;
   or a variant thereof in which one or two or three amino acids in one or more of HC-CDR 1, HC-CDR2, HC-CDR3, LC-CDR1, LC-CDR2 or LC-CDR3 are substituted with another amino acid.

In some embodiments the antigen-binding molecule which is capable of binding to CD122 comprises:
{P1 E7) a VH region incorporating the following CDRs:
   HC-CDR1 having the amino acid sequence of SEQ ID NO: 108
   HC-CDR2 having the amino acid sequence of SEQ ID NO: 120
   HC-CDR3 having the amino acid sequence of SEQ ID NO: 133; and
a VL region incorporating the following CDRs:
   LC-CDR1 having the amino acid sequence of SEQ ID NO: 151
   LC-CDR2 having the amino acid sequence of SEQ ID NO: 169
   LC-CDR3 having the amino acid sequence of SEQ ID NO: 182; or
(P1 B10) a VH region incorporating the following CDRs:
   HC-CDR1 having the amino acid sequence of SEQ ID NO: 109
   HC-CDR2 having the amino acid sequence of SEQ ID NO: 121
   HC-CDR3 having the amino acid sequence of SEQ ID NO: 134; and
a VL region incorporating the following CDRs:
   LC-CDR1 having the amino acid sequence of SEQ ID NO: 152
   LC-CDR2 having the amino acid sequence of SEQ ID NO: 164
   LC-CDR3 having the amino acid sequence of SEQ ID NO: 183; or
{P1 F3) a VH region incorporating the following CDRs:
   HC-CDR1 having the amino acid sequence of SEQ ID NO: 105
   HC-CDR2 having the amino acid sequence of SEQ ID NO: 122
   HC-CDR3 having the amino acid sequence of SEQ ID NO: 135; and
a VL region incorporating the following CDRs:
   LC-CDR1 having the amino acid sequence of SEQ ID NO: 153
   LC-CDR2 having the amino acid sequence of SEQ ID NO: 164
   LC-CDR3 having the amino acid sequence of SEQ ID NO: 184; or
(P1010) a VH region incorporating the following CDRs:
   HC-CDR1 having the amino acid sequence of SEQ ID NO: 110
   HC-CDR2 having the amino acid sequence of SEQ ID NO: 119
   HC-CDR3 having the amino acid sequence of SEQ ID NO: 136; and
a VL region incorporating the following CDRs:
   LC-CDR1 having the amino acid sequence of SEQ ID NO: 154
   LC-CDR2 having the amino acid sequence of SEQ ID NO: 170
   LC-CDR3 having the amino acid sequence of SEQ ID NO: 185; or
{P1 E1) a VH region incorporating the following CDRs:
   HC-CDR1 having the amino acid sequence of SEQ ID NO: 106
   HC-CDR2 having the amino acid sequence of SEQ ID NO: 119
   HC-CDR3 having the amino acid sequence of SEQ ID NO: 137; and
a VL region incorporating the following CD Rs:
   LC-CDR1 having the amino acid sequence of SEQ ID NO: 155
   LC-CDR2 having the amino acid sequence of SEQ ID NO: 171
   LC-CDR3 having the amino acid sequence of SEQ ID NO: 186; or
(P2B11) a VH region incorporating the following CDRs:
   HC-CDR1 having the amino acid sequence of SEQ ID NO: 111
   HC-CDR2 having the amino acid sequence of SEQ ID NO: 123
   HC-CDR3 having the amino acid sequence of SEQ ID NO: 138; and
a VL region incorporating the following CD Rs:
   LC-CDR1 having the amino acid sequence of SEQ ID NO: 156
   LC-CDR2 having the amino acid sequence of SEQ ID NO: 172
   LC-CDR3 having the amino acid sequence of SEQ ID NO: 187; or
(P2C9) a VH region incorporating the following CDRs:
   HC-CDR1 having the amino acid sequence of SEQ ID NO: 112
   HC-CDR2 having the amino acid sequence of SEQ ID NO: 124
   HC-CDR3 having the amino acid sequence of SEQ ID NO: 139; and
a VL region incorporating the following CD Rs:
   LC-CDR1 having the amino acid sequence of SEQ ID NO: 157
   LC-CDR2 having the amino acid sequence of SEQ ID NO: 173
   LC-CDR3 having the amino acid sequence of SEQ ID NO: 188; or
(P2C10) a VH region incorporating the following CDRs:
   HC-CDR1 having the amino acid sequence of SEQ ID NO: 106
   HC-CDR2 having the amino acid sequence of SEQ ID NO: 119
   HC-CDR3 having the amino acid sequence of SEQ ID NO: 140; and
a VL region incorporating the following CD Rs:
   LC-CDR1 having the amino acid sequence of SEQ ID NO: 158
   LC-CDR2 having the amino acid sequence of SEQ ID NO: 174
   LC-CDR3 having the amino acid sequence of SEQ ID NO: 189; or
(P2C11) a VH region incorporating the following CDRs:
   HC-CDR1 having the amino acid sequence of SEQ ID NO: 113
   HC-CDR2 having the amino acid sequence of SEQ ID NO: 125
   HC-CDR3 having the amino acid sequence of SEQ ID NO: 141; and
a VL region incorporating the following CD Rs:
   LC-CDR1 having the amino acid sequence of SEQ ID NO: 159
   LC-CDR2 having the amino acid sequence of SEQ ID NO: 175
   LC-CDR3 having the amino acid sequence of SEQ ID NO: 190; or
(P2E6) a VH region incorporating the following CDRs:
   HC-CDR1 having the amino acid sequence of SEQ ID NO: 114
   HC-CDR2 having the amino acid sequence of SEQ ID NO: 126
   HC-CDR3 having the amino acid sequence of SEQ ID NO: 142; and
a VL region incorporating the following CDRs:
   LC-CDR1 having the amino acid sequence of SEQ ID NO: 160
   LC-CDR2 having the amino acid sequence of SEQ ID NO: 176
   LC-CDR3 having the amino acid sequence of SEQ ID NO: 191; or
(P2E11) a VH region incorporating the following CDRs:
   HC-CDR1 having the amino acid sequence of SEQ ID NO: 109
   HC-CDR2 having the amino acid sequence of SEQ ID NO: 121
   HC-CDR3 having the amino acid sequence of SEQ ID NO: 134; and
a VL region incorporating the following CDRs:
   LC-CDR1 having the amino acid sequence of SEQ ID NO: 159
   LC-CDR2 having the amino acid sequence of SEQ ID NO: 164
   LC-CDR3 having the amino acid sequence of SEQ ID NO: 192; or
(P2F9) a VH region incorporating the following CD Rs:
   HC-CDR1 having the amino acid sequence of SEQ ID NO: 115
   HC-CDR2 having the amino acid sequence of SEQ ID NO: 127
   HC-CDR3 having the amino acid sequence of SEQ ID NO: 143; and
a VL region incorporating the following CDRs:
   LC-CDR1 having the amino acid sequence of SEQ ID NO: 151
   LC-CDR2 having the amino acid sequence of SEQ ID NO: 174
   LC-CDR3 having the amino acid sequence of SEQ ID NO: 193; or
(P2F10) a VH region incorporating the following CDRs:
   HC-CDR1 having the amino acid sequence of SEQ ID NO: 115
   HC-CDR2 having the amino acid sequence of SEQ ID NO: 127
   HC-CDR3 having the amino acid sequence of SEQ ID NO: 144; and
a VL region incorporating the following CDRs:
   LC-CDR1 having the amino acid sequence of SEQ ID NO: 161
   LC-CDR2 having the amino acid sequence of SEQ ID NO: 164
   LC-CDR3 having the amino acid sequence of SEQ ID NO: 194.

In some embodiments the antigen-binding molecule which is capable of binding to CD122 comprises:
a VH region comprising an amino acid sequence having at least 85% sequence identity to one of SEQ ID NOS:22 to 34; and
a VL region comprising an amino acid sequence having at least 85% sequence identity to one of SEQ ID NOS:53 to 65.

In some embodiments the antigen-binding molecule which is capable of binding to CD122 comprises:
(P1 E7) a VH region comprising an amino acid sequence having at least 85% sequence identity to SEQ ID NO: 22; and
a VL region comprising an amino acid sequence having at least 85% sequence identity to SEQ ID NO: 53; or
(P1B10) a VH region comprising an amino acid sequence having at least 85% sequence identity to SEQ ID NO: 23; and
a VL region comprising an amino acid sequence having at least 85% sequence identity to SEQ ID NO: 54; or
(P1F3) a VH region comprising an amino acid sequence having at least 85% sequence identity to SEQ ID NO: 24; and
a VL region comprising an amino acid sequence having at least 85% sequence identity to SEQ ID NO: 55; or
(P1D10) a VH region comprising an amino acid sequence having at least 85% sequence identity to SEQ ID NO: 25; and
a VL region comprising an amino acid sequence having at least 85% sequence identity to SEQ ID NO: 56; or
(P1E1) a VH region comprising an amino acid sequence having at least 85% sequence identity to SEQ ID NO: 26; and
a VL region comprising an amino acid sequence having at least 85% sequence identity to SEQ ID NO: 57; or
(P2B11) a VH region comprising an amino acid sequence having at least 85% sequence identity to SEQ ID NO: 27; and
a VL region comprising an amino acid sequence having at least 85% sequence identity to SEQ ID NO: 58; or
(P2C9) a VH region comprising an amino acid sequence having at least 85% sequence identity to SEQ ID NO: 28; and
a VL region comprising an amino acid sequence having at least 85% sequence identity to SEQ ID NO: 59; or
(P2C10) a VH region comprising an amino acid sequence having at least 85% sequence identity to SEQ ID NO: 29; and
a VL region comprising an amino acid sequence having at least 85% sequence identity to SEQ ID NO: 60; or
(P2C11) a VH region comprising an amino acid sequence having at least 85% sequence identity to SEQ ID NO: 30; and
a VL region comprising an amino acid sequence having at least 85% sequence identity to SEQ ID NO: 61; or
(P2E6) a VH region comprising an amino acid sequence having at least 85% sequence identity to SEQ ID NO: 31; and
a VL region comprising an amino acid sequence having at least 85% sequence identity to SEQ ID NO: 62; or
(P2E11) a VH region comprising an amino acid sequence having at least 85% sequence identity to SEQ ID NO: 32; and
a VL region comprising an amino acid sequence having at least 85% sequence identity to SEQ ID NO: 63; or
(P2F9) a VH region comprising an amino acid sequence having at least 85% sequence identity to SEQ ID NO: 33; and
a VL region comprising an amino acid sequence having at least 85% sequence identity to SEQ ID NO: 64; or
(P2F10) a VH region comprising an amino acid sequence having at least 85% sequence identity to SEQ ID NO: 34; and
a VL region comprising an amino acid sequence having at least 85% sequence identity to SEQ ID NO: 65.

In some embodiments the antigen-binding molecule which is capable of binding to CD132 comprises:
(P1A3) a VH region incorporating the following CDRs:
   HC-CDR1 having the amino acid sequence of SEQ ID NO: 106
   HC-CDR2 having the amino acid sequence of SEQ ID NO: 119
   HC-CDR3 having the amino acid sequence of SEQ ID NO: 210; and
a VL region incorporating the following CD Rs:
   LC-CDR1 having the amino acid sequence of SEQ ID NO: 151
   LC-CDR2 having the amino acid sequence of SEQ ID NO: 236
   LC-CDR3 having the amino acid sequence of SEQ ID NO: 189; or
(P2B9) a VH region incorporating the following CDRs:
   HC-CDR1 having the amino acid sequence of SEQ ID NO: 195
   HC-CDR2 having the amino acid sequence of SEQ ID NO: 202
   HC-CDR3 having the amino acid sequence of SEQ ID NO: 211; and
a VL region incorporating the following CD Rs:
   LC-CDR1 having the amino acid sequence of SEQ ID NO: 226
   LC-CDR2 having the amino acid sequence of SEQ ID NO: 237
   LC-CDR3 having the amino acid sequence of SEQ ID NO: 247; or
(P1A3_B3) a VH region incorporating the following CDRs:
   HC-CDR1 having the amino acid sequence of SEQ ID NO: 106
   HC-CDR2 having the amino acid sequence of SEQ ID NO: 203
   HC-CDR3 having the amino acid sequence of SEQ ID NO: 210; and
a VL region incorporating the following CDRs:
   LC-CDR1 having the amino acid sequence of SEQ ID NO: 151
   LC-CDR2 having the amino acid sequence of SEQ ID NO: 236
   LC-CDR3 having the amino acid sequence of SEQ ID NO: 189; or
(P1A3_B4) a VH region incorporating the following CDRs:
   HC-CDR1 having the amino acid sequence of SEQ ID NO: 106
   HC-CDR2 having the amino acid sequence of SEQ ID NO: 203
   HC-CDR3 having the amino acid sequence of SEQ ID NO: 210; and
a VL region incorporating the following CD Rs:
   LC-CDR1 having the amino acid sequence of SEQ ID NO: 151
   LC-CDR2 having the amino acid sequence of SEQ ID NO: 236
   LC-CDR3 having the amino acid sequence of SEQ ID NO: 189; or
(P1A3_E9) a VH region incorporating the following CDRs:
   HC-CDR1 having the amino acid sequence of SEQ ID NO: 106
   HC-CDR2 having the amino acid sequence of SEQ ID NO: 203
   HC-CDR3 having the amino acid sequence of SEQ ID NO: 210, and
a VL region incorporating the following CDRs:
   LC-CDR1 having the amino acid sequence of SEQ ID NO: 151
   LC-CDR2 having the amino acid sequence of SEQ ID NO: 238
   LC-CDR3 having the amino acid sequence of SEQ ID NO: 189; or
(P1A3_E8) a VH region incorporating the following CDRs:
   HC-CDR1 having the amino acid sequence of SEQ ID NO: 106
   HC-CDR2 having the amino acid sequence of SEQ ID NO: 203
   HC-CDR3 having the amino acid sequence of SEQ ID NO: 210; and
a VL region incorporating the following CDRs:
   LC-CDR1 having the amino acid sequence of SEQ ID NO: 151
   LC-CDR2 having the amino acid sequence of SEQ ID NO: 236
   LC-CDR3 having the amino acid sequence of SEQ ID NO. 189; or
(P1A3_FW2) a VH region incorporating the following CDRs:
   HC-CDR1 having the amino acid sequence of SEQ ID NO: 106
   HC-CDR2 having the amino acid sequence of SEQ ID NO: 119
   HC-CDR3 having the amino acid sequence of SEQ ID NO: 210; and
a VL region incorporating the following CDRs:
   LC-CDR1 having the amino acid sequence of SEQ ID NO: 151
   LC-CDR2 having the amino acid sequence of SEQ ID NO: 236
   LC-CDR3 having the amino acid sequence of SEQ ID NO: 189; or
(P1A10) a VH region incorporating the following CDRs:
   HC-CDR1 having the amino acid sequence of SEQ ID NO: 196
   HC-CDR2 having the amino acid sequence of SEQ ID NO: 204
   HC-CDR3 having the amino acid sequence of SEQ ID NO: 212; and
a VL region incorporating the following CDRs:
   LC-CDR1 having the amino acid sequence of SEQ ID NO: 227
   LC-CDR2 having the amino acid sequence of SEQ ID NO: 238
   LC-CDR3 having the amino acid sequence of SEQ ID NO: 248; or
(P186) a VH region incorporating the following CDRs:
   HC-CDR1 having the amino acid sequence of SEQ ID NO: 108
   HC-CDR2 having the amino acid sequence of SEQ ID NO: 120
   HC-CDR3 having the amino acid sequence of SEQ ID NO: 213; and
a VL region incorporating the following CDRs:
   LC-CDR1 having the amino acid sequence of SEQ ID NO: 151
   LC-CDR2 having the amino acid sequence of SEQ ID NO: 239
   LC-CDR3 having the amino acid sequence of SEQ ID NO: 249; or
(P1C10) a VH region incorporating the following CDRs:
   HC-CDR1 having the amino acid sequence of SEQ ID NO: 112
   HC-CDR2 having the amino acid sequence of SEQ ID NO: 124
   HC-CDR3 having the amino acid sequence of SEQ ID NO: 214, and
a VL region incorporating the following CDRs:
   LC-CDR1 having the amino acid sequence of SEQ ID NO: 228
   LC-CDR2 having the amino acid sequence of SEQ ID NO: 240
   LC-CDR3 having the amino acid sequence of SEQ ID NO: 250; or
(P107) a VH region incorporating the following CDRs:
   HC-CDR 1 having the amino acid sequence of SEQ ID NO: 197
   HC-CDR2 having the amino acid sequence of SEQ ID NO: 206
   HC-CDR3 having the amino acid sequence of SEQ ID NO: 215; and
a VL region incorporating the following CDRs:
   LC-CDR1 having the amino acid sequence of SEQ ID NO 229
   LC-CDR2 having the amino acid sequence of SEQ ID NO: 241
   LC-CDR3 having the amino acid sequence of SEQ ID NO: 251; or
(P1 E8) a VH region incorporating the following CDRs:
   HC-CDR 1 having the amino acid sequence of SEQ ID NO: 198
   HC-CDR2 having the amino acid sequence of SEQ ID NO: 120
   HC-CDR3 having the amino acid sequence of SEQ ID NO: 216; and
a VL region incorporating the following CDRs:
   LC-CDR1 having the amino acid sequence of SEQ ID NO: 230
   LC-CDR2 having the amino acid sequence of SEQ ID NO: 242
   LC-CDR3 having the amino acid sequence of SEQ ID NO: 252; or
(P2B2) a VH region incorporating the following CDRS:
   HC-CDR1 having the amino acid sequence of SEQ ID NO: 108
   HC-CDR2 having the amino acid sequence of SEQ ID NO: 207
   HC-CDR3 having the amino acid sequence of SEQ ID NO: 217; and
a VL region incorporating the following CDRs:
   LC-CDR1 having the amino acid sequence of SEQ ID NO: 151
   LC-CDR2 having the amino acid sequence of SEQ ID NO: 174
   LC-CDR3 having the amino acid sequence of SEQ ID NO: 253; or
(P2B7) a VH region incorporating the following CDRs:
   HC-CDR 1 having the amino acid sequence of SEQ ID NO: 106
   HC-CDR2 having the amino acid sequence of SEQ ID NO: 119
   HC-CDR3 having the amino acid sequence of SEQ ID NO: 218; and
a VL region incorporating the following CDRs:
   LC-CDR1 having the amino acid sequence of SEQ ID NO: 231
   LC-CDR2 having the amino acid sequence of SEQ ID NO: 174
   LC-CDR3 having the amino acid sequence of SEQ ID NO: 254; or
(P2D11) a VH region incorporating the following CDRs:
   HC-CDR 1 having the amino acid sequence of SEQ ID NO: 199
   HC-CDR2 having the amino acid sequence of SEQ ID NO: 208
   HC-CDR3 having the amino acid sequence of SEQ ID NO: 219; and
a VL region incorporating the following CDRS:
   LC-CDR1 having the amino acid sequence of SEQ ID NO: 232
   LC-CDR2 having the amino acid sequence of SEQ ID NO: 243
   LC-CDR3 having the amino acid sequence of SEQ ID NO: 255; or
(P2F10) a VH region incorporating the following CDRs:
   HC-CDR1 having the amino acid sequence of SEQ ID NO: 200
   HC-CDR2 having the amino acid sequence of SEQ ID NO: 209
   HC-CDR3 having the amino acid sequence of SEQ ID NO: 220; and
a VL region incorporating the following CDRs:
   LC-CDR1 having the amino acid sequence of SEQ ID NO: 233
   LC-CDR2 having the amino acid sequence of SEQ ID NO: 244
   LC-CDR3 having the amino acid sequence of SEQ ID NO: 256; or
(P2H4) a VH region incorporating the following CD Rs:
   HC-CDR1 having the amino acid sequence of SEQ ID NO: 108
   HC-CDR2 having the amino acid sequence of SEQ ID NO: 120
   HC-CDR3 having the amino acid sequence of SEQ ID NO: 221; and
a VL region incorporating the following CDRs:
   LC-CDR1 having the amino acid sequence of SEQ ID NO: 234
   LC-CDR2 having the amino acid sequence of SEQ ID NO: 174
   LC-CDR3 having the amino acid sequence of SEQ ID NO: 257; or
(P2D3) a VH region incorporating the following CDRs:
   HC-CDR1 having the amino acid sequence of SEQ ID NO: 201
   HC-CDR2 having the amino acid sequence of SEQ ID NO: 119
   HC-CDR3 having the amino acid sequence of SEQ ID NO: 222; and
a VL region incorporating the following CDRs:
   LC-CDR1 having the amino acid sequence of SEQ ID NO: 151
   LC-CDR2 having the amino acid sequence of SEQ ID NO: 174
   LC-CDR3 having the amino acid sequence of SEQ ID NO: 189; or
(P1G4) a VH region incorporating the following CDRs:
   HC-CDR1 having the amino acid sequence of SEQ ID NO: 106
   HC-CDR2 having the amino acid sequence of SEQ ID NO: 119
   HC-CDR3 having the amino acid sequence of SEQ ID NO: 223; and
a VL region incorporating the following CDRs:
   LC-CDR1 having the amino acid sequence of SEQ ID NO: 151
   LC-CDR2 having the amino acid sequence of SEQ ID NO: 174
   LC-CDR3 having the amino acid sequence of SEQ ID NO: 258; or
(P1 B12) a VH region incorporating the following CDRs:
   HC-CDR1 having the amino acid sequence of SEQ ID NO: 106
   HC-CDR2 having the amino acid sequence of SEQ ID NO: 119
   HC-CDR3 having the amino acid sequence of SEQ ID NO: 224; and
a VL region incorporating the following CDRs:
   LC-CDR1 having the amino acid sequence of SEQ ID NO: 235
   LC-CDR2 having the amino acid sequence of SEQ ID NO: 174
   LC-CDR3 having the amino acid sequence of SEQ ID NO: 189; or
(P1C7) a VH region incorporating the following CDRs:
   HC-CDR1 having the amino acid sequence of SEQ ID NO: 106
   HC-CDR2 having the amino acid sequence of SEQ ID NO: 119
   HC-CDR3 having the amino acid sequence of SEQ ID NO: 225; and
a VL region incorporating the following CDRs:
   LC-CDR1 having the amino acid sequence of SEQ ID NO: 151
   LC-CDR2 having the amino acid sequence of SEQ ID NO: 245
   LC-CDR3 having the amino acid sequence of SEQ ID NO: 189.

In some embodiments the antigen-binding molecule which is capable of binding to CD132 comprises:
a VH region comprising an amino acid sequence having at least 85% sequence identity to one of SEQ ID NOS:66 to 84; and
a VL region comprising an amino acid sequence having at least 85% sequence identity to one of SEQ ID NOS:85 to 102.

In some embodiments the antigen-binding molecule which is capable of binding to CD132 comprises:
(P1A3) a VH region comprising an amino acid sequence having at least 85% sequence identity to SEQ ID NO: 66; and
a VL region comprising an amino acid sequence having at least 85% sequence identity to SEQ ID NO: 85; or
(P2B9) a VH region comprising an amino acid sequence having at least 85% sequence identity to SEQ ID NO: 67; and
a VL region comprising an amino acid sequence having at least 85% sequence identity to SEQ ID NO: 86; or
(P1A3_B3) a VH region comprising an amino acid sequence having at least 85% sequence identity to SEQ ID NO: 68; and
a VL region comprising an amino acid sequence having at least 85% sequence identity to SEQ ID NO: 85; or
(P1A3_B4) a VH region comprising an amino acid sequence having at least 85% sequence identity to SEQ ID NO: 68; and
a VL region comprising an amino acid sequence having at least 85% sequence identity to SEQ ID NO: 87; or
(P1A3_E9) a VH region comprising an amino acid sequence having at least 85% sequence identity to SEQ ID NO: 68; and
a VL region comprising an amino acid sequence having at least 85% sequence identity to SEQ ID NO: 85; or
(P1A3_E8) a VH region comprising an amino acid sequence having at least 85% sequence identity to SEQ ID NO: 69; and
a VL region comprising an amino acid sequence having at least 85% sequence identity to SEQ ID NO: 85; or
(P1A3_FW2) a VH region comprising an amino acid sequence having at least 85% sequence identity to SEQ ID NO: 70; and a VL region comprising an amino acid sequence having at least 85% sequence identity to SEQ ID NO: 88; or
(P1A10) a VH region comprising an amino acid sequence having at least 85% sequence identity to SEQ ID NO: 71; and
a VL region comprising an amino acid sequence having at least 85% sequence identity to SEQ ID NO: 89; or
(P1 B6) a VH region comprising an amino acid sequence having at least 85% sequence identity to SEQ ID NO: 72; and
a VL region comprising an amino acid sequence having at least 85% sequence identity to SEQ ID NO: 90; or
(P1C10) a VH region comprising an amino acid sequence having at least 85% sequence identity to SEQ ID NO: 73; and
a VL region comprising an amino acid sequence having at least 85% sequence identity to SEQ ID NO: 91; or
(P107) a VH region comprising an amino acid sequence having at least 85% sequence identity to SEQ ID NO: 74; and
a VL region comprising an amino acid sequence having at least 85% sequence identity to SEQ ID NO: 92; or
(P1 EB) a VH region comprising an amino acid sequence having at least 85% sequence identity to SEQ ID NO: 75; and
a VL region comprising an amino acid sequence having at least 85% sequence identity to SEQ ID NO: 93; or
(P2B2) a VH region comprising an amino acid sequence having at least 85% sequence identity to SEQ ID NO: 76; and
a VL region comprising an amino acid sequence having at least 85% sequence identity to SEQ ID NO: 94; or (P2B7) a VH region comprising an amino acid sequence having at least 85% sequence identity to SEQ ID NO: 77; and
a VL region comprising an amino acid sequence having at least 85% sequence identity to SEQ ID NO: 95; or
(P2011) a VH region comprising an amino acid sequence having at least 85% sequence identity to SEQ ID NO: 78; and
a VL region comprising an amino acid sequence having at least 85% sequence identity to SEQ ID NO: 96; or
(P2F10) a VH region comprising an amino acid sequence having at least 85% sequence identity to SEQ ID NO: 79; and
a VL region comprising an amino acid sequence having at least 85% sequence identity to SEQ ID NO: 97; or
(P2H4) a VH region comprising an amino acid sequence having at least 85% sequence identity to SEQ ID NO: 80; and
a VL region comprising an amino acid sequence having at least 85% sequence identity to SEQ ID NO: 98; or
(P2D3) a VH region comprising an amino acid sequence having at least 85% sequence identity to SEQ ID NO: 81; and
a VL region comprising an amino acid sequence having at least 85% sequence identity to SEQ ID NO: 99; or
(P1G4) a VH region comprising an amino acid sequence having at least 85% sequence identity to SEQ ID NO: 82; and
a VL region comprising an amino acid sequence having at least 85% sequence identity to SEQ ID NO: 100; or
(P1 B12) a VH region comprising an amino acid sequence having at least 85% sequence identity to SEQ ID NO: 83; and
a VL region comprising an amino acid sequence having at least 85% sequence identity to SEQ ID NO: 101; or
(P1C7) a VH region comprising an amino acid sequence having at least 85% sequence identity to SEQ ID NO: 84; and
a VL region comprising an amino acid sequence having at least 85% sequence identity to SEQ ID NO: 102.

In some embodiments the antigen-binding molecule comprises:
(a) an antigen-binding molecule which is capable of binding to CD122, comprising:
   a heavy chain variable (VH) region incorporating the following CDRs:
      HC-CDR1 having the amino acid sequence of one of SEQ ID NOs:103 to 115
      HC-CDR2 having the amino acid sequence of one of SEQ ID NOs:116 to 127
      HC-CDR3 having the amino acid sequence of one of SEQ ID NOS:128 to 144; and
   a light chain variable (VL) region incorporating the following CDRs:
      LC-CDR1 having the amino acid sequence of one of SEQ ID NOs:145 to 161
      LC-CDR2 having the amino acid sequence of one of SEQ ID NOs:162 to 176
      LC-CDR3 having the amino acid sequence of one of SEQ ID NOs:177 to 194;
   or a variant thereof in which one or MP or three amino acids in one or more of HC-CDR1, HC-CDR2, HC-CDR3, LC-CDR1, LC-CDR2 or LC-CDR3 are substituted with another amino acid.
(b) an antigen-binding molecule which is capable of binding to CD132, comprising:
   a VH region incorporating the following CDRs:
      HC-CDR1 having the amino acid sequence of one of SEQ ID NOs:106, 108, 112, or 196 to 201
      HC-CDR2 having the amino acid sequence of one of SEQ ID NOs:119, 120, 124, or 204 to 209
      HC-CDR3 having the amino acid sequence of one of SEQ ID NOs:212 to 225; and
   a VL region incorporating the following CDRs:
      LC-CDR1 having the amino acid sequence of one of SEQ ID NOs:151, or 227 to
      LC-CDR2 having the amino acid sequence of one of SEQ ID NOs:174, or 238 to
      LC-CDR3 having the amino acid sequence of one of SEQ ID NOs:189, or 248 to 258;
   or a variant thereof in which one or two or three amino acids in one or more of HC-CDR1, HC-CDR2, HC-CDR3, LC-CDR1, LC-CDR2 or LC-CDR3 are substituted with another amino acid.

In some embodiments the antigen-binding molecule which is capable of binding to CD122 comprises:
(P2C4) a VH region incorporating the following CDRs:
   HC-CDR1 having the amino acid sequence of SEQ ID NO: 103
   HC-CDR2 having the amino acid sequence of SEQ ID NO: 116
   HC-CDR3 having the amino acid sequence of SEQ ID NO: 128; and
a VL region incorporating the following CDRs:
   LC-CDR1 having the amino acid sequence of SEQ ID NO: 145
   LC-CDR2 having the amino acid sequence of SEQ ID NO: 162
   LC-CDR3 having the amino acid sequence of SEQ ID NO: 177; or
(P2C4_A4) a VH region incorporating the following CDRs:
   HC-CDR1 having the amino acid sequence of SEQ ID NO: 103
   HC-CDR2 having the amino acid sequence of SEQ ID NO: 116
   HC-CDR3 having the amino acid sequence of SEQ ID NO: 128; and
a VL region incorporating the following CDRs:
   LC-CDR1 having the amino acid sequence of SEQ ID NO: 149
   LC-CDR2 having the amino acid sequence of SEQ ID NO: 162
   LC-CDR3 having the amino acid sequence of SEQ ID NO: 177; or
(P2C4_B1) a VH region incorporating the following CDRs:
   HC-CDR1 having the amino acid sequence of SEQ ID NO: 103
   HC-CDR2 having the amino acid sequence of SEQ ID NO: 116
   HC-CDR3 having the amino acid sequence of SEQ ID NO: 128; and
a VL region incorporating the following CD Rs:
   LC-CDR1 having the amino acid sequence of SEQ ID NO: 145
   LC-CDR2 having the amino acid sequence of SEQ ID NO: 166
   LC-CDR3 having the amino acid sequence of SEQ ID NO: 177; or
(P2C4_B5) a VH region incorporating the following CDRs:
   HC-CDR1 having the amino acid sequence of SEQ ID NO: 103
   HC-CDR2 having the amino acid sequence of SEQ ID NO: 116
   HC-CDR3 having the amino acid sequence of SEQ ID NO: 128; and
a VL region incorporating the following CDRs:
   LC-CDR1 having the amino acid sequence of SEQ ID NO: 145
   LC-CDR2 having the amino acid sequence of SEQ ID NO: 162
   LC-CDR3 having the amino acid sequence of SEQ ID NO: 181; or
(P2C4_C4) a VH region incorporating the following CDRs:
   HC-CDR1 having the amino acid sequence of SEQ ID NO: 103
   HC-CDR2 having the amino acid sequence of SEQ ID NO: 116
   HC-CDR3 having the amino acid sequence of SEQ ID NO: 128; and
a VL region incorporating the following CDRs:
   LC-CDR1 having the amino acid sequence of SEQ ID NO: 145
   LC-CDR2 having the amino acid sequence of SEQ ID NO: 166
   LC-CDR3 having the amino acid sequence of SEQ ID NO: 181; or
(P2C4_C7) a VH region incorporating the following CDRs:
   HC-CDR1 having the amino acid sequence of SEQ ID NO: 103
   HC-CDR2 having the amino acid sequence of SEQ ID NO: 116
   HC-CDR3 having the amino acid sequence of SEQ ID NO: 128; and
a VL region incorporating the following CDRs:
   LC-CDR1 having the amino acid sequence of SEQ ID NO: 145
   LC-CDR2 having the amino acid sequence of SEQ ID NO: 162
   LC-CDR3 having the amino acid sequence of SEQ ID NO: 181; or
(P2C4_D10) a VH region incorporating the following CDRs:
   HC-CDR1 having the amino acid sequence of SEQ ID NO: 103
   HC-CDR2 having the amino acid sequence of SEQ ID NO: 116
   HC-CDR3 having the amino acid sequence of SEQ ID NO: 128; and
a VL region incorporating the following CDRs:
   LC-CDR1 having the amino acid sequence of SEQ ID NO: 145
   LC-CDR2 having the amino acid sequence of SEQ ID NO: 162
   LC-CDR3 having the amino acid sequence of SEQ ID NO: 181; or
(P2C4_E6) a VH region incorporating the following CDRs:
   HC-CDR1 having the amino acid sequence of SEQ ID NO: 103
   HC-CDR2 having the amino acid sequence of SEQ ID NO: 116
   HC-CDR3 having the amino acid sequence of SEQ ID NO: 128; and
a VL region incorporating the following CDRs:
   LC-CDR1 having the amino acid sequence of SEQ ID NO: 149
   LC-CDR2 having the amino acid sequence of SEQ ID NO: 162
   LC-CDR3 having the amino acid sequence of SEQ ID NO: 177; or
(P2C4_E7) a VH region incorporating the following CDRs:
   HC-CDR1 having the amino acid sequence of SEQ ID NO: 103
   HC-CDR2 having the amino acid sequence of SEQ ID NO: 116
   HC-CDR3 having the amino acid sequence of SEQ ID NO: 128; and
a VL region incorporating the following CDRs:
   LC-CDR1 having the amino acid sequence of SEQ ID NO: 145
   LC-CDR2 having the amino acid sequence of SEQ ID NO: 162
   LC-CDR3 having the amino acid sequence of SEQ ID NO: 181; or
(P2C4_F8) a VH region incorporating the following CDRs:
   HC-CDR1 having the amino acid sequence of SEQ ID NO: 103
   HC-CDR2 having the amino acid sequence of SEQ ID NO: 116
   HC-CDR3 having the amino acid sequence of SEQ ID NO: 128; and
a VL region incorporating the following CDRs:
   LC-CDR1 having the amino acid sequence of SEQ ID NO: 145
   LC-CDR2 having the amino acid sequence of SEQ ID NO: 162
   LC-CDR3 having the amino acid sequence of SEQ ID NO: 181; or
(P2H7) a VH region incorporating the following CDRs:
   HC-CDR1 having the amino acid sequence of SEQ ID NO: 104
   HC-CDR2 having the amino acid sequence of SEQ ID NO: 117
   HC-CDR3 having the amino acid sequence of SEQ ID NO: 129; and
a VL region incorporating the following CDRs:
   LC-CDR1 having the amino acid sequence of SEQ ID NO: 146
   LC-CDR2 having the amino acid sequence of SEQ ID NO: 163
   LC-CDR3 having the amino acid sequence of SEQ ID NO: 178; or
(P2D12) a VH region incorporating the following CDRs:
   HC-CDR1 having the amino acid sequence of SEQ ID NO: 105
   HC-CDR2 having the amino acid sequence of SEQ ID NO: 118
   HC-CDR3 having the amino acid sequence of SEQ ID NO: 130; and
a VL region incorporating the following CDRs:
   LC-CDR1 having the amino acid sequence of SEQ ID NO: 147
   LC-CDR2 having the amino acid sequence of SEQ ID NO: 164
   LC-CDR3 having the amino acid sequence of SEQ ID NO: 179; or
(P1G11) a VH region incorporating the following CDRs:
   HC-CDR1 having the amino acid sequence of SEQ ID NO: 106
   HC-CDR2 having the amino acid sequence of SEQ ID NO: 119
   HC-CDR3 having the amino acid sequence of SEQ ID NO: 131; and
a VL region incorporating the following CDRs:
   LC-CDR1 having the amino acid sequence of SEQ ID NO: 148
   LC-CDR2 having the amino acid sequence of SEQ ID NO: 165
   LC-CDR3 having the amino acid sequence of SEQ ID NO: 180; or
(P2C4_A9) a VH region incorporating the following CDRs:
   HC-CDR1 having the amino acid sequence of SEQ ID NO: 103
   HC-CDR2 having the amino acid sequence of SEQ ID NO: 116
   HC-CDR3 having the amino acid sequence of SEQ ID NO: 132; and
a VL region incorporating the following CDRs:
   LC-CDR1 having the amino acid sequence of SEQ ID NO: 145
   LC-CDR2 having the amino acid sequence of SEQ ID NO: 162
   LC-CDR3 having the amino acid sequence of SEQ ID NO: 177; or
(P2C4_B6) a VH region incorporating the following CDRs:
   HC-CDR1 having the amino acid sequence of SEQ ID NO: 107
   HC-CDR2 having the amino acid sequence of SEQ ID NO: 116
   HC-CDR3 having the amino acid sequence of SEQ ID NO: 128; and
a VL region incorporating the following CDRs:
   LC-CDR1 having the amino acid sequence of SEQ ID NO: 145
   LC-CDR2 having the amino acid sequence of SEQ ID NO: 162
   LC-CDR3 having the amino acid sequence of SEQ ID NO: 181; or
(P2C4_E9) a VH region incorporating the following CDRs:
   HC-CDR1 having the amino acid sequence of SEQ ID NO: 107
   HC-CDR2 having the amino acid sequence of SEQ ID NO: 116
   HC-CDR3 having the amino acid sequence of SEQ ID NO: 128; and
a VL region incorporating the following CD Rs:
   LC-CDR1 having the amino acid sequence of SEQ ID NO: 145
   LC-CDR2 having the amino acid sequence of SEQ ID NO: 168
   LC-CDR3 having the amino acid sequence of SEQ ID NO: 181; or
(P2C4_B8) a VH region incorporating the following CDRs:
   HC-CDR1 having the amino acid sequence of SEQ ID NO: 103
   HC-CDR2 having the amino acid sequence of SEQ ID NO: 116
   HC-CDR3 having the amino acid sequence of SEQ ID NO: 128; and
a VL region incorporating the following CD Rs:
   LC-CDR1 having the amino acid sequence of SEQ ID NO: 145
   LC-CDR2 having the amino acid sequence of SEQ ID NO: 162
   LC-CDR3 having the amino acid sequence of SEQ ID NO: 181; or
(P2C4_B12) a VH region incorporating the following CDRs:
   HC-CDR1 having the amino acid sequence of SEQ ID NO: 103
   HC-CDR2 having the amino acid sequence of SEQ ID NO: 116
   HC-CDR3 having the amino acid sequence of SEQ ID NO: 128; and
a VL region incorporating the following CD Rs:
   LC-CDR1 having the amino acid sequence of SEQ ID NO: 150
   LC-CDR2 having the amino acid sequence of SEQ ID NO: 167
   LC-CDR3 having the amino acid sequence of SEQ ID NO: 177; or
(P2C4_C1) a VH region incorporating the following CDRs:
   HC-CDR1 having the amino acid sequence of SEQ ID NO: 103
   HC-CDR2 having the amino acid sequence of SEQ ID NO: 116
   HC-CDR3 having the amino acid sequence of SEQ ID NO: 128; and
a VL region incorporating the following CD Rs:
   LC-CDR1 having the amino acid sequence of SEQ ID NO: 149
   LC-CDR2 having the amino acid sequence of SEQ ID NO: 162
   LC-CDR3 having the amino acid sequence of SEQ ID NO: 177; or
(P2C4_C12) a VH region incorporating the following CDRs:
   HC-CDR1 having the amino acid sequence of SEQ ID NO: 103
   HC-CDR2 having the amino acid sequence of SEQ ID NO: 116
   HC-CDR3 having the amino acid sequence of SEQ ID NO: 128; and
a VL region incorporating the following CD Rs:
   LC-CDR1 having the amino acid sequence of SEQ ID NO: 145
   LC-CDR2 having the amino acid sequence of SEQ ID NO: 162
   LC-CDR3 having the amino acid sequence of SEQ ID NO: 181; or
(P2C4_E2) a VH region incorporating the following CDRs:
   HC-CDR1 having the amino acid sequence of SEQ ID NO: 103
   HC-CDR2 having the amino acid sequence of SEQ ID NO: 116
   HC-CDR3 having the amino acid sequence of SEQ ID NO: 128; and
a VL region incorporating the following CD Rs:
   LC-CDR1 having the amino acid sequence of SEQ ID NO: 145
   LC-CDR2 having the amino acid sequence of SEQ ID NO: 162
   LC-CDR3 having the amino acid sequence of SEQ ID NO: 181; or
(P2C4_E3) a VH region incorporating the following CDRs:
   HC-CDR1 having the amino acid sequence of SEQ ID NO: 107
   HC-CDR2 having the amino acid sequence of SEQ ID NO: 116
   HC-CDR3 having the amino acid sequence of SEQ ID NO: 128; and
a VL region incorporating the following CD Rs:
   LC-CDR1 having the amino acid sequence of SEQ ID NO: 145
   LC-CDR2 having the amino acid sequence of SEQ ID NO: 162
   LC-CDR3 having the amino acid sequence of SEQ ID NO: 181; or
(P2C4_E8) a VH region incorporating the following CDRs:
   HC-CDR1 having the amino acid sequence of SEQ ID NO: 103
   HC-CDR2 having the amino acid sequence of SEQ ID NO: 116
   HC-CDR3 having the amino acid sequence of SEQ ID NO: 128; and
a VL region incorporating the following CD Rs:
   LC-CDR1 having the amino acid sequence of SEQ ID NO: 145
   LC-CDR2 having the amino acid sequence of SEQ ID NO: 162
   LC-CDR3 having the amino acid sequence of SEQ ID NO: 181; or
(P2C4_F11) a VH region incorporating the following CDRs:
   HC-CDR1 having the amino acid sequence of SEQ ID NO: 103
   HC-CDR2 having the amino acid sequence of SEQ ID NO: 116
   HC-CDR3 having the amino acid sequence of SEQ ID NO: 128; and
a VL region incorporating the following CD Rs:
   LC-CDR1 having the amino acid sequence of SEQ ID NO: 145
   LC-CDR2 having the amino acid sequence of SEQ ID NO: 162
   LC-CDR3 having the amino acid sequence of SEQ ID NO: 181; or
(P2C4_G2) a VH region incorporating the following CDRs:
   HC-CDR1 having the amino acid sequence of SEQ ID NO: 103
   HC-CDR2 having the amino acid sequence of SEQ ID NO: 116
   HC-CDR3 having the amino acid sequence of SEQ ID NO: 128; and
a VL region incorporating the following CD Rs:
   LC-CDR1 having the amino acid sequence of SEQ ID NO: 145
   LC-CDR2 having the amino acid sequence of SEQ ID NO: 162
   LC-CDR3 having the amino acid sequence of SEQ ID NO: 181; or
(P2C4_G11) a VH region incorporating the following CDRs:
   HC-CDR1 having the amino acid sequence of SEQ ID NO: 103
   HC-CDR2 having the amino acid sequence of SEQ ID NO: 116
   HC-CDR3 having the amino acid sequence of SEQ ID NO: 128; and
a VL region incorporating the following CDRs:
   LC-CDR1 having the amino acid sequence of SEQ ID NO: 145
   LC-CDR2 having the amino acid sequence of SEQ ID NO: 162
   LC-CDR3 having the amino acid sequence of SEQ ID NO: 181; or
(P2C4_H1) a VH region incorporating the following CDRs:
   HC-CDR1 having the amino acid sequence of SEQ ID NO: 103
   HC-CDR2 having the amino acid sequence of SEQ ID NO: 116
   HC-CDR3 having the amino acid sequence of SEQ ID NO: 128; and
a VL region incorporating the following CDRs:
   LC-CDR1 having the amino acid sequence of SEQ ID NO: 145
   LC-CDR2 having the amino acid sequence of SEQ ID NO: 162
   LC-CDR3 having the amino acid sequence of SEQ ID NO: 181; or
(P2C4_H2) a VH region incorporating the following CDRs:
   HC-CDR1 having the amino acid sequence of SEQ ID NO: 103
   HC-CDR2 having the amino acid sequence of SEQ ID NO: 116
   HC-CDR3 having the amino acid sequence of SEQ ID NO: 128; and
a VL region incorporating the following CDRs:
   LC-CDR1 having the amino acid sequence of SEQ ID NO: 145
   LC-CDR2 having the amino acid sequence of SEQ ID NO: 162
   LC-CDR3 having the amino acid sequence of SEQ ID NO: 181; or
(P2C4_H3) a VH region incorporating the following CDRs:
   HC-CDR1 having the amino acid sequence of SEQ ID NO: 103
   HC-CDR2 having the amino acid sequence of SEQ ID NO: 116
   HC-CDR3 having the amino acid sequence of SEQ ID NO: 128; and
a VL region incorporating the following CDRs:
   LC-CDR1 having the amino acid sequence of SEQ ID NO: 145
   LC-CDR2 having the amino acid sequence of SEQ ID NO: 162
   LC-CDR3 having the amino acid sequence of SEQ ID NO: 181; or
(P2C4_C1D10) a VH region incorporating the following CDRs:
   HC-CDR1 having the amino acid sequence of SEQ ID NO: 103
   HC-CDR2 having the amino acid sequence of SEQ ID NO: 116
   HC-CDR3 having the amino acid sequence of SEQ ID NO: 128; and
a VL region incorporating the following CDRs:
   LC-CDR1 having the amino acid sequence of SEQ ID NO: 149
   LC-CDR2 having the amino acid sequence of SEQ ID NO: 162
   LC-CDR3 having the amino acid sequence of SEQ ID NO: 181; or
(P2C4_FW2) a VH region incorporating the following CDRs:
   HC-CDR1 having the amino acid sequence of SEQ ID NO: 103
   HC-CDR2 having the amino acid sequence of SEQ ID NO: 116
   HC-CDR3 having the amino acid sequence of SEQ ID NO: 128; and
a VL region incorporating the following CDRs:
   LC-CDR 1 having the amino acid sequence of SEQ ID NO: 145
   LC-CDR2 having the amino acid sequence of SEQ ID NO: 162
   LC-CDR3 having the amino acid sequence of SEQ ID NO: 177; or
(P1E7) a VH region incorporating the following CDRs:
   HC-CDR1 having the amino acid sequence of SEQ ID NO: 108
   HC-CDR2 having the amino acid sequence of SEQ ID NO: 120
   HC-CDR3 having the amino acid sequence of SEQ ID NO: 133; and
a VL region incorporating the following CDRs:
   LC-CDR 1 having the amino acid sequence of SEQ ID NO: 151
   LC-CDR2 having the amino acid sequence of SEQ ID NO: 169
   LC-CDR3 having the amino acid sequence of SEQ ID NO: 182; or
(P1 B10) a VH region incorporating the following CDRs:
   HC-CDR1 having the amino acid sequence of SEQ ID NO: 109
   HC-CDR2 having the amino acid sequence of SEQ ID NO: 121
   HC-CDR3 having the amino acid sequence of SEQ ID NO: 134; and
a VL region incorporating the following CDRs:
   LC-CDR 1 having the amino acid sequence of SEQ ID NO: 152
   LC-CDR2 having the amino acid sequence of SEQ ID NO: 164
   LC-CDR3 having the amino acid sequence of SEQ ID NO: 183; or
(P1 F3) a VH region incorporating the following CDRs:
   HC-CDR1 having the amino acid sequence of SEQ ID NO: 105
   HC-CDR2 having the amino acid sequence of SEQ ID NO: 122
   HC-CDR3 having the amino acid sequence of SEQ ID NO: 135; and
a VL region incorporating the following CDRs:
   LC-CDR 1 having the amino acid sequence of SEQ ID NO: 153
   LC-CDR2 having the amino acid sequence of SEQ ID NO: 164
   LC-CDR3 having the amino acid sequence of SEQ ID NO: 184; or
(P1D10) a VH region incorporating the following CDRs:
   HC-CDR1 having the amino acid sequence of SEQ ID NO: 110
   HC-CDR2 having the amino acid sequence of SEQ ID NO: 119
   HC-CDR3 having the amino acid sequence of SEQ ID NO: 136; and
a VL region incorporating the following CDRs:
   LC-CDR 1 having the amino acid sequence of SEQ ID NO: 154
   LC-CDR2 having the amino acid sequence of SEQ ID NO: 170
   LC-CDR3 having the amino acid sequence of SEQ ID NO: 185; or
(P1 E1) a VH region incorporating the following CDRs:
   HC-CDR1 having the amino acid sequence of SEQ ID NO: 106
   HC-CDR2 having the amino acid sequence of SEQ ID NO: 119
   HC-CDR3 having the amino acid sequence of SEQ ID NO: 137; and
a VL region incorporating the following CDRs:
   LC-CDR1 having the amino acid sequence of SEQ ID NO: 155
   LC-CDR2 having the amino acid sequence of SEQ ID NO: 171
   LC-CDR3 having the amino acid sequence of SEQ ID NO: 186; or
(P2B11) a VH region incorporating the following CDRs:
   HC-CDR1 having the amino acid sequence of SEQ ID NO: 111
   HC-CDR2 having the amino acid sequence of SEQ ID NO: 123
   HC-CDR3 having the amino acid sequence of SEQ ID NO: 138; and
a VL region incorporating the following CD Rs:
   LC-CDR1 having the amino acid sequence of SEQ ID NO: 156
   LC-CDR2 having the amino acid sequence of SEQ ID NO: 172
   LC-CDR3 having the amino acid sequence of SEQ ID NO: 187; or
(P2C9) a VH region incorporating the following CDRs:
   HC-CDR1 having the amino acid sequence of SEQ ID NO: 112
   HC-CDR2 having the amino acid sequence of SEQ ID NO: 124
   HC-CDR3 having the amino acid sequence of SEQ ID NO: 139; and
a VL region incorporating the following CD Rs:
   LC-CDR1 having the amino acid sequence of SEQ ID NO: 157
   LC-CDR2 having the amino acid sequence of SEQ ID NO: 173
   LC-CDR3 having the amino acid sequence of SEQ ID NO: 188; or
(P2C10) a VH region incorporating the following CDRs:
   HC-CDR1 having the amino acid sequence of SEQ ID NO: 106
   HC-CDR2 having the amino acid sequence of SEQ ID NO: 119
   HC-CDR3 having the amino acid sequence of SEQ ID NO: 140; and
a VL region incorporating the following CD Rs:
   LC-CDR1 having the amino acid sequence of SEQ ID NO: 158
   LC-CDR2 having the amino acid sequence of SEQ ID NO: 174
   LC-CDR3 having the amino acid sequence of SEQ ID NO: 189; or
(P2C11) a VH region incorporating the following CDRs:
   HC-CDR1 having the amino acid sequence of SEQ ID NO: 113
   HC-CDR2 having the amino acid sequence of SEQ ID NO: 125
   HC-CDR3 having the amino acid sequence of SEQ ID NO: 141; and
a VL region incorporating the following CD Rs:
   LC-CDR1 having the amino acid sequence of SEQ ID NO: 159
   LC-CDR2 having the amino acid sequence of SEQ ID NO: 175
   LC-CDR3 having the amino acid sequence of SEQ ID NO: 190; or
(P2E6) a VH region incorporating the following CDRs:
   HC-CDR1 having the amino acid sequence of SEQ ID NO: 114
   HC-CDR2 having the amino acid sequence of SEQ ID NO: 126
   HC-CDR3 having the amino acid sequence of SEQ ID NO: 142; and
a VL region incorporating the following CD Rs:
   LC-CDR1 having the amino acid sequence of SEQ ID NO: 160
   LC-CDR2 having the amino acid sequence of SEQ ID NO: 176
   LC-CDR3 having the amino acid sequence of SEQ ID NO: 191; or
(P2E11) a VH region incorporating the following CDRs:
   HC-CDR1 having the amino acid sequence of SEQ ID NO: 109
   HC-CDR2 having the amino acid sequence of SEQ ID NO: 121
   HC-CDR3 having the amino acid sequence of SEQ ID NO: 134; and
a VL region incorporating the following CDRs:
   LC-CDR1 having the amino acid sequence of SEQ ID NO: 159
   LC-CDR2 having the amino acid sequence of SEQ ID NO: 164
   LC-CDR3 having the amino acid sequence of SEQ ID NO: 192; or
(P2F9) a VH region incorporating the following CDRs:
   HC-CDR1 having the amino acid sequence of SEQ ID NO: 115
   HC-CDR2 having the amino acid sequence of SEQ ID NO: 127
   HC-CDR3 having the amino acid sequence of SEQ ID NO: 143; and
a VL region incorporating the following CDRs:
   LC-CDR1 having the amino acid sequence of SEQ ID NO: 151
   LC-CDR2 having the amino acid sequence of SEQ ID NO: 174
   LC-CDR3 having the amino acid sequence of SEQ ID NO: 193; or
(P2F10) a VH region incorporating the following CDRs:
   HC-CDR1 having the amino acid sequence of SEQ ID NO: 115
   HC-CDR2 having the amino acid sequence of SEQ ID NO: 127
   HC-CDR3 having the amino acid sequence of SEQ ID NO: 144; and
a VL region incorporating the following CDRs:
   LC-CDR1 having the amino acid sequence of SEQ ID NO: 161
   LC-CDR2 having the amino acid sequence of SEQ ID NO: 164
   LC-CDR3 having the amino acid sequence of SEQ ID NO: 194.

In some embodiments the antigen-binding molecule which is capable of binding to CD122 comprises:
a VH region comprising an amino acid sequence having at least 85% sequence identity to one of SEQ ID NOs:1 to 34; and
a VL region comprising an amino acid sequence having at least 85% sequence identity to one of SEQ ID NOS:35 to 65.

In some embodiments the antigen-binding molecule which is capable of binding to CD122 comprises: (P2C4) a VH region comprising an amino acid sequence having at least 85% sequence identity to SEQ ID NO: 1; and
a VL region comprising an amino acid sequence having at least 85% sequence identity to SEQ ID NO: 35; or
(P2C4_A4) a VH region comprising an amino acid sequence having at least 85% sequence identity to SEQ ID NO: 1; and
a VL region comprising an amino acid sequence having at least 85% sequence identity to SEQ ID NO: 39; or
(P2C₄₋₈₁) a VH region comprising an amino acid sequence having at least 85% sequence identity to SEQ ID NO: 1; and
a VL region comprising an amino acid sequence having at least 85% sequence identity to SEQ ID NO: 40; or
(P2C₄₋₈₅) a VH region comprising an amino acid sequence having at least 85% sequence identity to SEQ ID NO: 1; and
a VL region comprising an amino acid sequence having at least 85% sequence identity to SEQ ID NO: 41; or
(P2C4_C4) a VH region comprising an amino acid sequence having at least 85% sequence identity to SEQ ID NO: 1; and
a VL region comprising an amino acid sequence having at least 85% sequence identity to SEQ ID NO: 44; or
(P2C4_C7) a VH region comprising an amino acid sequence having at least 85% sequence identity to SEQ ID NO: 1; and
a VL region comprising an amino acid sequence having at least 85% sequence identity to SEQ ID NO: 45; or
(P2C4_D10) a VH region comprising an amino acid sequence having at least 85% sequence identity to SEQ ID NO: 1; and
a VL region comprising an amino acid sequence having at least 85% sequence identity to SEQ ID NO: 42; or
(P2C4_E6) a VH region comprising an amino acid sequence having at least 85% sequence identity to SEQ ID NO: 1; and
a VL region comprising an amino acid sequence having at least 85% sequence identity to SEQ ID NO: 46; or
(P2C4_E7) a VH region comprising an amino acid sequence having at least 85% sequence identity to SEQ ID NO: 1; and
a VL region comprising an amino acid sequence having at least 85% sequence identity to SEQ ID NO: 47; or
(P2C4_F8) a VH region comprising an amino acid sequence having at least 85% sequence identity to SEQ ID NO: 1; and
a VL region comprising an amino acid sequence having at least 85% sequence identity to SEQ ID NO: 49; or
(P2H7) a VH region comprising an amino acid sequence having at least 85% sequence identity to SEQ ID NO: 2; and
a VL region comprising an amino acid sequence having at least 85% sequence identity to SEQ ID NO: 36; or
(P2D12) a VH region comprising an amino acid sequence having at least 85% sequence identity to SEQ ID NO: 3; and
a VL region comprising an amino acid sequence having at least 85% sequence identity to SEQ ID NO: 37; or
(P1G11) a VH region comprising an amino acid sequence having at least 85% sequence identity to SEQ ID NO: 4; and
a VL region comprising an amino acid sequence having at least 85% sequence identity to SEQ ID NO: 38; or
(P2C4_A9) a VH region comprising an amino acid sequence having at least 85% sequence identity to SEQ ID NO: 5; and
a VL region comprising an amino acid sequence having at least 85% sequence identity to SEQ ID NO: 35; or
(P2C4_B6) a VH region comprising an amino acid sequence having at least 85% sequence identity to SEQ ID NO: 6; and
a VL region comprising an amino acid sequence having at least 85% sequence identity to SEQ ID NO: 42; or
(P2C4_E9) a VH region comprising an amino acid sequence having at least 85% sequence identity to SEQ ID NO: 6; and
a VL region comprising an amino acid sequence having at least 85% sequence identity to SEQ ID NO: 48; or
(P2C4_B8) a VH region comprising an amino acid sequence having at least 85% sequence identity to SEQ ID NO: 7; and
a VL region comprising an amino acid sequence having at least 85% sequence identity to SEQ ID NO: 42; or
(P2C4_B12) a VH region comprising an amino acid sequence having at least 85% sequence identity to SEQ ID NO: 8; and
a VL region comprising an amino acid sequence having at least 85% sequence identity to SEQ ID NO: 43; or
(P2C4_C1) a VH region comprising an amino acid sequence having at least 85% sequence identity to SEQ ID NO: 9; and
a VL region comprising an amino acid sequence having at least 85% sequence identity to SEQ ID NO: 39; or
(P2C4_C12) a VH region comprising an amino acid sequence having at least 85% sequence identity to SEQ ID NO: 10; and
a VL region comprising an amino acid sequence having at least 85% sequence identity to SEQ ID NO: 42; or
(P2C4_E2) a VH region comprising an amino acid sequence having at least 85% sequence identity to SEQ ID NO: 11; and
a VL region comprising an amino acid sequence having at least 85% sequence identity to SEQ ID NO: 42; or
(P2C4_E3) a VH region comprising an amino acid sequence having at least 85% sequence identity to SEQ ID NO: 12; and
a VL region comprising an amino acid sequence having at least 85% sequence identity to SEQ ID NO: 42; or
(P2C4_E8) a VH region comprising an amino acid sequence having at least 85% sequence identity to SEQ ID NO: 13; and
a VL region comprising an amino acid sequence having at least 85% sequence identity to SEQ ID NO: 42; or
(P2C4_F11) a VH region comprising an amino acid sequence having at least 85% sequence identity to SEQ ID NO: 14; and
a VL region comprising an amino acid sequence having at least 85% sequence identity to SEQ ID NO: 50; or
(P2C4_G2) a VH region comprising an amino acid sequence having at least 85% sequence identity to SEQ ID NO: 15; and
a VL region comprising an amino acid sequence having at least 85% sequence identity to SEQ ID NO: 42; or
(P2C4_G11) a VH region comprising an amino acid sequence having at least 85% sequence identity to SEQ ID NO: 16; and
a VL region comprising an amino acid sequence having at least 85% sequence identity to SEQ ID NO: 42; or
(P2C4_H1) a VH region comprising an amino acid sequence having at least 85% sequence identity to SEQ ID NO: 17; and
a VL region comprising an amino acid sequence having at least 85% sequence identity to SEQ ID NO: 42; or
(P2C4_H2) a VH region comprising an amino acid sequence having at least 85% sequence identity to SEQ ID NO: 18; and
a VL region comprising an amino acid sequence having at least 85% sequence identity to SEQ ID NO: 42; or
(P2C4_H3) a VH region comprising an amino acid sequence having at least 85% sequence identity to SEQ ID NO: 19; and
a VL region comprising an amino acid sequence having at least 85% sequence identity to SEQ ID NO: 42; or
(P2C4_C1 D10) a VH region comprising an amino acid sequence having at least 85% sequence identity to SEQ ID NO: 20; and
a VL region comprising an amino acid sequence having at least 85% sequence identity to SEQ ID NO: 51; or
(P2C4_FW2) a VH region comprising an amino acid sequence having at least 85% sequence identity to SEQ ID NO: 21; and
a VL region comprising an amino acid sequence having at least 85% sequence identity to SEQ ID NO: 52; or
(P1E7) a VH region comprising an amino acid sequence having at least 85% sequence identity to SEQ ID NO: 22; and
a VL region comprising an amino acid sequence having at least 85% sequence identity to SEQ ID NO: 53; or
(P1 B10) a VH region comprising an amino acid sequence having at least 85% sequence identity to SEQ ID NO: 23; and
a VL region comprising an amino acid sequence having at least 85% sequence identity to SEQ ID NO: 54; or
(P1 F3) a VH region comprising an amino acid sequence having at least 85% sequence identity to SEQ ID NO: 24; and
a VL region comprising an amino acid sequence having at least 85% sequence identity to SEQ ID NO: 55; or
(P1010) a VH region comprising an amino acid sequence having at least 85% sequence identity to SEQ ID NO: 25; and
a VL region comprising an amino acid sequence having at least 85% sequence identity to SEQ ID NO: 56; or
(P1E1) a VH region comprising an amino acid sequence having at least 85% sequence identity to SEQ ID NO: 26; and
a VL region comprising an amino acid sequence having at least 85% sequence identity to SEQ ID NO: 57; or
(P2B11) a VH region comprising an amino acid sequence having at least 85% sequence identity to SEQ ID NO: 27; and
a VL region comprising an amino acid sequence having at least 85% sequence identity to SEQ ID NO: 58; or
(P2C9) a VH region comprising an amino acid sequence having at least 85% sequence identity to SEQ ID NO: 28; and
a VL region comprising an amino acid sequence having at least 85% sequence identity to SEQ ID NO: 59; or
(P2C10) a VH region comprising an amino acid sequence having at least 85% sequence identity to SEQ ID NO: 29; and
a VL region comprising an amino acid sequence having at least 85% sequence identity b SEQ ID NO: 60; or
(P2C11) a VH region comprising an amino acid sequence having at least 85% sequence identity to SEQ ID NO: 30; and
a VL region comprising an amino acid sequence having at least 85% sequence identity to SEQ ID NO: 61; or
(P2E6) a VH region comprising an amino acid sequence having at least 85% sequence identity to SEQ ID NO: 31; and
a VL region comprising an amino acid sequence having at least 85% sequence identity to SEQ ID NO: 62; or
(P2E11) a VH region comprising an amino acid sequence having at least 85% sequence identity to SEQ ID NO: 32; and
a VL region comprising an amino acid sequence having at least 85% sequence identity to SEQ ID NO: 63; or
(P2F9) a VH region comprising an amino acid sequence having at least 85% sequence identity to SEQ ID NO: 33; and
a VL region comprising an amino acid sequence having at least 85% sequence identity b SEQ ID NO: 64; or

(P2F10) a VH region comprising an amino acid sequence having at least 85% sequence identity to SEQ ID NO: 34; and
a VL region comprising an amino acid sequence having at least 85% sequence identity to SEQ ID NO: 65.

In some embodiments the antigen-binding molecule which is capable of binding b CD132 comprises:
(P1A10) a VH region incorporating the following CDRS:
   HC-CDR1 having the amino acid sequence of SEQ ID NO: 196
   HC-CDR2 having the amino acid sequence of SEQ ID NO: 204
   HC-CDR3 having the amino acid sequence of SEQ ID NO: 212; and
a VL region incorporating the following CDRS:
   LC-CDR1 having the amino acid sequence of SEQ ID NO: 227
   LC-CDR2 having the amino acid sequence of SEQ ID NO: 238
   LC-CDR3 having the amino acid sequence of SEQ ID NO: 248; or
(P186) a VH region incorporating the following CDRS:
   HC-CDR1 having the amino acid sequence of SEQ ID NO: 108
   HC-CDR2 having the amino acid sequence of SEQ ID NO: 120
   HC-CDR3 having the amino acid sequence of SEQ ID NO: 213; and
a VL region incorporating the following CDRS:
   LC-CDR1 having the amino acid sequence of SEQ ID NO: 151
   LC-CDR2 having the amino acid sequence of SEQ ID NO: 239
   LC-CDR3 having the amino acid sequence of SEQ ID NO: 249; or
(P1C10) a VH region incorporating the following CDRS:
   HC-CDR1 having the amino acid sequence of SEQ ID NO: 112
   HC-CDR2 having the amino acid sequence of SEQ ID NO: 124
   HC-CDR3 having the amino acid sequence of SEQ ID NO: 214; and
a VL region incorporating the following CDRS:
   LC-CDR1 having the amino acid sequence of SEQ ID NO: 228
   LC-CDR2 having the amino acid sequence of SEQ ID NO: 240
   LC-CDR3 having the amino acid sequence of SEQ ID NO: 250; or
(P107) a VH region incorporating the following CDRs:
   HC-CDR1 having the amino acid sequence of SEQ ID NO: 197
   HC-CDR2 having the amino acid sequence of SEQ ID NO: 206
   HC-CDR3 having the amino acid sequence of SEQ ID NO: 215; and
a VL region incorporating the following CDRS:
   LC-CDR1 having the amino acid sequence of SEQ ID NO: 229
   LC-CDR2 having the amino acid sequence of SEQ ID NO: 241
   LC-CDR3 having the amino acid sequence of SEQ ID NO: 251; or
(P1E8) a VH region incorporating the following CDRS:
   HC-CDR1 having the amino acid sequence of SEQ ID NO: 198
   HC-CDR2 having the amino acid sequence of SEQ ID NO: 120
   HC-CDR3 having the amino acid sequence of SEQ ID NO: 216; and
a VL region incorporating the following CDRS:
   LC-CDR1 having the amino acid sequence of SEQ ID NO: 230
   LC-CDR2 having the amino acid sequence of SEQ ID NO: 242
   LC-CDR3 having the amino acid sequence of SEQ ID NO: 252; or
(P2B2) a VH region incorporating the following CDRS:
   HC-CDR1 having the amino acid sequence of SEQ ID NO: 108
   HC-CDR2 having the amino acid sequence of SEQ ID NO: 207
   HC-CDR3 having the amino acid sequence of SEQ ID NO: 217; and
a VL region incorporating the following CDRs:
   LC-CDR1 having the amino acid sequence of SEQ ID NO: 151
   LC-CDR2 having the amino acid sequence of SEQ ID NO: 174
   LC-CDR3 having the amino acid sequence of SEQ ID NO: 253; or
(P2B7) a VH region incorporating the following CDRs:
   HC-CDR1 having the amino acid sequence of SEQ ID NO: 106
   HC-CDR2 having the amino acid sequence of SEQ ID NO: 119
   HC-CDR3 having the amino acid sequence of SEQ ID NO: 218; and
a VL region incorporating the following CDRs:
   LC-CDR1 having the amino acid sequence of SEQ ID NO: 231
   LC-CDR2 having the amino acid sequence of SEQ ID NO: 174
   LC-CDR3 having the amino acid sequence of SEQ ID NO: 254; or
(P2D11) a VH region incorporating the following CDRs:
   HC-CDR1 having the amino acid sequence of SEQ ID NO: 199
   HC-CDR2 having the amino acid sequence of SEQ ID NO: 208
   HC-CDR3 having the amino acid sequence of SEQ ID NO: 219; and
a VL region incorporating the following CDRs:
   LC-CDR1 having the amino acid sequence of SEQ ID NO: 232
   LC-CDR2 having the amino acid sequence of SEQ ID NO: 243
   LC-CDR3 having the amino acid sequence of SEQ ID NO: 255; or
(P2F10) a VH region incorporating the following CDRs:
   HC-CDR1 having the amino acid sequence of SEQ ID NO: 200
   HC-CDR2 having the amino acid sequence of SEQ ID NO: 209
   HC-CDR3 having the amino acid sequence of SEQ ID NO: 220; and
a VL region incorporating the following CDRs:
   LC-CDR1 having the amino acid sequence of SEQ ID NO: 233
   LC-CDR2 having the amino acid sequence of SEQ ID NO: 244
   LC-CDR3 having the amino acid sequence of SEQ ID NO: 256; or
(P2H4) a VH region incorporating the following CD Rs:
   HC-CDR1 having the amino acid sequence of SEQ ID NO: 108
   HC-CDR2 having the amino acid sequence of SEQ ID NO: 120
   HC-CDR3 having the amino acid sequence of SEQ ID NO: 221; and
a VL region incorporating the following CDRs:
   LC-CDR1 having the amino acid sequence of SEQ ID NO: 234
   LC-CDR2 having the amino acid sequence of SEQ ID NO: 174
   LC-CDR3 having the amino acid sequence of SEQ ID NO: 257; or
(P2D3) a VH region incorporating the following CDRs:
   HC-CDR1 having the amino acid sequence of SEQ ID NO: 201
   HC-CDR2 having the amino acid sequence of SEQ ID NO: 119
   HC-CDR3 having the amino acid sequence of SEQ ID NO: 222; and
a VL region incorporating the following CDRs:
   LC-CDR1 having the amino acid sequence of SEQ ID NO: 151
   LC-CDR2 having the amino acid sequence of SEQ ID NO: 174
   LC-CDR3 having the amino acid sequence of SEQ ID NO: 189; or
(P1G4) a VH region incorporating the following CDRs:
   HC-CDR1 having the amino acid sequence of SEQ ID NO: 106
   HC-CDR2 having the amino acid sequence of SEQ ID NO: 119
   HC-CDR3 having the amino acid sequence of SEQ ID NO: 223; and
a VL region incorporating the following CDRs:
   LC-CDR1 having the amino acid sequence of SEQ ID NO: 151
   LC-CDR2 having the amino acid sequence of SEQ ID NO: 174
   LC-CDR3 having the amino acid sequence of SEQ ID NO: 258; or
(P1 B12) a VH region incorporating the following CDRs:
   HC-CDR1 having the amino acid sequence of SEQ ID NO: 106
   HC-CDR2 having the amino acid sequence of SEQ ID NO: 119
   HC-CDR3 having the amino acid sequence of SEQ ID NO: 224; and
a VL region incorporating the following CDRs:
   LC-CDR1 having the amino acid sequence of SEQ ID NO: 235
   LC-CDR2 having the amino acid sequence of SEQ ID NO: 174
   LC-CDR3 having the amino acid sequence of SEQ ID NO: 189; or
(P1C7) a VH region incorporating the following CDRs:
   HC-CDR1 having the amino acid sequence of SEQ ID NO: 106
   HC-CDR2 having the amino acid sequence of SEQ ID NO: 119
   HC-CDR3 having the amino acid sequence of SEQ ID NO: 225; and
a VL region incorporating the following CDRs:
   LC-CDR1 having the amino acid sequence of SEQ ID NO: 151
   LC-CDR2 having the amino acid sequence of SEQ ID NO: 245
   LC-CDR3 having the amino acid sequence of SEQ ID NO: 189.

In some embodiments the antigen-binding molecule which is capable of binding to CD132 comprises:
a VH region comprising an amino acid sequence having at least 85% sequence identity to one of SEQ ID NOS:71 to 84; and
a VL region comprising an amino acid sequence having at least 85% sequence identity to one of SEQ ID NOs:89 to 102.

In some embodiments the antigen-binding molecule which is capable of binding to CD132 comprises:
(P1A10) a VH region comprising an amino acid sequence having at least 85% sequence identity to SEQ ID NO: 71; and
a VL region comprising an amino acid sequence having at least 85% sequence identity to SEQ ID NO: 89; or
(P1 B6) a VH region comprising an amino acid sequence having at least 85% sequence identity to SEQ ID NO: 72; and
a VL region comprising an amino acid sequence having at least 85% sequence identity to SEQ ID NO: 90; or
(P1C10) a VH region comprising an amino acid sequence having at least 85% sequence identity to SEQ ID NO: 73; and
a VL region comprising an amino acid sequence having at least 85% sequence identity to SEQ ID NO: 91; or
(P107) a VH region comprising an amino acid sequence having at least 85% sequence identity to SEQ ID NO: 74; and
a VL region comprising an amino acid sequence having at least 85% sequence identity to SEQ ID NO: 92; or
(P1 E8) a VH region comprising an amino acid sequence having at least 85% sequence identity to SEQ ID NO: 75; and
a VL region comprising an amino acid sequence having at least 85% sequence identity to SEQ ID NO: 93; or
(P2B2) a VH region comprising an amino acid sequence having at least 85% sequence identity to SEQ ID NO: 76; and
a VL region comprising an amino acid sequence having at least 85% sequence identity to SEQ ID NO: 94; or
(P2B7) a VH region comprising an amino acid sequence having at least 85% sequence identity to SEQ ID NO: 77; and
a VL region comprising an amino acid sequence having at least 85% sequence identity to SEQ ID NO: 95; or
(P2011) a VH region comprising an amino acid sequence having at least 85% sequence identity to SEQ ID NO: 78; and
a VL region comprising an amino acid sequence having at least 85% sequence identity to SEQ ID NO: 96; or
(P2F10) a VH region comprising an amino acid sequence having at least 85% sequence identity to SEQ ID NO: 79; and
a VL region comprising an amino acid sequence having at least 85% sequence identity to SEQ ID NO: 97; or
(P2H4) a VH region comprising an amino acid sequence having at least 85% sequence identity to SEQ ID NO: 80; and
a VL region comprising an amino acid sequence having at least 85% sequence identity to SEQ ID NO: 98; or
(P203) a VH region comprising an amino acid sequence having at least 85% sequence identity to SEQ ID NO: 81; and
a VL region comprising an amino acid sequence having at least 85% sequence identity to SEQ ID NO: 99; or
(P1 G4) a VH region comprising an amino acid sequence having at least 85% sequence identity to SEQ ID NO: 82; and
a VL region comprising an amino acid sequence having at least 85% sequence identity to SEQ ID NO: 100; or
(P1B12) a VH region comprising an amino acid sequence having at least 85% sequence identity to SEQ ID NO: 83; and
a VL region comprising an amino acid sequence having at least 85% sequence identity to SEQ ID NO: 101; or
(P1C7) a VH region comprising an amino acid sequence having at least 85% sequence identity to SEQ ID NO: 84; and
a VL region comprising an amino acid sequence having at least 85% sequence identity to SEQ ID NO: 102.

The present invention also provides an antigen-binding molecule, optionally isolated, which is capable of binding to CD122, comprising:
a heavy chain variable (VH) region incorporating the following CDRS:
   HC-CDR1 having the amino acid sequence of one of SEQ ID NOs:105, 106, or 108 to 115
   HC-CDR2 having the amino acid sequence of one of SEQ ID NOs:119 to 127
   HC-CDR3 having the amino acid sequence of one of SEQ ID NOs:133 to 144; and
a light chain variable (VL) region incorporating the following CDRs:
   LC-CDR1 having the amino acid sequence of one of SEQ ID NOs:151 to 161
   LC-CDR2 having the amino acid sequence of one of SEQ ID NOs: 164, or 169 to 176
   LC-CDR3 having the amino acid sequence of one of SEQ ID NOs:182 to 194;
or a variant thereof in which one or two or three amino acids in one or more of HC-CDR 1, HC-CDR2, HC-CDR3, LC-CDR1, LC-CDR2 or LC-CDR3 are substituted with another amino acid.

In some embodiments the antigen-binding molecule comprises:
(P1 E7) a VH region incorporating the following CDRs:
   HC-CDR1 having the amino acid sequence of SEQ ID NO: 108
   HC-CDR2 having the amino acid sequence of SEQ ID NO: 120
   HC-CDR3 having the amino acid sequence of SEQ ID NO: 133; and
a VL region incorporating the following CDRs:
   LC-CDR1 having the amino acid sequence of SEQ ID NO: 151
   LC-CDR2 having the amino acid sequence of SEQ ID NO: 169
   LC-CDR3 having the amino acid sequence of SEQ ID NO: 182; or
(P1 B10) a VH region incorporating the following CDRs:
   HC-CDR1 having the amino acid sequence of SEQ ID NO: 109
   HC-CDR2 having the amino acid sequence of SEQ ID NO: 121
   HC-CDR3 having the amino acid sequence of SEQ ID NO: 134; and
a VL region incorporating the following CDRs:
   LC-CDR1 having the amino acid sequence of SEQ ID NO: 152
   LC-CDR2 having the amino acid sequence of SEQ ID NO: 164
   LC-CDR3 having the amino acid sequence of SEQ ID NO: 183; or
(P1F3) a VH region incorporating the following CDRs:
   HC-CDR1 having the amino acid sequence of SEQ ID NO: 105
   HC-CDR2 having the amino acid sequence of SEQ ID NO: 122
   HC-CDR3 having the amino acid sequence of SEQ ID NO: 135; and
a VL region incorporating the following CDRs:
   LC-CDR1 having the amino acid sequence of SEQ ID NO: 153
   LC-CDR2 having the amino acid sequence of SEQ ID NO: 164
   LC-CDR3 having the amino acid sequence of SEQ ID NO 184; or
(P1D10) a VH region incorporating the following CDRs:
   HC-CDR 1 having the amino acid sequence of SEQ ID NO: 110
   HC-CDR2 having the amino acid sequence of SEQ ID NO: 119
   HC-CDR3 having the amino acid sequence of SEQ ID NO: 136; and
a VL region incorporating the following CDRs:
   LC-CDR1 having the amino acid sequence of SEQ ID NO. 154
   LC-CDR2 having the amino acid sequence of SEQ ID NO: 170
   LC-CDR3 having the amino acid sequence of SEQ ID NO: 185; or
(P1E1) a VH region incorporating the following CDRs:
   HC-CDR1 having the amino acid sequence of SEQ ID NO: 106
   HC-CDR2 having the amino acid sequence of SEQ ID NO: 119
   HC-CDR3 having the amino acid sequence of SEQ ID NO: 137; and
a VL region incorporating the following CDRs:
   LC-CDR1 having the amino acid sequence of SEQ ID NO: 155
   LC-CDR2 having the amino acid sequence of SEQ ID NO: 171
   LC-CDR3 having the amino acid sequence of SEQ ID NO: 186; or
(P2B11) a VH region incorporating the following CDRs:
   HC-CDR1 having the amino acid sequence of SEO ID NO: 111
   HC-CDR2 having the amino acid sequence of SEQ ID NO: 123
   HC-CDR3 having the amino acid sequence of SEQ ID NO: 138; and
a VL region incorporating the following CDRs:
   LC-CDR1 having the amino acid sequence of SEQ ID NO: 156
   LC-CDR2 having the amino acid sequence of SEQ ID NO: 172
   LC-CDR3 having the amino acid sequence of SEQ ID NO 187; or
(P209) a VH region incorporating the following CDRs:
   HC-CDR1 having the amino acid sequence of SEQ ID NO: 112
   HC-CDR2 having the amino acid sequence of SEQ ID NO: 124
   HC-CDR3 having the amino acid sequence of SEQ ID NO: 139; and
a VL region incorporating the following GDRs:
   LC-CDR1 having the amino acid sequence of SEQ ID NO: 157
   LC-CDR2 having the amino acid sequence of SEQ ID NO: 173
   LC-CDR3 having the amino acid sequence of SEQ ID NO: 188; or
(P2C10) a VH region incorporating the following CDRs:
   HC-CDR1 having the amino acid sequence of SEQ ID NO: 106
   HC-CDR2 having the amino acid sequence of SEQ ID NO: 119
   HC-CDR3 having the amino acid sequence of SEQ ID NO: 140; and
a VL region incorporating the following CD Rs:
   LC-CDR1 having the amino acid sequence of SEQ ID NO: 158
   LC-CDR2 having the amino acid sequence of SEQ ID NO: 174
   LC-CDR3 having the amino acid sequence of SEQ ID NO: 189; or
(P2C11) a VH region incorporating the following CD Rs:
   HC-CDR1 having the amino acid sequence of SEQ ID NO: 113
   HC-CDR2 having the amino acid sequence of SEQ ID NO: 125
   HC-CDR3 having the amino acid sequence of SEQ ID NO: 141; and
a VL region incorporating the following CD Rs:
   LC-CDR1 having the amino acid sequence of SEQ ID NO: 159
   LC-CDR2 having the amino acid sequence of SEQ ID NO: 175
   LC-CDR3 having the amino acid sequence of SEQ ID NO: 190; or
(P2E6) a VH region incorporating the following CDRs:
   HC-CDR1 having the amino acid sequence of SEQ ID NO: 114
   HC-CDR2 having the amino acid sequence of SEQ ID NO: 126
   HC-CDR3 having the amino acid sequence of SEQ ID NO: 142; and
a VL region incorporating the following CD Rs:
   LC-CDR1 having the amino acid sequence of SEQ ID NO: 160
   LC-CDR2 having the amino acid sequence of SEQ ID NO: 176
   LC-CDR3 having the amino acid sequence of SEQ ID NO: 191; or
(P2E11) a VH region incorporating the following CDRs:
   HC-CDR1 having the amino acid sequence of SEQ ID NO: 109
   HC-CDR2 having the amino acid sequence of SEQ ID NO: 121
   HC-CDR3 having the amino acid sequence of SEQ ID NO: 134; and
a VL region incorporating the following CD Rs:
   LC-CDR1 having the amino acid sequence of SEQ ID NO: 159
   LC-CDR2 having the amino acid sequence of SEQ ID NO: 164
   LC-CDR3 having the amino acid sequence of SEQ ID NO: 192; or
(P2F9) a VH region incorporating the following CDRs:
   HC-CDR1 having the amino acid sequence of SEQ ID NO: 115
   HC-CDR2 having the amino acid sequence of SEQ ID NO: 127
   HC-CDR3 having the amino acid sequence of SEQ ID NO: 143; and
a VL region incorporating the following CD Rs:
   LC-CDR1 having the amino acid sequence of SEQ ID NO: 151
   LC-CDR2 having the amino acid sequence of SEQ ID NO: 174
   LC-CDR3 having the amino acid sequence of SEQ ID NO: 193; or
(P2F10) a VH region incorporating the following CDRs:
   HC-CDR1 having the amino acid sequence of SEQ ID NO: 115
   HC-CDR2 having the amino acid sequence of SEQ ID NO: 127
   HC-CDR3 having the amino acid sequence of SEQ ID NO: 144; and
a VL region incorporating the following CDRs:
   LC-CDR 1 having the amino acid sequence of SEQ ID NO: 161
   LC-CDR2 having the amino acid sequence of SEQ ID NO: 164
   LC-CDR3 having the amino acid sequence of SEQ ID NO: 194.

In some embodiments the antigen-binding molecule comprises:
a VH region comprising an amino acid sequence having at least 85% sequence identity to one of SEQ D NOs:22 to 34; and
a VL region comprising an amino acid sequence having at least 85% sequence identity to one of SEQ ID NOs:53 to 65.

In some embodiments the antigen-binding molecule comprises:
(P1 E7) a VH region comprising an amino acid sequence having at least 85% sequence identity to SEQ ID NO: 22; and
a VL region comprising an amino acid sequence having at least 85% sequence identity to SEQ ID NO: 53; or
(P1 B10) a VH region comprising an amino acid sequence having at least 85% sequence identity to SEQ ID NO: 23; and
a VL region comprising an amino acid sequence having at least 85% sequence identity to SEQ ID NO: 54; or
(P1 F3) a VH region comprising an amino acid sequence having at least 85% sequence identity to SEQ ID NO: 24; and
a VL region comprising an amino acid sequence having at least 85% sequence identity to SEQ ID NO: 55; or
(P1010) a VH region comprising an amino acid sequence having at least 85% sequence identity to SEQ ID NO: 25; and
a VL region comprising an amino acid sequence having at least 85% sequence identity to SEQ ID NO: 56; or
(P1 E1) a VH region comprising an amino acid sequence having at least 85% sequence identity to SEQ ID NO: 26; and
a VL region comprising an amino acid sequence having at least 85% sequence identity to SEQ ID NO: 57; or
(P2B11) a VH region comprising an amino acid sequence having at least 85% sequence identity to SEQ ID NO: 27; and
a VL region comprising an amino acid sequence having at least 85% sequence identity to SEQ ID NO: 58; or
(P2C9) a VH region comprising an amino acid sequence having at least 85% sequence identity to SEQ ID NO: 28; and
a VL region comprising an amino acid sequence having at least 85% sequence identity to SEQ ID NO: 59; or
(P2C10) a VH region comprising an amino acid sequence having at least 85% sequence identity to SEQ ID NO: 29; and
a VL region comprising an amino acid sequence having at least 85% sequence identity to SEQ ID NO: 60; or
(P2C11) a VH region comprising an amino acid sequence having at least 85% sequence identity to SEQ ID NO: 30; and
a VL region comprising an amino acid sequence having at least 85% sequence identity to SEQ ID NO: 61; or
(P2E6) a VH region comprising an amino acid sequence having at least 85% sequence identity to SEQ ID NO: 31; and
a VL region comprising an amino acid sequence having at least 85% sequence identity to SEQ ID NO: 62; or
(P2E11) a VH region comprising an amino acid sequence having at least 85% sequence identity to SEQ ID NO: 32; and
a VL region comprising an amino acid sequence having at least 85% sequence identity to SEQ ID NO: 63; or
(P2F9) a VH region comprising an amino acid sequence having at least 85% sequence identity to SEQ ID NO: 33; and
a VL region comprising an amino acid sequence having at least 85% sequence identity to SEQ ID NO: 64; or
(P2F10) a VH region comprising an amino acid sequence having at least 85% sequence identity to SEQ ID NO: 34; and
a VL region comprising an amino acid sequence having at least 85% sequence identity to SEQ ID NO: 65.

The present invention also provides an antigen-binding molecule, optionally isolated, which is capable of binding to CD132, comprising:
a heavy chain variable (VH) region incorporating the following CDRs:
   HC-CDR 1 having the amino acid sequence of one of SEQ ID NOs: 106, 108, 112, or 196 to 201
   HC-CDR2 having the amino acid sequence of one of SEQ ID NOs:119, 120, 124, or 204 to 209
   HC-CDR3 having the amino acid sequence of one of SEQ ID NOs:212 to 225; and
a light chain variable (VL) region incorporating the following CDRs:
   LC-CDR1 having the amino acid sequence of one of SEQ ID NOs:151, or 227 to 235
   LC-CDR2 having the amino acid sequence of one of SEQ ID NOS:174, or 238 to 245
   LC-CDR3 having the amino acid sequence of one of SEQ ID NOs: 189, or 248 to 258;
or a variant thereof in which one or two or three amino acids in one or more of HC-CDR1, HC-CDR2, HC-CDR3, LC-CDR1, LC-CDR2 or LC-CDR3 are substituted with another amino acid.

In some embodiments the antigen-binding molecule comprises:
(P1A10) a VH region incorporating the following CDRs:
   HC-CDR1 having the amino acid sequence of SEQ ID NO: 196
   HC-CDR2 having the amino acid sequence of SEQ ID NO: 204
   HC-CDR3 having the amino acid sequence of SEQ ID NO: 212; and
a VL region incorporating the following CD Rs:
   LC-CDR1 having the amino acid sequence of SEQ ID NO: 227
   LC-CDR2 having the amino acid sequence of SEQ ID NO: 238
   LC-CDR3 having the amino acid sequence of SEQ ID NO: 248; or
(P1B6) a VH region incorporating the following CDRs:
   HC-CDR1 having the amino acid sequence of SEQ ID NO: 108
   HC-CDR2 having the amino acid sequence of SEQ ID NO: 120
   HC-CDR3 having the amino acid sequence of SEQ ID NO: 213; and
a VL region incorporating the following CD Rs:
   LC-CDR1 having the amino acid sequence of SEQ ID NO: 151
   LC-CDR2 having the amino acid sequence of SEQ ID NO: 239
   LC-CDR3 having the amino acid sequence of SEQ ID NO: 249; or
(P1C10) a VH region incorporating the following CDRs:
   HC-CDR1 having the amino acid sequence of SEQ ID NO: 112
   HC-CDR2 having the amino acid sequence of SEQ ID NO: 124
   HC-CDR3 having the amino acid sequence of SEQ ID NO: 214; and
a VL region incorporating the following CD Rs:
   LC-CDR1 having the amino acid sequence of SEQ ID NO: 228
   LC-CDR2 having the amino acid sequence of SEQ ID NO: 240
   LC-CDR3 having the amino acid sequence of SEQ ID NO: 250; or
(P107) a VH region incorporating the following CDRs:
   HC-CDR1 having the amino acid sequence of SEQ ID NO: 197
   HC-CDR2 having the amino acid sequence of SEQ ID NO: 206
   HC-CDR3 having the amino acid sequence of SEQ ID NO: 215; and
a VL region incorporating the following CD Rs:
   LC-CDR1 having the amino acid sequence of SEQ ID NO: 229
   LC-CDR2 having the amino acid sequence of SEQ ID NO: 241
   LC-CDR3 having the amino acid sequence of SEQ ID NO: 251; or
(P1E8) a VH region incorporating the following CDRs:
   HC-CDR1 having the amino acid sequence of SEQ ID NO: 198
   HC-CDR2 having the amino acid sequence of SEQ ID NO: 120
   HC-CDR3 having the amino acid sequence of SEQ ID NO: 216; and
a VL region incorporating the following CD Rs:
   LC-CDR1 having the amino acid sequence of SEQ ID NO: 230
   LC-CDR2 having the amino acid sequence of SEQ ID NO: 242
   LC-CDR3 having the amino acid sequence of SEQ ID NO: 252; or
(P2B2) a VH region incorporating the following CDRs:
   HC-CDR1 having the amino acid sequence of SEQ ID NO: 108
   HC-CDR2 having the amino acid sequence of SEQ ID NO: 207
   HC-CDR3 having the amino acid sequence of SEQ ID NO: 217; and
a VL region incorporating the following CDRs:
   LC-CDR1 having the amino acid sequence of SEQ ID NO: 151
   LC-CDR2 having the amino acid sequence of SEQ ID NO: 174
   LC-CDR3 having the amino acid sequence of SEQ ID NO: 253; or
(P2B7) a VH region incorporating the following CDRs:
   HC-CDR1 having the amino acid sequence of SEQ ID NO: 106
   HC-CDR2 having the amino acid sequence of SEQ ID NO: 119
   HC-CDR3 having the amino acid sequence of SEQ ID NO: 218; and
a VL region incorporating the following CDRs:
   LC-CDR1 having the amino acid sequence of SEQ ID NO: 231
   LC-CDR2 having the amino acid sequence of SEQ ID NO: 174
   LC-CDR3 having the amino acid sequence of SEQ ID NO: 254; or
(P2D11) a VH region incorporating the following CDRs:
   HC-CDR1 having the amino acid sequence of SEQ ID NO: 199
   HC-CDR2 having the amino acid sequence of SEQ ID NO: 208
   HC-CDR3 having the amino acid sequence of SEQ ID NO: 219; and
a VL region incorporating the following CDRs:
   LC-CDR1 having the amino acid sequence of SEQ ID NO: 232
   LC-CDR2 having the amino acid sequence of SEQ ID NO: 243
   LC-CDR3 having the amino acid sequence of SEQ ID NO: 255; or
(P2F10) a VH region incorporating the following CDRS:
   HC-CDR1 having the amino acid sequence of SEQ ID NO: 200
   HC-CDR2 having the amino acid sequence of SEQ ID NO: 209
   HC-CDR3 having the amino acid sequence of SEQ ID NO: 220; and
a VL region incorporating the following CDRs:
   LC-CDR1 having the amino acid sequence of SEQ ID NO: 233
   LC-CDR2 having the amino acid sequence of SEQ ID NO: 244
   LC-CDR3 having the amino acid sequence of SEQ ID NO: 256; or
(P2H4) a VH region incorporating the following CDRs:
   HC-CDR 1 having the amino acid sequence of SEQ ID NO: 108
   HC-CDR2 having the amino acid sequence of SEQ ID NO: 120
   HC-CDR3 having the amino acid sequence of SEQ ID NO: 221; and
a VL region incorporating the following CDRs:
   LC-CDR1 having the amino acid sequence of SEQ ID NO: 234
   LC-CDR2 having the amino acid sequence of SEQ ID NO: 174
   LC-CDR3 having the amino acid sequence of SEQ ID NO: 257; or
(P2D3) a VH region incorporating the following CDRs:
   HC-CDR1 having the amino acid sequence of SEQ ID NO: 201
   HC-CDR2 having the amino acid sequence of SEQ ID NO: 119
   HC-CDR3 having the amino acid sequence of SEQ ID NO: 222; and
a VL region incorporating the following CDRs:
   LC-CDR1 having the amino acid sequence of SEQ ID NO: 151
   LC-CDR2 having the amino acid sequence of SEQ ID NO: 174
   LC-CDR3 having the amino acid sequence of SEQ ID NO: 189; or
(P1 G4) a VH region incorporating the following CDRs:
   HC-CDR1 having the amino acid sequence of SEQ ID NO: 106
   HC-CDR2 having the amino acid sequence of SEQ ID NO: 119
   HC-CDR3 having the amino acid sequence of SEQ ID NO: 223; and
a VL region incorporating the following CDRs:
   LC-CDR 1 having the amino acid sequence of SEQ ID NO: 151
   LC-CDR2 having the amino acid sequence of SEQ ID NO: 174
   LC-CDR3 having the amino acid sequence of SEQ ID NO: 258; or
(P1B12) a VH region incorporating the following CD Rs:
   HC-CDR1 having the amino acid sequence of SEQ ID NO: 106
   HC-CDR2 having the amino acid sequence of SEQ ID NO: 119
   HC-CDR3 having the amino acid sequence of SEQ ID NO: 224; and
a VL region incorporating the following CDRs:
   LC-CDR 1 having the amino acid sequence of SEQ ID NO: 235
   LC-CDR2 having the amino acid sequence of SEQ ID NO: 174
   LC-CDR3 having the amino acid sequence of SEQ ID NO: 189; or
(P1 C7) a VH region incorporating the following CDRs:
   HC-CDR1 having the amino acid sequence of SEQ ID NO: 106
   HC-CDR2 having the amino acid sequence of SEQ ID NO: 119
   HC-CDR3 having the amino acid sequence of SEQ ID NO: 225; and
a VL region incorporating the following CDRs:
   LC-CDR1 having the amino acid sequence of SEQ ID NO: 151
   LC-CDR2 having the amino acid sequence of SEQ ID NO: 245
   LC-CDR3 having the amino acid sequence of SEQ ID NO: 189.

In some embodiments the antigen-binding molecule comprises:
a VH region comprising an amino acid sequence having at least 85% sequence identity to one of SEQ ID NOS:71 to 84; and
a VL region comprising an amino acid sequence having at least 85% sequence identity to one of SEQ ID NOS:89 to 102.

In some embodiments the antigen-binding molecule comprises:
(P1A10) a VH region comprising an amino acid sequence having at least 85% sequence identity to SEQ ID NO: 71; and
a VL region comprising an amino acid sequence having at least 85% sequence identity to SEQ ID NO: 89; or
(P1 B6) a VH region comprising an amino acid sequence having at least 85% sequence identity to SEQ ID NO: 72; and
a VL region comprising an amino acid sequence having at least 85% sequence identity to SEQ ID NO: 90; or
(P1C10) a VH region comprising an amino acid sequence having at least 85% sequence identity to SEQ ID NO: 73; and
a VL region comprising an amino acid sequence having at least 85% sequence identity to SEQ ID NO: 91; or
(P107) a VH region comprising an amino acid sequence having at least 85% sequence identity to SEQ ID NO: 74; and
a VL region comprising an amino acid sequence having at least 85% sequence identity to SEQ ID NO: 92; or
(P1E8) a VH region comprising an amino acid sequence having at least 85% sequence identity to SEQ ID NO: 75; and
a VL region comprising an amino acid sequence having at least 85% sequence identity to SEQ ID NO: 93; or
(P2B2) a VH region comprising an amino acid sequence having at least 85% sequence identity to SEQ ID NO: 76; and
a VL region comprising an amino acid sequence having at least 85% sequence identity to SEQ ID NO: 94; or
(P2B7) a VH region comprising an amino acid sequence having at least 85% sequence identity to SEQ ID NO: 77; and
a VL region comprising an amino acid sequence having at least 85% sequence identity to SEQ ID NO: 95; or
(P2D11) a VH region comprising an amino acid sequence having at least 85% sequence identity to SEQ ID NO: 78; and
a VL region comprising an amino acid sequence having at least 85% sequence identity to SEQ ID NO: 96; or
(P2F10) a VH region comprising an amino acid sequence having at least 85% sequence identity to SEQ ID NO: 79; and
a VL region comprising an amino acid sequence having at least 85% sequence identity to SEQ ID NO: 97; or
(P2H4) a VH region comprising an amino acid sequence having at least 85% sequence identity to SEQ ID NO: 80; and
a VL region comprising an amino acid sequence having at least 85% sequence identity to SEQ ID NO: 98; or
(P2D3) a VH region comprising an amino acid sequence having at least 85% sequence identity to SEQ ID NO: 81; and
a VL region comprising an amino acid sequence having at least 85% sequence identity to SEQ ID NO: 99; or
(P1 G4) a VH region comprising an amino acid sequence having at least 85% sequence identity to SEQ ID NO: 82; and
a VL region comprising an amino acid sequence having at least 85% sequence identity to SEQ ID NO: 100; or
(P1B12) a VH region comprising an amino acid sequence having at least 85% sequence identity to SEQ ID NO: 83; and
a VL region comprising an amino acid sequence having at least 85% sequence identity to SEQ ID NO: 101; of
(P1C7) a VH region comprising an amino acid sequence having at least 85% sequence identity to SEQ ID NO: 84; and
a VL region comprising an amino acid sequence having at least 85% sequence identity to SEQ ID NO: 102.

The present invention also provides an antigen-binding molecule, optionally isolated, which is capable of birding to CD122, comprising (i) an antigen-binding molecule according to the present invention, and (ii) an antigen-binding molecule capable of binding to common γ chain (CD132).

The present invention also provides an antigen-binding molecule, optionally isolated, which is capable of binding to common γ chain (CD132), comprising (i) an antigen-binding molecule according to the present invention, and (ii) an antigen-binding molecule capable of binding to CD122.

The present invention also provides an antigen-binding molecule, optionally isolated, which is capable of binding to CD122 and common γ chain (CD132), comprising (i) an antigen-binding molecule according to the present invention, and (ii) an antigen-binding molecule according to the present invention.

In some embodiments the antigen-binding molecule further comprises a cell membrane anchor region.

In some embodiments, the antigen binding molecule is an IL-2 receptor agonist.

In some embodiments, the antigen binding molecule is capable of reducing expression of PD-1 by T cells.

The present invention also provides a chimeric antigen receptor (CAR) comprising an antigen-binding molecule according to the present invention.

The present invention also provides an in vitro complex, optionally isolated, comprising an antigen-binding molecule or CAR according to the present invention bound to CD122 and/or CD132.

The present invention also provides a nucleic acid, optionally isolated, encoding an antigen-binding molecule according to the present invention.

The present invention also provides an expression vector comprising a nucleic acid according to the present invention.

The present invention also provides a cell comprising an antigen-binding molecule, a nucleic acid or an expression vector according to the present invention.

The present invention also provides a method for producing an antigen-binding molecule according to the present invention, the method comprising culturing a cell comprising a nucleic acid or expression vector according to the present invention under conditions suitable for expression of the antigen-binding molecule from the nucleic acid or expression vector.

The present invention also provides a composition comprising an antigen-binding molecule, nucleic acid, expression vector or cell according to the present invention.

The present invention also provides an antigen-binding molecule, nucleic acid, expression vector, cell or composition according to the present invention for use in a method of medical treatment or prophylaxis.

The present invention also provides an antigen-binding molecule, nucleic acid, expression vector, cell or composition according to the present invention for use in a method of treatment of prevention of a T cell dysfunctional disorder, a cancer or an infectious disease.

The present invention also provides the use of an antigen-binding molecule, nucleic acid, expression vector, cell or composition according to the present invention in the manufacture of a medicament for use in a method of treatment of prevention of a T cell dysfunctional disorder, a cancer or an infectious disease.

The present invention also provides a method of treating or preventing a T cell dysfunctional disorder, a cancer or an infectious disease, comprising administering to a subject a therapeutically or prophylactically effective amount of an antigen-binding molecule, nucleic acid, expression vector, cell or composition according to the present invention.

In some embodiments the cancer is selected from the group consisting of: colon cancer, colon carcinoma, colorectal cancer, nasopharyngeal carcinoma, cervical carcinoma, oropharyngeal carcinoma, gastric carcinoma, hepatocellular carcinoma, head and neck cancer, head and neck squamous cell carcinoma (HNSCC), oral cancer, laryngeal cancer, prostate cancer, lung cancer, small cell lung cancer, non-small cell lung cancer, bladder cancer, urothelial carcinoma, melanoma, advanced melanoma, renal cell carcinoma, ovarian cancer or mesothelioma.

In some embodiments the antigen binding molecule is administered in combination with a therapeutically effective amount of an agent capable of inhibiting signalling mediated by an immune checkpoint protein. In some embodiments the immune checkpoint protein is PD-1. CTLA-4, LAG-3, TIM-3. VISTA, TIGIT or BTLA.

The present invention also provides a method for generating or expanding a population of immune cells, comprising contacting immune cells in vitro, in vivo or ex vivo with an antigen-binding molecule, nucleic acid, expression vector, cell or composition.

The present invention also provides a chimeric antigen receptor (CAR) comprising an antigen-binding molecule according to the present invention.

The present invention also provides an in vitro complex, optionally isolated, comprising an antigen-binding molecule or CAR according to the present invention bound to CD122 and/or CD132.

The present invention also provides a nucleic acid, optionally isolated, encoding e CAR according to the present invention.

The present invention also provides an expression vector comprising a nucleic acid according to the present invention.

The present invention also provides a cell comprising a CAR, a nucleic acid, or an expression vector according to the present invention.

The present invention also provides a composition comprising a CAR, a nucleic acid, an expression vector or a cell according to the present invention.

The present invention also provides a CAR, a nucleic acid, an expression vector, a cell or a composition according to the present invention for use in a method of medical treatment or prophylaxis.

### DESCRIPTION

Treatment with IL-2 is an approved immunotherapy for the treatment of cancer, and works by promoting proliferation and activity of effector immune cells such as T cells and NK calls (see e.g. Skorombolas and Frelinger. Expert Rev Clin Immunol. 2014; 10(2): 207-217).

However, there several drawbacks associated with IL-2 therapy. IL-2 has a very short half-life in serum, and so large doses and regular administration is required to achieve stimulation of T cell and NK cell proliferation/activity. This is problematic because high doses of IL-2 cause increases in levels of proinflammatory cytokines sometimes referred to as “cytokine storm”, which is thought to be a result of the widespread stimulation of immune cells. The cytokine storm is in turn thought to be responsible for many of the unwanted side effects of IL-2 treatment, including vascular leak syndrome (VLS). Furthermore, IL-2 is able to act on Tregs (which express the high-affinity IL-2Rα/β/γc receptors), and so treatment with IL-2 induces expansion of this suppressor T cell subset which can downregulate effector immune cell activity.

The inventors have designed and produced agonist antibodies which selectively bind to and activate intermediate-affinity IL-2Rβ/γc receptors. The antibodies are demonstrated to mimic the effect of IL-2 on cells expressing CD122 and CD132, causing expansion of effector immune cells. Unlike IL-2, the bispecific antibodies of the present invention preferentially stimulate proliferation of effector immune cells (which express intermediate-affinity IL-2Rβ/γc receptors) over regulatory T cells (which express high levels of the high-affinity IL-2Rα/β/γc receptors). Moreover, they have an increased serum half-life as compared to IL-2, and can therefore be administered less frequently and/or at a lower dose.

IL-2Rβ (CD122) and Common Gamma Chain (γc; CD132)

Human IL-2Rβ (also known as CD122, IL15RB and P70-75) is the protein identified by UniProt P14784-1, v1 (SEQ ID NO: 434). The N-terminal 26 amino acids of SEQ ID NO: 434 constitute a signal peptide, and so the mature for (i.e. after processing to remove the signal peptide) of human CD122 protein has the amino acid sequence shown in SEQ ID NO: 435. Amino acids 27 to 240 of SEQ ID NO: 434 constitute the extracellular domain of CD122, shown in SEQ ID NO: 436.

In this specification “IL-2Rβ” or “CD122” refers to CD122 from any species and includes isoforms, fragments, variants or homologues of CD122 from any species.

As used herein, a “fragment”, “variant” or “homologue” of a protein may optionally be characterised as having at least 60%, preferably one of 70%, 75%, 80%, 85%, 90%, 91%, 92%, 93%, 94%, 95%, 96%, 97%, 98%, 99% or 100% amino acid sequence identity to the amino acid sequence of the reference protein. Fragments, variants, isoforms and homologues of a reference protein may be may be characterised by ability to perform a function performed by the reference protein.

A “fragment” generally refers to a fraction of the reference protein. A “variant” generally refers to a protein having an amino acid sequence comprising one or more amino acid substitutions, insertions, deletions or other modifications relative to the amino acid sequence of the reference protein, but retaining a considerable degree of sequence identity (e.g. at least 60%) to the amino acid sequence of the reference protein. An “isoform” generally refers to a variant of the reference protein expressed by the same species as the species of the reference protein. A “homologue” generally refers to a variant of the reference protein produced by a different species as compared to the species of the reference protein. For example, human CD122 (P14784-1, v1; SEQ ID NO 434) and cynomolgus macaque CD122 (UniProt: Q38J85-1, v1) are homologues of one another.

A “fragment” of a reference protein may be of any length (by number of amino acids), although may optionally be at least 25% of the length of the reference protein (that is, the protein from which the fragment is derived) and may have a maximum length of one of 50%, 75%, 80%, 85%, 90%, 91%, 92%, 93%, 94%, 95%, 96%, 97%, 98%, or 99% of the length of the reference protein.

A fragment of CD122 may have a minimum length of one of 10, 20, 30, 40, 50, 100, 150, 200, 250 or 300 amino acids, and may have a maximum length of one of 20, 30, 40, 50, 100, 160, 200, 250, 300, 360, 400, 450 or 500 amino acids.

In some embodiments, the CD122 is mammalian CD122 (e.g. cynomolgous, human and/or rodent (e.g. rat and/or murine) CD122). Isoforms, fragments, variants or homologues of CD122 may optionally be characterised as having at least 70%, preferably one of 80%, 85%, 90%, 91%, 92%, 93%, 94%, 95%, 96%, 97%, 98%, 99% or 100% amino acid sequence identity to the amino acid sequence of immature or mature CD122 from a given species, e.g. human CD122. Isoforms, fragments, variants of homologues of CD122 may optionally be functional isoforms, fragments, variants or homologues, e.g. having a functional property/activity of the reference CD122 (e.g. full-length human CD122), as determined by analysis by a suitable assay for the functional property/activity. For example, an isoform, fragment, variant of homologue of CD122 may display one or more of association with one or more of CD132, IL-2Rα (CD25) or IL-15Rα (CD215), or binding to IL-2 or IL-15.

In some embodiments, the CD122 has at least 70%, preferably one of 80%, 85%, 80%, 91%, 82%, 93%, 94% 95%, 96% 97%, 98%, 99% or 100% amino acid sequence identity to one of SEQ ID NOs:434 to 436.

Human common gamma chain (γc; also known as CD132, IL-2Rβ and CIDX) is the protein identified by UniProt P31785-1, v1 (SEQ ID NO: 437). The N-terminal 23 amino acids of SEQ ID NO: 437 constitute a signal peptide, and so the mature form (i.e. after processing to remove the signal peptide) of human CD132 protein has the amino acid sequence shown in SEQ ID NO: 438. Amino acids 23 to 262 of SEO ID NO: 437 constitute the extracellular domain of CD132, shown in SEQ ID NO: 439.

In this specification “γc” or “CD132” refers to CD132 from any species and includes isoforms, fragments, variants of homologues of CD132 from any species.

In some embodiments, the CD132 is mammalian CD132 (e.g. cynomolgous, human and/or rodent (e.g. rat and/or murine) CD132). Isoforms, fragments, variants or homologues of CD132 may optionally be characterised as having at least 70%, preferably one of 80%, 85%, 90%, 91%, 92%, 93%, 94%, 95%, 96%, 97%, 98%, 99% or 100% amino acid sequence identity to the amino acid sequence of immature or mature CD132 from a given species, e.g. human CD132. Isoforms, fragments, variants or homologues of CD132 may optionally be functional isoforms, fragments, variants or homologues, e.g. having a functional property/activity of the reference CD132 (e.g. full-length human CD132), as determined by analysis by a suitable assay for the functional property/activity. For example, an isoform, fragment, variant or homologue of CD132 may display one or more of association with one or more of CD122, IL-2Rα, L-15Rα, IL-4R (CD124), IL-9R (CD129), IL-21R (CD360) or IL7R (CD127), or binding to one or more of IL-2, IL-15, IL-4, IL-9, IL-21 or IL-7.

A fragment of CD132 may have a minimum length of one of 10, 20, 30, 40, 50, 100, 150, 200, 250 or 300 amino acids, and may have a maximum length of one of 20, 30, 40, 50, 100, 150, 200, 250, 300, 350 amino acids.

In some embodiments, the CD132 has at least 70%, preferably one of 80%, 85%, 90%, 91%, 92%, 93%, 94% 95%, 96%, 97%, 98%, 99% or 100% amino acid sequence identity to one of SEQ ID NOs:438 to 440.

IL-2 receptors and their biology is described, for example, Skrombolas and Frelinger, Expert Rev Clin Immunol. (2014)10(2): 207-217, which is hereby incorporated by reference in its entirety.

CD122 and CD132 participate in the formation of receptors for IL-2, CD122 and CD132 associate with IL-2Ro (CD25) to form the trimeric, high-affinity IL-2 receptor (sometimes designated “IL-2Rα/β/γc” or “CD26/CD122/CD132”), which binds to IL-2 with a Kd of ˜10 pM. CD122 and CD132 are also capable of associating to form a functional intermediate-affinity IL-2 receptor (sometimes designated “IL-2Rβ/γc” or “CD122/CD132”), which binds to IL-2 with a Kd of ˜1 nM.

The composition of the receptors, the number, and likely signalling capacity can vary with the cell type and activation stage. IL-2 receptors are expressed at relatively low levels on resting naive T cells. However, activated CD4 and CD8 T cells begin to express high levels of CD25, which allows them to bind IL-2 efficiently. CD25 is expressed at higher amounts (8-10 fold) compared to CD122 and CD132, CD25 is thought to bind IL-2 initially, effectively increasing its concentration at the cell surface and inducing a conformational change in IL-2 which then subsequently binds to the CD122 and CD132 (Liao et al., Immunity (2013) 38(1): 13-25). NK cells and memory phenotype CD8 cells express high levels of CD122 and CD132 compared to naive cells and some NK cells can also express CD25 after stimulation with IL-2.

Importantly, CD4 regulatory T cells (Tregs) constitutively express high levels of CD25. Tregs act in multiple ways to down regulate many immune responses, including anti-tumor responses (see e.g. Shevach, Immunity (2009) 30(5):636-45)

Antigen-Binding Molecules

The present invention provides antigen-binding molecules. In aspects of the present invention the antigen-binding molecules are capable of binding to CD122. In aspects of the present invention the antigen-binding molecules are capable of binding to CD132. In aspects of the present invention the antigen-binding molecules are capable of binding to CD122 and CD132, in aspects of the present invention the antigen-binding molecules are capable of binding to CD122 and CD132, and comprise an antigen-binding molecule capable of binding to CD122 and an antigen-binding molecule capable of binding to CD132.

An “antigen-binding molecule” as used herein refers to a polypeptide or polypeptide complex which is capable of binding to a target antigen or antigens, and encompasses monoclonal antibodies, polyclonal antibodies, monospecific and multispecific antibodies (e.g., bispecific antibodies), and antibody fragments, as long as they display binding to the relevant target antigen(s).

The antigen-binding molecule of the present invention comprises a moiety or moieties capable of binding to the target antigen(s). The moiety capable of binding to a target antigen comprises an antibody heavy chain variable region (VH) and an antibody light chain variable region (VL) of an antibody capable of specific binding to the target antigen. In some embodiments, the moiety capable of binding to a target antigen comprises or consists of an aptamer capable of binding to the target antigen, e.g. a nucleic acid aptamer (reviewed, for example, in Zhou and Rossi Nat Rev Drug Discov. 2017 16(3): 181-202). In some embodiments, the moisty capable of binding to a target antigen comprises or consists of a antigen-binding peptide/polypeptide, e.g. a peptide aptamer, thioredoxin, monobody, anticalin, Kunitz domain, avimer, knottin, fynomer, atrimer, DARPin, affibody, nanobody (i.e. a single-domain antibody (sdAb)) affilin, armadillo repeat protein (ArmRP), OBody or fibronectin—reviewed e.g. in Reverdalto et al., Curr Top Med Chem. 2015; 15(12) 1082-1101, which is hereby incorporated by reference in its entirety (see also e.g. Boersma et al., J Biol Chem (2011) 286:41273-85 and Emanuel et al., Mabs (2011) 3:38-48).

The antigen-binding molecules of the present invention generally comprise antigen-binding moities comprising a VH and a VL of an antibody capable of specific binding to the target antigen. The antigen-binding moiety formed by a VH and a VL may also be referred to herein as an Fv region.

An antigen-binding molecule may be, or may comprise, an antigen-binding polypeptide, or an antigen-binding polypeptide complex. An antigen-binding molecule may comprise more than one polypeptide which together form an antigen-binding domain. The polypeptides may associate covalently or non-covalently. In some embodiments the polypeptides form part of a larger polypeptide comprising the polypeptides (e.g. in the case of scFv comprising VH and VL, or in the case of scFab comprising VH-CH1 and VL-CL).

An antigen-binding molecule may comprise or consist of one or more polypeptides. In some embodiments an antigen-binding molecule comprises 1, 2, 3, 4, 5, 6, 7, 8, 9, 10, 11 or 12 polypeptides. In some embodiments an antigen-binding molecule is a covalent or non-covalent complex of more than one polypeptide (e.g. 2, 3, 4, 6, 8, 10 or more polypeptides). For example, in some embodiments an antigen-binding molecule comprises two heavy chain polypeptides and two light chain polypeptides.

The antigen-binding molecules described herein preferably display specific binding to the relevant target (e.g. CD122 and/or CD132). As used herein, “specific binding” refers to binding which is selective for the antigen, and which can be discriminated from non-specific binding to non-target antigen. An antigen-binding molecule that specifically binds to a target molecule preferably binds the target with greater affinity, and/or with greater duration than it binds to other, non-target molecules.

An antigen-binding molecule described herein may be capable of binding to CD122 as described herein. An antigen-binding molecule described herein may be capable of binding to CD132 as described herein. An antigen-binding molecule described herein may be capable of binding to CD122 as described herein and CD132 as described herein.

The ability of a given polypeptide to bind specifically to a given molecule can be determined by analysis according to methods known in the art, such as by ELISA. Surface Plasmon Resonance (SPR; see e.g. Hearty et al., Methods Mol Biol (2012) 907:411-442). Bio-Layer Interferometry (see e.g. Lad et al. (2015) J Biomol Screen 20(4): 498-507), flow cytometry, or by a radiolabeled antigen-binding assay (RIA) enzyme-linked immunosorbent assay. Through such analysis binding to a given molecule can be measured and quantified. In some embodiments, the binding may be the response detected in a given assay.

In some embodiments, the extent of binding of the antigen-binding molecule to an non-target molecule is less than about 10% of the binding of the antibody to the target molecule as measured, e.g. by ELISA, SPR, Bio-Layer Interferometry or by RIA. Alternatively, binding specificity may be reflected in terms of binding affinity where the antigen-binding molecule binds with a dissociation constant (KD) that is at least 0.1 order of magnitude (i.e. 0.1×10ⁿ, where n is an integer representing the order of magnitude) greater than the KD of the antigen-binding molecule towards a non-target molecule. This may optionally be one of at least 0.2, 0.3, 0.4, 0.5, 0.6, 0.7, 0.8, 0.9, 1.0, 1.5, or 2.0.

In certain embodiments, the antigen-binding molecule binds to the target molecule with a KD of ≤10 μM, ≤1 μM, ≤100 nM, ≤10 nM, ≤1 nM, ≤0.1 nM, ≤0.01 nM, or ≤0.001 nM as determined by analysis according to SPR, Bio-Layer Interferometry or by RIA.

In some embodiments, the antigen-binding molecule binds to the same or an overlapping epitope of the target molecule as a reference antigen-binding molecule which is capable of binding to the target molecule (i.e. CD122 or CD132). In some embodiments, the antigen-binding molecule displays competitive binding with a reference antigen-binding molecule which is capable of binding to the target molecule. Whether a given antigen-binding molecule displays such competitive binding can be determined by various methods known to the skilled person, including competition ELISA.

In some embodiments, the antigen-binding molecule comprises the complementarity-determining regions (CDRs) of an antigen-binding molecule which is capable of binding to the target molecule (i.e. CD122 of CD132). Antibodies generally comprise six CDRs; three in the light chain variable region (VL): LC-CDR1. LC-CDR2, LC-CDR3, and three in the heavy chain variable region (VH): HC-CDR1, HC-CDR2 and HC-CDR3. The six CDRs together define the paratope of the antibody, which is the part of the antibody which binds to the target molecule. There are several different conventions for defining antibody CDRs, such as those described in Kabat at al., Sequences of Proteins of Immunological Interest, 5th Ed. Public Health Service. National Institutes of Health, Bethesda, MD (1991). Chothia et al., J. Mol. Biol. 196:901-917 (1987) and VBASE2, as described in Retter et al., Nucl. Acids Res. (2005) 33 (suppl 1); D671-D674. Unless otherwise specified, CDRs of the antigen-binding molecules described herein are defined according to Kabat et al., Sequences of Proteins of Immunological Interest, 5th Ed. Public Health Service. National Institutes of Health, Bethesda. MO (1991).

The antigen-binding molecule may be designed and prepared using the sequences of monoclonal antibodies (mAbs) capable of binding to CD122, and mAbs capable binding to CD132 described herein. Antigen-binding regions of antibodies, such as single chain variable fragment (scFv), Fab and Fab₂ fragments may also be used/provided. An ‘antigen-binding region’ is any fragment of an antibody which is capable of binding to the target for which the given antibody is specific.

In some embodiments, the antigen-binding molecule of the present invention is a CD122-binding molecule. In some embodiments, the antigen-binding molecule comprises or consists of a CD122-binding molecule. In some embodiments the antigen-binding molecule comprises a heavy chain variable (VH) region comprising HC-CDR1, HC-CDR2 and HC-CDR3 of a CD122-binding antibody clone described herein, or a variant thereof in which one or two or three amino acids in one or more of HC-CDR1, HC-CDR2, HC-CDR3 are substituted with another amino acid. In some embodiments the antigen-binding molecule comprises a light chain variable (VL) region comprising LC-CDR1, LC-CDR2 and LC-CDR3 of a CD122-binding antibody clone described herein, or a variant thereof in which one or two or three amino acids in one or more of LC-CDR1, LC-CDR2, LC-CDR3 are substituted with another amino acid. In some embodiments the antigen-binding molecule comprises a VH region comprising HC-CDR1, HC-CDR2 and HC-CDR3 and a VL region comprising LC-CDR1, LC-CDR2 and LC-CDR3 of a CD122-binding antibody clone described herein, or a variant thereof in which one or two or three amino acids in one or more of HC-CDR1, HC-CDR2, HC-CDR3, LC-CDR1, LC-CDR2 or LC-CDR3 are substituted with another amino acid.

In some embodiments the antigen-binding molecule comprises a VH region which comprises or consists of an amino acid sequence having at least 70%, more preferably one of at least 75%, 80%, 85%, 86%, 87%, 88%, 89%, 90%, 91%, 92%, 93%, 94%, 95%, 96%, 97%, 98%, 99%, or 100%, sequence identity to VH region of a CD122-binding antibody clone described herein. In some embodiments the antigen-binding molecule comprises a VL region which comprises or consists of an amino acid sequence having at least 70%, more preferably one of at least 75%, 80%, 85%, 86%, 87%, 88%, 89%, 90%, 91%, 92%, 93% 94%, 95% 96%, 97%, 98%, 99%, or 100%, sequence identity to VL region of a CD122-binding antibody clone described herein. In some embodiments the antigen-binding molecule comprises a VH region which comprises or consists of an amino acid sequence having at least 70%, more preferably one of at least 75%, 80%, 85%, 86%, 87%, 88%, 89%, 90%, 91%, 92%, 93%, 94%, 95%, 96%, 37%, 98%, 99%, or 100%, sequence identity to VH region of a CD122-binding antibody clone described herein and a VL region which comprises or consists of an amino acid sequence having at least 70%, more preferably one of at least 75%, 80%, 85%, 86%, 87%, 88%, 89%, 90%, 91%, 92%, 93%, 94%, 95%, 96%, 97%, 98%, 99%, or 100%, sequence identity to VL region of a CD122-binding antibody clone described herein.

In some embodiments a CD122-binding antibody clone is selected from: P2C4, P2C4_A4, P2C4_B1, P2C4_B5, P2C4_C1, P2C4_C4, P2C4_C7, P2C4_D10, P2C4_E6, P2C4_E7, P2C4_F8, P2C4_C1D10, P2C4_FW2, P2H7, P2D12, P1G11, P2C4_A9, P2C4_86, P2C4_E9, P2C4_B8, P2C4_B12, P2C4_C12. P2C4_E2, P2C4_E3, P2C4_E8, P2C4_F11, P2C4_G2, P2C4_G11, P2C4_H1, P2C4_H2, P2C4_H3, P1E7, P1B10. PIF3, P1D10, P1E1, P2B11, P2C9, P2C10, P2C11, P2E6, P2E11, P2F9 and P2F10. In some embodiments a CD122-binding antibody clone is selected from: P1E7, P1B10, P1F3, P1D10, P1E1, P2811, P2C9, P2C10, P2C11, P2E6, P2E11, P2F9 and P2F10. In some embodiments the CD122-binding antibody clone is P2C4, P2C4_FW2, P2E6, P1D10, P1E7 or P1G11. In some embodiments the CD122-binding antibody clone is P2C4 or P2C4_FW2.

In some embodiments, the antigen-binding molecule of the present invention is a CD132-binding molecule. In some embodiments, the antigen-binding molecule comprises or consists of a CD132-binding molecule. In some embodiments the antigen-binding molecule comprises a heavy chain variable (VH) region comprising HC-CDR1, HC-CDR2 and HC-CDR3 of a CD132-binding antibody clone described herein, or a variant thereof in which one or two or three amino acids in one or more of HC-CDR1, HC-CDR2, HC-CDR3 are substituted with another amino acid. In some embodiments the antigen-binding molecule comprises a light chain variable (VL) region comprising LC-CDR1. LC-CDR2 and LC-CDR3 of a CD132-binding antibody clone described herein, or a variant thereof in which one or two or three amino acids in one or more of LC-CDR1, LC-CDR2, LC-CDR3 are substituted with another amino acid. In some embodiments the antigen-binding molecule comprises a VH region comprising HC-CDR1, HC-CDR2 and HC-CDR3 and a VL region comprising LC-CDR1. LC-CDR2 and LC-CDR3 of a CD132-binding antibody clone described herein, or a variant thereof in which one or two or three amino acids in one or more of HC-CDR1. HC-CDR2. HC-CDR3, LC-CDR1, LC-CDR2 or LC-CDR3 are substituted with another amino acid.

In some embodiments the antigen-binding molecule comprises a VH region which comprises or consists of an amino acid sequence having at least 70%, more preferably one of at least 75%, 80%, 85%, 86%, 87%, 88%, 89%, 90%, 91%, 92%, 93%, 94%, 95%, 96%, 97%, 98%, 99%, or 100%, sequence identity to VH region of a CD132-binding antibody clone described herein. In some embodiments the antigen-binding molecule comprises a VL region which comprises or consists of an amino acid sequence having at least 70%, more preferably one of at least 75%, 80%, 85%, 86%, 87%, 88%, 89%, 90%, 91%, 92%, 93%, 94%, 95%, 96%, 97%, 98%, 99%, or 100%, sequence identity to VL region of a CD132-binding antibody clone described herein. In some embodiments the antigen-binding molecule comprises a VH region which comprises or consists of an amino acid sequence having at least 70%, more preferably one of at least 75%, 80%, 85%, 86%, 87%, 88%, 89%, 90%, 91%, 92%, 93%, 94%, 95%, 96%, 97%, 98%, 99% or 100%, sequence identity to VH region of a CD132-binding antibody done described herein and a VL region which comprises or consists of an amino acid sequence having at least 70%, more preferably one of at least 75%, 80%, 85%, 86%, 87%, 88%, 89%, 90%, 91%, 92%, 93%, 94%, 95%, 96%, 97%, 98%, 99%, or 100%, sequence identity to VL region of a CD132-binding antibody clone described herein.

In some embodiments a CD132-binding antibody clone is selected from: P1A3, P1A3_B3, P1A3 E8, P1A3_E9, P2B9, P1A3 B4, P1A3 FW2, P1A10, P186, P1C10, P1D7, P1E8, P282, P287, P2D11. P2F10, P2H4, P2D3, P1G4, P1B12 and P1C7. In some embodiments a CD132-binding antibody clone is selected from: P1A10, P186, P1C10, P1D7, P1E8, P2B2, P2B7, P2D11, P2F10, P2H4, P2D3, P1G4, P1B12 and P1C7. In some embodiments the CD132-binding antibody clone is P1A10. In some embodiments the CD132-binding antibody clone is P1A3 or P1A3_FW2.

In some embodiments the antigen-binding molecule of the present invention comprises:
a VH region comprising HC-CDR1, HC-CDR2 and HC-CDR3 and a VL region comprising LC-CDR1, LC-CDR2 and LC-CDR3 of a CD122-binding antibody clone, or a variant thereof in which one or two or three amino acids in one or more of HC-CDR1. HC-CDR2, HC-CDR3. LC-CDR1. LC-CDR2 or LC-CDR3 are substituted with another amino acid; and
a VH region comprising HC-CDR1, HC-CDR2 and HC-CDR3 and a VL region comprising LG-CDR1, LC-CDR2 and LC-CDR3 of a CD132-binding antibody clone, or a variant thereof in which one or two or three amino acids in one or more of HC-CDR1, HC-CDR2, HC-CDR3. LC-CDR1. LC-CDR2 or LC-CDR3 are substituted with another amino acid;
wherein:
the CD122-binding antibody clone is P204 and the CD132-binding antibody clone is P1A10; or
the CD122-binding antibody clone is P2C4 and the CD132-binding antibody clone is P1A3; or
the CD122-binding antibody clone is P2C4_FW2 and the CD132-binding antibody clone is P1A3;
the CD122-binding antibody clone is P2E6 and the CD132-binding antibody clone is P1A10; or
the CD122-binding antibody clone is P1D10 and the CD132-binding antibody clone is PIA 10; or
the CD122-binding antibody clone is P1E7 and the CD132-binding antibody clone is P1A10; or
the CD122-binding antibody clone is P1G11 and the CD132-binding antibody clone is P1A10.

In some embodiments the antigen-binding molecule of the present invention comprises:
a VH region having at least 70% sequence identity to the VH region of a CD122-binding antibody clone, and a VL region having at least 70% sequence identity to the VL region of the CD122-binding antibody clone; and
a VH region having at least 70% sequence identity to the VH region of a CD132-binding antibody clone, and a VL region having at least 70% sequence identity to the VL region of the CD132-binding antibody clone;
wherein:
the CD122-binding antibody clone is P204 and the CD132-binding antibody clone is P1A10; or
the CD122-binding antibody clone is P204 and the CD132-binding antibody clone is P1A3; or
the CD122-binding antibody clone is P204_ FW2 and the CD132-binding antibody clone is P1A3;
the CD122-binding antibody clone is P2E6 and the CD132-binding antibody clone is P1A10; or
the CD122-binding antibody clone is P1D10 and the CD132-binding antibody clone is P1A10; or
the CD122-binding antibody clone is P1E7 and the CD132-binding antibody clone is P1A10; or
the CD122-binding antibody clone is P1G11 and the CD132-binding antibody clone is P1A10.

In some embodiments the antigen-binding molecule of the present invention composes:
(i) a CD122-binding antigen-binding molecule comprising:
   (P204) a VH region incorporating the following CDRs:
      HC-CDR1 having the amino acid sequence of SEQ ID NO: 103
      HC-CDR2 having the amino acid sequence of SEQ ID NO: 116
      HC-CDR3 having the amino acid sequence of SEQ ID NO: 128; and
   a VL region incorporating the following CDRs:
      LC-CDR1 having the amino acid sequence of SEQ ID NO: 145
      LC-CDR2 having the amino acid sequence of SEQ ID NO: 162
      LC-CDR3 having the amino acid sequence of SEQ ID NO: 177; and
(ii) a CD132-binding antigen-binding molecule comprising:
   (P1A10) a VH region incorporating the following CDRs:
      HC-CDR1 having the amino acid sequence of SEQ ID NO: 196
      HC-CDR2 having the amino acid sequence of SEQ ID NO 204
      HC-CDR3 having the amino acid sequence of SEQ ID NO: 212; and
   a VL region incorporating the following CDRs:
      LC-CDR1 having the amino acid sequence of SEQ ID NO: 227
      LC-CDR2 having the amino acid sequence of SEQ ID NO: 238
      LC-CDR3 having the amino acid sequence of SEQ ID NO: 248.

In some embodiments the antigen-binding molecule comprises:
(1) e CD122-binding antigen-binding molecule comprising:
   (P2C4) a VH region comprising an amino acid sequence having al least 85% sequence identity to SEQ ID NO: 1; and
   a VL region comprising an amino acid sequence having at least 85% sequence identity to SEQ ID NO: 35; and
(ii) & CD132-binding antigen-binding molecule comprising:
   (P1A10) a VH region comprising an amino acid sequence having at least 65% sequence identity to SEQ ID NO: 71; and
   a VL region comprising an amino acid sequence having at least 85% sequence identity to SEQ ID NO: 89.

In some embodiments, the antigen-binding molecule may comprise a variant of a reference VL/VH region. e.g. comprising 1, 2, 3, 4, 5, 6, 7, 8, 9 or 10 substitutions with respect to the amino acid sequence of the reference VL/VH region(s) In some embodiments the substitution(s) are not in the GDRs. In some embodiments the substitution(s) are in the framework region(s)—i.e, the amino acid sequences of the VL/VH region(s) other than the CDRs.

In some embodiments, the substitutions are conservative substitutions, for example according to the following Table. In some embodiments, amino acids in the same block in the middle column are substituted. In some embodiments, amino acids in the same line in the rightmost column are substituted:

| | | |
|---|---|---|
| ALIPHATIC | Non-polar | G A P |
| | | I L V |
| | Polar-uncharged | C S T M |
| | | N Q |
| | Polar-charged | D E |
| | | K R |
| AROMATIC | | H F W Y |

In some embodiments the antigen-binding molecule of the present invention does not comprise a combination of CDRs or VL/VH domains disclosed in WO 2017/021540 A1 (hereby incorporated by reference in its entirety).

In some embodiments the CD122-binding antigen-binding molecule according to the invention comprises or consists of an amino acid sequence having at least 70%, more preferably one of at least 75%, 80%, 85%, 86%, 87%, 88%, 89%, 90%, 91%, 92%, 93%, 94%, 95%, 96%, 97%, 98%, 99%, or 100%, sequence identity to one of SEQ ID NOs:265 to 308. In some embodiments the CD122-binding antigen-binding molecule according to the invention comprises or consists of an amino acid sequence having at least 70%, more preferably one of at least 75%, 80%, 85%, 86%, 87%, 88%, 89%, 90%, 91%, 92%, 93%, 94%, 95%, 96%, 97%, 98%, 99%, or 100%, sequence identity to one of SEQ ID NOs:296 to 308.

In some embodiments the CD132-binding antigen-binding molecule according to the invention comprises or consists of an amino acid sequence having at least 70%, more preferably one of at least 75%, 80%, 85%, 86% 87%, 88%, 89%, 90%, 91%, 92%, 93%, 94%, 95%, 96%, 97%, 98%, 99% or 100%, sequence identity to one of SEQ ID NOs:309 to 329. In some embodiments the CD132-binding antigen-binding molecule according to the invention comprises or consist of an amino acid sequence having at least 70%, more preferably one of at least 75%, 80%, 85% 86%, 87%, 88%, 89%, 90%, 91%, 92%, 93%, 94%, 95%, 96%, 97%, 98%, 99%, or 100%, sequence identity to one of SEQ ID NOs:316 to 329.

In some embodiments, the CD122-binding antigen-binding molecule according to the present invention lacks HC-CDR1, HG-CDR2, HC-CDR3, LC-CDR1, LC-CDR2, and LC-CDR3 of one or more of the following clones: P2C4, P2C4_A4, P2C4_B1, P2C4_B5, P2C4_C1, P2C4_C4, P2C4_C7, P2C4_D10, P2C4_E6, P2C4_E7, P2C4 FB, P2C4_C1D10, P2C4_FW2, P2H7, P2012, P1G11, P2C4_A9, P2C4_B6. P2C4_E9, P2C4_B8, P2C4_B12, P2C4_C12, P2C4_E2, P2C4_E3, P2C4_E8, P2C4_F11, P2C4 G2, P2C4_G11, P2C4_H1, P2C4_H2 and P2C4_H3. In some embodiments the CD122-binding antigen-binding molecule according to the present invention lacks the VL domain sequence and/or the VH domain sequence of one or more of said clones. In some embodiments the CD122-binding antigen-binding molecule according to the present invention lacks the VL domain sequence and/or the VH domain sequence of one or more of said clones.

In some embodiments, the CD132-binding antigen-binding molecule according to the present invention lacks HC-CDR1, HC-CDR2. HC-CDR3, LC-CDR1, LC-CDR2, and LC-CDR3 of one or more of the following clones: P1A3, P1A3_B3, P1A3_E8, P1A3_E9, P1A3B4, P1A3 FW2 and P289 In some embodiments the CD132-binding antigen-binding molecule according to the present invention lacks the VL domain sequence and/or the VH domain sequence of one or more of said clones.

Antigen-binding molecules may be produced by a process of affinity maturation in which a modified antibody is generated that has an improvement in the affinity of the antibody for antigen, compared to an unmodified parent antibody. Affinity-matured antigen-binding molecules may be produced by procedures known in the art, e.g., Marks et al., Bio/Technology 10:779-783 (1992); Barbas et al. Proc Nat. Acad. Sci. USA 91:3809-3813 (1994); Schier et al. Gene 169:147-155 (1995); Yelton et al. J. Immunol. 155.1994-2004 (1996); Jackson et al., J. Immunol. 154(7):331 0-15 9 (1995); and Hawkins el al, J. Mol. Biol. 226:869-896 (1992).

The VL and VH region of an antigen-binding region of an antibody together constitute the Fv region. In some embodiments, the antigen-binding molecule according to the present invention comprises, of consists of, an Fv region which binds to CD122. In some embodiments, the antigen-binding molecule comprises, or consists of, an Fv region which binds to CD132.

The VL and light chain constant (CL) region, and the VH region and heavy chain constant 1 (CH1) region of an antigen-binding region of an antibody together constitute the Fab region. In some embodiments, the antigen-binding molecule of the antigen-binding molecule described herein comprises, or consists of, a Fab region which binds to CD122. In some embodiments, the antigen-binding molecule comprises, or consists of, a Fab region which birds to CD132.

In some embodiments, the antigen-binding molecule described herein comprises, or consists of, a whole antibody which binds to CD122. In some embodiments, the antigen-binding molecule described herein comprises, or consists of, a whole antibody which binds to a CD132. As used herein, “whole antibody” refers to an antibody having a structure which is substantially similar to the structure of an immunoglobulin (Ig). Different kinds of immunoglobulins and their structures are described e.g. in Schroeder and Cavacini J Allergy Clin Immunol. (2010) 125(202); S41-S52, which is hereby incorporated by reference in its entirety.

Immunoglobulins of type G (i.e. IgG) are −150 kDa glycoproteins comprising two heavy chains and two light chains. From N- to C-terminus, the heavy chains comprise a VH followed by a heavy chain constant region comprising three constant domains (CH1, CH2, and CH3), and similarly the light chains comprise #VL followed by e CL. Depending on the heavy chain, immunoglobulins may be classed as IgG (e.g. IgG1, IgG2, IgG3, IgG4), IgA (e.g. IgA1, IgA2), IgD, IgE, or IgM. The light chain may be kappa (κ) or lambda (λ).

In some embodiments the immunoglobulin heavy chain constant sequence is human immunoglobulin G 1 constant (IGHG1; UniProt: P01857-1, v1; SEQ ID NO. 440). Positions 1 to 98 of SEQ ID NO: 440 form the CH1 region (SEQ ID NO: 441). Positions 99 to 110 of SEQ ID NO: 440 form a hinge region between CH1 and CH2 regions (SEQ ID NO: 442). Positions 111 to 223 of SEQ ID NO: 440 form the CH2 region (SEQ ID NO: 443). Positions 224 to 330 of SEQ ID NO: 440 form the CH3 region (SEQ ID NO: 444).

In some embodiments a CH1 region comprises or consists of the sequence of SEQ ID NO: 441, or a sequence having at least 60%, preferably one of 70%, 75%, 80%, 86%, 90%, 91%, 92%, 93%, 94%, 95%, 96%, 97%, 98%, 99% or 100% amino acid sequence identity to the amino acid sequence of SEQ ID NO: 441. In some embodiments a CH1-CH2 hinge region comprises or consists of the sequence of SEQ ID NO: 442, or a sequence having at least 60%, preferably one of 70%, 75%, 80%, 85%, 90%, 91%, 92%, 93%, 94%, 95%, 96%, 97%, 98%, 99% or 100% amino acid sequence identity to the amino acid sequence of SEQ ID NO: 442. In some embodiments a CH2 region comprises or consists of the sequence of SEQ ID NO: 443, or a sequence having at least 60%, preferably one of 70%, 75%, 80%, 85%, 90%, 91%, 92%, 93%, 94%, 95%, 96%, 97%, 98%, 99% or 100% amino acid sequence identity to the amino acid sequence of SEQ ID NO: 443. In some embodiments a CH3 region comprises or consists of the sequence of SEQ ID NO: 444, or a sequence having at least 60%, preferably one of 70%, 75%, 80%, 85%, 90%, 91%, 92%, 93%, 94%, 95%, 96%, 97%, 98%, 99% or 100% amino acid sequence identity to the amino acid sequence of SEQ ID NO: 444.

It will be appreciated that CH3 regions may be provided with further substitutions in accordance with modification to an Fc region of the antigen-binding molecule as described herein. In some embodiments a CH3 region comprises or consists of the sequence of SEQ ID NO: 447, or a sequence having at least 60%, preferably one of 70%, 75%, 80%, 85%, 90%, 91%, 92%, 93%, 94%, 95%, 96%, 97%, 98%, 99% or 100% amino acid sequence identity to the amino acid sequence of SEQ ID NO: 447. In some embodiments a CH3 region comprises or consists of the sequence of SEQ ID NO: 448, or a sequence having at least 60%, preferably one of 70% 75%, 80%, 85%, 90%, 91%, 92%, 93%, 94%, 95%, 96%, 97%, 98%, 99% or 100% amino acid sequence identity to the amino acid sequence of SEQ ID NO: 448.

In some embodiments the antigen-binding molecule of the present invention comprises one or more regions of an immunoglobulin light chain constant sequence. In some embodiments the immunoglobulin light chain constant sequence is human immunoglobulin kappa constant (IGKC; C_{K}; UniProt: P01834-1, v2; SEQ ID NO: 445). In some embodiments the immunoglobulin light chain constant sequence is a human immunoglobulin lambda constant (IGLC: Cλ), e.g. IGLC1, IGLC2, IGLC3, IGLC6 or IGLC7. In some embodiments a CL region comprises or consists of the sequence of SEQ ID NO: 445, or a sequence having at least 80%, preferably one of 70%, 75%, 80%, 85%, 90%, 91%, 92%, 93%, 94%, 95%, 96%, 97%, 98%, 99% or 100% amino acid sequence identity to the amino acid sequence of SEQ ID NO: 445.

In some embodiments, the antigen-binding molecule described herein comprises, or consists of, an IgG (e.g. IgG1, IgG2, IgG3, IgG4), IgA (e.g. IgA1, IgA2), IgD, IgE, or IgM which binds to CD122 In some embodiments, the antigen-binding molecule described herein comprises, or consists of, an IgG (e.g. IgG1, IgG2, IgG3, IgG4), IgA (e.g. IgA1, IgA2), IgD, IgE, or IgM which binds to CD132.

The antigen-binding molecules according to the present invention may be provided in any suitable format.

Aspects of the present invention relate to multispecific antigen-binding molecules. By “multispecific” it is meant that the antigen-binding molecule displays specific binding to more than one target. In particular, the antigen-binding molecule is binding to CD122 and CD132, and so is at least bispecific. The term “bispecific” means that the antigen-binding molecule is able to bind specifically to at least two distinct antigenic determinants.

Multispecific antigen-binding molecules described herein display at least monovalent binding with respect to CD122, and also displays at least monovalent binding with respect to CD132. Binding valency refers to the number of binding sites in an antigen-binding molecule for a given antigenic determinant. For example, bispecific antigen-binding molecules in scFv-KiH-Fc, CrossMab and Duobody formats are provided herein, which are monovalent with respect to binding to CD122, and monovalent with respect to binding to CD132.

In some embodiments the antigen-binding molecule comprises one binding site for CD122, and one binding site for CD132. In some embodiments the antigen-binding molecule comprises more than one binding site (e.g. two, three) for CD122. In some embodiments the antigen-binding molecule comprises more than one binding site (e.g. two, three) for CD132. In some embodiments the antigen-binding molecule comprises more than one binding site (e.g. two, three) for CD122, and more than one binding site (e.g. two, three) for CD132.

In some embodiments the antigen-binding molecule is multivalent (e.g. bivalent, trivalent) for CD122. In some embodiments the antigen-binding molecule is multivalent (e.g. bivalent, trivalent) for CD132. In some embodiments the antigen-binding molecule is multivalent (e.g. bivalent, trivalent) for CD122, and multivalent (e.g. bivalent, trivalent) for CD132.

In some embodiments the antigen-binding molecule comprises two binding sites for CD122. In some embodiments the antigen-binding molecule comprises two binding sites for CD132. In some embodiments the antigen-binding molecule comprises two binding sites for CD122, and two binding sites for CD132.

Multispecific antigen-binding molecules according to the invention may be provided in any suitable format, such as those formats described in Kontermann MAbs 2012, 4(2), 182-197, which is hereby incorporated by reference in its entirety. For example, an antigen-binding molecule may be a bispecific antibody conjugate (e.g. an IgG2. F(ab′)₂ of CovX-Body), a bispecific IgG or IgG-like molecule (e.g. AN IGG, SCFV₄-Ig, IgG-scFv, scFv-IgG, DVD-Ig, IgG-sVD, sVD-IgG, 2 in 1-IgG, mAB², or Tandemab common LC), an asymmetric bispecific IgG or IgG-like molecule (e.g. a kih IgG, kih IgG common LC, CrossMab, kih IgG-scFab, mAb-Fv, charge pair or SEED-body), a small bispecific antibody molecule (e.g. a Diabody (db), dsDb, DART, scDb, tandAbs, tandem scFv (taFv), tandem dAb/VHH, triple body, triple head, Fab-scFv, of F(ab′)₂-scFv₂), a bispecific Fc and C_{H}3 fusion protein (e.g. a taFv-Fc, Di-diabody, scDb-C_{H}3, scFv-Fc-scFv, HCAb-VHH, scFv-kih-Fc, or scFv-kh-C_{H}3), or a bispecific fusion protein (e.g. a scFv₂-albumin, scDb-albumin, taFv-toxin, DNL-Fab₃, DNL-Fab₄-IgG, DNL-Fab₄-IgG-cytokine₂). See in particular FIG. 2 of Kontermann MAbs 2012, 4(2): 182-19. See also Brinkmann and Kontermann, MAbs (2017) 9(2): 182-212 (hereby incorporated by reference in its entirety), in particular FIG. 2.

The skilled person is able to design and prepare bispecific antigen-binding molecules. Methods for producing bispecific antigen-binding molecules include chemically crosslinking of antigen-binding molecules or antibody fragments, e.g. with reducible disulphide or non-reducible thioether bonds, for example as described in Segal and Best, 2001. Production of Bispecific Antigen-binding molecules. Current Protocols in Immunology, 14:IV:2.13:2.13.1-2.13.16, which is hereby incorporated by reference in its entirety. For example, N-succinimidyl-3-(-2-pyridyldithio)-propionate (SPDP) can be used to chemically crosslink e.g. Fab fragments via hinge region SH— groups, to create disulfide-linked bispecific F(ab)₂ heterodimers.

Other methods for producing bispecific antigen-binding molecules include fusing antibody-producing hybridomas e.g. with polyethylene glycol, to produce a quadroma cell capable of secreting bispecific antibody, for example as described in D. M, and Bast. B. J. 2001. Production of Bispecific Antigen-binding molecules. Current Protocols in Immunology, 14:IV:2.13:2.13.1-2.13.16.

Bispecific antigen-binding molecules according to the present invention can also be produced recombinantly, by expression from e.g. a nucleic acid construct encoding polypeptides for the antigen binding molecules, for example as described in Antibody Engineering: Methods and Protocols, Second Edition (Humana Press, 2012), at Chapter 40: Production of Bispecific Antigen-binding molecules: Diabodies and Tandem scFv (Hornig and Färber-Schwarz), or French, How to make bispecific antigen-binding molecules, Methods Mol. Med. 2000; 40:333-339, the entire contents of both of which are hereby incorporated by reference.

For example, a DNA construct encoding the light and heavy chain variable domains for the two antigen-binding fragments (i.e. the light and heavy chain variable domains for the antigen-binding fragment capable of binding CD122 or CD132, and the light and heavy chain variable domains for the antigen-binding fragment capable of binding to another target protein), and including sequences encoding a suitable linker or dimerization domain between the antigen-binding fragments can be prepared by molecular cloning techniques. Recombinant bispecific antibody can thereafter be produced by expression (e.g. in vitro) of the construct in a suitable host cell (e.g. a mammalian host cell), and expressed recombinant bispecific antibody can then optionally be purified.

In some embodiments, the antigen-binding molecule comprises an Fv fragment, scFv or Fab fragment specific for CD122 and an Fv, scFv or Fab fragment specific for CD132.

In some embodiments, the antigen-binding molecule according to the present invention comprises:
a CD122-binding region comprising:
   a polypeptide comprising a VH, a CH2 domain and a CH3 domain
   a polypeptide comprising a VL and a CL domain; and
a CD132-binding region comprising:
   a polypeptide comprising a VH, a CH2 domain and a CH3 domain
   a polypeptide comprising a VL and a CL domain.

In some embodiments, the antigen-binding molecule according to the present invention comprises:
a CD122-binding region comprising:
   a polypeptide comprising a VH, a CH1 domain, a CH2 domain and a CH3 domain
   a polypeptide comprising a VL and CL domain; and
a CD132-binding region comprising:
   a polypeptide comprising a VH, a CH1 domain, a CH2 domain and a CH3 domain
   a polypeptide comprising a VL and CL domain.

In some embodiments, the antigen-binding molecule according to the present invention comprises:
a CD122-binding region comprising:
   a polypeptide comprising a VL, a VH, a CH2 domain and a CH3 domain; and
a CD132-binding region comprising:
   a polypeptide comprising a VL, VH, a CH2 domain and a CH3 domain.

In some embodiments, the antigen-binding molecule according to the present invention comprises:
a CD122-binding region comprising:
   a polypeptide comprising a VH, a VL, a CH2 domain and a CH3 domain; and
a CD132-binding region comprising:
   a polypeptide comprising a VH, VL, a CH2 domain and a CH3 domain.

In some embodiments, the antigen-binding molecule according to the present invention comprises:
a CD122-binding region comprising:
   a polypeptide comprising a VL, a VH, a CH1 domain, a CH2 domain and a CH3 domain: and
a CD132-binding region comprising:
   a polypeptide comprising a VL, VH, a CH1 domain, a CH2 domain and a CH3 domain.

In some embodiments, the antigen-binding molecule according to the present invention comprises:
a CD122-binding region comprising:
   a polypeptide comprising a VH, a VL, a CH1 domain, a CH2 domain and a CH3 domain; and
a CD132-binding region comprising:
   a polypeptide comprising a VH, VL, a CH1 domain, a CH2 domain and a CH3 domain.

The variable of the heavy and light chains or the constant regions of the heavy (i.e. CH1) and light chain (CL) regions of a Fab fragment of an antigen-binding molecule according to the invention may be exchanged (i.e. Fab light chain=VL-CH1; Fab heavy chain=VH-CL). Fab fragments formed by association of polypeptides comprising such structure are referred to as “cross-Fab” or “crossover Fab” fragments. In some embodiments, the antigen-binding molecule comprises or consists of, a cross-Fab region which binds to CD122. In some embodiments, the antigen-binding molecule comprises, or consists of, a cross-Fab region which binds to CD132.

In some embodiments, the antigen-binding molecule comprises a cross-Fab fragment specific for CD122 and/or a cross-Fab fragment specific for CD132.

In some embodiments, the antigen-binding molecule according to the present invention comprises:
a CD122-binding region comprising:
   a polypeptide comprising a VH, a CL domain, a CH2 domain and a CH3 domain
   a polypeptide comprising a VL and a CH1 domain; and
a CD132-binding region comprising
   a polypeptide comprising a VH, a CL domain, a CH2 domain and a CH3 domain
   a polypeptide comprising a VL and a CH1 domain.

In some embodiments the antigen-binding molecules of the present invention comprise an Fc region.

In IgG IgA and IgD isotypes Fc regions are composed of CH2 and CH3 regions from one polypeptide, and CH2 and CH3 regions from another polypeptide. The CH2 and CH3 regions from the two polypeptides together form the Fc region. In IgM and IgE isotypes the Fc regions contain three constant domains (CH2, CH3 and CH4), and CH2 to CHA from the two polypeptides together for the Fc region.

Fc regions provide for interaction with Fc receptors and other molecules of the immune system to bring about functional effects. IgG Fc-mediated effector functions are reviewed e.g. in Jefferis et al., Immunol Rev 1998 163:58-76 (hereby incorporated by reference in its entirety), and are brought about through Fc-mediated recruitment and activation of immune cells (e.g. macrophages, dendritic cells, NK cells and T cells) through interaction between the Fc region and Fc receptors expressed by the immune cells, recruitment of complement pathway components through binding of the Fc region to complement protein C1q, and consequent activation of the complement cascade.

In some embodiments, the antigen-binding molecule comprises a polypeptide comprising an amino acid sequence having at least 70%, preferably one of 80%, 85%, 90%, 91%, 92%, 93%, 94%, 95%, 96%, 97%, 98%, 99% or 100% amino acid sequence identity to SEQ ID NO: 444 In some embodiments, the antigen-binding molecule comprises a polypeptide comprising an amino acid sequence having at least 70%, preferably one of 60%, 85%, 90%, 91%, 92%, 93%, 94%, 95%, 96%, 97%, 98%, 99% or 100% amino acid sequence identity to SEQ ID NO. 446. In some embodiments the antigen-binding molecule comprises more than one polypeptide (e.g. 2 polypeptides), each comprising e an amino acid sequence having at least 70%, preferably one of 80%, 85%, 90%, 91%, 92%, 93%, 94%, 95%, 96%, 97%, 98%, 99% or 100% amino acid sequence identity to SEQ ID NO: 444. In some embodiments the antigen-binding molecule comprises more than one polypeptide (e.g. 2 polypeptides), each comprising a an amino acid sequence having at least 70%, preferably one of 80%, 85%, 90%, 91%, 92%, 93%, 94%, 95%, 96%, 97%, 98%, 99% or 100% amino acid sequence identity to SEQ ID NO: 446.

In some embodiments the antigen-binding molecule of the present invention comprises an Fc region comprising modification to reduce antibody effector function Fc-mediated effector functions include Fc receptor binding, antibody-dependent cellular cytotoxicity (ADCC), antibody-dependent cell-mediated phagocytosis (ADCP), complement-dependent cytotoxicity (CDC), formation of the membrane attack complex (MAC), cell degranulation, cytokine and/or chemokine production, and antigen processing and presentation.

Modifications to antibody Fc regions that influence Fc-mediated functions are known in the art, such as those described e.g. in Wang et al., Protein Cell (2018) 9(1). 63-73, which is hereby incorporated by reference in its entirety. In particular, exemplary Fc region modifications known to influence antibody effector function are summarised in Table 1 of Wang et al., Protein Cell (2018) 9(1):63-73.

The combination of substitutions “L234A/L235A” and corresponding substitutions (such as e.g. F234A/L235A in human IgG4) are known to disrupt binding of Fc to Fcγ receptors and inhibit ADCC. ADCP, and also to reduce C1q binding and thus CDC (Schlothauer et al., Protein Engineering, Design and Selection (2016), 29(10):457-466, hereby incorporated by reference in entirety).

In some embodiments the antigen-binding molecule of the present invention comprises an Fc region comprising modification corresponding to the combination of substitutions L234A/L235A.

In some embodiments, the antigen-binding molecule of the present invention comprises an Fc region comprising modification in one or more of the CH2 and CH3 regions promoting association of the FC region. Recombinant co-expression of constituent polypeptides of an antigen-binding molecule and subsequent association leads to several possible combinations. To improve the yield of bispecific antigen-binding molecules of the invention in recombinant production, it is advantageous to introduce in the Fc regions modification(s) promoting association of the desired combination of polypeptides. Suitable modifications are described e.g. in Ha et al., Front. Immnol (2016) 7:384, which is hereby incorporated by reference in its entirety.

In some embodiments the antigen antigen-binding molecule of the present invention comprises an Fc region comprising paired substitutions in the CH3 regions of the Fc region according to one of the following formats, as shown in Table 1 of Ha et al., Front. Immnol (2016) 7:394: KIH, KiHₛ₋ₛ, HA-TF, ZW1, 7.8.60, DD-KK, EW-RVT, EW-RVTₛ₋ₛ SEED or A107.

In some embodiments, the bispecific antigen-binding molecule of the present invention is provided with an Fc region comprising the “knob-into-hole” of “KIH” modification, e.g. as described e.g. in U.S. Pat. No. 7,695,936 and Carter, J Immunol Meth 248, 7-15 (2001). In such embodiments, one of the CHB regions of the Fc region comprises a “knob” modification, and the other CH3 region comprises a “hole” modification. The “knob” and “hole” modifications are positioned within the respective CH3 regions so that the “knob” can be positioned in the “hole” in order to promote heterodimerisation (and inhibit homodimerisation) of the polypeptides and/or stabilise heterodimers. Knobs are constructed by substituting amino acids having small chains with those having larger side chains (e.g. tyrosine or tryptophan). Holes are created by substituting amino acids having large side chains with those having smaller side chains (e.g. alanine of threonine).

In some embodiments, one of the CH3 regions of the Fc region of the antigen-binding molecule of the present invention comprises the substitution (numbering of positions/substitutions in the Fc region herein is according to the EU numbering system as described in Kabat et al., Sequences of Proteins of Immunological interest, 5th Ed. Public Health Service, National Institutes of Health, Bethesda, MD. 1991) T366W, and the other CH3 region of the Fc region comprises the substitution Y407V. In some embodiments, one of the CH3 regions of the Fc region of the antigen-binding molecule comprises the substitution T366W, and the other CH3 region of the Fc region comprises the substitutions T366S and L368A. In some embodiments, one of the CH3 regions of the Fc region of the antigen-binding molecule comprises the substitution T366W, and the other CH3 region of the Fc region comprises the substitutions Y407V, T3666 and L368A.

In some embodiments, one of the CH3 regions comprises the substitution S354G, and the other CH3 region of the Fc region comprises the substitution Y349C. Introduction of these cysteine residues results in formation of a disulfide bridge between the two CH3 regions of the Fe region, further stabilizing the heterodimer (Carter (2001); J Immunol Methods 248, 7-15).

In some embodiments, the Fc region comprises the “KiH_{S-S}” modification. In some embodiments one of the CH3 regions comprises the substitutions T366W and S354C, and the other CH3 region of the Fc region comprises the substitutions T366S, L368A, Y407V and Y349C.

In some embodiments, one of the CHS regions comprises the substitutions K392D and K409D, and the other CH3 region of the Fc region comprises the substitutions E356K and D399K. “DDKK” knob-into-hole technology is described e.g. in WO 2014/131694 A1, and promotes assembly of the heavy chains providing the complementary amino acid residues.

In some embodiments, the antigen-binding molecule of the present invention comprises an Fc region modified as described in Labrijn et al., Proc Natl Acad Sci USA. (2013) 110(13) 5145-50, referred to as Duobody format. In some embodiments one of the CH3 regions comprises the substitution K409R, and the other CH3 region of the Fe region comprises the substitution K405L.

In some embodiments, the antigen-binding molecule of the present invention comprises an Fe region modified as described in Strop et al., J Mol Biol. (2012) 420(3):204-19, so-called ‘EEE-RRR’ format. In some embodiments one of the CH3 regions comprises the substitutions D221E. P22BE and L368E, and the other CH3 region of the Fc region comprises the substitutions D221R. P228R and K409R.

In some embodiments, the antigen-binding molecule comprises an Fc region comprising the “EW-RVT” modification described in Choi et al., Mol Cancer Ther (2013) 12(12):2748-59. In some embodiments one of the CH3 regions comprises the substitutions K360E and K409W, and the other CH3 region of the Fc region comprises the substitutions Q347R, D399V and F4D5T.

In some embodiments, the antigen-binding molecule of the present invention comprises an Fc region comprising the “SEED” modification as described in Davis et al., Protein Eng Des Sel (2010) 23(4): 195-202, in which ß-strand segments of human IgG1 CH3 and IgA CH3 are exchanged.

In some embodiments, one of the CH3 regions comprises the substitutions S364H and F405A, and the other CH3 region of the Fc region comprises the substitutions Y349T and T394F (see e.g. Moore et al. MAbs (2011) 3(6):546-57).

In some embodiments, one of the CH3 regions comprises the substitutions T360V, L351Y, F405A and Y407V, and the other CH3 region of the Fc region comprises the substitutions T360V, T366L, K392L and T394W (see e.g. Von Kreudenstein et al., MAbs (2013) 5(5):646-54).

In some embodiments, one of the CH3 regions comprises the substitutions K360D, 0399M and Y407A, and the other CH3 region of the Fc region comprises the substitutions E345R, Q347R, T366V and K409V (see e.g. Leaver-Fay et al., Structure (2016) 24(4):641-51)

In some embodiments, one of the CH3 regions comprises the substitutions K370E and K409W, and the other CH3 region of the Fc region comprises the substitutions E357N. D399V and F405T (see e.g. Chol et al., PLOS One (2015) 10(12): e0145349).

In particular embodiments, the antigen-binding molecule comprises a KiH Fc region. In particular embodiments, the antigen-binding molecule comprises a KIH_{S-S} Fc region.

In some embodiments the antigen-binding molecule comprises a polypeptide comprising a CH3 region comprising W at the position corresponding to position 366. In some embodiments the antigen-binding molecule comprises a polypeptide comprising a CH3 region comprising W at the position corresponding to position 366 and C al the position corresponding to position 354.

In some embodiments the antigen-binding molecule comprises a polypeptide comprising a CH3 region comprising S at the position corresponding to position 366, and A at the position corresponding to position 368. In some embodiments the antigen-binding molecule comprises a polypeptide comprising a CH3 region comprising S at the position corresponding to position 360. A at the position corresponding to position 368, and Y at the position corresponding to position 407. In some embodiments the antigen-binding molecule comprises a polypeptide comprising a CH3 region comprising S at the position corresponding to position 366. A at the position corresponding to position 368, Y at the position corresponding to position 407, and C at the position corresponding to position 349.

In some embodiments the antigen-binding molecule comprises: (a) a polypeptide comprising a CH3 region comprising W at the position corresponding to position 366, and C at the position corresponding to position 354; and (b) a polypeptide comprising a CH3 region position corresponding to position 366. A at the position corresponding to position 368, Y at the position corresponding to position 407, and C at the position corresponding to position 349.

In some embodiments, the antigen-binding molecule comprises a polypeptide comprising an amino acid sequence having at least 70%, preferably one of 80%, 85%, 90%, 91%, 92%, 93%, 94%, 95%, 96%, 97%, 98%, 99% or 100% amino acid sequence identity to SEQ ID NO: 259. In some embodiments, the antigen-binding molecule comprises a polypeptide comprising an amino acid sequence having at least 70%, preferably one of 80%, 85%, 90%, 91%, 92%, 93%, 94%, 95%, 96%, 97%, 98%, 99% or 100% amino acid sequence identity to SEQ ID NO: 451.

In some embodiments, the antigen-binding molecule comprises a polypeptide comprising an amino acid sequence having at least 70%, preferably one of 80%, 85%, 90%, 91%, 92%, 93%, 94%, 95%, 96%, 97%, 98%, 99% or 100% amino acid sequence identity to SEQ ID NO: 447. In some embodiments, the antigen-binding molecule comprises a polypeptide comprising an amino acid sequence having at least 70%, preferably one of 80%, 85%, 90%, 91%, 92%, 93%, 94% 95%, 96%, 97%, 98%, 99% or 100% amino acid sequence identity to SEQ ID NO: 448. In some embodiments, the antigen-binding molecule comprises a polypeptide comprising an amino acid sequence having at least 70%, preferably one of 80%, 85%, 90%, 91%, 92%, 93%, 94%, 95%, 96%, 97%, 98%, 99% or 100% amino acid sequence identity to SEQ ID NO 447, and a polypeptide comprising an amino acid sequence having at least 70%, preferably one of 80%, 85%, 90%, 91%, 92%, 93%, 94%, 95%, 96%, 97%, 98%, 99% or 100% amino acid sequence identity to SEQ ID NO: 448.

In some embodiments, the antigen-binding molecule comprises a polypeptide comprising an amino acid sequence having at least 70%, preferably one of 80%, 85%, 90%, 91%, 92%, 93%, 94%, 95%, 96%, 97%, 98%, 99% or 100% amino acid sequence identity to SEQ ID NO: 449. In some embodiments, the antigen-binding molecule comprises a polypeptide comprising an amino acid sequence having at least 70%, preferably one of 80%, 85%, 90%, 91%, 92%, 93%, 94%, 95%, 96%, 97%, 98%, 99% or 100% amino acid sequence identity to SEQ ID NO: 450. In some embodiments, the antigen-binding molecule comprises a polypeptide comprising an amino acid sequence having at least 70%, preferably one of 80%, 85%, 90%, 91%, 92%, 93%, 94%, 95%, 96%, 97%, 98%, 99% or 100% amino acid sequence identity to SEQ ID NO: 449, and a polypeptide comprising an amino acid sequence having at least 70%, preferably one of 80%, 85%, 90%, 91%, 92%, 93%, 94%, 95%, 96%, 97%, 98%, 99% or 100% amino acid sequence identity to SEQ ID NO: 450.

In some embodiments, the antigen-binding molecule comprises a polypeptide comprising an amino acid sequence having at least 70%, preferably one of 80%, 85%, 90%, 91%, 92%, 93%, 94%, 95%, 96%, 97%, 98%, 99% or 100% amino acid sequence identity to SEQ ID NO: 452. In some embodiments, the antigen-binding molecule comprises a polypeptide comprising an amino acid sequence having at least 70%, preferably one of 80%, 85%, 90%, 91%, 92%, 93%, 94%, 95%, 96%, 97%, 98%, 99% or 100% amino acid sequence identity to SEQ ID NO: 453. In some embodiments, the antigen-binding molecule comprises a polypeptide comprising an amino acid sequence having at least 70%, preferably one of 80%, 85%, 90%, 91%, 92%, 93%, 94%, 95%, 96%, 97%, 98%, 99% or 100% amino acid sequence identity to SEQ ID NO: 452, and a polypeptide comprising an amino acid sequence having at least 70%, preferably one of 80%, 85%, 90%, 91%, 92%, 93%, 94%, 95%, 96%, 97%, 98%, 99% or 100% amino acid sequence identity to SEQ ID NO: 453.

The present invention also provides polypeptide constituents of the antigen-binding molecules described herein. The polypeptides may be provided in isolated or substantially purified form.

The antigen-binding molecule of the present invention may be, or may comprise, a complex of polypeptides.

In the present specification where a polypeptide comprises more than one domain or region, it will be appreciated that the plural domains/regions can be present in the same polypeptide chain. That is, the polypeptide may comprise more than one domain or region in a fusion polypeptide comprising the domains/regions.

In some embodiments a polypeptide according to the present invention comprises, or consists of, a VH as described herein. In some embodiments a polypeptide according to the present invention comprises, or consists of, a VL as described herein.

In some embodiments, the polypeptide additionally comprises one or more antibody heavy chain constant regions (CH). In some embodiments, the polypeptide additionally comprises one or more antibody light chain constant regions (CL). In some embodiments, the polypeptide comprises a CH1. CH2 region and/or a CH3 region of an immunoglobulin (Ig).

In some embodiments the polypeptide comprises one or more regions of an immunoglobulin heavy chain constant sequence. In some embodiments the polypeptide comprises a CH1 region as described herein.

In some embodiments the polypeptide comprises a CH1-CH2 hinge region as described herein. In some embodiments the polypeptide comprises a CH2 region as described herein. In some embodiments the polypeptide comprises a CH3 region as described herein.

In some embodiments the polypeptide comprises a CH3 region comprising any one of the following amino acid substitutions/combinations of amino acid substitutions (shown e.g. in Table 1 of Ha et al., Front. Immnol (2016) 7:394, incorporated by reference hereinabove); T366W; T366S, L36BA and Y407V; T366W and 5354C; T366S. L368A, Y407V and Y349C; 8364H and F405A; Y349T and T394F; T350V, L351Y, F405A and Y407V; T350V, T366L, K392L and T394W; K360D, D399M and Y407A; E346R, Q347R, T366V and K409V; K409D and K392D; D399K and E356K; K360E and K409W; Q347R, D399V and F405T; K360E, K409W and Y349C; Q347R, D399V, F405T and S354C; K370E and K409W; and E357N, D399V and F405T.

In soma embodiments the CH2 and/or CH3 regions of the polypeptide comprise one or more amino acid substitutions for promoting association of the polypeptide with another polypeptide comprising a CH2 and/or CH3 region.

In some embodiments the polypeptide comprises one or more regions of an immunoglobulin light chain constant sequence. In some embodiments the polypeptide comprises a CL region as described herein.

Also provided by the present invention are antigen-binding molecules comprising a polypeptide according to the present invention.

In some embodiments the antigen-binding molecules and polypeptides of the present invention comprise one or more linker sequences between amino acid sequences. A linker sequence may be provided at one or both ends of one or more of a VH, VL, CH1-CH2 hinge region, CH2 region and a CH3 region of the antigen-binding molecule/polypeptide.

Linker sequences are known to the skilled person, and are described, for example in Chen et al. Adv Drug Deliv Rev (2013) 65(10): 1357-1369, which is hereby incorporated by reference in its entirety. In some embodiments, a linker sequence may be a flexible linker sequence. Flexible linker sequences allow for relative movement of the amino acid sequences which are linked by the linker sequence. Flexible linkers are known to the skilled person, and several are identified in Chen et al., Adv Drug Deliv Rev (2013) 65(10): 1357-1369. Flexible linker sequences often comprise high proportions of glycine and/or serine residues.

In some embodiments, the linker sequence comprises at least one glycine residue and/or at least one serine residue. In some embodiments the linker sequence consists of glycine and serine residues, in some embodiments, the linker sequence has a length of 1-2, 1-3, 1-4, 1-5, 1-10, 1-15, 1-20, 1-30, 1-40 or 1-50 amino acids. In some embodiments a linker sequence comprises, or consists of, one or more copies (e.g. 2, 3 or 4 copies) of the amino acid sequence of SEQ ID NO: 330, 331, 332, 333, 454 or 455.

The antigen-binding molecules and polypeptides of the present invention may additionally comprise further amino acids or sequences of amino acids. For example, the antigen-binding molecules and polypeptides may comprise amino acid sequence(s) to facilitate expression, folding, trafficking, processing, purification or detection of the antigen-binding molecule/polypeptide. For example, the antigen-binding molecule/polypeptide may comprise a sequence encoding a His. (e.g. 6XHis), Myc, GST, MBP, FLAG, HA, E, or Biotin tag, optionally at the N- or G-terminus of the antigen-binding molecule/polypeptide. In some embodiments the antigen-binding molecule/polypeptide comprises a detectable moiety, e.g. a fluorescent, luminescent, immuno-detectable, radio, chemical, nucleic acid of enzymatic label.

The antigen-binding molecules and polypeptides of the present invention may additionally comprise a signal peptide (also known as a leader sequence or signal sequence). Signal peptides normally consist of a sequence of 5-30 hydrophobic amino acids, which form a single alpha helix. Secreted proteins and proteins expressed at the cell surface often comprise signal peptides.

The signal peptide may be present at the N-terminus of the antigen-binding molecule/polypeptide, and may be present in the newly synthesised antigen-binding molecule/polypeptide. The signal peptide provides for efficient trafficking and secretion of the antigen-binding molecule/polypeptide. Signal peptides are often removed by cleavage, and thus are not comprised in the mature antigen-binding molecule/polypeptide secreted from the cell expressing the antigen-binding molecule/polypeptide.

Signal peptides are known for many proteins, and are recorded in databases such as GenBank, UniProt. Swiss-Prot, TrEMBL, Protein Information Resource, Protein Data Bank, Ensembl, and InterPro, and/or can be identified/predicted e.g. using amino acid sequence analysis tools such as SignalP (Petersen et al., 2011 Nature Methods B; 786-786) or Signal-BLAST (Frank and Sippl. 2008 Bioinformatics 24; 2172-2176).

Functional Properties of the Antigen-Binding Molecules

The antigen-binding molecule described herein may be characterised by reference to certain functional properties. In some embodiments, the antigen-binding molecule described herein may possess one or more of the following properties:
binds to CD122;
binds to CD132;
binds to CD122-expressing cells;
binds to CD132-expressing cells;
stimulates signalling by a polypeptide complex comprising CD122 and GD132;
stimulates proliferation of cells expressing CD122 and CD132;
preferentially stimulates proliferation of effector T cells and/or NK cells over regulatory T cells; reduces expression of one or more immune checkpoint proteins (e.g. PD-1);
enhances anticancer activity of cancer antigen-specific immune calls, e.g. in vivo;
unproved thermostability, e.g. as compared to an antigen-binding molecule described in WO 2017/021540 A1.

In some embodiments, the extent of binding of an antigen-binding molecule to an non-target is less than about 10% of the binding of the antibody to the target as measured, e.g., by ELISA, SPR, Bio-Layer Interferometry (BLI), MicroScale Thermophoresis (MST), or by a radioimmunoassay (RIA). Alternatively, the binding specificity may be reflected in terms of binding affinity, where the antigen-binding molecule described herein binds to CD122 and/or CD132 with an affinity that is at least 0.1 order of magnitude greater than the affinity towards a non-target molecule. In some embodiments, the antigen-binding molecule described herein binds to CD122 and/or CD132 with an affinity that is one of at least 0.2, 0.3, 0.4, 0.5, 0.6, 0.7, 0.8, 0.9, 1.0, 1.5, or 2.0 orders of magnitude greater than the affinity towards another, non-target molecule.

Binding affinity of an antigen-binding molecule for its target is often described in terms of its dissociation constant (K_{D}). Binding affinity can be measured by methods known in the art, such as by ELISA, Surface Plasmon Resonance (SPR; see e.g. Hearty et al., Methods Mol Biol (2012) 907:411-442; or Rich et al., Anal Biochem. 2008 Feb. 1; 373(1):112-20), Bio-Layer Interferometry (see e.g. Lad et al., (2015) J Biomol Screen 20(4): 498-507; or Concepcion et al., Comb Chem High Throughput Screen. 2009 September; 12(8): 791-800). MicroScale Thermophoresis (MST) analysis (see e.g. Jerabek-Willemsen et al., Assay Drug Dev Technol. 2011 August; 9(4): 342-353), or by a radiolabelled antigen-binding assay (RIA).

In some embodiments, the antigen-binding molecule described herein binds to CD122 with a K_{D} of 10 μM or less, preferably one of ≤5 μM, ≤2 μM, ≤1 μM, ≤500 nM, ≤100 nM, ≤75 nM, ≤50 nM, ≤40 nM, ≤30 nM, ≤20 nM, ≤15 nM, ≤12.5 nM, ≤10 nM, 59 nM, ≤8 nM, ≤7 nM, ≤6 nM, ≤5 nM, ≤4 nM, ≤3 nM, ≤2 nM, ≤1 nM, ≤500 μM. In some embodiments, the antigen-binding molecule described herein binds to CD132 with a K_{D} of 10 μM or less, preferably one of ≤5 μM, ≤2 μM, ≤1 μM, ≤500 nM, ≤100 nM, ≤75 nM, ≤50 nM, ≤40 nM, ≤30 nM, ≤20 nM, ≤16 nM, ≤ 12.5 nM, ≤10 nM, ≤9 nM, ≤8 nM, ≤7 nM, ≤6 nM, ≤5 nM, ≤4 nM, ≤3 nM, ≤2 nM, ≤1 nM, ≤500 pM.

In some embodiments, the antigen-binding molecule described herein binds to CD122 with an affinity of binding (e.g. as determined by ELISA) of EC50=1000 ng/ml or less, preferably one of ≤900 ng/ml, ≤800 ng/ml, ≤700 ng/ml, ≤600 ng/ml, ≤800 ng/ml, ≤400 ng/ml, ≤300 ng/ml, ≤200 ng/ml, ≤100 ng/ml, ≤90 ng/ml, ≤80 ng/ml, ≤70 ng/ml, ≤60 ng/ml, ≤50 ng/ml, ≤40 ng/ml, ≤30 ng/ml, ≤20 ng/ml, ≤15 ng/ml, ≤10 ng/ml; ≤7.5 ng/ml, ≤5 ng/ml, ≤2.5 ng/ml, or ≤1 ng/ml. In some embodiments, the antigen-binding molecule described herein binds to CD132 with an affinity of binding (e.g. as determined by ELISA) of EC50=1000 ng/ml or less, preferably one of ≤900 ng/ml, ≤800 ng/ml, ≤700 ng/ml, ≤600 ng/ml, ≤500 ng/ml, ≤400 ng/ml, ≤300 ng/ml, ≤200 ng/ml, ≤100 ng/ml, ≤90 ng/ml, ≤80 ng/ml, ≤70 ng/ml, ≤60 ng/ml, ≤50 ng/ml, ≤40 ng/ml, ≤30 ng/ml, ≤20 ng/ml, ≤15 ng/ml, ≤10 ng/ml, ≤7.5 ng/ml, ≤6 ng/ml, ≤2.5 ng/ml, or ≤1 ng/ml.

Affinity of binding to CD122 and/or CD132 may be analysed in vitro by ELISA assay. Suitable assays are well known in the art and can be performed by the skilled person, for example, as described in Antibody Engineering, vol. 1 (2^{nd} Edn), Springer Protocols, Springer (2010); Part V. pp 657-665.

In some embodiments the binding affinity of the antigen-binding molecule of the present invention to CD122 is greater than the binding affinity of an antigen-binding molecule described in WO 2017/021640 A1. In some embodiments, the antigen-binding molecule described herein binds to CD122 with a K_{D} which is less than 1 times, e.g. ≤0.9 times, ≤0.85 times, ≤0.8 times, ≤0.75 limes, ≤0.7 times, ≤0.65 times, ≤0.6 times, ≤0.55 times, ≤0.5 times, ≤0.45 times, ≤0.4 times, ≤0.35 times, ≤0.3 times, ≤0.25 times, ≤0.2 times, ≤0.15 times or ≤0.1 times the K_{D} of binding to CD122 for an antigen-binding molecule described in WO 2017/021540 A1. In some embodiments the binding affinity of the antigen-binding molecule of the present invention to CD132 is greater than the binding affinity of an antigen-binding molecule described in WO 2017/021540 A1. In some embodiments, the antigen-binding molecule described herein binds to CD132 with a K_{D} which is less than 1 times, e.g. ≤0.9 times, ≤0.85 times, ≤0.8 times, ≤0.75 times, ≤0.7 times, ≤0.65 times, ≤0.6 times, ≤0.55 times, ≤0.5 times, ≤0.45 times, ≤0.4 times, ≤0.35 times, ≤0.3 times, ≤0.25 times, ≤0.2 times, ≤0.15 times or ≤0.1 times the K_{D} of binding to CD132 for an antigen-binding molecule described in WO 2017/021540 A1.

The antigen-binding molecule described herein preferably binds to CD122 in a region of CD122 which is accessible to an antigen-binding molecule (i.e., an extracellular antigen-binding molecule) when CD122 is expressed at the cell surface (i.e. in or at the cell membrane). In some embodiments the antigen-binding molecule described herein is capable of binding to CD122 when CD122 is expressed at the call surface. The antigen-binding molecule described herein preferably binds to CD132 in a region of CD132 which is accessible to an antigen-binding molecule (i.e., an extracellular antigen-binding molecule) when CD132 is expressed at the cell surface (i.e. in or at the cell membrane). In some embodiments the antigen-binding molecule described herein is capable of binding to CD132 when CD132 is expressed at the cell surface.

For example, the antigen-binding molecule may bind to CD122 and/or CD132-expressing cells, such as cells expressing CD122 and CD132 at the cell surface, e g immune cells, lymphocytes, T cells (CD4+ T cells, CD8+ T cells) or NK cells.

The ability of an antigen-binding molecule to bind to a given cell type can be analysed by contacting cells with the antigen-binding molecule, and detecting antigen-binding molecule bound to the cells, e.g. after a washing step to remove unbound antigen-binding molecule. The ability of an antigen-binding molecule to bind to CD132-expressing cells and/or CD122-expressing cells can be analysed by methods such as flow cytometry and immunofluorescence microscopy, e.g. as described in the experimental examples of the present application.

The antigen binding molecules of the present invention may stimulate signalling through the intermediate-affinity IL-2 receptor (i.e, the polypeptide complex comprising CD122 and CD132, and not comprising CD25). The antigen-binding molecules are preferably agonists of signalling through the intermediate-affinity IL-2 receptor.

The ability of a given antigen-binding molecule to stimulate signalling through the intermediate-affinity IL-2 receptor can be evaluated e.g. in an in vitro assay, e.g. as described in Example 4 herein Briefly, cells expressing CD122 and CD132 at the cell surface can be contacted with the antigen-binding molecule, and activation of intracellular signalling can be determined by detecting an increase in the level of phosphorylated STAT5 (i.e. pSTAT5). pSTAT5 can be detected e.g. using antibody- or reporter-based methods as described herein.

In some embodiments the antigen-binding molecule of the present invention is capable of increasing the amount of pSTAT5 to more than 1 times, e.g. ≥1.01 times, ≥1.02 times, ≥1.03 times, ≥1.04 times, ≥1.05 times, ≥1.06 times, ≥1.07 times, ≥1.08 times, ≥1.09 times, ≥1.1 times, ≥1.2 times, ≥1.3 times, ≥1.4 times, ≥1.5 times, ≥1.6 times, ≥21.7 times, ≥1.8 times, ≥1.9 times, ≥2 times, ≥3 times, ≥24 times, ≥25 times, ≥26 times, ≥7 times, ≥8 times, ≥9 times, ≥10 times, ≥20 times, ≥30 times, ≥40 times, ≥50 times, ≥60 times, ≥70 times, ≥80 times, ≥90 times, or ≥100 times the level of pSTAT5 detected following culture in the absence of the antigen-binding molecule, or in the presence of an appropriate control antigen-binding molecule (e.g. isotype-matched control antigen-binding molecule), in a comparable assay.

In some embodiments the antigen-binding molecule of the present invention activates signalling through the intermediate-affinity IL-2 receptor to a greater extent than an antigen-binding molecule described in WO 2017/021540 A1. In some embodiments, culture of cells expressing CD122 and CD132 in the presence of an antigen-binding molecule according to the present invention increases the amount of pSTAT5 to more than 1 times, e.g. ≥1.01 times, ≥1.02 times, ≥1.03 times, ≥1.04 times, ≥1.05 times, ≥1.06 times, ≥1.07 times, ≥1.08 times, ≥1.09 times, ≥1.1 times, ≥1.2 times, ≥1.3 times, ≥1.4 times, ≥1.5 times, ≥1.6 times, ≥1.7 times, ≥1.8 times, ≥1.9 times, ≥2 times, ≥3 times, ≥4 times, ≥5 times, ≥6 times, ≥7 times, ≥8 times, ≥9 times, or ≥10 times the level of pSTAT5 detected following culture in the presence of an antigen-binding molecule described in WO 2017/021540 A1, in a comparable assay.

In some embodiments the antigen-binding molecule of the present invention is capable of increasing proliferation of cells expressing CD122 and CD132.

Cell proliferation can be determined by analysing cell division over a period of time. Cell division can be analysed, for example, by in vitro analysis of incorporation of 3H-thymidine or by CFSE dilution assay. e.g. as described in Fulcher and Wong, Immunol Cell Biol (1999) 77(6): 558-564, hereby incorporated by reference in entirety. Proliferating cells may also be identified by analysis of incorporation of 5-ethynyl-2′-deoxyuridine (EdU) by an appropriate assay, as described e.g. in Buck et al., Biotechniques, 2008 June; 44(7):927-9, and Sall and Mitchison, PNAS USA 2008 Feb. 19; 105(7): 2415-2420, both hereby incorporated by reference in their entirety, or by alamarBlue dilution assay as described in Example 3 herein (see e.g. Rampersad et al. Sensors (Basel). (2012)12(9): 12347-12360).

In some embodiments the antigen-binding molecule of the present invention is capable of increasing the number or proportion of proliferating cells to more than 1 times, e.g. ≥1.01 times, ≥1.02 times, ≥1.03 times, ≥1.04 times, ≥1.05 times, ≥1.06 times, ≥1.07 times, ≥1.08 times, ≥1.09 times, ≥1.1 times, ≥1.2 times, ≥1.3 times, ≥1.4 times, ≥1.5 times, ≥1.6 times, ≥1.7 times, ≥1.8 times, ≥1.9 times, ≥2 times, ≥3 times, ≥4 times, ≥5 times, ≥6 times, ≥7 times, ≥8 times, ≥9 times, ≥10 times, ≥20 times, ≥30 times, ≥40 times, ≥50 times, ≥60 times, ≥70 times, ≥80 times, ≥90 times, or ≥100 times the number/proportion of proliferating detected following culture in the absence of the antigen-binding molecule, or in the presence of an appropriate control antigen-binding molecule (e.g. isotype-matched control antigen-binding molecule), in a comparable assay.

In some embodiments the antigen-binding molecule of the present invention increases the number or proportion of proliferating cells to a greater extent than an antigen-binding molecule described in WO 2017/021540 A1. In some embodiments, culture of cells expressing CD122 and CD132 in the presence of an antigen-binding molecule of the present invention increases the number/proportion of proliferating cells to more than 1 times, e.g. ≥1.01 times, ≥1.02 times, ≥1.03 times, ≥1.04 times, ≥1.05 times, ≥1.06 times, ≥1.07 times, ≥1.08 times, ≥1.09 times, ≥1.1 times, ≥1.2 times, ≥1.3 times, ≥1.4 times, ≥1.5 times, ≥1.6 times, ≥1.7 times, ≥1.8 times, ≥1.9 times, ≥2 times, ≥3 times, ≥4 times, ≥5 times, ≥6 times, ≥7 times, ≥8 times, ≥9 times, or ≥10 times the number/proportion of proliferating cells detected following culture in the presence of an antigen-binding molecule described in WO 2017/021540 A1, in a comparable assay.

In some embodiments culture in the presence of an antigen-binding molecule of the present invention causes expansion of cells expressing CD122 and CD132 to a number of cells which is more than 1 times. e.g. ≥1.01 times, ≥1.02 times, ≥1.03 times, ≥1.04 times, ≥1.05 times, ≥1.06 times, ≥1.07 times, ≥1.08 times, ≥1.09 times, ≥1.1 times, ≥1.2 times, ≥1.3 times, ≥1.4 times, ≥1.5 times, ≥1.6 times, ≥1.7 times, ≥1.8 times, ≥1.9 times, ≥2 times, ≥3 times, ≥4 times, ≥5 times, ≥6 times, ≥7 times, ≥8 times, ≥9 times, ≥10 times, ≥20 times, ≥30 times, ≥40 times, ≥50 times, ≥60 times, ≥70 times, 880 times, ≥90 times, or ≥100 times the number of cells detected following culture in the absence of the antigen-binding molecule, or in the presence of an appropriate control antigen-binding molecule (e.g. isotype-matched control antigen-binding molecule).

In some embodiments the antigen-binding molecule of the present invention causes expansion of cells expressing CD122 and CD132 to a greater extent than an antigen-binding molecule described in WO 2017/021540 A1. In some embodiments, culture of cells expressing CD122 and CD132 in the presence of an antigen-binding molecule of the present invention causes expansion of the cells to a number of cells which is more than 1 times, e.g. 21.01 times, ≥1.02 times, ≥1.03 times, ≥1.04 times, ≥1.05 times, ≥1.1 times, ≥1.2 times, ≥1.3 times, ≥1.4 times, ≥1.5 times, ≥1.6 times, ≥1.7 times, ≥1.8 times, ≥1.9 times, ≥2 times, ≥3 times, ≥4 times, ≥5 times, ≥6 times, ≥7 times, ≥8 times, ≥9 times, or ≥10 times the number of cells detected following culture in the presence of an antigen-binding molecule described in WO 2017/021540 A1. In a comparable assay.

In some embodiments the antigen-binding molecule of the present invention preferentially stimulates proliferation/expansion of one or more of the following cell types over (i.e. in preference to) regulatory T cells (e.g. CD4+CD25+FoxP3+ T cells); antigen-specific T cells (e.g. virus-specific T cells), antigen-specific CD4 T cells, antigen-specific CD8 T cells, effector memory CD4 T cells, effector memory CD8 T cells, central memory CD4 T cells, central memory CD8 T cells, cytotoxic CD8+ T cells (i.e. CTLs), NK cells, antigen-specific NK calls, or cells comprising/expressing a chimeric antigen receptor (CAR) or nucleic acid encoding a CAR.

In some embodiments the antigen-binding molecule of the present invention is capable of reducing expression of one or more immune checkpoint proteins. In some embodiments the antigen-binding molecule is capable of reducing expression of one or more immune checkpoint proteins by immune cells. e.g. T cells. Immune checkpoint proteins are well known to the skilled person, and include e.g. PD-1, CTLA-4, LAG-3, TIM-3, VISTA, TIGIT and BTLA.

In some embodiments the antigen-binding molecule of the present invention is capable of reducing expression of PD-1. The ability of an antigen-binding molecule to reduce the expression of an immune checkpoint protein can be analysed by contacting a population of immune cells with the antigen-binding molecule, and subsequently analysing the cells for expression of the immune checkpoint protein, e.g. by flow cytometry. The cells may be contacted with the antigen-binding molecule in vivo, e.g. through administration of the antigen-binding molecule to a subject, or cells obtained from a subject may be contacted in vitro or ex vivo with the antigen-binding molecule.

In some embodiments the antigen-binding molecule of the present invention causes a reduction in the level expression of PD-1 by T cells to less than less than 1 times, e.g. ≤0.99 hires, ≤0.95 times, ≤0.9 times, ≤0.85 times, ≤0.8 times, ≤0.75 times, ≤0.7 times, ≤0.65 times, ≤0.6 times, ≤0.55 times, ≤0.5 times, ≤0.45 times, ≤0.4 times, ≤0.35 times, ≤0.3 times, ≤0.26 times, ≤0.2 times, ≤0.15 times, ≤0.1 times, ≤0.05 times, or ≤0.01 times the level of expression by PD-1 by T cells observed in the absence of the antigen-binding molecule (or in the presence of an appropriate control antigen-binding molecule), in a given assay.

In some embodiments the antigen-binding molecule of the present invention enhances anticancer activity of cancer antigen-specific immune cells, e.g. in vivo. The ability of an antigen-binding molecule to enhance anticancer immune response can be analysed e.g. as described in Example 9 herein.

In some embodiments, the antigen-binding molecule of the present invention may display improved thermostability, e.g. as compared to an antigen-binding molecule described in WO 2017/021540 A1.

Thermostability of antigen-binding molecules can be analysed by methods well known to the skilled person, including Differential Scanning Fluorimetry and Differential Scanning calorimetry (DSC), which are described e.g. in He et al., J Pharm Sci. (2010) which is hereby incorporated by reference in its entirety.

In some embodiments, the antigen-binding molecule of the present invention may be determined in such an assay to have a Tₘ1 value which is more than 1 times, e.g. ≥1.01 times, ≥1.02 times, ≥1.03 times, ≥1.04 times, ≥1.06 times, ≥1.06 times, ≥1.07 times, ≥1.08 times, ≥1.09 times, ≥1.1 times, ≥1.2 times, ≥1.3 times, ≥1.4 times, ≥1.5 times, ≥1.6 times, ≥1.7 times, ≥1.6 times, ≥1.9 times, ≥2 times, ≥3 times, ≥4 times, ≥5 times, ≥6 times, ≥7 times, ≥8 times, ≥9 times, or ≥10 times the Tₘ1 value determined for an antigen-binding molecule described in WO 2017/021540 A1.

Membrane Anchored Antigen-Binding Molecules

In some embodiments, the antigen-binding molecule of the present invention further comprises a cell membrane anchor region. As used herein, a ‘cell membrane anchor region’ is a region providing for anchoring of the antigen-binding molecule to the cell membrane of a cell expressing the antigen-binding molecule. ‘Anchoring’ may be reversible or irreversible.

In some embodiments the cell membrane anchor region is a transmembrane domain. A transmembrane domain refers to any three-dimensional structure formed by a sequence of amino acids which is thermodynamically stable in a biological membrane, e.g. a cell membrane.

The transmembrane domain may comprise or consist of a sequence of amino acids which forms a hydrophobic alpha helix or beta-barrel. The amino acid sequence of the transmembrane domain may be, or may be derived from, the amino acid sequence of a transmembrane domain of a protein comprising a transmembrane domain. Transmembrane domains are recorded in databases such as GenBank, UniProt. Swiss-Prot. TrEMBL, Protein Information Resource, Protein Data Bank, Ensembl, and InterPro, and/or can be identified/predicted e.g. using amino acid sequence analysis tools such as TMHMM (Krogh et al. 2001 J Mol Biol 305; 567-580).

In some embodiments, the amino acid sequence of the transmembrane domain may be, or may be derived from, the amino acid sequence of the transmembrane domain of a protein expressed at the cell surface. In some embodiments the protein expressed at the cell surface is a receptor or ligand, e.g. an immune receptor or ligand. In some embodiments the amino acid sequence of the transmembrane domain may be, or may be derived from, the amino acid sequence of the transmembrane domain of one of ICOS, ICOSL, CD86, CTLA-4, CD28, CD80, MHC class I α, MHC class II α, MHC class II β, CD3ε, CD3δ, CD3γ, CD3-ζ, TCRα, TCRβ, CD4, CD8α, CD8β, CD40, CD40L, PD-1, PD-L1, PD-L2, 4-1BB, 4-1BBL, OX40, OX40L, GITR, GITRL, TIM-3, Galectin 9, LAG3, CD27, CD70, LIGHT, HVEM, TIM-4, TIM-1, ICAM1 LFA-1, LFA-3, CD2, BTLA, CD160, LILRB4, LILRB2, VTCN1, CD2, CD4B, 284 SLAM, CD30, CD30L, DR3, TL1A, CD226, CD155, CD112 and CD276.

In some embodiments, the cell membrane anchor region may be a lipid anchor region. In some embodiments, a lipid anchor region comprises or consists of a lipid anchor (e.g. a GPI anchor). A lipid anchor refers to a moiety capable of associating (e.g. covalently) with the lipid component of a biological membrane (e.g. cell membrane). Through such association, a protein having a lipid anchor attached thereto is ‘anchored’ in the cell membrane. A lipid anchor typically comprises a lipophilic group. Lipid anchors, lipophilic groups thereof and modification of proteins to attach lipid anchors is described for example in Resh 2013, Curr Biol. 23(10); R431-R435, which is hereby incorporated by reference in its entirety. A lipid anchor may comprise or consist of an isoprenyl, myristoyl, palmitoyl, fatty acyl, diacylglycerol, steroyl, or phospholipid group, or a glycosylphosphatidyl inositol (GPI) anchor.

In some embodiments, a lipid anchor region comprises or consists of a lipid anchor signal sequence. A lipid anchor signal sequence refers to an amino acid sequence directing processing of a protein to attach a lipid anchor. Following such processing the antigen-binding molecule comprises a lipid anchor.

Chimeric Antigen Receptors (CARs)

The present invention also provides a chimeric antigen receptor (CAR) comprising an antigen-binding molecule according to the present invention.

Chimeric Antigen Receptors (CARs) are recombinant receptors that provide both antigen-binding and T cell activating functions. CAR structure and engineering is reviewed, for example, in Dotti et al., Immunol Rev (2014) 257(1), hereby incorporated by reference in its entirety.

CARs comprise an antigen-binding region linked to a cell membrane anchor region and a signaling region. An optional hinge region may provide separation between the antigen-binding region and cell membrane anchor region, and may act as a flexible linker. The antigen-binding region of a CAR may be based on the antigen-binding region of an antibody which is specific for the antigen to which the CAR is targeted, or other agent capable of binding to the target. For example, the antigen-binding domain of a CAR may comprise amino acid sequences for the complementarity-determining regions (CDRs) or complete light chain and heavy chain variable region amino acid sequences of an antibody which binds specifically to the target protein. Antigen-binding domains of CARs may target antigen based on other protein:protein interaction, such as ligand:receptor binding; for example an IL-13Rα2-targeted CAR has been developed using an antigen-binding domain based on IL-13 (see e.g. Kahlon et al. 2004 Cancer Res 64(24): 9160-9166).

The antigen-binding region of the CAR of the present invention may be provided with any suitable format. e.g. scfv, Fab, etc.

The cell membrane anchor region is provided between the antigen-binding region and the signalling region of the CAR. The cell membrane anchor region provides for anchoring the CAR to the cell membrane of a cell expressing a CAR, with the antigen-binding region in the extracellular space, and signalling region inside the cell. In some embodiments, the CAR of the present invention comprises a cell membrane anchor region comprising or consisting of an amino acid sequence which comprises, consists of, or is derived from, the transmembrane region amino acid sequence for one of CD3-ζ, CD4, CD8 or CD28. As used herein, a region which is ‘derived from’ a reference amino acid sequence comprises an amino acid sequence having at least 60%, e.g. one of at least 65%, 70%, 75%, 80%, 85%, 90%, 91%, 92%, 93%, 94%, 95%, 96%, 97%, 98%, 99% or 100% sequence identity to the reference sequence.

The signalling region of a CAR allows for activation of the T cell. The CAR signalling regions may comprise the amino acid sequence of the intracellular domain of CD3-ζ, which provides immunoreceptor tyrosine-based activation motifs (ITAMs) for phosphorylation and activation of the CAR-expressing T cell. Signaling regions comprising sequences of other ITAM-containing proteins have also been employed in CARs, such as domains comprising the ITAM containing region of FcγRI (Haynes et al., 2001 J Immunol 166(1): 182-187). CARs comprising a signalling region derived from the intracellular domain of CD3-ζ are often referred to as first generation CARs. Signalling regions of CARs may also comprise co-stimulatory sequences derived from the signalling region of co-stimulatory molecules, to facilitate activation of CAR-expressing T cells upon binding to the target protein. Suitable co-stimulatory molecules include CD28, OX40, 4-18B, ICOS and CD27. CARs having a signalling region including additional co-stimulatory sequences are often referred to as second generation CARs.

In some cases CARs are engineered to provide for co-stimulation of different intracellular signalling pathways. For example, signalling associated with CD28 costimulation preferentially activates the phosphatidylinositol 3-kinase (P13K) pathway, whereas the 4-1BB-mediated signalling is through TNF receptor associated factor (TRAF) adaptor proteins. Signalling regions of CARs therefore sometimes contain co-stimulatory sequences derived from signalling regions of more than one co-stimulatory molecule. CARs comprising a signalling region with multiple co-stimulatory sequences are often referred to as third generation CARs. In some embodiments, the CAR of the present invention comprises one or more co-stimulatory sequences comprising or consisting of an amino acid sequence which comprises, consists of, or is derived from, the amino acid sequence of the intracellular domain of one or more of CD28, OX40, 4-1BB. ICOS and CD27.

An optional hinge region may provide separation between the antigen-binding domain and the transmembrane domain, and may act as a flexible linker. Hinge regions may be flexible domains allowing the binding moiety to orient in different directions. Hinge regions may be derived from IgG1 or the CH2CH3 region of immunoglobulin. In some embodiments, the CAR of the present invention comprises a hinge region comprising or consisting of an amino acid sequence which comprises, consists of, or is derived from, the amino acid sequence of the hinge region of IgG1 or the CH2CH3 region of immunoglobulin. CARs may be combined with costimulatory ligands, chimeric costimulatory receptors or cytokines to further enhance T cell potency, specificity and safety (Sadelain et al., The basic principles of chimeric antigen receptor (CAR) design. Cancer Discov. 2013 April; 3(4): 388-398 doi: 10.1168/2159-8290 CD-12-0548, specifically incorporated herein by reference).

Also provided is a cell comprising a CAR according to the invention. The CAR according to the present invention may be used to generate CAR-expressing immune cells, e.g. CAR-T or CAR-NK cells. Engineering of CARs into immune cells may be performed during culture, in vitro, for transduction and expansion, such as happens during expansion of T cells for adoptive T cell therapy.

Nucleic Acids and Expression Vectors

The present invention provides a nucleic acid encoding an antigen-binding molecule or CAR according to the present invention. In some embodiments, the nucleic acid is purified or isolated, e.g. from other nucleic acid, or naturally-occurring biological material.

The present invention also provides a vector comprising nucleic acid encoding an antigen-binding molecule or CAR according to the present invention.

The nucleic acid and/or vector according to the present invention may be provided for introduction into a cell, e.g. a primary human immune cell. Suitable vectors include plasmids, binary vectors, DNA vectors, mRNA vectors, viral vectors (e.g. gammaretroviral vectors (e.g. murine Leukemia virus (MLV)-derived vectors), lentiviral vectors, adenovirus vectors, adeno-associated virus vectors, vaccinia virus vectors and herpesvirus vectors), transposon-based vectors, and artificial chromosomes (e.g. yeast artificial chromosomes), e.g. as described in Maus et al., Annu Rev Immunol (2014) 32:189-225 or Morgan and Boyerinas, Biomedicines 2016 4, 9, which are both hereby incorporated by reference in their entirety. In some embodiments, the viral vector may be a lentiviral, retroviral, adenoviral, or Herpes Simplex Virus vector. In some embodiments, the lentiviral vector may be pELNS, or may be derived from pELNS. In some embodiments, the vector may be a vector encoding CRISPR/Cas9.

In some embodiments, the nucleic acid according to the present invention comprises, or consists of, a nucleic acid sequence having at least 60%, 65%, 70%, 75%, 80%, 85%, 86%, 87%, 88%, 89%, 90%, 91%, 92%, 93%, 94%, 95%, 96%, 97%, 98%, 99%, or 100% sequence identity to one of SEQ ID NOs:335 to 433, or a nucleic acid sequence encoding the same amino acid sequence as one of SEQ ID NOs:335 to 433 as a result of codon degeneracy.

Cells Comprising/Expressing the Antigen-Binding Molecules/CARs

The present invention also provides a cell comprising or expressing an antigen-binding molecule or CAR according to the present invention. Also provided is a cell comprising or expressing a nucleic acid or expression vector according to the invention.

The cell may be a eukaryotic cell, e.g. a mammalian cell. The mammal may be a human, or a non-human mammal (e.g. rabbit, guinea pig, rat, mouse or other rodent (including any animal in the order Rodentia), cat, dog, pig, sheep, goat, cattle (including cows, e.g. dairy cows, or any animal in the order Bos), horse (including any animal in the order Equidae), donkey, and non-human primate).

In some embodiments, the cell may be from, or may have been obtained from, a human subject.

The cell may be an immune cell. The cell may be a cell of hematopoietic origin, e.g. a neutrophil, eosinophil, basophil, dendritic cell, lymphocyte, or monocyte. The lymphocyte may be e.g. a T cell, B cell, NK cell, NKT cell or innate lymphoid cell (ILC), or a precursor thereof. The cell may express e.g. CD3 polypeptides (e.g. CD3γ CD3ε CD3ζ or CD3δ). TCR polypeptides (TCRα or TCRβ), CD27, CD26, CD4 or CD8. In some embodiments, the cell is a T cell. In some embodiments, the T cell is a CD3+ T cell, in some embodiments, the T cell is a CD3+, CD8+ T cell. In some embodiments, the T cell is a cytotoxic T cell (e.g. a cytotoxic T lymphocyte (CTL)).

In some embodiments, the cell is an antigen-specific T cell, in embodiments herein, an “antigen-specific” T cell is a cell which displays certain functional properties of a T cell in response to the antigen for which the T cell is specific, or a cell expressing said antigen. In some embodiments, the properties are functional properties associated with effector T cells, e.g. cytotoxic T cells, In some embodiments, an antigen-specific T cell may display one or more of the following properties, cytotoxicity, e.g. to a cell comprising/expressing antigen for which the T cell is specific; proliferation, IFNγ expression, CD107a expression, IL-2 expression, TNFα expression, perforin expression, granzyme expression, granulysin expression, and/or FAS ligand (FASL) expression, e.g. in response to antigen for which the T cell is specific or a cell comprising/expressing antigen for which the T cell is specific. In some embodiments, the antigen for which the T cell is specific may be a peptide or polypeptide of a virus, e.g. Epstein-Barr virus (EBV), influenza virus, measles virus, hepatitis B virus (HBV), hepatitis C virus (HCV), human immunodeficiency virus (HIV), lymphocytic choriomeningitis virus (LCMV), Herpes simplex virus (HSV) or human papilloma virus (HPV).

The present invention also provides a method for producing a cell comprising a nucleic acid or expression vector according to the present invention, comprising introducing a nucleic acid or expression vector according to the present invention into a cell. The present invention also provides a method for producing a cell expressing an antigen-binding molecule or CAR, according to the present invention, comprising introducing a nucleic acid or expression vector according to the present invention in a cell. In some embodiments, the methods additionally comprise culturing the cell under conditions suitable for expression of the nucleic acid or expression vector by the cell. In some embodiments, the methods are performed in vitro.

In some embodiments, introducing an isolated nucleic acid or expression vector according to the invention into a cell comprises transduction, e.g. retroviral transduction. Accordingly, in some embodiments the isolated nucleic acid or expression vector is comprised in a viral vector, or the vector is a viral vector. In some embodiments, the method comprises introducing a nucleic acid or expression vector according to the invention by electroporation, e.g. as described in Koh et al., Molecular Therapy—Nucleic Acids (2013) 2, e114, which is hereby incorporated by reference in its entirety.

The present invention also provides cells obtained or obtainable by the methods according to the present invention.

Producing the Antigen-Binding Molecules and CARS

Antigen-binding molecules and CARs according to the invention may be prepared according to methods for the production of polypeptides known to the skilled person.

The polypeptide(s) of interest may be prepared by chemical synthesis, e.g. liquid or solid phase synthesis. For example, peptides/polypeptides can by synthesised using the methods described in, for example, Chandrudu et al., Molecules (2013), 18; 4373-4388, which is hereby incorporated by reference in its entirety. Alternatively, antigen-binding molecules and CARs according the invention may be produced by recombinant expression Molecular biology techniques suitable for recombinant production are well known in the art, such as those set out in Green and Sambrook, Molecular Cloning: A Laboratory Manual (4th Edition). Cold Spring Harbor Press, 2012, which is hereby incorporated by reference in its entirety.

Expression may be from a nucleotide sequence. The nucleotide sequence may be contained in a vector. A “vector” as used herein is an oligonucleotide molecule (DNA or RNA) used as a vehicle to transfer foreign genetic material into a cell. The vector may be an expression vector for expression of the foreign genetic material in the cell. Such vectors may include a promoter sequence operably linked to the nucleotide sequence encoding the sequence to be expressed. A vector may also include a termination codon and expression enhancers. Any suitable vectors, promoters, enhancers and termination codons known in the art may be used to express a peptide or polypeptide from a vector according to the invention. In some embodiments, the vector may be a plasmid, MAC, virus, etc. In some embodiments, the vector may be a eukaryotic expression vector, e.g. a vector comprising the elements necessary for expression of protein from the vector in a eukaryotic cell. In some embodiments, the vector may be a mammalian expression vector, e.g. comprising a cytomegalovirus (CMV) or SV40 promoter to drive protein expression.

The term “operably linked” may include the situation where a selected nucleotide sequence and regulatory nucleotide sequence (e.g. promoter and/or enhancer) are covalently linked in such a way as to place the expression of the nucleotide sequence under the influence or control of the regulatory sequence (thereby forming an expression cassette). Thus a regulatory sequence is operably linked to the selected nucleotide sequence if the regulatory sequence is capable of effecting transcription of the nucleotide sequence. The resulting transcript may then be translated into a desired peptide or polypeptide.

In some cases the antigen-binding molecules according to the present invention are comprised of more than one polypeptide chain. In such cases, production of the antigen-binding molecules may comprise transcription and translation of more than one polypeptide chain, and subsequent association of the polypeptide chains to form the antigen-binding molecule.

For recombinant production according to the invention, any cell suitable for the expression of polypeptides may be used. The cell may be a prokaryote or eukaryote. In some embodiments the cell is a prokaryotic cell, such as a cell of archaes or bacteria. In some embodiments the bacteria may be Gram-negative bacteria such as bacteria of the family Enterobacteriaceae, for example Escherichia coli.

In some embodiments, the cell is a eukaryotic cell such as a yeast cell, a plant cell, insect cell or a mammalian call, e.g. CHO, HEK, HeLa or COS cells.

In some cases the cell is not a prokaryotic cell because some prokaryotic cells do not allow for the same fokling or post-translational modifications as eukaryotic cells. In addition, very high expression levels are possible in eukaryotes and proteins can be easier to purify from eukaryotes using appropriate tags. Specific plasmids may also be utilised which enhance secretion of the protein into the media.

Production may involve culture or fermentation of a eukaryotic cell modified to express the peptide or polypeptide. The culture or fermentation may be performed in a bioreactor provided with an appropriate supply of nutrients, air/oxygen and/or growth factors. Secreted proteins can be collected by partitioning culture media/fermentation broth from the cells, extracting the protein content, and separating individual proteins to isolate secreted peptide or polypeptide. Culture, fermentation and separation techniques are well known to those of skill in the art, and are described, for example, in Green and Sambrook, Molecular Cloning: A Laboratory Manual (4th Edition; incorporated by reference herein above).

Bioreactors include one or more vessels in which cells may be cultured. Culture in the bioreactor may occur continuously, with a continuous flow of reactants into, and a continuous flow of cultured calls from, the reactor. Alternatively, the culture may occur in batches. The bioreactor monitors and controls environmental conditions such as pH, oxygen, flow rates into and out of, and agitation within the vessel such that optimum conditions are provided for the cells being cultured.

Following culture of cells that express the antigen-binding molecule or CAR, the polypeptide of interest is preferably isolated. Any suitable method for separating proteins from cell culture known in the art may be used, in order to isolate the polypeptide from a culture, it may be necessary to first separate the cultured cells from media containing the polypeptide of interest. If the polypeptide of interest is secreted from the cells, the cells may be separated from the culture media that contains the secreted polypeptide of interest by centrifugation. If the polypeptide of interest collects within the cell it will be necessary to disrupt the cells prior to centrifugation, for example using sonification, rapid freeze-thaw or osmotic lysis Centrifugation will produce a pellet containing the cultured cells, or cell debris of the cultured cells, and a supernatant containing culture medium and the polypeptide of interest.

It may then be desirable to isolate the polypeptide of interest from the supernatant or culture medium, which may contain other protein and non-protein components. A common approach to separating protein components from a supernatant or culture medium is by precipitation. Proteins of different solubilities are precipitated at different concentrations of precipitating agent such as ammonium sulfate. For example, at low concentrations of precipitating agent, water soluble proteins are extracted. Thus, by adding different increasing concentrations of precipitating agent, proteins of different solubilities may be distinguished. Dialysis may be subsequently used to remove ammonium sulfate from the separated proteins.

Other methods for distinguishing different proteins are known in the art, for example ion exchange chromatography and size chromatography. These may be used as an alternative to precipitation, or may be performed subsequently to precipitation.

Once the polypeptide of interest has been isolated from culture it may be desired or necessary to concentrate the peptide or polypeptide. A number of methods for concentrating proteins are known in the art, such as ultrafiltration or lyophilisation.

Generating/Expanding Populations of Immune Cells

Antigen-binding molecules according to the present invention also find use in methods for generating/expanding populations of immune cells. Essentially, the antigen-binding molecules according to the present invention find use in generating/expanding populations of cell types expressing CD122 and CD132 (e.g. at the cell surface).

The cells may be e.g. T cells, antigen-specific T cells (e.g. virus-specific T cells), antigen-specific CD4 T cells, antigen-specific CD8 T cells, effector memory CD4 T cells, effector memory CD8 T cells, central memory CD4 T cells, central memory CD8 T cells, cytotoxic CD8+ T cells (i.e. CTLs). NK cells or antigen-specific NK cells.

The cells may be antigen-specific immune cells, e.g. antigen-specific T cells. For example, the cells may be specific for a peptide/polypeptide of a virus, e.g. adenovirus, Epstein-Barr virus (EBV), cytomegalovirus (CMV), human papilloma virus (HPV), influenza virus, measles virus, hepatitis B virus (HBV), hepatitis C virus (HCV), human immunodeficiency virus (HIV), lymphocytic choriomeningitis virus (LCMV), or herpes simplex virus (HSV) Virus-specific immune cells may be an adenovirus-specific T cells (AdVSTs). Epstein-Barr virus-specific T cells (EBVSTs), cytomegalovirus-specific T cells (CMVSTs), human papilloma virus-specific T cells (HPVSTs), influenza virus-specific T cells, measles virus-specific T cells, hepatitis B virus-specific T cells (HBVSTs), hepatitis C virus-specific T cells (HCVSTs), human immunodeficiency virus-specific T cells (HIVSTs), lymphocytic choriomeningitis virus-specific T cells (LCMVSTs), or herpes simplex virus-specific T cells (HSVSTs).

The cells may comprise/express a chimeric antigen receptor (CAR) or nucleic acid encoding a CAR. The cells may comprise/express a TGFβ decoy receptor, or nucleic acid encoding a TGFβ decoy receptor.

The methods comprise contacting cells expressing CD122 and CD132 in the presence of an antigen-binding molecule according to the present invention. The cells expressing CD122 and CD132 are stimulated by the antigen-binding molecule to undergo cell division (i.e. proliferate), resulting in an increase in the number of cells.

In some embodiments, the methods comprise generating/expanding cells in vitro. In some embodiments, the methods comprise generating/expanding cells ex vivo. In some embodiments the methods comprise culturing cells in vitro in the presence of an antigen-binding molecule according to the present invention.

Culture of cells may be performed using suitable medium and under suitable environmental conditions (e.g. temperature, pH, humidity, atmospheric conditions, agitation etc.) for the in vitro culture of immune cells, which are well known to the person skilled in the art of cell culture. Conveniently, cultures of cells may be maintained at 37° C. In a humidified atmosphere containing 5% CO₂. Cultures can be performed in any vessel suitable for the volume of the culture, e.g. in wells of a cell culture plate, cell culture flasks, a bioreactor, etc. The cell cultures can be established and/or maintained at any suitable density, as can readily be determined by the skilled person. In some embodiments cells are cultured in a bioreactor. In some embodiments, cells are cultured in a bioreactor described in Somerville and Dudley, Oncoimmunology (2012) 1(8): 1435-1437, which is hereby incorporated by reference in its entirety. In some embodiments cells are cultured in a GRex cell culture vessel, e.g. a GRex flask or a GRex 100 bioreactor.

In some embodiments, immune cells expressing CD122 and CD132 may be generated or expanded from within a population of immune cells. It will be appreciated that the population of immune cells comprises the immune cells expressing CD122 and CD132 The population of immune cells from which the population of immune cells expressing CD122 and CD132 are generated/expanded according to the methods of the present invention comprise at least one immune cell expressing CD122 and CD132.

In some embodiments, immune cells expressing CD122 and CD132 may be generated or expanded from within a population of PBMCs. The methods may involve expansion of T cells (e.g. antigen-specific T cells) from within a population of immune cells (e.g. PBMCs, PBLs). The immune cells (e.g. PBMCs, PBLs) used in the methods of the invention may be freshly obtained, or may be thawed from a sample of immune cells which has previously been obtained and frozen.

In embodiments of the methods disclosed herein, generation or expansion of a population of immune cells may involve culture of a population of PBMCs. In some embodiments, a population of immune cells may be generated/expanded from within a population of T cells (e.g. a population of T cells of heterogeneous type and/or specificity), which may have been obtained from a blood sample or a population of PBMCs. Culture of the population of immune cells from which the calls expressing CD122 and CD132 are generated/expanded may result in an increase of the number of cells expressing CD122 and CD132, and/or result in an increased proportion of such cells in the cell population at the end of the culture.

In some embodiments, the methods comprise treating cells to increase expression (e.g. surface expression) of CD122 and/or CD132, and expansion of cells expressing CD122 and CD132. For example. T cell activation (e.g. by stimulation using anti-CD3 (e.g. clone OKT3) and anti-CD28) induces upregulation of CD122 and CD132. Antigen-specific T cells also have upregulated expression of CD122 and CD132. In some embodiments the methods comprise contacting immune calls with antigen or cells presenting antigen.

In some embodiments, the population of cells is generated/expanded in vivo following administration of an antigen-binding molecule according to the present invention for administration of cells expressing the antigen-binding molecule) to a subject.

Compositions and Formulations

The invention described herein also provides compositions comprising the antigen-binding molecules, nucleic acids, expression vectors and cells described herein.

The antigen-binding molecules, nucleic acids, expression vectors and cells described herein may be formulated as pharmaceutical compositions or medicaments for clinical use and may comprise a pharmaceutically acceptable carrier, diluent, excipient or adjuvant. The composition may be formulated for topical, parenteral, systemic, intracavitary, intravenous, intra-arterial, intramuscular, intrathecal, intraocular, intraconjunctival, intratumoral, subcutaneous, intradermal, intrathecal, oral or transdermal routes of administration which may include injection or infusion. Suitable formulations may comprise the antigen-binding molecule in a sterile or isotonic medium. Medicaments and pharmaceutical compositions may be formulated in flux, including gel, form. Fluid formulations may be formulated for administration by injection or infusion (e.g. via catheter) to a selected region of the human or animal body.

In some embodiments the antigen-binding molecule, nucleic acid, expression vector, CAR, composition or cells according to the present invention are formulated for injection or infusion, e.g. into a blood vessel or tumor.

In accordance with the invention described herein methods are also provided for the production of pharmaceutically useful compositions, such methods of production may comprise one or more steps selected from: isolating an antigen-binding molecule, nucleic acid, expression vector or cells described herein; and/or mixing an antigen-binding molecule, nucleic acid, expression vector or cells described herein with a pharmaceutically acceptable carrier, adjuvant, excipient or diluent.

For example, a further aspect the invention described herein relates to a method of formulating or producing a medicament or pharmaceutical composition for use in the treatment of a cancer, the method comprising formulating a pharmaceutical composition or medicament by mixing an antigen-binding molecule, nucleic acid, expression vector or calls described herein with a pharmaceutically acceptable carrier, adjuvant, excipient or diluent.

Therapeutic and Prophylactic Applications

The antigen-binding molecules, CARs, nucleic acids, expression vectors, cells and compositions described herein find use in therapeutic and prophylactic methods.

The invention described herein provides antigen-binding molecules, nucleic acids, expression vectors, cells and compositions described herein for use in a method of medical treatment or prophylaxis. The invention described herein also provides the use of antigen-binding molecules, nucleic acids, expression vectors, cells and compositions described herein in the manufacture of medicaments for treating or preventing a disease or condition. The invention described herein also provides methods of treating of preventing a disease or condition, comprising administering to a subject a therapeutically or prophylactically effective amount of an antigen-binding molecule, nucleic acid, expression vector, cell or composition described herein.

‘Treatment’ may, for example, be reduction in the development or progression of a disease/condition, alleviation of the symptoms of a disease/condition or reduction in the pathology of a disease/condition. Treatment or alleviation of a disease/condition may be effective to prevent progression of the disease/condition, e.g. to prevent worsening of the condition or to slow the rate of development. In some embodiments treatment or alleviation may lead to an improvement in the disease/condition, e.g. a reduction in the symptoms of the disease/condition or reduction in some other correlate of the severity/activity of the disease/condition. Prevention of a disease/condition may refer to prevention of a worsening of the condition or prevention of the development of the disease/condition, e.g. preventing an early stage disease/condition developing to a later, chronic, stage.

The articles of the present invention find use in the treatment of any disease/condition which would benefit from an increase in the number/frequency of immune cells such as T cells (in particular effector T cells) and/or NK cells. In particular, the antigen-binding molecules and pharmaceutical compositions described herein find use to treat or prevent T cell dysfunctional disorders, cancers and infectious disease.

It will be appreciated that the therapeutic and prophylactic utility of the present invention extends to the treatment of any subject that would benefit in an increase in the number of (i.e. expansion of a population of) cells expressing CD122 and CD132 (e.g. effector T cells and/or NK cells).

T Cell Dysfunctional Disorders

A T cell dysfunctional disorder may be a disease or condition in which normal T cell function is impaired causing downregulation of the subject's immune response to pathogenic antigens, e.g. generated by infection by exogenous agents such as microorganisms, bacteria and viruses, or generated by the host in some disease states such as in some forms of cancer (e.g. in the form of tumor-associated antigens).

The T cell dysfunctional disorder may comprise T cell exhaustion or T cell anergy. T cell exhaustion comprises a state in which CD8+ T cells fail to proliferate or exert T cell effector functions such as cytotoxicity and cytokine (e.g. IFNγ) secretion in response to antigen stimulation Exhausted T cells may also be characterised by sustained expression of one or more markers of T cell exhaustion, e.g. PD-1, CTLA-4, LAG-3, TIM-3. Thus, in some cases the antigen-binding molecules and pharmaceutical compositions described herein find use to treat or prevent T cell dysfunctional disorders, cancers and infectious disease, wherein treatment with the antigen-binding molecules and pharmaceutical compositions results in reduced expression by T cells of one or more markers of T cell exhaustion. In some cases, the treatment results in reduced expression by T cells of PD-1.

The T cell dysfunctional disorder may be manifest as an infection, or inability to mount an effective immune response against an infection. The infection may be chronic, persistent, latent or slow, and may be the result of bacterial, viral, fungal or parasitic infection. As such, treatment may be provided to patients having a bacterial, viral or fungal infection. Examples of bacterial infections include infection with Helicobacter pylori. Examples of viral infections include infection with HIV, hepatitis B or hepatitis C.

The T-cell dysfunctional disorder may be associated with a cancer, such as tumor immune escape. Many human tumors express tumor-associated antigens recognised by T cells and capable of inducing an immune response.

Cancer

Cancers may also be treated where there is no indication of a T-cell dysfunctional disorder, but the use of an antigen-binding molecule, cell or composition according to the present invention stimulates proliferation and expansion of T cells (particularly effector T cells) and allows the subject to mount an effective immune response.

The cancer to be treated/prevented in accordance with the invention described herein may be any unwanted cell proliferation (or any disease manifesting itself by unwanted cell proliferation), neoplasm or tumor. The cancer may be benign or malignant and may be primary or secondary (metastatic). A neoplasm or tumor may be any abnormal growth or proliferation of cells and may be located in any tissue. The cancer may be of tissues/cells derived from e.g, the adrenal gland, adrenal medulla, anus, appendix, bladder, blood, bone, bone marrow, brain, breast, cecum, central nervous system (including or excluding the brain), cerebellum, cervix, colon, duodenum, endometrium, epithelial cells (e.g. renal epithelia), gallbladder, oesophagus, glial cells, heart, ileum, jejunum, kidney, lacrimal glad, larynx, liver, lung, lymph, lymph node, lymphoblast, maxilla, mediastinum, mesentery, myometrium, nasopharynx, omentum, oral cavity, ovary, pancreas, parotid gland, peripheral nervous system, peritoneum, pleura, prostate, salivary gland, sigmoid colon, skin, small intestine, soft tissues, spleen, stomach, testis, thymus, thyroid gland, tongue, tonsil, trachea, uterus, vulva, white blood cells.

In some embodiments, the cancer to be treated may be a cancer of a tissue selected from the group consisting of colon, rectum, nasopharynx, cervix, oropharynx, stomach, liver, head and neck, oral cavity, oesophagus, lip, mouth, tongue, tonsil, nose, throat, salivary gland, sinus, pharynx, larynx, prostate, lung, bladder, skin, kidney, ovary or mesothelium.

Tumors to be treated may be nervous or non-nervous system tumors. Nervous system tumors may originate either in the central or peripheral nervous system, e.g. glioma, medulloblastoma, meningioma neurofibroma, ependymoma, Schwannoma, neurofibrosarcoma, astrocytoma and oligodendroglioma. Non-nervous system cancers/tumors may originate in any other non-nervous tissue, examples include melanoma, mesothelioma, lymphoma, myeloma, leukemia, Non-Hodgkin's lymphoma (NHL), Hodgkin's lymphoma, chronic myelogenous leukemia (CML), acute myeloid leukemia (AML), myelodysplastic syndrome (MDS), cutaneous T-cell lymphoma (CTCL), chronic lymphocytic leukemia (CLL), hepatoma, epidermoid carcinoma, prostate carcinoma, breast cancer, lung cancer, colon cancer, ovarian cancer, pancreatic cancer, thymic carcinoma, NSCLC, haematologic cancer and sarcoma.

In some embodiments, the cancer to be treated may be colon cancer, colon carcinoma, colorectal cancer, nasopharyngeal carcinoma, cervical carcinoma, oropharyngeal carcinoma, gastric carcinoma, hepatocellular carcinoma, head and neck cancer, head and neck squamous cell carcinoma (HNSCC), oral cancer, laryngeal cancer, prostate cancer, lung cancer, small cell lung cancer, non-small cell lung cancer, bladder cancer, urothelial carcinoma, melanoma, advanced melanoma, renal cell carcinoma, Ovarian cancer or mesothelioma.

In some embodiments the cancer to be treated/prevented in accordance with the present invention is a virus-associated cancer, e.g. an EBV-associated cancer or a HPV-associated cancer. “EBV associated” and “HPV associated” cancers may be a cancers which are caused or exacerbated by infection with the respective viruses, cancers for which infection is a risk factor and/or cancers for which infection is positively associated with onset, development, progression, severity of metastasis.

EBV-associated cancers which may be treated with cells produced by methods of the disclosure include nasopharyngeal carcinoma (NPC) and gastric carcinoma (GC).

HPV-associated medical conditions that may be treated with cells produced by methods of the disclosure include at least dysplasias of the genital area(s), cervical intraepithelial neoplasia, vulvar intraepithelial neoplasia, penile intraepithelial neoplasia, anal intraepithelial neoplasia, cervical cancer, anal cancer, vulvar cancer, vaginal cancer, penile cancer, genital cancers, oral papillomas, oropharyngeal cancer.

In some embodiments, the cancer to be treated in accordance with various aspects of the present disclosure is one or more of nasopharyngeal carcinoma (NPC: e.g. Epstein-Barr Virus (EBV)-positive NPC), cervical carcinoma (CC; e.g. human papillomavirus (HPV)-positive CC), oropharyngeal carcinoma (OPC; e.g. HPV-positive OPC), gastric carcinoma (GC; e.g. EBV-positive GC), hepatocellular carcinoma (HCC; e.g. Hepatitis B Virus (HBV)-positive HCC), lung cancer (e.g. non-small cell lung cancer (NSCLC)) and head and neck cancer (e.g. cancer originating from tissues of the lip, mouth, nose, sinuses, pharynx or larynx, e.g. head and neck squamous cell carcinoma (HNSCC)).

The treatment may be aimed at reducing the number of cells of the cancer, and/or reducing the size of a tumour, and/or inhibiting signalling mediated by an immune checkpoint protein (e.g. PD-1).

Administration of the antigen-binding molecules and compositions described herein may delay or prevent the onset of symptoms of the cancer. Administration of the antigen-binding molecules and compositions described herein may reduce the severity of symptoms of the cancer. Administration of the antigen-binding molecules and compositions described herein may delay or prevent the onset of invasion and/or metastasis. Administration of the antigen-binding molecules and compositions described herein reduce invasion and/or metastasis. Administration of the antigen-binding molecules and compositions described herein may decrease survival of cancer cells. Administration of the antigen-binding molecules and compositions described herein may increase survival of non-cancer cells and/or may increase survival of subjects.

Infection

An infection may be any infection or infectious disease, e.g. bacterial, viral, fungal, or parasitic infection. In some embodiments it may be particularly desirable to treat chronic/persistent infections, e.g. where such infections are associated with T cell dysfunction or T cell exhaustion.

It is well established that T cell exhaustion is a state of T cell dysfunction that arises during many chronic infections (including viral, bacterial and parasitic), as well as in cancer (Wherry Nature Immunology Vol. 12. No. B. p492-499, June 2011).

Examples of bacterial infections that may be treated include infection by Bacillus spp., Bordetella pertussis, Clostridium spp., Corynebacterium spp., Vibrio chloerae, Staphylococcus spp., Streptococcus spp. Escherichia, Klebsiella, Proteus, Yersinia, Ervina, Salmonella, Listeria sp, Helicobacter pylori, mycobacteria (e.g. Mycobacterium tuberculosis) and Pseudomonas aeruginosa. For example, the bacterial infection may be sepsis or tuberculosis.

Examples of viral infections that may be treated include infection by influenza virus, measles virus, hepatitis B virus (HBV), hepatitis C virus (HCV), human immunodeficiency virus (HIV), lymphocytic choriomeningitis virus (LCMV), Herpes simplex virus and human papilloma virus (HPV).

Examples of fungal infections that may be treated include infection by Alternaria sp. Aspergillus sp. Candida sp and Histoplasma sp. The fungal infection may be fungal sepsis or histoplasmosis.

Examples of parasitic infections that may be treated include infection by Plasmodium species (e.g. Plasmodium falciparum, Plasmodium yoeli, Plasmodium ovale, Plasmodium vivax, or Plasmodium chabaudi chabaudi). The parasitic infection may be a disease such as malaria, leishmaniasis and toxoplasmosis.

Methods of medical treatment may also involve in vivo, ex vivo, and adoptive immunotherapies, including those using autologous and/or heterologous cells or immortalised cell lines.

Adoptive Transfer

The antigen-binding molecules of the present invention are also useful in connection with methods comprising adoptive cell transfer (ACT). In particular, the antigen-binding molecules of the present invention are useful in methods for generating/expanding populations of immune cells in vitro or ex vivo, which may then be administered to subject. Methods for generating/expanding populations of immune cells may also be performed in vivo.

The present invention provides a method of treatment or prophylaxis comprising adoptive transfer of immune cells (e.g. T cells, effector T cells, virus-specific T cells. NK cells) produced (i.e. generated of expanded) according to the methods of the present invention. Adoptive cell transfer generally refers to a process by which immune cells are obtained from a subject, typically by drawing a blood sample from which the immune cells are isolated. The immune cells are then typically treated or altered in some way, optionally expanded, and then administered either to the same subject or to a different subject. The treatment is typically aimed at providing an immune cell population with certain desired characteristics to a subject, of increasing the frequency of immune cells with such characteristics in that subject.

The immune cells may be e.g. T cells, antigen-specific T cells (e.g. virus-specific T cells), antigen-specific CD4 T cells, antigen-specific CD8 T cells, effector memory CD4 T cells, effector memory CD8 T cells, central memory CD4 T cells, central memory CD8 T cells, cytotoxic CD8+ T cells (i.e. CTLs) NK cells or antigen-specific NK cells. The immune cells preferably express CD122 and CD132.

The cells may be antigen-specific immune cells, e.g. antigen-specific T cells. For example, the cells may be specific for a peptide/polypeptide of a virus, e.g. adenovirus, Epstein-Barr virus (EBV); cytomegalovirus (CMV), human papilloma virus (HPV), influenza virus, measles virus, hepatitis B virus (HBV), hepatitis C virus (HCV), human immunodeficiency virus (HIV), lymphocytic choriomeningitis virus (LCMV), or herpes simplex virus (HSV). Virus-specific immune cells may be an adenovirus-specific T cells (AdVSTs), Epstein-Barr virus-specific T cells (EBVSTs), cytomegalovius-specific T cells (CMVSTs), human papilloma virus-specific T cells (HPVSTs), influenza virus-specific T cells, measles virus-specific T cells, hepatitis B virus-specific T cells (HBVSTs) hepatitis C virus-specific T cells (HCVSTs), human immunodeficiency virus-specific T calls (HIVSTs), lymphocytic choriomeningitis virus-specific T cells (LCMVSTs), or herpes simplex virus-specific T cells (HSVSTs).

The cells may comprise/express a chimeric antigen receptor (CAR) or nucleic acid encoding a CAR. The cells may comprise/express a TGFβ decoy receptor, or nucleic acid encoding a TGFβ decoy receptor.

In some cases, the immune cells are derived from the patient that they are introduced to (autologous cell therapy). That is, cells may have been obtained from the patient, generated according to methods described herein, and then returned to the same patient. Methods disclosed herein may also be used in allogeneic cell therapy, in which cells obtained from a different individual are introduced into the patient.

Adoptive T cell transfer is described, for example, in Chia W K et al., Molecular Therapy (2014), 22(1); 132-139. Kalos and June 2013, Immunity 39(1): 49-60 and Cobbold et al., (2005) J. Exp. Med. 202; 379-386, which are hereby incorporated by reference in their entirety.

In the present invention, adoptive transfer is performed with the aim of introducing, or increasing the frequency of, Immune cells in a subject.

Accordingly, the present invention provides a method of treating or preventing a disease or condition in a subject, comprising:
(a) isolating PBMCs from a subject;
(b) generating or expanding e population of immune cells by culture in the presence of an antigen-binding molecule according to the present invention, and;
(c) administering the generated/expanded population of immune cells to a subject.

In some embodiments, the subject from which the PBMCs are isolated is the subject administered with the generated/expanded cells (i.e., adoptive transfer is of autologous cells). In some embodiments, the Subject from which the PBMCs are isolated is a different subject to the subject to which the generated/expanded cells are administered (i.e., adoptive transfer is of allogenic cells).

In some embodiments the method may comprise one or more of the following steps: taking a blood sample from a subject: isolating PBMCs from the blood sample: generating or expanding a population of immune cells by culture in the presence of an antigen-binding molecule according to the present invention; collecting the generated or expanded population of immune cells; mixing the generated or expanded population of immune cells with an adjuvant, diluent, or carrier; administering the generated or expanded population of immune cells or composition to a subject.

In some embodiments, the method may additionally comprise administering to a subject a therapeutically or prophylactically effective amount of an antigen-binding molecule according to the present invention.

The skilled person is able to determine appropriate reagents and procedures for adoptive transfer of immune cells generated or expanded according to the methods of the present invention for example by reference to Chia W K et al., Molecular Therapy (2014), 22(1): 132-139, Kalos and June 2013. Immunity 39(1): 49-60 and Cobbold et al. (2006) J. Exp. Med 202; 379-386.

Administration

Administration of an antigen-binding molecule or composition according to the invention is preferably in a “therapeutically effective” or “prophylactically effective” amount, this being sufficient to show benefit to the subject. The actual amount administered, and rate and time-course of administration, will depend on the nature and severity of the disease or disorder. Prescription of treatment, e.g. decisions on dosage etc., is within the responsibility of general practitioners and other medical doctors, and typically takes account of the disease/disorder to be treated, the condition of the individual subject, the site of delivery, the method of administration and other factors known to practitioners. Examples of the techniques and protocols mentioned above can be found in Remington's Pharmaceutical Sciences. 20th Edition, 2000, pub. Lippincott Williams & Wilkins. It will be appreciated that the articles of the present invention (e.g. the antigen-binding molecules or compositions of the invention) are administered in an amount sufficient to cause an increase in the number of (i.e. cause expansion a population of) cells expressing CD122 and CD132 (e.g. effector T cells. NK cells and/or CAR-T cells).

Administration of an antigen-binding molecule encompasses administration of a cell comprising/expressing the antigen-binding molecule.

Administration may be alone or in combination with other treatments, either simultaneously or sequentially dependent upon the disease/condition to be treated/prevented. The antigen-binding molecule or composition described herein and an additional agent, e.g. a therapeutic agent, may be administered simultaneously or sequentially. In some embodiments, the methods comprise additional therapeutic or prophylactic intervention, e.g. for the treatment/prevention of a cancer. In some embodiments, the therapeutic or prophylactic intervention is selected from chemotherapy, immunotherapy, radiotherapy, surgery, vaccination and/or hormone therapy.

Simultaneous administration refers to administration of the antigen-binding molecule, nucleic acid, vector, cell or composition and therapeutic agent together, for example as a pharmaceutical composition containing both agents (combined preparation), or immediately after each other and optionally via the same route of administration, e.g. to the same artery, vein or other blood vessel. Sequential administration refers to administration of one of the antigen-binding molecule/composition or therapeutic agent followed after a given time interval by separate administration of the other agent. It is not required that the two agents are administered by the same route, although this is the case in some embodiments. The time interval may be any time interval.

In some embodiments, the antigen-binding molecule is administered in combination with a chimeric antigen receptor (CAR) or a CAR-expressing cell (e.g. a CAR-T cell). In some embodiments, the antigen-binding molecule is administered in combination with a population of immune cells, e.g. a population of immune cells generated/expanded according to the methods described herein. In some embodiments, the antigen-binding molecule is administered in a method comprising adoptive cell transfer (ACT), as described herein.

In some embodiments, the antigen-binding molecule is administered in combination with an agent capable of inhibiting signalling mediated by an immune checkpoint protein, immune checkpoint proteins are sometimes referred to as immune checkpoint inhibitors.

Immure checkpoint proteins help keep immune responses in check and thereby protect against autoimmunity, but can also act to inhibit desirable effector immune cell activity such as killing of cancer cells by T cells, or killing of cells infected with a pathogen (particularly in instances of chronic infection). Inhibition of signalling mediated by immune checkpoint proteins is desirable in such settings as it releases effector immune cells from immune checkpoint protein-mediated inhibition, thereby promoting effector immune responses.

Agents capable of inhibiting signalling mediated by a given immune checkpoint protein include, e.g. agents capable of binding to the immune checkpoint protein or a ligand for the immune checkpoint protein and inhibiting signalling mediated by the immune checkpoint protein; agents capable of reducing gene/protein expression of the immune checkpoint protein or a ligand for the immune checkpoint protein (e.g. through inhibiting transcription of the gene(s) encoding the immune checkpoint protein/ligand, inhibiting post-transcriptional processing of RNA encoding the immune checkpoint protein/ligand, reducing stability of RNA encoding the immune checkpoint protein/ligand, promoting degradation of RNA encoding the immune checkpoint protein/ligand, inhibiting post-translational processing of the immune checkpoint protein/ligand, reducing stability the immune checkpoint protein/ligand, or promoting degradation of the immune checkpoint protein/ligand), and small molecule inhibitors.

In some embodiments the immune checkpoint protein is PD-1, CTLA-4, LAG-3. TIM-3, VISTA, TIGIT or BTLA. In some embodiments the immune checkpoint protein is selected from PD-1, CTLA-4, LAG-3 and TIM-3.

In some embodiments, the antigen-binding molecule of the present invention is administered in combination with an agent capable of inhibiting signalling mediated by PD-1. The agent capable of inhibiting signalling mediated by PD-1 may be a PD-1-targeted agent, or an agent targeted against a ligand for PD-1 such as PD-L1 or PD-L2. In some embodiments, the agent capable of inhibiting signalling mediated by PD-1 may e.g. be an antibody capable of binding to PD-1. PD-L1 or PD-L2 and inhibiting PD-1-mediated signalling. In some embodiments the agent capable of inhibiting signalling mediated by PD-1 is an antibody/fragment described in WO2016/068801 or WO2016/111645, both of which are hereby incorporated by reference in their entirety.

In some embodiments, the antigen-binding molecule of the present invention is administered in combination with an agent capable of inhibiting signalling mediated by CTLA-4 The agent capable of inhibiting signalling mediated by CTLA-4 may be a CTLA-4-targeted agent, or an agent targeted against a ligand for CTLA-4 such as CD80 or CD66. In some embodiments, the agent capable of inhibiting signalling mediated by CTLA-4 may e.g. be an antibody capable of binding to CTLA-4, CD80 of CD86 and inhibiting CTLA-4-mediated signalling. In some embodiments the agent capable of inhibiting signalling mediated by CTLA-4 is an antibody/fragment described in WO2017/194265, which is hereby incorporated by reference in its entirety.

In some embodiments, the antigen-binding molecule of the present invention is administered in combination with an agent capable of inhibiting signalling mediated by LAG-3. The agent capable of inhibiting signalling mediated by LAG-3 may be a LAG-3-targeted agent, or an agent targeted against a ligand for LAG-3 such as MHC class II. In some embodiments, the agent capable of inhibiting signalling mediated by PD-1 may e.g. be an antibody capable of binding to LAG-3 or MHC Class II and inhibiting LAG-3-mediated signalling. In some embodiments the agent capable of inhibiting signalling mediated by LAG-3 is an antibody/fragment described in WO2017/149143, which is hereby incorporated by reference in its entirety.

In some embodiments, the antigen-binding molecule of the present invention is administered in combination with an agent capable of inhibiting signalling mediated by TIM-3. The agent capable of inhibiting signalling mediated by TIM-3 may be a TIM-3-targeted agent, or an agent targeted against a ligand for TIM-3 such as Galectin 9. In some embodiments, the agent capable of inhibiting signalling mediated by TIM-3 may e.g. be an antibody capable of binding to TIM-3 or Galectin 9 and inhibiting TIM-3-mediated signalling. In some embodiments the agent capable of inhibiting signalling mediated by PD-1 is an antibody/fragment described in WO2016/068602 or WO2016/068803, both of which are hereby incorporated by reference in their entirety.

Chemotherapy and radiotherapy respectively refer to treatment of a cancer with a drug or with ionising radiation (e.g. radiotherapy using X-rays of γ-rays). The drug may be a chemical entity, e.g. small molecule pharmaceutical, antibiotic. DNA intercalator, protein inhibitor (e.g. kinase inhibitor), or a biological agent, e.g. antibody, antibody fragment, aptamer, nucleic acid (e.g. DNA, RNA), peptide, polypeptide, of protein. The drug may be formulated as a pharmaceutical composition or medicament.

The formulation may comprise one or more drugs (e.g. one or more active agents) together with one or more pharmaceutically acceptable diluents, excipients or carriers.

A treatment may involve administration of more than one drug. A drug may be administered alone or in combination with other treatments, either simultaneously or sequentially dependent upon the condition to be treated. For example, the chemotherapy may be a co-therapy involving administration of two drugs, one or more of which may be intended to treat the cancer.

The chemotherapy may be administered by one or more routes of administration, e.g. parenteral, intravenous injection, oral, subcutaneous, intradermal or intratumoral.

The chemotherapy may be administered according to a treatment regime. The treatment regime may be a pre-determined timetable, plan, scheme or schedule of chemotherapy administration which may be prepared by a physician or medical practitioner and may be tailored to suit the patient requiring treatment.

The treatment regime may indicate one or more of: the type of chemotherapy to administer to the patient; the dose of each drug or radiation: the time interval between administrations; the length of each treatment; the number and nature of any treatment holidays, if any etc. For a co-therapy a single treatment regime may be provided which indicates how each drug is to be administered.

Chemotherapeutic drugs and biologics may be selected from: alkylating agents such as cisplatin, carboplatin, mechlorethamine, cyclophosphamide, chlorambucil, ifosfamide, purine or pyrimidine anti-metabolites such as azathiopurine or mercaptopurine; alkaloids and terpenoids, such as vinca alkaloids (e.g. vincristine, vinblastine, vinorelbine, vindesine), podophyllotoxin, etoposide, teniposide, taxanes such as paclitaxel (Taxol™), docetaxel; topoisomerase inhibitors such as the type I topoisomerase inhibitors camptothecins irinotecan and topolecan, or the type II topoisomerase inhibitors amsacrine, etoposide, etoposide phosphate, teniposide; antitumor antibiotics (e.g. anthracycline antibiotics) such as dactinomycin, doxorubicin (Adriamycin™), epirubicin, bleomycin, rapamycin; antibody based agents, such as anti-PD-1 antibodies, anti-PD-L1 antibodies, anti-TIM-3 antibodies, anti-CTLA-4, anti-4-1BB, anti-GITR, anti-CD27, anti-BLTA, anti-OX43, anti-VEGF, anti-TNFα, anti-IL-2, antiGplIb/IIIa, anti-CD-52, anti-CD20, anti-RSV, anti-HER2/neu(erbB2), anti-TNF receptor, anti-EGFR antibodies, monoclonal antibodies or antibody fragments, examples include: cetuximab, panitumumab, infliximab, basiliximab, bevacizumab (Avastin®), abciximab, daclizumab, gemtuzumab, alemtuzumab, rituximab (Mabthera®), palivizumab, trastuzumab, etanercept, adalimumab, nimotuzumab; EGFR inhibitors such as erlotinib, cetuximab and gefitinib, anti-angiogenic agents such as bevacizumab (Avastin®); cancer vaccines such as Sipuleucel-T (Provenge®).

Further chemotherapeutic drugs may be selected from: 13-cis-Retinoic Acid, 2-Chlorodeoxyadenosine, 5-Azacitidine 5-Fluorouracil, 6-Mercaptopurine, 6-Thioguanine, Abraxane, Accutane®, Actinomycin-D Adriamycin®, Adrucil, Afinitor®, Agrylin®, Ala-Cort®, Aldesleukin, Alemtuzumab, ALIMTA, Alitretinoin. Alkaban-AQ®, Alkeran®, All-transretinoic Acid, Alpha Interferon, Altretamine, Amethopterin, Amifostine, Aminoglutethimide, Anagrelide, Anandron®, Anastrozole, Arabinosylcytosine, Aranesp®, Aredia®, Arimidex®, Aromasin®, Arranon, Arsenic Trioxide, Asparaginase, ATRA Avastin®, Azacitidine, BCG, BCNU, Bendamustine, Bevacizumab, Bexarotene, BEXXAR®, Bicalutamide, BiCNU, Blenoxane®, Bleomycin, Bortezomib, Busulfan, Busulfex, Calcium Leucovorin, Campath®, Camptosar®, Camptothecin-11. Capecitabine, Carac™, Carboplatin, Carmustine, Casodex®, CC-6013, CCI-779. CCNU, CDDP, CeeNU, Cerubidine, Cetuximab, Chlorambucil, Cisplatin, Citrovorum Factor, Cladribine, Cortisone, Cosmegen®, CPT-11, Cyclophosphamide, Cytadren®, Cytarabine Cytosar-UR, Cytoxan®, Dacogen, Dactinomycin, Darbepoetin Alfa, Dasatinib, Daunomycin, Daunorubicin, Daunorubicin Hydrochloride, Daunorubicin Liposomal, DaunoXome®, Decadron, Decitabine, Della-Cortef), Deltasone®, Denileukin, Diftitox, DepoCyt™, Dexamethasone, Dexamethasone Acetate, Dexamethasone Sodium Phosphate, Dexasone, Dexrazoxane, DHAD, DIC, Diodex, Docetaxel, Doxil®, Doxorubicin, Doxorubicin Liposomal, Droxia™, DTIC, DTIC-Dome, Duralone®, Eligard™, Ellence™, Eloxatin™, Elspar®, Emcyt®, Epirubicin, Epoetin Alfa, Erbitux, Erlotinib, Erwinia L-asparaginase, Estramustine, Ethyol Etopophos®, Etoposide, Eloposide Phosphate, Eulexin®, Everolimus, Evista®, Exemestane, Faslodex®, Femara®, Filgrastim, Floxuridine, Fludara®, Fludarabine, Fluoroplex®, Fluorouracil, Fluoxymesterone, Flutamide, Folinic Acid, FUDR®, Fulvestrant, Gefitinib, Gemcitabine, Gemtuzumab ozogamicin, Gleevec™, Gliadel® Wafer, Goserelin, Granulocyte—Colony Stimulating Factor, Granulocyte Macrophage Colony Stimulating Factor, Herceptin®, Hexadrol, Hexalen®, Hexamethylmelamine, HMM, Hycamlin®, Hydrea®, Hydrocort Acetate®, Hydrocortisone, Hydrocortisone Sodium Phosphate, Hydrocortisone Sodium Succinate, Hydrocortone Phosphate, Hydroxyurea, Ibritumomab. Ibritumomab Tiuxetan, Idamycin®, Idarubicin, Ifex®, IFN-alpha, Ifosfamide, IL-11, IL-2, Imatinib mesylate, imidazole Carboxamide, Interferon alia, Interferon Alfa-2b (PEG Conjugate), Interleukin-2, Interleukin-11, Intron A® (interferon alfa-26), Iressa®, Irinotecan, Isotretinoin, Ixabepilone, Ixempra™, Kidrolase, Lanacort®, Lapalinib, L-asparaginase, LCR, Lenalidomide, Letrozole, Leucovorin, Leukeran, Leukine™, Leuprolide, Laurocristine, Leustatin™, Liposomal Ara-C, Liquid Pred®, Lomustine, L-PAM, L-Sarcolysin, Lupron®, Lupron Depot®, Matulane®, Maxidex, Mechlorethamine, Mechlorethamine Hydrochloride, Medralone®, Medrol®, Megace®, Megestrol, Megestrol Acetate, Melphalan, Mercaptopurine, Mesna, Mesnex™, Methotrexate, Methotrexate Sodium, Methylprednisolone, Meticorten®, Mitomycin, Mitomycin-C, Mitoxantrone, M-Prednisol®, MTC, MTX, Mustargen®, Mustine, Mutamycin®, Myleran®, Mylocel™, Mylotarg®, Navelbine®, Nelarabine, Necsar®, Neulasta™, Neumega®, Neupogen®, Nexavar®, Nilandron®, Nilutamide, Nipent®, Nitrogen Mustard, Novaldex®, Novantrone®, Octreotide, Ochreotide acetate, Oncospar®, Oncovin®, Ontak®, Onxal™, Oprevelkin, Orapred®, Orasone®, Oxaliplatin, Paclitaxel, Paclitaxel Protein-bound, Pamidronate, Panitumumab, Panretin®, Paraplatin®, Pediapred®, PEG Interferon, Pegaspargase, Pegfilgrastim, PEG-INTRON™, PEG-L-asparaginase, PEMETREXED, Pentostatin, Phenylalanine Mustard, Platinol®, Platinol-AQ®, Prednisolone, Prednisone, Prelone, Procarbazine, PROCRIT®, Proleukin®, Prolifeprospan 20 with Carmustine Implant Purinethol®, Raloxifene, Revlimid®, Rheumatrex®, Rituxan®, Rituximab, Roferon-A® (Interferon Alfa-2a), Rubex®, Rubidomycin hydrochloride, Sandostatin® Sandostatin LAR®, Sargramostim, Solu-Cortef®, Solu-Medrol®, Sorafenib, SPRYCEL™, STI-571, Streptozocin, SU11248, Sunitinib, Sutent®, Tamoxifen, Tarceva®, Targretin®, Taxol, Taxotere®, Temodar, Temozolode, Temsirolimus, Teniposide, TESPA, Thalidomide, Thalomid®, TheraCys®, Thioguanine, Thioguanine Tabloid®, Thiophosphoamide, Thioplex®, Thiotepa, TICE®, Toposar®, Topolecan, Toremifene, Torisel®, Tositumomab, Trastuzumab, Treanda®, Tretinoin, Trexall™, Trisenox®, TSPA, TYKERB®, VCR, Vectibix™, Velban®, Velcade®, VePesid®, Vesanoid®, Viadur™, Vidaza®, Vinblastine, Vinblastine Sulfate, Vincasar Pfs®, Vincristine, Vinorelbine, Vinorelbine tartrate, VLB, VM-26, Vorinostat, VP-16, Vumon®, Xeloda®, Zanosar®, Zevalin™, Zinecard®, Zoladex®, Zoledronic acid, Zolinza, Zometa®.

Multiple doses of the antigen-binding molecule or composition may be provided. One or more, or each, of the doses may be accompanied by simultaneous or sequential administration of another therapeutic agent.

Multiple doses may be separated by a predetermined time interval, which may be selected to be one of 1, 2, 3, 4, 6, 6, 7, 8, 9, 10, 11, 12, 13, 14, 15, 16, 17, 18, 19, 20, 21, 22, 23, 24, 25, 26, 27, 28, 29, 30, or 31 days, or 1, 2, 3, 4, 5, or 6 months. By way of example, doses may be given once every 7, 14, 21 or 28 days (plus or minus 3, 2, or 1 days).

In some embodiments, the antigen-binding molecule may be conjugated to a drug moiety or a detectable moiety.

Methods of Detection

The antigen-binding molecules described herein may be used in methods that involve detection of CD122 and/or CD132, or cells expressing CD122 and/or CD132 (e.g. at the cell surface). The methods may be in vitro or in vivo methods. Such methods may involve detection of the bound complex of the antigen-binding molecule and CD122 and/or CD132, or cells expressing CD122 and/or CD132.

As such, a method is provided, the method comprising contacting a sample containing, or suspected to contain, CD122 and/or CD132 or cells expressing CD122 and/or CD132, and detecting the formation of a complex of the antigen-binding molecule and CD122 and/or CD132 or cells expressing CD122 and/or CD132.

Suitable method formats are well known in the art, including immunoassays such as sandwich assays, e.g. ELISA. The methods may involve labelling the antigen-binding molecule, or target(s), or both, with a detectable moiety, e.g. a delectable moiety as described hereinabove. In some embodiment the detectable moiety is a fluorescent label, a luminescent label, an immuno-detectable label or a radio-label. In some embodiments, the detectable moiety may be selected from: a radio-nucleotide, positron-emitting radionuclide (e.g. for positron emission tomography (PET)), MRI contrast agent or fluorescent label Analysis in vitro or in vivo may involve analysis by positron emission tomography (PET), magnetic resonance imaging (MRI), or fluorescence imaging, e.g. by detection of appropriately labelled species.

Methods of this kind may provide the basis of methods for the diagnostic and/or prognostic evaluation of a disease or condition. Such methods may be performed in vitro on a patient sample, or following processing of a patient sample. Once the sample is collected, the patient is not required to be present for the in vitro method to be performed, and therefore the method may be one which is not practised on the human or animal body.

In some embodiments the methods may involve detecting or quantifying CD122 and/or CD132, or cells expressing CD122 and/or CD132, e.g. in a patient sample. Where the method comprises quantifying the relevant factor, the method may further comprise comparing the determined amount against a standard or reference value as part of the diagnostic or prognostic evaluation. Other diagnostic/prognostic tests may be used in conjunction with those described herein to enhance the accuracy of the diagnosis of prognosis or to confirm a result obtained by using the tests described herein.

A sample may be taken from any tissue or bodily fluid. The sample may comprise or may be derived from: a quantity of blood; a quantity of serum derived from the individual's blood which may comprise the fluid portion of the blood obtained after removal of the fibrin clot and blood cells; a tissue sample or biopsy; pleural fluid, cerebrospinal fluid (CSF); or cells isolated from sail individual. In some embodiments, the sample may be obtained or derived from a tissue or tissues which are affected by the disease/condition (e.g. tissue or tissues in which symptoms of the disease manifest, or which are involved in the pathogenesis of the disease/condition).

Subjects

The subject to be treated in accordance with aspects the invention described herein may be any animal or human. The subject is preferably mammalian, more preferably human. The subject may be a non-human mammal, but is more preferably human. The subject may be male or female. The subject may be a patient. A subject may have been diagnosed with a disease or condition requiring treatment (e.g. a cancer), may be suspected of having such a disease/condition, or may be at risk of developing/contracting such a disease/condition.

In embodiments according to the present invention the subject is preferably a human subject. In some embodiments, the subject to be treated according to a therapeutic or prophylactic method of the invention herein is a subject having, or at risk of developing, a cancer. In embodiments according to the present invention, a subject may be selected for treatment according to the methods based on characterisation for certain markers of such disease/condition.

Kits

The present invention also provides a kit of parts. In some embodiments the kit may have at least one container having a predetermined quantity of an antigen-binding molecule, nucleic acid, expression vector, CAR, composition or calls described herein.

The kit may provide the antigen-binding molecule, nucleic acid, expression vector, CAR, composition or cells together with instructions for administration to a patient in order to treat a specified disease/condition.

In some embodiments, the kit may comprise materials for producing antigen-binding molecule or composition described herein.

The kit may additionally instructions for administration to a patient in order to treat a specified disease/condition. In some embodiments, the kit may comprise materials and/or instructions for producing an antigen-binding molecule, nucleic acid, expression vector, CAR, cell or composition described herein.

In some embodiments the kit may further comprise at least one container having a predetermined quantity of another therapeutic agent (e.g. anti-infective agent or chemotherapy agent). In such embodiments, the kit may also comprise a second medicament or pharmaceutical composition such that the two medicaments or pharmaceutical compositions may be administered simultaneously or separately such that they provide a combined treatment for the specific disease or condition.

Sequence Identity

Pairwise and multiple sequence alignment for the purposes of determining percent identity between two or more amino acid or nucleic acid sequences can be achieved in various ways known to a person of skill in the art, for instance, using publicly available computer software such as ClustalOmega (Söding, J. 2005, Bioinformatics 21, 951-960), T-coffee (Notredame et al. 2000. J. Mol. Biol. (2000) 302, 205-217) Kalign (Lassmann and Sonnhammer 2005, BMC Bioinformatics, 6(298)) and MAFFT (Kaloh and Standley 2013, Molecular Biology and Evolution, 30(4) 772 780 software. When using such software, the default parameters, e.g. for gap penalty and extension penalty, are preferably used.

Sequences

| SEQ | | |
|---|---|---|
| ID | | |
| NO: | DESCRIPTION | SEQUENCE |
| 1 | Anti CD122 heavy chain; | EVQLVQSGAEVKKPGSSVKVSCKASGYTFTNYYMHWVRQAPGQGLEWMGAIMPSRG |
| | clones P2C4, P2C4_A4, | GTSYPQKFQGRVTMTGDTSTSTVYMELSSLRSEDTAVYYCARGEYYYDSSGYYYWGQ |
| | P2C4_B1, P2C4_B5, | GTLVTVSS |
| | P2C4_C4, P2C4_C7, | |
| | P2C4_010, P2C4_E6, | |
| | P2C4_E7, P2C4_F6 | |
| 2 | Anti CD122 heavy chain | EVQLVQSGTEVKKPGASVKVSCKASGYTFTTYAMHWWVRQAPGQSLEWMGWINTGNG |
| | clone P2H7 | NTKYSQNFQGRVTMTRDTSISTAYMELSRLRSQDTAVYYCARDLGQLERLYFWGQGTL |
| | | VTVSS |
| 3 | Anti CD122 heavy chain | HVQLVETGGGLVQPGGSLRLSCAASGFTFSSYAMSWVRQAPGKGLEWVSAISGSGGS |
| | clone P2D12 | TYYADSVKGRFTISRQNSKNTLYLQMNSLRAEDTAVYYCARDLGDYWGQGTLVTVSS |
| 4 | Anti CD122 heavy chain | QVQLQQWGAGLLKPSETLSLTCAVYGGSFSGYYWSWIRQPPGKGLEWIGEINHSGSTN |
| | clone P1G11 | YNPSLKSRVTISVDISKNQFSLKLSSVTAADTAVYYCARSSSGDAFDIWGQGTMVTVSS |
| 5 | Anti CD122 beav | EVQLVQSGAEVKKPGSSVKVSCKASGYTFTNYYMHWVRQAPGQGLEWMGAIMPSRG |
| | clone P2C4_A9 | GTSYPQKFQGRVTMTGDYSTSYVYMELSSLRSEDTAVYYCARGEYYYDSSGYYNWGQ |
| | | GTLVTVSS |
| 6 | Anti CD122 heavy chain | EVQLVQSGAEVKKPGSSYKVSCKASGYTFTNYYIHWVRQAPGQGLEWMGAIMPSRGG |
| | clones P2C4_B6, P2C4_E9 | TSYPQKFQGRVTMTGDTSTSTVYMELSSLRSEDTAVYYCARGEYYYDSSGYYYWGQG |
| | | TLVTVSS |
| 7 | Anti CD122 heavy chain | EVQLVQSGAEVKKPGSSVKVSCKASGYTFTNYYMHWVRQPPGQGLEWMGAIMPSRG |
| | clone P2C4_E9 | GTSYPQKFQGRVTMTGDTSTSTVYMELSSLRSEDTAVYYCARGEYYYDSSGYYYWGQ |
| | | GTLVTVSS |
| 8 | Anti CD122 heavy chain, | EVQLVQSGAEVKKPGSTVKVSCKASGYTFTNYYMHWVRQAPGQGLEWMGAIMPSRG |
| | clone P2C4_B12 | GTSYPQKFQGRVTMTGDTSTSTVYMELSSLRSEDTAVYYCARGEYYYDSSGYYYWGQ |
| | | GTLVTVSS |
| 9 | Anti CD122 heavy chain, | EVQLVQSGAEVKKPGSSVKVSCKASGYTFTNYYMHWVRGAPGQGLEWMGAIMPSRG |
| | clone P2C4_C1 | GTSYPQKFQGRVTMTGDTSTSTVYMELSSLRSEDTAVYYCARGEYYYDSSGYYYWGQ |
| | | GTPVTVSS |
| 10 | Anti CD122 heavy chain, | EVQLVQSGAEVKKPGSSVKVSCKASGYTFTNYYMHWVRQAPGQGLEWMGAIMPSRG |
| | clone P2C4_C12 | GTSYPQKFQGRVTMTGDTSTSTVYMELSNLRSEDTAVYYCARGEYYYDSSGYYYWGQ |
| | | GTLVTVSN |
| 11 | Anti CD122 heavy chain, | EVQLVQSGAEVKEPGSSVKVSCKASGYTFTNYYVHWVRQAPGQGLEWMGAIMPSRG |
| | clone P2C4_E2 | GTSYPQKFQGRVTMTGDISTSTVYMELSSLRSEDTAVYYCARGEYYYDSSGYYYWGQ |
| | | GTLVTVSS |
| 12 | Anti CD122 heavy chain, | EVQLVQSGAEVKKPGSSVKVSCKASGYTFTNYYIHWVRQAPGQGLEWMGAIMPSRGG |
| | clone P2C4_E3 | TSYPQKFQGRVTMTGDTSTSTVYMELNSLRSEDTAVYYCARGEYYYDSSGYYYWGQG |
| | | TLVTVSS |
| 13 | Anti CD122 heavy chain, | EVQLVQSGAEVKKPGSSYKVSCKASGYTFTNYYMHWVRQAPGQGLEWMGAIMPSRG |
| | clone P2C4_E8 | GTSYPQKFQGRVTMTGQTSTSTVYMELSSLRSEDTAVYYCARGEYYYDSSQYYYWGP |
| | | GTLVTVSS |
| 14 | Anti CD122 heavy chain, | EVQLVQSGAEVKKPGSSVKVSCKASGYTFTNYYMHWVRQAPGQGLEWMGAIMPSRG |
| | clone P2C4_F11 | GTSYPQKFQGRVTMTGDTSTSTVYMELSSLRSEDTAMYYCARGEYYYDSSGYYYWGQ |
| | | GTLVTVSS |
| 15 | Anti CD122 heavy chain, | EVQLVQSGAEVKKPGSSVKVSCKASGYTFTNYYMHWVRQAPGQGLEWMGAIMPSRG |
| | clone P2C4_G2 | GTSYPQKFQGRVTMTGDTSTSTVYMELSSLRTEDTAVYYCARGEYYYDSSGYYYWGQ |
| | | GTLVTVSS |
| 16 | Anti CD122 heavy chain, | EVQLYQSGAEVKKPGSSYKVSCKASGYTFTNYYMHWVRQAPGQGLEWMGAIMPSRG |
| | clone P2C4_G11 | GTSYPQKFQGRVTMTGDTSTSTVYMELSNLRSEDTAVYYCARGEYYYDSSGYYYWGQ |
| | | GTLVTVSS |
| 17 | Anti CD122 heavy chain, | EVQLVQSGAEVKKPGSSVKVSCKASGYTFTNYYMHWVRQAPGQGLEWMGAIMPSRG |
| | clone P2C4_H1 | GTSYPQKFQGRVTMTGQTSTSTVYMELSSLRSEDTAVYYCARGEYYYDSSGYYYWGQ |
| | | GTLVNVSS |
| 18 | Anti CD122 heavy chain, | EVQLVQSGAEVKKPGSSVKYSCKASGYTFSNYYMHWVRGAPGQGLEWIGAIMPSRGG |
| | clone P2C4_H2 | TSYPQKFQGRVTMTGQTSTSTVYMELSSLRSEDTAVYYCARGEYYYDSSGYYYWGQG |
| | | TLVTVSS |
| 19 | Anti CD122 heavy chain, | EVQLVQSGAEVKKPGSSVKVSCKATGYTFTNYYMHWVRQAPGQGLEWMGAIMPSRG |
| | clone P2C4_H3 | GTSYPQKFQGRVTMTGDTSTSTVYMELSSLRSEDTAVYYCARGEYYYDSSGYYYWGQ |
| | | GTLVTVSS |
| 20 | Anti CD122 heavy chain, | EVQLVQSGAEVKKPGSSVKVSCKASGYTFTNYYMHWVRQAPGQGLEWMGAIMPSRG |
| | clone P2C4_C1D10 | GTSYPQKFQGRVTMTGDTSTSTVYMELSSLRSEDTAVYYCARGEYYYDSSGYYYWGQ |
| | | GTPVTVSS |
| 21 | Anti CD122 heavy chain, | EVQLVQSGAEVKKPGASVKVSCKASGYTFTNYYMHWVRQAPGQGLEWMGAIMPSRG |
| | clone P2C4_FW2 | GTSYPQKFQGRVTITADKSTSTAYMELSSLRSEDTAVYYCARGEYYYDSSGYYYWGQG |
| | | TLVTVSS |
| 22 | Anti CD122 heavy chain, | EVQLVQSGGGVVQPGRSLRLSCAASGFTFSSYAMHWVRQAPGKGLEWVAVISYDGSN |
| | clone P1E7 | KYYADSVKGRFTISRQNSKNTLYLQMNSLRAEDTAVYYCARDLGYSSSWYYYYYGMDV |
| | | WGQGTTVTVSS |
| 23 | Anti CD122 heavy chain, | QVQLQESGPGLVKPSETLSLTCTVSGVSISSRSDHWGWVRQPPGKGLEWIGSISYSGS |
| | clone P1B10 | TYYNPSLKSRVTISVDTSKNQLSLKLSSVTAADTAVYYCARESHPAAALVGWGQGTLVT |
| | | VSS |
| 24 | Anti CD122 heavy chain, | EVQLVQSGGGLVQPGGSLRLSCAASGFTFSSYAMSWYRQAPGKGLEWVSAISGSGGS |
| | clone P1F3 | THYADSVKGRFTISRQNSKNTLYLQMNSLRAEDTAVYYCATPAFWGQGTLVTVSS |
| 25 | Anti CD122 heavy chain, | QVQLQQWGPGLVKPSETLSLTCTVSGGSISSYYWSWIRQPPGKGLEWIGEINKSGSTN |
| | clone P1D10 | YNPSLKSRVTISVDTSKNQFSLKLSSVTAADTAVYYCAGGSNLDWFDPWGQGTLVTVSS |
| 26 | Anti CD122 heavy chain, | QVQLQQWGAGLLKPSETLSLTCAVYGGSFSGYYWSWIRQPPGKGLEWIGEINHSGSTN |
| | clone P1E1 | YNPSLKSRVTISVDTSKNQFSLELSSVTAADTAVYYCARADRREGELRYWGQGTLVTVSS |
| 27 | Anti CD122 heavy chain, | QVQLVQSGGGLVKPGGSLRLSCAASGFTFSSYDLHWVRQVPGKGLEWVSLISYDGSNK |
| | clone P2B11 | YYADSVKGRFTISRDNAENSLYLQMNSLRAEDTAVYYCAREPITGTSDLFDYWGQGTLV |
| | | TVSS |
| 28 | Anti CD122 heavy chain, | QVQLQESGPGLVKPSGTLSLTCAVSGGSISSSNWWSWVRQPPGKGLEWIGEIYHSGST |
| | clone P2C9 | NYNPSLKSRVTISVDKSKNQFSLKLSSYTAADTAVYYCVREGGLREEHWGQGTLVTVSS |
| 29 | Anti CD122 heavy chain, | QVQLQQWGAGLLKPSETLSLTCAVYGGSFSGYYWSWIRQPPGKGLEWIGEINHSGSTN |
| | clone P2C10 | YNPSLKSRVTISVDTSKNQFSLKLSSVTAADTAVYYCARGTDTAMADYWGQGTLVTVSS |
| 30 | Anti CD122 heavy chain, | QVQLQQSGPGLVKPSQTLSLTCAISGDSVSGNSATWWWIRQSPSRGLEWLGRTYYRSK |
| | clone P2C14 | WNHDYAESVKSRITINPDTSKNQFSLQLNSVTPEDIAVYYCARDSKSAFDIWGQGTMVT |
| | | VSS |
| 31 | Anti CD122 heavy chain, | QLQLQESGPGLVKPSETLSLTCSVFGVSITSGSWWSWVRQSPGKELEWIGEIYHNGNT |
| | clone P2E6 | NYNPSLKSRVTISVDTSKNQFSLKLSSVTAADTAVYYCVSGFDYWGQGTLVT SS |
| 32 | Anti CD122 heavy chain, | QVQLQESGPGLVKPSETLSLTCTVSGVSISSRSDHWGWVRQPPGKGLEWIGSISYSGS |
| | clone P2E11 | TYYNPSLKSRVTISVDTSKNQLSLKLSSVTAADTAVYYCARESHPAAALVGWGQGTLVT |
| | | VSS |
| 33 | Anti CD122 heavy chain, | EVQLVQSGAFVKKPGSSYKVSCKASGYTFTSYGISWVRQAPGQGLEWMGWISAYNGN |
| | clone P2F9 | TNYACKLQGRVTMTTDTSTSTAYMELSSLRSEDTAVYYCARAPDYGDSSNYYYYYMDV |
| | | WGKGTTVTVSS |
| 34 | Anti CD122 heavy chain, | QVQLVQSGAEVKKPGASVKYSCKASGYTFTSYGISWVRQAPGQGLEWMGWISAYNGN |
| | clone P2F10 | TNYAQKLQGRVTMTTDTSTSTAYMELRSLRSDDTAVYYCARDTSGDYSSGWYLGVPFD |
| | | YWGQGTLVTVSS |
| 35 | Anti CD122 light chain, | QSALTQPASVSGSPGQSIAISCTGTSSDIGHYDFVSWYQQHPGTAPKLIIYDINNRPS |
| | clones P2C4, | GISNRFSGSKSQNMASLTISGLQPEDEADYYCSAYTSSDTLVFGGGTKLT |
| | P2C4_A9 | |
| 36 | Anti CD122 light chain, | DIQMTQSPSTLSASVGDRVTLSCRAGQAISSWLAWYQQKPGKAPKLLIYKASNLESGVP |
| | clone P2H7 | SRFSGGGSGAEFTLTISSLQPDQFATYYCQQYQSYPYTFGQGTKLEIR |
| 37 | Anti CD122 light chain, | DIQLTQSPSSLSASVGDRVTITCQASQDIGNYLNWYQLKPGKAPKLLIYDASNLETGVAS |
| | clone P2D12 | RFSGSGSGTDFTFTISSLQPEDIATYYCLQLYDYPLTFGGGTKVEIK |
| 38 | Anti CD122 light chain, | NFMLTQPHSVSESPGKTVTISCTRSSGSIASNYVQWYQQRPGSSPTTVIFDDNQRPTGV |
| | clone P1G11 | PDRFSAAIDTSSSSASLTISGLTAEDEADYYCQSSHSTAVVFGGGTKLTVL |
| 39 | Anti CD122 light chain, | QSALTQPASVSGSPGQSIAISCTGTSSDIGDYDFVSWYQQHPGTAPKLIIYDINNRPSGIS |
| | clones, P2C4_A4, P2C4_C1 | NRFSGSKSDNMASLTISGLQPEDEADYYCSAYTSSDTLVFGGGTKET |
| 40 | Anti CD122 light chain, | QSALTQPASVSGSPGQSIAISCTGTSSDIGHYDFVSWYCQHPGTAPKLIIYQNNNRPSGI |
| | clone P2C4_B1 | SNRPSGSKSDNMASLTISGLQPEDEADYYCSAYYSSDTLVFGGGTKLT |
| 41 | Anti CD122 light chain, | QSALTQPASVSGSPGQSITISCTGTSSDIGHYDFVSWYQQHPGTAPKLIIYDNNNRPSGIS |
| | clone P2C4_B5 | NRFSGSKSDNMASLTISGLQPEDEADYYCSAYTSSDTVVFGGGTKLT |
| 42 | Anti CD122 light chain, | QSALTQPASVSGSPGQSIAISCTGTSSDIGHYDFVSWYQQHPGTAPKLIIYDINNRPSGIS |
| | clone P2C4_B6, | NRFSGSKSQNMASLTISGLQPEDEAQYYCSAYTSSDTVVFGGGTKLT |
| | P2C4_B6, P2C4_C12, | |
| | P2C4_D10, P2C4_E2, | |
| | P2C4_E3, P2C4_E8, | |
| | P2C4_G2, P2C4_G11, | |
| | P2C4_H1, P2C4_H2, | |
| | P2C4_H3 | |
| 43 | Anti CD122 light chain, | QSALTQPASVSGSPGQSIAISCTGTSSDIGHYDFISWYQQHPGTAPKLIIYDFNNRPSGIS |
| | clone P2C4_B12 | NRFSGSKSDNMASLTISGLQPEDEADYYCSAYTSSDTLVFGGGTKLT |
| 44 | Anti CD122 light chain, | QSALTQPASVSGSPGQSIAISCTGTSSDIGHYDFVSWYQCHPGTAPKLIIYQNNNRPSGI |
| | clone P2C4_C4 | SNRFSGSKSQNMASLTISGLQPEDEADYYCSAYTSSDTVVFGGGTKLT |
| 45 | Anti CD122 light chain, | QSALTQPASVSGSPGQSIAISCTGTSSDIGHYDFVSWYQGHPGTAPKLIIYDINNRPSGIS |
| | clone P2C4_C7 | NRFSGSKSDNMASLTISGLQPEDEADYYCSAYTSSDTVVFGGGTKLT |
| 46 | Anti CD122 light chain, | QSALTQPASVSGSPGQSIAISCTGTSSDIGDYDFVSWYQCHPGTAPKLIIYDINNRPSGIS |
| | clone P2C4_E6 | NRFSGSKSDNMASLIISGLQPEDEADYYCSAYTSSDTLVFGGGYKLT |
| 47 | Anti CD122 lightchain, | QSALTQPASVSGSPGQSIAISCTGTSSDIGHYDFVSWYQQHPGTAPKLIIYDINNRPSGIS |
| | clone P2C4_E7 | NRFSGSKSDDMASLTISGLQPEDEADYYCSAYTSSDTVVFGGQTKLT |
| 48 | Anti CD122 light chain, | QSALTQPASVSGSPGQSIAISCTGTSSDIGHYDFVSWYQQHPGTAPKLIIYDINNRASGIS |
| | clone P2C4_E9 | NRFSGSKSDNMASLTISGLQPEDEADYYCSAYTSSQTWFGGGTKLT |
| 49 | Anti CD122 light chain, | QSALTQPASVSGSPGQSIAISCTGTSSDIGHYDFVSWYQQHPGTAPKLIIYDINNRPSGIS |
| | clone P2C4_FB | NRFSGSKSDNMASLTISGLQPEDEADYYCSAYTSSDTVVFGGGTKLT |
| 50 | Anti CD122 light chain, | QSTLTQPASVSGSPGQSITISCTGTSSDIGHYDFVSWYQQHPGTAPKLIIYDINNRPSGIS |
| | clone P2C4_F11 | NRFSGSKSDNMASLTISGLQPEDEADYYCSAYTSSQTVVFGGGTKLT |
| 51 | Anti CD122 light chain, | QSALTQPASVSGSPGQSIAISCTGTSSDIGDYDFVSWYQGHPGTAPKLIIYDINNRPSGIS |
| | clone P2C4_C1D10 | NRFSGSKSDNMASLTISGLQPEDEADYYCSAYTSSDTVVFGGGTKLT |
| 52 | Anti CD122 light chain, | QSVLTQPPSVSGAPGQRVTISCTGTSSDIGHYDFVSWYQQLPGTAPKLLIYDINNRPSGV |
| | clone P2C4_FW2 | PDRFSGSKSGTSASLAITGLQAEDEADYYCSAYTSSDILVFGGGTKLT |
| 53 | Anti CD122 light chain, | DVVMTQSPLSLPVTPGEPASISCRSSQSLLHSNGYNYLDWYLQKPGQSPQILLIYLGSSR |
| | clone P1E7 | ASGVPQRFSGSGSGTDFTLKISRVEAEDVGVYYCMQALQTPRTFGQGTKLEIK |
| 54 | Anti CD122 light chain, | DIQMTQSPSSLSASVGDRVTITCQASQDISDYLNWYCQKPGKAPQILIYDASNLETGVPS |
| | clone P1B10 | RFSGSGSGTQFTFTISNLQPEDVATYYCQQYEDLPSFGGGTKVEIK |
| 55 | Anti CD122 light chain, | DIQLTQSPSSLSASVGDRVTITCRASQSISSYLNWYQQKPGKAPKALIYDASNLETGVPS |
| | clone P1F3 | RFSGSGSGTQFTLTIISLQPEDFATYFCLQDYIYPWTFGQGTKVEFK |
| 56 | Anti CD122 light chain, | QSVLTQPPSVSGAPGQRVTISCTGGSSNVGAGYDVHWYQQLPGTVPKLLIYDNTNRPSGY |
| | clone P1D10 | PDRFSASKSGTSASLVITGLQAEDEGDYYCQSYDSSLRASVFGGGTMLTVL |
| 57 | Anti CD122 light chain, | NFMLTQPHSVSESPGKTVTISCIGSSGSIASSSYVQWYQQRPGSAPTTVIYADNQRPSGV |
| | clone P1E1 | PQRFSGSVDS83NSASLTISGLKTEDEADYYCQSFQSSLYMIFGGGTKLTVL |
| 58 | Anti CD122 light chain, | QSVLTQPPSVSGAPGQRVTISCTGSRSNIGAGYDVHWYQHLPGTAPKLLIYDNSNRPSG |
| | clone P2B11 | VSDRESGSKSGTSASLAITGLQAEDEADYYCQSFQSSLRGVVFGGGIRLTVL |
| 59 | Anti CD122 light chain, | SYELTQPPSASGTPGQRVTISCSGSSSNIGSNTVNWYQQLPGTAPKLLIYSNNQRPSGV |
| | clone P2C9 | PQRFSGSKSGTBASLAISGLQSEDEADYYCAAWDDSLNGLWVFGGGTKLTVL |
| 60 | Anti CD122 light chain, | DVVMTQSPLSLPVTPGEPASISCRSSQRLLHSNGYNYVDWYLQKPGQSPQLLIYLGSNR |
| | clone P2C10 | ASGVPDRESGSGSGTDFTLKISRVEAEDVGVYYCMQGTHWPWTFGQGTKVEIK |
| 61 | Anti CD122 light chain, | DIQMTQSPPSLSASVGQRVTITCQASCQDINNYLNWYHQKPGKAPELLIYDASQLETGV |
| | clone P2C11 | PSRFSGSGSGTEFTFIISSLQPEDTGTYYCQQYDWLPLSYGGGTKVEIK |
| 62 | Anti CD122 light chain, | NFMLTQPHSVSGSPGKTITISCTRSSGNFASTYVQWYQQRPGSSPAIVIYDDDQRPSGV |
| | clone P2E6 | PDRFSGSIDRSSNSASLTISGLETEDEADYYCQSYDSSNFWVEGGGTKLTVL |
| 63 | Anti CD122 light chain, | EIVLTQSPSSLSASVGDRVTITCQASQDINNYLNWYQQKPGKAPKLLIYDASNLETGVPS |
| | clone P2E11 | KFSGSGSGTDFTFTISSLQPEDIATYYCQQYANLPSFGQGTKLEIK |
| 64 | Anti CD122 light chain, | EIVLTQSPLSLPVTPGEPASISCRSSQELLHSNGYNYLDWYLGKPGQSPQLLIYLGSNRA |
| | clone P2F9 | SGYPDRFSGSGSGTDFTLSISRVEAEDVGVYYCMQALQTPPTFGQGTKVEIK |
| 65 | Anti CD122 light chain, | DIQLTQSPSSLSASVGDRVTVICQASQDIGHNLNWYQQRPGKAPQLLIYDASNLETGVP |
| | clone P2P10 | SRFSGSGSGTQFTFTISSLQPEDIATYYCGQYDELPPDFGPGTKVEIK |
| 66 | Anti CD132 heavy chain, | QVQLQQWNGAGLLKPSETLSLTCAVYGGSFSGYYWSWIRGPPGKGLEWIGEINHSGSTN |
| | clone P1A3 | YNPSLKSRATISVDTSKNQFSLKLSSVTAADTAVYYCATSPGGYSGGYFQHWGQGTLVT |
| | | VSS |
| 67 | Anti CD132 heavy chain, | QVQLQESGPGLVKPSETLSLTCTVSGGSISSSSYYWGWIRQPPGKGLEWIGSIYYSGST |
| | clone P2B9 | YYNPSLKSRVTISVDTSKNQFSLKLSSVTAADTAVYYCAGDILTGYALDYWGQGTLVT |
| | | VSS |
| 68 | Anti CD132 heavy chain, | QVQLQQWGAGLLKPSETLSLTCAVYGGSFSGYYWSWIRQPPGKGLEWIGEINHFGSTN |
| | clones P1A3_B3, P1A3_B4, | YNPSLKSRATISVDTSKNQFSLKLSSVTAADTAVYYCATSPGGYSGGYFQHWGQGTL |
| | P1A3_E9 | VTVSS |
| 69 | Anti-CD132 heavy chain, | QVQLQQWGAGMLKPSETLSLTCAVYGGSFSGYYWSWIRQPPGKGLEWIGEINHFGST |
| | clone P1A3_E8 | NYNPSLKSRATISVDTSKNQFSLKLSSVTAADTAVYYCATSPGGYSGGYFQHWGQGTL |
| | | VTVSS |
| 70 | Anti CD132 heavy chain, | EVQLVESGGGLVQPGGSERLSCAASGGSFSGYYWSWVRQAPGKGLEWVSEINHSGST |
| | clone P1A3_FW2 | NYNPSLKSRFTISRDNSKNTLYLQMNSLRAEDTAVYYCARSPGGYSGGYFQHWGQGTL |
| | | VTVSS |
| 71 | Anti-CD132 heavy chain, | QVQLQQSGAEVKKPGSSVKVSCKASGGTESSYAISWVRQAPGQGLEWMGGFDPEDG |
| | clone P1A10 | ETIYAQKFQGRVTMTEDTSTDTAYMELSSLRSEDTAVYYCATDLRIPYYYDNPWGQGTL |
| | | VTVSS |
| 72 | Anti CD132 heavy chain, | QVQLVQSGGGVYQPGRSLRLSCAASGFTFSSYAMHWVRQAPGKGLEWVAVISYDGSN |
| | clone P1B6 | KYYADSVKGRFTISRDNSKNTLYLQMNSLRAEDTAVYYCARSLYYSHFDYWGQGTLVTV |
| | | SS |
| 73 | Anti CD132 heavy chain, | EVQLVETGPGLYKPSGTLSLTCAVSGGSISSSNWWSWVRQPPGKGLEWIGEIYHSGST |
| | clone P1C10 | NYNPSLKSRVTISVDKSKNQPFSLKLSSVTAADTAVYYCAREGPLSSSGPGAFDIWGQ |
| | | GTMVTVSS |
| 74 | Anti CD132 heavy chain, | QVQLQESGGGVVQPGRSLRLSCAASGFTFSNYGMHWVRQAPGKGLEWVAVISYDGTN |
| | clone P1D7 | KYYADSVKGRFTISRQNSKNTVYLQMNSLRAEDTAVYYCAKDGFDIWGQGTMVTVSS |
| 75 | Anti CD132 heavy chain, | EVQLVQSGGGVVQPGRSLRLSCAASGFTFSSYGMHWVRQAPGKGLEWVAVISYDQSN |
| | clone P1E6 | KYYADSVKGRFTISRDNSKNTLYLQMNSLRAEDTAVYYCARDVYGDYGAFDYWGQGTL |
| | | VTVSS |
| 76 | Anti CD132 heavy chain, | QLQLQESGGGVVQPGRSLRLSCAASGFTFSSYAMHWVRQAPGKGLEWVAVISYDGGN |
| | clone P2B2 | KYYADSVKGRFTISRDNSKNTLYLQMNSLRAEDTAVYYCAKSVAPPMDWVGKGTTVTV |
| | | SS |
| 77 | Anti CD132 heavy chain, | QVQLQQWGAGLLKPSETLSLTCAVYGESFSGYYWSWIRQPPGKGLEWIGEINHSGSTN |
| | clone P2B7 | YNPSLKSRVTISVDISKNQFSLKLSSVTAADTAVYYCARGPAGSSSSGYFDYWGQGTLV |
| | | TVSS |
| 78 | Anti CD132 heavy chain, | QVQLQESGPGLVKPSQTLSLTCTVSGGSISSGGYYWTWIRQHPGQGLEWIGFISWSGT |
| | clone P2D11 | TYYNPSLKNRVTISADTSKNHFSLNLTSVTAADTAVYYCARGSGRLVWGQGTLVTVSS |
| 79 | Anti CD132 heavy chain, | EVQLYQSGAEVKKPGABYKVSCKASGYTFTGYYMHWVRQAPGQGLEWMGIINPSGGS |
| | clone P2F10 | TSYAQKFQGRVTMTRDTSTSTVYMELSSLRSEDTAVYYCARADTAMGDAFDIWGQGTM |
| | | VTVSS |
| 80 | Anti CD132 heavy chain, | EVQLVQSGGGVVQPGRSLRLSCAASGFTFSSYAMHWVRQAPGKGLEWVAVISYDGSN |
| | clone P2H4 | KYYADSVKGRFTISRDNSKNTLYLQMNSLRAEDTAVYYCARSIGIGAFDIWGQGTMVTV |
| | | SS |
| 81 | Anti CD132 heavy chain, | QVQLQQWGAGLLKPSETESLTCTIYGGSFSGFYWSWIRQPPGKGLEWIGEINHSGSTNY |
| | clone P2D3 | NPSLKSRVTISVDTSKNQFSLKLSSVTAADTAIYYCARGPAGSTSSGYFDHWGGGTLVT |
| | | VSS |
| 82 | Anti CD132 heavy chain, | QVQLQQWGAGLLKPSETLSLTCAVYGGSLSGYYWSWIRQPPGKGLEWIGEINHSGSTN |
| | clone P1G4 | YNPSLKSRVTISVDTSKNQFSLKLSSVTAADTAVYYCARGSSSYYMDWWGKGTLVTVSS |
| 83 | Anti CD132 heavy chain, | QVQLQQWGAGLLKPSETLSLTCAVYGGSFSGYYWSWIRQPPGKGLEWIGEINHSGSTN |
| | clone P1B12 | YNPSLKSRVTISVILKLSSVTAADTAVYYCARGGSAYFQHWGQGTLVTVSS |
| 84 | Anti CD132 heavy chain, | QVQLQQWGAGLLKPSETLSLTCAVYGGSFSGYYWSWIRQPPGKGLEWIGEINHSGSTN |
| | clone P1C7 | YNPSLKSRVTISEDASKKQFSLTLTSVTAADTAVYYCARGPAGTGSSGYFDYWGQGTLV |
| | | TVSS |
| 85 | Anti CD132 light chiain, | DVVMTQSPLSLPVTPGEPASISCRSSQSLLHSNGYNYLDWYLQKPGQSPQLLIYLGSNR |
| | clones P1A3, P1A3_B3, | DSGVPDRFSGSGSGTDFTLKISRVEAEDVGYYYCMQGTHWPWTFGQGTKVEIK |
| | P1A3_E8, P1A3_E9 | |
| 86 | Anti CD132 light chain, | SYELTQPPSMSVSPGQTARITCSGDALPKQFAFWYQQKPGQAPVLVIYKDTERPSGIPE |
| | clone P2B9 | RFSGSSSGTTVTLTITGVQAEDEADYYCQSPDSSGTVEVFGGGTKLTVL |
| 87 | Anti CD132 light chain, | DVVMTQSPLSLPVTPGESVSISCRSSQSLLHSNGYNYLDWYLQKPGQSPQLLIYLGSNR |
| | clone P1A3_B4 | DSGVPDRFSGSGSGTDFTLKISRVEAEDVGVYYCMQGTHWPWTFGQGTKVEIK |
| 88 | Anti CD132 light chain, | DIQMTQSPSSLSASVGDRVTITCRSSQSLLHSNGYNYLDWYQQKPGKAPKLLIYLGSNR |
| | clone P1A3_FW2 | DSGVPSRFSGSGSGTDFTLTISSLQPEDFATYYCMQGTHWPWTFGQGTKVEIK |
| 89 | Anti CD132 light chain, | EIVLTQSPLSLPVTPGEPASISCRSSQSLLHSNGYNYLNWYLQKPGQSPQLLIYLGSDRA |
| | clone P1A10 | SGVPDRFSGSGSGTDFTLKISRVEAEDYGVYYCMQALQTPTTFGGGTKVEIK |
| 90 | Anti CD132 light chain, | EIVMTQSPLSLPVTPGEPASISCRSSQSLLHSNGYNYLDWYLQKPGQSPQLLMYLVSNR |
| | clone P1B6 | ASGVPERFSGSGSGTDFTLKSRVEAEDVGVYYCMQTLQTPLSFGQGTKLEIK |
| 91 | Anti CD132 light chain, | EIVLTQSPATLSLSPGERATLSCRASQSVSYHLAWYQQKPGQAPRLLIYDTSNRASGIPA |
| | clone P1C10 | RFSGSGSGTDFTLTINSLEPEDFAVYYCQQRYDWPLTFGGGTKVEIK |
| 92 | Anti CD132 light chain, | DIQMTQSPSFLSASVGDRVTITCRASQSISSWLAWYQQKPGKAPKLLIYDASRLEDGVPS |
| | clone P1D7 | RFSGTGFGTDFTFTLTTLQPDDIATYYCQQYDDLPYTFGQGTTVDIK |
| 93 | Anti CD132 light chain, | DVVMTQSPVSIPVTLGQPASISCKSSQSLLYFNGNTYLSWFQQRPGQSPRRLFYQVSN |
| | clone P1E8 | RDSGVPQRFSGSGSDTDFTLTISRVEAEDVGVYFCMQGTQWPPTFGQGTKVEIK |
| 94 | Anti CD132 light chain, | DVVMTQSPLSLPVTPGEPASISCRSSQSLLHSNGYNYLDWYLQKPGQSPHLLIYLGSNR |
| | clone P2B2 | ASGVPDRFSGSGSGTDFTLKISRVEAEDVGVYFCMQALRTPYTFGQGTKLEIK |
| 95 | Anti CD132 light cheln, | DVVMTQSPLSLPVTLGQPASISCRSSQSLVHSNGYNYLDWYLQKPGQSPQLLIYLGSNR |
| | clone P2B7 | ASGVPDRFSGSGSGTDFTLKISRVEAEDVGVYYCLQGSHWPWTFGQGTKVEIK |
| 96 | Anti CD132 light chain, | ETTLTQSPATLSVSPGERATLSCRASQSVSSNLAWYQQKPGQAPRLLIYGASSGATGIP |
| | clone P2C11 | DRPSGSGSGTDFTLTISRLEPEDFAVYYCQLYGSSLAFGGGTKVEIK |
| 97 | Anti CD132 light chain, | DIVMTHTPLSLPVTPGEPASISCRSSQTLFDSDDGKTYLDWYLQKPGQSPQLLMYTTSS |
| | clone P2P10 | RASGVPDRFSGSGSGTDFTLKISRVEAEDVGVYYCMQRLQFPLTFGQGTRLEFK |
| 98 | Anti CD132 light chain, | DVVMTQSPLSLPVTPGEPASISCRATQSLLRGNGHNYLDWYLQKPGQSPQLLIYLGSNR |
| | clone P2H4 | ASGVPQRFSGSGSGTDFTLKISRVEAEMQTLETPVTFGPGTKYDIK |
| 99 | Anti CD132 light chain, | DVVMTQSPLSLPVTPGEPASISQRSSQSLLHSNGYNYLDWYLQKPGQSPQLLIYLGSNR |
| | clone P2D3 | ASGVPDRFSGSGSGTDFTLKISRVEAEDVGVYYCMQGTHWPWTFGQGTKVEIK |
| 100 | Anti CD132 light chain, | DWMTQSPLSLPVTPGEPASISCRSSQSLLHSNGYNYLDWYLQKPGQSPQLLIYLGSNR |
| | clone P1G4 | ASGVPDRFSGSGSGTDFTLKISRVEAEDVGVYYCLQGTHWPWTFGQGTKVEIK |
| 101 | Anti CD132 light chain, | DVVMTQSPLSLPVTLGQPASISCRSSQSLLHSNGNNYLDWYLQKPGQSPQLLIYLGSNR |
| | clone P1B12 | ASGVPDRFSGSGSGTDFTLKISRVEAEDVGIYYCMQGTHWPWTFGQGTKVEIE |
| 102 | Anti CD132 light chain, | EIVLTQSPLSLPVTAGEPASISCRSSQSLLHSNGYNYLDWYLCKPGQSPQLLIYLASNRA |
| | clone P1C7 | SGVPDRFSGSGSGTDPTLKISRVEAEDVGVYYCMQGTHWPWTFGQGTKVEVK |
| 103 | HC CDR1 Anti | NYYMH |
| | CD122 clones | |
| | P2C4, P2C4_A4, | |
| | P2C4_B1, P2C4_B5, | |
| | P2C4_CA, P2C4_C7, | |
| | P2C4_D10, P2C4_E6, | |
| | P2C4_E7, P2C4_F8, | |
| | P2C4_A9, P2C4_B8, | |
| | P2C4_B12, P2C4-C1, | |
| | P2C4_C12, P2C4_E2, | |
| | P2C4_E8, P2C4_F11, | |
| | P2C4_G2, P2C4_G11, | |
| | P2C4_H1, P2C4_H2, | |
| | P2C4_H3, P2C4_C1D10, | |
| | P2C4_FW2 | |
| 104 | HC CDR1 Anti CD122 | TYAMH |
| | clone P2H7 | |
| 105 | HC CDR1 Anti CD122 | SYAMS |
| | clones P2D12, P1F3 | |
| 106 | HC CDR1 Anti CD122 | GYYWS |
| | clones P1G11, P1E1, | |
| | P2C10; | |
| | HC CDR1 Anti CD132 | |
| | clones P1A3, | |
| | P1A3_B3, P1A3_B4, | |
| | P1A3_E9, P1A3_E8, | |
| | P1A3_FW2, P2B7, | |
| | P1G4, P1B12, | |
| | P1C7 | |
| 107 | HC CDR1 Anti | NYYIH |
| | CD122 clones | |
| | P2C4_B8, P2C4_E9, | |
| | P2C4_E3 | |
| 108 | HC CDR1 Anti CD122 | SYAMH |
| | clone HC CDR1 | |
| | Anti CD132 | |
| | clones P1B6, | |
| | P2B2, P2H4 | |
| 109 | HC CDR1 Anti | SRSDHWG |
| | CD122 clones | |
| | P1B10, P2E 11 | |
| 110 | HC CDR1 Anti | SYYWS |
| | CD122 clone P1D10 | |
| 111 | HC CDR1 Anti | SYDLH |
| | CD122 clone P2B11 | |
| 112 | HC CDR1 Anti | SSNWWS |
| | CD122 clone P2C9; | |
| | HC CDR1 Anti | |
| | CD132 clone P1C10 | |
| 113 | HC CDR1 Anti | GNSATWN |
| | CD122 clone P2C11 | |
| 114 | HC CDR1 Anti | SGSWWS |
| | CD122 clone P2E6 | |
| 115 | HC CDR1 Anti | SYGIS |
| | CD122 clones | |
| | P2F9, P2F10 | |
| 116 | HC CDR2 Anti | AIMPSRGGTSYPQKFQG |
| | CD122 clones | |
| | P2C4, P2C4_A4, | |
| | P2C4_B1,P2C4_B5, | |
| | P2C4_CA, P2C4_C7, | |
| | P2C4_D10, P2C4_E6, | |
| | P2C4_E7, P2C4_F8, | |
| | P2C4_A9, | |
| | P2C4_B6, P2C4_B8, | |
| | P2C4_B9, P2C4_B12, | |
| | P2C4_C1, | |
| | P2C4_C12, | |
| | P2C4_E2, P2C4_E3, | |
| | P2C4_E8, P2C4_F11, | |
| | P2C4_G2, P2C4_G11, | |
| | P2C4_H1, P2C4_H2, | |
| | P2C4_H3, P2C4_C1D10, | |
| | P2C4_FW2 | |
| 117 | HC CDR2 Anti | WINTGNGNTKYSQNFQG |
| | CD122 clone P2H7 | |
| 118 | HC CDR2 Anti | AISGSGGSTYYADSVKG |
| | CD122 clone P2D12 | |
| 119 | HC CDR2 Anti | EINHSGSTNYNPSLKS |
| | CD122 clones P1G11, | |
| | P1D10, P1E1, P2C10; | |
| | HC CDR2 Anti CD132 | |
| | clones P1A3, | |
| | P1A3_FW2, P2B7, | |
| | P2D3, | |
| | P1G4, | |
| | P1B12, P1C7 | |
| 120 | HC CDR2 Anti | VISYDGSNKYYADSVKG |
| | CD122 clone | |
| | HC CDR2 Anti | |
| | CD132 clones P1B6, | |
| | P1E8, | |
| | F2H4 | |
| 121 | HC CDR2 Anti CD122 | SISYSGSTYYNPSLKS |
| | clones P1B10, | |
| | P2B11 | |
| 122 | HC CDR2 Anti | AISGSGGSTHYADSVKG |
| | CD122 clone P1F3 | |
| 123 | HC CDR2 Anti | LISYDGSNKYYADSVKG |
| | CD122 clone P2B11 | |
| 124 | HC CDR2 Anti CD122 | EIYHSGSTNYNPSLKS |
| | clone P2C9; | |
| | HC CDR2 Anti CD122 | |
| | clone P1C10 | |
| 125 | HC CDR2 Anti CD122 | RTYYRSKWNHDYAESVKS |
| | clone P2C11 | |
| 126 | HC CDR2 Anti CD122 | EIYHNGNTNYNPSLKS |
| | clone P2E6 | |
| 127 | HC CDR2 Anti CD122 | WISAYNGNTNYAQKLQG |
| | clones P2F9, | |
| | P2F10 | |
| 128 | HC CDR3 Anti CD122 | GEYYYDSSGYYY |
| | clones P2C4, | |
| | P2C4_A4, P2C4_B1, | |
| | P2C4_B5, P2C4_C4, | |
| | P2C4_C7; P2C4_D10, | |
| | P2C4_E6, | |
| | P2C4_E7, P2C4_P8, | |
| | P2C4_B6, | |
| | P2C4_E9, P2C4_B8, | |
| | P2C4_B12, P2C4_C1, | |
| | P2C4_C12, | |
| | P2C4_E2, | |
| | P2C4_E3, | |
| | P2C4_E8, P2C4_F11 | |
| | P2C4_G2, | |
| | P2C4_G11, | |
| | P2C4_H1, | |
| | P2C4_H2, | |
| | P2C4_H3, | |
| | P2C4_C1D10, | |
| | P2C4_FW2 | |
| 129 | HC CDR3 Anti | DLGQLERLYFW |
| | CD122 clone P2H7 | |
| 130 | HC CDR3 Anti | DLGDY |
| | CD122 clone P2D12 | |
| 131 | HC CDR3 Anti | SSSGQAFDI |
| | CD122 clone P1G11 | |
| 132 | HC CDR3 Anti | GEYYYDSSGYYN |
| | CD122 clone P2C4_A9 | |
| 133 | HC CDR3 Anti | DLGYSSSWYYYYYGMDV |
| | CD122 clone P1E7 | |
| 134 | HC CDR3 Anti CD122 | ESHPAAALVG |
| | clones P1B10, P2E11 | |
| 135 | HC CDR3 Anti CD122 | PAF |
| | clone P1F3 | |
| 136 | HC CDR3 Anti CD122 | GSNLDWFDDP |
| | clone P1C10 | |
| 137 | HC CDR3 Anti CD122 | ADRREGELRY |
| | clone P1E1 | |
| 138 | HC CDR3 Anti CD122 | EPITGTSDLFDY |
| | clone P2B11 | |
| 139 | HC CDR3 Anti CD122 | EGGLREEH |
| | clone P2C9 | |
| 140 | HC CDR3 Anti CD122 | GTDTAMADY |
| | clone P2C10 | |
| 141 | HC CDR3 Anti CD122 | DSKSAFDI |
| | clone P2C11 | |
| 142 | HC CDR3 Anti CD122 | VSGFDY |
| | clone P2E6 | |
| 149 | HC CDR3 Anti CD122 | APDYGDSSNYYYYYMDV |
| | clone P2F9 | |
| 144 | HC CDR3 Anti CD122 | DTSGDYSSGWYLGVPFDY |
| | clone P2F10 | |
| 145 | LC CDR1 Anti CD122 | TGTSSDIGHYDEVS |
| | clones P2C4, P2C4_A9, | |
| | P2C4_B1, P2C4_B6, | |
| | P2C4_B6, P2C4_B8, | |
| | P2C4_C12, P2C4_D10, | |
| | P2C4_E2, P2C4_E3, | |
| | P2C4_E8, P2C4_G2, | |
| | P2C4_G11, P2C4_H1, | |
| | P2C4_H2, P2C4_H3, | |
| | P2C4_C4, P2C4_CY, | |
| | P2C4_E7, P2C4_E8, | |
| | P2C4_F8, P2C4_F11 | |
| | P2C4_FW2 | |
| 146 | LC CDR1 Anti CD122 | RAGQAISSWLA |
| | clone P2H7 | |
| 147 | LC CDR1 Anti CD122 | QASQDIGNYLN |
| | clone P2D12 | |
| 148 | LC CDR1 Anti CD122 | TRSSGSIASNYVQ |
| | clone P1G11 | |
| 149 | LC CDR1 Anti CD122 | TGTSSDIGDYDFVS |
| | clones P2C4_A4, | |
| | P2C4_C1, P2C4_E8, | |
| | P2C4_C1D10 | |
| 150 | LC-CDR1 Anti CD122 | TGTSSDIGHYDFIS |
| | clone P2C4_B12 | |
| 151 | LC CDR1 Anti CD122 | RSSQSLLHSNGYNYLD |
| | clones PE7, P2F9; | |
| | LC CDR1 Anti CD132 | |
| | clones P1A3, P1A3_B3, | |
| | P1A3_E8, P1A3_E9, | |
| | P1A3_B4, P1A3_FW2, | |
| | P1B6, P2B2, | |
| | P2D3, P1G4, P1C7 | |
| 152 | LC CDR1 Anti CD122 | QASQQISDYLN |
| | clone P1B10 | |
| 153 | LC CDR1 Anti CD122 | RASQSISSYLN |
| | clone PAF3 | |
| 154 | LC CDR1 Anti CD122 | TGGSSNVGAGYDVH |
| | clone P1D10 | |
| 155 | LC CDR1 Anti CD122 | TGSSGSIASSYVQ |
| | clone P1E1 | |
| 158 | LC CDR1 Anti CD122 | TGSRSNIGAGYDVH |
| | clone P2B11 | |
| 157 | LC CDR1 Anti CD122 | SGSSSNIGSNTVN |
| | clone P2C9 | |
| 158 | LC CDR1 Anti CD122 | RSSQRLLHSNGYNYVD |
| | clone P2C10 | |
| 159 | LG CDR1 Anti CD122 | QASQDINNYLN |
| | clones P2C11, P2E11 | |
| 160 | LC CDR1 Anti CD122 | TRSSGNFASTYVQ |
| | clone | |
| | P2E6 | |
| 161 | LG CDR1 And CD122 | QASQDIGHNEN |
| | clone P2F10 | |
| 162 | LC CDR2 Anti CD122 | DINNRPS |
| | clones P2C4, P2C4_A9, | |
| | P2C4_A4, P2C4_C1, | |
| | P2C4_B5, P2C4_B6, P2C4_B8, | |
| | P2C4_C12, P2C4_D10, | |
| | P2C4_E2, P2C4_E3, | |
| | P2C4_E8, P2C4_G2, | |
| | P2C4_G11, P2C4_H1, | |
| | P2C4_H2, P2C4_H3 | |
| | P2C4_C7, P2C4_E6, | |
| | P2C4_E7, P2C4_E8, | |
| | P2C4_F11, P2C4_C1D10; | |
| | P2C4_FW2 | |
| 163 | LC CDR2 Anti CD122 | KASNLES |
| | clone P2H7 | |
| 164 | LC CDR2 Anti CD122 | DASNLET |
| | clones P2C12, P1B10, | |
| | P1F3, P2E11, P2F10 | |
| 165 | LC CDR2 Anti CD122 | DQNQRPT |
| | clone P1G11 | |
| 166 | LC CDR2 Anti CD122 | DNNNRPS |
| | clones P2C4_B1, | |
| | P2C4_C4 | |
| 167 | LC CDR2 Anti CD122 | DENNRPS |
| | clone P2C4_B12 | |
| 168 | LC CDR2 Anti CD122 | DINNRAS |
| | clone P2C4_E9 | |
| 169 | LC CDR2 Anti CD122 | LGSSRAS |
| | clone P1E7 | |
| 170 | LC CDR2 Anti CD122 | QNTNRPS |
| | clone P1D10 | |
| 171 | LC CDR2 Anti CD122 | AQNQRPS |
| | clone P1E1 | |
| 172 | LC CDR2 Anti CD122 | DNSNRPS |
| | clone P2B11 | |
| 173 | LC CDR2 Anti CD122 | SNNQRPS |
| | clone P2C9 | |
| 174 | LC CDR2 Anti CD122 | LGSNRAS |
| | clones P2C10, P2F8; | |
| | LC CDR2 Anti CD132 | |
| | clones P2B2, P2B7, | |
| | P2H4; P2C3, P1G4, | |
| | P1B12 | |
| 175 | LC CDR2 Anti CD122 | DASQLET |
| | clone P2C11 | |
| 176 | LC CDR2 Anti CD122 | DQDQRPS |
| | clone P2E6 | |
| 177 | LC CDR3 Anti CD122 | SAYTSSDTEV |
| | clones P2C4, P2C4_A9, | |
| | P2C4_A4, P2C4_C1, | |
| | P2C4_B1, P2C4_B12, | |
| | P2C4_E6, P2C4_FW2 | |
| 178 | LC CDR3 Anti CD122 | QQYQSYPYT |
| | clone P2H7 | |
| 179 | LC CDR3 Anti CD122 | LQLYDYPLT |
| | clone P2D12 | |
| 180 | LC CDR3 Anti CD122 | QSSHSTAVV |
| | clone P1G11 | |
| 181 | LC CDR3 Anti CD122 | SAYTSSDTVV |
| | clones P2C4_B5, | |
| | P2C4_B6, P2C4_B8, | |
| | P2C4_C12, P2C4_D10, | |
| | P2C4_E2, P2C4_E3, | |
| | P2C4_E8, P2C4_G2, | |
| | P2C4_G11; P2C4_H1, | |
| | P2C4_H2, P2C4_H3, | |
| | P2C4_C4, | |
| | P2C4_C7, P2C4_ET, | |
| | P2C4_E9, P2C4_F8, | |
| | P2C4_F11, P2C4_C1010 | |
| 182 | LC CDR3 Anti CD122 | MQALQTPRT |
| | clone P1E7 | |
| 183 | LC-CDR3 Ant-CD122 | QQYEDLPS |
| | clone P1B10 | |
| 184 | LC CDR3 Anti CD122 | LQDYIYPWT |
| | clone P1F3 | |
| 185 | LC CDR3 Anti CD122 | QSYDSSLRASV |
| | clone P1D10 | |
| 186 | LC CDR3 Anti CD122 | QSFDSSLYMI |
| | clone P1E1 | |
| 187 | LC CDR3 Anti CD122 | QSFDSSLRGVV |
| | clone P2B11 | |
| 188 | LC CDR3 Anti CD122 | AAWDDSINGLWV |
| | clone P2C9 | |
| 189 | LC CDR3 Anti CD122 | MQGTHWPWT |
| | clone P2C10; LC CDR3 | |
| | Anti CD132 clones P1A3, | |
| | P1A3_B3, P1A3_E8, P1A3_E9, | |
| | P1A3_B4, P1A3_FW2, | |
| | P2D3, P1B12, P1C7 | |
| 190 | LC CDR3 Anti CD122 | QQYDWLPLS |
| | clone P2C11 | |
| 191 | LC CDR3 Anti CD122 | QSYDSSNFWV |
| | clone P2B6 | |
| 192 | LC CDR3 Ani CD122 | QQYANLPS |
| | clone P2611 | |
| 193 | LC CDR3 Anti CD122 | MQALQTPPT |
| | clone P2F9 | |
| | LC CDR3 Anti | QQYDFLPPD |
| | CD122 clone P2F10 | |
| 195 | MC CDR1 Anti | SSSYYWG |
| | CD132 clone P2B9 | |
| 196 | HG CDR1 Anti | SYAIS |
| | CD132 clone P1A10 | |
| 197 | HC CDR1 Anti | NYGMH |
| | CD132 clone | |
| 198 | RC CDR1 Anti | SYGMH |
| | CD132 clone P168 | |
| 199 | HC CDR1 Anti | SGGYYWT |
| | CD132 clone P2C11 | |
| 200 | HC CDR1 Anti | GYYMH |
| | CD132 clone P2P10 | |
| 201 | HC CDR1 Anti | GFYWS |
| | CD132 clone P2D3 | |
| 202 | HC CDR2 Anti | SIYYSGSTYYNPSLK |
| | CD 132 clone P2B9 | |
| 203 | HC CDR2 Anti | EINHFGSTNYNPSLKS |
| | CD132 clones P1A3_B3, | |
| | P1A3_B4, P1A3_69, | |
| | P1A3_B8 | |
| 204 | HC CDR2 Anti | GFDPEDGETIVAQKFQG |
| | CD132 clone P1A10 | |
| 206 | HC CDR2 Anti | VISYDGINKYYADSVKG |
| | CD132 clone P107 | |
| 207 | HC CDR2 Anti | VISYDGGNKYYADSVKG |
| | CD132 clone P2B2 | |
| 208 | HC CDR2 Anti | FISWSGTTYYNPSLKN |
| | CD132 clone P2C11 | |
| 209 | HC CDR2 Anti | IINPSGGSTSYAQKFQG |
| | CD132 clone P2F10 | |
| 210 | HC CDR3 Anti | SPGGYSGGYFQH |
| | CD132 clones P1A3, | |
| | P1A3_B3, P1A3_B4, | |
| | P1A3_E9, P1A3_E8, | |
| | P1A3_FW2 | |
| 211 | HC CDR3 Anti | DILTGYALDY |
| | CD132 clone P2B9 | |
| 212 | HC CDR3 Anti | DLRIPYYYDNP |
| | CD132 clone P1A10 | |
| 213 | HC CDR3 Anti | SLYYSHFDY |
| | CD132 clone P1B6 | |
| 214 | HC-CDR3 Anti | EGPLSSSGPGAPDI |
| | CD132 clone P1C10 | |
| 215 | HC CDR3 Anti | DGFDI |
| | CD132 clone P1D7 | |
| 216 | HC CDR3 Anti | DVYGDYGAFDY |
| | CD132 clone P1B8 | |
| 217 | HC CDR3 Anti | SVAPPMDV |
| | CD132 clone P2B2 | |
| 218 | HC CDR3 Anti | GPAGSSSSGYFDY |
| | CD132 clone P2B7 | |
| 219 | HC CDR3 Anti | GSGRLV |
| | CD132 dlone P2D11 | |
| 220 | HC CDR3 Anti | ADTAMGDAFDI |
| | CD132 clone P2F10 | |
| 221 | HC CDR3 Anti | SIGIGAFDI |
| | CD132 clone P2H4 | |
| 222 | HC CDR3 Anti | GPAGSTSSGYFDH |
| | CD132 clone P2D3 | |
| 223 | HC CDR3 Anti | GSSSYYMDV |
| | CD132 clone PIG4 | |
| 224 | HC CDR3 Anti | GGSAYFQH |
| | CD132 clone P1B12 | |
| 225 | HC CDR3 Anti | GPAGTGSSGYFDY |
| | CD132 clone P107 | |
| 226 | LC CDR1 Anti | SGDALPKQFAF |
| | CD132 clone P2B9 | |
| 227 | LC CDR1 Anti | RSSQSLLHSNGYNYLN |
| | CD132 clone P1A10 | |
| 228 | LC-CDR1 Anti | RASQSVSYHLA |
| | CD132 clone P1C10 | |
| 229 | LC CDR1 Anti | RASQSISSWLA |
| | CD132 clone P1D7 | |
| 230 | LC CDR1 Anti | KSSQSLLYFNGNTYLS |
| | CD132 clone P1B8 | |
| 231 | LC CDR1 Anti | RSSQSLVHSNGYNYLD |
| | CD132 clone P2B7 | |
| 232 | LC CDR1 Anti | RASQSVSSNLA |
| | CD132 clone P2D14 | |
| 233 | LC CDR1 Anti | RSSQTLFDSDDGKTYLD |
| | CD132 clone P2F10 | |
| 234 | LC CDR1 Anti | RATQSLLHGNGHNYLD |
| | CD132 clone P2H4 | |
| 235 | LC CDR1 Anti | RSSQSLLHSNGNNYLD |
| | CD132 clone P1B12 | |
| 236 | LC CDR2 Anti | LGSNRDS |
| | CD132 clones P1A3, | |
| | P1A3_B3, P1A3_E8, | |
| | P1A3_E9, P1A3_B4, | |
| | P1A3_FW2 | |
| 237 | LC CDR2 Anti | KDTERPS |
| | CD132 clone P2B9 | |
| 238 | LC CDR2 Anti | LGSDRAS |
| | CD132 clone P1A10 | |
| 239 | LC CDR2 Anti | LVSNRAS |
| | CD13 clone P1B6 | |
| 240 | LC CDR2 Anti | DTSNRAS |
| | CD132 clone P1C10 | |
| 241 | LC CDR2 Anti | DASRLED |
| | CD13 P107 | |
| 242 | LC CDR2 Anti | QVSNRDS |
| | CD132 clone P1B8 | |
| 243 | LC CDR2 Anti | GASSGAT |
| | CD132 clone P2D11 | |
| 244 | LC CDR2 Anti | TTSSRAS |
| | CD132 clone P2P10 | |
| 245 | LC CDR Anti | LASNRAS |
| | CD132 clone P1C7 | |
| 247 | LC CDR3 Anti | QSPDSSGTVEV |
| | CD132 clone P2B9 | |
| 248 | LC CDR3 Anti | MQALQTPTT |
| | CD132 clone P1A10 | |
| 249 | LC CDR3 Anti | MQTLQTPLS |
| | CD132 clone P1B6 | |
| 250 | LC CDR3 Anti | QQRYDWPLI |
| | CD132 clone P1C10 | |
| 251 | LC CDR3 Anti | QQYDDLPYT |
| | CD132 clone P1D7 | |
| 252 | LC CDRS Anti | MQGTQWPPT |
| | CD132 clone P1E8 | |
| 253 | LC CDR3 Anti | MQALRTPYT |
| | CD132 clone P2B2 | |
| 254 | LC CDR3 Anti | LQGSHWPWT |
| | CD132 clone P2B7 | |
| 255 | LC CDR3 Anti | QLYGSSLA |
| | CD132 clone P2D11 | |
| 256 | LC CDR3 Anti | MQRLQFPLT |
| | CD132 clone P2F10 | |
| 257 | LC CDR3 Anti | MQTLETPVT |
| | CD132 clone P2H4 | |
| 258 | LC CDR3 Anti | LQGTHWPWI |
| | CD132 clone P1G4 | |
| 259 | CH2 domain P2C4 | PCPAPEAAGGPSVFLFPPKPKDTLMISRTPEVTCVVVDVSHEDPEVKFNWYVDGVEVH |
| | | NAKTKPREEQYNSTYRVVSVLTVLHQDWLNGKEYKCKVSNKALPAPIEKTISKAK |
| 260 | CH3 domain P2C4 | GQPREPQVYTLPPCRDELTKNQVSLWCLVKGFYPSDIAVWESNGQPENNYKTTPPVL |
| | | DSDGSFFLYSKLTVDKSRWQQGNVFSCSVMHEALHNHYTQKSLSLSPGK |
| 261 | CH2 domain P1A3 | PCPAPEAAGGPSVFLFPPKPKQTLMISRTPEVTCVVVDVSHEDPEVKENWYVDGVEVH |
| | | NAKTKPREEQYNSTYRVVSVLTVLHQDWLNGKEYKCKVSNKALPAPIEKTISKAK |
| 262 | CH3 domain P1A3 | GQPREPQVCTLPPSRDELTKNQVSLSCAVKGFYPSDIAVEWESNGQPENNYKTTPPVL |
| | | DSDGSFFLVSKLTVDKSRWQQGNVFSCSVMHEALHNHYTQKSLSLSPGK |
| 263 | CH2 domain P1A10 | PCPAPEAAGGPSVFLFPPKPKQTLMISRTPEVTCVVVDVSHEDPEVKENWYGVEVH |
| | | NAKTKPREEQYNSTYRVVSVLTVLHQDWLNGKEYKCKVSNKALPAPIEKTISKAK |
| 244 | CH3 domain P1A10 | GQPREPQVCTLPPSRDELTKNQVSLSCAVKGFYPSDIAVEWESNGQPENNYKTTPPVL |
| | | DSDGSPFLVSKLIVDKSRWQQGNVFSCSVMHEALENHYTQKSLSLSPGK |
| 265 | Anti | EVQLVQSGAEVKKPGSSVKVSCKASGYTFTNYYMHWVRGAPGQGLEWMGAIMPSRG |
| | CD122 clone P2C4 | GTSYPQKFQGRVTMTGDISTSTVYMELSSLRSEDTAVYYCARGEYYYDSSGYYYWGQ |
| | | GTLVTVSSGGGGSGGGGSGGGGSQSALTQPASVSGSPGQSIAISCTGTSSDIGHYDFV |
| | | SWYQCHPGTAPKLIIYDINNRPSGISNRFSGSKSQNMASLTISGLQPEDEADYYCSAYTS |
| | | SDTLVFGGGTKLTVLNSGAGTAAATHTCPPCPAPEAAGGPSVFLFPPKPKDILMISRTP |
| | | EVTCVVVDVSHEDPEVKFNWYVDGVEVHNAKTKPREEGYNSTYRVVSVLTVLHQDWLN |
| | | GKEYKCKVSNKALPAPIEKTISKAKGQPREPQVYTLPPCRDELTKNQVSLWCLVKGFYP |
| | | SDIAVEWESNGQPENNYKTTPPVLDSDGSFFLYSKLTVDKSRWQQGNVFSCSVMHEAL |
| | | HNHYTQKSLSLSPGK |
| 266 | Anti CD122 | EVQLVQSGTEVKKPGASVKVSCKASGYTFTTYAMHWVRQAPGQSLEWMGW |
| | clone P2H7 | INTGNGNTKYSQNFQGRVTMTRDTSISTAYMELSRLRSDDTAVYYCARDL |
| | | GQLERLYFWGQGTLVTVSSGGGGSGGGGSGGGGSDIQMTQSPSTLSASVG |
| | | DRVTLSCRAGQAISSWLAWYQQKPGKAPKLLIYKASNLESGVPSRFSGGG |
| | | SGAEFTLTISSLQPDDFATYYCQQYQSYPYYFGQGTKLEIR |
| 267 | Anti CD122 | HVQLVETGGGLVQPGGSLRLSCAASGFTFSSYAMSWVRQAPGKGLEWVSA |
| | clone P2D12 | ISGSGGSTYYADSVKGRATISRDNSKNTLYLQMNSLRAEDTAVYYCARDL |
| | | GDYWGQGTLVTVSSGGGGSGGGGSGGGGSDIQLTQSPSSLSASVGDRYTI |
| | | TCQASQDIGNYLNWYQLKPGKAPKLLIYDASNLETGVPSRFSGSGSGTDF |
| | | TFTISSLQPEDIATYYCLQLYDYPLTFGGGTKVEIK |
| 268 | Anti CD122 | QVQLQQWGAGLLKPSETLSLTCAVYGGSFSGYYWSWIRQPPGKGLEWIGE |
| | clone P1G11 | INHSGSTNYNPSLKSRVTISVDTSKNQFSLKLSSVTAADTAVYYCARSSS |
| | | GDAFDIWGQGTMVTVSSGGGGSGGGGGGGGSNFMLTQPHSVSESPGKTVT |
| | | ISCTRSSGSIASNYVQWYQQRPGSSPTTVIFDDNQRPTGVPDRFSAAIDT |
| | | SSSSASLTISGLTAEDEADYYCQSSHSTAVVFGGGTKLTVL |
| 269 | Anti CD122 clone | EVQLVQSGAEVKKPGSSVKVSCKASGYTFTNYYMHWVRQAPGQGLEWMGA |
| | P2C4_A4 | IMPSRGGTSYPQKFQGRVTMTGDTSTSTVYMELSSLRSEDTAVYYCARGE |
| | | YYYDSSGYYYWGQGTLVTVSSGGGGSGGGGSGGGGSQSALTQPASVSGSP |
| | | GQSIAISCTGTSSDIGDYDFVSWYQQHPGTAPKLIIYDINNRFSGISNRF |
| | | SGSKSQNMASLTISGLQPEDEADYYCSAYTSSDTLVFGGGTKLTVL |
| 270 | Anti CD122 clone | EVQLVQSGAEVKKPGSSVKVSCKASGYTFTNYYMHWVRQAPGQGLEWMGA |
| | P2C4_A9 | IMPSRGGTSYPQKFQGRVTMTGDTSTSTVYMELSSLRSEDTAVYYCARGE |
| | | YYYDSSGYYNWGQGTLVTVSSGGGGSGGGGSGGGGSQSALTQPASVSGSP |
| | | GQSIAISCTGTSSDIGHYDFVSWYQQHPGTAPKLIIYDINNRPSGISNRF |
| | | SGSKSQNMASLTISGLQPEDEADYYCSAYTSSDTVVFGGGTKLTVL |
| 271 | Anti CD122 | EVQLVQSGAEVKKPGSSYKVSCKASGYTFTNYYMHWVRQAPGQGLEWMGA |
| | clone P2C4 B1 | IMPSRGGTSYPQKFQGRVTMTGDTSTSTVYMELSSLRSEDTAVYYCARGE |
| | | YYYDSSGYYYWGQGTLVYVSSGGGGSGGGGSGGGGSQSALTQPASVSGSP |
| | | GQSIAISCTGTSSDIGHYDFVSWYQQHPGTAPKLIIYDNNNRPSGISNRF |
| | | SGSKSQNMASLTISGLQPEDEADYYCSAYTSSDTLVFGGGTKLTVL |
| 272 | Anti CD122 | EVQLVQSGAEVKKPGSSVKVSCKASGYTFTNYYMHWVRQAPGQGLEWMGA |
| | clone P2C4_B5 | IMPSRGGTSYPQKFQGRVIMIGQTSYSTVYMELSSLRSEDIAVYYCARGE |
| | | YYYDSSGYYYWGQGILVTVSSGGGGSGGGGSGGGGSQSALTQPASVSGSP |
| | | GQSITISCTGTSSDIGHYDFVSWYQQHPGTAPKLIIYDINNRPSGISNRF |
| | | SGSKSDNMASLTISGLQPEDEADYYCSAYTSSDTVVFGGGTKLTVL |
| 273 | Ant CD122 | EVQLVQSGAEVKKPGSSYKVSCKASGYTFTNYYIHWVRQAPGQGLEWMGA |
| | clone P2C4_B6 | IMPSRGGTSYPQKFQGRVTMTGDTSTSTVYMELSSLRSEDTAVYYCARGE |
| | | YYYDSSGYYYWGQGTLVTVSSGGGGSGGGGSGGGGSQSALTQPASVSGSP |
| | | GQSIAISCTGTSSDIGHYDFVSWYQQHPGTAPKLIIYDINNRPSGISNRF |
| | | SGSKSQNMASLTISGLQPEDEADYYCSAYTSSDTVVFGGGTKLTVL |
| 274 | Anti CD122 | EVQLVQSGAEVKKPGSSVKVSCKASGYTFTNYYMHWVRQPPGQGLEWMGA |
| | clone P2C4_B8 | IMPSRGGTSYPQKFQGRVTMTGDTSTSTVYMELSSLRSEDTAVYYCARGE |
| | | YYYDSSGYYYWGQGTLVTVSSGGGGSGGGGSGGGGSQSALTQPASVSGSP |
| | | GQSIAISCTGTSSDIGHYQFVSWYQQHPGTAPKLIIYDINNRPSGISNRF |
| | | SGSKSDNMASLTISGLQPEDEADYYCSAYTSSDTVVFGGGTKLTVL |
| 275 | Anti CD122 | EVQLVQSGAEVKKPGSTYKVSCKASGYTFTNYYMHWVRGAPGQGLEWMGA |
| | clone P2C4_B12 | IMPSRGGTSYPQKFQGRVTMTGDTSTSTVYMELSSLRSEDTAVYYCARGE |
| | | YYYDSSGYYYWGQGTLVTVSSGGGGSGGGGSGGGGSQSALTQPASVSGSP |
| | | GQSIAISCTGTSSDIGHYDFISWYQQHPGTAPKLIIYDENNRPSGISNRE |
| | | SGSKSDNMASLTISGLQPEDEADYYCSAYTSSDTLVFGGGTKLYVL |
| 276 | Ant CD122 | EVQLVQSGAEVKKPGSSVKVSCKASGYTFTNYYMHWVRGAPGQGLEWMGA |
| | clone P2C4_C1 | IMPSRGGTSYPQKFQGRVTMTGDTSTSTVYMELSSLRSEDTAVYYCARGE |
| | | YYYDSSGYYYWGQGYPVIVSSGGGGSGGGGSGGGGSQSALTQPASVSGSP |
| | | GQSIAISCTGTSSDIGDYDFVSWYQQHPGTAPKLIIYDINNRPSGISNRF |
| | | SGSKSDNMASLTISGLQPEDEADYYCSAYTSSDTLVFGGGTKLTVL |
| 277 | Anti CD122- | EVQLVQSGAEVKKPGSSVKVSCKASGYTFTNYYMHWVRQAPGQGLEWMGA |
| | clone P2C4_C4 | IMPSRGGTSYPQKFQGRVTMTGDTSTSTVYMELSSLRSEDTAVYYCARGE |
| | | YYYDSSGYYYWGQGTLVYVSSGGGGSGGGGSGGGGSQSALTQPASVSGSP |
| | | GQSIAISCIGTSSDIGHYDFVSWYQQHPGTAPKLIIYDNNNRPSGISNRF |
| | | SGSKSQNMASLTISGLQPEDEADYYCSAYTSSDTVVFGGGTKLTVL |
| 278 | Anti CD122 | EVQLVQSGAEVKKPGSSVKVSCKASGYTFTNYYMHWVRQAPGQGLEWMGA |
| | clone P2C4_C7 | IMPSRGGTSYPQKFQGRVTMTGQTSTSTVYMELSSLRSEDTAVYYCARGE |
| | | YYYDSSGYYYWGQGTLVTVSSGGGGSGGGGSGGGGSQSALTQPASVSGSP |
| | | GQSIVISCTGTSSDIGHYDFVSWYQQHPGTAPKLLIYDINNRPSGISNRF |
| | | SGSKSQNMASLTISGLQPEDEADYYCSAYTSSDTVVFGGGTKLTVLAAAH |
| | | HHH |
| 279 | Anti CD122 | EVQLVQSGAEVKKPGSSYKVSCKASGYTFTNYYMHWVRQAPGQGLEWMGA |
| | clone P2C4_C12 | IMPSRGGTSYPQKFQGRVTMTGDTSTSTVYMELSNLRSEDTAVYYCARGE |
| | | YYYDSSGYVYWGQGTLVTVSNGGGGSGGGGSGGGGSQSALTQPASVSGSP |
| | | GQSIAISCTGTSSDIGHYDFVSWYQQHPGTAPKLIIYDINNRPSGISNRF |
| | | SGSKSQNMASLTISGLQPEDEADYYCSAYTSSDTVVFGGGTKLTYL |
| 280 | Anti CD122 | EVQLVQSGAEVKKPGSSVKVSCKASGYTFTNYYMHWVRQAPGQGLEWMGA |
| | clone P2C4_D10 | IMPSRGGTSYPQKFQGRVTMTGDYSTSIVYMELSSLRSEDTAVYYCARGE |
| | | YYYDSSGYYYWGQGTLVTVSSGGGGSGGGGSGGGGSQSALTQPASVSGSP |
| | | GQSIAISCIGTSSDIGHYDFVSWYQQHPGTAPKLIIYDINNRPSGISNRF |
| | | SGSKSDNMASLTISGLQPEDEADYYCSAYTSSDTVVFGGGTKLTVI |
| 281 | Anti CD122 | EVQLVQSGAEVKEPGSSVKVSCKASGYTFTNYYMHWVRQAPGQGLEWMGA |
| | clone P2C4_E2 | IMPSRGGTSYPQKFQGRVIMIGDISTSTVYMELSSLRSEDTAVYYCARGE |
| | | YYYDSSGYYYWGQGTLVTVSSGGGGSGGGGSGGGGSQSALTQPASVSGSP |
| | | GQSIAISCTGTSSDIGHYDFVSWYQQHPGTAPKLIIYDINNRPSGISNRF |
| | | SGSKSDNMASLTISGLQPEDEADYYCSAYTSSDTVVFGGGTKLTVL |
| 282 | Anti CD122 | EVQLVQSGAEVKKPGSSVKVSCKASGYTFTNYYIHWVRQAPGQGLEWMGA |
| | clone P2C4_E3 | IMPSRGGTSYPQKFQGRVTMTGDYSTSTVYMELNSLRSEDTAVYYCARGE |
| | | YYYDSSGYYYWGQGTLVTVSSGGGGSGGGGSGGGGSQSALTQPASVSGSP |
| | | GQSIAISCTGTSSDIGHYDFVSWYQQHPGTAPKLIIYDINNRPSGISNRF |
| | | SGSKSQNMASLTISGLQPEDEADYYCSAYTSSDTVVFGGGTKLTVL |
| 283 | Anti CD122 | EVQLVQSGAEVKKPGSSVKYSCKASGYTFTNYYMHWVRQAFGQGLEWMGA |
| | clone P2C4_E6 | IMPSRGQTSYPQKFQGRVTMTGDISTSIVYMELSSLRSEDTAVYYCARGE |
| | | YYYDSSGYYYWGQGTLVTVSSGGGGSGGGGSGGGGSQSALTQPASVSGSP |
| | | GQSIAISCTGTSSDIGDYDFVSWYQQHPGTAPKLIIYDINNRPSGISNRF |
| | | SGSKSDNMASLIISGLQPEDEADYYCSAYTSSDTLVEGGGTKLTVL |
| 284 | Anti CD122 | EVQLVQSGAEVKKPGSSYKVSCKASGYTFTNYYMHWVRQAPGQGLEWMGA |
| | clone P2C4_E7 | IMPSRGGTSYPQKFQGRVIMIGDTSTSTVYMELSSLRSEDIAVYYCARGE |
| | | YYYDSSGYYYWGQGTLVTVSSGGGGSGGGGSGGGGSQSALTQPASVSGSP |
| | | GQSIAISCTGTSSDIGHYDFVSWYQQHPGTAPKLIIYDINNRPSGISNRF |
| | | SGSKSDDMASLTISGLQPEDEADYYCSAYTSSDTVVFGGGTKLTVL |
| 285 | Anti CD122 | EVQLVQSGAEVKKPGSSVKVSCKASGYTFTNYYMHWVRQAFGQGLEWMGA |
| | clone P2C4_E8 | IMPSRGGTSYPQKFQGRVTMTGDTSTSIVYMELSSLRSEDTAVYYCARGE |
| | | YYYQSSGYYYWGPGTLVTVSSGGGGSGGGGSGGGGSQSALTQPASVSGSP |
| | | GQSIAISCTGTSSDIGHYDFVSWYQQHPGTAPKLIIYDINNRPSGISNRF |
| | | SGSKSQNMASLTISGLQPEDEADYYCSAYTSSDTVVFGGGTKLTVL |
| 286 | Anti CD122 | EVQLVQSGAEVKKPGSSVKVSCKASGYTFTNYYIHWVRQAPGQGLEWMGA |
| | clone P2C4_E9 | IMPSRGGTSYPQKFQGRVTMTGDTSTSTVYMELSSLRSEDTAVYYCARGE |
| | | YYYDSSGYYYWGQGTLVTVSSGGGGSGGGGSGGGGSQSALTQPASVSGSP |
| | | GQSIAISCTGTSSDIGHYQFVSWYQQHPGTAPKLIIYDINNRASGISNRF |
| | | SGSKSDNMASLTISGLQPEDEADYYCSAYTSSDTVVFGGGTKLTVI |
| 287 | Ant CD122 | EVQLVQSGAEVKKPGSSVKVSCKASGYTFTNYYMHWVRQAPGQGLEWMGA |
| | clone P2C4_F8 | IMPSRGGTSYPQKFQGRVTMTGQTSTSTVYMELSSLRSEDTAVYYCARGE |
| | | YYYDSSGYYYWGQGTLVTVSSGGGGSGGGGSGGGGSQSALTQPASVSGNP |
| | | GQSIAISCTGTSSDIGHYDFVSWYQQHPGTAPKLIIYDINNRPSGISNRF |
| | | SGSKSQNMASLTISGLQPEDEADYYCSAYTSSQTVYFGGGTKLTVL |
| 288 | Anti CD122 | EVQLVQSGAEVKKPGSSVKVSCKASGYTFTNYYMHWVRQAPGQGLEWMGA |
| | clone P2C4_F11 | IMPSRGGTSYPQKFQGRVIMIGDISTSTVYMELSSLRSEDTAMYYCARGE |
| | | YYYDSSGYYYWGQGTLVTVSSGGGGSGGGGSGGGGSQSTLTQPASVSGSP |
| | | GQSITISCTGTSSDIGHYDFVSWYQQHPGTAPKLIIYDINNRPSGISNRF |
| | | SGSKSDNMASLTISGLQPEDEADYYCSAYTSSDTVVFGGGTKLTVL |
| 289 | Anti CD122 | EVQLVQSGAEVKKPGSSVKVSCKASGYTFTNYYMHWVRQAPGQGLEWMGA |
| | clone P2C4_G2 | IMPSRGGTSYPQKFQGRVTMTGDTSTSTVYMELSSLRTEDTAVYYGARGE |
| | | YYYDSSGYYYWGQGTLVTVSSGGGGSGGGGSGGVGSQSALTQPASVSGSP |
| | | GQSIAISCTGTSSDIGHYDFVSWYQQHPGTAPKLIIYDINNRPSGISNRF |
| | | SGSKSDNMASLTISGLQPEDEADYYCSAYTSSDTVVFGGGTKLTVL |
| 290 | Anti CD122 | EVQLVQSGAEVKKPGSSVKVSCKASGYTFTNYYMHWVRQAPGQGLEWMGA |
| | clone P2C4_G11 | IMPSRGGYSYPQKFQGRVTMTGLTSYSTVYMELSNLRSEDTAVYYCARGE |
| | | YYYDSSGYYYWGQGTLVTVSSGGGGSGGGGSGGGGSQSALTQPASVSGSP |
| | | GQSIAISCTGTSSDIGHYDFVSWYQQHPGTAPKLIIYDINNRPSGISNRF |
| | | SGSKSQNMASLTISGLQPEDEADYYCSAYTSSDTVYFGGGTKLTVL |
| 291 | Anti CD122 | EVQLVQSGAEVKKPGSSVKVSCKASGYTFTNYYMHWVRQAPGQGLEWMGA |
| | clone P2C4_H1 | IMPSRGGTSYPQKFQGRVIMIGDISTSIVYMELSSLRSEDTAVYYCARGE |
| | | YYYDISSGYYYWGQGTLVNVSSGGGGSGGGGSGGGGSQSALTQPASVSGS |
| | | PGQSIAISCTGTSSDIGHYDEVSWYQQHPGTAPKLIIYDINNRPSGISNR |
| | | FSGSKSQNMASLTISGLQPEDEADYYCSAYTSSDTVVFGGGTKLTVL |
| 292 | Anti CD122 | EVQLVQSGAEVKKPGSSVKVSCKASGYTFSNYYMHWVRQAPGQGLEWGAI |
| | clone P2C4_H2 | MPSRGGTSYPQKFQGRVTMTGDTSTSTVYMELSSLRSEDTAVYYCARGEY |
| | | YYDSSGYYYWGQGTLVTVSSGGGGSGGGGSGGGGSQSALTQPASVSGSPG |
| | | QSIAISCTGTSSDIGHYDEVSWYQQHPGTAPKLIIYDINNRPSGISNRFS |
| | | GSKSDNMASLTISGLQPEDEADYYCSAYTSSDTVVFGGGTKLTVL |
| 293 | Anti CD122 | EVQLVQSGAEVKKPGSSVKVSCKATGYTFTNYYMHWVRQAPGQGLEWMGA |
| | clone P2C4_H3 | IMPSRGGYSYPQKFQGRVTMTGQTSYSTVYMELSSLRSEDTAVYYCARGE |
| | | YYYDSSGYYYWGQGTLVTVSSGGGGSGGGGSGGGGSQSALTQPASVSGSP |
| | | GQSIAISCTGTSSDIGHYDFVSWYQQHPGTAPKLIIYDINNRPSGISNRF |
| | | SGSKSQNMASLTISGLQPEDEADYYCSAYTSSDTVVFGGGTKLTVL |
| 294 | Anti CD122 | EVQLVQSGAEVKKPGSSYKVSCKASGYTFTNYYMHWVRQAPGQGLEWMGA |
| | clone P2C4_C1D10 | IMPSRGGTSYPQKFQGRVTMTGDYSTSIVYMELSSLRSEDTAVYYCARGE |
| | | YYYDSSGYYYWGQGTPVTVSSGGGGSGGGGSGGGGSQSALTQPASVSGSP |
| | | GQSIAISCTGTSSDIGDYDFVSWYQQHPGTAPKLIIYDINNRPSGISNRF |
| | | SGSKSQNMASLTISGLQPEDEADYYCSAYTSSDTVVFGGGTKLTVL |
| 295 | Anti CD122 | EVQLVQSGAEVKKPGASYKVSCKASGYTFTNYYMHWVRQAPGQGLEWMGA |
| | clone P2C4_FW2 | IMPSRGGTSYPQKFQGRVTITADKSTSTAYMELSSLRSEDTAVYYCARGE |
| | | YYYDSSGYYYWGQGTLVTVSSGGGGSGGGGSGGGGSQSVLTQPPSVSGAP |
| | | GQRVTISCTGTSSDIGHYDFVSWYQQLPGTAPKLLIYDINNRPSGVPDRE |
| | | SGSKSGTSASLAITGLQAEDEADYYCSAYTSSDTLVFGGGTKLTVL |
| 296 | Anti CD122 | EVQLVQSGGGVVQPGRSLRLSCAASGFTFSSYAMHWVRQAPGKGLEWVAV |
| | clone P1E7 | ISYDGSNKYYADSVKGRFTISRQNSKNTLYLQMNSLRAEDTAVYYCARDL |
| | | GYSSSWYYYYYGMDVWYGQGTTVTVSSGGGGSGGGGSGGGGSDVVMTQSP |
| | | LSLPVTPGEPASISCRSSQSLLHSNGYNYLDWYLQKPGQSPQLLIYLGSS |
| | | RASGVPQRFSGSGTDFTLKISRVEAEDVGVYYCMQALQTPRTFGQGTKLE |
| | | IK |
| 297 | Anti CD122 | QVQLQESGPGLVKPSETLSLTCTYSGYSISSRSDHWGWVRQPPGKGLEWI |
| | clone P1B10 | GSISYSGSTYYNPSLKSRVTISVDISKNQLSLKLSSVTAADTAVYYCARE |
| | | SHPAAALYGWGQGTLVTVSSGGGGSGGGGSGGGGSDIQMTQSPSSLSASV |
| | | GDRVTITCQASQDISDYLNWYQQKPGKAPQILIYDASNLETGVPSRFSGS |
| | | GSGTDFTFTISNLQPEDVATYYCQQYEDLPSFGGGTKVEIK |
| 298 | Anti CD122 | EVQLVQSGGGLVQPGGSLRLSCAASGFTFSSYAMSWVRQAPGKGLEWVSA |
| | clone P1F3 | ISGSGGSTHYADSVKGRFTISRQNSKNTLYLQMNSLRAEDTAVYYCATPA |
| | | FWGQGTLVTVSSGGGGSGGGGSGGGGSDIQLTQSPSSLSASVGDRVTITC |
| | | RASQSISSYLNWYQQKPGKAPKALIYDASNLETGVPSRFSGSGSGTDFTL |
| | | TIISLQPEDFATYFCLQDYTYPWTFGQGTKVEFK |
| 299 | Anti CD122 | QVQLQQWGPGLVKPSETLSLTCTVSGGSISSYYWSWIRQPPGKGLEWIGE |
| | clone P1D10 | INHSGSTNYNPSLKSRVTISVDTSKNQFSLKLSSVTAADTAVYYCAGGEN |
| | | LQWFDPWGQGYLVTVSSGGGGSGGGGSGGGGSQSVLTQPPSVSGAPGQRV |
| | | TISCTGGSSNYGAGYDVHWYQQLPGTVPKLLIYDNTNRPSGVPDRESASK |
| | | SGTSASLVITGLQAEDEGDYYCQSYDSSLRASVFGGGTMLTVL |
| 300 | Anti CD122 | QVQLQQWGAGLLKPSETLSLTCAVYGGSFSGYYWSWIRQPPGKGLEWIGE |
| | clone P1E1 | INHSGSINYNPSLKSRVTISVDISKNQFSLELSSVTAADTAVYYCARADR |
| | | RFGELRYWGQGTLVTVSSGGGGSGGGGSGGGGSNFMLTQPHSVSESPGKT |
| | | VTISCTGSSGSIASSYVQWYQQRPGSAPTTMYADNQRPSGVPDRESGSVQ |
| | | SSSNSASLTISGLKTEDEADYYCQSFDSSLYMIFGGGTKLTVL |
| 301 | Anti CD122 | QVQLVQSGGGLVKPGGSLRLSCAASQFTFSSYDLHWVRQVPGRGLEWVSL |
| | clone P2B11 | ISYDGSNKYYADSVKGRFTISRQNAENSLYLQMNSLRAEDTAVYYCAREP |
| | | ITGTSDLFDYWGQGTLVTVSSGGGGSGGGGSGGGGSQSVLTQPPSVSGAP |
| | | GQRVTISCTGSRSNIGAGYDVHWYQHLPGTAPKLLIYQNSNRPSGVSDRF |
| | | SGSKSGTSASLAITGLQAEDEADYYCQSFQSSLRGVVFGGGTRLTVL |
| 302 | Anti CD122 | QVQLQESGPQLVKPSGTLSLTCAVSGGSISSSNWWSWVIRQPPGKGLEWI |
| | clone P2C9 | GEIYHSGSTNYNPSLKSRVTISVQKSKNQFSLKLSSVTAADTAVYYCVRE |
| | | GGLREEHWGQGTLVTVSSGGGGSGGGGSGGGGSSYELTQPPSÅSGTPGQR |
| | | VTISCSGSSSNIGSNTVNWYQQLPGTAPKLLIYSNNQRPSGVPQRFSGSK |
| | | SGTSASLAISGLQSEDEADYYCAAWDDSINGLWVFGGGTKLTVL |
| 303 | Anti CD122 | QVQLQQWGAGLLKPSETLSLTCAVYGGSFSGYYWSWIRQPPGKGLEWIGE |
| | clone P2C10 | INHSGSTNYNPSLKSRVTISVDISKNQFSLKLSSVTAADTAVYYCARGID |
| | | TAMADYWGQGTLVTVSSGGGGSGGGGSGGGGSDVVMTQSPLSLPVTPGEP |
| | | ASISCRSSQRLLHSNGYNYVDWYLQKPGQSPQLLIYLGSNRASGYPQRFS |
| | | GSGSGTQFILKISRVEAEDVGVYYCMQGTHWPWTFGQGIKVEIK |
| 304 | Anti CD122 | QVQLQQSGPGLVKPSQTLSLTCAISGDSVSGNSATWNWIRQSPSRGLEWL |
| | clone P2C11 | GRTYYRSKWNHDYAESVKSRITINPDYSKNQFSLQLNSVTPEDTAVYYCA |
| | | RDSKSAFDIWGQGYMVTVSSGGGGSGGGGSGGGGSDIQMTQSPPSLSASV |
| | | GDRVTITCQASQUINNYLNWYHQKPGKAPELLIYDASQLETGVPSRFSGS |
| | | GSGTEFTFIISSLQPEDTGTYYCQQYDWLPLSYGGGTKVEIK |
| 305 | Anti CD122 | QLQLQESGPGLVKPSETLSLTCSVFGVSITSGSWWSWVRQSPGKELEWIG |
| | clone P2ES | EIYHNGNTNYNPSLKSRVTISVQTSKNQFSLKLSSVTAADTAVYYCVSGF |
| | | DYWGQGTLVTVSSGGGGSGGGGSGGGGSNFMLTQPHSVSGSPGKTITISC |
| | | TRSSGNFASTYVQWYQQRPGSSPAIVIYDDDQRPSGVPQRFSGSIDRSSN |
| | | SASLTISGLETEDEADYYCQSYDSBNFWVFGGGTKLTVL |
| 308 | Anti CD122 | QVQLQESGPGLVKPSETLSLTCTVSGVSISSRSDHWGWVRQPPGKGLEWI |
| | clone P2E11 | GSISYSGSTYYNPSLKSRVTISVDTSKNQLSLKLSSVTAADTAVYYCARE |
| | | SHPAÅALVGWGQGTLVTVSSGGGGSGGGGSGGGGSEIVLTQSPSSLSASV |
| | | GDRVTITCQASQDINNYLNWYQQKPGKAPKLLIYDASNLETGVPSKFSGS |
| | | GSGTDFTFTISSLQPEDIATYYCQQYANLPSFGQGTKLEIK |
| 307 | Anti CD122 | EVQLVQSGAEVKKPGSSVKVSCKASGYTFTSYGISWVRQAPGQGLEWMGW |
| | clone P2F9 | ISAYNGNTNYAQKLQGRVTMTTDTSTSTAYMELSSLRSEDTAVYYCARAP |
| | | DYGDSSNYYYYYMDVWGKGTTVTVSSGGGGSGGGGSGGGGSEIVLTQSPL |
| | | SLPVTPGEPASISCRSSQSLLHSNGYNYLDWYLQKPGQSPQLLIYLGSNR |
| | | ASGVPQRFSGSGSGTDFILSISRVEAEDVGVYYCMQALQTPPTFGQGTKV |
| | | EIK |
| 308 | Anti CD122 | QVQLVQSGAEVKKPGASYKVSCKASGYIFTSYGISWVRQAPGQGLEWMGW |
| | clone P2F10 | ISAYNGNTNYAQKLQGRVTMITDYSTSTAYMELRSLRSDQTAVYYCARDT |
| | | SGDYSSGWYLGVPFDYWGQGTLVTVSSGGGGSGGGGSGGGGSDIQLTQSP |
| | | SSLSASVGDRVTVTCQASQDIGHNLNWYQQRPGKAPQLLIYDASNLETGV |
| | | PSRFSGSGSGTQFTFTISSLQPEDIATYYCQQYDELPPDEGPGTKVEIK |
| 309 | Asil CD132 | QVQLQQWGAGLLKPSETLSLTCAVYGGSFSGYYWSWIRQPPGKGLEWIGE |
| | clone P1A3 | INHSGSINYNPSLKSRATISVDYSKNQFSLKLSSVTAADTAVYYCATSPG |
| | | GYSGGYFQHWGQGTLVTVSSGGGGSGGGGSGGGGSDVVMTQSPLSLPVTP |
| | | GEPASISCRSSQSLLHSNGYNYLDWYLQKPGQSPQLUYLGSNRDSGVPDR |
| | | FSGSGSGTDFTLKISRVEAEDVGVYYCMQGTHWPWTFGQGTKVEIKNSGA |
| | | GTAAATHTCPPCPAPEAAGGPSVFLFPPKPKDTLMISRTPEVTCVVVDVS |
| | | HEDPEVKFNWYVDGVEVHNAKTKPREEQYNSTYRVVSVLTVLHQDWLNGK |
| | | EYKCKVSNKALPAPIEKTISKAKGQPREPQVCTLPPSRDELTKNQVSLSC |
| | | AVKGFYPSDIAVEWESNGQPENNYKTTPPVLDSDGSFFLCVSKLTVDKSR |
| | | WQQGNVFSCSVMHEALHNHYTQKSLSLSPGK |
| 310 | Anti CD132 | QVQLQESGPGLVKPSETLSLTCTVSCGGSISSSSYYWIRQPPGKGLEWIG |
| | clone P2B9 | SIYYSGSTYYNPSLKSRVIISVDTSKNQFSLKLSSVTAAUTAVYYCAGDI |
| | | LTGYALDYWGQGILVTVSSGGGGSGGGGSGGGGSSYELTQPPSMSVSPGQ |
| | | TARITCSGDALPKQFAFWYQQKPGQAPVLVIYKDTERPSGIPERFSGSSS |
| | | GTTVTLTITGVQAEDEADYYCQSPDSSGTVEVFGGGTKLTVL |
| 311 | Anti CD132 | QVQLQQWGAGLLKPSETLSLTCAVYGGSFSGYYWSWIRQPPGKGLEWIGE |
| | clone P1A3_B3 | INHFGSTNYNPSLKSRATISVQTSKNQFSLKLSSVTAADTAVYYCATSPG |
| | | GYSGGYFQHWGQGTLVT |
| | | VSSGGGGSGGGGSGGGGSDVVMTQSPLSLPVTPGEPASISCRSSQSLLHS |
| | | NGYNYLDWYLQKPGQSPQLLIYLGSNRDSGVPDRFSGSGSGTDFTLKISR |
| | | VEAEDYGVYYCMQGTHWPWTFGQGTKVEIK |
| 312 | Anti CD132 | QVQLQQWGAGLLKPSETLSLTCAVYGGSFSGYYWSWIRQPPGKGLEWIGE |
| | clone P1A3_B4 | INHFGSTNYNPSLKSRATISVDTSKNQFSLKLSSVTAADTAVYYCATSPG |
| | | GYFGGYFQHWGQGYLVTVSSGGGGSGGGGSGGGGSDVVMTQSPLSLPVTP |
| | | GESVSISCRSSQSLLHSNGYNYLDWYLQKPGQSPQLLIYLGSNRDSGVPQ |
| | | RFSGSGSGTDFTLKISRVEAEDVGVYYCMQGTHWPWTFGQGTKVEIK |
| 313 | Anti CD132 | QVQLQQWGAGMLKPSEILSLTCAVYGGSFSGYYWSWIRQPPGKGLEWIGE |
| | clone P1A3_E8 | INHFGSTNYNPSLKSRATISVDTSKNQFSLKLSSVTAADTAVYYCATSPG |
| | | GYSGGYFQHWGQGTLVTVSSGGGGSGGGGSGGGGSDVVMTQSPLSLPVTP |
| | | GEPASISCRSSQSLLHSNGYNYLDWYLQKPGQSPQLLIYLGSNRDSGVPD |
| | | RFSGSGSGTDFTLKISRVEAEDVGVYYCMQGTHWPWTFGQGTKVEIK |
| 314 | Anti CD132 | QVQLQQWGAGLLKPSETLSLTCAVYGGSFSGYYWSWIRQPPGKGLEWIGE |
| | clone P1A3_E9 | INHFGSTNYNPSLKSRATISVDTSKNQFSLKLSSVTAADTAVYYCATSPG |
| | | GYSGGYFQHWGQGTLVTVSSGGGGSGEGGSGGGGSDVVMTQSPLSLPVTP |
| | | GEPASISCRSSQSLLHSNGYNYLDWYLQKPGQSPQLLIYLGSNRDSGVPD |
| | | RFSGSGSGTDFTLKISRVEAEDVGVYYCMQGTHWPWTFGQGTKVEIKAAA |
| | | HHHHH |
| 315 | Anti CD132 | EVQLVESGGGLVQPGGSLRLSCAASGGSFSGYYWSWVPQAPGKGLWVSEI |
| | clone P1A3_FW2 | NHSGSTNYNPSLKSRFTISRDNSKNTLYLQMNSLRAEDTAVYYCARSPGG |
| | | YSGGYFQHWGQGILVTVSSGGGGSGGGGSGGGGSDIQMTQSPSSLSASVG |
| | | DRVTITCRSSQSLLHSNGYNYLDWYQQKPGKAPKLLIYLGSNRDSGVPSR |
| | | FSGSGSGTDFTLTISSLQPEDFATYYCMQGTHWPWTFGQGTKVEIK |
| 316 | Anti CD132 | QVQLQQSGAEVKKPGSSVKVSCKASGGTFSSYAISWVRQAPGKGLEWMGG |
| | clone P1A10 | FDPEDGETIYAQKFQGRVTMTEDTSTDTAYMELSSLRSEDTAVYYCATDL |
| | | RIPYYYDNPWGQGTLVTVSSGGGGSGGGGSGGGGSEIVLTQSPLSLPVTP |
| | | GEPASISCRSSQSLLHSNGYNYLNWYLQKPGQSPQLLIYLGSDRASGVPD |
| | | RFSGSGSGTDFTLKISRVEAEDVGVYYCMQALQTPTTEGGGTKVEIK |
| 317 | Anti CD132 | QVQLVQSGGGVVQPGRSLRLSCAASGFTFSSYAMHWVRQAPGKGLEWVAV |
| | clone P1B6 | ISYDGSNKYYADSVKGRETISRDNSKNTLYLQMNSLRAEDTAVYYCARSL |
| | | YYSHFDYWGQGTLVTVSSGGGGSGGGGSGGGGSEIVMIQSPLSLPVTPGE |
| | | PASISCRSSQSLLHSNGYNYLDWYLQKPGQSPQLLMYLVSNRASGVPERF |
| | | SGSGSGTDFTLKISRVEAEDVGVYYCMQTLQTPLSFGQGTKLEIK |
| 318 | Anti CD132 | EVQLVETGPGLVKPSGTLSLTCAVSGGSISSENWWSWVRQPPGKGLEWIG |
| | clone P1C10 | EIYHSGSTNYNPSLKSRVTISVQKSKNQFSLKLSSVTAADTAVYYCAREG |
| | | PLSSSGPGAFDIWGQGTMVTVSSGGGGSGGGGSGGGGSEIVLTQSPATLS |
| | | LSPGERATLSCRASGSVSYHLAWYQQKPGGAPRLLIYQDTSNRASGIPAR |
| | | FSGSGSGTDFTLTINSLEPEDFAVYYCQQRYDWPLTFGGGTKVEIK |
| 319 | Anti CD132 | QVQLQESGGGVVQPGRSLRLSCAASGFTFSNYGMHWVRQAPGKGLEWVAV |
| | clone P1D7 | ISYDGTNKYYADSVKGRFTISRQNSKNTVYLQMNSLRAEDTAVYYCAKDG |
| | | FDIWGQGTMVTVSSGGGGSGGGGSGGGGSDIQMTQSPSFLSASVGDRVTI |
| | | TCRASQSISSWLAWYQQKPGKAPKLLIYDASRLEDGVPSRFSGTGFGTDF |
| | | TFTITTLQPDDIATYYCQQYDDLPYTFGQGTTVDIK |
| 320 | Anti CD132 | EVQLVQSGGGVVQPGRSLRLSCAASGFTFSSYGMHWVRQAPGKGLEWVAV |
| | clone P1E8 | ISYDGSNKYYADSVKGRFTISRDNSKNTLYLQMNSLRAEDTAVYYCARDV |
| | | YGDYGAFDYWGQGTLVTVSSGGGGSGGGGSGGGGSDVVMTQSPVSLPVTL |
| | | GQPASISCKSSQSLLYENGNTYLSWFQQRPGQSPRRLFYQVSNRDSGVPD |
| | | RFSGSGSDTDFTLTISRVEAEDVGVYFCMQGTQWPPTFGQGTKVEIK |
| 321 | Anti CD132 | QLQLQESGGGVVQPGRSLRLSCAASGFTPSSYAMHWVRQAPGKGLEWVAV |
| | clone P2B2 | ISYDGGNKYYADSVKGRETISRDNSKNTLYLQMNSLRAEDTAVYYCAKSV |
| | | APPMDVWGKGTYVTVSSGGGGSGGGGSGGGGSDVVMTQSPLSLPVTPGEP |
| | | ASISCRSSQSLLHENGYNYLDWYLQKPGQSPHLLIYLGSNRASGVPDRFS |
| | | GSGSGTDFTLKISRVEAEDVGVYFCMQALRTPYTFGQGTKLEIK |
| 322 | Anti CD132 | QVQLQQWGAGLLKPSETLSLTCAVYGESFSGYYWSWIRQPPGKGLEWIGE |
| | clone P2B7 | INHSGSTNYNPSLKSRVTISVDYSKNQFSLKLSSVTAADTAVYYCARGPA |
| | | GSSSSGYFDYWGQGTLVTVSSGGGGSGGGGSGGGGSDVVMTQSPLSLPVT |
| | | LGQPASISCRSSQSLVHSNGYNYLDWYLQKPGQSPQLLIYLGSNRASGVP |
| | | DRFSGSGSGTDFTLKISRVEAEDVGVYYCLQGSHWPWTFGQGTKVEIK |
| 323 | Anti CD132 | QVQLQESGPGLVKPSQTLSLTCTVSGGBISSGGYYWTWIRQHPGQGLEWI |
| | clone P2D11 | GFISWSGTTYYNPSLKNRVTISADTSKNHFSLNLTSVTAADTAVYYCARG |
| | | SGRLVWGQGTLVTVSSGGGGSGGGGSGGGGSETTLTQSPATLSVSPGERA |
| | | TESCRASQSVSSNLAWYQQKPGQAPRLLIYGASSGATGIPDRFSGSGSGT |
| | | DFTLTISRLEPEDFAVYYCQLYGSSLAFGGGTKVEIK |
| 324 | Anti CD132 | EVQLVQSGAEVKKPGASVKVSCKASGYTFTGYYMHWVRQAPGQGLEWMGI |
| | clone P2F10 | INPSGGSTSYAQKFQGRVTMTRDTSTSTVYMELSSLRSEDTAVYYCARAD |
| | | TAMGDAFDIWGQGTMVTVSSGGGGSGGGGSGGGGSDIVMTHTPLSLPVTP |
| | | GEPASISCRSSQTLFDSDDGKTYLDWYLQKPGQSPQLLMYTTBSRASGVP |
| | | DRFSGSGSGTDFTLKISRVEAEDVGVYYCMQRLQFPLIFGQGIRLEFK |
| 325 | Anti CD132 | EVQLVQSGGGVVQPGRSLRLSCAASGFTFSSYAMHWVRQAPGKGLEWVAV |
| | clone P2H4 | ISYDGSN |
| | | KYYADSVKGRFTISRDNSKNTLYLQMNSLRAEDTAVYYCARSIGIGAFDI |
| | | WGQGTMVTVSSGGGGSGGGGSGGGGSDVVMTQSPLSLPVTPGEPASISCR |
| | | ATQSLLHGNGHNYLDWYLQKPGQSPQLLIYLGSNRASGVPDRFSGSGSGT |
| | | DFTLKISRVEAEDVGVYYCMQTLETPVTFGPGTKVDIK |
| 326 | Anti CD132 | QVQLQQWGAGLLKPSETLSLTCTIYGGSFSGFYWSWIRQPPGKGLEWIGE |
| | clone P2D3 | INHSGSTNYNPSLKSRVTISVDTSKNQFSLKLSSVTAADTAIYYCARGPA |
| | | GSTSSGYFDHWGQGTLVTVSSGGGGSGGGGSGGGGSDVVMTQSPLSLPVT |
| | | PGEPASISCRSSQSLLHSNGYNYLDWYLQKPGQSPQLLIYLGSNRASGVP |
| | | DRFSGSGSGTDFTLKISRVEAEDVGVYYCMQGTHWPWTFGQGTKVEIK |
| 327 | Anti CD132 | QVQLQQWGAGLLKPSETLSLTCAVYGGSLSGYYWSWIRQPPGKGLEWIGE |
| | clone P1G4 | INHSGSTNYNPSLKSRYTISVDTSKNQFSLKLSSVTAADTAVYYCARGSS |
| | | BYYMDVWGKGTTVTVSSGGGGSGGGGSGGGGSDVVMTQSPLSLPVTPGEP |
| | | ASISCRSSQSLLHSNGYNYLDWYLQKPGQSPQLLIYLGSNRASGVPDRFS |
| | | GSGSGTDFTLKISRVEAEDVGVYYCLQGTHWPWTFGQGTKVEIK |
| 328 | Anti CD132 | QVQLQQWGAGLLKPSETLSLTCAVYGGSFSGYYWSWIRQPPGKGLEWIGE |
| | clone P1B12 | INHSGSINYNPSLKSRVTISVDTSKNQFSLKLSSVTAADTAVYYCARGGS |
| | | AYFQHWGQGTLVTVSSGGGGSGGGGSGGGGSDVVMTQSPLSLPVTLGQPA |
| | | SISCRSSQSLLHSNGNNYLDWYLQKPGQSPQLLIYLGSNRASGVPDRFSG |
| | | SGSGTQFTLKISRVEAEDYGIYYCMQGTHWPWTEGQGTKVEIE |
| 329 | Ant: CD132 | QVQLQQWGAGLLKPSETLSLTCAVYGGSFSGYYWSWIRQPPGKGLEWIGE |
| | clone P1G7 | INHSGSTNYNPSLKSRVTISEDASKKQFSLTLTSVTAADTAVYYCARGPA |
| | | GTGSSGYFDYWGQGTLVTVSSGGGGSGGGGSGGGGSEIVLTQSPLSLPVT |
| | | PGEPASISCRSSQSLLHSNGYNYLDWYLQKPGQSPQLLIYLASNRASGVP |
| | | DRPSGSGSGTDFTLKISRVEAEDVGVYYCMQGTHWPWTFGQGTKVEVK |
| 330 | Linker 1 | NSGAGTAAA |
| 331 | Linker 2 | NSGAGTSGSGASGEGSGSKLAAA |
| 332 | Linker 3 | GGGGSAAA |
| 333 | Linker 4 | GGGGSGGGGSGGGGS |
| 334 | Tag | AAAHHHHHH |
| 335 | Anti CD122 | CAGTCTGCCCTGACTCAGCCTGCCTCCGTGTCTGGGTCTCCTGGACAGTC |
| | P2C4 Fab LC | GATCGCCATTTCCTGCACTGGAACCAGCAGTGACATTGGTCATTATGACT |
| | (VL, johnt CL) | TTGTCTCCTGGTACCAACAGCACCCAGGCACAGCCCCCAAACTCATAATT |
| | | TATGATATCAATAATCGGCCCTCAGGGATTTCTAATCGCTTCTCTGGCTC |
| | | CAAGTCTGACAATATGGCCTCCCTGACCATCTCTGGGCTCCAGCCTGAGG |
| | | ACGAGGCTGATTATTACTGCAGTGCATATACAAGCAGCGACACTCTGGTC |
| | | TTCGGCGGAGGGACCAAGTTGACCGTCCTCAGTCAGCCCAAGGCTGCCCC |
| | | CTCGGTCACTCTGTTCCCACCCTCCTCTGAGGAGCTTCAAGCCAACAAGG |
| | | CCACACTGGTGTGTCTCATAAGTGACTTCTACCCGGGAGCCGTGACAGTG |
| | | GCCTGGAAGGCAGATAGCAGCCCCGTCAAGGGGGGAGTGGAGACCACCAC |
| | | ACCCTCCAAACAAAGCAACAACAAGTACCCGGCCAGCAGCTACCTGAGCC |
| | | TGACGCCTGAGCAGTGGAAGTCCCACAAAAGCTACAGCTGCCAGGTCACG |
| | | CATGAAGGGAGCACCGTGGAGAAGACAGTGGCCCCTACAGAATGTTCA |
| 336 | Anti CD122 P2C4 | GAGGTCCAGCTGGTACAGTCTGGGGCTGAGGTGAAGAAGCCTGGGTCCTC |
| | Fab HC | AGTGAAGGTTTCCTGCAAGGCATCTGGATACACCTTCACCAACTACTATA |
| | (VH, joint CH1) | TGCACTGGGTGCGACAGGCCCCTGGACAAGGGCTTGAGTGGATGGGGGCA |
| | | ATCATGCCTAGTCGTGGTGGCACAAGTTACCCACAGAAGTTCCAGCGCAG |
| | | AGTCACCATGACCGGGGACACGTCCACGAGCACAGTCTACATGGAGCTGA |
| | | GCAGCCTGAGATCTGAGGACACGGCCGTGTATTACTGTGCGAGAGGGGAG |
| | | TATTACTATGATAGTAGTGGTTATTACTACTGGGGCCAGGGCACCCTGGT |
| | | CACCGTCTCAAGCGCCTCCACCAAGGGCCCATCGGTCTTCCCCCTGGCAC |
| | | CCTCCTCCAAGAGCACCTCTGGGGGCACAGCGGCCCTGGGCTGCCTGGTC |
| | | AAGGACTACTTCCCCGAACCGGTGACGGTGTCCTGGAACTCAGGCGCCCT |
| | | GACCAGCGGCGTCCACACCTTCCCGGCTGTCCTACAGTCCTCAGGACTCT |
| | | ACTCCCTGAGCAGCGTAGTGACCGTGCCCTCCAGCAGCTTGGGCACCCAG |
| | | ACCTACATCTGCAACGTGAATCACAAGCCCAGCAACACCAAGGTGGACAA |
| | | GAAAGTTGAGCCCAAATCTTGT |
| 337 | Anti CD122 P2C4 | AGGTCTCCTGTAAAGCAAGCGGATACACATTCACAAACTACTATATGCAC |
| | scFv and Fc | TGGGTGCGGCAGGCCCCCGGACAGGGCCTGGAGTGGATGGGGGCTATCAT |
| | with knob | GCCTTCCCGAGGCGGGACTTCTTACCCACAGAAGTTCCAGGGAAGAGTGA |
| | modification | CCATGACAGGCGACACTAGCACCTCCACAGTCTATATGGAGCTGAGCAGC |
| | | CTGAGGAGCGAAGACACTGCCGTGTACTATTGCGCTCGCGGAGAATACTA |
| | | TTACGATTCTAGTGGCTATTACTATTGGGGGCAGGGAACACTGGTGACTG |
| | | TCTCAAGCGGAGGAGGAGGAAGTGGCGGAGGAGGCTCCGGAGGAGGCGGG |
| | | TCTCAGAGTGCACTGACCCAGCCAGCATCAGTGAGCGGCAGCCCCGGCCA |
| | | GTCTATCGCAATTAGTTGTACTGGGACCTCCTCTGACATCGGACACTACG |
| | | ATTTCGTCTCTTGGTATCAGCAGCACCCCGGCACCGCTCCTAAGCTGATC |
| | | ATCTACGACATCAACAATCGGCCCAGGGGCATTTCCAACAGATTTTCTGG |
| | | GAGTAAATCAGATAATATGGCCTCACTGACAATTAGCGGCCTCCAGCCTG |
| | | AGGACGAAGCTGATTACTATTGCTCCGCATACACTAGTTCAGATACCCTG |
| | | GTGTTTGGAGGCGGGACCAAACTGACAGTCCTGAACAGCGGCGCGGGCAC |
| | | CGCGGCCGCGACTCACACATGCCCACCGTGCCCAGCACCTGAAGCCGCGG |
| | | GGGGACCGTCAGTCTTCCTCTTCCCCCCAAAACCCAA |
| | | GGACACCCTCATGATCTCCCGGACCCCTGAGGTCACATGCGTGGTGGTGG |
| | | ACGTGAGCCAGGAAGACCCTGAGGTCAAGTTCAACTGGTACGTGGACGGC |
| | | GTGGAGGTGCATAATGCCAAGACAAAGCCGGGGGAGGAGCAGTACAACAG |
| | | CACGTACCGTGTGGTCAGCGTCCTCACCGTCCTGCACCAGGACTGGCTGA |
| | | ATGGCAAGGAGTACAAGTGCAAGGTCTCCAACAAAGCCCTCCCAGCCCCC |
| | | ATCGAGAAAACCATCTCCAAAGCCAAAGGGCAGCCCCGAGAACCACAGGT |
| | | GTACACCCTGCCCCCATGCCGGGATGAGCTGACCAAGAACCAGGTCAGCC |
| | | TGTGGTGCCTGGTCAAAGGCTTCTATCCCAGCGACATCGCCGTGGAGTGG |
| | | GAGAGCAATGGGCAGCCGGAGAACAACTACAAGACCACGCCTCCCGTGCT |
| | | GGACTCCGACGGCTCCTTCTTCCTCTACAGCAAGCTCACCGTGGACAAGA |
| | | GCAGGTGGCAGCAGGGGAACGTCTTCTCATGCTCCGTGATGCATGAGGCT |
| | | CTGCACAACCACTACACGCAGAAGAGCCTCTCCCTGTCTCCGGGTAAA |
| 338 | Anti CD122 P2H7 Fab | GACATCCAGATGACCCAGTCTCCTTCCACATTGTCTGCATCTGTAGGAGA |
| | LC (VL, joint CL) | CAGAGTCACACTCTCTTGCCGGGCCGGTCAGGCTATTAGTAGTTGGTTGG |
| | | CCTGGTATCAACAGAAACCAGGTAAAGCCCCAAAGCTTCTGATCTATAAG |
| | | GCATCTAATTTAGAAAGTGGAGTCCCATCAAGGTTCAGCGGCGGTGGATC |
| | | TGGGGCAGAATTCACTCTCACCATCAGCAGCCTGCAGCCTGATGATTTTG |
| | | CAACTTATTACTGCCAACAGTATCAGAGCTACCCTTACACTTTTGGCCAG |
| | | GGGACCAAGCTGGAGATCAGACGAACTGTGGCTGCACCATCTGTCTTCAT |
| | | CTTCCCGCCATCTGATGAGCAGTTGAAATCTGGAACTGCCTCTGTTGTGT |
| | | GCCTGCTGAATAACTTCTATCCCAGAGAGGCCAAAGTACAGTGGAAGGTG |
| | | GATAACGCCCTCCAATCGGGTAACTCCCAGGAGAGTGTCACAGAGCAGGA |
| | | CAGCAAGGACAGCACCTACAGCCTCAGCAGCACCCTGACGCTGAGCAAAG |
| | | CAGACTACGAGAAACACAAAGTCTACGCCTGCGAAGTCACCCATCAGGGC |
| | | CTGAGCTCGCCCGTCACAAAGAGCTTCAACAGGGGAGAGTGT |
| 339 | Anti CD122 P2H7 Fab | GAGGTGCAGCTGGTGCAGTCTGGGACTGAGGTGAAGAAGCCTGGGGCCTG |
| | HC (VH, joint CH1) | AGTGAAGGTTTCCTGCAAGGCTTCTGGATACACCTTCACTACCTATGCTA |
| | | TGCATTGGGTGCGCCAGGCCCCCGGACAAAGCCTTGAGTGGATGGGATGG |
| | | ATCAACACTGGCAATGGTAACACAAAATATTCACAGAACTTCCAGGGCAG |
| | | AGTCACCATGACCAGGGACACGTCCATCAGCACAGCCTACATGGAGCTGA |
| | | GCAGGCTGAGATCTGACGACACGGCCGTGTATTACTGTGCGAGAGATCTC |
| | | GGGCAACTGGAACGACTCTACTTCTGGGGCCAGGGCACCCTGGTCACCGT |
| | | CTCAAGCGCCTCCACCAAGGGCCCATCGGTCTTCCCCCTGGCACCCTCCT |
| | | CCAAGAGCACCTCTGGGGGCACAGCGGCCCTGGGCTGCCTGGTCAAGGAC |
| | | TACTTCCCCGAACCGGTGACGGTGTCGTGGAACTCAGGCGCCCTGACCAG |
| | | CGGCGTCCACACCTTCCCGGCTGTCCTACAGTCCTCAGGACTCTACTCCC |
| | | TCAGCAGCGTAGTGACCGTGCCCTCCAGCAGCTTGGGCACCCAGACCTAC |
| | | ATCTGCAACGTGAATCACAAGCCCAGCAACACCAAGGTGGACAAGAAAGT |
| | | TGAGCCCAAATCTTGT |
| 340 | Anti CD122 P2H7 scFv | GAGGTGCAGCTGGTGCAGTCTGGGACTGAGGTGAAGAAGCCTGGGGCCTC |
| | and Fc with knob | AGTGAAGGTTTCCTGCAAGGCTTCTGGATACACCTTCACTACCTATGCTA |
| | modification | TGCATTGGGTGCGCCAGGCCCCCGGACAAAGCCTTGAGTGGATGGGATGG |
| | | ATCAACACTGGCAATGGTAACACAAAATATTCACAGAACTTCCAGGGCAG |
| | | AGTCACCATGACCAGGGACACGTCCATCAGCACAGCCTACATGGAGCTGA |
| | | GCAGGCTGAGATCTGACGACACGGCCGTGTATTACTGTGCGAGAGATCTC |
| | | GGGCAACTGGAACGACTCTACTTCTGGGGCCAGGGCACCCTGGTCACCGT |
| | | CTCAAGGGGAGGAGGAGGATCTGGCGGAGGAGGCAGTGGAGGAGGAGGGT |
| | | CACTTGACATCCAGATGACCCAGTCTCCTTCCACATTGTCTGCATCTGTA |
| | | GGAGACAGAGTCACACTCTCTTGCCGGGCCGGTCAGGCTATTAGTAGTTG |
| | | GTTGGCCTGGTATCAACAGAAACCAGGTAAAGCCCCAAAGCTTCTGATCT |
| | | ATAAGGCATCTAATTTAGAAAGTGGAGTCCCATCAAGGTTCAGCGGCGGT |
| | | GGATCTGGGGCAGAATTCACTCTCACCATCAGCAGCCTGCAGCCTGATGA |
| | | TTTTGCAACTTATTACTGCCAACAGTATCAGAGCTACCCTTACACTTTTG |
| | | GCCAGGGGACCAAGCTGGAGATCAGAAACAGCGGCGCGGGCACCGCGGCC |
| | | GCGACTCACACATGCCCACCGTGCCCAGCACCTGAAGCCGCGGGGGGACC |
| | | GTCAGTCTTCCTCTTCCCCCCAAAACCCAAGGACACCCTCATGATCTCCC |
| | | GGACCCCTGAGGTCACATGCGTGGTGGTGGACGTGAGCCACGAAGACCCT |
| | | GAGGTCAAGTTCAACTGGTACGTGGACGGCGTGGAGGTGCATAATGCCAA |
| | | GACAAAGCCGCGGGAGGAGCAGTACAACAGCACGTACCGTGTGGTCAGCG |
| | | TCCTCACCGTCCTGCACCAGGACTGGCTGAATGGCAAGGAGTACAAGTGC |
| | | AAGGTCTCCAACAAAGCCCTCCCAGCCCCCATCGAGAAAACCATCTCCAA |
| | | AGCCAAAGGGCAGCCCCGAGAACCACAGGTGTACACCCTGCCCCCATGCC |
| | | GGGATGAGCTGACCAAGAACCAGGTCAGCCTGTGGTGCCTGGTCAAAGGG |
| | | TTCTATCCCAGCGACATCGCGGTGGAGTGGGAGAGCAATGGGCAGCCGGA |
| | | GAACAACTACAAGACCACGCCTCCCGTGCTGGACTCCGACGGCTCCTTCT |
| | | TCCTCTACAGCAAGCTCACCGTGGACAAGAGCAGGTGGCAGCAGGGGAAC |
| | | GTCTTCTCATGCTCCGTGATGCATGAGGCTCTGCACAACCACTACACGCA |
| | | GAAGAGCCTCTCCCTGTCTCCGGGTAAA |
| 341 | Anti CD122 P2D12 | GACATCCAGTTGACCCAGTCTCCATCCTCCCTGTCTGCATGTGTAGGAGA |
| | Fab LC | CAGAGTCACCATCACTTGCCAGGCGAGTCAGGACATTGGCAACTATTTAA |
| | (VL, joint CL) | ATTGGTATCAGCTTAAACCAGGGAAAGCCCCTAAGCTGCTGATCTACGAT |
| | | GCATCCAATTTGGAAACAGGGGTCCCATCAAGGTTCAGTGGAAGTGGATC |
| | | TGGGACAGATTTTACTTTCACCATCAGCAGCCTGCAGCCTGAAGATATTG |
| | | CAACATATTACTGTCTACAACTTTATGATTACCCCGTCACTTTCGGCGGA |
| | | GGGACCAAGGTGGAGATCAAACGAACTGTGGCTGCACCATCTGTCTTCAT |
| | | CTTCCCGCCATCTGATGAGCAGTTGAAATGTGGAACTGCCTCTGTTGTGT |
| | | GCCTGCTGAATAACTTCTATCCCAGAGAGGCCAAAGTACAGTGGAAGGTG |
| | | GATAACGCCCTCCAATCGGGTAACTCCCAGGAGAGTGTCACAGAGCAGGA |
| | | CAGCAAGGACAGCACCTACAGCCTCAGCAGCACCCTGACGCTGAGCAAAG |
| | | CAGACTACGAGAAACACAAAGTCTACGCCTGCGAAGTCACCCATCAGGGC |
| | | CTGAGCTCGGGGGTCACAAAGAGCTTCAACAGGGGAGAGTGT |
| 342 | Anti CD122 P2D12 Fab | CACGTGCAGCTGGTGGAGACTGGGGGAGGCTTGGTGCAGCCTGGGGGGTC |
| | HC (VH, joint CH1) | CCTGAGACTCTCCTGTGCAGCCTCTGGATTCACCTTTAGCAGCTATGCCA |
| | | TGAGCTGGGTCCGCCAGGCTCCAGGGAAGGGGCTGGAGTGGGTCTCAGCT |
| | | ATTAGTGGTAGTGGTGGTAGCACATACTACGCAGACTCCGTGAAGGGCCG |
| | | GTTCACCATCTCCAGAGACAATTCCAAGAACACGCTGTATCTGCAAATGA |
| | | ACAGCCTGAGAGCCGAGGACACGGCCGTATATTACTGTGCGAGAGATCTC |
| | | GGGGATTATTGGGGCCAGGGAACCCTGGTCACCGTCTCAAGCGCCTCCAC |
| | | CAAGGGCCCATCGGTCTTCCCCCTGGCACCCTCCTCCCAAGAGCACCTCT |
| | | GGGGGCACAGCGGCCCTGGGCTGCCTGGTCAAGGACTACTTCCCCGAACC |
| | | GGTGACGGTGTCGTGGAACTCAGGCGCCCTGACCAGCGGCGTCCACACCT |
| | | TCCCGGCTGTCCTACAGTCCTCAGGACTCTACTCCCTCAGCAGCGTAGTG |
| | | ACCGTGCCCTCCAGCAGCTTGGGCACCCAGACCTACATCTGCAACGTGAA |
| | | TCACAAGCCCAGCAACACCAAGGTGGACAAGAAAGTTGAGCCCAAATCTT |
| | | GT |
| 343 | Anti CD122 P2D12 scFv | CAGGTCCAGCTGCAGGAGTCCGGGCCAGGGCTGGTGAAACCAAGCGAAAC |
| | and Fc with knob | ACTGAGTCTGACATGTACCGTGAGTGGGGGGTCCATTAACAATAGTAACT |
| | modification | ACTATTGGTCATGGATCAGACAGAGCCCTGGAAGAGGCCTGGAGTGGATC |
| | | GGGGGATCTACTTCAGCGGCACCACATACTATAACCCATCACTGCAGAGC |
| | | CGGGTGACTATCTCCATTGACACCTCTAAGAATCAGTTCAGCCTGAAGCT |
| | | GAGCAGCGTGACCGCCGCTGATACAGCCATCTACTATTGCGTCCGGCAGA |
| | | TGAATTACTATCACCTGGGCTCTAGTGTGGGGTTCGACCCCTGGGGACAG |
| | | GGAGCACTGGCCACCGTGTCAAGCGTGTCCTCTGGAGGAGGAGGCAGCGG |
| | | CGGAGGAGGCTCTGGAGGAGGGGGGAGTGATGTGGTCATGACACAGAGCC |
| | | CAGCTACTCTGTGTGTGAGTCCCGGCGAAAGGGCCACACTGAGCTGTCGC |
| | | GCTTCACAGAGCGTCAGTTCAAACCTGGCATGGTACCAGCAGAAGCCAGG |
| | | ACAGGCACCTTCCCTGCTGATCTATGAGGCTTCTACACGAGCAACTGGCA |
| | | TTCCTGCTAGATTCTCCGGCTCTGGGAGTGGAACCGACTTTACTCTGACC |
| | | ATCAGCTCCCTGCAGAGCGAAGATTTTGCAATCTACTATTGTCAGCAGTA |
| | | TAACGATTGGCTGTGGACCTTCGGGGAGGGGACTAAAGTGGAGATTCGGA |
| | | ACAGCGGGGGGGGCACCGGGGGGGGACTCACACATGCCCACCGTGCCCAG |
| | | CACCTGAAGCCGGGGGGGGACCGTCAGTCTTCCTCTTCCCCCCAAAACCC |
| | | AAGGACACCCTCATGATCTCCCGGACCCCTGAGGTCACATGCGTGGTGGT |
| | | GGACGTGAGCCACGAAGACCCTGAGGTCAAGTTCAACTGGTACGTGGACG |
| | | GCGTGGAGGTGCATAATGCCAAGACAAAGCCGCGGGAGGAGCAGTACAAC |
| | | AGCACGTACCGTGTGGTCAGCGTCCTCACCGTCCTGCACCAGGACTGGCT |
| | | GAATGGCAAGGAGTACAAGTGCAAGGTCTCCAACAAAGCCCTCCCAGCCC |
| | | CCATCGAGAAAACCATCTCCAAAGCCAAAGGGCAGCCCCGAGAACCACAG |
| | | GTGTACACCCTGCCCCCATGCCGGGATGAGCTGACCAAGAACCAGGTCAG |
| | | CCTGTGGTGCCTGGTCAAAGGCTTCTATCCCAGCGACATCGCCGTGGAGT |
| | | GGGAGAGCAATGGGCAGCCGGAGAACAACTACAAGACCACGCCTCCCGTG |
| | | CTGGACTCCGACGGCTCCTTCTTCCTCTACAGCAAGCTCACCGTGGACAA |
| | | GAGCAGGTGGCAGCAGGGGAACGTCTTCTCATGCTCCGTGATGCATGAGG |
| | | CTCTGCACAACCACTACACGCAGAAGAGCCTCTCCGTGTCTCCGGGTAAA |
| 344 | P1G11 Fab LC | AATTTTATGCTGACTCAGCCCCACTCTGTGTCGGAGTCTCCGGGGAAGAC |
| | (VL, joint CL) | GGTAACCATCTCCTGCACCCGCAGCAGTGGCAGCATTGCCAGCAACTATG |
| | | TGCAGTGGTACCAGCAGCGCCCGGGCAGTTCCCCCACCACGGTCATTTTT |
| | | GACGACAATCAAAGACCCACTGGTGTCCCTGATCGCTTCTCTGCCGCCAT |
| | | CGACACCTCCTCCAGTTCTGCCTCCCTCACCATCTCTGGACTGACGGCTG |
| | | AGGACGAGGCCGATTACTATTGTCAGTCGTCTCATAGCACCGCTGTCGTC |
| | | TTTGGCGGAGGGACCAAGCTGACCGTCCTAAGTCAGCCCAAGGCTGCCCC |
| | | CTCGGTCACTCTGTTCCCGCCCTCCTCTGAGGAGCTTCAAGCCAACAAGG |
| | | CCACACTGGTGTGTCTCATAAGTGACTTCTACCCGGGAGCCGTGACAGTG |
| | | GCCTGGAAGGCAGATAGCAGCCCCGTCAAGGGGGGAGTGGAGACCACCAC |
| | | ACCCTCCAAACAAAGCAACAACAAGTACGCGGCCAGCAGCTACCTGAGCC |
| | | TGACGCCTGAGCAGTGGAAGTCCCACAAAAGCTACAGCTGCCAGGTCACG |
| | | CATGAAGGGAGCACCGTGGAGAAGACAGTGGCCCCTACAGAATGTTCA |
| 345 | Anti CD122 P1G11 | CAGGTGCAGCTACAGCAGTGGGGCGCAGGACTGTTGAAGCCTTCGGAGAC |
| | Fab HC (VH, joint CH1) | CCTGTCCCTCACCTGCGCTGTCTATGGTGGGTCCTTCAGTGGTTACTACT |
| | | GGAGCTGGATCCGCCAGCCCCCAGGGAAGGGGCTGGAGTGGATTGGGGAA |
| | | ATCAATCATAGTGGAAGCACCAACTACAACCCGTCCCTCAAGAGTCGAGT |
| | | CACCATATCAGTAGACACGTCCAAGAACCAGTTCTCCCTGAAGCTGAGCT |
| | | CTGTGACCGCCGCGGACACGGCTGTGTATTACTGTGCGAGAAGCTCGTCC |
| | | GGGGATGCTTTTGATATCTGGGGCCAAGGGACAATGGTCACCGTCTCAAG |
| | | CGCCTCCACCAAGGGCCCATCGGTCTTCCCCCTGGCACCCTCCTCCAAGA |
| | | GCACCTCTGGGGGCACAGCGGCCCTGGGCTGCCTGGTCAAGGACTACTTC |
| | | CCCGAACCGGTGACGGTGTCGTGGAACTCAGGCGCCCTGACCAGGGGCGT |
| | | CCACACCTTCCCGGCTGTCCTACAGTCCTCAGGACTCTACTCCCTCAGCA |
| | | GCGTAGTGACCGTGCCCTCCAGCAGCTTGGGCACCCAGACCTACATCTGC |
| | | AACGTGAATCACAAGCCCAGCAACACCAAGGTGGAGAAGAAAGTTGAGGC |
| | | CAAATCTTGT |
| 346 | Anti CD122 P1G11 scFv | CAGGTGCAGCTACAGCAGTGGGGCGCAGGACTGTTGAAGCCTTCGGAGAC |
| | and Fc with knob | CCTGTCCCTCACCTGCGCTGTCTATGGTGGGTCCTTCAGTGGTTACTACT |
| | modification | GGAGCTGGATCCGCCAGCCCCCAGGGAAGGGGCTGGAGTGGATTGGGGAA |
| | | ATCAATCATAGTGGAAGCACCAACTACAACCCGTCCCTCAAGAGTCGAGT |
| | | CACCATATCAGTAGACACGTCCAAGAACCAGTTCTCCCTGAAGCTGAGCT |
| | | CTGTGACCGCCGCGGACACGGCTGTGTATTACTGTGCGAGAAGCTCGTCC |
| | | GGGGATGCTTTTGATATCTGGGGCCAAGGGACAATGGTCACCGTCTCAAG |
| | | CGGAGGAGGAGGATGTGGCGGAGGAGGCAGTGGAGGAGGAGGGTCACTTA |
| | | ATTTTATGCTGACTCAGCCCCACTCTGTGTCGGAGTCTCCGGGGAAGACG |
| | | GTAACCATCTCCTGCACCCGCAGCAGTGGCAGCATTGCCAGCAACTATGT |
| | | GCAGTGGTACCAGCAGCGCCCGGGCAGTTCCCCCACCACGGTCATTTTTG |
| | | ACGACAATCAAAGACCCACTGGTGTCCCTGATCGCTTCTCTGCCGCCATC |
| | | GACACCTCCTCCAGTTCTGCCTCCCTCACCATCTCTGGACTGACGGCTGA |
| | | GGACGAGGCCGATTACTATTGTCAGTCGTCTCATAGCACCGCTGTCGTCT |
| | | TTGGCGGAGGGACCAAGCTGACCGTCCTAAACAGCGGGGGGGGCACCGCG |
| | | GCCGCGACTCACACATGCCCACCGTGCCCAGCACCTGAAGCCGGGGGGGG |
| | | ACCGTCAGTCTTCCTCTTCCCCCCAAAACCCAAGGACACCCTCATGATCT |
| | | CCCGGACCCCTGAGGTCACATGCGTGGTGGTGGACGTGAGCCACGAAGAC |
| | | CCTGAGGTCAAGTTCAACTGGTACGTGGACGGCGTGGAGGTGCATAATGC |
| | | CAAGACAAAGCCGCGGGAGGAGCAGTACAACAGCACGTACCGTGTGGTCA |
| | | GCGTCCTCACCGTCCTGCACCAGGACTGGCTGAATGGCAAGGAGTACAAG |
| | | TGCAAGGTCTCCAACAAAGCCCTCCCAGCCCCCATCGAGAAAACCATCTC |
| | | CAAAGCCACAGGGCAGCCCCGAGAACCACAGGTGTACACCCTGCCCCCAT |
| | | GCCGGGATGAGCTGACCAAGAACCAGGTCAGCCTGTGGTGCCTGGTCAAA |
| | | GGCTTCTATCCCAGCGACATCGCCGTGGAGTGGGAGAGCAATGGGCAGCC |
| | | GGAGAACAACTACAAGACCACGCCTCCCGTGCTGGACTCCGACGGCTCCT |
| | | TCTTCCTCTACAGCAAGCTCACCGTGGACAAGAGCAGGTGGCAGCAGGGG |
| | | AACGTCTTCTCATGCTCCGTGATGCATGAGGCTCTGCACAACCACTACAC |
| | | GCAGAAGAGCCTCTCCGTGTCTCCGGGTAAA |
| 347 | Anti CD122 P1E7 | GATGTTGTGATGACTCAGTCTCCACTCTCCGTGCCCGTCACCCCTGGAGA |
| | Fab LC (VL, joint CL) | GCCGGCCTCCATCTCCTGCAGGTGTAGTCAGAGCCTCCTGCATAGTAATG |
| | | GATACAACTATTTGGATTGGTACCTGCAGAAGCCAGGGCAGTCTCCACAG |
| | | CTCCTGATCTATTTGGGTTCTAGTCGGGCCTCCGGGGTCCCTGACAGGTT |
| | | CAGTGGCAGTGGATCAGGCACAGATTTTACACTGAAAATCAGCAGAGTGG |
| | | AGGCTGAGGATGTTGGGGTTTATTACTGCATGCAAGCTCTACAAACTCCT |
| | | CGCACTTTTGGGCAGGGGACCAAGCTGGAGATCAAACGAACTGTGGCTGC |
| | | ACCATCTGTCTTCATCTTCCCGCCATCTGATGAGCAGTTGAAATCTGGAA |
| | | CTGCCTCTGTTGTGTGCCTGCTGAATAACTTCTATCCCAGAGAGGCCAAA |
| | | GTACAGTGGAAGGTGGATAACGCCCTCCAATCGGGTAACTCCCAGGAGAG |
| | | TGTCACAGAGCAGGACAGCAAGGACAGCACCTACAGCCTCAGCAGCACCC |
| | | TGACGCTGAGCAAAGCAGACTACGAGAAACACAAAGTCTACGCCTGCGAA |
| | | GTCACCCATCAGGGCCTGAGCTCGCCCGTCACAAAGAGCTTCAACAGGGG |
| | | AGAGTGT |
| 348 | Anti CD122 P1E7 | GAGGTGCAGCTGGTGCAGTCTGGGGGAGGCGTGGTCCAGCCTGGGAGGTC |
| | Fab HC (VH, joint CH1) | CCTGAGACTCTCCTGTGCAGCCTCTGGATTCACCTTCAGTAGCTATGCTA |
| | | TGCACTGGGTCCGCCAGGCTCCAGGCAAGGGGCTGGAGTGGGGGGAGTCA |
| | | TATCATATGATGGAAGCAATAAATACTATGCAGACTCCGTGAAGGGCCGA |
| | | TTCACCATCTCCAGAGACAATTCCAAGAACACGCTGTATCTGCAAATGAA |
| | | CAGCCTGAGAGCTGAGGACACGGCTGTGTATTACTGTGCGAGAGATCTCG |
| | | GGTATAGCAGCAGCTGGTACTACTACTACTACGGTATGGACGTCTGGGGC |
| | | CAAGGGACCACGGTCACCGTCTCAAGCGCCTCCACCAAGGGCCCATCGGT |
| | | CTTCCCCCTGGCACCCTCCTCCAAGAGCACCTCTGGGGGCACAGCGGCCC |
| | | TGGGCTGCCTGGTCAAGGACTACTTCCCCGAACCGGTGACGGTGTCGTGG |
| | | AACTCAGGCGCCCTGACCAGCGGCGTCCACACCTTCCCGGCTGTCCTAGA |
| | | GTCCTCAGGACTCTACTCCCTCAGCAGCGTAGTGACCGTGCCCTCCAGCA |
| | | GCTTGGGCACCCAGACCTACATCTGCAACGTGAATCACAAGCCCAGCAAC |
| | | ACCAAGGTGGACAAGAAAGTTGAGCCCAAATCTTGT |
| 349 | Anti CD122 P1E7 | GAGGTGCAGCTGGTGCAGAGCGGGGGGGGGGGGTGCAGCCTGGGAGGTCA |
| | scFv and Fc with | CTGAGACTGAGTTGTGCCGCATCCGGGTTTACATTTAGCTCCTATGCAAT |
| | knob modification | GCACTGGGTGAGGCAGGCCCCTGGCAAGGGGCTGGAGTGGGTGGCTGTCA |
| | | TCAGTTACGACGGCTCAAACAAGTACTATGCAGATTCTGTGAAAGGGGGG |
| | | TTCACAATTAGCAGAGACAACTCCAAAAATACTCTGTACCTCCAGATGAA |
| | | TAGCCTGCGAGCCGAAGACACCGCCGTGTACTATTGCGCCAGAGACCTGG |
| | | GATACTCTAGTTCATGGTACTACTACTACTACGGGATGGACGTGTGGGGA |
| | | CAGGGCACCACAGTGACAGTCAGCTCCGGCGGAGGAGGCTCAGGAGGAGG |
| | | AGGGTCCGGGGGAGGAGGATCTGATGTGGTCATGACCCAGTCCCCACTGT |
| | | CTCTGCCAGTGACACCTGGCGAGCCAGCAAGCATCAGCTGCCGGAGCAGC |
| | | CAGTCTCTGCTGCATAGTAACGGGTATAATTACCTGGACTGGTACTTGCA |
| | | GAAGCCTGGCCAGAGTCCTCAGCTGCTGATCTACCTGGGGTCAAGCAGGG |
| | | CCTCCGGAGTGCCCGACCGCTTCAGTGGGTCAGGAAGCGGCACTGACTTC |
| | | ACCCTGAAGATCAGCCGGGTGGAGGCTGAAGATGTGGGCGTCTATTACTG |
| | | TATGCAGGCACTGCAGACACCACGGACTTTTGGACAGGGGACTAAACTGG |
| | | AAATCAAGAACAGCGGGGGGGGCACCGCGGCCGCGACTCACACATGCCCA |
| | | CCGTGCCCAGCACCTGAAGCCGCGGGGGGACCGTCAGTCTTCCTCTTCCC |
| | | CCCAAAACCCAAGGACACCCTCATGATCTCCCGGACCCCTGAGGTCACAT |
| | | GCGTGGTGGTGGACGTGAGCCACGAAGACCCTGAGGTCAAGTTCAACTGG |
| | | TACGTGGACGGCGTGGAGGTGCATAATGCCAAGACAAAGCCGCGGGAGGA |
| | | GCAGTACAACAGCACGTACCGTGTGGTCAGCGTCCTCACCGTCCTGCACC |
| | | AGGACTGGCTGAATGGCAAGGAGTACAAGTGCAAGGTCTCCAACAAAGCC |
| | | CTCCCAGCCCCCATCGAGAAAACCATGTCCAAAGCCAAAGGGCAGCCCCG |
| | | AGAACCACAGGTGTACACCCTGCCCCCATGCCGGGATGAGCTGACCAAGA |
| | | ACCAGGTCAGCCTGTGGTGCCTGGTCAAAGGCTTCTATCCCAGCGACATC |
| | | GCCGTGGAGTGGGAGAGCAATGGGCAGCCGCTACAGCAAGCTCACCGTGG |
| | | ACAAGAGCAGGTGGCAGCAGGGGAACGTCTTCTCATGCTCCGTGATGCAT |
| | | GAGGCTCTGCACAACCACTACACGCAGAAGAGCCTCTCCCTGTCTCCGGG |
| | | TAAA |
| 350 | Anti CD122 P1B10 | GACATCCAGATGACCCAGTCTCCATCCTCCCTGTCTGCATCTGTAGGAGA |
| | Fab LC (VL, joint CL) | CAGAGTCACCATCACTTGCCAGGCGAGTCAGGACATTAGCGACTATTTAA |
| | | ATTGGTATCAGCAGAAACCAGGGAAAGCCCCTCAGATCCTGATCTACGAT |
| | | GCATCCAATTTGGAGACAGGGGTCCCATCAAGATTCAGTGGAAGTGGGTC |
| | | TGGGACAGATTTTACTTTCACCATCAGCAACCTGCAGCCTGAGGATGTTG |
| | | CAACATATTACTGTCAACAGTATGAGGATCTCCCCTCTTTCGGCGGAGGG |
| | | ACCAAGGTGGAGATCAAACGAACTGTGGCTGCACCATCTGTCTTCATCTT |
| | | CCCGCCATCTGATGAGCAGTTGAAATCTGGAACTGCCTCTGTTGTGTGCC |
| | | TGCTGAATAACTTCTATCCCAGAGAGGCCAAAGTACAGTGGAAGGTGGAT |
| | | AACGCCCTCCAATCGGGTAACTCCCAGGAGAGTGTCACAGAGCAGGACAG |
| | | CAAGGACAGCACCTACAGCCTCAGCAGCACCCTGACGCTGAGCAAAGCAG |
| | | ACTACGAGAAACACAAAGTCTACGCCTGCGAAGTCACCCATCAGGGCCTG |
| | | AGCTCGCCGGTCACAAAGAGCTTCAACAGGGGAGAGTGT |
| 351 | Anti CD122 P1B10 Fab HC | CAGGTGCAGCTGCAGGAGTCGGGCCCAGGACTGGTGAAGCCTTCGGAGAC |
| | (VH, joint CH1) | CCTGTCCCTCACCTGCACTGTCTCTGGTGTCTCCATCAGCAGTAGAAGTG |
| | | ACCACTGGGGGTGGGTCCGCCAGCCCCCAGGGAAGGGGCTGGAGTGGATT |
| | | GGAAGTATCTCTTATAGTGGGAGCACCTACTACAACCCGTCCCTCAAGAG |
| | | CCGAGTCACCATATCCGTAGACACCTCCAAGAACCAACTCTCCCTGAAGC |
| | | TGAGCTCTTGTGACCGCCGCAGACACGGCTGTGTATTACTGTGCGAGAGA |
| | | GTCGCACCCAGCAGGTGCACTGGTTGGGTGGGGCCAGGGCACCCTGGTCA |
| | | CCGTCTCAAGCGCCTCCACCAAGGGCCCATCGGTCTTCCCCCTGGCACCC |
| | | TCCTCCAAGAGCACCTCTGGGGGCACAGCGGCCCTGGGCTGCCTGGTCAA |
| | | GGACTACTTCCCCGAACCGGTGACGGTGTCGTGGAACTCAGGCGCCCTGA |
| | | CCAGCGGCGTCCACACCTTCCCGGCTGTCCTACAGTCCTCAGGACTCTAC |
| | | TCCCTCAGCAGCGTAGTGACCGTGCCCTCCAGCAGCTTGGGCACCCAGAC |
| | | CTACATCTGCAACGTGAATCACAAGCCCAGCAACACCAAGGTGGACAAGA |
| | | AAGTTGAGCCCAAATCTTGT |
| 352 | Anti | CAGGTCCAGCTGCAGGAGAGGGGCCCCGGACTGGTGAAGCCTAGCGAAAC |
| | CD122 P1B10 scFv and | ACTGAGCCTGACTTGTACTGTGAGCGGCGTGAGCATTAGCTCCCGGAGCG |
| | Fc with knob | ACCACTGGGGATGGGTGAGACAGCCCCCTGGCAAGGGGCTGGAGTGGATC |
| | modification | GGAAGTATTTCATACAGCGGCTCCACTTACTATAACCCCTCTCTGAAAAG |
| | | TAGGGTGACTATGTCAGTGGACACCAGCAAGAATCAGCTGAGTGTGAAAC |
| | | TGTCTAGTGTGACCGCCGCTGATACAGGAGTCTACTATTGCGCCCGCGAA |
| | | TCCCATCCTGCCGCCGCCCTGGTGGGATGGGGACAGGGGACACTGGTGAC |
| | | TGTCTCAAGCGGAGGAGGAGGCAGTGGAGGAGGAGGGTCAGGAGGCGGGG |
| | | GAAGCGACATTCAGATGACACAGAGCCCATCCTCTCTGTCTGCCAGTGTG |
| | | GGCGATCGAGTCACCATCACATGTCAGGCTTCCCAGGACATTTCTGATTA |
| | | CCTGAACTGGTATCAGCAGAAGCCAGGGAAAGCTCCCCAGATCCTGATCT |
| | | ACGACGCATCCAATCTGGAGACAGGCGTGCCCAGCCGGTTCAGCGGAAGC |
| | | GGCTCGGGGACTGATTTCACTTTTACCATCTCTAACCTCCAGCCTGAGGA |
| | | CGTGGCCACCTACTATTGCCAGCAGTATGAGGACCTGCCATCCTTTGGGG |
| | | GGGGAACAAAGGTCGAGATCAAGAACAGCGGGGGGGGCACCGCGGCCGCG |
| | | ACTCACACATGCCCACCGTGCCCAGCACCTGAAGCCGCGGGGGGACCGTC |
| | | AGTCTTCCTCTTCCCCCCAAAACCCAAGGACACCCTCATGATCTCCCGGA |
| | | CCCCTGAGGTCACATGCGTGGTGGTGGACGTGAGCCACGAAGACCCTGAG |
| | | GTCAAGTTCAACTGGTACGTGGACGGCGTGGAGGTGCATAATGCCAAGAC |
| | | AAAGCCGCGGGAGGAGCAGTACAACAGCACGTACCGTGTGGTCAGCGTCC |
| | | TCACCGTCCTGCACCAGGACTGGCTGAATGGCAAGGAGTACAAGTGCAAG |
| | | GTCTCCAACAAAGCCCTCCCAGCCCCCATCGAGAAAACCATCTCCAAAGC |
| | | CAAAGGGCAGCCCCGAGAACCACAGGTGTACACCCTGCCCCCATGCCGGG |
| | | ATGAGCTGACCAAGAACCAGGTCAGCCTGTGGTGCCTGGTCAAAGGCTTC |
| | | TATCCCAGCGACATCGCCGTGGAGTGGGAGAGCAATGGGCAGCCGGAGAA |
| | | CAACTACAAGACCACGCCTCCCGTGCTGGACTCCGACGGCTCCTTCTTCC |
| | | TCTACAGCAAGCTCACCGTGGACAAGAGCAGGTGGCAGCAGGGGAACGTC |
| | | TTCTCATGCTCCGTGATGCATGAGGCTCTGCACAACCACTACACGCAGAA |
| | | GAGCCTCTCCCTGTCTCCGGGTAAA |
| 353 | Anti CD122 P1F3 Fab | GACATCCAGTTGACCCAGTCTCCATCCTCCCTGTCTGCATCTGTAGGAGA |
| | LC (VL, joint CL) | CAGAGTCACCATCACTTGCCGGGCAAGTCAGAGCATTAGCAGCTATTTAA |
| | | ATTGGTATCAGCAGAAACCAGGGAAAGCCCCTAAGGCCCTGATCTACGAT |
| | | GCATCCAATTTGGAAACAGGGGTCCCATCAAGGTTCAGTGGCAGTGGATC |
| | | TGGGACAGATTTCACTCTCACCATCATCAGTCTGCAACCTGAAGATTTTG |
| | | CAACTTATTTCTGTCTACAAGATTACATTTACCCGTGGACGTTCGGCCAA |
| | | GGGACCAAGGTGGAATTCAAACGAACTGTGGCTGCACCATCTGTCTTCAT |
| | | CTTCCCGCCATCTGATGAGCAGTTGAAATCTGGAACTGCCTCTGTTGTGT |
| | | GCCTGCTGAATAACTTCTATCCCAGAGAGGCCAAAGTACAGTGGAAGGTG |
| | | GATAACGCCCTCCAATCGGGTAACTCCCAGGAGAGTGTCACAGAGCAGGA |
| | | CAGCAAGGACAGCACCTACAGCCTCAGCAGCACCCTGACGCTGAGCAAAG |
| | | CAGACTACGAGAAACACAAAGTCTACGCCTGCGAAGTCACCCATCAGGGC |
| | | CTGAGCTCGCCCGTCACAAAGAGCTTCAACAGGGGAGAGTGT |
| 354 | Anti CD122 P1F3 Fab | GAGGTGCAGCTGGTGCAGTCTGGGGGAGGCTTGGTACAGCCTGGGGGGTC |
| | HC (VH, joint CH1) | CCTGAGACTCTCCTGTGCAGCCTCTGGATTCACCTTTAGCAGCTATGCCA |
| | | TGAGCTGGGTCCGCCAGGCTCCAGGGAAGGGGCTGGAGTGGGTCTCAGCT |
| | | ATTAGTGGTAGTGGTGGCAGCACACACTACGCAGACTCCGTGAAGGGCCG |
| | | GTTCACCATCTCCAGAGACAACTCCAAGAACACGCTGTATCTGCAAATGA |
| | | ACAGCCTGAGAGCCGAGGACACGGCCGTATATTACTGTGCGACTCCGGCT |
| | | TTCTGGGGCCAGGGAACCCTGGTCACCGTCTCAAGCGCCTCCACCAAGGG |
| | | CCCATCGGTCTTCCCCCTGGCACCCTCCTCCAAGAGCACCTCTGGGGGCA |
| | | CAGCGGCCCTGGGCTGCCTGGTCAAGGACTACTTCCCCGAACCGGTGACG |
| | | GTGTCGTGGAACTCAGGCGCCCTGACCAGCGGCGTCCACACCTTCCCGGC |
| | | TGTCCTACAGTCCTCAGGACTCTACTCCCTCAGCAGCGTAGTGACCGTGC |
| | | CCTCCAGCAGCTTGGGCACCCAGACCTACATCTGCAACGTGAATCACAAG |
| | | CCCAGCAACACCAAGGTGGACAAGAAAGTTGAGCCCAAATCTTGT |
| 355 | Anti CD122 P1F3 | GAGGTGCAGCTGGTGCAGAGGGGGGGAGGACTGGTGCAGCCTGGGGGGTC |
| | scFv and Fc with | ACTGAGACTGAGTTGTGCCGCAAGCGGGTTTACATTTAGCTCCTACGCCA |
| | knob modification | TGTCTTGGGTGCGACAGGCTCCCGGAAAAGGCCTGGAGTGGGTCAGCGCA |
| | | ATCAGTGGATCAGGCGGGTCTACTCACTACGCCGACAGTGTGAAAGGCCG |
| | | GTTCACCATCAGCCGGGACAAGAGTAAGAATACTCTGTACCTCCAGATGA |
| | | ACAGCCTGAGAGCTGAAGACACCGCCGTGTACTATTGCGCCACCCCTGCT |
| | | TTTTGGGGGCAGGGAACACTGGTGACTGTCTCTAGTGGAGGAGGAGGATC |
| | | AGGGGGGGGAGGCAGCGGAGGAGGAGGGTCCGACATCCAGCTGACACAGT |
| | | CCCCATCAAGCCTGAGCGCTTCCGTGGGCGATAGGGTCACCATCACATGT |
| | | CGCGCATCTCAGAGTATTTCCTCTTACCTGAACTGGTATCAGCAGAAGCC |
| | | CGGCAAGGCACCTAAGGCCCTGATCTACGACGCCAGCAATCTGGAGACCG |
| | | GCGTGCCTTCCCGGTTCTCAGGCAGCGGGTCCGGAACAGATTTTACTCTG |
| | | ACCATCATCAGCCTCCAGCCAGAGGACTTCGCTACCTATTTTTGCCTCCA |
| | | GGATTACATCTACCCCTGGACCTTCGGCCAGGGGACAAAAGTGGAGTTCA |
| | | AGAACAGCGGGGGGGGCACCGGGGGCGCGACTCACACATGCCCACCGTGC |
| | | CCAGCACCTGAAGCCGGGGGGGGACCGTCAGTCTTCCTCTTCCCCCCAAA |
| | | ACCCAAGGACACCCTCATGATCTCCCGGACCCCTGAGGTCACATGCGTGG |
| | | TGGTGGACGTGAGCCACGAAGACCCTGAGGTCAAGTTCAACTGGTACGTG |
| | | GACGGCGTGGAGGTGCATAATGCCAAGACAAAGCCGCGGGAGGAGCAGTA |
| | | CAACAGCACGTACCGTGTGGTCAGCGTCGTCACCGTCCTGCACCAGGACT |
| | | GGCTGAATGGCAAGGAGTACAAGTGCAAGGTCTCCAACAAAGCCCTCCCA |
| | | GCCCCCATCGAGAAAACCATCTCCAAAGCCAAAGGGCAGCCCCGAGAACC |
| | | ACAGGTGTACACCCTGCCCCCATGCCGGGATGAGCTGACCAAGAACCAGG |
| | | TCAGCCTGTGGTGCCTGGTCAAAGGCTTCTATCCCAGCGACATCGCCGTG |
| | | GAGTGGGAGAGCAATGGGCAGCCGGAGAACAACTACAAGACCACGCCTCC |
| | | CGTGCTGGACTCCGACGGCTCCTTCTTCCTCTACAGCAAGCTCACCGTGG |
| | | ACAAGAGCAGGTGGCAGCAGGGGAACGTCTTCTCATGCTCCGTGATGCAT |
| | | GAGGCTCTGCACAACCACTACACGCAGAAGAGCCTCTCCCTGTCTCCGGG |
| | | TAAA |
| 356 | Anti CD122 P1D10 | CAGTCTGTGTTGACGCAGCCGCCCTCAGTGTGTGGGGCCCCAGGGCAGAG |
| | Fab LC (VL, joint CL) | GGTCACCATCTCCTGCACTGGGGGCAGCTCCAACGTCGGGGCAGGTTATG |
| | | ATGTACACTGGTACCAGCAGCTTCCAGGAACAGTCCCCAAACTCCTCATC |
| | | TATGATAACACCAATCGGCCCTCAGGTGTCCCTGACCGGTTCTCTGCCTC |
| | | CAAGTGTGGCACCTCAGCCTCTCTGGTCATCACTGGGCTCCAGGCTGAGG |
| | | ATGAGGGTGACTATTACTGCCAGTCGTATGACAGTAGTCTGCGTGCTTCG |
| | | GTATTCGGCGGAGGGACCATGTTGACCGTCCTAAGTCAGCCCAAGGCTGC |
| | | CCCCTCGGTCACTCTGTTCCCGCCCTCCTCTGAGGAGCTTCAAGCCAACA |
| | | AGGCCACACTGGTGTGTCTCATAAGTGACTTCTACCCGGGAGCCGTGACA |
| | | GTGGCCTGGAAGGCAGATAGCAGCCCCGTCAAGGGGGGAGTGGAGACCAC |
| | | CACACCCTCCAAACAAAGCAACAACAAGTACGCGGCCAGCAGCTATCTGA |
| | | GCCTGACGCCTGAGCAGTGGAAGTCCCACAAAAGCTACAGCTGCCAGGTC |
| | | ACGCATGAAGGGAGCACCGTGGAGAAGACAGTGGCCCCTAGAGAATGTTC |
| | | A |
| 357 | Anti CD122 P1D10 | CAGGTGCAGCTACAGCAGTGGGGCCCAGGACTGGTGAAGCCTTCGGAGAC |
| | Fab HC (VH, joint CH1) | CCTGTCCCTCACCTGCACTGTCTCTGGTGGCTCCATCAGTAGTTACTACT |
| | | GGAGCTGGATCCGCCAGCCCCCAGGGAAGGGGCTGGAGTGGATTGGGGAA |
| | | ATCAATCATAGTGGAAGCACCAACTACAACCCGTCCCTCAAGAGTCGAGT |
| | | CACCATATCAGTAGACACGTCCAAGAACCAGTTCTCCCTGAAGCTGAGCT |
| | | CTGTGACCGCCGCGGACACGGCTGTGTATTACTGTGCGGGAGGCTCTAAT |
| | | TTGGACTGGTTCGACCCCTGGGGCCAGGGAACCCTGGTCACCGTCTCAAG |
| | | CGCCTCCACCAAGGGCCCATCGGTCTTCCCCCTGGCACCCTCCTCCAAGA |
| | | GCACCTCTGGGGGCACAGCGGCCCTGGGCTGCCTGGTCAAGGACTACTTC |
| | | CCCGAACCGGTGACGGTGTCGTGGAACTCAGGCGCCCTGACCAGCGGCGT |
| | | CCACACCTTCCCGGCTGTCCTACAGTCCTCAGGACTCTACTCCCTCAGCA |
| | | GCGTAGTGACCGTGCCCTCCAGCAGCTTGGGCACCCAGACCTACATCTGC |
| | | AACGTGAATCACAAGCCCAGCAACACCAAGGTGGACAAGAAAGTTGAGCC |
| | | CAAATCTTGT |
| 358 | Anti CD122 P1D10 scFv | CAGGTCCAGCTGCAGCAGTGGGGGCCAGGACTGGTGAAGCCATCCGAAAC |
| | and Fc with | TCTGTCTCTGACTTGTACCGTGAGGGGGGGGAGCATCAGCTCCTACTATT |
| | knob modification | GGAGCTGGATCAGGCAGCCCCCTGGGAAGGGACTGGAGTGGATCGGCGAA |
| | | ATTAACCACAGCGGGTCCACTAACTACAATCCTTCCCTGAAATCTCGCGT |
| | | GACTATTAGTGTGGACACCTCAAAGAATCAGTTCTCCCTGAAACTGTCTA |
| | | GTGTGACAGCCGCTGATACCGCCGTGTACTATTGCGCCGGGGGGTCTAAC |
| | | CTGGACTGGTTTGATCCCTGGGGACAGGGGACCCTGGTGACAGTCTCAAG |
| | | CGGAGGAGGAGGAAGCGGCGGAGGAGGCTCCGGAGGAGGAGGGTCTCAGA |
| | | GTGTGCTGACACAGCCACCATCAGTCAGCGGGGCCCCCGGACAGCGAGTG |
| | | ACCATCTCCTGTACAGGAGGCTCCTCTAATGTGGGAGCCGGCTACGACGT |
| | | CCATTGGTATCAGCAGCTGCCTGGCACCGTGCCAAAGCTGGTGATCTACG |
| | | ACAACAGAAATCGGCCCAGCGGGGTGCCTGATAGATTCTCCGCTTCTAAA |
| | | AGTGGCACATCAGCCAGCCTGGTCATCACTGGACTCCAGGCCGAGGACGA |
| | | AGGGGATTACTATTGCCAGTCTTATGATAGTTCACTGAGAGCTAGTGTGT |
| | | TTGGGGGAGGCACTATGCTGACCGTCCTGAAGAGCGGGGGGGGCACCGGG |
| | | GCCGCGAGTCACACATGCCGACCGTGCCCAGCACCTGAAGCCGCGGGGGG |
| | | ACCGTCAGTCTTCCTCTTCCCCCCAAAACCCAAGGACACCCTCATGATCT |
| | | CCCGGACCCCTGAGGTCACATGCGTGGTGGTGGACGTGAGCCACGAAGAC |
| | | CCTGAGGTCAAGTTCAACTGGTACGTGGACGGCGTGGAGGTGCATAATGC |
| | | CAAGACAAAGCCGCGGGAGGAGCAGTACAACAGCACGTACCGTGTGGTCA |
| | | GCGTCCTCACCGTCCTGCACCAGGACTGGCTGAATGGCAAGGAGTACAAG |
| | | TGCAAGGTCTCCAACAAAGCCCTCCCAGCCCCCATCGAGAAAACCATCTC |
| | | CAAAGCCAAAGGGCAGCCCCGAGAACCACAGGTGTACACCCTGCCCCCAT |
| | | GCCGGGATGAGCTGACCAAGAACCAGGTCAGCCTGTGGTGCCTGGTCAAA |
| | | GGCTTCTATCCCAGCGACATCGCCGTGGAGTGGGAGAGCAATGGGCAGCC |
| | | GGAGAACAACTACAAGACCACGCCTCCCGTGCTGGACTCCGACGGCTCCT |
| | | TCTTCCTCTACAGCAAGCTCACCGTGGACAAGAGCAGGTGGCAGCAGGGG |
| | | AACGTCTTCTCATGCTCCGTGATGCATGAGGCTCTGCACAACCACTACAC |
| | | GCAGAAGAGCCTCTCCCTGTCTCCGGGTAAA |
| 359 | Anti CD122 P1E1 Fab | AATTTTATGCTGACTCAGCCCCACTCTGTGTCGGAGTCTGGGGGGAAGAC |
| | LC (VL, joint CL) | GGTAACCATCTCCTGCACCGGCAGCAGTGGCAGCATTGCCAGCAGCTATG |
| | | TGCAGTGGTACCAGCAGCGCCCGGGCAGTGCCCCCACCACTGTGATCTAT |
| | | GCGGATAACCAAAGACCCTCTGGGGTCCCTGATCGGTTCTCTGGCTCCGT |
| | | CGACAGCTCCTCCAACTCTGCCTCCCTCACCATCTCTGGACTGAAGACTG |
| | | AGGACGAGGCTGACTACTACTGTCAGTCTTTTGACAGCAGCCTCTATATG |
| | | ATTTTTGGGGGAGGGACCAAGCTGACCGTCCTAGGTCAGCCCAAGGCTGC |
| | | CCCCTCGGTCACTCTGTTCCCGCCCTCCTCTGAGGAGCTTCAAGCCAACA |
| | | AGGCCACACTGGTGTGTCTCATAAGTGACTTCTACCCGGGAGCCGTGACA |
| | | GTGGCCTGGAAGGCAGATAGCAGCCCCGTCAAGGCGGGAGTGGAGACCAC |
| | | CACACCCTCCAAACAAAGCAACAACAAGTACGCGGCCAGCAGCTATCTGA |
| | | GCCTGACGCCTGAGCAGTGGAAGTCCCACAGAAGCTACAGCTGCCAGGTC |
| | | ACGCATGAAGGGAGCACCGTGGAGAAGACAGTGGCCCCTGCAGAATGCTC |
| | | T |
| 360 | Anti CD122 P1E1 Fab | CAGGTGCAGCTACAGCAGTGGGGCGCAGGACTGTTGAAGCCTTCGGAGAC |
| | HC (VH, joint CH1) | CCTGTCCCTCACCTGCGCTGTCTATGGTGGGTCCTTCAGTGGTTACTACT |
| | | GGAGCTGGATCCGCCAGCCCCCAGGGAAGGGGCTGGAGTGGATTGGGGAA |
| | | ATCAATCATAGTGGAAGCACCAACTACAACCCGTCCCTCAAGAGTCGAGT |
| | | CACCATATCAGTAGACACGTCCAAGAACCAGTTCTCCCTGGAGCTGAGCT |
| | | CTGTGACCGCCGCGGACACGGCTGTGTATTACTGTGCGAGAGCGGATCGT |
| | | CGGTTCGGGGAGTTACGCTACTGGGGCCAGGGAACCCTGGTCACCGTCTC |
| | | AAGCGCCTCCACCAAGGGCCCATCGGTCTTCCCCCTGGCACCCTCCTCCA |
| | | AGAGCACCTCTGGGGGCACAGCGGCCCTGGGCTGCCTGGTCAAGGACTAC |
| | | TTCCCCGAACCGGTGACGGTGTCGTGGAACTCAGGCGCCCTGACCAGCGG |
| | | CGTCCACACCTTCCCGGCTGTCCTACAGTCCTCAGGACTCTACTCCCTCA |
| | | GCAGCGTAGTGACCGTGCCCTCCAGCAGCTTGGGCACCCAGACCTACATC |
| | | TGCAACGTGAATCACAAGCCCAGCAACACCAAGGGGACAAGAAAGTTGAG |
| | | CCCAAATCTTGT |
| 361 | Anti CD122 P1E1 scFv | CAGGTCCAGCTGCAGCAGTGGGGAGCCGGACTGCTGAAGCCAAGTGAGAC |
| | and Fc with knob | TCTGAGCCTGACATGCGCCGTGTATGGGGGAAGTTTTTCCGGCTACTATT |
| | modification | GGTCTTGGATCAGACAGCCCCCTGGCAAGGGGCTGGAGTGGATCGGCGAA |
| | | ATTAACCACAGTGGGTCAACCAACTACAATCCCTCTCTGAAGAGTCGCGT |
| | | GACAATTTCAGTGGACACTAGCAAAAATCAGTTCAGCCTGGAGCTGAGCA |
| | | GCGTGACTGCCGCTGACACCGCCGTCTACTATTGCGCACGAGCCGATCGG |
| | | AGATTTGGCGAACTGGGGTATTGGGGACAGGGCACACTGGTGACTGTCTC |
| | | TAGTGGAGGAGGAGGCAGTGGAGGAGGAGGGTCAGGAGGCGGGGGATCTA |
| | | ACTTCATGCTGACTCAGCCCCATAGCGTGTCCGAGTCTCCTGGGAAAACT |
| | | GTCACCATCAGTTGTACAGGGTCAAGCGGATCTATTGCCTCCTCTTACGT |
| | | GCAGTGGTATCAGCAGAGGCCAGGCTCCGCTCCCACCACAGTGATCTACG |
| | | CAGACAACCAGAGGCCTAGCGGAGTGCCAGACCGCTTTAGTGGCTCAGTC |
| | | GATAGTTCAAGCAATAGCGCCTCCCTGACCATCTCCGGCCTGAAGACAGA |
| | | GGACGAAGCTGATTACTATTGCCAGAGCTTCGATTCCTCTCTGTATATGA |
| | | TTTTTGGGGGGGGAACCAAACTGACAGTGCTGAACAGCGGGGGGGGCACC |
| | | GCGGCCGCGACTCACACATGCCCACCGTGCCCAGCACCTGAAGCCGCGGG |
| | | GGGACCGTCAGTCTTCCTCTTCCCCCCAAAACCCAAGGACACCCTCATGA |
| | | TCTCCCGGACCCCTGAGGTCACATGCGTGGTGGTGGACGTGAGCCACGAA |
| | | GACCCTGAGGTCAAGTTCAACTGGTACGTGGACGGCGTGGAGGTGCATAA |
| | | TGCCAAGACAAAGCCGCGGGAGGAGCAGTACAACAGCACGTACCGTGTGG |
| | | TCAGCGTCCTCACCGTCCTGCACCAGGACTGGCTGAATGGCAAGGAGTAC |
| | | AAGTGCAAGGTCTCCAACAAAGCCCTCCCAGCCCCCATCGAGAAAACCAT |
| | | CTCCAAAGCCAAAGGGCAGCCCCGAGAACCACAGGTGTACACCCTGCCCC |
| | | CATGCCGGGATGAGCTGACCAAGAACCAGGTCAGCCTGTGGTGCCTGGTC |
| | | AAAGGCTTCTATCCCAGCGACATCGCCGTGGAGTGGGAGAGCAATGGGCA |
| | | GCCGGAGAACAACTACAAGACCACGCCTCCCGTGCTGGACTCCGACGGCT |
| | | CCTTCTTCCTCTACAGCAAGCTCACCGTGGACAAGAGCAGGTGGCAGCAG |
| | | GGGAACGTCTTCTCATGCTCCGTGATGCATGAGGCTCTGCACAACCACTA |
| | | CACGAGAAGAGCCTCTCCCTGTCTCGGGGTAAA |
| 362 | Anti CD122 P2B11 | CAGTCTGTGTTGACGCAGCCGCCCTCAGTGTCTGGGGCCCCAGGGCAGAG |
| | Fab LC (VL, joint CL) | GGTCACCATCTCCTGCACTGGGAGCCGCTCCAACATCGGGGCAGGTTATG |
| | | ATGTACACTGGTATCAGCATCTTCCAGGGACAGCCCCCAAACTCCTCATC |
| | | TATGATAACAGCAATCGACCCTCAGGTGTCTCTGACCGATTCTCTGGCTC |
| | | CAAGTCTGGCACCTCAGCCTCGCTGGCCATCACTGGGCTCCAGGCTGAGG |
| | | ATGAGGCTGATTATTACTGCCAGTCCTTTGACAGCAGCCTGAGGGGTGTG |
| | | GTGTTCGGCGGAGGGACCAGGCTGACCGTCCTAAGTCAGCCCAAGGCTGC |
| | | CCCCTCGGTCACTCTGTTCCCGCCCTCCTCTGAGGAGCTCCAAGCCAACA |
| | | AGGCCACACTAGTGTGTCTGATCAGTGACTTCTACCCGGGAGCTGTGACA |
| | | GTGGCCTGGAAGGCAGATGGCCGCCCCGTCAAGGGGGGAGTGGAGACCAC |
| | | CACACCCTCCAAACAAAGCAACAACAAGTACGCGGCCAGCAGCTACCTGA |
| | | GCCTGACGCCTGAGCAGTGGAAGTCCCACAAAAGCTACAGCTGCCAGGTC |
| | | ACGCATGAAGGGAGCACCGTGGAGAAGACAGTGGCCCCTACAGAATGTTC |
| | | A |
| 363 | Anti CD122 P2B11 | CAGGTCCAGCTGGTGCAGTGTGGGGGAGGCCTGGTCAAGCCTGGGGGGTC |
| | Fab HC (VH, joint CH1) | CCTGAGACTCTCCTGTGCAGCCTCTGGATTCACCTTCAGTAGTTATGACT |
| | | TACACTGGGTCCGCCAGGTTCCAGGCAAGGGGCTGGAGTGGGTGTCACTT |
| | | ATATCATATGATGGAAGTAATAAATACTATGCAGACTCCGTGAAGGGCCG |
| | | ATTCACCATCTCCAGAGACAACGCCGAGAACTCACTGTATCTGCAAATGA |
| | | AGAGCCTGAGAGCTGAGGACACGGCTGTCTATTACTGTGCGAGAGAGCCT |
| | | ATAACTGGAACTTCTGACCTCTTTGACTACTGGGGCCAGGGAACCCTGGT |
| | | CACCGTCTCAAGCGCCTCCACCAAGGGCCCATCGGTCTTCCCCCTGGCAC |
| | | CCTCCTCCAAGAGCACCTCTGGGGGCACAGCGGCCCTGGGCTGCCTGGTC |
| | | AAGGACTACTTCCCCGAACCGGTGACGGTGTCGTGGAACTCAGGCGCCCT |
| | | GACCAGCGGCGTCCACACCTTCCCGGCTGTCCTACAGTCCTCAGGACTCT |
| | | ACTCCCTCAGCAGCGTAGTGACCGTGCCCTCCAGCAGCTTGGGCACCCAG |
| | | ACCTACATCTGCAACGTGAATCACAAGCCCAGCAACACCAAGGTGGACAA |
| | | GAAAGTTGAGCCCAAATCTTGT |
| 364 | Anti CD122 P2B11 | CAGGTGCAGCTGGTGCAGAGCGGGGGAGGACTGGTCAAGCCTGGAGGGTC |
| | scFv and Fc with | ACTGAGACTGTCATGTGCCGCAAGCGGATTCACTTTCAGCTCCTACGACC |
| | knob modification | TGCACTGGGTGAGGCAGGTCCCCGGCAAGGGGCTGGAGTGGGTGTCTCTG |
| | | ATCAGTTATGACGGGAGTAACAAGTACTATGCCGATTCAGTCAAAGGACG |
| | | GTTCACAATTTCCAGAGACAACGCTGAAAATTCTCTGTACCTCCAGATGA |
| | | ATAGTCTGCGCGCAGAGGATACTGCCGTGTACTATTGCGCCAGAGAGCCT |
| | | ATCACCGGCACAAGCGACCTGTTTGATTATTGGGGACAGGGCACTCTGGT |
| | | GACCGTCTCTAGTGGGGGAGGAGGCTCCGGAGGAGGAGGGTCTGGAGGAG |
| | | GAGGCAGCCAGTCTGTGCTGACCCAGCCACCTAGTGTCTCAGGCGCCCCT |
| | | GGGCAGCGAGTGACCATCTCCTGTACAGGCAGCCGGTCCAACATTGGGGC |
| | | AGGATACGACGTCCACTGGTATCAGCATCTGCCAGGCACAGCCCCCAAGC |
| | | TGCTGATCTACGACAACTCTAATAGGCCATCAGGGGTGAGCGATCGCTTC |
| | | TCTGGAAGTAAATCAGGCACTAGCGCCTCCCTGGCTATTACCGGCCTCCA |
| | | GGCTGAGGACGAAGCAGATTACTATTGCCAGTCCTTCGATTCAAGCCTGA |
| | | GAGGCGTGGTCTTTGGCGGGGGAACAAGGCTGACTGTGCTGAACAGCGGG |
| | | GGGGGCACCGCGGCCGCGACTCACACATGCCCACCGTGCCCAGCACCTGA |
| | | AGCCGGGGGGGGACCGTCAGTCTTCCTCTTCCCCCCAAAACCCAAGGACA |
| | | CCCTCATGATCTCCCGGACCCCTGAGGTCACATGCGTGGTGGTGGACGTG |
| | | AGCCACGAAGACCCTGAGGTCAAGTTCAACTGGTACGTGGACGGCGTGGA |
| | | GGTGCATAATGCCAAGACAAAGGGGGGGGAGGAGCAGTACAACAGCACGT |
| | | ACCGTGTGGTCAGCGTCCTCACCGTCCTGCACCAGGACTGGCTGAATGGC |
| | | AAGGAGTACAAGTGCAAGGTCTCCAACAAAGCCCTCCCAGCCCCCATCGA |
| | | GAAAACCATCTCCAAAGCCAAAGGGCAGCCCCGAGAACCACAGGTGTACA |
| | | CCCTGCCCCCATGCCGGGATGAGCTGACCAAGAACCAGGTCAGCCTGTGG |
| | | TGCCTGGTCAAAGGCTTCTATCCCAGCGACATCGCCGTGGAGTGGGAGAG |
| | | CAATGGGCAGCCGGAGAACAACTACAAGACCACGCCTCCCGTGCTGGACT |
| | | CCGACGGCTCCTTCTTCCTCTACAGCAAGCTCACCGTGGACAAGAGCAGG |
| | | TGGCAGCAGGGGAACGTCTTCTCATGCTCCGTGATGCATGAGGCTCTGCA |
| | | CAACCACTACACGCAGAAGAGCCTCTCCCTGTCTCCGGGTAAA |
| 365 | Anti CD122 P2C9 Fab | TCCTATGAGCTGACTCAGCCACCCTCAGCGTCTGGGACCCCCGGGCAGAG |
| | LC (VL joint CL) | GGTCACCATCTCTTGTTCTGGAAGCAGCTCCAACATCGGAAGTAATACTG |
| | | TAAACTGGTACCAGCAGCTCCCAGGAAGGGCCCCCAAACTCCTCATCTAT |
| | | AGTAATAATCAGCGGCCCTCAGGGGTCCCTGACCGATTCTCTGGCTCCAA |
| | | GTCTGGCACCTCAGCCTCCCTGGCCATCAGTGGGCTCCAGTCTGAGGATG |
| | | AGGCTGATTATTACTGTGCAGCATGGGATGACAGCCTGAATGGTCTTTGG |
| | | GTGTTCGGCGGAGGGACCAAGCTGACCGTCCTAGGTCAGCCCAAGGCTGC |
| | | CCCCTCGGTCACTCTGTTCCCACCCTCCTCTGAGGAGCTTCAAGCCAACA |
| | | AGGCCACACTGGTGTGTCTCATAAGTGACTTCTACCCGGGAGCCGTGACA |
| | | GTGGCCTGGAAGGCAGATAGCAGCCCCGTCAAGGCGGGAGTGGAGACCAC |
| | | CAAACCCTCCAAACAGAGCAACAACAAGTACGCGGCCAGCAGCTACCTGA |
| | | GCCTGACGCCCGAGCAGTGGAAGTCCCAGAGAAGCTAGAGGTGCCAGGTC |
| | | ACGCATGAAGGGAGCACCGTGGAGAAGACAGTGGCCCCTACAGAATGTTG |
| | | A |
| 366 | Anti CD122 P2C9 Fab | CAGGTGGAGCTGCAGGAGTCGGGCCCAGGAGTGGTGAAGCGTTGGGGGAC |
| | HC (VH, joint CH1) | CGTGTCCCTCACCTGGGCTGTCTCTGGTGGCTCCATCAGCAGTAGTAACT |
| | | GGTGGAGTTGGGTCCGCCAGCCCCCAGGGAAGGGGCTGGAGTGGATTGGG |
| | | GAAATCTATCATAGTGGGAGCACCAACTACAACCCGTCCCTCAAGAGTCG |
| | | AGTCACCATATCAGTAGACAAGTCCAAGAACCAGTTCTCCCTGAAGCTGA |
| | | GCTCTGTGACCGCCGCGGACACGGCCGTGTATTACTGTGTCAGAGAAGGG |
| | | GGCTTACGGGAAGAGCACTGGGGCCAGGGCACCCTGGTCACCGTCTCAAG |
| | | CGCCTCCACCAAGGGCCCATCGGTCTTCCCCCTGGCACCCTCGTCCAAGA |
| | | GCACCTCTGGGGGGACAGGGGCCCTGGGCTGCCTGGTCAAGGACTACTTC |
| | | CCCGAACCGGTGACGGTGTCGTGGAACTCAGGCGCCCTGACCAGCGGGGT |
| | | CCACAGCTTCCCGGCTGTCCTACAGTCCTGAGGAGTCTAGTCCCTCAGCA |
| | | GCGTAGTGACCGTGCCCTCCAGCAGCTTGGGCACCCAGACCTACATCTGC |
| | | AACGTGAATCACAAGCCCAGCAACACCAAGGTGGACAAGAAAGTTGAGCC |
| | | CAAATCTTGT |
| 367 | Anti CD 122 P2C9 | CAGGTCCAGCTGCAGGAGTCCGGGCCAGGGCTGGTGAAACCAAGCGAAAG |
| | scFv and Fc with | AGTGAGTCTGACATGTACCGTGAGTGGGGGGTCCATTAACAATAGTAACT |
| | knob modification | ACTATTGGTCATGGATCAGACAGAGCCCTGGAAGAGGCCTGGAGTGGATC |
| | | GGGGGGATCTACTTCAGCGGCACCACATACTATAACCCATCACTGCAGAG |
| | | CCGGGTGACTATCTCCATTGACACCTCTAAGAATCAGTTCAGCCTGAAGC |
| | | TGAGCAGCGTGACCGCCGCTGATACAGCCATCTACTATTGCGTCCGGCAG |
| | | ATGAATTACTATCACCTGGGCTCTAGTGTGGGGTTCGACCCCTGGGGACA |
| | | GGGAGCACTGGCCACCGTGTCAAGCGTCTCCTCTGGAGGAGGAGGCAGCG |
| | | GCGGAGGAGGCTCTGGAGGAGGGGGGAGTGATGTGGTCATGACACAGAGC |
| | | CCAGCTACTCTCTCTGTGAGTCCCGGCGAAAGGGCCACACTGAGCTGTCG |
| | | CGCTTCACAGAGCGTCAGTTCAAACCTGGCATGGTACCAGCAGAAGCCAG |
| | | GACAGGCACCTTCCCTGCTGATCTATGAGGCTTCTACACGAGCAACTGGC |
| | | ATTCCTGCTAGATTCTCCGGCTCTGGGAGTGGAACCGACTTTACTCTGAC |
| | | CATCAGCTCCCTGCAGAGCGAAGATTTTGCAATCTACTATTGTCAGCAGT |
| | | ATAACGATTGGCTGTGGACCTTCGGGCAGGGGACTAAAGTGGAGATTCGG |
| | | AACAGCGGGGGGGGCACCGCGGCCGCGACTCACACATGCCCACCGTGCCC |
| | | AGCACCTGAAGCCGGGGGGGGACCGTCAGTCTTCCTCTTCCCCCCAAAAC |
| | | CCAAGGACACCCTCATGATCTCCCGGACCCCTGAGGTCACATGCGTGGTG |
| | | GTGGACGTGAGCCACGAAGACCCTGAGGTCAAGTTCAACTGGTACGTGGA |
| | | CGGCGTGGAGGTGCATAATGCCAAGACAAAGCCGCGGGAGGAGCAGTACA |
| | | ACAGCACGTACCGTGTGGTCAGCGTCCTCACCGTCCTGCACCAGGACTGG |
| | | CTGAATGGCAAGGAGTACAAGTGCAAGGTCTCCAACAAAGCCCTCCCAGC |
| | | CCCCATCGAGAAAACCATCTCCAAAGCCAAAGGGCAGCCCCGAGAACCAC |
| | | AGGTGTACACCCTGCCCCCATGCCGGGATGAGCTGACCAAGAACCAGGTC |
| | | AGCCTGTGGTGCCTGGTCAAAGGCTTCTATCCCAGCGACATCGCCGTGGA |
| | | GTGGGAGAGCAATGGGCAGCCGGAGAACAACTACAAGACCACGCCTCCCG |
| | | TGCTGGACTCCGACGGCTCCTTCTTCCTCTACAGCAAGCTCACCGTGGAC |
| | | AAGAGCAGGTGGCAGCAGGGGAACGTCTTCTCATGCTCCGTGATGCATGA |
| | | GGCTCTGCACAACCACTACACGCAGAAGAGCCTCTCCCTGTCTCCGGGTA |
| | | AA |
| 368 | Anti CD122 P2C10 | GATGTTGTGATGACTCAGTCTCCACTCTCCGTGCCCGTCACCCCTGGAGA |
| | Fab LC (VL, joint CL) | GCCGGCCTCCATCTCCTGCAGGTCTAGTCAGAGGCTCCTGCATAGTAATG |
| | | GATACAACTATGTGGATTGGTACCTGCAGAAACCAGGGCAGTCTCCACAG |
| | | CTCCTGATGTATTTGGGTTCTAATCGGGCCTCCGGGGTCCCTGACAGGTT |
| | | CAGTGGCAGTGGATCAGGCACAGATTTTACACTGAAAATCAGCAGAGTGG |
| | | AGGCTGAGGATGTTGGGGTTTATTACTGCATGCAAGGTACACACTGGCCG |
| | | TGGACGTTCGGCCAAGGGACCAAGGTGGAAATCAAACGAACTGTGGCTGC |
| | | ACCATCTGTCTTCATCTTCCCGCCATCTGATGAGCAGTTGAAATCTGGAA |
| | | CTGCCTCTGTTGTGTGCCTGCTGAATAACTTCTATCCCAGAGAGGCCAAA |
| | | GTACAGTGGAAGGTGGATAACGCCCTCCAATCGGGTAACTCCCAGGAGAG |
| | | TGTCACAGAGCAGGACAGCAAGGACAGCACCTACAGCCTCAGCAGCACCC |
| | | TGACGCTGAGCAAAGCAGACTACGAGAAACACAAAGTCTACGCCTGCGAA |
| | | GTCACCCATCAGGGCCTGAGCTCGCCCGTCACAAAGAGCTTCAACAGGGG |
| | | AGAGTGT |
| 369 | Anti CD122 | CAGGTGCAGCTACAGCAGTGGGGCGCAGGACTGTTGAAGCCTTCGGAGAC |
| | P2C10 Fab HC | CCTGTCCCTCACCTGCGCTGTCTATGGTGGGTCCTTCAGTGGTTACTACT |
| | (VH, joint CH1) | GGAGCTGGATCCGCCAGCCCCCAGGGAAGGGGCTGGAGTGGATTGGGGAA |
| | | ATCAATCATAGTGGAAGCACCAACTACAACCCGTCCCTCAAGAGTCGAGT |
| | | CACCATATCAGTAGACACGTCCAAGAACCAGTTCTCCCTGAAGCTGAGCT |
| | | CTGTGACCGCCGCGGACACGGCTGTGTATTACTGTGCGAGAGGCACGGAT |
| | | ACAGCTATGGCTGACTACTGGGGCCAGGGAACCCTGGTCACCGTCTCAAG |
| | | CGCCTCCACCAAGGGCCCATCGGTCTTCCCCGTGGGACCCTCCTCCAAGA |
| | | GCACCTCTGGGGGCACAGCGGCCCTGGGCTGCCTGGTCAAGGACTACTTC |
| | | CCCGAACCGGTGACGGTGTCGTGGAACTCAGGCGCCCTGACCAGCGGCGT |
| | | CCACACCTTCCCGGCTGTCCTACAGTCCTCAGGACTCTACTCCCTCAGCA |
| | | GCGTAGTGACCGTGCCCTCCAGCAGCTTGGGCACCCAGACCTACATCTGC |
| | | AACGTGAATCACAAGCCCAGCAACACCAAGGGGACAAGAAAGTTGAGCCC |
| | | AAATCTTGT |
| 370 | Anti CD122 P2C10 | CAGGTCCAGCTGCAGGAGTCCGGGCCAGGGCTGGTGAAACCAAGCGAAAC |
| | scFv and Fc with | ACTGAGTCTGACATGTACCGTGAGTGGGGGGTCCATTAACAATAGTAACT |
| | knob modification | ACTATTGGTCATGGATCAGACAGAGCCCTGGAAGAGGCCTGGAGTGGATC |
| | | GGGGGGATCTACTTCAGCGGCACCACATACTATAACCCATCACTGCAGAG |
| | | CCGGGTGACTATCTCCATTGACACCTCTAAGAATCAGTTCAGCCTGAAGC |
| | | TGAGCAGCGTGACCGCCGCTGATACAGCCATCTACTATTGCGTCCGGCAG |
| | | ATGAATTACTATCACCTGGGCTCTAGTGTGGGGTTCGACCCCTGGGGACA |
| | | GGGAGCACTGGCCACCGTGTCAAGCGTCTCCTCTGGAGGAGGAGGCAGCG |
| | | GCGGAGGAGGCTCTGGAGGAGGGGGAGTGATGTGGTCATGACACAGAGCC |
| | | CAGCTACTCTGTGTGTGAGTCCCGGCGAAAGGGCCACACTGAGCTGTCGC |
| | | GCTTCACAGAGCGTCAGTTCAAACCTGGCATGGTACCAGCAGAAGCCAGG |
| | | ACAGGCACCTTCCCTGCTGATCTATGAGGCTTCTACACGAGCAACTGGCA |
| | | TTCCTGCTAGATTCTCCGGCTCTGGGAGTGGAACCGACTTTACTCTGACC |
| | | ATCAGCTCCCTGCAGAGCGAAGATTTTGCAATCTACTATTGTCAGCAGTA |
| | | TAACGATTGGCTGTGGACCTTCGGGGAGGGGACTAAAGTGGAGATTCGGA |
| | | ACAGCGGGGGGGGCACCGCGGCCGCGACTCACACATGCCCACCGTGCCCA |
| | | GCACCTGAAGCCGGGGGGGGACCGTCAGTCTTCCTCTTCCCCCCAAAACC |
| | | CAAGGACACCCTCATGATCTCCCGGACCCCTGAGGTCACATGCGTGGTGG |
| | | TGGACGTGAGCCACGAAGACCCTGAGGTCAAGTTCAACTGGTACGTGGAC |
| | | GGCGTGGAGGTGCATAATGCCAAGACAAAGCCGCGGGAGGAGCAGTACAA |
| | | CAGCACGTACCGTGTGGTCAGCGTCCTCACCGTCCTGCACCAGGACTGGC |
| | | TGAATGGCAAGGAGTACAAGTGCAAGGTCTCCAACAAAGCCCTCCCAGCC |
| | | CCCATCGAGAAAACCATGTCCAAAGCCAAAGGGCAGCCCCGAGAACCACA |
| | | GGTGTACACCCTGCCCCCATGCCGGGATGAGCTGACCAAGAACCAGGTCA |
| | | GCCTGTGGTGCCTGGTCAAAGGCTTCTATCCCAGCGACATCGCCGTGGAG |
| | | TGGGAGAGCAATGGGCAGCCGGAGAACAACTACAAGACCACGCCTCCCGT |
| | | GCTGGACTCCGACGGCTCCTTCTTCCTCTACAGCAAGCTCACCGTGGACA |
| | | AGAGCAGGTGGCAGCAGGGGAACGTCTTCTCATGCTCCGTGATGCATGAG |
| | | GCTCTGCACAACCACTACACGCAGAAGAGCCTCTCCCTGTCTCCGGGTAA |
| | | A |
| 371 | Anti CD122 P2C11 | GACATCCAGATGACCCAGTCTCCACCCTCCCTGTCCGCATCTGTAGGAGA |
| | Fab LC (VL, joint CL) | CAGAGTCACCATCACTTGTCAGGCGAGTCAGGACATTAACAACTATTTGA |
| | | ATTGGTATCACCAAAAACCAGGGAAGGCCCCTGAGCTCCTGATCTACGAT |
| | | GCATCTCAGTTGGAAACAGGGGTCCCATCAAGGTTCAGTGGAAGTGGATC |
| | | TGGGACAGAGTTTACTTTCATCATCAGCAGCCTGCAGCCTGAAGATACCG |
| | | GTACATATTACTGTCAACAATATGATTGGCTCCCCCTTTCTTACGGCGGA |
| | | GGGACCAAGGTTGAGATCAAACGAACTGTGGCTGCACCATCTGTCTTCAT |
| | | CTTCCCGCCATCTGATGAGCAGTTGAAATCTGGAACTGCCTCTGTTGTGT |
| | | GCCTGCTGAATAACTTCTATCCCAGGGAGGCCAAAGTACAGTGGAAGGTG |
| | | GATAACGCCCTCCAATCGGGTAACTCCCAGGAGAGTGTCACAGAGCAGGA |
| | | CAGCAAGGACAGCACCTACAGCCTCAGCAGCACCCTGACGCTGAGCAAAG |
| | | CAGACTACGAGAAACACAAAGTCTACGCCTGCGAAGTCACCCATCAGGGC |
| | | CTGAGCTCGCCCGTCACAAAGAGCTTCAACAGGGGAGAGTGT |
| 372 | Anti CD122 P2C11 | CAGGTACAGCTGCAGCAGTCAGGTCCAGGACTGGTGAAGCCCTCGCAGAC |
| | Fab HC (VH, joint CH1) | CCTCTCACTCACCTGTGCCATCTCCGGGGACAGTGTCTCTGGCAACAGTG |
| | | CTACTTGGAACTGGATCAGGCAGTCCCCATCGCGAGGCCTTGAGTGGCTG |
| | | GGAAGGACATATTACAGGTCCAAGTGGAATCATGATTATGCAGAATGTGT |
| | | GAAAAGTCGAATAACCATCAACCCAGACACATCCAAGAACCAGTTCTCCC |
| | | TGCAGCTGAACTCTGTGACTCCCGAGGACACGGCTGTCTATTACTGTGCA |
| | | AGAGACTCCAAGTCTGCTTTTGATATCTGGGGCCAAGGGACAATGGTCAC |
| | | CGTCTCAAGCGCCTCCACCAAGGGCCCATCGGTCTTCCCCCTGGCACCCT |
| | | CCTCCAAGAGCACCTCTGGGGGCACAGCGGCCCTGGGCTGCCTGGTCAAG |
| | | GACTACTTCCCCGAACCGGTGACGGTGTCGTGGAACTCAGGCGCCCTGAC |
| | | CAGCGGCGTCCACACCTTCCCGGCTGTCCTACAGTCCTCAGGACTCTACT |
| | | CCCTCAGCAGCGTAGTGACCGTGCCCTCCAGCAGCTTGGGCACCCAGACC |
| | | TACATCTGCAACGTGAATCACAAGCCCAGCAACACCAAGGTGGACAAGAA |
| | | AGTTGAGGCCAAATCTTGT |
| 373 | Anti CD122 P2C11 | CATGTGCAGCTGGTGGAGACTGGAGGGGGACTGGTGCAGCCTGGGGGGTC |
| | scFv and Fc with | ACTGAGACTGAGTTGTGCCGCTTCTGGGTTCACTTTCAGCTCCTACGCAA |
| | knob modification | TGAGCTGGGTGCGGCAGGCCCCCGGAAAAGGCCTGGAGTGGGTCTCCGCC |
| | | ATCAGTGGATCAGGCGGGAGCACCTACTATGCTGACTCCGTGACAGGCCG |
| | | GTTCACTATTAGCAGAGATAACTCCAAGAATACCCTGTACCTCCAGATGA |
| | | ACTCCCTGAGGGGCGAAGACACAGGTGTGTACTATTGCGCTCGCGACCTG |
| | | GGCGATTATTGGGGGCAGGGAACACTGGTGACTGTCTCTAGTGGAGGAGG |
| | | AGGATCTGGAGGAGGAGGCAGTGGAGGAGGCGGGTCAGACATCCAGCTGA |
| | | CTCAGTCTCCTTCAAGCCTGAGCGCATCCATGGGGGACCGAGTCACCATC |
| | | ACATGTCAGGCCAGCCAGGATATTGGCAACTACCTGAATTGGTATCAGCT |
| | | GAAGCCCGGCAAGGCTCCTAAGCTGCTGATTCTACGACGCATCTAATCTG |
| | | GAGACAGGGGTGCCAAGTAGATTCTCTGGCAGTGGGTCAGGAACTGATTT |
| | | CACCTTCACCATCAGCAGCCTCCAGCCAGAGGACATTGCCACATACTATT |
| | | GCCTCCAGCTGTACGATTATCCCCTGACCTTTGGAGGGGGGACAAAAGTG |
| | | GAAATCAAGAACAGCGGCGCGGGCACCGCGGCCGCGACTCACACATGCCC |
| | | ACCGTGCCCAGCACCTGAAGCCGGGGGGGGACCGTCAGTCTTCCTCTTCC |
| | | CCCCAAAACCCAAGGACACCCTCATGATCTCCCGGACCCCTGAGGTCACA |
| | | TGCGTGGTGGTGGACGTGAGCCACGAAGACCCTGAGGTCAAGTTCAACTG |
| | | GTACGTGGACGGCGTGGAGGTGCATAATGCCAAGACAAAGCCGCGGGAGG |
| | | AGCAGTACAACAGCACGTACCGTGTGGTCAGCGTCCTCACCGTCCTGCAC |
| | | CAGGACTGGCTGAATGGCAAGGAGTACAAGTGCAAGGTCTCCAACAAAGC |
| | | CCTCCCAGCCCCCATCGAGAAAACCATCTCCAAAGCCAAAGGGCAGCCCC |
| | | GAGAACCACAGGTGTACACCCTGCCCCCATGCCGGGATGAGCTGACCAAG |
| | | AACCAGGTCAGCCTGTGGTGCCTGGTCAAAGGCTTCTATCCCAGCGACAT |
| | | CGCCGTGGAGTGGGAGAGCAATGGGCAGCCGGAGAACAACTACAAGACCA |
| | | CGCCTCCCGTGCTGGACTCCGACGGCTCCTTCTTCCTCTACAGCAAGCTC |
| | | ACCGTGGACAAGAGCAGGTGGCAGCAGGGGAACGTCTTCTCATGCTCCGT |
| | | GATGGATGAGGCTCTGCACAACCACTAGACGCAGAAGAGCGTGTCCCTGT |
| | | CTCGGGGTAAA |
| 374 | Anti CD122 P2E6 | AATTTTATGCTGACTCAGCCCCACTCTGTGTCGGGGTCTCCGGGGAAGAC |
| | Fab LC (VL, joint CL) | GATAACCATCTCCTGCACCCGCAGCAGTGGCAACTTTGCCAGCACCTATG |
| | | TGCAGTGGTACCAACAGCGCCCGGGCAGTTCCCCCGCCATTGTGATCTAT |
| | | GACGATGATCAACGACCCTCTGGTGTCCCTGACCGCTTCTCTGGCTCCAT |
| | | CGACAGGTCCTCCAACTCTGCCTCCCTCACCATCTCTGGACTGGAGACTG |
| | | AGGACGAGGCTGACTACTATTGTCAGTCTTATGATAGCAGCAATTTTTGG |
| | | GTGTTCGGCGGAGGGACCAAACTGACCGTCCTAGGTCAGCCCAAGGCTGC |
| | | CCCCTCGGTCACTCTGTTCCCACCCTCGTCTGAGGAGCTTGAAGCCAAGA |
| | | AGGCCACAGTGGTGTGTGTGATAAGTGACTTCTACCCGGGAGCCGTGAGA |
| | | GTGGCCTGGAAGGCAGATAGCAGCCCCGTCAAGGCGGGAGTGGAGACCAC |
| | | CACAGCCTCCAAACAAAGCAACAAGAAGTACGGGGCCAGCAGCTACCTGA |
| | | GCCTGACGCCTGAGCAGTGGAAGTCCCACAAAAGCTACAGCTGCCAGGTC |
| | | ACGCATGAAGGGAGCACGGTGGAGAAGAGAGTGGCCCCTACAGAATGTTC |
| | | A |
| 375 | Anti CD122 P2E6 | CAGCTGCAGCTGCAGGAGTCGGGCCCAGGACTGGTGAAGCCTTCGGAGAC |
| | Fab HC (VH, joint CH1) | CGTGTCCCTCACCTGCAGTGTCTTTGGTGTCTCCATCACCAGTGGTAGTT |
| | | GGTGGAGTTGGGTCCGCCAGTCCCCAGGGAAGGAGCTGGAGTGGATAGGC |
| | | GAAATCTATCATAATGGGAACACCAACTACAACCCGTCCCTCAAGAGTCG |
| | | AGTCACCATATCGGTTGACACGTCCAAGAACCAGTTCTCCCTGAAACTGA |
| | | GCTGTGTGACCGCCGCAGACACGGCTGTCTATTACTGTGTCTCCGGATTT |
| | | GACTACTGGGGCCAGGGAACCCTGGTCACCGTCTCAAGCGCCTCCACCAA |
| | | GGGCCCATCGGTCTTCCCCCTGGCACCCTCCTCCAAGAGCACCTCTGGGG |
| | | GCACAGCGGCCCTGGGCTGCCTGGTCAAGGACTACTTCCCCGAACCGGTG |
| | | ACGGTGTCGTGGAACTCAGGCGCCCTGACCAGCGGCGTCCACACCTTCCC |
| | | GGCTGTCCTACAGTCCTCAGGACTCTACTCCCTCAGCAGCGTAGTGAGCG |
| | | TGCCCTCCAGCAGCTTGGGCACCCAGACCTACATCTGCAACGTGAATCAC |
| | | AAGCCCAGCAACACCAAGGTGGACAAGAAAGTTGAGCCCAAATCTTGT |
| 376 | Anti CD122 P2E6 scFv | CAGCTGCAGCTGCAGGAGAGCGGCCCCGGACTGGTGAAGCCTAGCGAAAC |
| | and Fc with knob | CCTGAGCCTGACTTGTTCTGTCTTTGGAGTGAGCATCACTTCTGGAAGTT |
| | modification | GGTGGAGCTGGGTGAGACAGTCCCCCGGCAAGGAGCTGGAATGGATCGGG |
| | | GAAATCTACCACAACGGAAATACAAACTATAATCCTTCCCTGAAATCTCG |
| | | GGTGACTATCAGTGTCGATACCTCAAAGAACCAGTTCAGCCTGAAGCTGA |
| | | GCAGCGTGACCGCCGCTGATACAGCCGTGTACTATTGCGTCAGCGGCTTT |
| | | GACTACTGGGGCCAGGGGACTCTGGTGACCGTCTCTAGTGGAGGAGGAGG |
| | | CTCTGGAGGAGGAGGGAGTGGAGGAGGAGGCAGCAACTTCATGCTGACCC |
| | | AGCCTCATTCAGTGAGCGGCAGCCCCGGCAAGACCATCACAATTTCTTGT |
| | | ACCCGCTCAAGCGGGAATTTTGCTAGCACATACGTGCAGTGGTATCAGCA |
| | | GCGACCCGGCTCCTCTCCTGCAATCGTGATCTACGACGATGACCAGCGAC |
| | | CAAGCGGCGTCCCCGATAGATTCTCTGGGAGTATCGACAGGAGTTCAAAC |
| | | TCAGCAAGCCTGACAATTAGCGGCCTGGAGACTGAAGATGAGGCCGACTA |
| | | CTATTGCCAGTCCTATGACAGCTCCAATTTCTGGGTGTTTGGCGGGGGAA |
| | | CAAAACTGACTGTCCTGAACAGCGGCGCGGGCACCGCGGCCGCGACTCAC |
| | | ACATGCCCACCGTGCCCAGCACCTGAAGCCGCGGGGGGACCGTCAGTCTT |
| | | CCTCTTCCCCCCAAAACCCAAGGACACCCTCATGATCTCCCGGACCCCTG |
| | | AGGTCACATGCGTGGTGGTGGACGTGAGCCACGAAGACCCTGAGGTCAAG |
| | | TTCAACTGGTACGTGGACGGCGTGGAGGTGCATAATGCCAAGACAAAGCC |
| | | GCGGGAGGAGCAGTACAACAGCACGTACCGTGTGGTCAGCGTCCTCACCG |
| | | TCCTGCACCAGGACTGGCTGAATGGGAAGGAGTACAAGTGCAAGGTCTCC |
| | | AACAAAGCCCTCCCAGCCCCCATCGAGAAAACCATCTCCAAAGCCAAAGG |
| | | GCAGCCCCGAGAACCACAGGTGTACACCCTGCCCCCATGCCGGGATGAGC |
| | | TGACCAAGAACCAGGTCAGCCTGTGGTGCCTGGTCAAAGGCTTCTATCCC |
| | | AGCGACATCGCCGTGGAGTGGGAGAGCAATGGGCAGCCGGAGAACAACTA |
| | | GAAGACCACGCCTCCCGTGCTGGACTCCGACGGCTCCTTCTTCCTCTACA |
| | | GCAAGCTCACCGTGGACAAGAGCAGGTGGCAGCAGGGGAACGTCTTCTGA |
| | | TGCTCCGTGATGCATGAGGCTCTGCACAACCACTACACGCAGAAGAGCGT |
| | | CTCCCTGTCTCCGGGTAAA |
| 377 | Anti CD122 P2E11 | GAAATTGTGTTGACGCAGTCTCCATCCTCCGTGTCTGCATCTGTAGGAGA |
| | Fab LC (VL, joint CL) | CAGAGTCACCATCACTTGCCAGGCGAGTCAGGACATTAATAATTATTTAA |
| | | ATTGGTATCAGCAGAAACCAGGGAAAGCCCCTAAGCTCCTGATCTACGAT |
| | | GCATCCAATTTGGAAACAGGGGTCCCATCAAAGTTCAGTGGAAGTGGATC |
| | | TGGGACAGATTTTACTTTCACCATCAGCAGCCTGCAGCCTGAAGATATTG |
| | | CAACATATTACTGTCAACAGTATGCCAATCTCCCCTCTTTTGGCCAGGGG |
| | | ACCAAGCTGGAGATCAAACGAACTGTGGCTGCACCATGTGTCTTCATCTT |
| | | CCCGCCATCTGATGAGCAGTTGAAATCTGGAACTGCCTCTGTTGTGTGCC |
| | | TGCTGAATAACTTCTATCCCAGAGAGGCCAAAGTACAGTGGAAGGTGGAT |
| | | AAGGCCCTCCAATCGGGTAACTCCCAGGAGAGTGTCACAGAGCAGGACAG |
| | | CAAGGACAGCACCTACAGCCTCAGCAGCACCCTGACGCTGAGCAAAGCAG |
| | | ACTAGGAGAAACACAAAGTCTACGCCTGCGAAGTCACCCATCAGGGCCTG |
| | | AGCTCGCCCGTCACAAAGAGCTTCAACAGGGGAGAGTGT |
| 378 | Anti CD122 P2E11 | CAGGTGCAGCTGCAGGAGTCCGGCCCAGGACTGGTGAAGCCTTCGGAGAC |
| | Fab HC (VH, joint CH1) | CCTGTCTCTCACCTGCACTGTCTCTGGTGTCTCCATCAGCAGTAGAAGTG |
| | | ACCACTGGGGCTGGGTCCGCCAGCCCCCAGGGAAGGGGCTGGAGTGGATT |
| | | GGAAGTATCTCTTATAGTGGGAGCACCTACTACAACCCGTCCCTCAAGAG |
| | | CCGAGTCACCATATCCGTAGACACCTCCAAGAACCAACTCTCCCTGAAGC |
| | | TGAGCTCTGTGACCGCCGCAGACACGGCTGTGTATTACTGTGCGAGAGAG |
| | | TCGCACCCAGCAGCTGCACTGGTTGGGTGGGGCCAGGGCACCCTGGTCAC |
| | | CGTCTCAAGCGCCTCCACCAAGGGCCCATCGGTGTTCCCCCTGGCACCCT |
| | | CCTCCAAGAGCACCTCTGGGGGCACAGCGGCCCTGGGCTGCCTGGTCAAG |
| | | GACTACTTCCCCGAACCGGTGACGGTGTCGTGGAACTCAGGCGCCCTGAC |
| | | CAGCGGCGTCCACACCTTCCCGGCTGTCCTACAGTCCTCAGGACTCTACT |
| | | CCCTCAGCAGCGTAGTGACCGTGCCCTCCAGCAGCTTGGGCACCCAGACC |
| | | TACATCTGCAACGTGAATCACAAGCCCAGCAACACCAAGGTGGACAAGAA |
| | | AGTTGAGCCCAAATCTTGT |
| 379 | Anti CD122 P2E11 | CAGGTGCAGCTGCAGGAAAGCGGACCCGGACTGGTGAAGCCTAGCGAGAC |
| | scFv and Fc with | TCTGAGCCTGACTTGTACCGTGAGCGGCGTGAGCATTAGCTCCCGGAGCG |
| | knob modification | ACCAGTGGGGATGGGTGAGACAGCCCCCTGGCAAAGGGCTGGAGTGGATC |
| | | GGGAGCATTTCCTACTCTGGAAGTACTTACTATAACCCCTCACTGAAGAG |
| | | CAGGGTGACTATCTCCGTGGACACCTCTAAAAATCAGCTGTCTCTGAAGC |
| | | TGTCTAGTGTGACCGCCGCTGATACAGCAGTCTACTATTGCGCCCGCGAG |
| | | TCCCATCCTGCCGCCGCCCTGGTGGGATGGGGAGAGGGGACACTGGTGAC |
| | | TGTCTCAAGCGGAGGAGGAGGCAGTGGAGGAGGAGGGTCAGGAGGGGGGG |
| | | GAAGCGAAATCGTCCTGACACAGAGTCCATCCTCTCTGTCAGCCAGCGTG |
| | | GGCGACCGAGTCACCATCACATGTCAGGCCTCCCAGGATATTAACAATTA |
| | | CCTGAACTGGTATCAGCAGAAGCCAGGCAAAGCTCCCAAGCTGCTGATCT |
| | | ACGATGCATCCAATCTGGAAACAGGGGTGCCCTCTAAATTCTCCGGATCT |
| | | GGCAGTGGGACTGACTTCACCTTCACCATCAGCAGCCTCCAGCCTGAGGA |
| | | TATTGCCACCTACTATTGCCAGCAGTATGCTAACCTGCCCAGCTTCGGAC |
| | | AGGGCACAAAACTGGAAATTAAGAACAGCGGCGCGGGCACCGCGGCCGCG |
| | | ACTCACACATGCCCACCGTGCCCAGCACCTGAAGCCGGGGGGGGACCGTC |
| | | AGTCTTCCTCTTCCCCCCAAAACCCAAGGACACCCTCATGATCTCCCGGA |
| | | CCCCTGAGGTCACATGCGTGGTGGTGGACGTGAGCCACGAAGACCCTGAG |
| | | GTCAAGTTCAACTGGTACGTGGACGGCGTGGAGGTGCATAATGCCAAGAC |
| | | AAAGCCGGGGGAGGAGCAGTACAACAGCACGTACCGTGTGGTCAGCGTCC |
| | | TCACCGTCCTGCACCAGGACTGGCTGAATGGCAAGGAGTACAAGTGCAAG |
| | | GTCTCCAACAAAGCCCTCCCAGCCCCCATCGAGAAAACCATCTCCAAAGC |
| | | CAAAGGGCAGCCCCGAGAACCACAGGTGTACACCCTGCCCCCATGCCGGG |
| | | ATGAGCTGACCAAGAACCAGGTCAGCCTGTGGTGCCTGGTCAAAGGCTTC |
| | | TATCCCAGCGACATCGCCGTGGAGTGGGAGAGCAATGGGCAGCCGGAGAA |
| | | CAACTACAAGACCACGCCTCCCGTGGTGGACTCCGACGGCTCCTTCTTCC |
| | | TCTACAGCAAGCTCACCGTGGACAAGAGCAGGTGGCAGCAGGGGAACGTC |
| | | TTCTCATGCTCCGTGATGCATGAGGCTCTGCACAACCACTACACGCAGAA |
| | | GAGCCTCTCCCTGTCTCCGGGTAAA |
| 380 | Anti CD122 P2F9 | GAAATTGTGCTGACTCAGTCTCCACTCTCCCTGCCCGTCACCCCTGGAGA |
| | Fab LC (VL, joint CL) | GCCGGCCTCCATCTCCTGCAGGTCTAGTCAGAGCCTCCTGCATAGTAATG |
| | | GATACAACTATTTGGATTGGTACCTGCAGAAGCCAGGGCAGTCTCCACAG |
| | | CTCCTGATCTATTTGGGTTCTAATCGGGCCTCGGGGGTCCCTGACAGGTT |
| | | CAGTGGCAGTGGATCAGGCACAGATTTTACACTGAGCATCAGCAGAGTGG |
| | | AGGCTGAGGATGTTGGGGTTTATTACTGCATGCAAGCTCTACAAACTCCT |
| | | CCGACGTTCGGCCAAGGGACCAAGGTGGAAATCAAACGAACTGTGGCTGC |
| | | ACCATCTGTCTTCATCTTCCCGCCATCTGATGAGCAGTTGAAATCTGGAA |
| | | CTGCCTCTGTTGTGTGCCTGCTGAATAACTTCTATCCCAGAGAGGCCAAA |
| | | GTACAGTGGAAGGTGGATAACGCCCTCCAATCGGGTAACTCCCAGGAGAG |
| | | TGTCACAGAGCAGGACAGCAAGGACAGCACCTACAGCCTCAGCAGCACCC |
| | | TGACGCTGAGCAAAGCAGACTACGAGAAACACAAAGTCTACGCCTGCGAA |
| | | GTCACCCATCAGGGCCTGAGCTCGCCCGTCACAAAGAGCTTCAACAGGGG |
| | | AGAGTGT |
| 381 | Anti CD122 P2F9 | GAGGTCCAGCTGGTACAGTCTGGGGCTGAGGTGAAGAAGCCTGGGTCGTC |
| | Fab HC (VH, joint CH1) | GGTGAAGGTCTCCTGCAAGGCTTCTGGTTACACCTTTACCAGCTATGGTA |
| | | TCAGCTGGGTGCGACAGGCCCCTGGACAAGGGCTTGAGTGGATGGGATGG |
| | | ATCAGCGCTTACAATGGTAACACAAACTATGCACAGAAGCTCCAGGGCAG |
| | | AGTCACCATGACCACAGACACATCCACGAGCACAGCCTACATGGAGCTGA |
| | | GCAGCCTGAGATCTGAGGACACGGCCGTGTATTACTGTGCGAGAGCCCCT |
| | | GACTACCGTGACTCCTCCAACTACTACTACTACTACATGGACGTCTGGGG |
| | | CAAAGGGACCACGGTCACCGTGTCAAGCGCCTCCACCAAGGGCCCATCGG |
| | | TCTTCCCCCTGGCACCCTCCTCCAAGAGCACCTCTGGGGGCACAGCGGCC |
| | | CTGGGCTGCCTGGTCAAGGACTACTTCCCCGAACCGGTGACGGTGTCGTG |
| | | GAACTCAGGCGCCCTGACCAGCGGCGTCCACACCTTCCCGGCTGTCCTAC |
| | | AGTCCTCAGGACTCTACTCCCTCAGCAGCGTAGTGACCGTGCCCTCCAGC |
| | | AGCTTGGGCACCCAGACCTACATCTGCAACGTGAATCACAAGCCCAGCAA |
| | | CACCAAGGTGGACAAGAAAGTTGAGCCCAAATCTTGT |
| 382 | Anti CD122 P2F9 | GAAGTGCAGCTGGTGCAGAGGGGGGCAGAGGTGAAAAAACCTGGGTCATC |
| | soFy and Fe with | CGTCAAAGTCTCCTGTAAGGCAAGCGGCTACACATTTACTTCATACGGCA |
| | knob modification | TCAGCTGGGTGCGACAGGCCCCTGGCCAGGGGCTGGAGTGGATGGGATGG |
| | | ATTAGCGCATATAACGGCAATACAAACTACGCCCAGAAGCTCCAGGGGAG |
| | | AGTGACTATGACCACAGACACAAGTACTTCAACCGCCTATATGGAGCTGA |
| | | GCAGGCTGAGGTCCGAAGATAGCGCTGTGTACTATTGCGCCCGCGCTCCT |
| | | GACTACGGCGATTCTAGTAACTACTACTACTACTACATGGACGTCTGGGG |
| | | AAAAGGCACTACCGTGACAGTCTCAAGCGGCGGAGGAGGCTCCGGAGGAG |
| | | GAGGGTCTGGAGGAGGAGGAAGCGAGATCGTGCTGACTCAGTCTCCACTG |
| | | AGTCTGCCAGTCACCCCCGGCGAACCTGCAAGCATTTCCTGTCGGTCCTC |
| | | TCAGTCCCTGCTGCACTCTAATGGGTATAACTACCTGGACTGGTACTTGC |
| | | AGAAGCCAGGACAGTCTCCCCAGCTGCTGATCTACCTGGGCAGTAACCGA |
| | | GCTAGGGGGGTGCCTGACAGATTCTCTGGGAGTGGATCAGGCACAGATTT |
| | | TACTCTGAGCATCAGCCGGGTGGAGGCTGAAGATGTGGGCGTCTATTACT |
| | | GCATGCAGGCCCTCCAGACCCCCCCTACATTCGGGCAGGGAACCAAGGTG |
| | | GAAATCAAAAACAGCGGGGGGGGCACCGCGGCCGCGACTCACACATGCCC |
| | | ACCGTGCCCAGCACCTGAAGCCGGGGGGGGACCGTCAGTCTTCCTCTTCC |
| | | CCCCAAAACCCAAGGACACCCTCATGATCTCCCGGACCCCTGAGGTCACA |
| | | TGCGTGGTGGTGGACGTGAGCCACGAAGACCCTGAGGTCAAGTTCAACTG |
| | | GTACGTGGACGGCGTGGAGGTGCATAATGCCAAGACAAAGCCGCGGGAGG |
| | | AGCAGTACAACAGCACGTACCGTGTGGTCAGCGTCCTCACCGTCCTGCAC |
| | | CAGGACTGGCTGAATGGCAAGGAGTACAAGTGCAAGGTCTCCAACAAAGC |
| | | CCTCCCAGCCCCCATCGAGAAAACCATCTCCAAAGCCAAAGGGCAGCCCC |
| | | GAGAACCACAGGTGTACACCCTGCCCCCATGCCGGGATGAGCTGACCAAG |
| | | AACCAGGTCAGCCTGTGGTGCCTGGTCAAAGGCTTCTATCCCAGCGACAT |
| | | CGCCGTGGAGTGGGAGAGCAATGGGCAGCCGGAGAACAACTACAAGACCA |
| | | CGCCTCCCGTGCTGGACTCCGACGGCTCCTTCTTCCTCTACAGCAAGCTC |
| | | ACCGTGGACAAGAGCAGGTGGCAGCAGGGGAACGTCTTCTCATGCTCCGT |
| | | GATGCATGAGGCTCTGCACAACCACTACACGCAGAAGAGCCTCTCCGTGT |
| | | CTCCGGGTAAA |
| 383 | Anti CD122 P2F10 | GACATCCAGTTGACCCAGTCTCCATCCTCCCTGTCTGCATCTGTAGGAGA |
| | Fab LC (VL, joint CL) | CAGAGTCACCGTCACTTGCCAGGCGAGCCAGGACATTGGCCACAATTTAA |
| | | ATTGGTATCAGCAGAGACCTGGGAAAGCCCCTCAGCTCCTGATCTACGAT |
| | | GCATCCAATTTGGAAACAGGGGTCCCATCAAGGTTCAGTGGAAGTGGATC |
| | | TGGGACACAATTTACTTTCACCATGAGCAGTCTGCAGCCTGAAGATATTG |
| | | CAACATATTACTGTCAACAATATGATTTTCTCCCTCCTGACTTCGGCCCA |
| | | GGGACCAAAGTGGAGATCAAACGAACTGTGGCTGCACCATCTGTCTTCAT |
| | | CTTCCCGCCATGTGATGAGCAGTTGAAATCTGGAACTGCCTCTGTTGTGT |
| | | GCCTGCTGAATAACTTCTATCCCAGAGAGGCCAAAGTACAGTGGAAGGTG |
| | | GATAACGCCCTCCAATCGGGTAACTCCCAGGAGAGTGTCACAGAGCAGGA |
| | | CAGCAAGGACAGCACCTACAGCCTCAGCAGCACCCTGACGCTGAGCAAAG |
| | | CAGACTACGAGAAACACAAAGTCTACGCCTGCGAAGTCACCCATCAGGGC |
| | | CTGAGCTCGCCCGTCACAAAGAGCTTCAACAGGGGAGAGTGT |
| 384 | Anti CD122 P2F10 | CAGGTCCAGCTGGTGCAGTGTGGAGCTGAGGTGAAGAAGCCTGGGGCCTC |
| | Fab HC (VH, joint CH1) | AGTGAAGGTCTCCTGCAAGGCTTCTGGTTACACCTTTACCAGCTATGGTA |
| | | TCAGCTGGGTGCGACAGGCCCCTGGACAAGGGCTTGAGTGGATGGGATGG |
| | | ATCAGCGCTTACAATGGTAACACAAACTATGCACAGAAGCTCCAGGGCAG |
| | | AGTCACCATGACCACAGACACATCCACGAGCACAGCCTACATGGAGCTGA |
| | | GGAGCCTGAGATCTGACGACACGGGGGTGTATTACTGTGCGAGAGATACC |
| | | TCCGGGGACTATAGCAGTGGCTGGTACCTAGGAGTTCCTTTTGACTACTG |
| | | GGGCCAGGGCACCCTGGTCACCGTCTCAAGCGCCTCCACCAAGGGCCCAT |
| | | CGGTCTTCCCCCTGGCACCCTCCTCCAAGAGCACCTCTGGGGGCACAGCG |
| | | GCCCTGGGCTGCCTGGTCAAGGACTACTTCCCCGAACCGGTGACGGTGTC |
| | | GTGGAACTCAGGCGCCCTGACCAGCGGCGTCCACACCTTCCCGGCTGTCC |
| | | TACAGTCCTCAGGACTCTACTCCCTCAGCAGCGTAGTGACCGTGCCCTCC |
| | | AGCAGCTTGGGCACCCAGACCTACATCTGCAACGTGAATCACAAGCCCAG |
| | | CAACACCAAGGTGGACAAGAAAGTTGAGCCCAAATCTTGT |
| 385 | Anti CD122 P2F10 | GAGGTGCAGCTGGTCGAGAGGGGAGGGGAGGTGAAGAAGCGGGGAGCATC |
| | scFv and | AGTGAAAGTCAGTTGTAAAGCAAGCGGATACACATTTACATCTTACGGCA |
| | Fc with knob | TCAGTTGGGTGCGACAGGCACCAGGCCAGGGGCTGGAGTGGATGGGATGG |
| | modification | ATTTCTGCATACAACGGCAATACAAACTATGCCCAGAAGCTCCAGGGGAG |
| | | AGTCACTATGACCACAGACACTAGTACCTCAACAGCTTACATGGAACTGC |
| | | GGAGCCTGAGATCCGACGATACTGCCGTGTACTATTGCGCTCGGGACACC |
| | | AGCGGCGATTACAGCTCCGGCTGGTATCTGGGGGTCCCCTTCGACTATTG |
| | | GGGACAGGGCACCCTGGTGACAGTCTCTAGTGGGGGGGGAGGCTCAGGAG |
| | | GAGGAGGGAGCGGAGGAGGAGGCAGCGACATCCAGCTGACCCAGAGCCCT |
| | | TCAAGCCTGAGCGCATCCGTGGGCGACAGGGTGACTGTCACCTGCCAGGC |
| | | TTCCCAGGACATCGGGCACAATCTGAACTGGTATCAGCAGCGCCCAGGAA |
| | | AAGCTCCCCAGCTGCTGATCTACGACGCATCTAATCTGGAGACCGGCGTG |
| | | CCCAGTCGCTTTTCTGGGAGTGGATCAGGCACACAGTTCACCTTCACCAT |
| | | CAGCAGCCTCCAGCCTGAGGATATTGCCACTTACTATTGTCAGCAGTATG |
| | | ACTTCCTGCCCCCTGATTTTGGGCCAGGAACCAAGGTGGAGATCAAGAAC |
| | | AGCGGGGGGGGCACCGCGGCCGCGACTCACACATGCCCACCGTGCCCAGC |
| | | ACCTGAAGCCGGGGGGGGACCGTCAGTCTTCCTCTTCCCCCCAAAACCCA |
| | | AGGACACCCTCATGATCTCCCGGACCCCTGAGGTCACATGCGTGGTGGTG |
| | | GACGTGAGCCACGAAGACCCTGAGGTCAAGTTCAACTGGTACGTGGACGG |
| | | CGTGGAGGTGCATAATGCCAAGACAAAGCCGCGGGAGGAGCAGTACAACA |
| | | GCACGTACCGTGTGGTCAGCGTCCTCACCGTCCTGCACCAGGACTGGCTG |
| | | AATGGCAAGGAGTACAAGTGCAAGGTCTCCAACAAAGCCCTCCCAGCCCC |
| | | CATCGAGAAAACCATGTCCAAAGCCAAAGGGCAGCCCCGAGAACCACAGG |
| | | TGTACACCGTGCCCCCATGCCGGGATGAGCTGACCAAGAACCAGGTCAGC |
| | | CTGTGGTGCCTGGTCAAAGGCTTCTATCCCAGCGAGATCGCCGTGGAGTG |
| | | GGAGAGCAATGGGCAGCCGGAGAACAACTACAAGACCACGCCTCCCGTGC |
| | | TGGACTCCGACGGCTCCTTCTTCCTCTACAGCAAGCTCACCGTGGACAAG |
| | | AGCAGGTGGCAGCAGGGGAACGTCTTCTCATGCTCCGTGATGCATGAGGC |
| | | TCTGCACAACCACTACACGCAGAAGAGCCTCTCCCTGTCTCCGGGTAAA |
| 386 | Anti CD1 32 P1A3 | GATGTTGTGATGACTCAGTGTCCACTCTCCCTGCCCGTCACCCCTGGAGA |
| | Fab LC | GCCGGCCTCCATCTCCTGCAGGTCTAGTCAGAGCCTCCTGCATAGTAATG |
| | (VL, joint CL) | GATACAACTATTTGGATTGGTACCTGCAGAAGCCAGGGCAGTCTCCACAG |
| | | CTCCTGATCTATTTGGGTTCTAACCGGGACTCTGGGGTCCCAGACAGATT |
| | | CAGCGGCAGTGGGTCAGGCACTGATTTCAGAGTGAAAATCAGCAGGGTGG |
| | | AGGCTGAGGATGTTGGGGTTTATTACTGCATGCAAGGTACACACTGGCCG |
| | | TGGACGTTCGGCCAAGGGACCAAGGTGGAAATCAAACGAACTGTGGCTGC |
| | | ACCATCTGTCTTCATCTTCCCGCCATCTGATGAGCAGTTGAAATCTGGAA |
| | | CTGCCTCTGTTGTGTGCCTGCTGAATAACTTCTATCCCAGAGAGGCCAAA |
| | | GTACAGTGGAAGGTGGATAACGCCCTCCAATCGGGTAACTCCCAGGAGAG |
| | | TGTCACAGAGCAGGACAGCAAGGACAGCACCTACAGCCTTCAGCAGCACC |
| | | CTGACGCTGAGCAAAGCAGACTACGAGAAACACAAAGTCTACGCCTGCGA |
| | | AGTCACCCATCAGGGCCTGAGCTCGCCCGTCACAAAGAGCTTCAACAGGG |
| | | GAGAGTGT |
| 387 | Anti CD132 P1A3 | CAGGTGCAGCTACAGCAGTGGGGCGCAGGACTGTTGAAGCCTTCGGAGAC |
| | Fab HC | CCTGTCCCTCACCTGCGCTGTCTATGGTGGGTCCTTCAGTGGTTACTACT |
| | (VH, joint CH1) | GGAGCTGGATCCGCCAGCCCCCAGGGAAGGGGCTGGAGTGGATTGGGGAA |
| | | ATCAATCATAGTGGAAGCACCAACTACAACCCGTCCCTCAAGAGTCGAGC |
| | | CACCATATCAGTAGACACGTCCAAGAACCAGTTCTCCCTGAAGCTGAGCT |
| | | CTGTGACCGCCGCGGACACGGCTGTGTATTACTGTGCGACCAGCCCGGGA |
| | | GGCTATTCCGGGGGATACTTGGAGCACTGGGGCCAGGGAACCCTGGTCAC |
| | | CGTCTCAAGCGCCTCCACCAAGGGCCCATCGGTCTTCCCCCTGGCACCCT |
| | | CCTCCAAGAGCACCTCTGGGGGCACAGCGGCCCTGGGCTGCCTGGTCAAG |
| | | GACTACTTCCCCGAACCGGTGACGGTGTCGTGGAACTCAGGCGCCGTGAC |
| | | CAGCGGCGTCCACACCTTCCCGGCTGTCCTACAGTCCTCAGGACTCTACT |
| | | CCCTCAGCAGCGTAGTGACCGTGCCCTCCAGCAGCTTGGGCACCCAGACC |
| | | TACATCTGCAACGTGAATCACAAGCCCAGCAACACCAAGGTGGACAAGAA |
| | | AGTTGAGCCCAAATCTTGT |
| 388 | Anti CD132 P1A3 scFv | CAGGTCCAGCTGCAGCAGTGGGGAGCCGGCGTGCTGAAACCATCTGAAAC |
| | and Fc with | TCTGAGCCTGACTTGCGCTGTCTACGGGGGGTCCTTCAGTGGCTACTATT |
| | hole modification | GGTCATGGATCAGGCAGCCCCCTGGGAAGGGACTGGAGTGGATCGGGGAA |
| | | ATTAACCACTCCGGATCTACAAACTACAATCCCAGTCTGAAATCACGCGC |
| | | CACCATTTCTGTGGACACCAGTAAGAATCAGTTCAGCCTGAAGCTGAGCA |
| | | GCGTGAGAGCCGCTGATACCGCCGTGTACTATTGCGCAACCAGCCCTGGC |
| | | GGATACTCCGGAGGCTATTTTCAGCATTGGGGCCAGGGGACCCTGGTGAC |
| | | AGTCTCTAGTGGGGGAGGAGGGTCTGGAGGAGGAGGAAGTGGAGGAGGAG |
| | | GCTCCGACGTGGTCATGACTCAGAGCCCACTGTCCCTGCCAGTGACCCCC |
| | | GGCGAGCCTGCTAGTATCTCATGTCGATCAAGCCAGTCACTGCTGCACAG |
| | | CAACGGGTACAATTATCTGGATTGGTACTTGCAGAAGCCAGGCCAGTCTC |
| | | CCCAGCTGCTGATCTATGTGGGCTCGAACCGGGACTCTGGGGTGCCTGAT |
| | | AGATTCAGCGGCAGCGGCTCTGGGACTGACTTTACCCTGAAAATTTCCAG |
| | | AGTCGAGGCAGAAGATGTGGGAGTCTACTATTGCATGCAGGGCACTCATT |
| | | GGCCCTGGACCTTCGGACAGGGCACAAAGGTGGAGATCAAGAACAGCGGC |
| | | GCGGGCACCGCGGCCGCGACTCACACATGCCCACCGTGCCCAGCACCTGA |
| | | ACTCCTGGGGGGACCGTCAGTCTTCGTCTTCCCCCCAAAACCCAAGGACA |
| | | CCCTCATGATCTCCCGGACCCCTGAGGTCACATGCGTGGTGGTGGACGTG |
| | | AGCCACGAAGACCCTGAGGTCAAGTTCAACTGGTAGGTGGAGGGCGTGGA |
| | | GGTGCATAATGCCAAGACAAAGCCGCGGGAGGAGCAGTACAACAGCACGT |
| | | ACCGTGTGGTCAGCGTCCTCACCGTCCTGCACCAGGACTGGCTGAATGGC |
| | | AAGGAGTACAAGTGCAAGGTCTCCAACAAAGCCCTCCCAGCCCCCATCGA |
| | | GAAAACCATCTCCAAAGCCAAAGGGCAGCCCCGAGAACCACAGGTGTGCA |
| | | CCCTGCCCCCATCCCGGGATGAGCTGACCAAGAACCAGGTCAGCGTGTCC |
| | | TGCGCCGTCAAAGGCTTCTATCCCAGCGACATCGCCGTGGAGTGGGAGAG |
| | | CAATGGGCAGCCGGAGAACAACTACAAGACCACGCCTCCCGTGCTGGACT |
| | | GCGACGGCTCCTTCTTCCTCGTGAGGAAGCTCACCGTGGACAAGAGCAGG |
| | | TGGCAGCAGGGGAACGTGTTCTCATGCTCCGTGATGCATGAGGCTCTGCA |
| | | CAACCACTACACGCAGAAGAGCCTCTCCCTGTCTCCGGGTAAA |
| 389 | Anti CD132 P2B9 Fab | TCCTATGAGCTGACTCAGCCACCCTCGATGTCAGTGTCCCCAGGACAGAC |
| | LC (VL, joint CL) | GGCCAGGATCACCTGCTCTGGAGATGCATTGCCAAAACAATTTGCTTTTT |
| | | GGTACCAGCAGAAGCCAGGCCAGGCCCCTGTGTTGGTGATTTATAAAGAC |
| | | ACTGAGAGGCCCTCAGGGATCCCTGAGCGATTCTCTGGCTCCAGCTCAGG |
| | | GACAACAGTCACGTTGACCATCACTGGAGTCCAGGGAGAAGATGAGGCTG |
| | | ACTATTACTGTCAATCTCCAGACAGCAGTGGTACCGTCGAAGTGTTCGGG |
| | | GGGGGACCAAGCTGACCGTCCTAGGTCAGCCCAAGGCTGCCCCCTCGGTC |
| | | ACTCTGTTCCCGCCCTCCTCTGAGGAGCTTCAAGCCAACAAGGCCACACT |
| | | GGTGTGTCTCATAAGTGACTTCTACCCGGGAGCCGTGAGAGTGGCCTGGA |
| | | AGGCAGATAGCAGCCCCGTCAAGGGGGGAGTGGAGACCACCACACCCTCC |
| | | AAACAAAGCAACAACAAGTACGCGGCCAGCAGCTACCTGAGCCTGACGCC |
| | | TGAGCAGTGGAAGTCCCACAGAAGCTACAGCTGCCAGGTCACGCATGAAG |
| | | GGAGCACCGTGGAGAAGACAGTGGCCCCTGCAGAATGT |
| 390 | Anti CD1 32 P2B9 | CAGGTGCAGCTGCAGGAGTCGGGCCCAGGACTGGTGAAGCCTTCGGAGAC |
| | Fab HC (VH, Joint CH1) | CCTGTCCCTCACCTGCACTGTCTCTGGTGGCTCCATCAGCAGTAGTAGTT |
| | | ACTACTGGGGCTGGATCCGCCAGCCCCCAGGGAAGGGGCTGGAGTGGATT |
| | | GGGAGTATCTATTATAGTGGGAGCACCTACTACAACCCGTCCCTCAAGAG |
| | | TCGAGTCACCATATCCGTAGACACGTCCAAGAACCAGTTCTCCCTGAAGC |
| | | TGAGCTCTGTGACCGCCGCAGACACGGCTGTGTATTACTGTGCGGGGGAT |
| | | ATTTTGACTGGTTATGCCCTTGACTACTGGGGCCAGGGAACCCTGGTCAC |
| | | CGTCTCAAGCGCCTCCACCAAGGGCCCATCGGTCTTCCCCCTGGCACCCT |
| | | CCTCCAAGAGCACCTCTGGGGGCACAGCGGCCCTGGGCTGCCTGGTCAAG |
| | | GACTACTTCCCCGAACCGGTGACGGTGTCGTGGAACTCAGGCGCCCTGAC |
| | | CAGCGGCGTCCACACCTTCCCGGCTGTCCTACAGTCCTCAGGACTCTACT |
| | | CCCTCAGCAGCGTAGTGACCGTGCCCTCCAGCAGCTTGGGCACCCAGACC |
| | | TACATCTGCAACGTGAATCACAAGCCGACAAGAAAGTTGAGCCCAAATCT |
| | | TGT |
| 391 | Anti CD1 32 P2B9 | CAGGTGCAGCTGCAGGAAAGCGGACCCGGACTGGTGAAGCCATCTGAAAC |
| | scFv and Fc | ACTGAGCCTGACTTGTACCGTGAGCGGCGGAAGCATCAGCTCCTCTAGTT |
| | with hole | ACTATTGGGGATGGATCAGGCAGCCCCCTGGCAAGGGGCTGGAGTGGATC |
| | modification | GGCAGCATCTACTATAGCGGCTCCACATACTATAACCCTAGCCTGAAATC |
| | | CCGCGTGACAATCTCTGTGGAGACTAGTAAGAATCAGTTCTCTCTGAAAC |
| | | TGTCAAGCGTGACCGCCGCTGATACAGCTGTCTACTATTGCGCAGGCGAC |
| | | ATTCTGACCGGGTACGCCCTGGATTATTGGGGACAGGGCACTCTGGTGAC |
| | | CGTCTCCTCTGGAGGAGGAGGCTCAGGAGGAGGAGGGTCCGGAGGCGGGG |
| | | GAAGTTCATACGAACTGACACAGCCACCCTCTATGAGTGTGTCACCAGGG |
| | | CAGACTGCACGAATCACCTGTAGCGGAGACGCCCTGCCCAAGCAGTTCGC |
| | | TTTTTGGTATCAGCAGAAACCTGGCCAGGCTCCAGTGCTGGTCATCTATA |
| | | AGGATACTGAGCGGCCCTCTGGGATTCCTGAAAGATTCAGTGGCAGCAGC |
| | | AGGGGAACCACAGTGACTCTGACCATTACAGGCGTGCAGGGAGAGGACGA |
| | | AGCCGATTACTATTGCCAGTCCCCCGACAGTTCAGGCACCGTGGAGGTCT |
| | | TTGGGGGGGGAACAAAACTGACTGTGCTGAACAGCGGGGGGGGCACCGCG |
| | | GGGGCGACTCACACATGCCCACCGTGCCCAGCACCTGAACTCCTGGGGGG |
| | | ACCGTCAGTCTTCCTCTTCCCCCCAAAACCCAAGGACACCCTCATGATCT |
| | | CCCGGACCCCTGAGGTCACATGCGTGGTGGTGGACGTGAGCCACGAAGAC |
| | | CCTGAGGTCAAGTTCAACTGGTACGTGGACGGCGTGGAGGTGCATAATGC |
| | | CAAGACAAAGCCGCGGGAGGAGCAGTACAACAGCACGTACCGTGTGGTCA |
| | | GCGTCCTCACCGTCCTGCACCAGGACTGGCTGAATGGCAAGGAGTACAAG |
| | | TGCAAGGTCTCCAACAAAGCCCTCCCAGCCCCCATCGAGAAAACCATCTC |
| | | CAAAGCCAAAGGGCAGCCCCGAGAACCACAGGTGTGCACCCTGCCCCCAT |
| | | CCCGGGATGAGCTGACCAAGAACCAGGTCAGCCTGTCCTGCGCCGTCAAA |
| | | GGCTTCTATCCCAGCGACATCGCCGTGGAGTGGGAGAGCAATGGGCAGCC |
| | | GGAGAACAACTACAAGACCACGCCTCCCGTGCTGGACTCCGACGGCTCCT |
| | | TCTTCCTCGTGAGCAAGCTCACCGTGGACAAGAGCAGGTGGCAGCAGGGG |
| | | AACGTCTTCTCATGCTCCGTGATGCATGAGGCTCTGCACAACCACTACAC |
| | | GCAGAAGAGCCTCTCCCTGTCTCCGGGTAAA |
| 392 | Anti CD132 P1A10 | GAAATTGTGGTGACTCAGTCTCCACTCTCCGTGCCCGTTACCCCTGGAGA |
| | Fab LC | GCCGGGCTCCATCTCCTGCAGGTCTAGTCAGAGCCTCCTGCATAGTAATG |
| | (VL, joint CL) | GATACAACTATTTGAATTGGTACCTACAGAAGCCAGGGCAGTCTCCACAA |
| | | CTCCTGATCTATTTGGGTTCTGATCGGGCCTCCGGGGTCCCTGACAGGTT |
| | | CAGTGGCAGTGGATCAGGCACAGATTTTACACTGAAAATCAGCAGAGTGG |
| | | AGGCTGAGGATGTTGGGGTTTATTACTGCATGCAAGCTCTACAAACCCCC |
| | | ACCACTTTCGGGGGAGGGACCAAGGTGGAGATCAAACGAACTGTGGCTGC |
| | | ACCATCTGTCTTCATCTTCCCGCCATCTGATGAGCAGTTGAAATCTGGAA |
| | | CTGCCTCTGTTGTGTGCCTGCTGAATAACTTCTATCCCAGAGAGGCCAAA |
| | | GTACAGTGGAAGGTGGATAACGCCCTCCAATCGGGTAACTCCCAGGAGAG |
| | | TGTCACAGAGCAGGACAGCAAGGACAGCACCTACAGCCTCAGCAGCACCC |
| | | TGACGCTGAGCAAAGCAGACTACGAGAAACACAAAGTCTACGCCTGCGAA |
| | | GTCACCCATCAGGGCCTGAGCTCGCCCGTCACAAAGAGCTTCAACAGGGG |
| | | AGAGTGT |
| 393 | Anti CD132 P1A10 | CAGGTACAGCTGCAGCAGTCAGGGGCTGAGGTGAAGAAGCCTGGGTCCTC |
| | Fab HC | GGTGAAGGTCTCCTGCAAGGCTTCTGGAGGCACCTTCAGCAGCTATGCTA |
| | (VH, joint CH1) | TCAGCTGGGTGCGACAGGCCCCTGGACAAGGGCTTGAGTGGATGGGAGGT |
| | | TTTGATCCTGAAGATGGTGAAACAATCTACGCACAGAAGTTCCAGGGCAG |
| | | AGTCACCATGACCGAGGACACATCTACAGACACAGCCTACATGGAGCTGA |
| | | GCAGCCTGAGATCTGAGGACACGGCCGTGTATTAGTGTGCAACAGATCTG |
| | | AGAATTCCGTATTACTATGATAACCCCTGGGGCCAGGGCACCCTGGTCAC |
| | | CGTCTCAAGCGCCTCCACCAAGGGCCCATCGGTCTTCCCCCTGGCACCCT |
| | | CCTCCAAGAGCACCTCTGGGGGCACAGCGGCCCTGGGCTGCCTGGTCAAG |
| | | GACTACTTCCCCGAACCGGTGACGGTGTCGTGGAACTCAGGCGCCCTGAC |
| | | CAGCGGCGTCCACACCTTCCCGGCTGTCCTACAGTCCTCAGGACTCTACT |
| | | CCCTCAGCAGCGTAGTGACCGTGCCCTCCAGCAGCTTGGGCACCCAGACC |
| | | TACATCTGCAACGTGAATCACAAGCCCAGCAACACCAAGGTGGACAAGAA |
| | | AGTTGAGCCCAAATCTTGT |
| 394 | Anti CD132 | CAGGTCCAGCTGCAGCAGAGGGGAGCCGAGGTCAAGAAGCCAGGGAGTAG |
| | P1A10 scFv | CGTCAAAGTCAGTTGTAAAGCATCAGGAGGAACATTCAGCTCCTATGCAA |
| | and Fc with hole | TCTCTTGGGTGCGACAGGCCCCTGGACAGGGCCTGGAGTGGATGGGAGGA |
| | modification | TTCGACCCAGAGGATGGAGAAACCATCTACGCCCAGAAGTTTCAGGGCAG |
| | | AGTGACTATGACCGAAGACACATCTACTGATACCGCTTACATGGAGCTGT |
| | | CTAGTCTGAGGAGTGAAGACACTGCCGTCTACTATTGCGCTACCGACCTG |
| | | CGCATCCCATACTATTACGATAATCCGTGGGGGCAGGGAACACTGGTGAC |
| | | TGTCTCAAGCGGAGGGGGGGGATCAGGGGGAGGAGGCAGCGGAGGAGGAG |
| | | GGTCCGAGATCGTGCTGACACAGAGTCCACTGTCACTGCCAGTCACCCCT |
| | | GGCGAACCAGCCAGTATTTCATGTCGGTCCTCTCAGAGCCTGCTGCACTC |
| | | CAACGGGTATAATTACCTGAACTGGTACTTGCAGAAGCCTGGCCAGAGCC |
| | | CTCAGCTGCTGATCTACCTGGGCTCTGACCGAGCAAGTGGGGTGCCCGAT |
| | | AGATTCAGCGGCTCCGGGTCTGGAACCGACTTTACCCTGAAGATCAGCCG |
| | | GGTGGAGGCTGAAGATGTGGGCGTCTATTACTGCATGCAGGCCCTCCAGA |
| | | CACCTACCACATTCGGAGGGGGGACTAAGGTGGAGATCAAGAACAGCGGC |
| | | GCGGGCACCGCGGCCGCGACTCACACATGCCCACCGTGCCCAGCACCTGA |
| | | ACTCCTGGGGGGACCGTCAGTCTTCCTCTTCCCCCCAAAACCCAAGGACA |
| | | CCCTCATGATCTCCCGGACCCCTGAGGTCACATGCGTGGTGGTGGACGTG |
| | | AGCCACGAAGACCCTGAGGTCAAGTTCAACTGGTACGTGGACGGCGTGGA |
| | | GGTGCATAATGCCAAGACAAAGCCGCGGGAGGAGCAGTACAACAGCACGT |
| | | ACCGTGTGGTCAGCGTCCTCACCGTCCTGCACCAGGACTGGCTGAATGGC |
| | | AAGGAGTACAAGTGCAAGGTCTCCAACAAAGCCCTCCCAGCCCCCATCGA |
| | | GAAAACCATCTCCAAAGCCAAAGGGCAGCCCCGAGAACCACAGGTGTGCA |
| | | CCCTGCCCCCATCCCGGGATGAGCTGACCAAGAACCAGGTCAGCCTGTCC |
| | | TGCGCCGTCAAAGGCTTCTATCCCAGCGACATCGCCGTGGAGTGGGAGAG |
| | | CAATGGGCAGCCGGAGAACAACTACAAGACCACGCCTCCCGTGCTGGACT |
| | | CCGACGGCTCCTTCTTCCTCGTGAGCAAGCTCACCGTGGACAAGAGCAGG |
| | | TGGCAGCAGGGGAACGTCTTCTCATGCTCCGTGATGCATGAGGCTCTGCA |
| | | CAACCACTACACGCAGAAGAGCCTCTCCCTGTCTCCGGGTAAA |
| 395 | Anti CD132 P1B6 | GAAATTGTGATGACGCAGTCTCCACTCTCCCTGCCCGTCACCCCTGGAGA |
| | Fab LC | GCCGGCCTCCATCTCCTGCAGGTCTAGTCAGAGCCTCCTGCATAGTAATG |
| | (VL, joint CL) | GATACAACTATTTGGATTGGTACCTGCAGAAGCCAGGGCAGTCTCCACAG |
| | | CTCCTGATGTATTTGGTTTCTAATCGGGCCTCCGGGGTCCCTGAGAGGTT |
| | | CAGTGGCAGTGGATCAGGCACAGATTTTACACTGAAAATCAGCAGAGTGG |
| | | AGGCTGAGGATGTTGGGGTTTATTACTGCATGCAAACTCTACAAACTCCT |
| | | CTCAGTTTTGGCCAGGGGACCAAGCTGGAGATCAAACGAACTGTGGCTGC |
| | | ACCATCTGTCTTCATCTTCCCGCCATCTGATGAGCAGTTGAAATCTGGAA |
| | | CTGCCTCTGTTGTGTGGCTGGTGAATAACTTCTATCCCAGAGAGGCCAAA |
| | | GTACAGTGGAAGGTGGATAACGCCCTCCAATGGGGTAACTCCCAGGAGAG |
| | | TGTCACAGAGCAGGACAGCAAGGACAGCACCTACAGCCTCAGCAGCACCC |
| | | TGACGCTGAGCAAAGCAGACTACGAGAAACACAAAGTCTACGCCTGCGAA |
| | | GTCACCCATCAGGGCCIGAGCTCGCCCGTCACAAAGAGGTTCAACAGGGG |
| | | AGAGTGT |
| 396 | Anti CD132 | CAGGTCCAGCTGGTACAGTCTGGGGGAGGCGTGGTCCAGCCTGGGAGGTC |
| | P1B6 Fab | CCTGAGACTCTCCTGTGCAGCCTCTGGATTCACCTTCAGTAGCTATGCTA |
| | HC | TGCACTGGGTCCGCCAGGCTCCAGGCAAGGGGCTGGAGTGGGTGGCAGTT |
| | (VH, joint CH1) | ATATCATATGATGGAAGCAATAAATACTACGCAGACTCCGTGAAGGGCCG |
| | | ATTCACCATCTCCAGAGACAATTCCAAGAACACGCTGTATCTGCAAATGA |
| | | ACAGCCTGAGAGCCGAGGACACGGCTGTGTATTACTGTGCGAGAAGTCTT |
| | | TACTACAGCCACTTTGACTACTGGGGCCAGGGAACCCTGGTCACCGTCTC |
| | | AAGCGCCTCCACCAAGGGCCCATCGGTCTTCCCCCTGGCACCCTCCTCCA |
| | | AGAGCACCTCTGGGGGCACAGCGGCCCTGGGCTGCCTGGTCAAGGACTAC |
| | | TTCCCCGAACCGGTGACGGTGTCGTGGAACTCAGGCGCCCTGACCAGCGG |
| | | CGTCCACACCTTCCCGGCTGTCCTACAGTCCTCAGGACTCTACTCCCTCA |
| | | GCAGCGTAGTGACCGTGCCCTCCAGCAGCTTGGGCACCCAGACCTACATC |
| | | TGCAACGTGAATCACAAGCCCAGCAACACCAAGGTGGACAAGAAAGTTGA |
| | | GCCCAAATCTTGT |
| 397 | Anti CD132 P1B6 | CAGGTGCAGCTGGTCCAGAGCGGAGGAGGCGTCGTCCAGCCCGGAAGGTC |
| | scFv and | ACTGAGACTGTCTTGTGCCGCATCAGGATTCACTTTTAGCTCCTACGCAA |
| | Fc with hole | TGCACTGGGTGAGGCAGGCCCCTGGCAAGGGGCTGGAGTGGGTGGCTGTC |
| | modification | ATCAGTTATGACGGCTCAAACAAGTACTATGCAGATAGCGTGAAAGGGGG |
| | | GTTCACCATTAGCAGAGACAACTCCAAAAATACACTGTACCTCCAGATGA |
| | | ACAGCCTGCGAGCCGAAGACACAGCTGTGTACTATTGCGCCCGGTCTCTG |
| | | TACTATAGTCACTTTGATTACTGGGGACAGGGCACCCTGGTGACAGTCTC |
| | | TAGTGGCGGGGGAGGCAGTGGAGGAGGAGGGAGCGGAGGAGGAGGCAGCG |
| | | AGATCGTGATGACTCAGTCCCCACTGTGTCTGCCAGTCACCCCTGGGGAA |
| | | CCAGCATCCATTTCTTGTAGATCAAGCCAGTCACTGCTGCATAGCAACGG |
| | | ATACAATTATCTGGATTGGTACTTGCAGAAGCCTGGGGAGTCTCGTCAGC |
| | | TGCTGATGTATCTGGTGTCCAACAGGGCCTCTGGGGTCCCAGAGCGCTTC |
| | | AGTGGGTCAGGAAGCGGCACTGACTTTACCCTGAAAATCTCTCGCGTGGA |
| | | GGCTGAAGATGTGGGCGTCTACTATTGCATGCAGACACTCCAGACTCCCC |
| | | TGAGCTTCGGGCAGGGAACCAAGCTGGAGATCAAGAACAGCGGCGCGGGC |
| | | ACCGCGGCCGCGACTCACACATGCCCACCGTGCCCAGCACCTGAACTCCT |
| | | GGGGGGACCGTCAGTCTTCCTCTTCCCCCCAAAACCCAAGGACACCCTCA |
| | | TGATCTCCCGGACCCCTGAGGTCACATGCGTGGTGGTGGACGTGAGCCAC |
| | | GAAGACCCTGAGGTCAAGTTCAACTGGTACGTGGACGGCGTGGAGGTGCA |
| | | TAATGCCAAGACAAAGCCGCGGGAGGAGCAGTACAACAGCACGTACCGTG |
| | | TGGTCAGCGTCCTCACCGTCCTGCACCAGGACTGGCTGAATGGCAAGGAG |
| | | TACAAGTGCAAGGTCTCCAACAAAGCCCTCCCAGCCCCCATCGAGAAAAC |
| | | CATCTCCAAAGCCAAAGGGGAGCCCCGAGAACCACAGGTGTGCACCCTGC |
| | | CCCCATCCCGGGATGAGCTGACCAAGAACCAGGTCAGCCTGTCCTGCGCC |
| | | GTCAAAGGCTTCTATCCCAGCGACATCGCCGTGGAGTGGGAGAGCAATGG |
| | | GCAGCCGGAGAACAACTACAAGACCACGCCTCCCGTGCTGGACTCCGACG |
| | | GCTCCTTCTTCCTCGTGAGCAAGCTCACCGTGGACAAGAGCAGGTGGCAG |
| | | CAGGGGAACGTCTTCTCATGCTCCGTGATGCATGAGGCTCTGCACAACCA |
| | | CTACACGCAGAAGAGCCTCTCCCTGTCTCCGGGTAAA |
| 398 | | GAAATTGTGCTGACTCAGTCTCCAGCCACCCTGTCTTTGTCTCCAGGGGA |
| | Anti CD132 | ACGAGCCACCCTCTCCTGCAGGGCCAGTCAGAGTGTTAGTTACCACTTAG |
| | P1C10 Fab | CCTGGTACCAACAAAAACCTGGCCAGGCTCCCAGGCTCCTCATCTATGAT |
| | LC | ACATCCAACAGGGCCTCTGGCATCCCCGCCAGGTTCAGTGGCAGTGGGTC |
| | (VL, joint CL) | TGGGACAGACTTCACTCTCACCATCAACAGCCTAGAGCCTGAAGATTTTG |
| | | CAGTTTATTACTGTCAGCAGCGTTACGACTGGCCTCTCACTTTCGGCGGA |
| | | GGGACCAAGGTGGAGATCAAACGAACTGTGGCTGCACCATCTGTCTTCAT |
| | | CTTCCCGCCATCTGATGAGCAGTTGAAATCTGGAACTGCCTCTGTTGTGT |
| | | GCCTGCTGAATAACTTCTATCCCAGAGAGGCCAAAGTACAGTGGAAGGTG |
| | | GATAACGCCCTCCAATCGGGTAACTCCCAGGAGAGTGTCACAGAGCAGGA |
| | | CAGCAAGGACAGCACCTACAGCCTCAGCAGCACCCTGACGCTGAGCAAAG |
| | | CAGACTACGAGAAACACAAAGTCTACGCCTGCGAAGTCACCCATCAGGGC |
| | | CTGAGCTCGCCCGTCACAAAGAGCTTCAACAGGGGAGAGTGT |
| 399 | Anti CD132 | GAGGTGCAGCTGGTGGAGACTGGCCCAGGACTGGTGAAGCCTTCGGGGAC |
| | P1C10 Fab | CCTGTCCCTCACCTGCGCTGTCTCTGGTGGCTCCATCAGCAGTAGTAACT |
| | HC | GGTGGAGTTGGGTCCGCCAGCCCCCAGGGAAGGGGCTGGAGTGGATTGGG |
| | (VH, joint CH1) | GAAATCTATCATAGTGGGAGCACCAACTACAACCCGTCGCTCAAGAGTCG |
| | | AGTCACCATATCAGTAGAGAAGTCCAAGAACCAGTTCTCCCTGAAGGTGA |
| | | GCTCTGTGACCGCCGCGGACACGGCCGTGTATTAGTGTGCGAGAGAAGGG |
| | | CCCCTAAGGAGCAGCGGACCGGGTGCTTTTGATATCTGGGGCCAAGGGAC |
| | | AATGGTCACCGTCTCAAGCGCCTCCACCAAGGGCCCATCGGTCTTCCCCC |
| | | TGGCACCCTCCTCCAAGAGCACCTCTGGGGGCACAGCGGCCCTGGGCTGC |
| | | CTGGTCAAGGACTACTTCCCCGAACCGGTGACGGTGTCGTGGAACTCAGG |
| | | CGCCCTGACCAGCGGCGTCCACACCTTCCCGGCTGTCCTACAGTCCTCAG |
| | | GACTCTACTCCCTCAGCAGCGTAGTGACCGTGCCCTCCAGCAGCTTGGGC |
| | | ACCCAGACCTACATCTGCAACGTGAATCACAAGCCCAGCAACACCAAGGT |
| | | GGACAAGAAAGTTGAGCCCAAATCTTGT |
| 400 | Anti CD132 | CAGGTCCAGCTGCAGGAATCAGGAGGGGGGGTCGTCCAGCCAGGGAGGTC |
| | P1C10 scFv | ACTGAGACTGTCTTGCGCCGCTTCAGGGTTCACTTTTAGCAACTACGGAA |
| | and Fc with hole | TGCACTGGGTGCGGCAGGCTCCCGGGAAAGGGCTGGAGTGGGTGGCAGTC |
| | modification | ATCTCTTATGAGGGCACAAACAAGTACTATGCAGATAGTGTCAAGGGGCG |
| | | GTTCACCATCAGCCGGGACAACAGTAAAAATACAGTGTACCTCCAGATGA |
| | | ACAGCCTGCGGGCCGAAGATACTGCTGTCTACTATTGCGCCAAGGAGGGG |
| | | TTTGACATCTGGGGACAGGGCACTATGGTGACCGTCAGCTCGGGGGGGGG |
| | | AGGGTCAGGAGGAGGAGGGAGCGGAGGAGGAGGGAGCGACATTCAGATGA |
| | | CCCAGTCACCTAGCTTCCTGTCCGCTTCTGTGGGCGATAGGGTCACAATC |
| | | ACTTGTCGCGCCAGTCAGTCAATTTCTAGTTGGCTGGCTTGGTATCAGCA |
| | | GAAGCCCGGAAAAGCACCTAAGCTGCTGATCTATGAGGCCTCCCGACTGG |
| | | AGGATGGCGTGCCAAGCAGATTCTCCGGGACAGGATTTGGCACTGACTTC |
| | | ACCTTTACAATCACCACACTCCAGCCAGACGATATTGCCACTTACTATTG |
| | | CCAGCAGTACGACGATCTGCCCTATACCTTTGGGCAGGGAACTACCGTGG |
| | | ATATTAAGAAGAGGGGCGGGGGCACCGGGGCCGCGACTCACACATGCCCA |
| | | CCGTGCCCAGCACCTGAACTCCTGGGGGGACCGTCAGTCTTCCTCTTCCC |
| | | CCCAAAACCCAAGGACACCCTCATGATCTCCCGGACCCCTGAGGTCACAT |
| | | GCGTGGTGGTGGACGTGAGCCACGAAGACCCTGAGGTCAAGTTCAACTGG |
| | | TACGTGGACGGCGTGGAGGTGCATAATGCCAAGACAAAGCCGCGGGAGGA |
| | | SCAGTACAACAGCACGTACCGTGTGGTCAGCGTCCTCACCGTCCTGCACC |
| | | AGGACTGGCTGAATGGCAAGGAGTACAAGTGCAAGGTCTGCAACAAAGCC |
| | | CTCCCAGCCCCCATCGAGAAAACCATCTCCAAAGCCAAAGGGCAGCCCCG |
| | | AGAACCACAGGTGTGCACCCTGCCCCCATCCCGGGATGAGCTGACCAAGA |
| | | ACCAGGTCAGCCTGTCCTGCGCGGTCAAAGGCTTCTATCCCAGCGACATC |
| | | GCCGTGGAGTGGGAGAGCAATGGGCAGCCGGAGAACAACTACAAGACCAC |
| | | GCCTGCCGTGCTGGACTCCGACGGCTCGTTCTTCCTGGTGAGCAAGCTCA |
| | | CCGTGGACAAGAGCAGGTGGCAGCAGGGGAACGTCTTCTCATGCTCCGTG |
| | | ATGCATGAGGCTCTGCACAACCACTACACGCAGAAGAGCCTCTCCCTGTC |
| | | TCCGGGTAAA |
| 401 | Anti CD132 P1D7 | GACATCCAGATGACCCAGTCTCCTTCCTTCCTGTCTGCATCTGTAGGAGA |
| | Fab LC | CAGAGTCACCATCACTTGCCGGGCCAGTCAGAGTATTAGTAGCTGGTTGG |
| | (VL joint CL) | CCTGGTATCAGCAGAAACCAGGGAAAGCCCCTAAACTCCTGATCTACGAT |
| | | GCATCCCGTTTGGAGGACGGGGTCCCATCAAGATTCAGTGGAACTGGATT |
| | | TGGGACAGATTTTACTTTCACCATTACCACCCTGCAGCCTGACGATATTG |
| | | CGAGATATTATTGTCAGCAATACGATGATCTGCCGTACACTTTTGGCCAG |
| | | GGGACCACGGTGGACATCAAACGAACTGTGGCTGCACCATCTGTCTTCAT |
| | | CTTCCCGCCATCTGATGAGCAGTTGAAATGTGGAACTGCCTCTGTTGTGT |
| | | GCCTGCTGAATAACTTCTATCCCAGAGAGGCCAAAGTACAGTGGAAGGTG |
| | | GATAACGCCCTCCAATCGGGTAACTCCCAGGAGAGTGTCACAGAGCAGGA |
| | | CAGCAAGGACAGCACCTACAGCCTCAGCAGCACCCTGACGCTGAGCAAAG |
| | | CAGACTACGAGAAACACAAAGTCTACGCCTGCGAAGTCACCCATCAGGGG |
| | | CTGAGCTCGCCCGTCACAAAGAGCTTCAACAGGGGAGAGTGT |
| 402 | Anti CD132 P1D7 | CAGGTGCAGCTGCAGGAGTCCGGGGGAGGCGTGGTCCAGCCTGGGAGGTC |
| | Fab HC | CCTGAGACTCTCCTGTGCAGCCTCTGGATTCACCTTCAGTAACTATGGCA |
| | (VH, joint CH1) | TGCACTGGGTCCGCCAGGCTCCAGGCAAAGGGCTGGAGTGGGTGGCAGTT |
| | | ATATCATATGATGGAACTAATAAATACTATGCAGACTCCGTGAAGGGCCG |
| | | ATTCACCATCTCCAGAGACAATTCCAAGAACACGGTGTATCTGCAAATGA |
| | | ACAGGCTGAGAGCTGAGGACACGGCTGTGTATTACTGTGCGAAAGATGGT |
| | | TTTGATATTTGGGGCCAAGGGACAATGGTCACCGTCTCAAGCGCCTCCAC |
| | | CAAGGGCCCATCGGTCTTCCCCCTGGCACCCTCCTCCAAGAGCACCTCTG |
| | | GGGGCACAGCGGCCCTGGGCTGCCTGGTCAAGGACTACTTCCCCGAACCG |
| | | GTGACGGTGTCGTGGAACTCAGGCGCCCTGACCAGCGGCGTCCACACCTT |
| | | CCCGGCTGTCCTACAGTCCTCAGGACTCTACTCCCTCAGCAGCGTAGTGA |
| | | CCGTGCCCTCCAGCAGCTTGGGCACCCAGACCTACATCTGCAACGTGAAT |
| | | CAGAAGCCCAGCAACACCAAGGTGGACAAGAAAGTTGAGCCCAAATCTTG |
| | | T |
| 403 | Anti CD132 | GAAGTGCAGCTGGTGGAAACTGGACCTGGACTGGTGAAGCCAAGGGGACT |
| | P107 scFv | CTGAGCCTGACCTGTGCCGTGAGCGGGGGAAGTATCAGCTCCTCTAACTG |
| | and Fc | GTGGTCCTGGGTGCGACAGCCCCCTGGCAAGGGGCTGGAGTGGATGGGCG |
| | with hole | AAATCTACCAGAGCGGGTCCACAAACTATAATCCTAGCCTGAAGAGCCGG |
| | modification | GTGACTATCTCTGTGGACAAGAGTAAAAATCAGTTCAGCCTGAAACTGAG |
| | | TTCAGTGACAGCCGCTGATACCGCCGTGTACTATTGCGCCAGGGAGGGAC |
| | | CTCTGAGCAGCAGCGGACCAGGCGCTTTTGACATCTGGGGGCAGGGAACT |
| | | ATGGTGACCGTCAGTTCAGGGGGAGGAGGCTCCGGAGGAGGAGGGTCTGG |
| | | AGGGGGGGGAAGTGAGATTGTGCTGAGCCAGTCCCCCGCCACACTGTCTC |
| | | TGAGTCCTGGCGAACGGGCCACCCTGTCTTGTAGAGCTTCACAGAGCGTG |
| | | TCCTACCATCTGGCATGGTATCAGCAGAAACCAGGCCAGGCCCCCAGACT |
| | | GCTGATCTACGACACCTCAAACAGGGCTAGCGGCATTCCCGCACGCTTCT |
| | | CTGGCAGTGGGTCAGGAACAGATTTTACCCTGACAATCAATAGCCTGGAG |
| | | CCAGAAGACTTCGCCGTGTACTATTGCCAGCAGCGCTATGATTGGCCCCT |
| | | GACTTTTGGGGGGGGAACCAAGGTCGAGATCAAGAACAGCGGCGCGGGCA |
| | | CCGCGGCCGCGACTCACACATGCCCACCGTGCCCAGCACCTGAACTCCTG |
| | | GGGGGACCGTCAGTCTTCCTCTTCCCCCCAAAACCCAAGGACACCCTCAT |
| | | GATCTCCCGGACCCCTGAGGTCACATGCGTGGTGGTGGACGTGAGCCACG |
| | | AAGACCCTGAGGTCAAGTTCAACTGGTACGTGGACGGCGTGGAGGTGCAT |
| | | AATGCCAAGACAAAGCCGCGGGAGGAGCAGTACAACAGCACGTACCGTGT |
| | | GGTCAGCGTCCTCACCGTCCTGCACCAGGACTGGCTGAATGGCAAGGAGT |
| | | ACAAGTGCAAGGTCTCCAACAAAGCCCTCCCAGCCCCCATCGAGAAAACC |
| | | ATCTCCAAAGCCAAAGGGCAGCCCCGAGAACCACAGGTGTGCACCCTGCC |
| | | CCCATCCCGGGATGAGCTGACCAAGAACCAGGTCAGCCTGTCCTGCGCCG |
| | | TCAAAGGCTTCTATCCCAGCGACATCGCCGTGGAGTGGGAGAGCAATGGG |
| | | CAGCCGGAGAACAACTACAAGACCACGCCTCCCGTGCTGGACTCCGACGG |
| | | CTCCTTCTTCCTCGTGAGCAAGCTCACCGTGGACAAGAGCAGGTGGCAGC |
| | | AGGGGAACGTCTTCTCATGCTCCGTGATGCATGAGGCTCTGCACAACCAC |
| | | TACACGCAGAAGAGCCTCTCCCTGTCTCCGGGTAAA |
| 404 | Anti CD132 P1E8 | GATGTTGTGATGACTCAGTCTCCAGTGTCCCTGCCCGTCACCCTTGGACA |
| | Fab LC | GCCGGCCTCCATCTCCTGCAAGTCTAGTCAAAGCCTCCTTTACTTTAATG |
| | (VL, joint CL) | GAAACACCTACTTGAGCTGGTTTCAGCAGAGGCCAGGCCAATCTCCACGG |
| | | CGCCTATTTTATCAGGTTTCTAACCGGGACTCTGGGGTCCCAGACAGATT |
| | | CAGCGGCAGTGGGTCAGACACTGATTTCACTCTGACCATTAGCAGGGGGA |
| | | GGCTGAAGATGTTGGAGTTTATTTCTGCATGCAAGGAACACAGTGGCCTC |
| | | CGACGTTCGGCCAAGGGACCAAGGTGGAAATCAAACGAACTGTGGCTGCA |
| | | CCATCTGTCTTCATCTTCCCGCCATCTGATGAGCAGCTGAAATCTGGAAC |
| | | TGCCTCTGTTGTGTGCCTGCTGAATAACTTCTATCCCAGAGAGGCCAAAG |
| | | TACAGTGGAAGGTGGATAACGCCCTCCAATCGGGTAACTCCCAGGAGAGT |
| | | GTCACAGAGCAGGACAGCAAGGACAGCACCTACAGCCTCAGCAGCACCCT |
| | | GACGCTGAGCAAAGCAGACTACGAGAAACACAAAGTCTACGCCTGCGAAG |
| | | TCACCCATCAGGGCCTGAGCTCGCCCGTCACAAAGAGCTTCAACAGGGGA |
| | | GAGTGT |
| 405 | Anti CD132 PHE8 | GAGGTCCAGCTGGTGCAGTGTGGGGGAGGCGTGGTCCAGCCTGGGAGGTC |
| | Fab HC | CCTGAGACTCTCCTGTGCAGCCTCTGGATTCACCTTCAGTAGCTATGGCA |
| | (VH, joint CH1) | TGCACTGGGTCCGCCAGGCTCCAGGCAAGGGGCTGGAGTGGGTGGCAGTT |
| | | ATATCATATGATGGAAGTAATAAATACTATGCAGACTCCGTGAAGGGCCG |
| | | ATTCACCATCTCCAGAGACAATTCCAAGAACACGCTGTATCTGCAAATGA |
| | | ACAGCCTGAGAGCCGAGGACACGGCCGTGTATTACTGTGCGAGAGATGTC |
| | | TACGGTGACTACGGGGCCTTTGACTACTGGGGCCAGGGAACCCTGGTCAC |
| | | CGTCTCAAGCGCCTCCACCAAGGGCCCATCGGTCTTCCCCCTGGCACCCT |
| | | CCTCCAAGAGCACCTCTGGGGGCACAGCGGCCCTGGGCTGCCTGGTCAAG |
| | | GACTACTTCCCCGAACCGGTGACGGTGTCGTGGAACTCAGGCGCCCTGAC |
| | | CAGCGGCGTCCACACCTTCCCGGCTGTCCTACAGTCCTCAGGACTCTACT |
| | | CCCTCAGCAGCGTAGTGACCGTGCCCTGCAGCAGCTTGGGCAGCCAGACC |
| | | TACATCTGCAACGTGAATCACAAGCCCAGCAACACCAAGGTGGACAAGAA |
| | | AGTTGAGCCCAAATCTTGT |
| 406 | Anti CD132 | GAGGTCCAGCTGGTCCAGAGCGGCGGAGGGGTCGTCCAGGGGGGAAGAAG |
| | P1E8 scFv and | CCTGAGACTGTCCTGTGCAGCAAGTGGGTTTACATTCAGCTCCTACGGCA |
| | Fc with hole | TGCACTGGGTGAGGCAGGCACCCGGCAAGGGGCTGGAGTGGGTGGCCGTC |
| | modification | ATCAGTTATGACGGCTCAAACAAGTACTATGCCGATAGCGTGAAAGGGAG |
| | | GTTCACAATTAGCCGCGACAACTCCAAAAATACTCTGTACCTCCAGATGA |
| | | ACAGCCTGAGAGCCGAAGATACAGCTGTGTACTATTGCGCTAGGGACGTC |
| | | TACGGAGATTATGGCGCATTTGACTATTGGGGACAGGGCACTCTGGTGAC |
| | | CGTCTCTAGTGGAGGAGGAGGCTCAGGAGGAGGAGGGAGCGGCGGAGGAG |
| | | GCAGCGATGTGGTCATGACCCAGTGCCGAGTGTCTCTGGCAGTCACACTG |
| | | GGACAGCCAGCATCCATCTCTTGTAAGTGAAGCCAGTCTCTGCTGTACTT |
| | | CAAGGGAAATACTTATCTGTCTTGGTTTCAGCAGCGCCCTGGCCAGAGTC |
| | | CACGGAGACTGTTCTACCAGGTGTCTAACCGAGACAGTGGCGTCCCTGAT |
| | | CGGTTCAGTGGGTCAGGAAGCGACACCGATTTTACCCTGACAATCAGCCG |
| | | AGTGGAGGCTGAAGACGTGGGGGTCTATTTCTGCATGCAGGGAACACAGT |
| | | GGCCCCCTACTTTTGGCCAGGGGACCAAGGTGGAGATCAAGAACAGCGGG |
| | | GGGGGCACCGGGGCCGCGACTCACACATGCCCACCGTGCCCAGCACCTGA |
| | | ACTCGTGGGGGGACCGTCAGTCTTCCTCTTCCCCCCAAAACCCAAGGACA |
| | | CCCTCATGATCTCCCGGACCCCTGAGGTCACATGCGTGGTGGTGGACGTG |
| | | AGCCACGAAGACCCTGAGGTCAAGTTCAACTGGTAGGTGGACGGCGTGGA |
| | | GGTGCATAATGCCAAGACAAAGCCGCGGGAGGAGCAGTACAACAGCACGT |
| | | ACCGTGTGGTCAGCGTCCTCACCGTCCTGCACCAGGACTGGCTGAATGGC |
| | | AAGGAGTACAAGTGCAAGGTCTCCAACAAAGCCCTCCCAGCCCCCATCGA |
| | | GAAAACCATCTCCAAAGCCAAAGGGCAGCCCCGAGAACCACAGGTGTGCA |
| | | CCCTGCCCCCATCCCGGGATGAGCTGACCAAGAACCAGGTCAGCCTGTCC |
| | | TGCGCCGTCAAAGGCTTCTATCCCAGCGACATCGCCGTGGAGTGGGAGAG |
| | | CAATGGGCAGCCGGAGAACAACTACAAGACCACGCCTCCCGTGCTGGACT |
| | | CCGACGGCTCCTTCTTCCTCGTGAGCAAGCTCACCGTGGACAAGAGCAGG |
| | | TGGCAGCAGGGGAACGTCTTCTCATGCTCCGTGATGCATGAGGCTCTGCA |
| | | CAACCACTACACGCAGAAGAGCCTCTCCCTGTCTCCGGGTAAA |
| 407 | Anti CD132 P2B2 | GATGTTGTGATGAGTCAGTCTCCACTCTCGCTGCCCGTCACCCCTGGAGA |
| | Fab LC | GCCGGCCTCCATCTCCTGCAGGTCTAGTCAGAGCCTCCTGCATAGTAATG |
| | (VL, joint CL) | GATACAACTATTTGGATTGGTACCTGCAGAAGCCAGGGCAGTCTCCACAC |
| | | CTCCTGATCTACTTGGGTTCTAATCGGGCCTCCGGGGTCCCTGACAGGTT |
| | | CAGTGGCAGTGGATCAGGCACAGATTTTACACTGAAAATTAGCAGAGTGG |
| | | AGGCTGAGGATGTTGGGGTTTATTTCTGCATGCAAGCTCTACGAACTCCG |
| | | TACACTTTTGGCCAGGGGACCAAGCTGGAGATCAAACGAACTGTGGCTGC |
| | | ACCATCTGTCTTCATCTTCCCGCCATCTGATGAGCAGTTGAAATCTGGAA |
| | | CTGCCTCTGTTGTGTGCCTGCTGAATAACTTCTATCCCAGAGAGGCCAAA |
| | | GTACAGTGGAAGGTGGATAACGCCCTCCAATCGGGTAACTCCCAGGAGAG |
| | | TGTCACAGAGCAGGACAGCAAGGACAGCACCTACAGCCTCAGCAGCACCC |
| | | TGACGCTGAGCAAAGCAGACTACGAGAAACACAAAGTCTACGCCTGCGAA |
| | | GTCACCCATCAGGGCCTGAGCTCGCCCGTCACAAAGAGCTTCAACAGGGG |
| | | AGAGTGT |
| 408 | Anti CD132 | CAGCTGCAGCTGCAGGAGTCGGGGGGAGGCGTGGTCCAGCCTGGGAGGTC |
| | P2B2 Fab | CCTGAGACTCTCCTGTGCAGCCTCTGGATTCACCTTCAGTAGCTATGCTA |
| | HC | TGCACTGGGTCCGCCAGGCTCCAGGCAAGGGGCTGGAGTGGGTGGCAGTT |
| | (VH, joint CH1) | ATATCATATGATGGAGGTAATAAATACTACGCAGACTCCGTGAAGGGCCG |
| | | ATTCACCATCTCCAGAGACAATTCCAAGAACACGCTGTATCTGCAAATGA |
| | | ACAGCCTGAGAGGTGAGGACACGGCTGTGTATTACTGTGCGAAATCAGTG |
| | | GCGCCTCCCATGGACGTCTGGGGCAAAGGGACCACGGTCACCGTCTCAAG |
| | | CGCCTCCACCAAGGGCCCATCGGTCTTCCCCCTGGCACCCTCCTCCAAGA |
| | | GCACCTCTGGGGGCACAGGGGCCCTGGGCTGCCTGGTCAAGGACTACTTC |
| | | CCCGAACCGGTGACGGTGTCGTGGAACTCAGGCGCCCTGACCAGCGGCGT |
| | | CCACACCTTCCCGGCTGTCCTACAGTCCTCAGGACTCTACTCCCTCAGCA |
| | | GCGTAGTGACCGTGCCGTCCAGCAGCTTGGGCACCCAGACCTACATCTGC |
| | | AACGTGAATCACAAGCCCAGCAACACCAAGGTGGACAAGAAAGTTGAGGC |
| | | CAAATGTTGT |
| 409 | Anti CD132 | CAGCTGCAGCTGCAGGAATCCGGGGGAGGCGTCGTCCAGCCAGGAAGGTC |
| | P2B2 scFv | ACTGAGACTGAGTTGTGCCGCAAGCGGGTTCACTTTCAGCTCCTACGCTA |
| | and Fc with hole | TGCACTGGGTGAGACAGGCACCCGGAAAGGGCCTGGAGTGGGTGGCAGTC |
| | modification | ATCTCTTATGACGGCGGGAACAAGTACTATGCCGATAGTGTGAAAGGCCG |
| | | GTTCACCATTAGTAGAGACAACTCAAAAAATACACTGTACCTCCAGATGA |
| | | ATAGCCTGCGCGCCGAAGACACAGGTGTGTACTATTGCGCAAAGTCCGTG |
| | | GCCCCCCCTATGGATGTGTGGGGGAAAGGAACCACAGTGACTGTCTCTAG |
| | | TGGAGGAGGAGGATCAGGCGGGGGAGGCAGGGGAGGAGGAGGGTCCGACG |
| | | TGGTCATGACTCAGTCCCCTCTGTCTCTGCCAGTGACCCCCGGCGAGCCT |
| | | GCTTCCATCTCTTGTAGGTCAAGCCAGAGCCTGCTGCACTCCAACGGGTA |
| | | CAATTATCTGGATTGGTACTTGCAGAAGCCAGGCCAGTCTCCCCATCTGC |
| | | TGATCTATCTGGGATCTAACAGGGCCAGTGGCGTGCCTGACCGCTTCAGT |
| | | GGCTCAGGGAGGGGAACTGATTTTACCCTGAAAATTAGCCGAGTCGAGGC |
| | | CGAAGATGTGGGCGTCTACTTCTGCATGCAGGCTCTGCGGACACCATATA |
| | | CTTTTGGCCAGGGGACCAAGCTGGAGATCAAGAACAGCGGCGGGGGCACC |
| | | GCGGCCGCGACTCACACATGCCCACCGTGCCCAGCACCTGAACTCCTGGG |
| | | GGGACCGTCAGTCTTCCTCTTCCCCCCAAAACCCAAGGACACCCTCATGA |
| | | TGTCCCGGACCCCTGAGGTCACATGCGTGGTGGTGGACGTGAGCCACGAA |
| | | GACCCTGAGGTCAAGTTCAACTGGTACGTGGACGGGGTGGAGGTGCATAA |
| | | TGCCAAGACAAAGCCGCGGGAGGAGCAGTACAACAGCACGTACCGTGTGG |
| | | TCAGCGTCCTCACCGTCCTGCACCAGGACTGGCTGAATGGGAAGGAGTAC |
| | | AAGTGCAAGGTCTCCAACAAAGCCCTCCCAGCCCCCATCGAGAAAACCAT |
| | | CTCCAAAGCCAAAGGGCAGCCCCGAGAACCACAGGTGTGCACCCTGCCCC |
| | | CATCCCGGGATGAGCTGACCAAGAACCAGGTCAGCCTGTCCTGCGCCGTC |
| | | AAAGGCTTCTATCCCAGCGACATCGCCGTGGAGTGGGAGAGCAATGGGCA |
| | | GCCGGAGAACAACTACAAGACCACGCCTCCCGTGCTGGACTCCGACGGCT |
| | | CCTTCTTCCTCGTGAGCAAGCTCACCGTGGACAAGAGCAGGTGGCAGCAG |
| | | GGGAACGTCTTCTCATGCTCCGTGATGCATGAGGCTCTGCACAACCACTA |
| | | CACGCAGAAGAGCCTCTCCCTGTCTCCGGGTAAA |
| 410 | Anti CD132 P2B7 Fab | GATGTTGTGATGACTCAGTCTCCACTCTCCCTGCCCGTCACCCTTGGACA |
| | LC (VL, joint CL) | GCCGGCCTCCATCTCCTGCAGGTCTAGTCAGAGCCTCGTCCATAGTAATG |
| | | GATACAACTATTTGGACTGGTACCTGCAGAAGCCAGGGCAGTCTCCACAG |
| | | CTCCTGATCTATTTGGGTTCTAATCGGGCCTGGGGGGTCCCTGACAGGTT |
| | | CAGTGGCAGTGGATCGGGCACAGATTTTACACTGAAAATCAGCAGAGTGG |
| | | AGGCTGAGGATGTTGGGGTTTATTACTGCCTGCAAGGTTCACACTGGCCT |
| | | TGGACGTTCGGCCAAGGGACCAAGGTGGAAATCAAACGAACTGTGGCTGC |
| | | ACCATCTGTCTTCATCTTCCCGCCATCTGATGAGCAGTTGAAATCTGGAA |
| | | CTGCCTCTGTTGTGTGCCTGCTGAATAACTTCTATCCCAGAGAGGCCAAA |
| | | GTACAGTGGAAGGTGGATAACGCCCTCCAATCGGGTAACTCCCAGGAGAG |
| | | TGTCACAGAGCAGGACAGCAAGGACAGCACCTACAGCCTCAGCAGCACCC |
| | | TGACGCTGAGCAAAGCAGACTACGAGAAACACAAAGTCTACGCCTGCGAA |
| | | GTCACCCATCAGGGCCTGAGCTCGCCCGTCACAAAGAGCTTCAACAGGGG |
| | | AGAGTGT |
| 411 | Anti CD132 P2B7 Fab | CAGGTGCAGCTACAGCAGTGGGGCGCAGGACTGTTGAAGCCTTCGGAGAC |
| | HC (VH, joint CH1) | CGTGTCCCTCACCTGCGCTGTGTATGGTGAGTCCTTCAGTGGTTACTACT |
| | | GGAGCTGGATCCGCCAGCCCCCAGGGAAGGGGCTGGAGTGGATTGGGGAA |
| | | ATCAATCATAGTGGAAGCACCAACTACAACCCGTCCCTCAAGAGTCGAGT |
| | | CACCATATCAGTAGACACGTCCAAGAACCAGTTCTCCCTGAAGCTGAGCT |
| | | CTGTGACCGCCGCGGACACGGCTGTGTATTACTGTGCGAGAGGCCCCGCG |
| | | GGTAGCAGCTCGTCCGGCTACTTTGACTACTGGGGCCAGGGAACCCTGGT |
| | | CACCGTCTCAAGCGCCTCCACCAAGGGCCCATCGGTCTTCCCCCTGGCAC |
| | | CCTCCTCCAAGAGCACCTCTGGGGGCACAGCGGCCCTGGGCTGCCTGGTC |
| | | AAGGACTACTTCCCCGAACCGGTGACGGTGTCGTGGAACTCAGGCGCCCT |
| | | GACCAGCGGCGTCCACACCTTCCCGGCTGTCCTACAGTCCTCAGGACTCT |
| | | ACTCCCTCAGCAGCGTAGTGACCGTGCCCTCCAGCAGCTTGGGCACCCAG |
| | | ACCTACATCTGCAACGTGAATCACAAGCCCAGCAACACCAAGGTGGACAA |
| | | GAAAGTTGAGCCCAAATCTTGT |
| 412 | Anti CD132 P2B7 scFv | CAGGTCCAGCTGCAGCAGTGGGGCGCCGGACTGGTGAAACCCTCTGAAAC |
| | and Fc with hole | TCTGAGCCTGACTTGTGCCGTCTATGGGGAATCCTTCTCTGGCTACTATT |
| | modification | GGAGTTGGATCAGGCAGCCCCCTGGCAAGGGGCTGGAGTGGATCGGAGAA |
| | | ATTAACCACAGCGGCTCCACCAACTACAATCCATCTCTGAAAAGTCGCGT |
| | | GACCATTTCCGTGGACACATCTAAGAATCAGTTCAGCCTGAAGCTGAGCA |
| | | GCGTGACAGCCGCTGATACTGCCGTCTACTATTGCGCACGGGGCCCCGCC |
| | | GGGTCTAGTTCAAGCGGATACTTTGACTATTGGGGACAGGGCACCCTGGT |
| | | GACAGTCTCCTCTGGGGGAGGAGGCTCCGGAGGAGGAGGGTCTGGAGGAG |
| | | GAGGAAGCGATGTGGTCATGACACAGTCACCACTGAGCCTGCCAGTGACT |
| | | CTGGGACAGCCTGCTTCTATCAGTTGTCGAAGTTCACAGAGTCTGGTCCA |
| | | CTCAAACGGATACAATTATCTGGACTGGTACTTGCAGAAGCCTGGCCAGA |
| | | GCCCACAGCTGCTGATCTATCTGGGGAGCAACCGAGCTTCCGGAGTGCCC |
| | | GACAGATTCTCAGGGAGCGGCAGCGGCACTGATTTTACCCTGAAAATTAG |
| | | CAGAGTGGAGGCAGAAGATGTGGGCGTCTACTATTGCCTCCAGGGGTCCC |
| | | ATTGGCCTTGGACTTTCGGGCAGGGAACCAAGGTGGAGATCAAGAACAGC |
| | | GGGGGGGGCACCGCGGCCGCGACTCACACATGCCCACCGTGCCCAGCACC |
| | | TGAACTCCTGGGGGGACCGTCAGTCTTCCTCTTCCCCCGAAAACCCAAGG |
| | | ACACCCTCATGATCTCCCGGACCCCTGAGGTCACATGCGTGGTGGTGGAC |
| | | GTGAGCCACGAAGACCCTGAGGTCAAGTTCAACTGGTACGTGGACGGCGT |
| | | GGAGGTGCATAATGCCAAGACAAAGCCGGGGGAGGAGCAGTACAACAGCA |
| | | CGTACCGTGTGGTCAGCGTCCTCACCGTCCTGCACCAGGACTGGCTGAAT |
| | | GGCAAGGAGTACAAGTGCAAGGTCTCCAACAAAGCCCTCCCAGCCCCCAT |
| | | CGAGAAAACCATCTCCAAAGCCAAAGGGCAGCCCCGAGAACCACAGGTGT |
| | | GCACCCTGCCCCCATCCCGGGATGAGCTGACCAAGAACCAGGTCAGCCTG |
| | | TCCTGCGCCGTCAAAGGCTTCTATCCCAGCGACATCGCCGTGGAGTGGGA |
| | | GAGCAATGGGCAGCCGGAGAACAACTACAAGACCACGCCTCCCGTGCTGG |
| | | ACTCCGACGGCTCCTTCTTCCTCGTGAGCAAGCTCACCGTGGACAAGAGC |
| | | AGGTGGCAGCAGGGGAACGTCTTCTCATGCTCCGTGATGCATGAGGCTCT |
| | | GCACAACCACTACACGCAGAAGAGCCTCTCCCTGTCTCCGGGTAAA |
| 413 | Anti CD132 P2D11 | GAAACGACACTCACGCAGTCTCCAGCCACCCTGTCTGTGTCTCCAGGGGA |
| | Fab LC | AAGAGCCACCCTCTCCTGCAGGGCCAGTCAGAGTGTTAGCAGCAACTTAG |
| | (VL, joint CL) | CCTGGTACCAGCAGAAACCTGGCCAGGCTCCCAGGCTCCTCATCTATGGT |
| | | GCATCCAGCGGGGCCACTGGCATCCCAGACAGGTTCAGTGGCAGTGGGTC |
| | | TGGGACAGACTTCACTCTCACCATCAGCAGACTGGAGCCTGAAGATTTTG |
| | | CAGTGTATTACTGTCAGGTGTATGGTAGCTCACTCGCTTTCGGGGGAGGG |
| | | ACCAAGGTGGAGATCAAACGAACTGTGGCTGCACCATCTGTCTTCATCTT |
| | | CCCGCCATCTGATGAGCAGTTGAAATCTGGAACTGCCTCTGTTGTGTGCC |
| | | TGCTGAATAACTTCTATCCCAGAGAGGCCAAAGTACAGTGGAAGGTGGAT |
| | | AACGCCCTCCAATCGGGTAACTCCCAGGAGAGTGTCACAGAGCAGGACAG |
| | | CAAGGACAGCACCTACAGCCTCAGCAGCACCCTGACGCTGAGCAAAGCAG |
| | | ACTACGAGAAACACAAAGTCTACGCCTGCGAAGTCACCCATCAGGGCCTG |
| | | AGCTCGCCCGTCACAAAGAGCTTCAACAGGGGAGAGTGT |
| 414 | Anti CD132 P2D11 | GAGGTCCAGCTGGTGCAGTCTGGGGCTGAGGTGAAGAAGCCTGGGTCCTC |
| | Fab HC | GGTGAAGGTCTCCTGCAAGGCTTCTGGAGGCACCTTCAGCAGCTATGCTA |
| | (VH, Joint CH1) | TCAGCTGGGTGCGACAGGCCCCTGGACAAGGGCTTGAGTGGATGGGATGG |
| | | ATCAGCGCTTACAATGGTGACACAAGCTACGCACAGAAGTTCCAGGGCAG |
| | | AGTCACCATTACCAGGGACACATCCGCGAGCACAGCCTACATGGAGCTGA |
| | | GCAGCCTGAGATCTGAAGACACGGCTGTGTATTACTGTGCGAGAGATTGG |
| | | GGATATTGTAGTGGTGGTAGCTGCTACCTGAACTGGTTCGACCCCTGGGG |
| | | CCAGGGAACCCTGGTCACCGTCTCAAGCGCCTCCACCAAGGGCCCATCGG |
| | | TCTTCCCCCTGGCACCCTCCTCCAAGAGCACCTCTGGGGGCACAGCGGCC |
| | | CTGGGCTGCCTGGTCAAGGACTACTTCCCCGAACCGGTGACGGTGTCGTG |
| | | GAACTCAGGCGCCCTGACCAGCGGCGTCCACACCTTCCCGGCTGTCCTAC |
| | | AGTCCTCAGGACTCTACTCCCTCAGCAGCGTAGTGACCGTGCCCTCCAGC |
| | | AGCTTGGGCACCCAGACCTACATCTGCAACGTGAATCACAAGCCCAGCAA |
| | | CACCAAGGTGGACAAGAAAGTTGAGCCCAAATCTTGT |
| 415 | Anti CD132 P2D11 | AGGTCCAGCTGCAGGAAAGCGGGCCAGGACTGGTCAAACCCTCACAGACA |
| | scFv and Fc with | CTGTCTCTGACTTGTACCGTCTCCGGGGGCTCAATCAGCTCCGGGGGGTA |
| | hole modification | CTATTGGACATGGATCAGACAGCACCCTGGACAGGGCCTGGAGTGGATCG |
| | | GGTTCATTAGCTGGTCCGGAACCACATACTATAACCCAAGCCTGAAGAAT |
| | | AGGGTGACAATTTCAGCCGACACTAGCAAAAACCATTTTTCCCTGAATCT |
| | | GACCTGTGTGAGAGCCGCTGATACTGCTGTCTACTATTGCGCACGGGGGT |
| | | CCGGAAGACTGGTGTGGGGACAGGGGACTCTGGTGACCGTCTCTAGTGGA |
| | | GGAGGAGGAAGTGGCGGAGGAGGCAGCGGAGGAGGAGGGTCCGAGACTAC |
| | | CCTGACCCAGTCTCCAGCTACACTGTCTGTGAGTCCCGGGGAAAGGGCAA |
| | | CCCTGAGCTGTCGCGCTTCACAGAGCGTCTCAAGCAACCTGGCATGGTAT |
| | | CAGCAGAAGCCTGGCCAGGCCCCTCGACTGCTGATCTATGGGGCATCCTC |
| | | TGGAGCCACTGGCATTCCCGACCGGTTCTCCGGATCTGGCAGTGGGACCG |
| | | ATTTTACACTGACCATCAGCCGGCTGGAGCCTGAAGACTTCGCTGTGTAC |
| | | TATTGCCAGCTGTACGGCAGTTCACTGGCATTTGGAGGGGGGACAAAGGT |
| | | CGAGATCAAGAACAGCGGGGGGGGCACCGCGGCCGCGACTCACACATGCC |
| | | CACCGTGCCCAGCACCTGAACTCCTGGGGGGACCGTCAGTCTTCCTCTTC |
| | | CCCCCAAAACCCAAGGACACCCTCATGATCTCCCGGACCCCTGAGGTCAC |
| | | ATGCGTGGTGGTGGACGTGAGCCACGAAGACCCTGAGGTCAAGTTCAACT |
| | | GGTACGTGGACGGCGTGGAGGTGCATAATGCCAAGAGAAAGCCGGGGGAG |
| | | GAGCAGTACAACAGCACGTACCGTGTGGTGAGCGTCGTCACCGTCCTGCA |
| | | CCAGGACTGGCTGAATGGCAAGGAGTACAAGTGCAAGGTCTCCAACAAAG |
| | | CCCTCCCAGCGCCCATCGAGAAAACCATCTCCAAAGCCAAAGGGCAGCCC |
| | | CGAGAACCACAGGTGTGCACCCTGCCCCCATCCCGGGATGAGCTGACCAA |
| | | GAACCAGGTCAGCCTGTCCTGCGCCGTCAAAGGCTTCTATCCCAGCGACA |
| | | TCGCCGTGGAGTGGGAGAGCAATGGGCAGCCGGAGAACAACTACAAGACC |
| | | ACGCCTCCCGTGCTGGACTCCGACGGCTCCTTCTTCCTCGTGAGCAAGCT |
| | | CACCGTGGACAAGAGCAGGTGGCAGCAGGGGAACGTCTTCTCATGCTCCG |
| | | TGATGCATGAGGCTCTGCACAACCACTACACGCAGAAGAGCCTCTCCCTG |
| | | TGTCCGGGTAAA |
| 416 | Anti CD132 P2F10 | GATATTGTGATGACCCACACTCCACTCTCCGTGCCCGTCACCCCTGGAGA |
| | Fab LC (VL, | GCCGGCCTCCATCTCGTGCAGGTCTAGTGAGACCCTCTTCGATAGCGATG |
| | joint CL) | ATGGAAAGACCTATTTGGACTGGTACCTGCAGAAGCCAGGGGAGTCTCCA |
| | | CAACTCCTGATGTATACCACTTCCTCTCGGGCCTCTGGAGTCCCAGACAG |
| | | GTTCAGTGGCAGTGGGTCAGGCACTGATTTCACACTGAAAATCAGCAGGG |
| | | TGGAGGCTGAGGATGTTGGAGTTTATTACTGCATGCAGCGTTTACAGTTT |
| | | CCCCTCACCTTCGGCCAAGGGACACGACTGGAGTTCAAACGAACTGTGGC |
| | | TGCACCATCTGTCTTCATCTTCCCGCCATGTGATGAGCAGTTGAAATCTG |
| | | GAACTGCCTCTGTTGTGTGCCTGCTGAATAACTTCTATCCCAGAGAGGCC |
| | | AAAGTACAGTGGAAGGTGGATAACGCCCTCCAATCGGGTAACTCCCAGGA |
| | | GAGTGTCACAGAGCAGGACAGCAAGGACAGCACCTACAGCCTCAGCAGCA |
| | | CCCTGACGCTGAGCAAAGCAGACTACGAGAAACACAAAGTCTACGCCTGC |
| | | GAAGTCACCCATCAGGGCCTGAGCTCGCCCGTCACAAAGAGCTTCAACAG |
| | | GGGAGAGTGT |
| 417 | Anti CD132 P2F10 | GAGGTCCAGCTGGTGCAGTCTGGGGCTGAGGTGAAGAAGCCTGGGGCCTC |
| | Fab HC | AGTGAAGGTCTCCTGCAAGGCTTCTGGATACACCTTCACCGGCTACTATA |
| | (VH, joint CH1) | TGCACTGGGTGCGACAGGCCCCTGGACAAGGGCTTGAGTGGATGGGAATA |
| | | ATCAACCCTAGTGGTGGTAGCACAAGCTACGCACAGAAGTTCCAGGGCAG |
| | | AGTCACCATGACCAGGGACACGTCCACGAGCACAGTCTACATGGAGCTGA |
| | | GCAGCCTGAGATCTGAGGACACGGCCGTGTATTACTGTGCGAGAGCCGAT |
| | | ACAGCTATGGGTGATGCTTTTGATATCTGGGGCCAAGGGACAATGGTCAC |
| | | CGTCTCAAGCGCCTCCACCAAGGGCCCATCGGTCTTCCCCCTGGCACCCT |
| | | CCTCCAAGAGCACCTCTGGGGGCACAGCGGCCCTGGGCTGCCTGGTCAAG |
| | | GACTACTTCCCCGAACCGGTGACGGTGTCGTGGAACTCAGGCGCCCTGAC |
| | | CAGCGGCGTCCACACCTTCCCGGCTGTCCTACAGTCCTCAGGACTCTACT |
| | | CCCTCAGCAGCGTAGTGACCGTGCCCTCCAGCAGCTTGGGCACCCAGACC |
| | | TACATCTGCAACGTGAATCACAAGCCCAGCAACACCAAGGTGGACAAGAA |
| | | AGTTGAGCCCAAATCTTGT |
| | | GAAGTCCAGCTGGTCCAGTCAGGAGCCGAGGTCAAGAAGCCAGGGGCAAG |
| | | CGTCAAAGTCTCATGCAAAGCAAGTGGGTACACATTTACAGGCTACTATA |
| | | TGCACTGGGTGAGGCAGGCTCCAGGACAGGGCCTGGAGTGGATGGGGATC |
| | | ATTAACCCCAGCGGCGGGAGTACCTCATACGCACAGAAGTTCCAGGGACG |
| | | GGTGACTATGACCAGAGACACAAGCACTTCCACCGTCTATATGGAGCTGA |
| | | GCAGCCTGCGATCCGAAGACAGTGCCGTGTACTATTCCGCCAGAGCCGAT |
| | | ACCGCAATGGGCGACGCCTTTGACATCTGGGGGCAGGGCACAATGGTGAC |
| | | AGTCTCTAGTGGAGGAGGAGGATCTGGAGGAGGAGGCAGTGGAGGAGGCG |
| | | GGTCAGACATCGTGATGACACATACTCCACTGTCTCTGCCAGTCACCCCT |
| | | GGCGAGCCAGCCTCTATTAGTTGTCGCTCAAGCCAGACCCTGTTCGACAG |
| | | TGACGATGGAAAGACATACCTGGATTGGTACTTGCAGAAACCTGGCCAGA |
| | | GCCCTCAGCTGCTGATGTACACCACATCCTCTAGGGCCTCCGGCGTGCCT |
| | | GACCGCTTCTCAGGCAGCGGGTCCGGAACTGATTTTACCCTGAAGATCAG |
| | | CCGGGTGGAGGCTGAAGACGTGGGGGTCTACTATTGCATGCAGAGACTCC |
| | | AGTTCCCACTGACATTTGGCCAGGGGACTCGGCTGGAGTTCAAGAACAGC |
| | | GGCGCGGGCACCGCGGCCGCGACTCACACATGCCCACCGTGCCCAGCACC |
| | | TGAACTCCTGGGGGGACCGTCAGTCTTCCTCTTCCCCCCAAAACCCAAGG |
| | | ACACCCTCATGATCTCCCGGACCCCTGAGGTCACATGCGTGGTGGTGGAC |
| | | GTGAGCCACGAAGACCCTGAGGTCAAGTTCAACTGGTACGTGGACGGCGT |
| | | GGAGGTGCATAATGCCAAGACAAAGCCGCGGGAGGAGCAGTACAACAGCA |
| | | CGTACCGTGTGGTCAGCGTCCTCACCGTCCTGCACCAGGACTGGCTGAAT |
| | | GGCAAGGAGTACAAGTGCAAGGTCTCCAACAAAGCCCTCCCAGCCCCCAT |
| | | CGAGAAAACCATCTCCAAAGCCAAAGGGCAGCCCCGAGAACCACAGGTGT |
| | | GCACCCTGCCCCCATCCCGGGATGAGCTGACCAAGAACCAGGTCAGCCTG |
| | | TCCTGCGCCGTCAAAGGCTTCTATCCCAGCGACATCGCCGTGGAGTGGGA |
| | | GAGCAATGGGCAGCCGGAGAACAACTACAAGACCACGCCTCCCGTGCTGG |
| | | ACTCCGACGGCTCCTTCTTCCTCGTGAGCAAGCTCACCGTGGACAAGAGC |
| | | AGGTGGCAGCAGGGGAACGTCTTCTCATGCTCCGTGATGCATGAGGCTCT |
| 418 | Anti CD132 | GCACAACCACTACACGCAGAAGAGCCTCTCCCTGTCTCCGGGTAAA |
| | P2F10 scFv and | |
| | Fc with hole | |
| | modification | |
| 419 | Anti CD132 | GATGTTGTGATGACTCAGTCTCCACTCTCCCTGCCCGTCACCCCTGGAGA |
| | P2H4 Fab | GCCGGCCTCCATCTCCTGCAGGGCAACTCAGAGCCTCCTGCATGGAAATG |
| | LC (VL, joint CL) | GACACAACTATTTGGATTGGTACCTGCAGAAGCCAGGGCAGTCTCCACAG |
| | | CTCCTGATCTATTTGGGTTCTAATCGGGCCTCCGGGGTCCCTGACAGGTT |
| | | CAGTGGCAGTGGATCAGGCACAGATTTTACACTGAAAATCAGCAGAGTGG |
| | | AGGCTGAGGATGTTGGGGTTTATTACTGCATGCAAACTCTGGAAACTCCT |
| | | GTCACTTTCGGCCCTGGGACCAAAGTGGATATCAAACGAACTGTGGCTGC |
| | | ACCATCTGTCTTCATCTTCCCGCCATCTGATGAGCAGTTGAAATCTGGAA |
| | | CTGCCTCTGTTGTGTGCCTGCTGAATAACTTCTATCCCAGAGAGGCCAAA |
| | | GTACAGTGGAAGGTGGATAACGCCCTCCAATCGGGTAACTCCCAGGAGAG |
| | | TGTCACAGAGCAGGACAGCAAGGACAGCACCTACAGCCTCAGCAGCACCC |
| | | TGACGCTGAGCAAAGCAGACTACGAGAAACACAAACTCTACGCCTGCGAA |
| | | GTCACCCATCAGGGCCTGAGCTCGCCCGTCACAAAGAGCTTCAACAGGGG |
| | | AGAGTGT |
| 420 | Anti CD132 | GAGGTCCAGCTGGTGCAGTGTGGGGGAGGCGTGGTCCAGCCTGGGAGGTC |
| | P2H4 Fab | CCTGAGACTCTCCTGTGCAGCCTCTGGATTCACCTTCAGTAGCTATGCTA |
| | HC (VH, joint CH1) | TGCACTGGGTCCGCCAGGCTCCAGGCAAGGGGCTGGAGTGGGTGGCAGTT |
| | | ATATCATATGATGGAAGCAATAAATACTACGCAGACTCCGTGAAGGGCCG |
| | | ATTCACCATCTCCAGAGACAATTCCAAGAACACGCTGTATCTGCAAATGA |
| | | ACAGCCTGAGAGCTGAGGACACGGCTGTGTATTACTGTGCGAGGTCTATC |
| | | GGTATCGGTGCTTTTGATATCTGGGGCCAAGGGACAATGGTCACCGTCTC |
| | | AAGCGCCTCCACCAAGGGCCCATCGGTCTTCCCCCTGGCACCCTCCTCCA |
| | | AGAGCACCTCTGGGGGCACAGCGGCCCTGGGCTGCCTGGTCAAGGACTAC |
| | | TTCCCCGAACCGGTGACGGTGTCGTGGAACTCAGGCGCCCTGACCAGCGG |
| | | CGTCCACACCTTCCCGGCTGTCCTACAGTCCTCAGGACTCTACTCCCTCA |
| | | GCAGCGTAGTGACCGTGCCCTCCAGCAGCTTGGGCACCCAGACCTACATC |
| | | TGCAACGTGAATCACAAGCCCAGCAACACCAAGGTGGACAAGAAAGTTGA |
| | | GCCCAAATCTTGT |
| 421 | Anti CD132 P2H4 | GAGGTCCAGCTGGTCCAGAGGGGGGGGGGGGTCGTGCAGCCTGGGAGAAG |
| | scFv and Fc | CCTGAGACTGTCCTGTGCCGCAAGCGGGTTTACTTTTAGCTCCTACGCTA |
| | with hole | TGCACTGGGTGAGGCAGGCACCCGGCAAGGGGCTGGAGTGGGTGGCAGTC |
| | modification | ATCTCCTATGACGGCTCTAACAAGTACTATGCCGATAGCGTGAAAGGGGG |
| | | GTTCACAATTAGTAGAGACAACTCAAAGAACACTCTGTACCTCCAGATGA |
| | | ATAGCCTGCGAGCCGAAGACACTGCTGTGTACTATTGCGCCCGGTCCATC |
| | | GGAATTGGGGCTTTTGACATCTGGGGGCAGGGCACAATGGTGACAGTCTC |
| | | TAGTGGAGGAGGAGGCTCTGGAGGAGGAGGGAGTGGAGGAGGAGGATCAG |
| | | ACGTGGTCATGACCCAGTCACCTCTGAGCCTGCCAGTGACACCTGGCGAG |
| | | CCAGCATCAATTAGCTGTAGAGCCACCCAGTGTCTGCTGCACGGCAAGGG |
| | | GCATAATTACCTGGATTGGTACTTGCAGAAGCCTGGCCAGAGTCCTCAGC |
| | | TGCTGATCTATCTGGGGAGCAACAGGGCTTCCGGAGTGCCAGACCGCTTC |
| | | TCCGGATCTGGCAGTGGGACTGATTTTACCCTGAAAATTTCCCGCGTCGA |
| | | GGCAGAAGACGTGGGAGTCTACTATTGCATGCAGACACTGGAAACTCCAG |
| | | TGACCTTCGGACCCGGCACAAAGGTGGACATCAAGAACAGCGGCGCGGGC |
| | | ACCGCGGCCGCGACTCACACATGCCCACCGTGCCCAGCACCTGAACTCCT |
| | | GGGGGGACCGTCAGTCTTCCTCTTCCCCCCAAAACCCAAGGACACCCTCA |
| | | TGATCTCCCGGACCCCTGAGGTCACATGCGTGGTGGTGGACGTGAGCCAC |
| | | GAAGACCCTGAGGTCAAGTTCAACTGGTACGTGGACGGCGTGGAGGTGCA |
| | | TAATGCCAAGACAAAGCCGGGGGAGGAGCAGTACAACAGCACGTACCGTG |
| | | TGGTCAGCGTCCTCACCGTCCTGCACCAGGACTGGCTGAATGGCAAGGAG |
| | | TACAAGTGGAAGGTCTCCAACAAAGCCCTCCCAGCCCCCATCGAGAAAAC |
| | | CATCTCCAAAGCCAAAGGGCAGCCCCGAGAACCACAGGTGTGCACCCTGC |
| | | CCCCATCCCCGGATGAGCTGACCAAGAACCAGGTCAGCCTGTCCTGCGCC |
| | | GTCAAAGGCTTCTATCCCAGCGACATCGCCGTGGAGTGGGAGAGCAATGG |
| | | GCAGCCGGAGAACAACTACAAGACCACGCCTCCCGTGCTGGACTCCGACG |
| | | GCTCCTTCTTCCTCGTGAGCAAGCTCACCGTGGACAAGAGCAGGTGGCAG |
| | | CAGGGGAACGTCTTCTCATGCTCCGTGATGCATGAGGCTCTGCACAACCA |
| | | CTACACGCAGAAGAGCCTCTCCGTGTCTCCGGGTAAA |
| 422 | Anti CD132 | GATGTTGTGATGACTCAGTCTCCACTCTCCCTGCCCGTCACCCCTGGAGA |
| | P2D3 Fab | GCCGGCCTCCATCTCCTGCAGGTCTAGTCAGAGCCTCCTGCATAGTAATG |
| | LC (VL, joint CL) | GATACAACTATTTGGATTGGTACCTGCAGAAGCCAGGGCAGTCTCCACAG |
| | | CTCCTGATCTATTTGGGTTCTAATCGGGCCTCCGGGGTCCCTGACAGGTT |
| | | CAGTGGCAGTGGATCAGGCACAGATTTTACACTGAAAATGAGCAGGGTGG |
| | | AGGCTGAGGATGTTGGGGTTTATTACTGCATGCAAGGTACACACTGGCCC |
| | | TGGACGTTCGGCCAAGGGACCAAGGTGGAAATCAAACGAACTGTGGCTGC |
| | | ACCATCTGTCTTCATCTTCCCGCCATCTGATGAGCAGTTGAAATCTGGAA |
| | | CTGCCTCTGTTGTGTGCCTGCTGAATAATTTCTATCCCAGAGAGGCCAAA |
| | | GTACAGTGGAAGGTGGATAACGCCCTCCAATGGGGTAACTCCCAGGAGAG |
| | | TGTCACAGAGCAGGACAGCAAGGACAGCACCTACAGCCTCAGCAGCACCC |
| | | TGACGCTGAGCAAAGCAGACTACGAGAAACACAAACTCTACGCCTGCGAA |
| | | GTCACCCATCAGGGCCTGAGCTCGCCCGTCAGAAAGAGCTTCAACAGGGG |
| | | AGAGTGT |
| 423 | Anti CD132 P2D3 | CAGGTGCAGCTACAGCAGTGGGGCGCAGGACTGTTGAAGCCTTCGGAGAC |
| | Fab HC | CGTGTCCCTCACCTGCACTATCTATGGTGGGTCCTTCAGTGGTTTCTACT |
| | (VH, joint CH1) | GGAGCTGGATCCGCCAGCCCCCAGGGAAGGGACTGGAGTGGATTGGGGAA |
| | | ATCAATCATAGTGGAAGCACCAACTACAACCCGTCCCTCAAGAGTCGAGT |
| | | CACCATATCAGTAGACACGTCCAAGAACCAGTTCTCCCTGAAGCTGAGCT |
| | | CTGTGACCGCCGCGGACACGGCTATATATTACTGTGCGAGAGGCCCCGGG |
| | | GGATCCACCTCGTCCGGGTACTTTGACCACTGGGGCCAGGGAACCCTGGT |
| | | CACCGTCTCAAGCGCCTCCACCAAGGGCCCATCGGTCTTCCCCCTGGCAC |
| | | CCTCCTCCAAGAGCACCTCTGGGGGCACAGCGGCCCTGGGCTGCCTGGTC |
| | | AAGGACTACTTCCCCGAACCGGTGACGGTGTCGTGGAACTCAGGGGCCCT |
| | | GACCAGCGGCGTCCACACCTTCCCGGCTGTCCTACAGTCCTCAGGACTCT |
| | | ACTCCCTCAGCAGCGTAGTGACCGTGCCCTCCAGCAGCTTGGGCACCCAG |
| | | ACCTACATCTGCAACGTGAATCACAAGCCCAGCAACACCAAGGTGGACAA |
| | | GAAAGTTGAGCCCAAATCTTGT |
| 424 | Anti CD132 P2D3 | CAGGTCCAGCTGCAGCAGTGGGGAGCCGGACTGCTGAAACCCTCTGAGAC |
| | scPv and Fc | TCTGAGCCTGACTTGCACAATCTACGGGGGATCATTCAGCGGCTTCTACT |
| | with hole | CGTCCTGGATCAGGCAGCCCCCTGGCAAGGGGCTGGAGTGGATCGGAGAA |
| | modification | ATTAACCACAGTGGCTCAACAAACTATAATCCCAGCCTGAAATCCCGCGT |
| | | GACCATCTCAGTGGACACAAGCAAGAATCAGTTCAGCCTGAAGCTGAGCA |
| | | GCGTGACAGCCGCTGATACTGCCATCTACTATTGCGCACGGGGCCCTGCC |
| | | GGGTCCACCTCTAGTGGGTACTTTGACCATTGGGGACAGGGCACCCTGGT |
| | | GACAGTCTCAAGCGGAGGAGGAGGCTCTGGAGGAGGAGGGAGTGGAGGGG |
| | | GGGGCAGCGATGTGGTCATGACTCAGTCTCCACTGAGTCTGCCAGTGACC |
| | | CCCGGCGAGCCTGCTAGCATCTCCTGTCGATCCTCTCAGTCCCTGCTGCA |
| | | CTCTAACGGATACAATTATCTGGACTGGTACTTGCAGAAGCCAGGCCAGA |
| | | GCCCCCAGCTGCTGATCTATCTGGGGAGTAACCGGGCTTCAGGAGTGCCT |
| | | GACAGATTCTCTGGGAGTGGATCAGGCACTGATTTTACCCTGAAAATTAG |
| | | CAGAGTCGAGGCAGAAGATGTGGGCGTCTACTATTGCATGCAGGGGACTC |
| | | ATTGGCCCTGGACCTTTGGGCAGGGAACAAAGGTGGAGATCAAGAACAGG |
| | | GGCGCGGGCACCGCGGCCGCGACTCACACATGCCCACCGTGCCCAGCACC |
| | | TGAACTCCTGGGGGGACCGTCAGTCTTCCTCTTCCCCCCAAAACCCAAGG |
| | | ACACCCTCATGATCTCCCGGACCCCTGAGGTCACATGCGTGGTGGTGGAC |
| | | GTGAGCCACGAAGACCCTGAGGTCAAGTTCAACTGGTACGTGGACGGCGT |
| | | GGAGGTGCATAATGCCAAGACAAAGCCGGGGGAGGAGCAGTACAACAGCA |
| | | CGTACCGTGTGGTCAGCGTCCTCACCGTCCTGCACCAGGACTGGCTGAAT |
| | | GGCAAGGAGTACAAGTGCAAGGTCTCCAACAAAGCCCTCCCAGCCCCCAT |
| | | CGAGAAAACCATCTCCAAAGCCAAAGGGCAGCCCCGAGAACCACAGGTGT |
| | | GCACCCTGCCCCCATCCCGGGATGAGCTGACCAAGAACCAGGTCAGCCTG |
| | | TCCTGCGCCGTCAAAGGCTTCTATCCCAGCGACATCGCCGTGGAGTGGGA |
| | | GAGCAATGGGCAGCCGGAGAACAACTACAAGACCACGCCTCCCGTGCTGG |
| | | ACTCCGACGGCTCCTTCTTCCTCGTGAGCAAGCTGACCGTGGACAAGAGC |
| | | AGGTGGCAGCAGGGGAACGTCTTCTCATGCTCCGTGATGCATGAGGCTCT |
| | | GCACAACCACTACACGCAGAAGAGCCTCTCCCTGTGTCCGGGTAAA |
| 425 | Anti CD132 P1G4 | GATGTTGTGATGACTCAGTCTCCACTCTCCGTGCCCGTCACCCCTGGAGA |
| | Fab LC | GCCGGCCTCCATCTCCTGCAGGTCTAGTCAGAGCCTCCTGCATAGTAATG |
| | (VL, joint CL) | GATACAACTATTTGGATTGGTACCTGCAGAAGCCAGGGCAGTCTCCACAG |
| | | CTCCTGATCTATTTGGGTTCTAATCGGGCCTCCGGGGTCCCTGACAGGTT |
| | | CAGTGGCAGTGGATCAGGCACAGATTTTACACTGAAAATCAGCAGGGTGG |
| | | AGGCTGAGGATGTTGGGGTTTATTACTGCCTGCAAGGTACACATTGGCCG |
| | | TGGACGTTCGGCCAGGGGACCAAGGTGGAAATCAAACGAACTGTGGCTGC |
| | | ACCATGTGTCTTCATCTTCCCGCCATCTGATGAGCAGTTGAAATGTGGAA |
| | | CTGCCTCTGTTGTGTGCCTGCTGAATAACTTCTATCCCAGAGAGGCCAAA |
| | | GTACAGTGGAAGGTGGATAACGCCCTCCAATCGGGTAACTCCCAGGAGAG |
| | | TGTCACAGAGCAGGACAGCAAGGACAGCACCTACAGCCTCAGCAGCACCC |
| | | TGACGCTGAGCAAAGCAGACTACGAGAAACACAAAGTCTACGCCTGCGAA |
| | | GTCACCCATCAGGGCCTGAGCTCGCCCGTCACAAAGAGCTTCAACAGGGG |
| | | AGAGTGT |
| 426 | Anti CD132 P1G4 | CAGGTGCAGCTACAGCTGTGGGGCGCAGGACTGTTGAAGCCTTCGGAGAC |
| | Fab HC | CCTGTCCCTCACCTGCGCTGTCTATGGTGGGTCCCTCAGTGGTTACTACT |
| | (VH, joint CH1) | GGAGCTGGATCCGCCAGCCCCCAGGGAAGGGGCTGGAGTGGATTGGGGAA |
| | | ATCAATCATAGTGGAAGCACCAACTACAACCCATCCCTCAAGAGTCGAGT |
| | | CACCATATCAGTAGACACGTCCAAGAACCAGTTCTCCCTGAAGCTGAGCT |
| | | CTGTGACCGCCGCGGACACGGCTGTGTATTACTGTGCGAGAGGCAGCAGC |
| | | TCCTACTACATGGACGTCTGGGGCAAAGGGACCACGGTCACCGTCTCAAG |
| | | CGCCTCCACCAAGGGCCCATCGGTCTTCCCCCTGGCACCCTCCTCCAAGA |
| | | GGACCTCTGGGGGCACAGCGGCCCTGGGCTGCCTGGTCAAGGACTACTTC |
| | | CCCGAACCGGTGACGGTGTCGTGGAACTCAGGCGCCCTGACCAGCGGCGT |
| | | CCACACCTTCCCGGCTGTCCTACAGTCCTCAGGACTCTACTCCCTCAGCA |
| | | GCGTAGTGACCGTGCCCTCCAGCAGCTTGGGCACCCAGACCTACATCTGC |
| | | AACGTGAATCACAAGCCCAGCAACACCAAGGTGGACAAGAAAGTTGAGCC |
| | | CAAATCTTGT |
| | | CAGGTCCAGCTGCAGCAGTGGGGAGCCGGACTGCTGAAACCAAGCGAGAC |
| | | TCTGAGCCTGACTTGTGCCGTGTATGGGGGAAGCCTGTCCGGCTACTATT |
| | | GGTCTTGGATGAGGCAGCCCCCTGGCAAGGGGCTGGAGTGGATCGGCGAA |
| | | ATTAACCACTCAGGGAGCACAAACTACAATCCCTCCCTGAAATCTCGCGT |
| | | GACCATTAGCGTGGACACATCCAAGAATGAGTTCAGCCTGAAGGTGAGCA |
| | | GCGTGACAGGCGCTGAGACCGCGGTGTACTATTGCGCCAGAGGCAGCAGC |
| | | AGCTACTATATGGATGTGTGGGGAAAGGGGACCACAGTGACCGTCAGCTC |
| | | CGGAGGAGGAGGCAGTGGAGGAGGAGGGTCCGGAGGCGGGGGATCTGACG |
| | | TGGTCATGACTCAGAGTCCTCTGTCACTGCCTGTGACCCCCGGGGAGCCT |
| | | GCATCCATGTCTTGTCGATCTAGTCAGTGTCTGCTGCACAGTAACGGCTA |
| | | CAATTATCTGGATTGGTACTTGCAGAAGCCAGGGCAGTCCCCCCAGCTGC |
| | | TGATCTATCTGGGATCAAACCGGGCTAGCGGCGTGCCTGACAGATTCAGT |
| | | GGGTCAGGAAGCGGCACTGATTTTACCCTGAAAATTAGCAGAGTCGAGGG |
| | | AGAAGATGTGGGGGTCTACTATTGCCTCCAGGGAACTCATTGGCCCTGGA |
| | | CCTTTGGGCAGGGAACAAAGGTGGAGATCAAGAACAGCGGGGGGGGCACC |
| | | GCGGCCGCGACTCACACATGCCCACCGTGCCCAGCACCTGAACTCCTGGG |
| | | GGGACCGTCAGTCTTCCTCTTCCCCCCAAAACCCAAGGACACCCTCATGA |
| | | TCTCCCGGACCCCTGAGGTCACATGCGTGGTGGTGGACGTGAGCCACGAA |
| | | GACCCTGAGGTCAAGTTCAACTGGTACGTGGACGGCGTGGAGGTGCATAA |
| | | TGCCAAGACAAAGCCGCGGGAGGAGCAGTACAACAGCACGTACCGTGTGG |
| | | TCAGCGTCCTCACCGTCCTGCACCAGGACTGGCTGAATGGCAAGGAGTAC |
| | | AAGTGCAAGGTCTCCAACAAAGCCCTCCCAGCCCCCATCGAGAAAACCAT |
| | | CTCCAAAGCCAAAGGGCAGCCCCGAGAACCACAGGTGTGCACCCTGCCCC |
| | | CATCCCGGGATGAGCTGACCAAGAACCAGGTCAGCCTGTCCTGCGCCGTC |
| | | AAAGGCTTCTATCCCAGCGACATCGCCGTGGAGTGGGAGAGCAATGGGCA |
| | | GCCGGAGAACAACTACAAGACCACGCCTCCCGTGCTGGACTCCGACGGCT |
| | | CCTTCTTCCTCGTGAGCAAGCTCACCGTGGACAAGAGCAGGTGGCAGCAG |
| | | GGGAACGTCTTCTCATGCTCCGTGATGCATGAGGCTCTGCACAACCACTA |
| 427 | Anti CD132 P1G4 | CACGCAGAAGAGCCTCTCCGTGTCTCCGGGTAAA |
| | scFv and Fc | |
| | with hole | |
| | modification | |
| 428 | Anti CD132 P1B12 | GATGTTGTGATGACTCAGTCTCCACTCTCCCTGCCCGTCACCCTTGGTCA |
| | Fab LC (VL, joint CL) | GCCGGCCTCCATCTCCTGCAGGTCTAGTCAGAGCCTCCTGCACAGTAATG |
| | | GAAACAACTATTTGGATTGGTACCTGGAGAAGCCAGGGCAGTCTCCACAG |
| | | CTCCTGATCTATTTGGGTTCTAATCGGGCCTCGGGGGTCCCTGACAGGTT |
| | | CAGTGGCAGTGGATCAGGCACAGATTTTACACTGAAAATCAGCAGGGTGG |
| | | AGGCTGAGGATGTTGGGATTTATTACTGCATGCAAGGGACACACTGGCCT |
| | | TGGACGTTCGGCCAAGGGACCAAGGTGGAAATCGAACGAACTGTGGCTGC |
| | | ACCATCTGTCTTCATCTTCCCGCCATCTGATGAGCAGTTGAAATCTGGAA |
| | | CTGCCTCTGTTGTGTGCCTGCTGAATAACTTCTATCCCAGAGAGGCCAAA |
| | | GTACAGTGGAAGGTGGATAGCGCCCTCCAATCGGGTAACTCCCAGGAGAG |
| | | TGTCACAGAGCAGGAGAGGAAGGACAGCACCTACAGCCTCAGCAGCACCC |
| | | TGACGCTGAGCAAAGCAGACTACGAGAAACACAAACTCTACGCCTGCGAA |
| | | GTCACCCATCAGGGCCTGAGCTCGCCCGTCACAAAGAGCTTCAACAGGGG |
| | | AGAGTGT |
| 429 | Anti CD132 P1B12 | CAGGTGCAGCTACAGCAGTGGGGCGCAGGACTGTTGAAGCCTTCGGAGAC |
| | Fab HC | CCTGTCCCTCACCTGCGCTGTCTATGGTGGGTCCTTCAGTGGTTAGTACT |
| | (VH, joint CH1) | GGAGCTGGATCCGCCAGCCCCCAGGGAAGGGGCTGGAGTGGATTGGGGAA |
| | | ATCAATCATAGTGGAAGCACCAACTACAACCCGTCCCTCAAGAGTCGAGT |
| | | CACCATATCAGTAGACACGTCCAAGAACCAGTTCTCCCTGAAGCTGAGCT |
| | | GTGTGACCGCCGCGGACACGGCTGTGTATTACTGTGCGAGAGGCGGTAGC |
| | | GCGTACTTCCAGCACTGGGGCCAGGGAACCCTGGTCACCGTCTCAAGCGC |
| | | CTCCACCAAGGGCCCATCGGTCTTCCCCCTGGCACCCTCGTCCAAGAGCA |
| | | CCTCTGGGGGCACAGCGGCCCTGGGCTGCCTGGTCAAGGACTACTTCCCC |
| | | GAACCGGTGACGGTGTCGTGGAACTCAGGCGCCCTGACCAGCGGCGTCCA |
| | | CACCTTCCCGGCTGTCCTACAGTCCTCAGGACTCTACTCCCTCAGCAGCG |
| | | TAGTGACCGTGCCCTCCAGCAGCTTGGGCACCCAGACCTACATCTGCAAC |
| | | GTGAATCACAAGCCCAGCAACACCAAGGTGGACAAGAAAGTTGAGCCCAA |
| | | ATCTTGT |
| 430 | Anti CD132 P1B12 scFv | CAGGTCCAGCTGCAGCAGTGGGGGGGGGGGGTGCTGAAACCTTCCGAAAC |
| | and Fc with hole | TCTGTCTCTGACTTGTGCCGTGTATGGGGGGTCCTTTAGTGGCTACTATT |
| | modification | GGTCATGGATCAGGCAGCCCCCTGGAAAGGGCCTGGAGTGGATCGGAGAA |
| | | ATTAACCACTGGGGCTCTACAAACTAGAATCCAAGTCTGAAATCACGCGT |
| | | GACCATTTCTGTGGACACCAGTAAGAATCAGTTCAGCCTGAAGCTGAGCA |
| | | GCGTGACAGCCGCTGATACCGCCGTGTACTATTGCGCCCGAGGCGGGTCT |
| | | GCTTATTTTCAGCATTGGGGGCAGGGAACCCTGGTGACAGTCTCTAGTGG |
| | | AGGAGGAGGCAGCGGCGGAGGAGGCTCTGGAGGAGGAGGGAGTGACGTGG |
| | | TCATGACTCAGAGCCCACTGTCCCTGCCAGTGACCCTGGGACAGCCAGCT |
| | | AGTATCTCATGTAGATCAAGCCAGTCACTGCTGCACAGCAACGGCAACAA |
| | | TTACCTGGATTGGTACTTGCAGAAGCCTGGCCAGAGCCCACAGCTGCTGA |
| | | TCTACCTGGGGTCCAATCGGGCATCTGGAGTGCCCGACAGATTCAGCGGC |
| | | TCCGGGTCTGGAACTGATTTTACCCTGAAGATCAGCCGGGTGGAGGCCGA |
| | | AGACGTCGGCATCTACTATTGCATGCAGGGGACTCATTGGCCTTGGACCT |
| | | TCGGCCAGGGGACAAAAGTGGAGATCGAAAACAGGGGCGCGGGCACCGCG |
| | | GCCGCGACTCACACATGCCCACCGTGCCCAGCACCTGAACTCCTGGGGGG |
| | | ACCGTCAGTCTTCCTCTTCCCCCCAAAACCCAAGGACACCCTCATGATCT |
| | | CCCGGACCCCTGAGGTCACATGCGTGGTGGTGGACGTGAGCCACGAAGAC |
| | | CCTGAGGTCAAGTTCAACTGGTACGTGGACGGCGTGGAGGTGCATAATGC |
| | | CAAGACAAAGCCGCGGGAGGAGCAGTACAACAGCACGTACCGTGTGGTCA |
| | | GCGTCCTCACCGTCCTGCACCAGGACTGGCTGAATGGCAAGGAGTACAAG |
| | | TGCAAGGTCTCCAACAAAGCCCTCCCAGCCCCCATCGAGAAAACCATGTC |
| | | CAAAGCCAAAGGGCAGCCCCGAGAACCACAGGTGTGCACCCTGCCCCCAT |
| | | CCCGGGATGAGCTGACCAAGAACCAGGTCAGCCTGTCCTGCGCCGTCAAA |
| | | GGCTTCTATCCCAGCGACATCGCCGTGGAGTGGGAGAGCAATGGGCAGCC |
| | | GGAGAACAACTACAAGACCACGCCTCCCGTGCTGGACTCCGACGGCTCCT |
| | | TCTTCCTCGTGAGCAAGCTCACCGTGGACAAGAGCAGGTGGCAGCAGGGG |
| | | AACGTCTTCTCATGCTCCGTGATGCATGAGGCTCTGCACAACCACTACAC |
| | | GGAGAAGAGCCTCTCCCTGTCTCCGGGTAAA |
| 433 | Anti CD132 P1C7 | GAAATTGTGCTGACTCAGTCTCCACTCTCCCTGCCCGTCACCCCTGGAGA |
| | Fab LC (VL, joint CL) | GCCGGCCTCCATCTCCTGCAGGTCTAGTCAGAGCCTCCTGCATAGTAATG |
| | | GATACAACTATTTGGATTGGTACCTGCAGAAGCCAGGGCAGTCTCCACAG |
| | | CTCCTGATCTATTTGGCTTCTAATCGGGCCTCCGGGGTCCCAGACAGATT |
| | | CAGCGGCAGTGGGTCAGGCACTGATTTCACACTGAAAATCAGCAGGGTGG |
| | | AGGCTGAGGATGTTGGGGTTTATTACTGCATGCAAGGTACACACTGGCCG |
| | | TGGACGTTCGGCCAAGGGACCAAGGTGGAAGTCAAACGAACTGTGGCTGC |
| | | ACCATCTGTCTTCATCTTCCCGCCATGTGATGAGCAGTTGAAATCTGGAA |
| | | CTGCCTCTGTTGTGTGCCTGCTGAATAACTTCTATCCCAGAGAGGCCAAA |
| | | GTACAGTGGAAGGTGGATAACGCCCTCCAATCGGGTAACTCCCGGGAGAG |
| | | TGTCACAGAGCAGGACAGCAAGGACAGCACCTACAGCCTCAGCAGCACCC |
| | | TGAGGCTGAGGAAAGCAGACTACGAGAAACACAAAGTCTACGCCTGCGAA |
| | | GTCACCCATCAGGGCCTGAGCTCGCCGGTCACAAAGAGCTTCAACAGGGG |
| | | AGAGTGT |
| 432 | Anti CD132 P1C7 | CAGGTGCAGCTACAGCAGTGGGGCGCAGGACTGTTGAAGCCTTCGGAGAC |
| | Fab HC | CGTGTCCCTCACCTGCGCTGTCTATGGTGGGTCCTTCAGTGGTTACTACT |
| | (VH, joint CH1) | GGAGCTGGATCCGCCAGCCCCCAGGGAAGGGGCTGGAGTGGATTGGGGAA |
| | | ATCAATCATAGTGGAAGCACCAACTACAACCCGTCCCTCAAGAGTCGAGT |
| | | CACCATATCAGAAGACGCGTCCAAGAAGCAGTTCTCCCTGACGCTGACCT |
| | | CTGTGACCGCCGCGGACACGGCTGTCTATTACTGTGCGAGAGGCCCCGCG |
| | | GGTACCGGCTCGTCCGGCTACTTTGACTACTGGGGCCAGGGAACCCTGGT |
| | | CACCGTCTCAAGCGCCTCCACCAAGGGCCCATCGGTCTTCCCCCTGGCAC |
| | | CCTCCTCCAAGAGCACCTCTGGGGGCACAGCGGCCCTGGGCTGCCTGGTC |
| | | AAGGACTACTTCCCCGAACCGGTGACGGTGTCGTGGAACTCAGGCGCCCT |
| | | GACCAGCGGCGTCCACACCTTCCCGGCTGTCCTACAGTCCTCAGGACTCT |
| | | ACTCCCTCAGCAGCGTAGTGACCGTGCCCTCCAGCAGCTTGGGCACCCAG |
| | | ACCTACATCTGCAACGTGAATCACAAGCCCAGCAACACCAAGGTGGACAA |
| | | GAAAGTTGAGCCCAAATCTTGT |
| 433 | Anti CD132 P1C7 | CAGGTCCAGCTGCAGCAGTGGGGAGCCGGACTGCTGAAGCCTAGCGAAAC |
| | scFv and Fc | TCTGAGCCTGACTTGTGCTGTCTACGGAGGATCATTTAGTGGCTACTATT |
| | with hole | GGTCATGGATCAGGCAGCCCCCTGGCAAGGGGCTGGAGTGGATCGGAGAA |
| | modification | ATTAACCACTCCGGCTCTACAAACTACAATCCCAGTCTGAAATCACGCGT |
| | | GACTATTTCTGAGGACGCCAGTAAGAAACAGTTCTCCCTGACCCTGACAT |
| | | CTGTGAGCGCCGCTGATACAGCTGTCTACTATTGCGCACGGGGCCCTGCC |
| | | GGAACAGGCAGCTCCGGATACTTTGACTATTGGGGGCAGGGAACTCTGGT |
| | | GACCGTCTCTAGTGGGGGAGGAGGCAGTGGAGGAGGAGGGTCCGGAGGAG |
| | | GAGGATCTGAGATCGTGCTGACTCAGAGCCCACTGTCCCTGCCAGTCACC |
| | | CCCGGCGAACCTGCCAGTATTTCATGTCGATCAAGCCAGTCACTGCTGCA |
| | | CAGCAACGGATACAATTATCTGGACTGGTACTTGCAGAAGCCAGGCCAGA |
| | | GCCCCCAGCTGCTGATCTATCTGGCTTCCAATCGGGCATCTGGCGTGCCT |
| | | GACAGATTCAGGGGCTCCGGGTCTGGAACAGATTTTACTCTGAAAATTTC |
| | | CAGAGTGGAGGCCGAAGATGTGGGGGTCTACTATTGCATGCAGGGAACTC |
| | | ATTGGCCCTGGACCTTCGGCCAGGGGACAAAGGTGGAAGTCAAAAACAGC |
| | | GGCGCGGGCACCGCGGCCGCGACTCACACATGCCCACCGTGCCCAGCACC |
| | | TGAACTCCTGGGGGGACCGTCAGTCTTCGTCTTCCCCCCAAAACCCAAGG |
| | | ACACCCTCATGATCTCCCGGACCCCTGAGGTCACATGCGTGGTGGTGGAC |
| | | GTGAGCCACGAAGACCCTGAGGTCAAGTTCAACTGGTACGTGGACGGCGT |
| | | GGAGGTGCATAATGCCAAGACAAAGCCGCGGGAGGAGCAGTACAACAGCA |
| | | CGTACCGTGTGGTCAGCGTCCTCACCGTCCTGCACCAGGACTGGCTGAAT |
| | | GGCAAGGAGTACAAGTGCAAGGTCTCCAACAAAGCCCTCCCAGCCCCCAT |
| | | CGAGAAAACCATCTCCAAAGCCAAAGGGCAGCCCCGAGAACCACAGGTGT |
| | | GCACCCTGCCCCCATCCCGGGATGAGCTGACCAAGAACCAGGTCAGCCTG |
| | | TCCTGCGCCGTCAAAGGCTTCTATCCCAGCGACATCGCCGTGGAGTGGGA |
| | | GAGCAATGGGCAGCCGGAGAACAACTACAAGACCACGCCTCCCGTGCTGG |
| | | ACTCCGACGGCTCCTTCTTCCTCGTGAGCAAGCTCACCGTGGACAAGAGC |
| | | AGGTGGCAGCAGGGGAACGTCTTCTCATGCTCCGTGATGCATGAGGGTCT |
| | | GCACAACCACTACACGCAGAAGAGCCTCTCCCTGTCTCCGGGTAAA |
| 434 | Humans CD122 | MAAPALSWRLPLLILLLPLATSWASAAVNGTSQFTCFYNSRANISCVWSQ |
| | (UniProt: P14784 1, v1) | DGALQDTSCQVHAWPDRRRWNQTCELLPVSQASWACNLILGAPDSQKLTT |
| | | VDIVTLRVLCREGVRWRVMAIQDFKPFENLRLMAPISLQVVHVETHRGNI |
| | | SWEISQASHYFERHLEFEARTLSPGHTWEEAPLLTLKQKQEWICLETLTP |
| | | QTQYEFQVRVKPLQGEFTTWSPWSQPLAFRTKPAALGKDTIPWLGHLLVG |
| | | LSGAFGFIILVYLLINCANTGPWLKKVLKQNTPDPSKFFSQLSSEHGGDV |
| | | QKWLSSPFPSSSFSPGGLAPEISPLEVLERDKVTQLLLQQDKVPEPASLS |
| | | SNHSLTSCFTNQGYFFFHLPDALEIEACQVYFTYDPYSEEDPDEGVAGAR |
| | | TGSSPQPLQPLSGEDDAYCTFPSRDDLLLFSPSLLGGPSPPSTAPGGSGA |
| | | GEERMPPSLQERVPRQWDPQPLGPPTPGVPDLVDFQPPPELVLREAGEEV |
| | | PDAGPREGVSFPWSRPPGQGEFRALNARLPLNTDAYLSLQELQGQDPTHL |
| | | V |
| 435 | Mature form Human | AVNGTSQFTCFYNSRANISCWWSQDGALQDTSCQVHAWPDRRRWNQTCEL |
| | CD122 (UniProt: P14784 1, | LPVSQASWACNLILGAPDSQKLTTVDIVTLRVLCREGVRWRVMAIQDFKP |
| | v1 residues 27 to 525) | FENLRLMAPISLQVVHVETHRQNISWEISQASHYFERHLEFEARTLSPGH |
| | | TWEEAPLLTLKQKQEWICLETLTPDTQYEPQVRVKPLQGEFTTWSPWSQP |
| | | LAFRTKPAALGKDTIPWIGHLLVGLSGAFGFIILVYLLINCRNTGPWLKK |
| | | VLKCNTPDPSKFFSQLSSEHGGDVQKWLSSPFPSSSPSPGGLAPEISPLE |
| | | VLERDKVTQLLLQQDKVPEPASLSSNHSLYSCFTNQGYFFFHLPDALEIE |
| | | ACQVYFTYDPYSEEDPDEGVAGAPTGSSPQPLQPLSGEDDAYCTFPSRDD |
| | | LLLFSPSLLGGPSPPSTAPGGSGAGEERMPPSLQERVPRDWDPQPLGPPT |
| | | PGVPDLVDFQPPPELVLREAGEEVPDAGPREGVSFPWSRPPGQGEFRALN |
| | | ARLPLNTDAYLSLQELQGQDPTHLV |
| 436 | Extracelular domain of | AVNGTSQFTCFYNSRANISCWWSQDGALQDTSCQVHAWPDRRRWNQTCEL |
| | Human CD122 | LPVSQASWACNLIILGAPDSQKLTTVDIVTLRVLCREGVRWRVMAIQDFK |
| | (UniProt: P14784 1, | PFENLRLMAPISLQVVHVETHRCNISWEISQASHYFERHLEFEARTLSPG |
| | v1 residues 27 to 240) | HTWEEAPLLTLKQKQEWICLETLTPDTQYEFQVRVKPLQGEFTTWSPWSQ |
| | | PLAFRTKPAALGKDT |
| 437 | Human CD132 | MLKPSLPFTSLLFLQLPLLGVGLNTTILTPNGNEDTTADFFLTTMPTDSL |
| | (UniProt: P31785 1, V1) | SVSTLPLPEVQCFVFNVEYMNCTWNSSSEPQPTNLTLHYWYKNSDNDKVQ |
| | | KCSHYLFSEEITSGCQLQKKEIHLYQTFVVQLQDPREPRRQATQMLKLQN |
| | | EVIPWAPENLTLHKLSESQLELNWNNRFLNHCLEHLVQYRTDWDHSWTEQ |
| | | SVDYRHKFSLPSVDGQKRYTFRVRSRENPLCGSAQHWSEWSHPIHWGSNT |
| | | SKENPFLFALEAVVISVGSMGLIISLLCVYFWLERTMPRIPTLKNLEDLV |
| | | TEYHGNFSAWSGVSKGLAESLQPDYSERLCLVSEIPPKGGALGEGPGASP |
| | | QNQHSPYWAPPCYTLKPET |
| 438 | Mature form Human | LNTTILTPNGNEDTTADFFLTTMPTDSLSVSTLPLPEVQCFVFNVEYMNC |
| | CD132 (UniProt: P31785 1, | TWNSSSEPQPTNLTLHYWYKNSDNDKVQKCSHYLFSEEITSGCQLQKKEI |
| | v1 residues 23 to 369) | HLYQTFVVQLQDPREPRRQATQMLKLQNLVIPWAPENLTLHKLSESQLEL |
| | | NWNNRFLNHCLEHLVQYRTDWDHSWTEQSVDYRHKFSLPSVDGQKRYTFR |
| | | VRSRFNPLCGSAQHWSEWSHPIHWGSNTSKENPFLFALEAVVISVGSMGL |
| | | IISLLCVYFWLERTMPRIPTLKNLEDLVTEYHGNFSAWSGVSKGLAESLQ |
| | | PDYSERLCLVSEIPPKGGALGEGPGASPCNQHSPYWAPPCYTLKPET |
| 439 | Extracellular domain of | LNTTILIPNGNEDTTADFFLTTMPTDSLSVSTLPLPEVQCFVENVEYMNC |
| | Human CD132 | TWNSSSEPQPTNLTLHYWYKNSDNDKVQKCSHYLFSEEITSGCQLQKKEI |
| | (UniProt: P31785 1, | HLYQTFVVQLQDPREPRRQATQMLKLQNLVIPWAPENLTLHKLSESQLEL |
| | V1 residues 23 to 262) | NWNNRFLNHCLEHLVQYRTDWDHSWTEQSVDYRHKFSLPSVDGQKRYTFR |
| | | VRSRFNPLCGSAQHWSEWSHPIHWGSNTSKENPFLFALEA |
| 440 | Human IgG1 constant | ASTKGPSVFPLAPSSKSTSGGTAALGCLVKDYFPEPVTVSWNSGALTSGV |
| | region IGHG1; | HTFPAVLQSSGLYSLSSVVTVPSSSLGTQTYICNVNHKPSNTKVDKKVEP |
| | UniProt: P01857 1, v1) | KSCDKTHTCPPCPAPELLGGPSVFLFPPKPKDTLMISRTPEVTCVVVDVS |
| | | HEDPEVKFNWYVDGVEVHNAKTKPREEQYNSTYRVVSVLTVLHQDWLNGK |
| | | EYKCKVSNKALPAPIENTISKAKGQPREPQVYTLPPSRDELTKNQVSLTC |
| | | LVKGFYPSDIAVEWESNGQPENNYKTIPPVLDSDGSPFLYSKLIVDKSRW |
| | | QQQNVFSCSVMHEALHNHYTQKSLSLSPGK |
| 441 | CHif IgG1 (positions | ASTKGPSVFPLAPSSKSTSGGTAALGCLVKDYFPEPVTVSWNSGALTSGV |
| | 1 96 of P01887 1, v1) | HTFPAVLQSSGLYSLSSVVTVPSSSLGTQTYICNVNHKPSNTKVDKKV |
| 442 | Hinge IgG1 (positions | EPKSCDKTHTCP |
| | 99 110 of P01857 1, v1) | |
| 443 | CH2 IgG1 | PCPAPELLGGPSVFLFPPKPKDTLMISRTPEVTCVVVDVSHEDPEVKFNW |
| | (positions 111 223 of | YVDGVEVHNAKTKPREEQYNSTYRVVSVLTVLHQDWLNGKEYKCKVSNKA |
| | P01857 1, v1) | LPAPIEKTISKAK |
| 444 | CH3 IgG1 | GQPREPQVYTLPPSRDELTKNQVSLTCLVKGFYPSDIAVEWESNGQPENN |
| | (positions 224 330 | YKTTPPVLDSDGSFFLYSKLTVDKSRWQQGNVFSCSVHEALHNHYTQKSL |
| | of P01857 1, v1) | SLSPGK |
| 445 | CK CL (IGCK UniProt: | RTVAAPSVFIFPPSDEQLKSGTASVVCLLNNFYPREAKVQWKVDNALQSG |
| | P01834 1, v2) | NSQESVTEQDSKDSTYSLSSTLTLSKADYEKHKVYACEVTHQGLSSPVTK |
| | | SFNRGEC |
| 446 | CH2 CH3 IgG1 | PCPAPELLGGPSVFLFPPKPKDTLMISRTPEVTCVVVDVSHEDPEVKFNW |
| | (positions 111 330 | YVDGVEVHNAKTKPREEQYNSTYRVVSVLTVLHQDWLNGKEYKCKVSNKA |
| | of P01857 1, v1) | LPAPIEKTISKAKGQPREPQVYTLPPSRDELTKNQVSLTCLVKGFYPSDI |
| | | AVEWESNGQPENNYKTTPPVLDSDGSFFLVSKLTVDKSRWQQGNVFSCSV |
| | | MHEALHNMYTQKSLSLSPGK |
| 447 | CH3 (T386W, 8354C) | GQPREPQVYTLPPCRDELTKNQVSLWCLVKGFYPSDIAVEWESNGQPENN |
| | | YKTTPPVLDSDGSFFLYSKLTVDKSRWQQGNVFSCSVMHEALHNMYTQKS |
| | | LSLSPGK |
| 448 | CH3 (T366, | GQPREPQVCTLPPSRDELTKNQVSLSCAVKGFYPSDIAVEWESNGQPENN |
| | L368A, Y407V, Y349C) | YKTTPPVLDSDGSFFLVSKLTVDKSRWQQGNVFSCSVMHEALHNMYTQKS |
| | | LSLSPGK |
| 449 | CH2 CH3 (T366W, S354C) | PCPAPELLGGPSVFLFPPKPKDTLMISRTPEVTCVVVDVSHEDPEVKFNW |
| | | YVDGVEVHNAKTKPREEQYNSTYRVVSVLTVLHQDWLNGKEYKCKVSNKA |
| | | LPAPIEKTISKAKGQPREPQVYTLPPCRDELIKNQVSLSCAVKGFYPSDD |
| | | AVEWESNGQPENNYKTTPPVLDSSGSFFLYSKLTVDKSRWQQGNVFSCSV |
| | | HEALHNHYTQKSLSLSPGK |
| 450 | CH2 CH3 (T386S, L366A, | PCPAPELLGGPSVFLFPPKPKDTLMISRTPEVTCVVVDVSHEDPEVKFNW |
| | Y407V, Y349C) | YVDGVEVHNAKTKPREEQYNSTYRVVSVLTVLHQDWLNGKEYKCKVSNKA |
| | | LPAPIEKTISKAKGQPREPQVCILPPSRDELTKNQVSLSCAVKGFYPSDI |
| | | AVEWESNGQPENNYKTYPPVLDSDGSFFLVSKLTVDKSRWQQGNVFSCSV |
| | | HEALHNHYTQKSLSLSPGK |
| 451 | CH2(LALA) CH3 | PCPAPEAAGGPSVFLFPPKPKDTLMISRTPEVTCVVVDVSHEDPEVKFNW |
| | | YVDGVEVHNAKTKPREEQYNSTYRVVSVLTVLHQDWLNGKEYKCKVSNKA |
| | | LPAPIEKTISKAK |
| 452 | CH2(LALA) CH3 | PCPAPEAAGGPSVFLFPPKPKDTLMISRTPEVTCVVVDVSHEDPEVKFNW |
| | (T366W, S354C) | YVDGVEVHNAKTKPREEQYNSTYRVVSVLTVLHQDWLNGKEYKCKVSNKA |
| | | LPAPIEKTISKAKGQPREEPQVYTLPPCRDELTKNQVSLWCLVKGFYPSD |
| | | IAVEWESNGQPENNYKTTPPVLDSDGSFFLYSKLTVDKSRWQQGNVFSCS |
| | | VHEALHNHYTQKSLSLSPGK |
| 453 | CH2(LALA) CH3 (T366S, | PCPAPEAAGGPSVFLFPPKPKDTLMISRTPEVTCVVVDVSHEDPEVKFNW |
| | L366A, Y407V, Y349C) | YVDGVEVHNAKTKPREEQYNSTYRVVSVLTVLHQDWLNGKEYKCKVSNKA |
| | | LPAPIEKTISKAKGQPREPQVCTLPPSRDELTKNQVSLSCAVKGFYPSDI |
| | | AVEWESNGQPENNYKTTPPVLDSDGSFFLVSKLTVDKSRWQQGNVFSCSV |
| | | HEALHNHYTQKSLSLSPGK |
| 454 | Linker 5 | GGGGSGGGGSGGGGSGGGGS |
| 455 | Linker 6 | GGGGS |

The invention includes the combination of the aspects and preferred features described except where such a combination is clearly impermissible or expressly avoided.

The section headings used herein are for organizational purposes only and are not to be construed as limiting the subject matter described.

Aspects and embodiments of the present invention will now be illustrated, by way of example, with reference to the accompanying figures. Further aspects and embodiments will be apparent to those skilled in the art. All documents mentioned in this text are incorporated herein by reference.

Throughout this specification, including the claims which follow, unless the context requires otherwise, the word “comprise,” and variations such as “comprises” and “comprising.” will be understood to imply the Inclusion of a stated integer or step or group of integers or steps but not the exclusion of any other integer or step or group of integers or steps.

It must be noted that, as used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by the use of the antecedent “about,” it will be understood that the particular value forms another embodiment.

Where a nucleic acid sequence is disclosed herein, the reverse complement thereof is also expressly contemplated.

Methods described herein may preferably performed in vitro. The term “in vitro” is intended to encompass experiments with cells in culture whereas the term “in vivo” is intended to encompass experiments with intact multi-cellular organisms.

### BRIEF DESCRIPTION OF THE FIGURES

Embodiments and experiments illustrating the principles of the invention will now be discussed with reference to the accompanying figures.

FIGS. 1A and 18. Graphs showing binding of different formats of bispecific anti-IL2RB/γc antibodies to (1A) γc-Fc and (1B) IL2RG-Fc as determined by ELISA.

FIGS. 2A to 2C. Graphs and bar chart showing binding of bispecific and monospecific IL2Rβ-add/or γc-binding antibodies to cells expressing human IL2Rβ, γc or IL-2Rα at the cell surface, as determined by flow cytometry, (2A) Graphs showing analysis of binding of P2C4/P1A3, P2C4/P1A10, a monospecific anti-γc (‘neg/αIL2Rγ’), a monospecific anti-IL-2RB (‘αIL2Rβ/neg’) to cells transfected with constructs encoding human IL-2Rβ and γc. Negative unstained, secondary antibody only and isotype control conditions are indicated (28) Graphs showing analysis of binding of P2C4/P1A3, P2C4/P1A10, a monospecific anti-γc (‘neg/αIL2Rγ’), a monospecific anti-IL-2Rβ (‘αIL2R⊕/neg’) to cells transfected with construct encoding IL-2Rα. Negative unstained, secondary antibody only and isotype control conditions, and positive αIL2Rα control conditions are indicated. (2C) Bar chart summarising normalised median fluorescence intensity (nMFI) for binding of the indicated antibodies to cells transfected with constructs encoding IL-2Rβ and γc.

FIGS. 3A and 38. Bar charts showing binding of bispecific IL-2Rβ- and γc-binding antibodies to primary human T cell subsets, as determined by flow cytometry. (3A and 3B) Bar chart summarising normalised MFIs for binding of the indicated antibodies to the indicated CD4+(3A) and CD8+(3B) human T cell subsets.

FIGS. 4A and 48. Graphs and bar chart showing binding of bispecific and monospecific IL-2Rβ- and/or γc-binding antibodies to cells expressing rhesus IL-2Rβ and γc at the cell surface, as determined by flow cytometry. (4A) Graphs showing analysis of binding of P2C4/P1A3, P2C4/P1A10, a monospecific anti-γc (‘neg/αIL2Rγ’), a monospecific anti-IL-2Rβ ((‘αIL2RB/neg’) to cells transfected with constructs encoding rhesus IL-2Rβ and γc. Negative unstained, secondary antibody only and isotype control conditions are indicated. (48) Bar chart summarising normalised MFIs for binding of the indicated antibodies to cells transfected with constructs encoding rhesus IL-2Rβ and γc.

FIG. 5. Bar chart showing binding of bispecific IL-2Rβ- and γc-binding antibodies to primary cynomolgus macaque T cell subsets, as determined by flow cytometry.

FIGS. 6A and 68. Graphs showing analysis of proliferation of NK92 cells in response to treatment with bispecific IL-2Rβ- and γc-binding antibodies or the indicated cytokines. EC50 values for induction of NK92 cell proliferation are shown. 6A and 6B show the results from different experiments investigating different bispecific IL-2Rβ- and γc-binding antibodies.

FIGS. 7A to 7L. Bar charts and graphs showing analysis of proliferation of pre-activated, primary human T cell subsets in response to treatment with bispecific IL-2Rβ- and γc-binding antibodies or IL-2 Unstimulated cells (media) and anti-CD3/CD28 bead-stimulated controls (beads) are indicated. (7A)

Absolute numbers of CD4+ T cells. (7B) Absolute numbers of CD8+ T cells. (7C) Absolute numbers of Tregs. (7D) Graphs showing CD4+CD25+FoxP3+ regulatory T cell compartment following stimulation with the indicated agents. (7E) Absolute numbers of naive CD8+ T cells. (7F) Absolute numbers of naive CD4+ T cells. (7G) Absolute numbers of central memory CD8+ T cells. (7H) Absolute numbers of central memory CD4+ T cells. (7I) Absolute numbers of effector memory CD8+ T cells. (7J) Absolute numbers of effector memory CD4+ T cells. (7K) Graphs showing dividing effector memory CD8+ T cells as determined by CellTrace Violet staining. (7L) Percentage of CD8+ effector memory cells which are dividing.

FIGS. 8A to 8H. Graphs showing analysis of proliferation of pre-activated, primary human T cell subsets in response to treatment with different amounts of bispecific IL-2Rβ- and γc-binding antibodies of the indicated cytokines. (8A) Absolute numbers of CD4+ T cells. (8B) Absolute numbers of CD8+ T cells. (8C) Absolute numbers of Tregs. (8D) Ratio of the absolute number of CD8+ T cells to the absolute number of Tregs. (8E) Absolute numbers of effector memory CD4+ T cells. (8F) Absolute numbers of effector memory CD8+ T cells. (8G) Percentage of CD8+ effector memory cells which are dividing. Unstimulated cells (media) and anti-CD3/CD28 bead-stimulated controls (beads) are indicated. (8H) Absolute numbers of pre-expanded Tregs after treatment with bispecific IL-2Rβ- and γc-binding antibodies or the indicated cytokines.

FIGS. 9A to 9I. Bar charts showing analysis of proliferation of pre-activated, T cell subsets in response to treatment of human PBMCs with bispecific IL-2Rβ- and γc-binding antibodies or IL-2 Unstimulated cells (media) and anti-CD3/CD28 bead-stimulated controls (beads) are indicated. (9A) Absolute numbers of CD4+ T cells (9B) Absolute numbers of CD8+ T cells. (9C) Absolute numbers of Tregs. (9D) Absolute numbers of naïve CD8+ T cells. (9E) Absolute numbers of naïve CD4+ T cells. (9F) Absolute numbers of central memory CD8+ T cells. (9G) Absolute numbers of central memory CD4+ T cells. (9H) Absolute numbers of effector memory CD8+ T cells. (9I) Absolute numbers of effector memory CD4+ T cells.

FIGS. 10A to 10G. Bar charts and graphs showing analysis of proliferation of antigen-specific T cells in response to treatment with bispecific IL-2Rβ- and γc-binding antibodies or the indicated cytokines. Unstimulated cells (media) and anti-CD3/CD28 bead-stimulated controls (beads) are indicated. (10A and 10D) Absolute numbers of CD4+EBV-specific T cells. (10B and 10E) Absolute numbers of CD8+EBV-specific T cells. (10C) Absolute numbers of CD56+EBV-specific T cells. (10F) Percentage of CD8+EBV-specific T cells which are dividing. (10G) Graphs showing dividing C08+EBV-specific T cells as determined by CellTrace Violet staining.

FIG. 11A to 11L. Bar charts showing analysis of proliferation of cynomolgus T cell subsets in response to treatment of cynomolgus PBMCs with bispecific IL-2Rj3- and ye-binding antibodies or IL-2. Unstimulated cells (media) and anti-C03/C02B bead-stimulated controls (beads) are indicated. (11A) Absolute numbers of C04+ T cells. (11B) Absolute numbers of COB+ T cells (11C) Absolute numbers of Tregs. (110) Absolute numbers of naive C04+ T cells. (11E) Absolute numbers of effector memory C04+ T cells. (11F) Absolute numbers of central memory C04+ T cells. (11G) Absolute numbers of naYve COB+ T cells. (11H) Absolute numbers of effector memory C08+ T cells (111) Absolute numbers of central memory C08+ T cells. (11J) Absolute numbers of NK cells. (11K) Absolute numbers of B cells. (11L) Ratio of the absolute number of COB+ T cells to the absolute number of C04+ T cells.

FIG. 12A to 12N. Bar charts showing analysis of proliferation of pre-activated cynomolgus T cell subsets in response to treatment of cynomolgus PBMCs with bispecific IL-2Rj3- and ye-binding antibodies or IL-2. Unstimulated cells (media) and anti-C03/C028 bead-stimulated controls (beads) are indicated. (12A) Absolute numbers of C04+ T cells. (12B) Absolute numbers of C08+ T cells. (12C) Absolute numbers of Tregs. (120) Absolute numbers of na′ive C04+ T cells. (12E) Absolute numbers of effector memory C04+ T cells. (12F) Absolute numbers of central memory C04+ T cells. (12G) Absolute numbers of naYve C08+ T cells. (12H) Absolute numbers of effector memory C08+ T cells. (121) Absolute numbers of central memory C08+ T cells (12J) Ratio of the absolute number of C08+ T cells to the absolute number of C04+ T cells. (12K) Percentage of effector memory C04+ T cells which are dividing. (12L) Percentage of central memory C04+ T cells which are dividing. (12M) Percentage of effector memory COB+ T cells which are dividing. (12N) Percentage of central memory COB+ T cells which are dividing.

FIG. 13. Graph showing analysis of induction of STAT5 phosphorylation in NK92 cells in response to treatment with bispecific IL-2RI3- and ye-binding antibodies or IL-2. EC50 values for induction of STAT5 phosphorylation are shown.

FIGS. 14A to 14H. Graphs showing analysis of induction of STAT5 phosphorylation in human immune cell subsets following treatment of PBMCs with different amounts of bispecific IL-2Rj3- and ye-binding antibodies or IL-2. EC50 values for induction of STAT5 phosphorylation are shown. (14A) Percentage pSTAT5-positive naYve C04+ T cells. (14B) Percentage pSTAT5-positive memory C04+ T cells. (14C) Percentage pSTAT6-positive Tregs (140) Percentage pSTAT5-positive 8 cells. (14E) Percentage pSTAT5-positive na ive C08+ T cells. (14F) Percentage pSTAT5-positive memory C08+ T cells. (14G) Percentage pSTAT5-positive NK cells. (14H) Percentage pSTAT5-positive monocytes.

FIGS. 15A to 15C. Graphs showing analysis of induction of STAT5 phosphorylation in human Immune cell subsets following treatment of pre-activated PBMCs with different amounts of bispecific IL-2Rj3- and ye-binding antibodies or IL-2. EC50 values for induction of STAT5 phosphorylation are shown. (15A) Percentage pSTAT5-positive C04+ T cells. (15B) Percentage pSTAT5-positive COB+ T cells (15C) Percentage pSTAT5-positive NK cells.

FIG. 16. Western blot showing kinetics of induction of STATE phosphorylation in NK92 cells following treatment with bispecific IL-2Rβ- and γc-binding antibodies or IL-2. Total STAT6 and actin were included as controls.

FIGS. 17A to 17E. Graphs showing kinetics of induction of STAT5 phosphorylation in human immune call subsets following treatment of PBMCs with bispecific IL-2Rβ- and γc-binding antibodies or IL-2. (17A) Percentage pSTAT5-positive T cells. (17B) Percentage pSTAT5-positive CD8+ T cells (17C) Percentage pSTAT5-positive CD4+ T cells. (17D) Percentage pSTAT5-positive monocytes. (17E) Percentage pSTAT5-positive B cells.

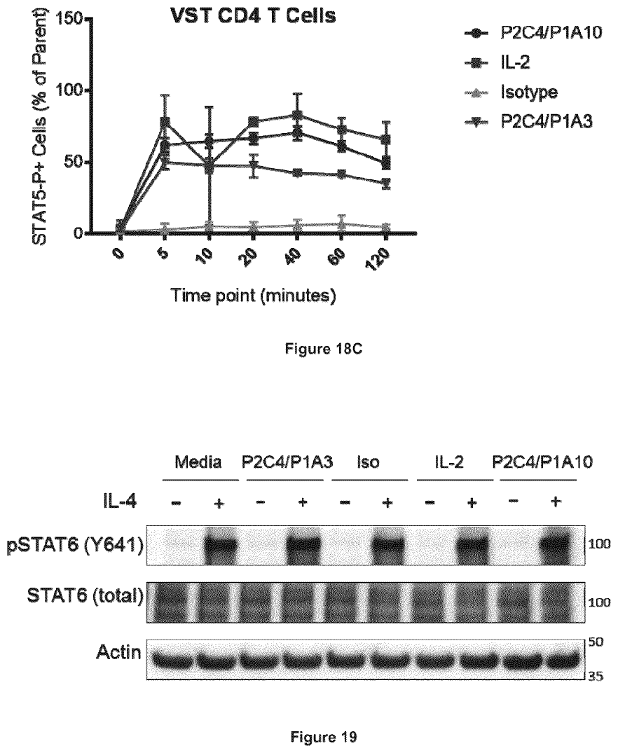

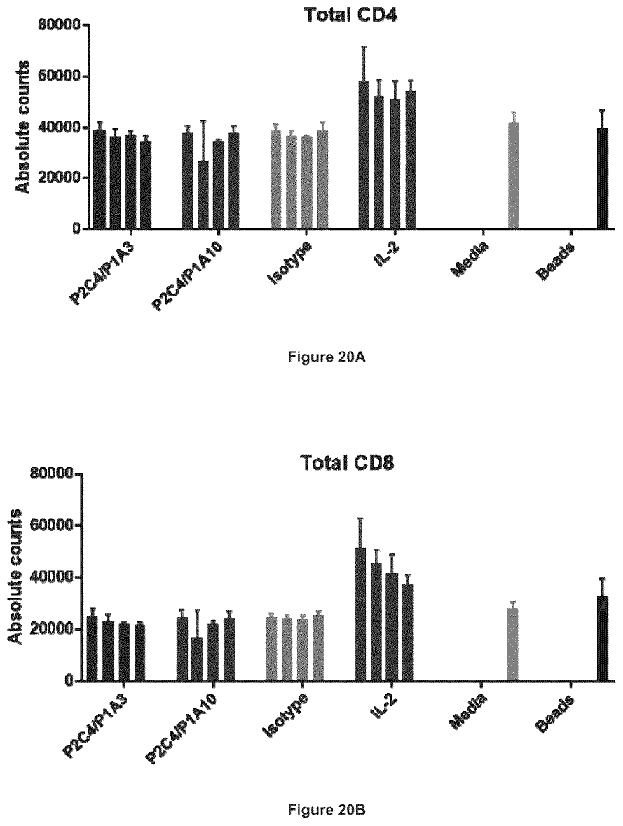

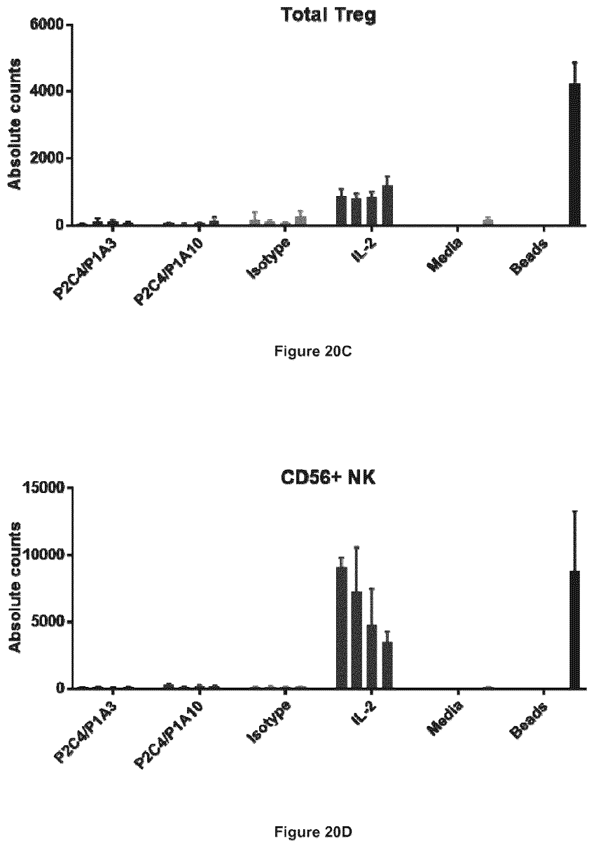

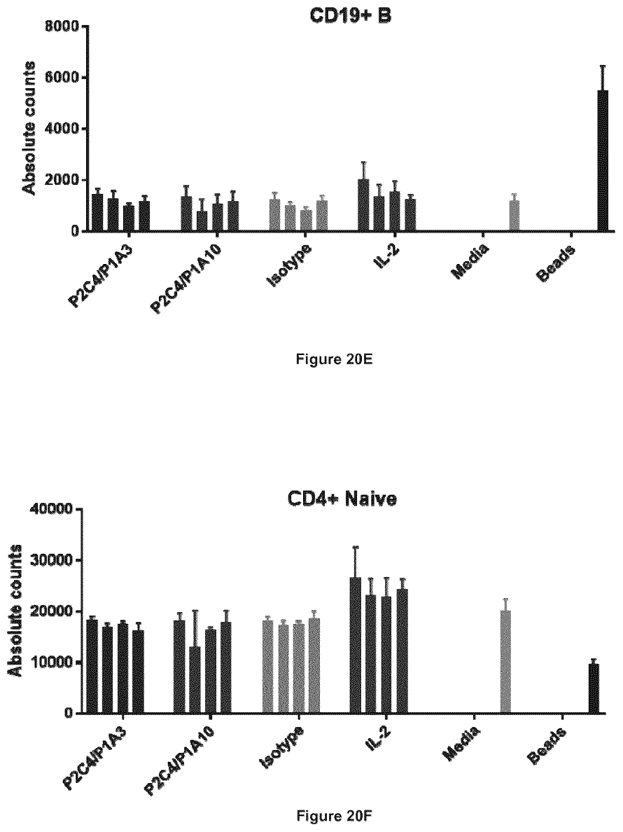

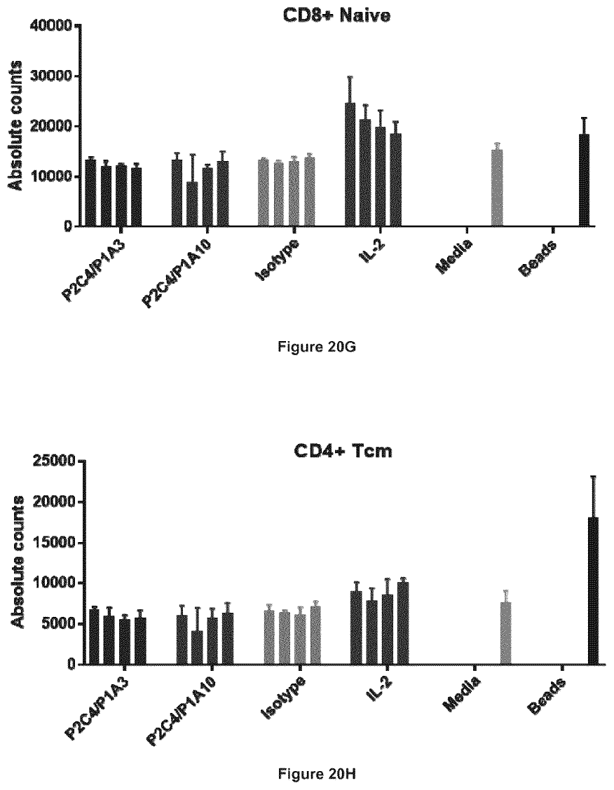

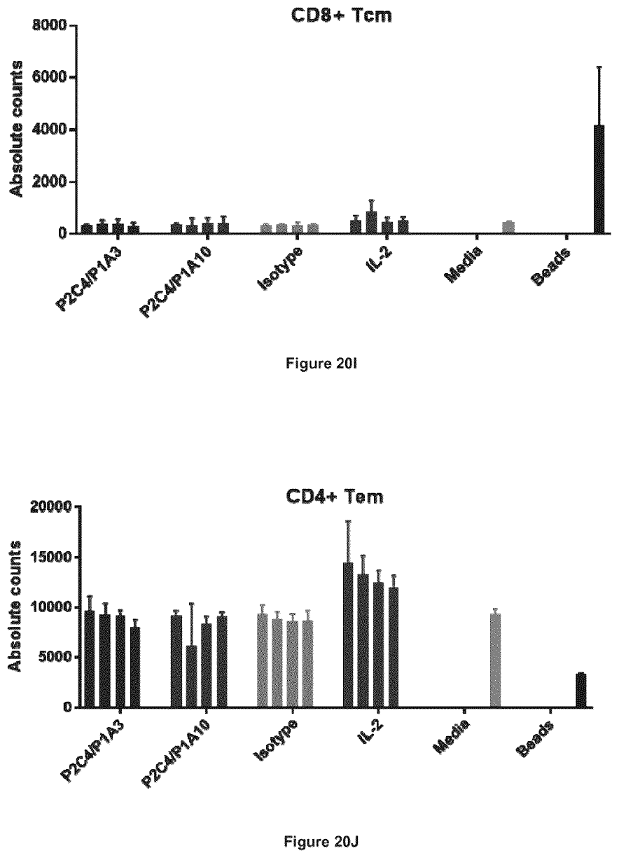

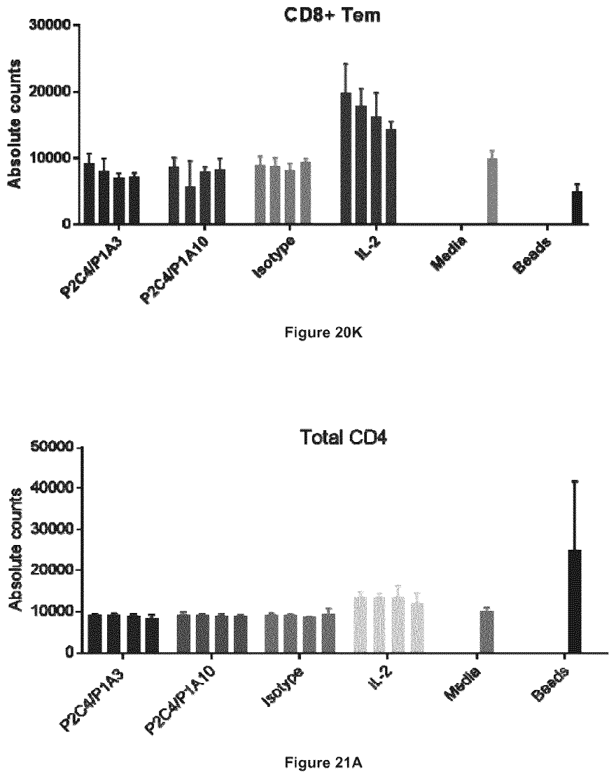

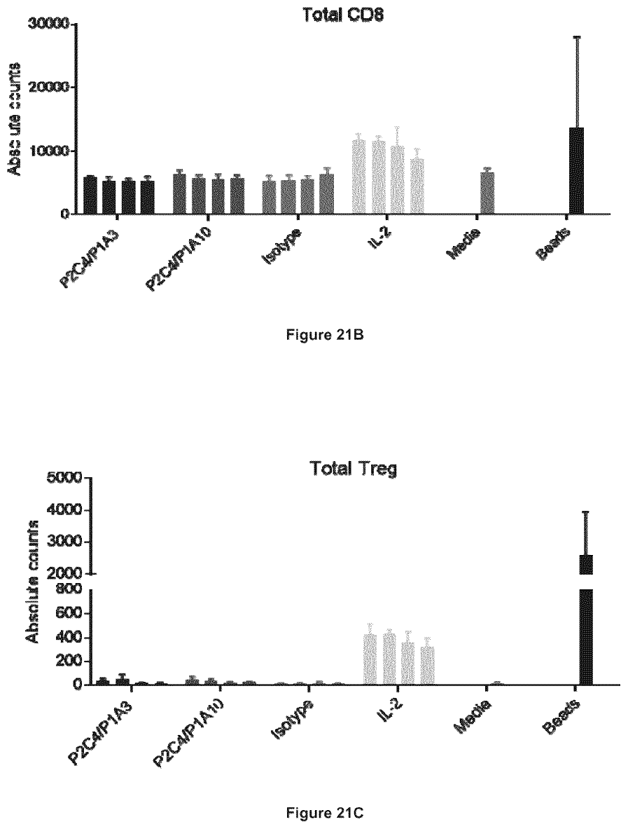

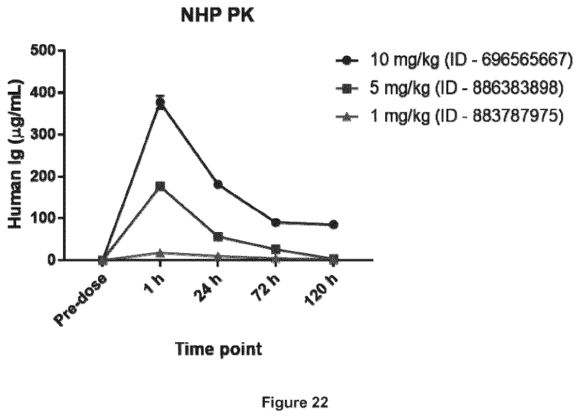

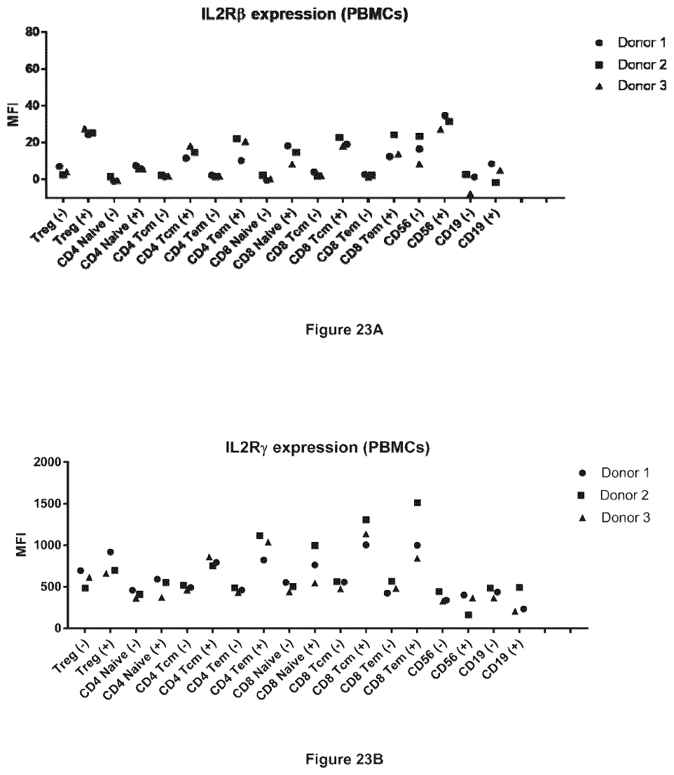

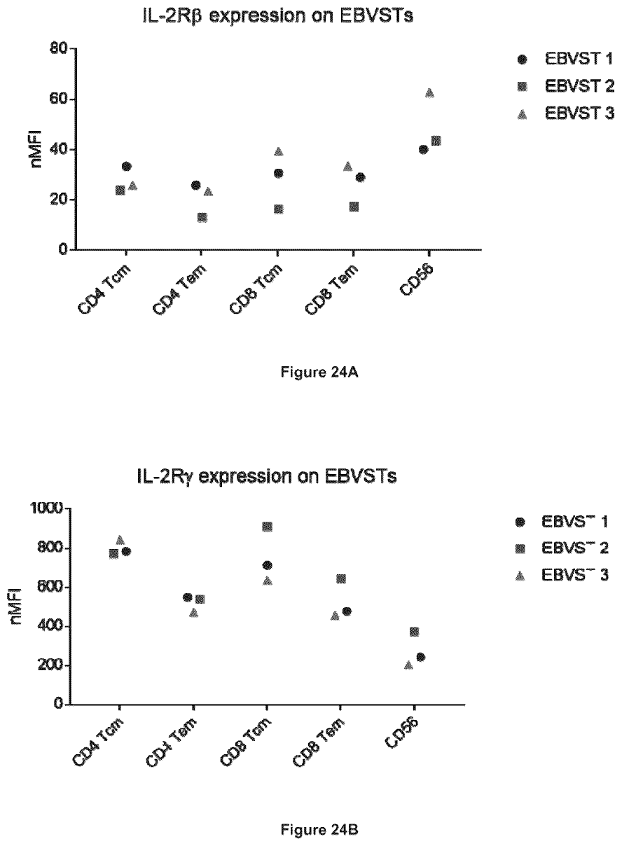

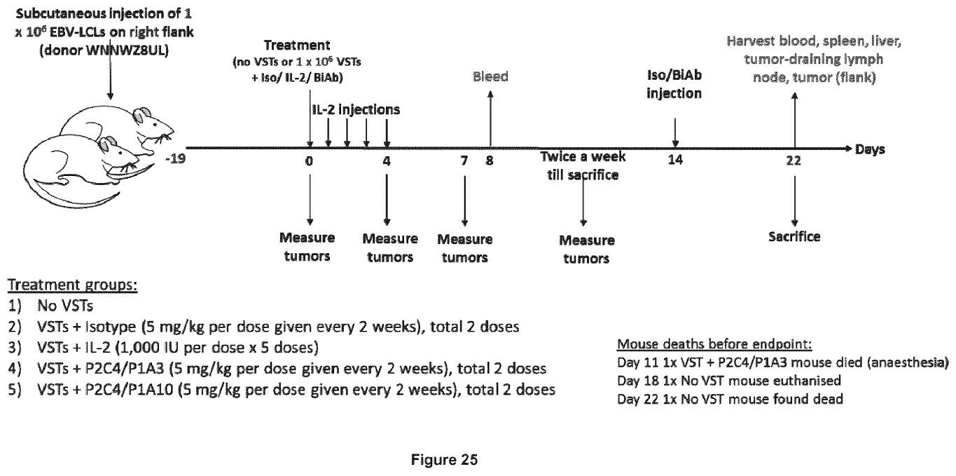

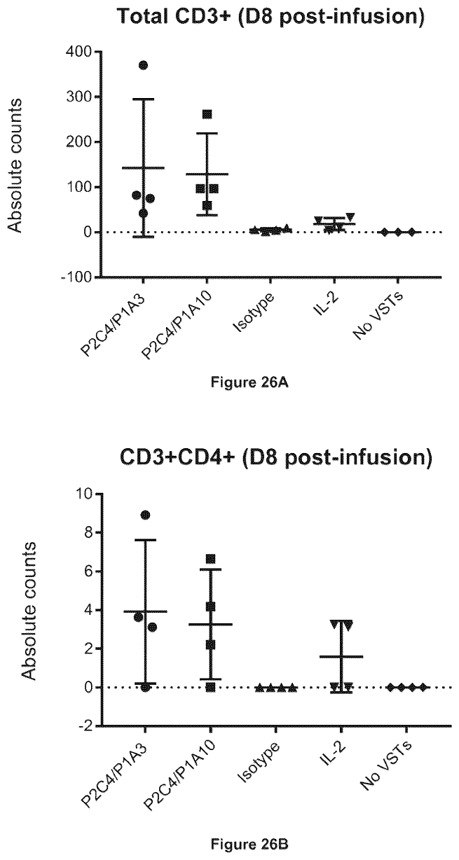

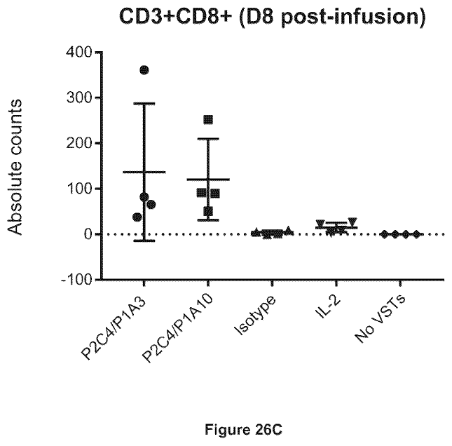

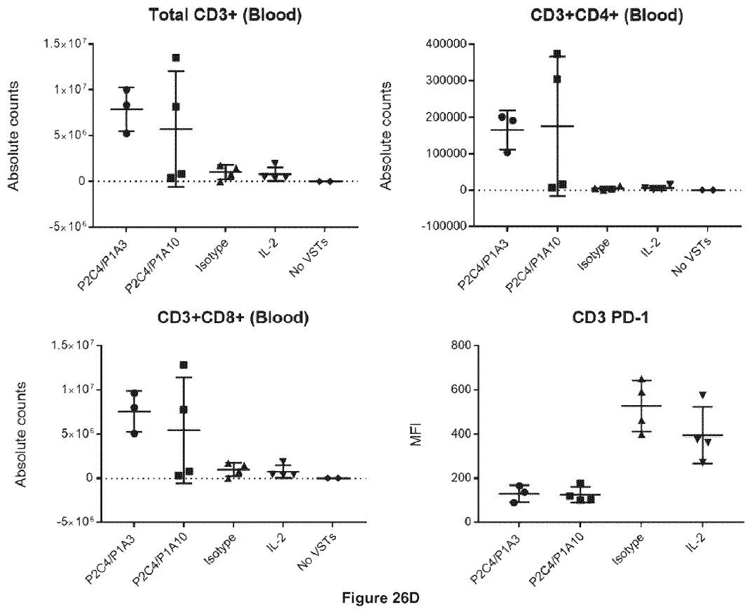

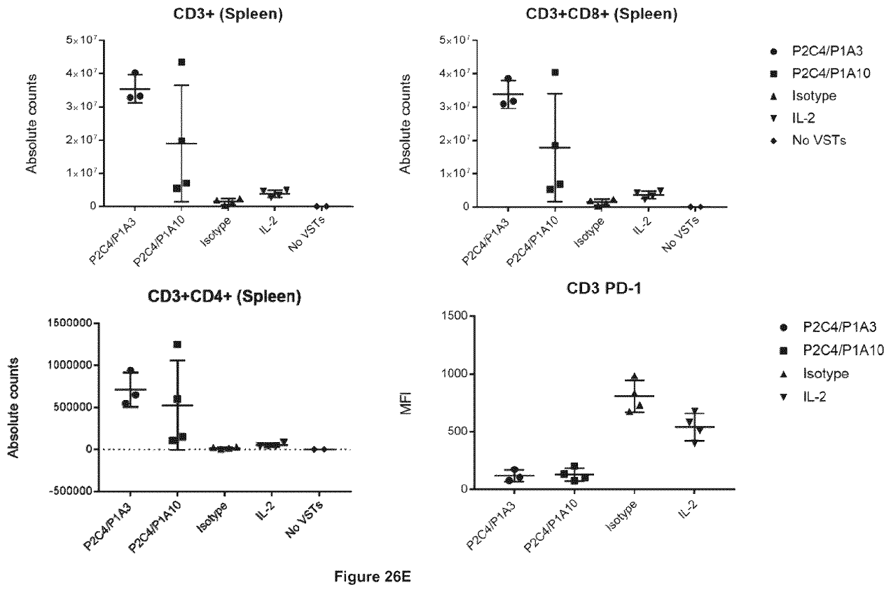

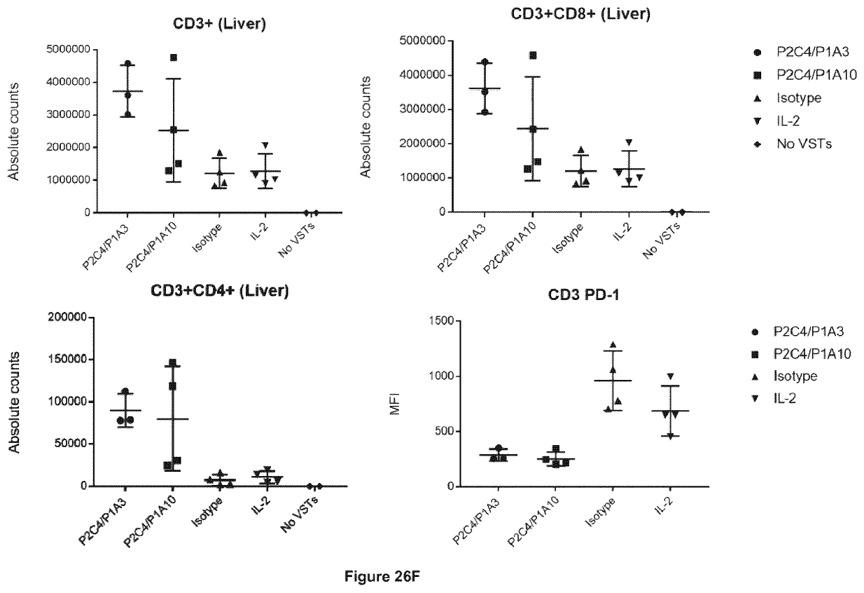

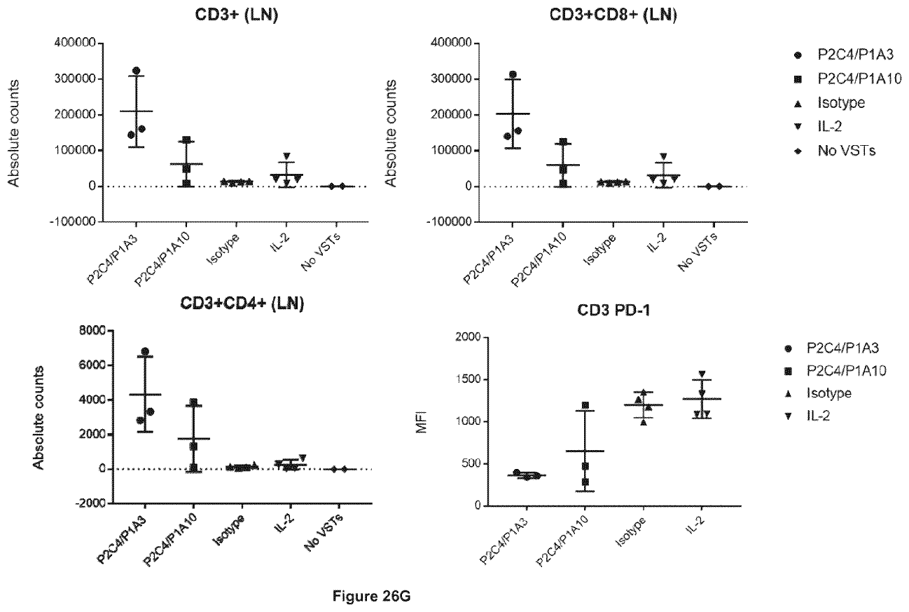

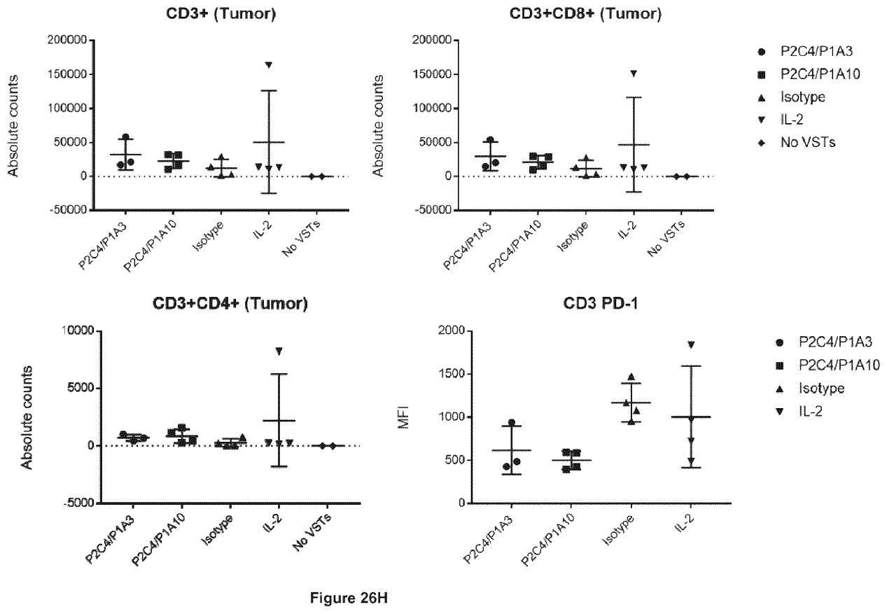

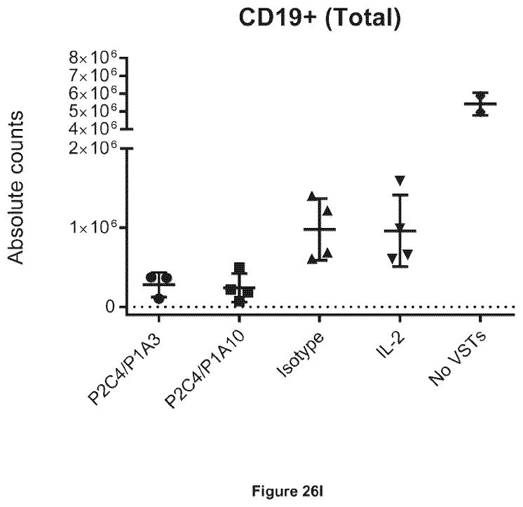

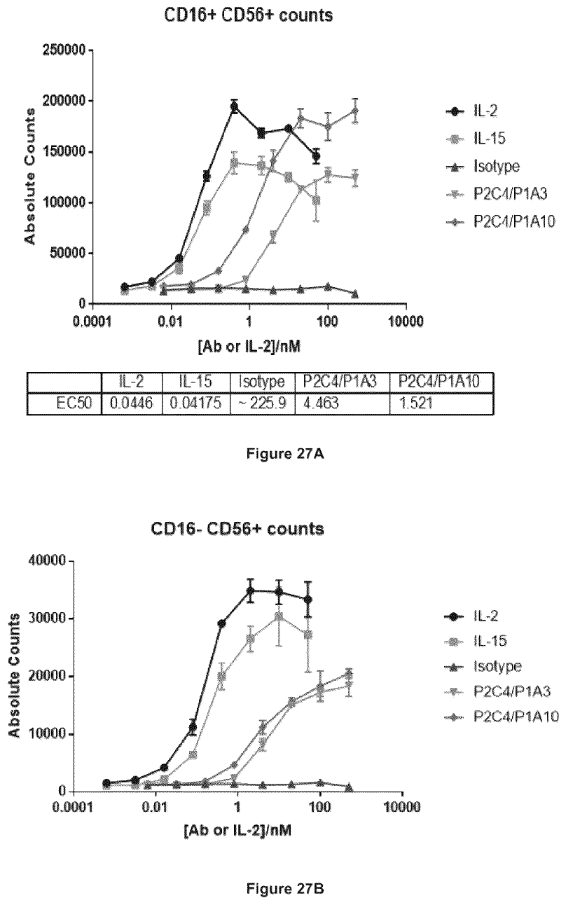

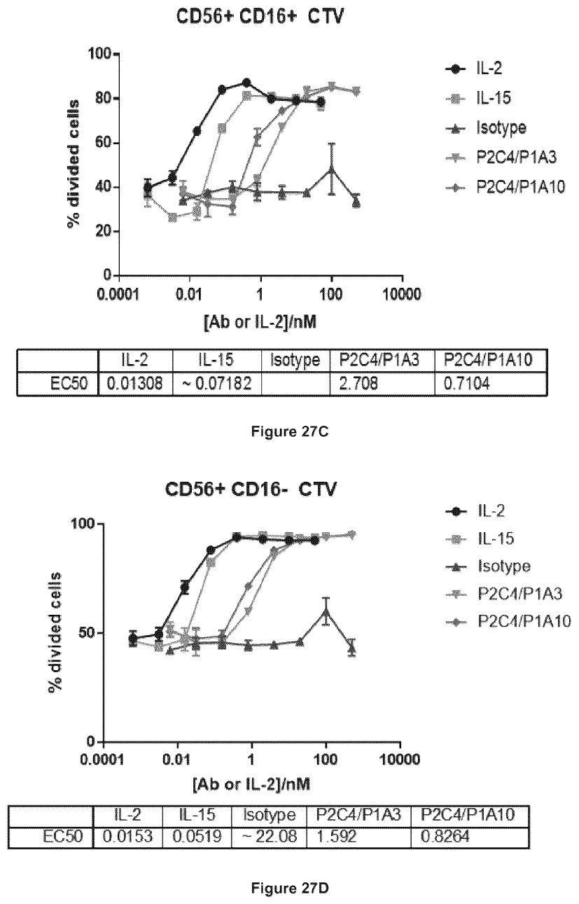

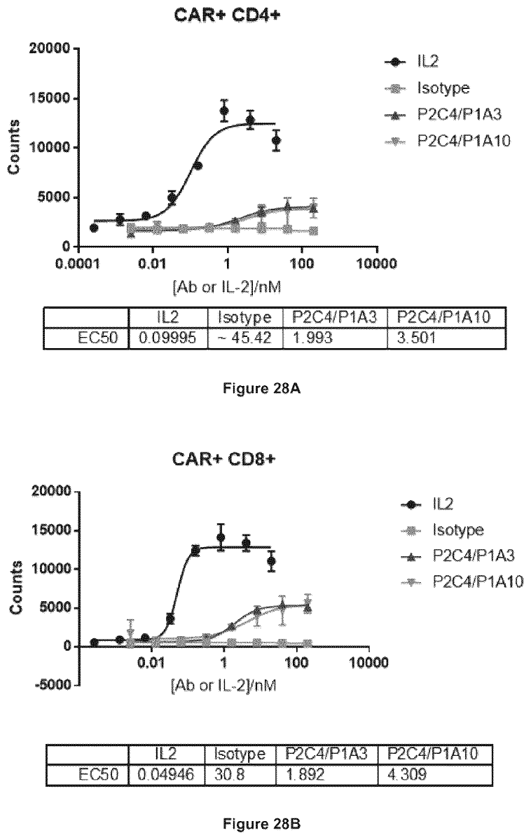

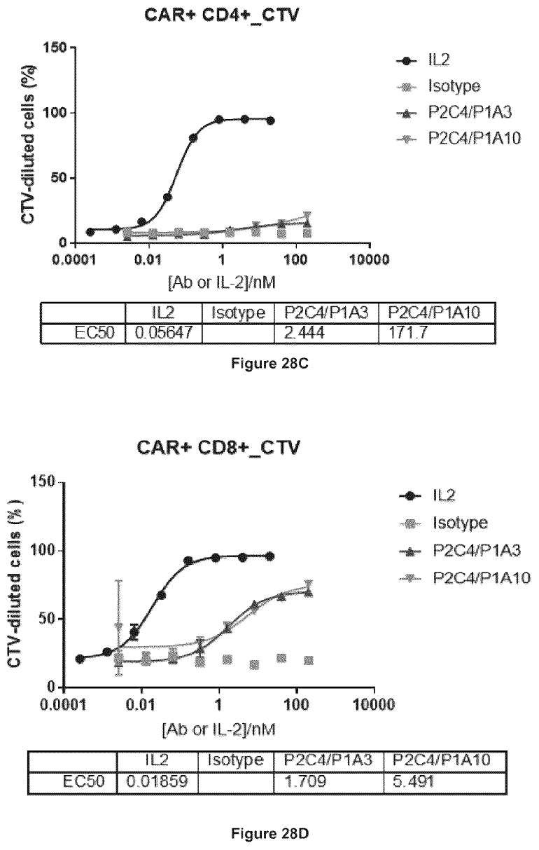

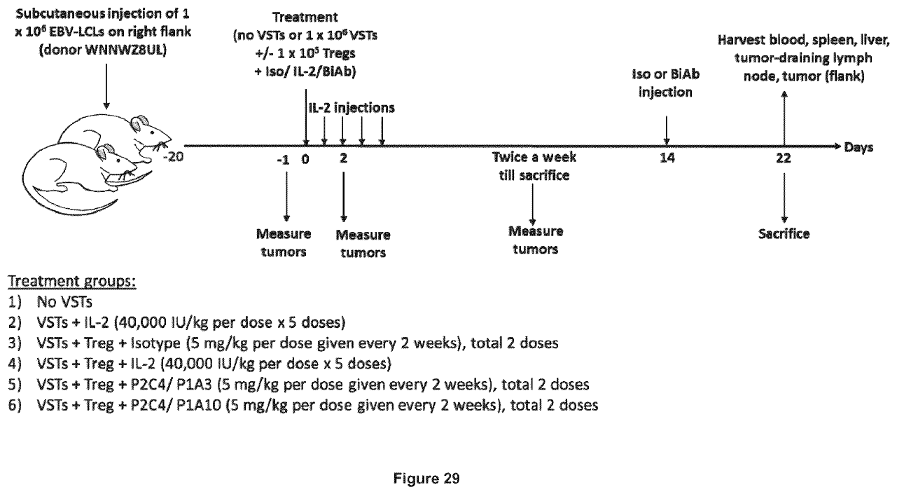

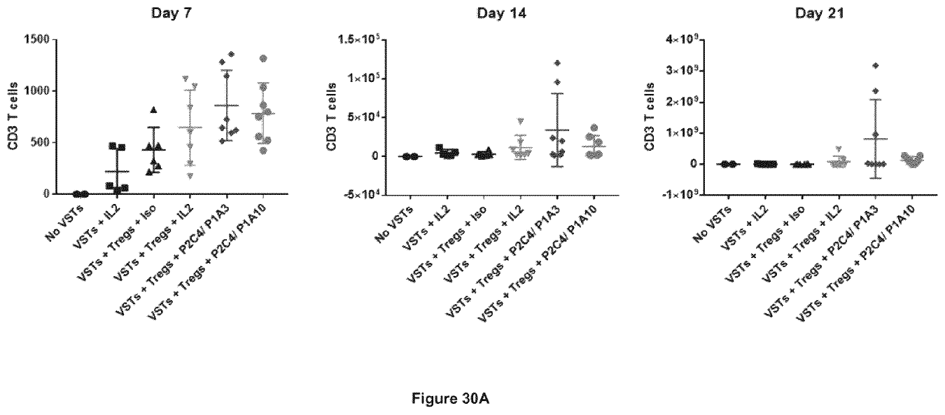

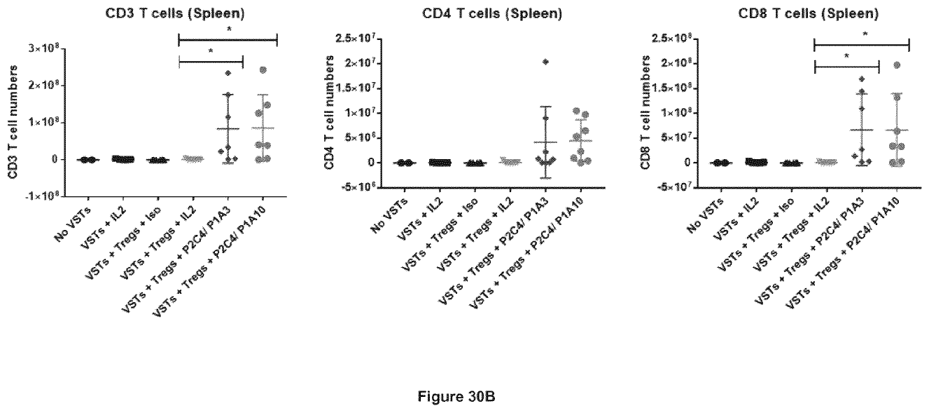

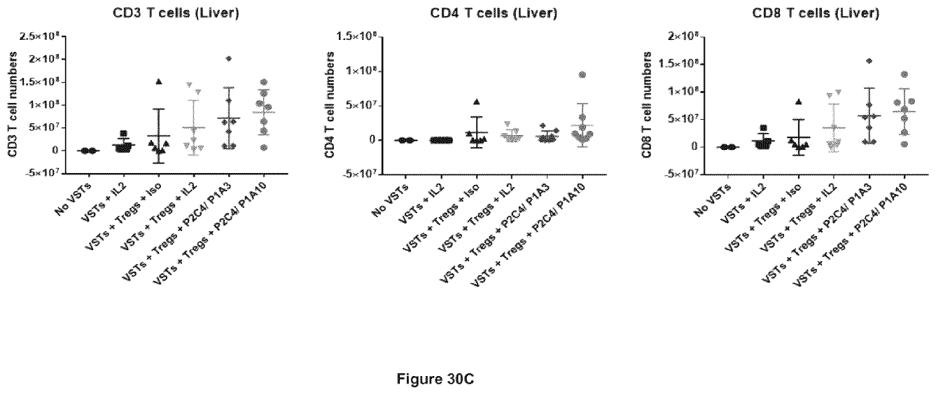

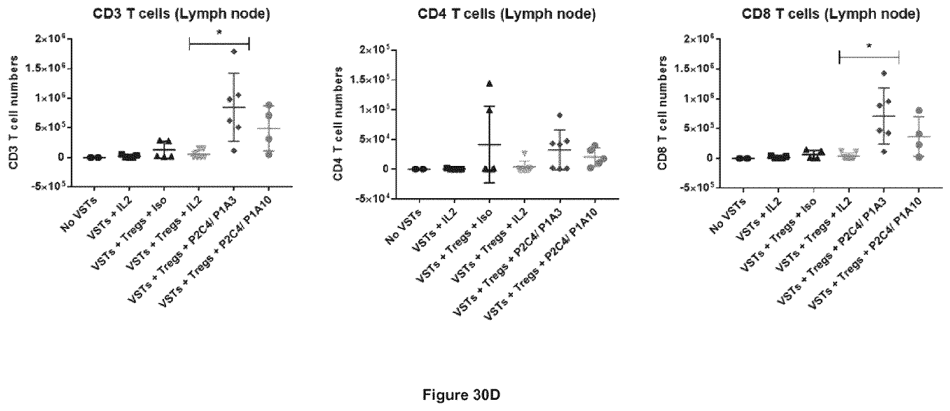

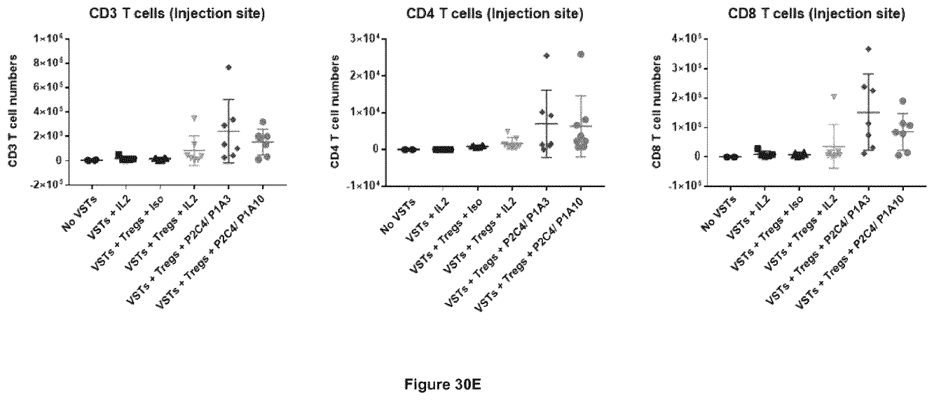

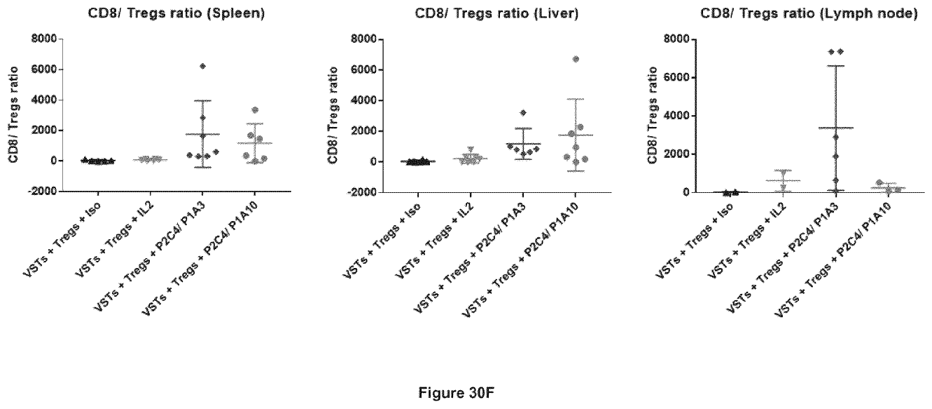

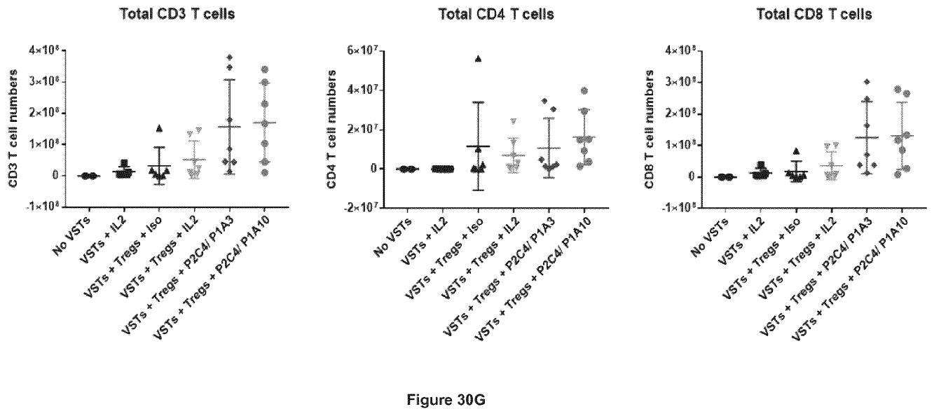

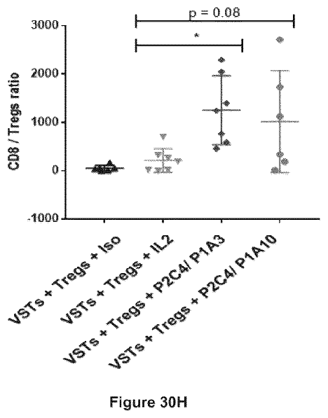

FIGS. 18A to 18C. Graphs showing kinetics of induction of STAT5 phosphorylation in antigen-specific T cells following treatment of with bispecific IL-2Rβ- and γc-binding antibodies or IL-2. (18A) Percentage pSTAT5-positive EBV-specific T cells. (18B) Percentage pSTAT5-positive CD8+EBV-specific T cells. (18C) Percentage pSTAT5-positive CD4+ EBV-specific T cells.

FIG. 19. Western blot showing induction of STAT6 phosphorylation by IL-4 in THP-1 cells following treatment with bispecific IL-2Rβ- and γc-binding antibodies, isotype control antibody, or IL-2. Total STAT6 and actin were included as controls.

FIGS. 20A to 20K. Bar charts showing analysis of proliferation of immune cell subsets in response to treatment of freshly-obtained, non-activated human PBMCs with bispecific IL-2Rβ- and γc-binding antibodies or IL-2. Unstimulated cells (media) and anti-CD3/CD28 bead-stimulated controls (beads) are indicated. (20A) Absolute numbers of CD4+ T cells. (20B) Absolute numbers of CD8+ T cells. (20C) Absolute numbers of Tregs. (20D) Absolute numbers of NK cells. (20E) Absolute numbers of B cells. (20F) Absolute numbers of naïve CD4+ T cells. (20G) Absolute numbers of naïve CD8+ T cells. (20H) Absolute numbers of central memory CD4+ T cells. (20I) Absolute numbers of central memory CD8+ T cells. (20J) Absolute numbers of effector memory CD4+ T cells. (20K) Absolute numbers of effector memory CD8+ T cells.

FIGS. 21A to 21C. Bar charts showing analysis of proliferation of immune cell subsets in response to treatment of non-activated human T cells with bispecific IL-2Rβ- and γc-binding antibodies or IL-2 Unstimulated cells (media) and anti-CD3/CD28 bead-stimulated controls (beads) are indicated. (21A) Absolute numbers of CD4+ T cells. (21B) Absolute numbers of CD8+ T cells. (21C) Absolute numbers of Tregs.

FIG. 22. Graph showing levels of bispecific IL-2Rβ/γc antibody (P2C4:P1A3) in the serum of cynomolgus macaques at the indicated time point, following administration of the indicated amount of antibody, as determined by ELISA.

FIGS. 23A and 23B. Graphs showing expression of (23A) IL-2Rβ and (23B) γc on human immune cell subsets with or without activation using anti-CD3/CD28. The graphs show normalized median fluorescence Intensity (nMFI) of antibody staining for IL-2AB and γc as determined by flow cytometry.

FIGS. 24A and 24B. Graphs showing expression of (24A) IL-2Rβ and (24B) γc on EBV-specific immune cell subsets. The graphs show normalized median fluorescence intensity (nMFI) of antibody staining for IL-2Rβ and γc as determined by flow cytometry.

FIG. 25. Schedule of administration of VSTs with or without bispecific IL-2Rβ- and γc-binding antibodies (BiAb), isotype control antibody or IL-2 to murine EBV-LCL tumour model.

FIGS. 26A to 26I. Graphs showing analysis of proliferation of T cell subsets and PD-1 expression in an in vivo murine EBV-LCL tumour model following treatment with VSTs and bispecific IL-2Rβ- and γc-binding antibodies, isotype control antibody, or IL-2. (26A) Absolute numbers of CD3+ T cells at 8 days post-VST treatment. (26B) Absolute numbers of CD3-CD4+ T cells at 8 days post-VST treatment. (26C) Absolute numbers of CD3+CD8+ T cells at 8 days post-VST treatment. (26D) Absolute numbers of CD3+, CD3+CD4+ and CD3+CD8+ T cells, and CD3 T cell PD-1 expression analysed by MFI, from blood at 22 days post-VST treatment. (26E) Absolute numbers of CD3+, CD3+CD4+ and CD3+CD8+ T cells, and CD3 T cell PD-1 expression analysed by MFI, from the spleen at 22 days post-VST treatment. (26F) Absolute numbers of CD3+, CD3+CD4+ and CD3+CD8+ T cells, and CD3 T cell PD-1 expression analysed by MFI, from the liver at 22 days post-VST treatment. (26G) Absolute numbers of CD3+, CD3+CD4+ and CD3+CD8+ T cells, and CD3 T cell PD-1 expression analysed by MFI, from tumour-draining lymph nodes at 22 days post-VST treatment. (26H) Absolute numbers of CD3+. CD3+CD4+ and CD3+CD8+ T cells, and CD3 T cell PD-1 expression analysed by MFI, from tumour at 22 days post-VST treatment. (26I) Total organ tumour load shown by the absolute total number of CD19+ cells in spleen, liver, tumour-draining lymph node and tumour.

FIGS. 27A to 27D. Graphs showing analysis of proliferation of pre-activated human NK cells following treatment with different amounts of bispecific IL-2Rβ- and γc-binding antibodies or the indicated cytokines. Isotype antibody was used as a control. (27A) Absolute numbers of CD16+CD56+ NK cells. EC50 values are shown. (27B) Absolute numbers of CD16-CDS6+ NK cells. (27C) Percentage of dividing cells that are CD16+CD66+, determined by CellTrace Violet (CTV). EC50 values are shown. (27D) Percentage of dividing cells that are CD16-CD56+, determined by CTV. EC50 values are shown.

FIGS. 28A to 28D. Graphs showing analysis of proliferation of CAR-T cells following treatment with different amounts of bispecific IL-2Rβ- and γc-binding antibodies or IL-2. Isotype antibody was used as a control. EC50 values are shown for 28A-D. (28A) Absolute numbers of CD4+ CAR-T cells. (28B) Absolute numbers of CD4+ CAR-T cells. (28C) Percentage of dividing CAR-T cells that are CD4+, determined by CellTrace™ Violet (CTV). (28D) Percentage of dividing CAR-T cells that are CD8+, determined by CTV.

FIG. 29. Schedule of administration of VSTs and Tregs with or without bispecific IL-2Rβ- and γc-binding antibodies (BiAb), isotype control antibody (iso) or IL-2 to murine EBV-BLCL tumour model.

FIGS. 30A to 30K. Graphs showing analysis of proliferation of T cell subsets in an in vivo murine EBV-BLCL tumour model following treatment with VSTs and Tregs plus bispecific IL-2Rβ- and γc-binding antibodies, isotype control antibody (Iso), or IL-2. (30A) Absolute numbers of CD3+ T cells at 7, 14 and 21 days post-treatment. (30B) Absolute numbers of CD3+, CD3+CD4+ and CD3+CD8+ T cells from the spleen at 22 days post-treatment. (30C) Absolute numbers of CD3+, CD3+CD4+ and CD3+CD8+ T cells from the liver at 22 days post-treatment. (30D) Absolute numbers of CD3+, CD3+CD4+ and CD3+CD8+ T cells from tumour-draining lymph node at 22 days post-treatment. (30E) Absolute numbers of CD3+, CD3+CD4+ and CD3+CD8+ T cells from injection site at 22 days post-treatment. (30F) Ratio of the absolute number of CD8+ T cells to the absolute number of Tregs in spleen, liver and tumour-draining lymph node at 22 days post-treatment. (30G) Total number of CD3+, CD3+CD4+ and CD3+CD8+ T cells pooled from spleen, liver, tumour-draining lymph node and injection site. (30H) Ratio of the total number of CD8+ T cells to the total number of Tregs pooled from spleen, liver, tumour-draining lymph node, and injection site at 22 days post-treatment. (30I) Absolute numbers of EBV-BLCL tumour cells from spleen, liver and lymph node(s) at 22 days post-treatment. (30J) Total organ tumour load shown by the absolute total number of CD19+ tumour cells in spleen, liver, and tumour-draining lymph node. (30K) Total number of CD107a+. IFN-γ+ and perforin+ CD8 T cells pooled from spleen, liver, tumour-draining lymph node, and injection site at 22 days post-treatment.

### EXAMPLES

In the following Examples, the inventors design, produce and characterise antibodies capable of binding to IL-2Rβ and γc.

### Example 1: IL-2Rβ and γc Binding Antibodies

Anti-IL-2Rβ antibody clones and anti-γc antibody clones were isolated from a human antibody phage display library via in vitro selection.

Illustrative bispecific antibodies were constructed using IL-2Rβ-binding clone P2C4 in combination with one of the γc-binding antibody clones P1A3 or P1A10. The bispecific antibodies were designated ‘P2C4/P1A3’ and ‘P2C4/P1A10’, respectively.

The closest matching antibody germline genes for clone P2C4 are IGHV1-46*01 and IGLV2-14*01.

The closest matching antibody germline genes for clone P1A3 are IGHV4-34*01 and IGKV2-28*01.

The closest matching antibody germline genes for clone P1A10 are IGHV1-24*01 and IGKV2-28*01.

Three bispecific antibody formats were prepared: scFv-KIH-Fc, CrossMab and Duobody formats. The bispecific antibodies were expressed by transient transfection of HEK 293 cells, and yields were as follows:
ScFv-KiH-Fc:
   P2C4/P1A3: 4-14 mg/L; P2C4/P1A10: 28-40 mg/L
CrossMab:
   P2C4/P1A3: 14-160 mg/L; P2C4/PIA 10: 63 mg/L
Duobody:
   P2C4/P1A10: (P2C4) 77 mg/L; (P1A10) 110 mg/L

Except where otherwise indicated, in the following examples P2C4/P1A3 and P2C4/P1A10 were investigated in the scFv-KiH-Fc format, in which scFv comprising VH and VL domains for P2C4 are fused via a linker to Fc comprising the ‘knob’ modification is expressed with scFv comprising VH and VL domains for P1A3 (P2C4/P1A3) or P1A10 (P2C4/P1A10) fused via a linker to Fc comprising the ‘hole’ modification.

### Example 2: Analysis of Binding to IL-2 Receptors

2.1 Analysis of Binding Affinity by ELISA

Binding of P2C4/P1A3 to IL-2Rβ or γc was measured by ELISA analysis, using recombinant IL-2Rβ-Fc and γc-Fc coated on maxisorp plates.

Biotinylated P2C4/P1A3 was added at various concentrations. Detection of binding was performed using a colorimetric assay using HRP-conjugated streptavidin which converts TMB substrate to a blue solution. The reaction was stopped using hydrochloric acid, and absorbance was measured at 450 nm and 670 nm.

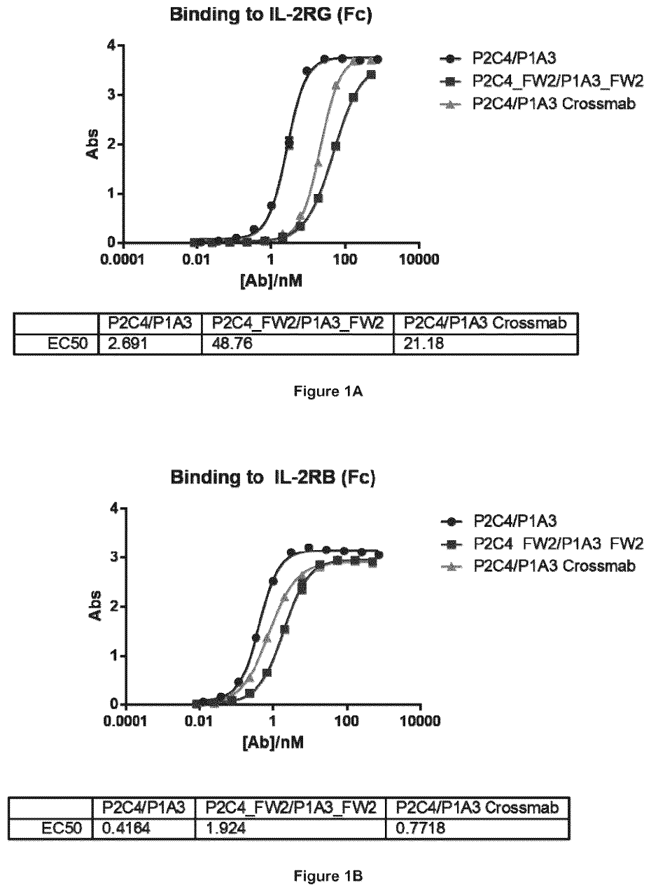

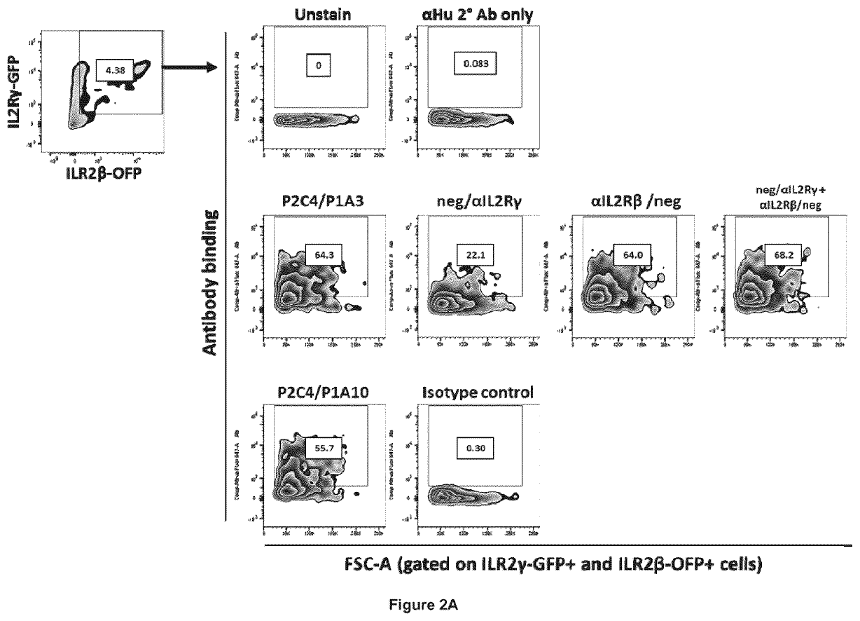

The results are shown in FIGS. 1A and 1B. P2C4/P1A3 was shown to bind to both IL-2Rβ and γc. EC50s for binding were calculated and are shown in the Figures.

The bispecific antibodies analysed in this assay were:
scFv (P2CA) scFv (P1A3)—KiH-Fc—designated P2C4/P1A3′ in the Figures.
scFv (P2C4_FW2); scFv (P1A3_FW2)—KIH-Fc—designated P2C4_FW2/P1A3_ FW2′ in the Figures.
Fab (P2C4): Fab (P1A3) in CrossMab format—designated ‘P2C4/P1A3 Crossmab’ in the Figures.

2.2 Analysis of Binding Affinity by Bio-Layer Interferometry

The affinity of binding of P2C4/P1A3 and P2C4/P1A10 to IL-2Rβ and γc was measured by Bio-Layer Interferometry (BLI).

P2C4/P1A3 or P2C4/P1A10 were captured on anti-human Fc biosensor tips, and 5 different concentrations of monomeric IL-2Rβ or γc were allowed to bind to the captured antibodies. Dissociation of the antigen from the immobilised antibodies was performed for 5 min Binding affinity was calculated by fitting binding curves using the 1:1 Langmuir model.

The affinity data are summarised in the table below.

| | IL -2Rβ | | γc | |
|---|---|---|---|---|
| P2C4/P1A3 | kₒₙ = 2.21 × 10⁵ M¹s¹ | k_{off} = 6.62 × 10³ s¹ | kₒₙ = 6.22 × 10⁴ M¹s¹ | k_{off} = 4.42 × 10³ s¹ |
| | Ko = 3.00 × 10⁸ M | | Ko = 8.47 × 10⁸ M | |
| P2C4/P1A10 | kₒₙ = 1.66 × 10⁵ M¹s¹ | k_{off} = 4.40 × 10³ s¹ | k_{off} = 1.56 × 10⁵ M¹s¹ | k_{off} = 9.61 × 10³ s¹ |

| | |
|---|---|
| Ko = 2.82 × 10⁸ M | Ko = 6.18 × 10⁸ M |

Similar binding to IL-2Rβ for P2C4/P1A3 and P2C4/P1A10 was observed (30 nM vs 28.2 nM). This was to be expected because the bispecific antibodies have the same IL-2Rβ-binding clone P2C4.

Whilst the affinity of binding to γc was similar for P2C4/P1A3 and P2C4/P1A10 (84.7 nM vs 61.8 nM). P2C4/P1A10 was found to have a faster on-rate and a faster off-rate than P2C4/P1A3.

2.3 Analysis of Binding to IL-2RD and Yc Expressed at the Cell Surface

To determine whether P2C4/P1A3 and P2C4/P1A10 are able to bind to IL-2 receptors expressed on the surface of cells, HEK293-6E cells were transfected with plasmids encoding human IL-2Rα-GFP, or IL-2Rβ-OFP and γc-GFP.

Transfected cells were stained with P2C4/P1A3, P2C4/P1A10 or an isotype control antibody, followed by detection with a fluorochrome-conjugated secondary antibody for analysis by flow cytometry.

Normalized Median Fluorescence Intensity (nMFI) was calculated in the GFP+ cell population (for calls transfected with constructs encoding IL-2Rα-GFP) or the GFP+/OFP+ cell population (for cells transfected with constructs encoding IL-2Rβ-OFP and γc-GFP) by subtracting the MFI obtained when secondary antibody only was added to the cells (negative control condition).

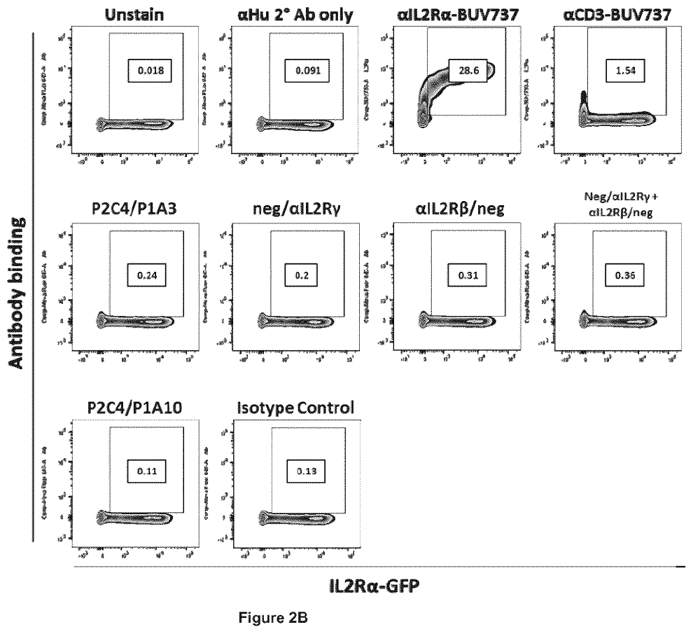

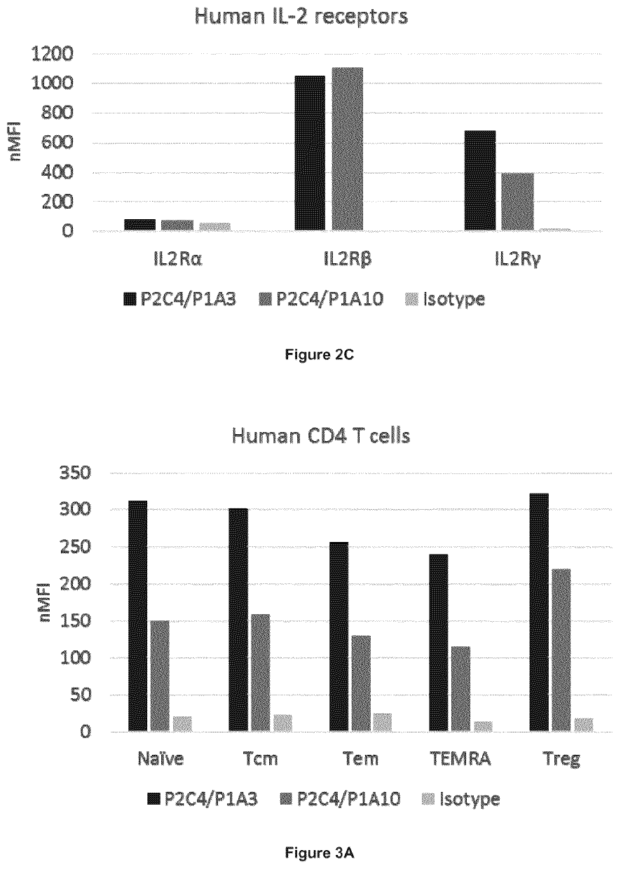

The results of the analysis are shown in FIGS. 2A to 2C. Both P2C4/P1A3 and P2C4/P1A10 showed specific binding to cells expressing human IL-2Rβ and γc, but did not bind to cells expressing IL-2Ra.

2.4 Analysis of Binding to Human T Cell Subsets

To identify the subsets of human T cells that P2C4/P1A3 and P2C4/P1A10 bind to, human peripheral blood mononuclear cells (PBMCs) were isolated and stained with P2C4/P1A3, P2C4/P1A10 or isotype control antibody, followed by detection with a fluorochrome-conjugated secondary antibody. Cells were then stained with antibodies for the T cell markers CD3, CD4, CD8, CD45RA, CCR7, Foxp3 and CD25 to enable the delineation of the following T cell subsets: Naive (CD45RA+CCR?+). T central memory (CD45RA-CCR7+), T effector memory (CD45RA-CDR7−), T effector memory re-expressing CD45RA (TEMRA: CD45RA+CCR7−) and Treg (CD4+CD25+Foxp3+).

Samples were analysed by flow cytometry. Normalized Median Fluorescence Intensity (nMFI) was calculated by subtracting the MFI of the secondary antibody control.

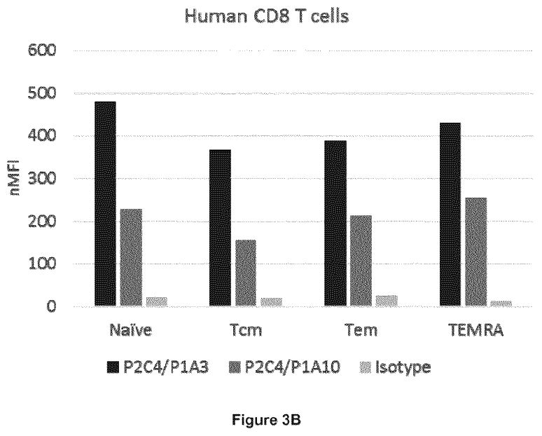

The results are shown in FIGS. 3A and 3B. P2CA/P1A3 and P2C4/P1A10 were found to bind to all of the different human T cell subsets tested. P2C4/P1A10 displayed reduced level of binding as compared to P2C4/P1A3.

2.5 Analysis of Binding to Rhesus IL-2Rβ and γc Expressed at the Cell Surface

Cross-reactivity of P2C4/P1A3 and P2C4/P1A10 for rhesus IL-2Rβ and γc was analysed essentially as described in Example 2.3 above, using HEK293-6E cells transfected with plasmids encoding rhesus IL-2Rβ-OFP and γc-GFP.

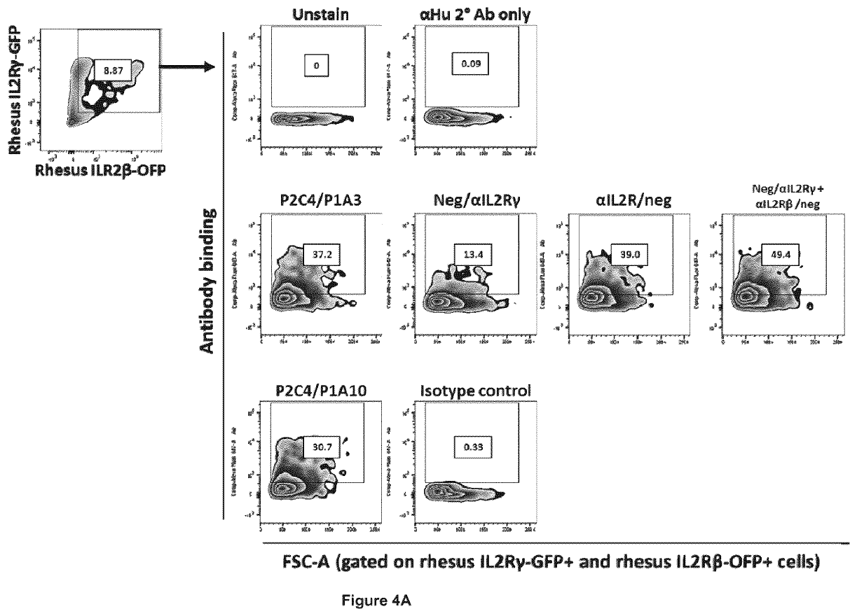

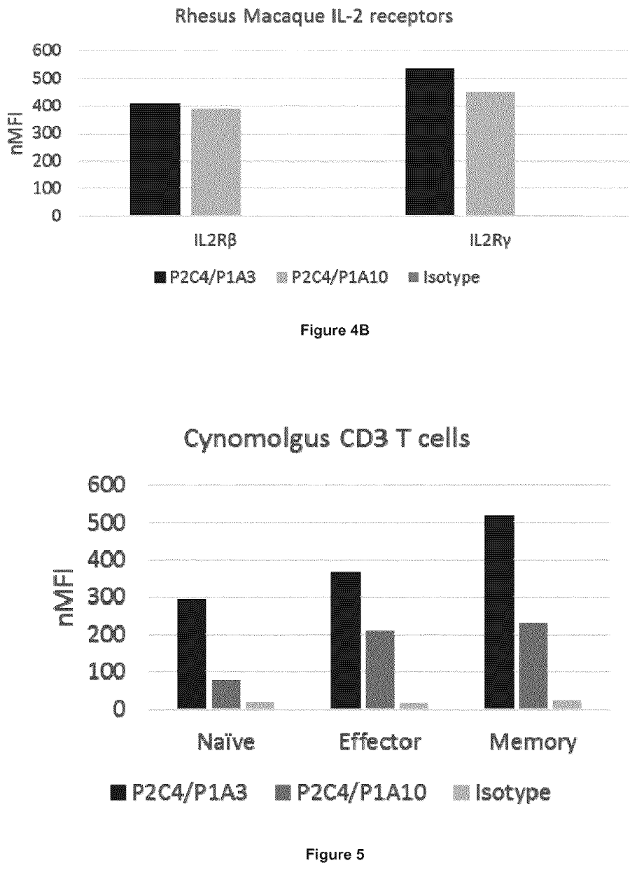

The results of the analysis are shown in FIGS. 4A and 4B. Both P2C4/P1A3 and P2C4/P1A10 showed specific binding to cells expressing rhesus IL-2Rβ and γc.

2.6 Analysis of Binding to Cynomolgus Macaque T Cells

Cynomolgus macaque PBMCs were isolated and stained with P2C4/P1A3, P2C4/P1A10 or isotype control antibody, followed by a fluorochrome-conjugated secondary antibody. Cells were then stained with T call markers CD3, CD28 and CD95 to delineate the following T cell subsets: Naive (CD28+CD95−). Effector (CD28-CD95+) and Memory (CD28+CD95+).

Samples were analysed by flow cytometry. Normalized Median Fluorescence Intensity (nMFI) was calculated by subtracting the MFI of the secondary antibody control.

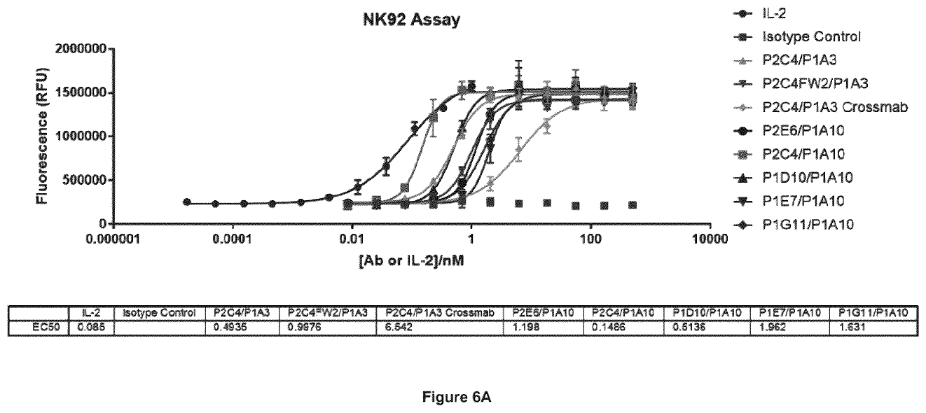

The results are shown in FIG. 5. P2C4/P1A3 and P2C4/P1A10 were found to bind to naïve, effector and memory subsets of cynomolgus T calls P2C4/P1A10 displayed reduced level of binding as compared to P2C4/P1A3.

### Example 3: Analysis of Induction of Cell Proliferation by IL-2R6- and γc-Binding Bispecific Antibodies

3.1 Analysis of the Effect on NK Cells

To analyse the functional activity of the IL-2Rβ- and γc-binding bispecific antibodies, a stimulation assay was performed using the NK92 cell line which expresses both IL-2Rβ and γc.

Anti-IL-2Rβ antibody clones and anti-γc antibody clones identified from human antibody phage display library were paired to form various bispecific antibody combinations, based on a single chain variable fragment (scFv) linked to a IgG1 knob or hole Fc. These antibodies were then used in a NK92 cell stimulation assay.

Briefly, cells were washed and stimulated with antibodies or cytokines for 3 days. Trastuzumab was used as a negative control. To quantify cell proliferation, alamarBlue reagent was added and fluorescence signal was measured at fluorescence excitation wavelength 544 nm and fluorescence emission wavelength 590 nm.

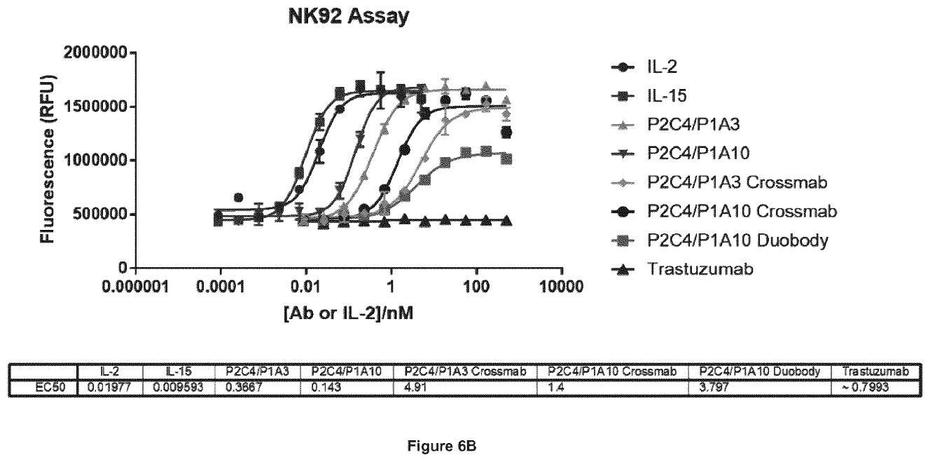

The results are shown in FIG. 6A. Several combinations anti-IL2RB and anti-γc clones are capable of Inducing NK92 cell proliferation.

In a separate assay, the following bispecific antibodies were analysed.
scFv (P2C4): scFv (P1A3)—KiH-Fc—designated P2C4/P1A3′ in the Figure.
scFv (P2C4) scFv (P1A10)—KIH-Fc—designated P2C4/P1A10′ in the Figure.
Fab (P2C4): Fab (P1A3) in CrossMab format—designated ‘P2C4/P1A3 Crossmab’ in the Figure.
Fab (P2C4): Fab (P1A10) in CrossMab format—designated ‘P2C4/P1A10 Crossmab’ in the Figure.
Fab (P2C4): Fab (P1A10) in Duobody format—designated ‘P2C4/P1A10 Duobody’ in the Figure.

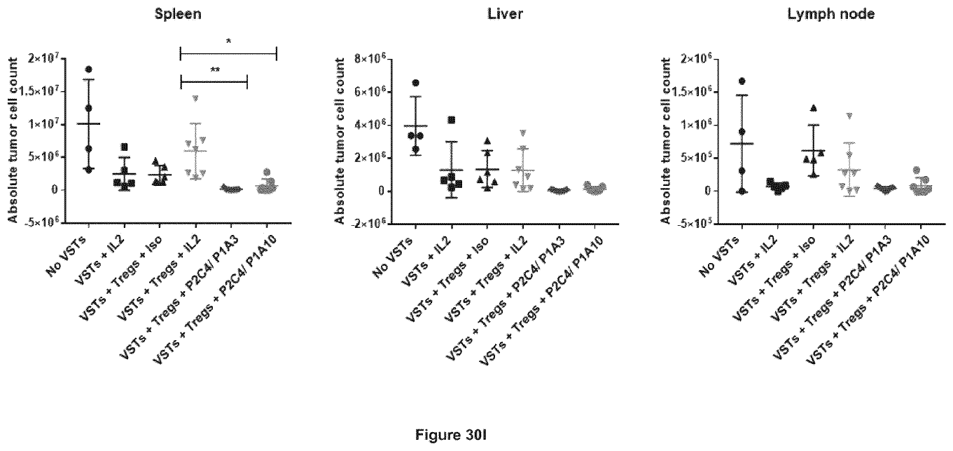

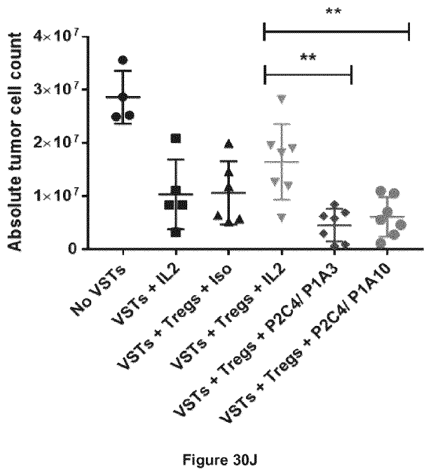

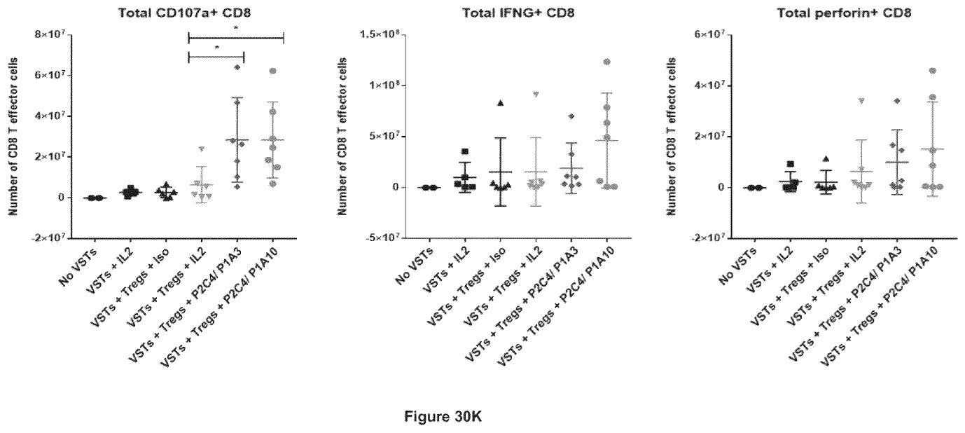

The results are shown in FIG. 68. P2C4/P1A3 and P2C4/P1A10 induced NK92 proliferation in a dose-dependent manner, with an average EC50 of 0.43 nM and 0.16 nM respectively from four independent experiments.

3.2 Analysis of the Effect on Primary Human T Cells

To analyse the effects of P2C4/P1A3 and P2C4/P1A10 on primary human T cells, T cells were isolated from human PBMCs and pre-activated for three days with anti-CD3-coated plates (2 μg/ml) plus soluble anti-CD28 (1 μg/ml). Cells were then rested in fresh media for a day before being labelled with CellTrace Violet, Cells were seeded at 100,000 per well and treated with P2C4/P1A3, P2C4/P1A10 (200 nM, 40 nM, 8 nM and 1.6 nM), IL-2 (20 nM, 4 nM, 0.8 nM, 0.16 nM) or anti-CD3/CD28 beads. Isotype antibody and untreated cells were included as negative controls. After four days, cells were stained with T cell markers CD3, CD4, CD8, CD45RO, CCR7, Foxp3 and CD25 to delineate T cell subsets:
CD4+naïve T cells: CD3+CD4+FoxP3−CCR7+CD45RO−
CD8+naïve T cells: CD3+CD8+CCR7+CD45RO−
CD4+ central memory T cells: CD3+CD4+FoxP3−CCR7+CD45RO+
CD8+ central memory T cells: CD3+CD8+CCR7+CD45RO+
CD4+ effector memory T cells: CD3+CD4+FoxP3−CCR7−CD45RO+
CD8+ effector memory T cells: CD3+CD8+CCR7−CD45RO+
CD4+ Tregs: CD3+CD4+CD25+FoxP3+

Counting beads were included to allow absolute cell numbers to be determined by flow cytometry.

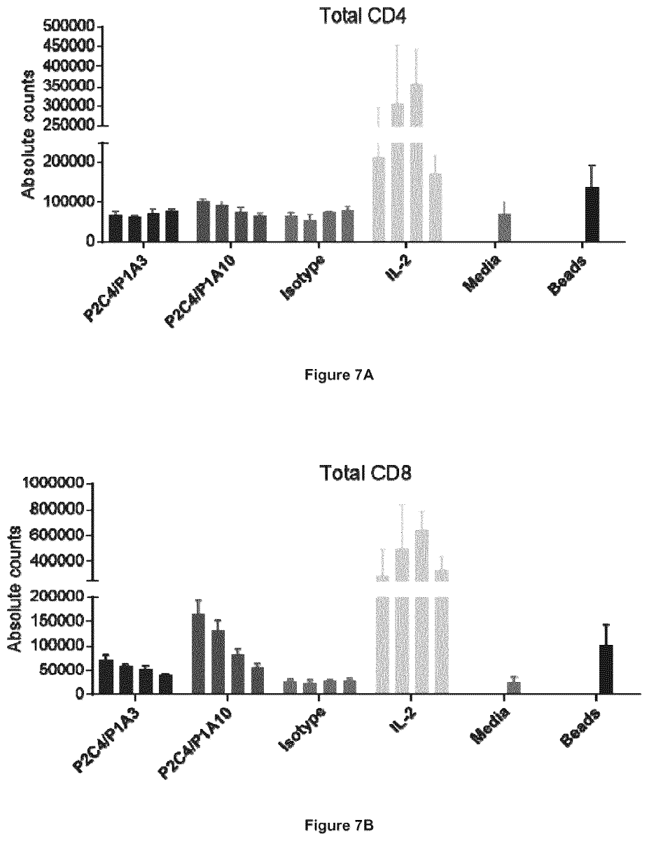

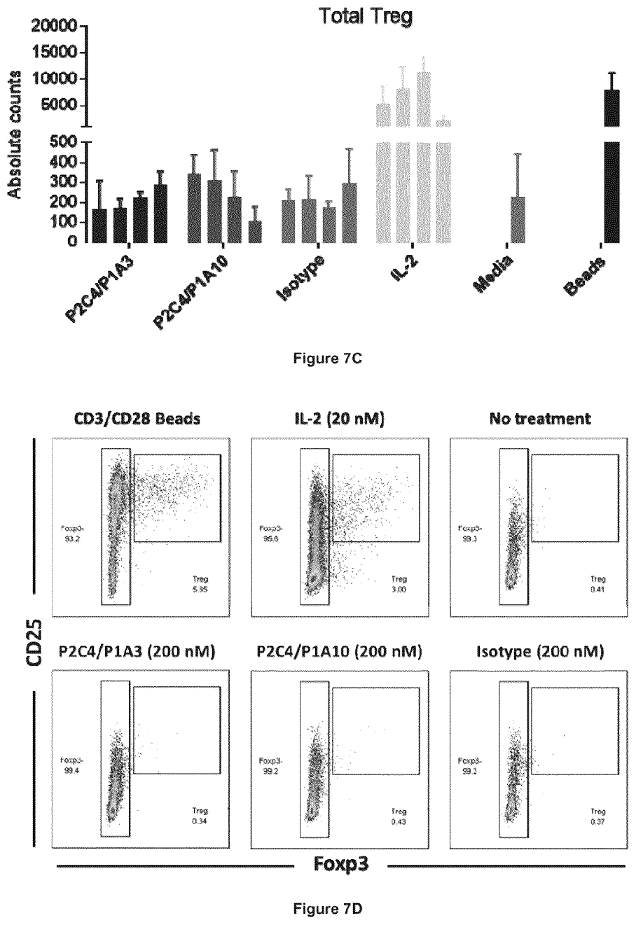

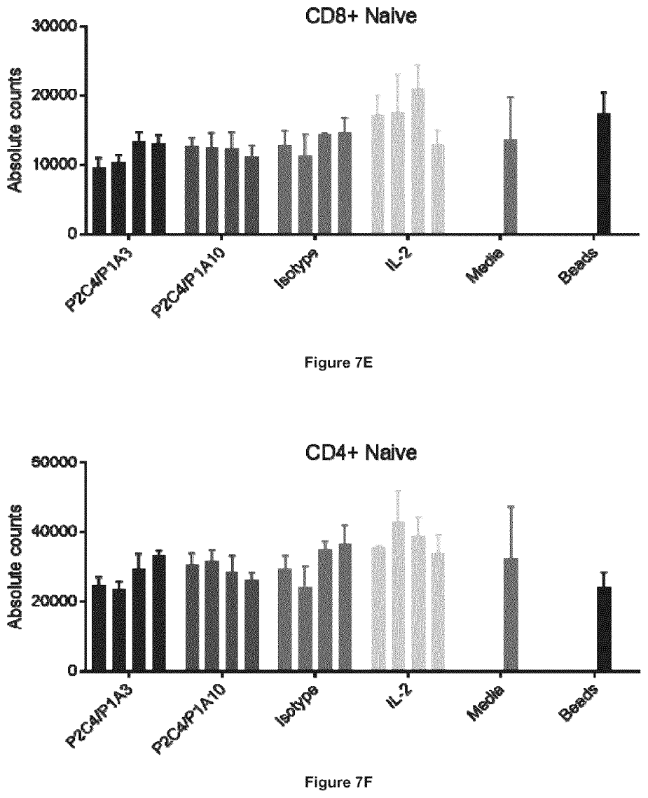

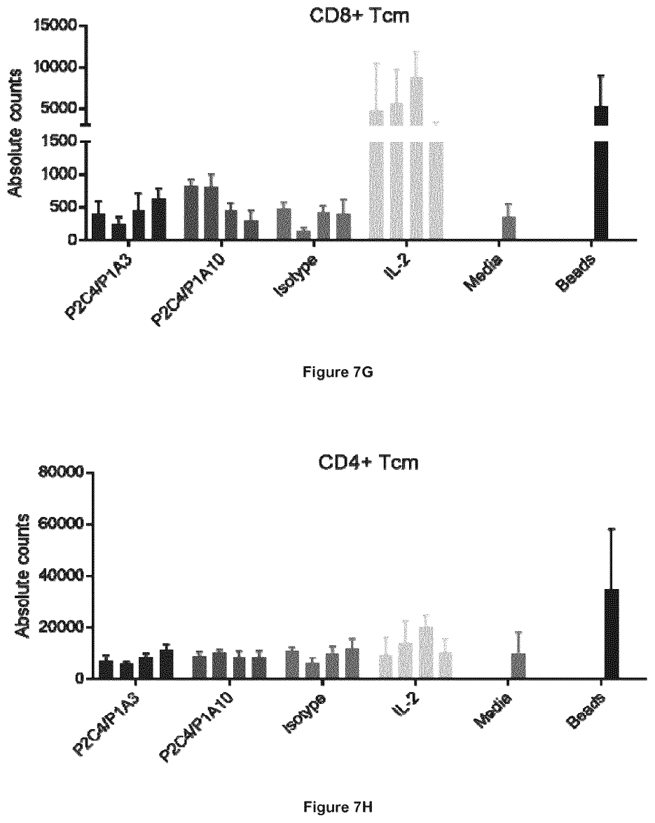

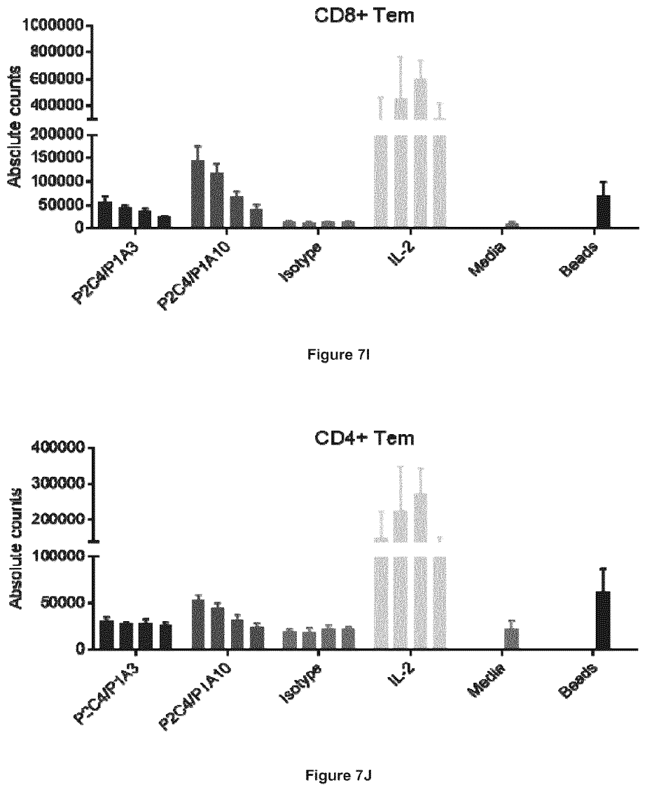

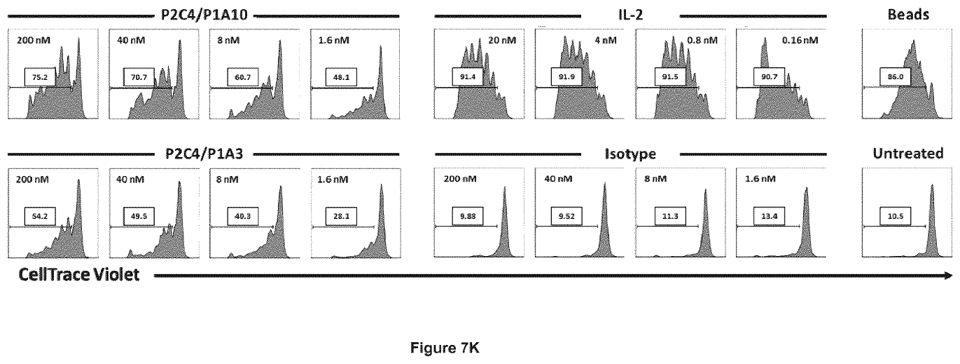

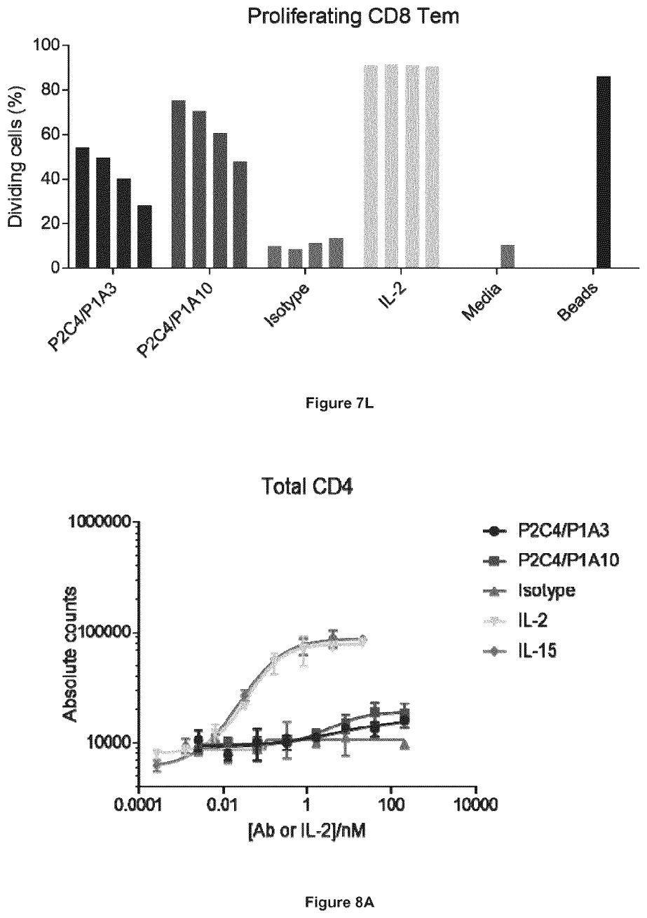

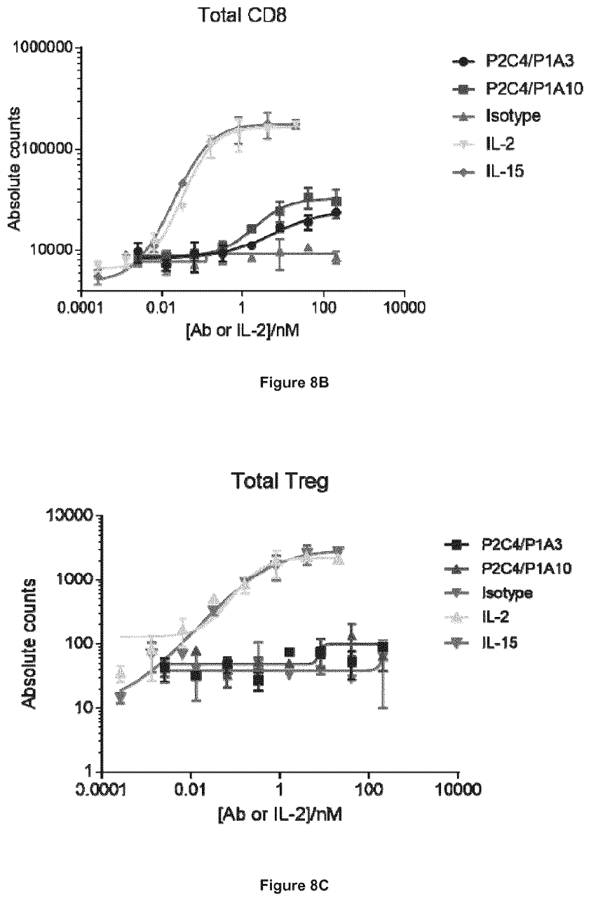

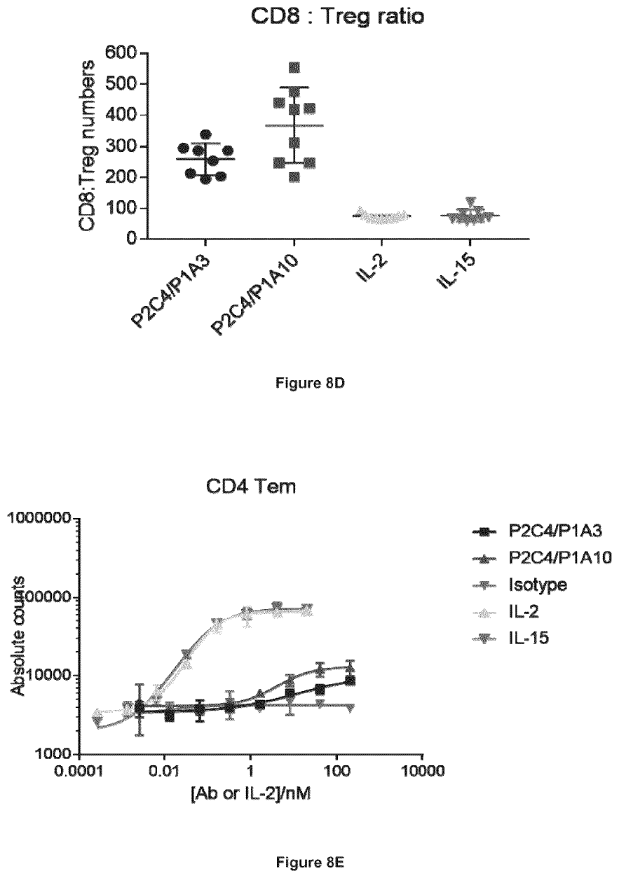

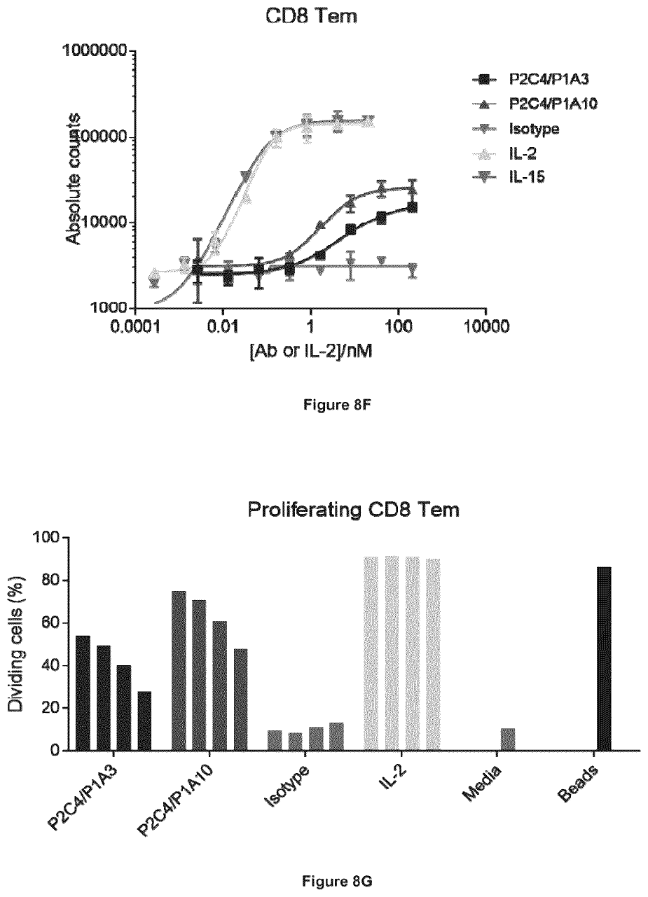

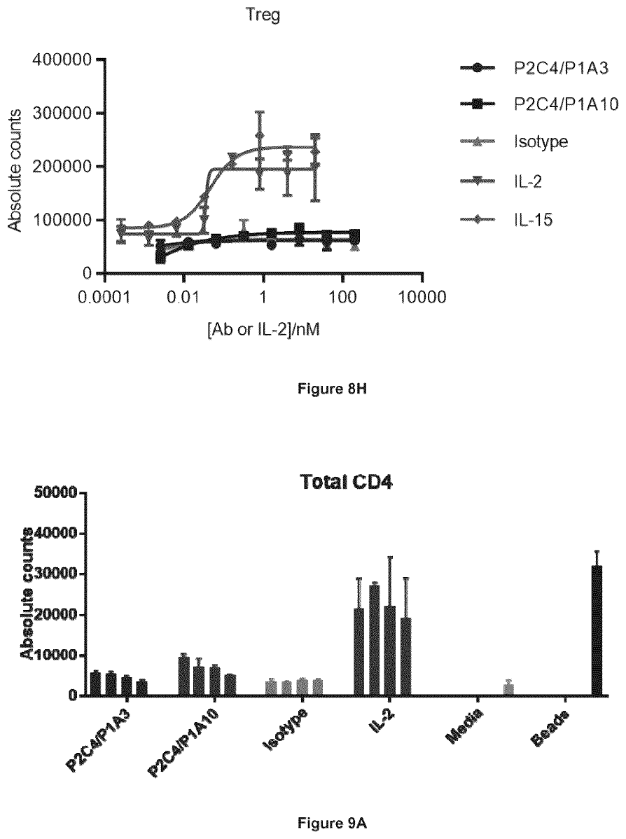

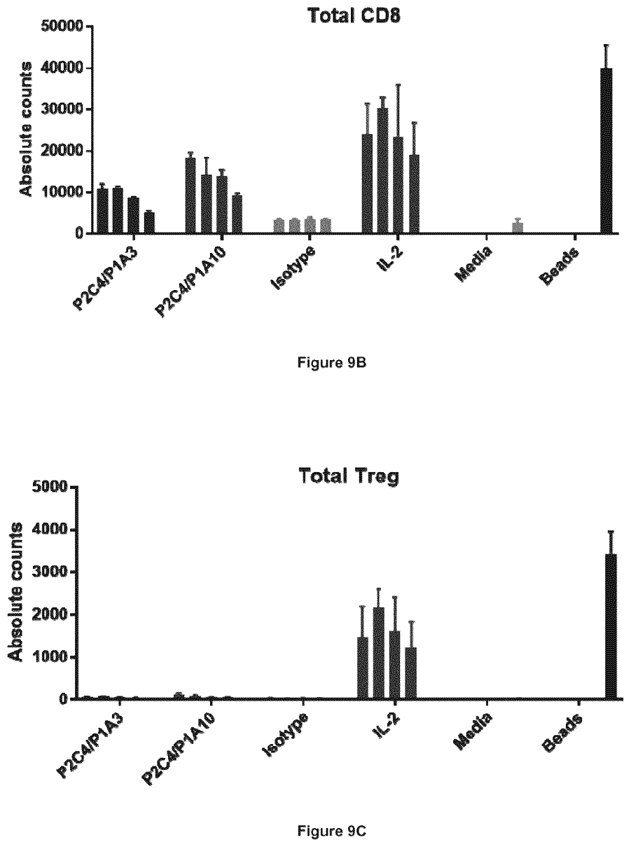

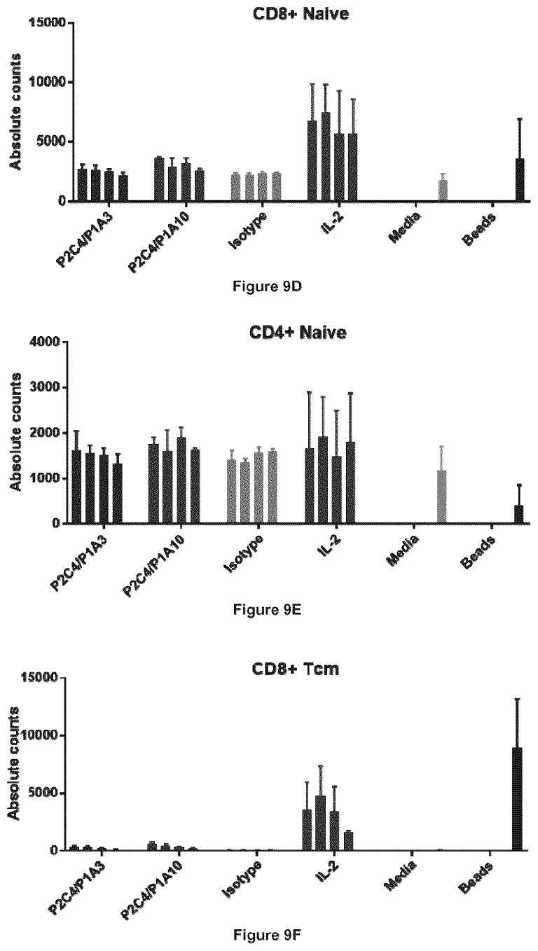

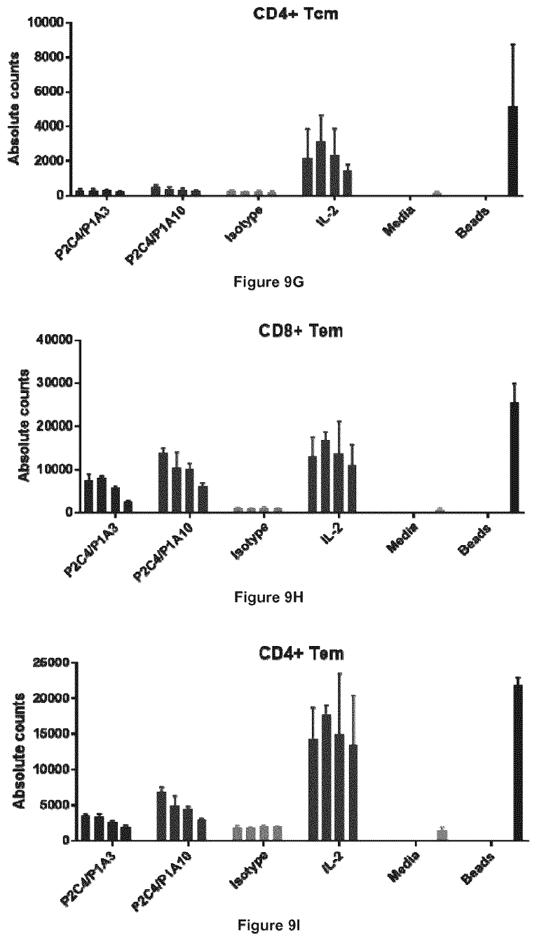

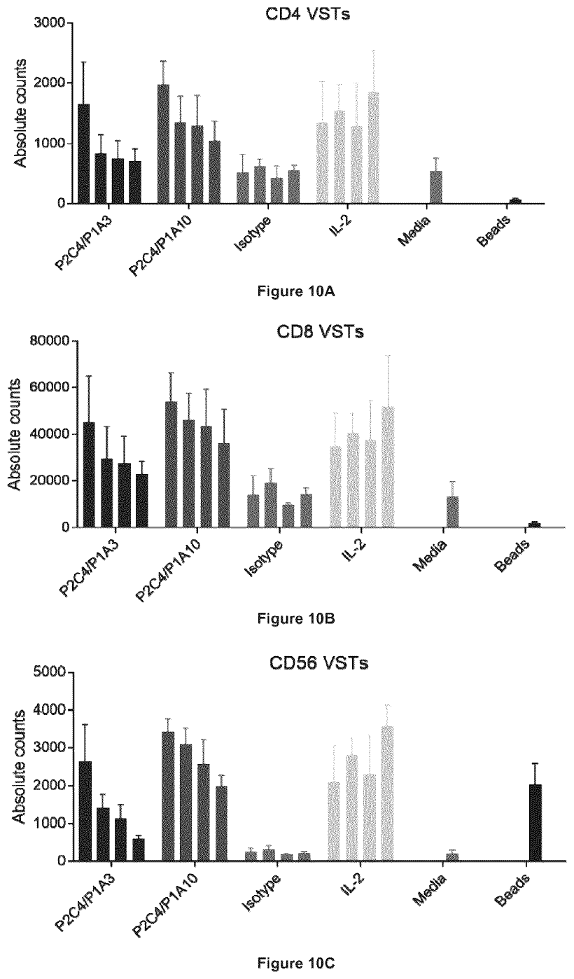

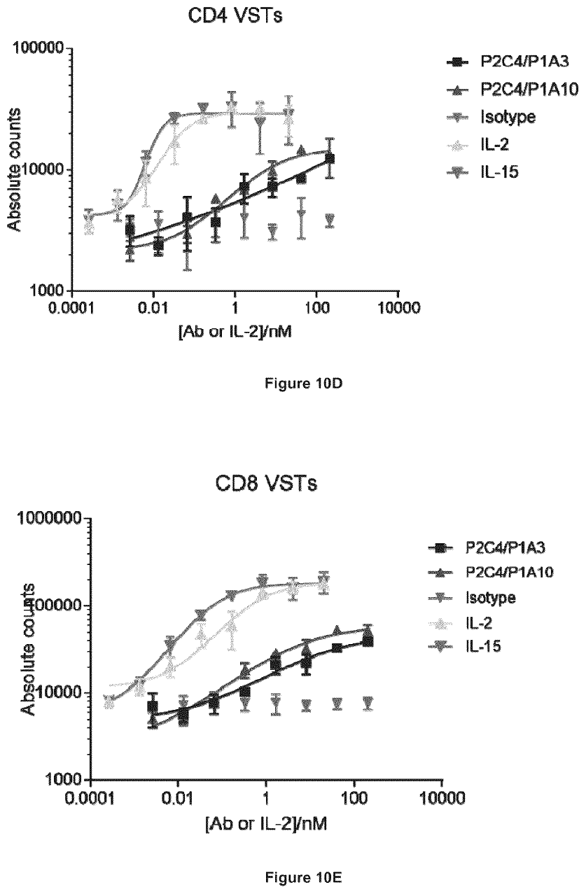

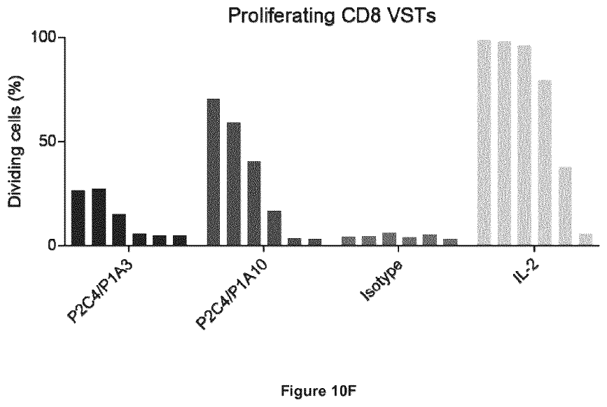

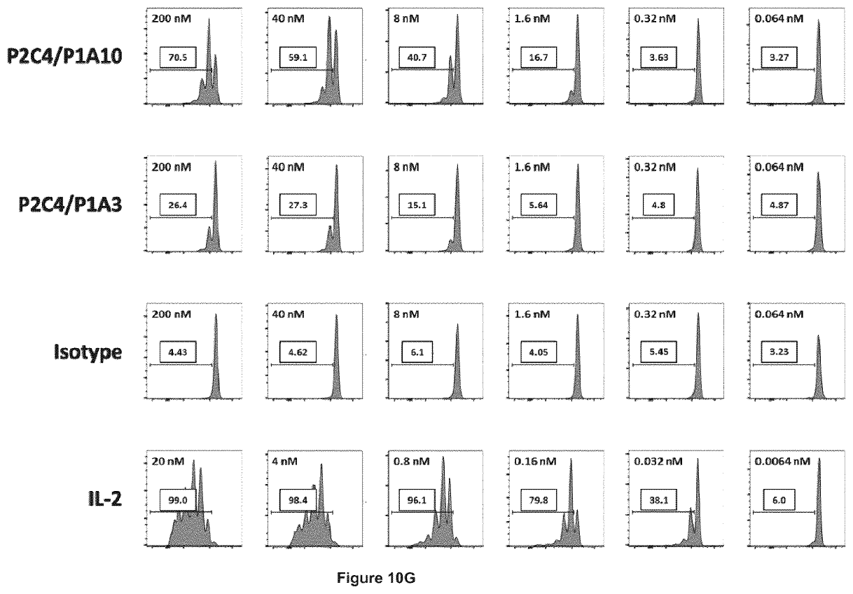

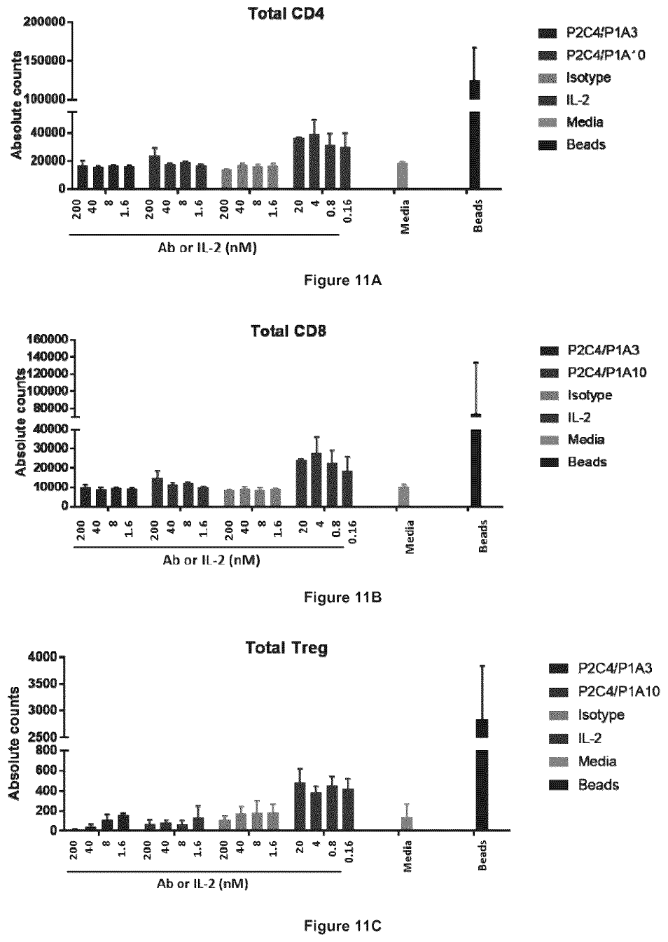

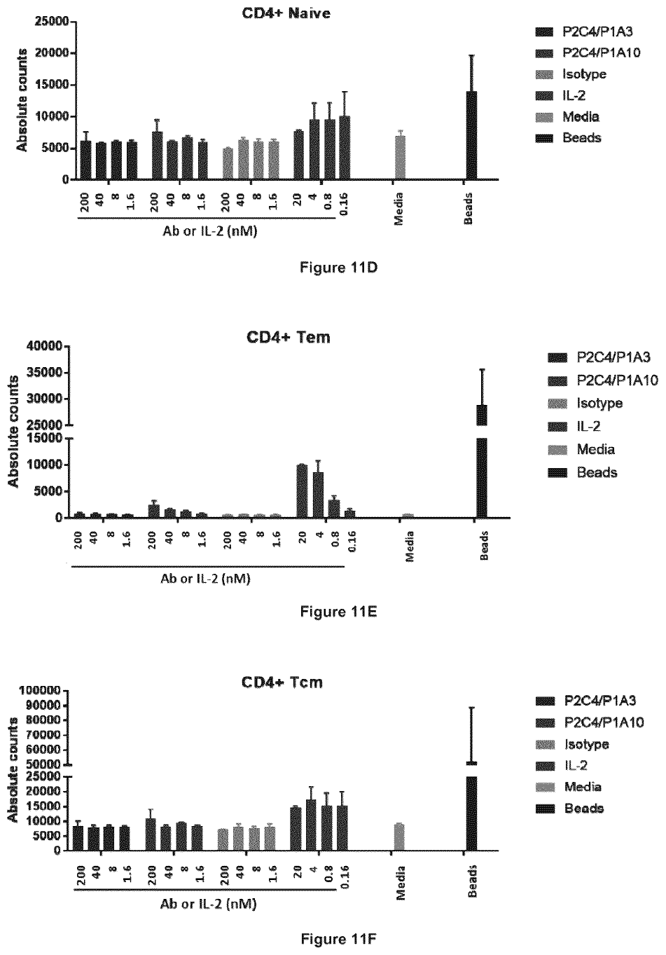

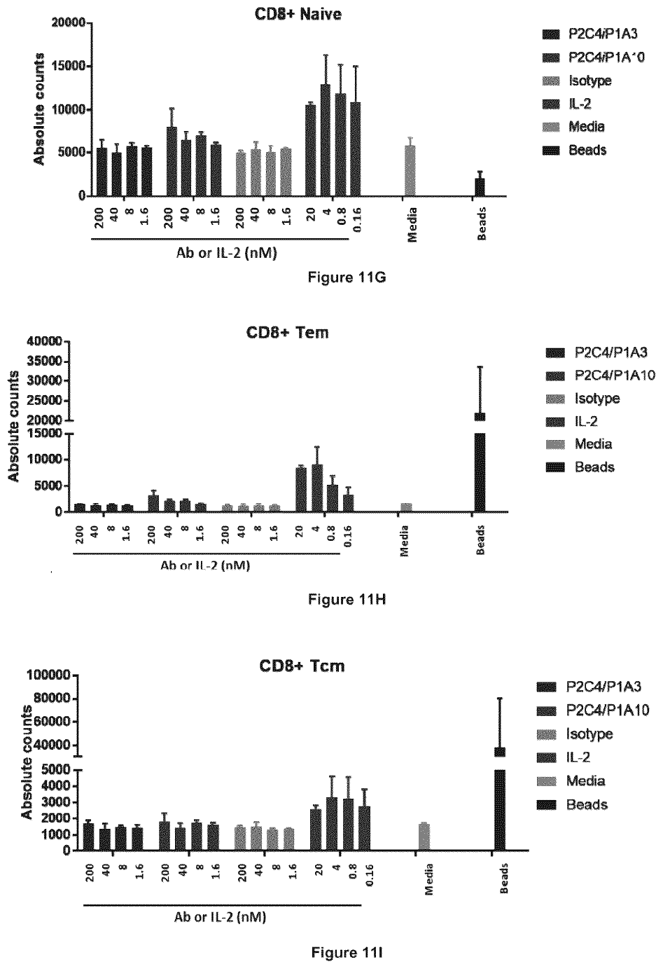

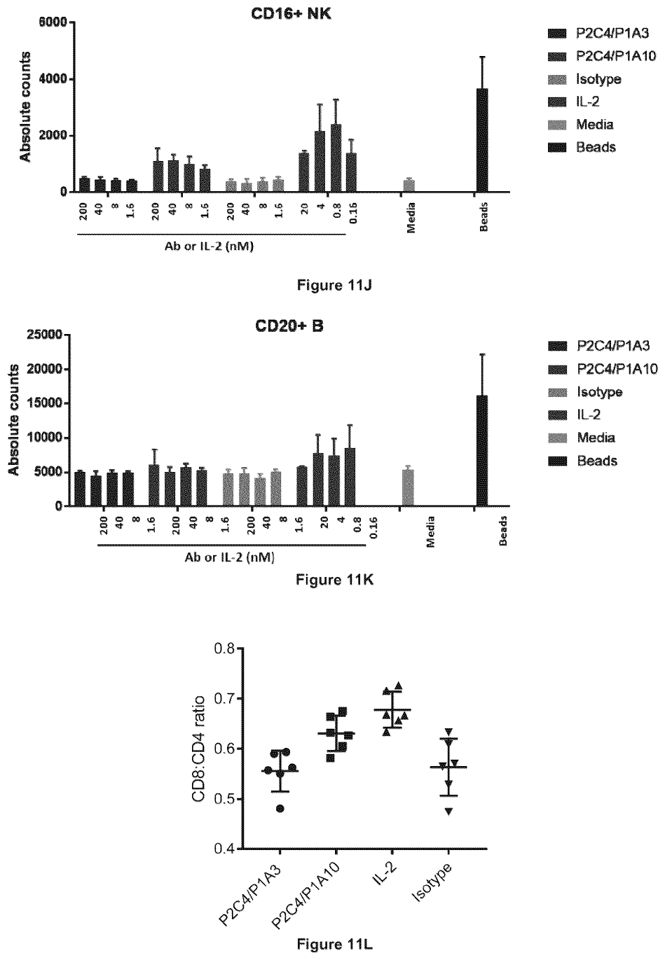

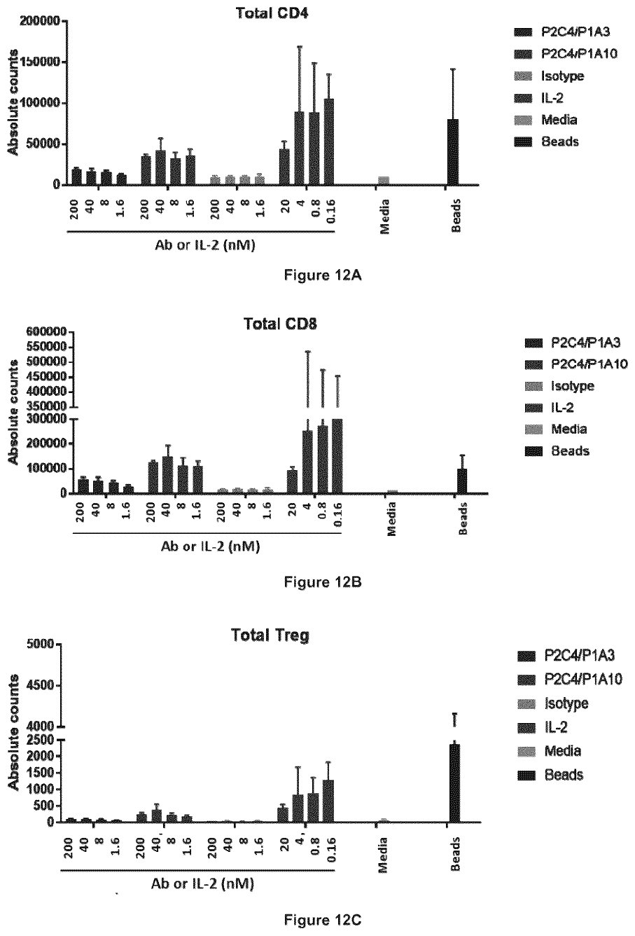

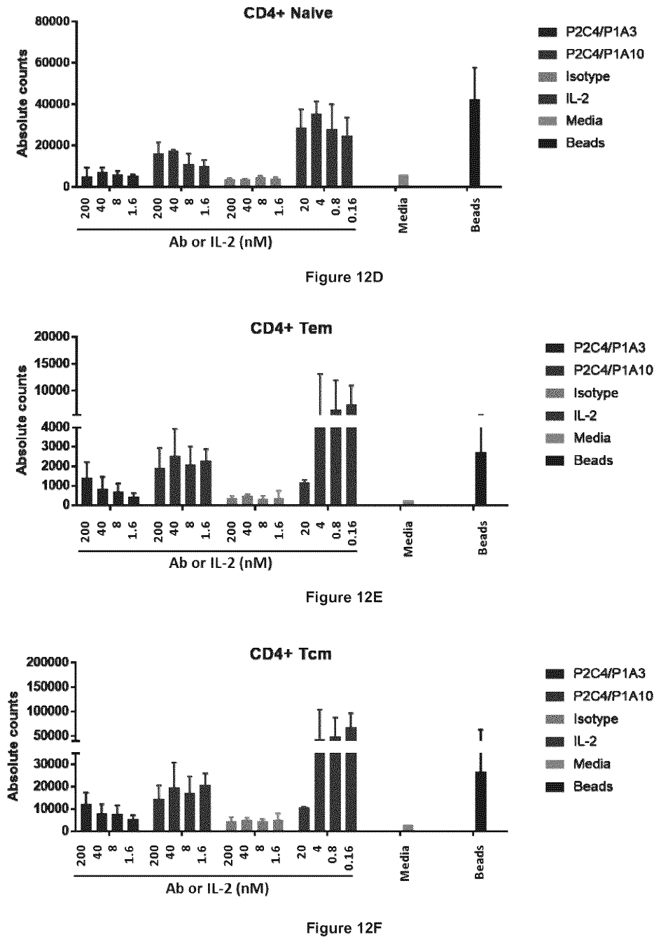

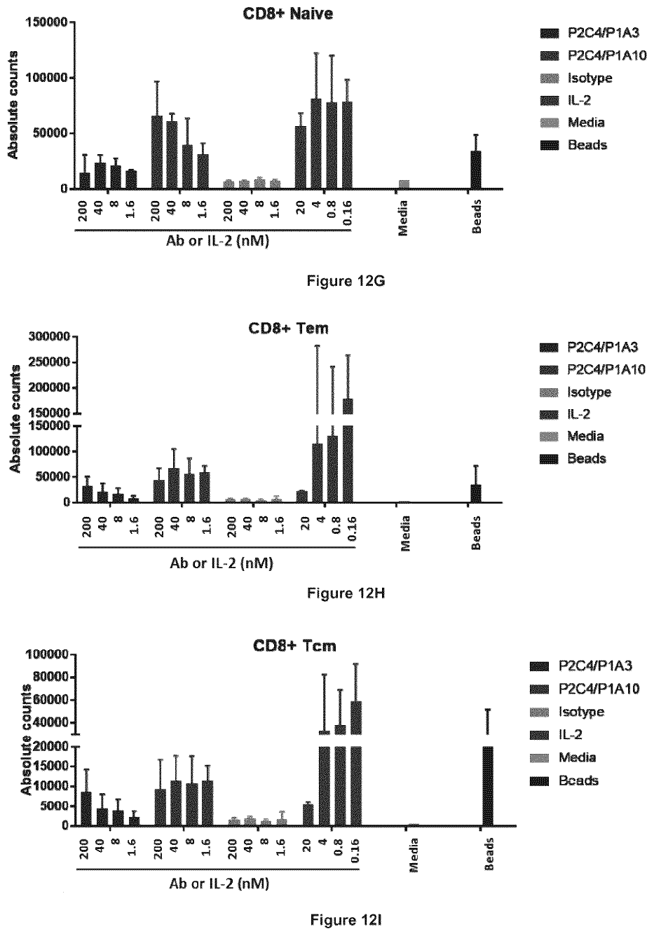

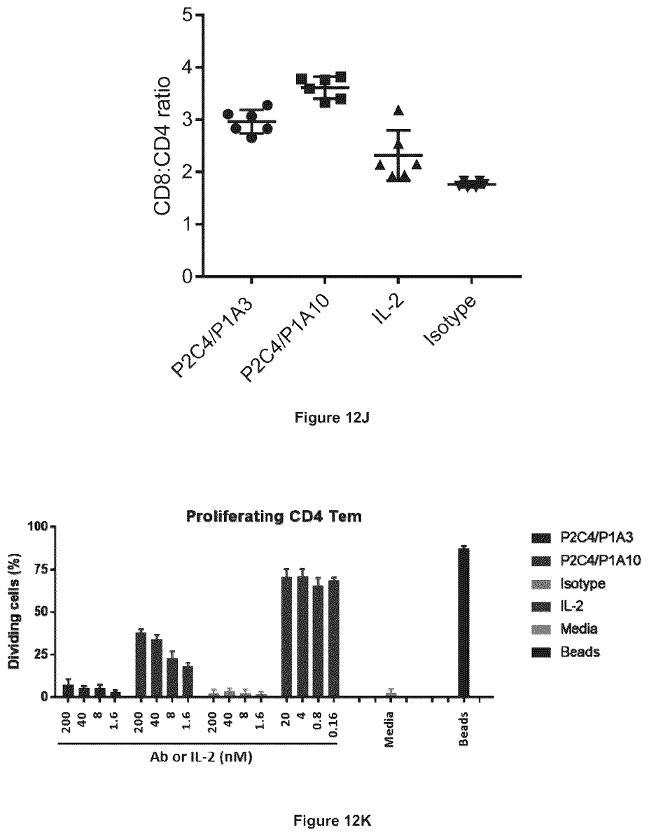

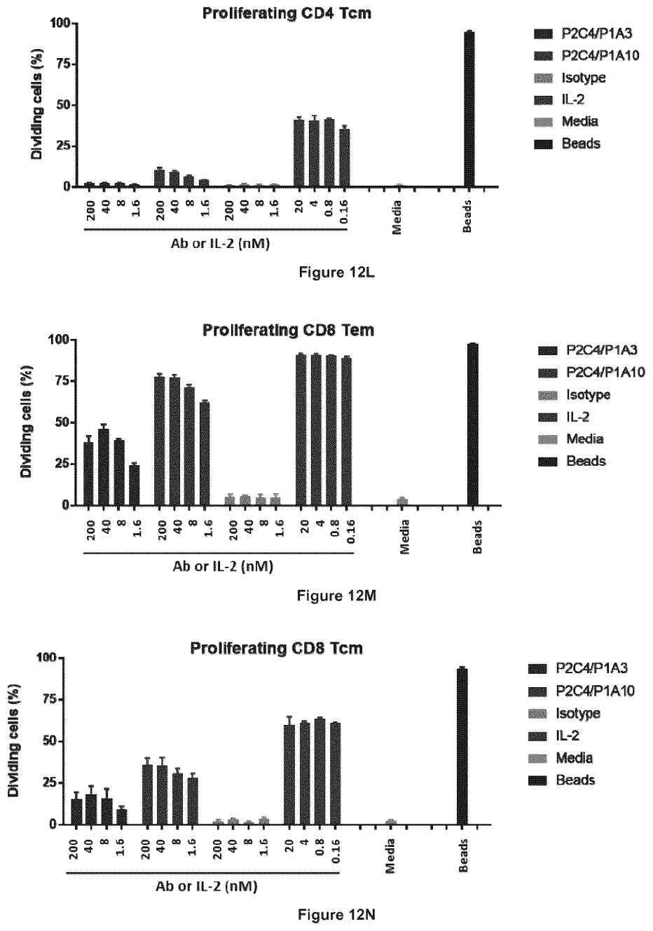

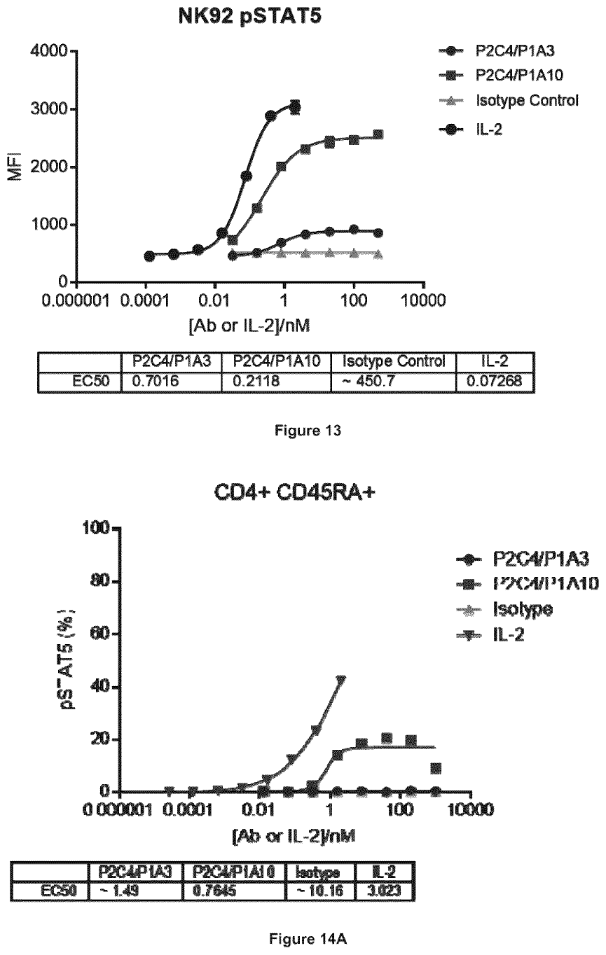

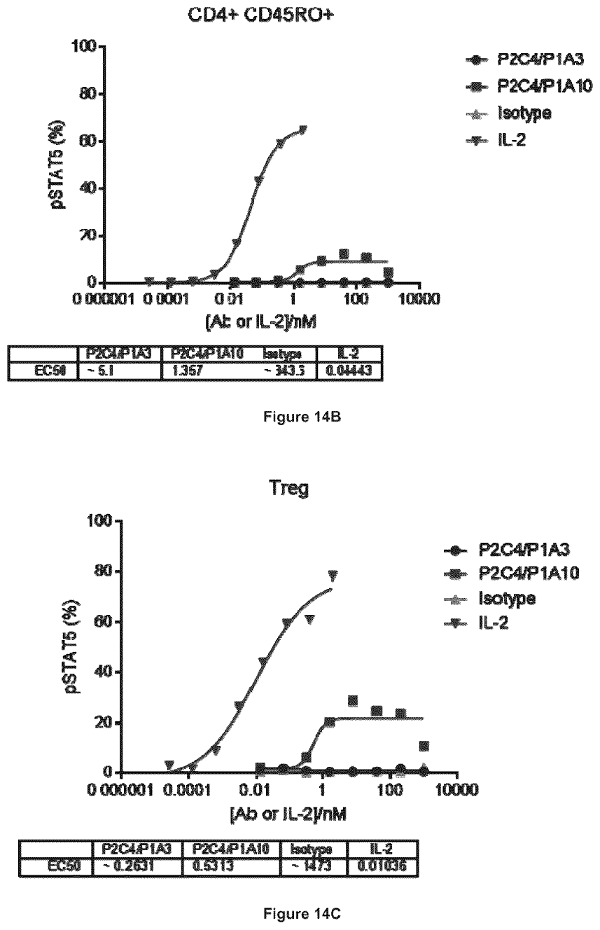

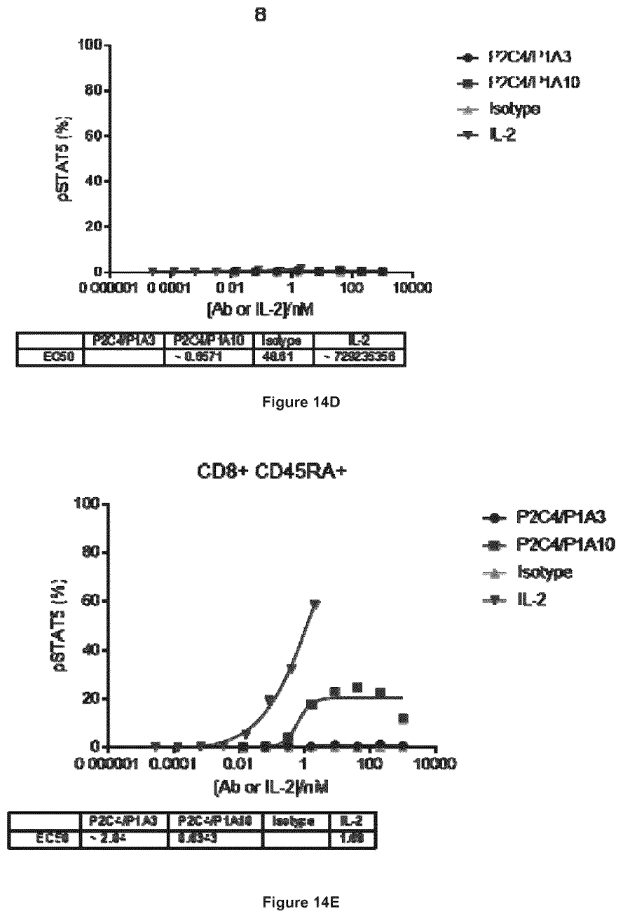

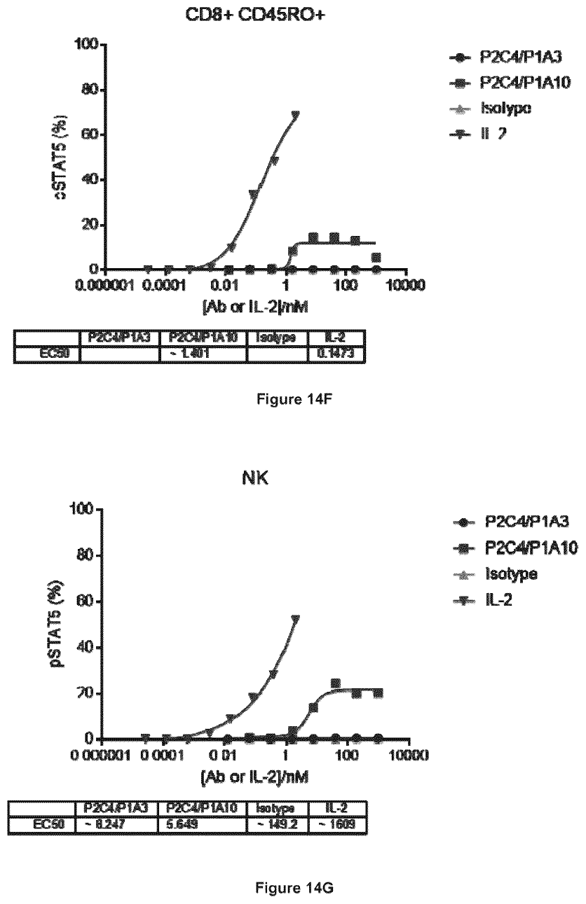

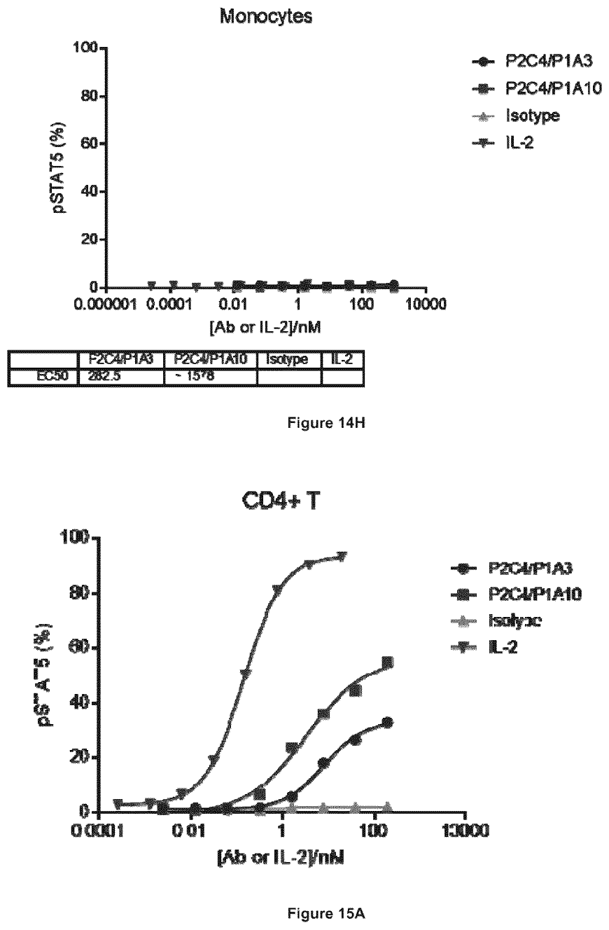

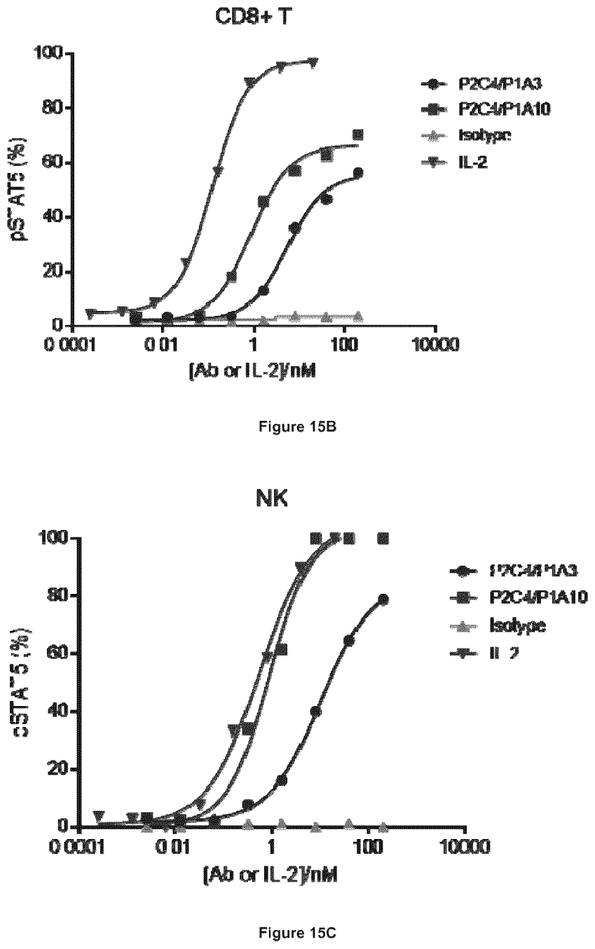

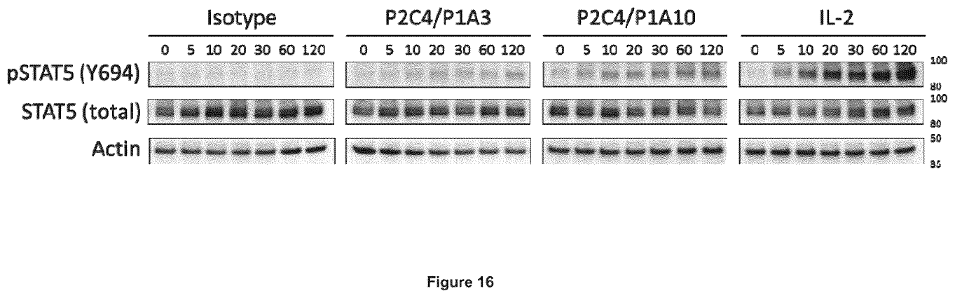

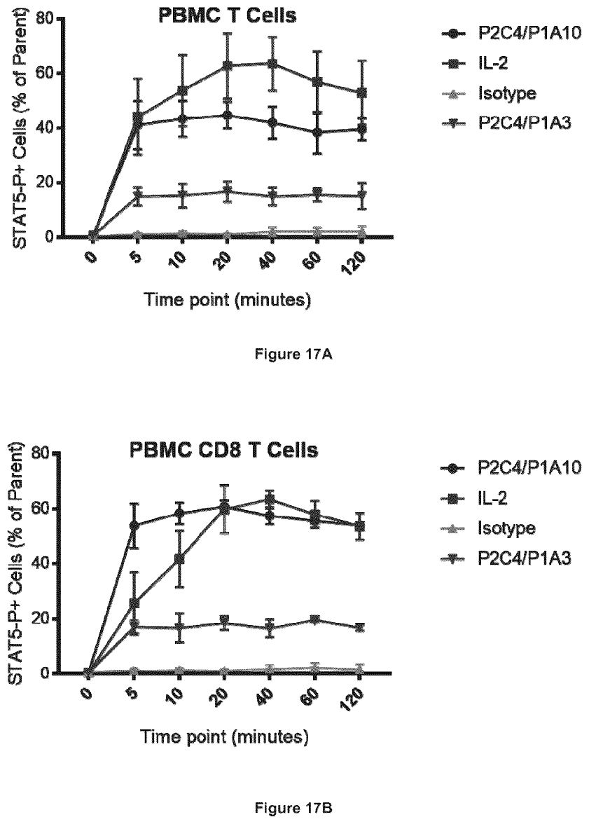

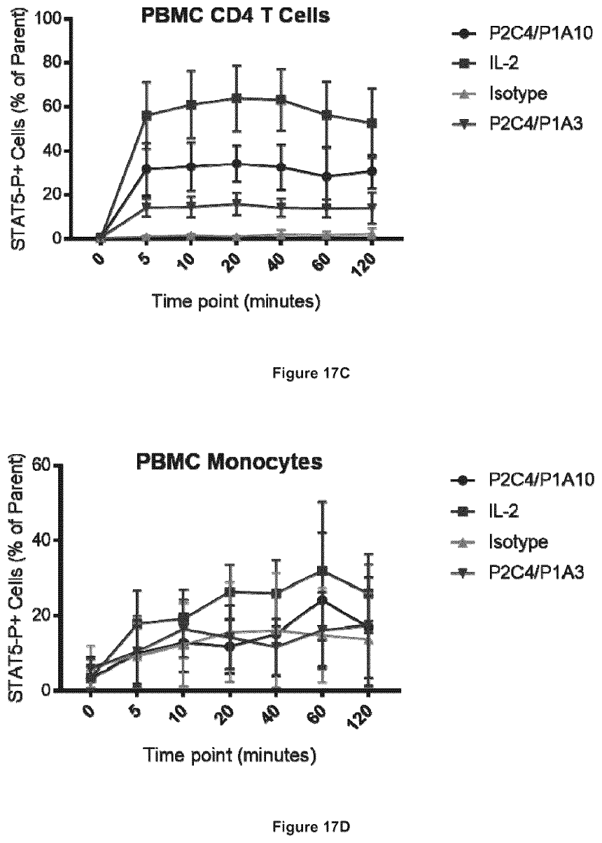

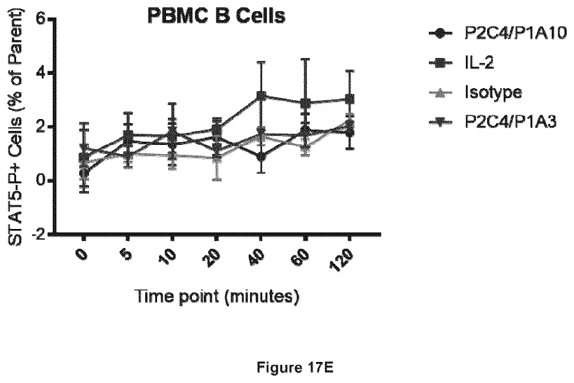

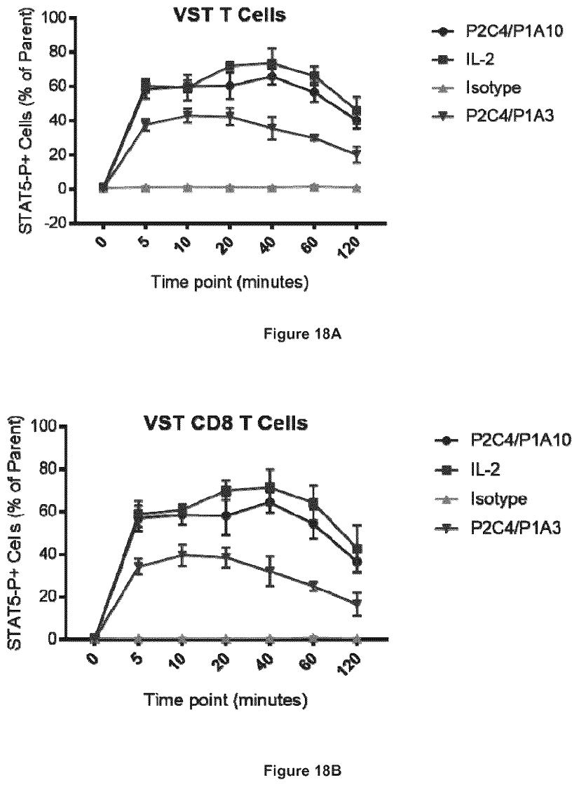

The results are shown in FIG. 7A to 7L. Treatment of pre-activated T cells with P2C4/P1A3 and P2C4/P1A10 was found to induce expansion of CD8+ T cells whilst inducing only minimal expansion of CD4+FoxP3+ regulatory T cells (Treg)—see FIGS. 7B and 7C. Absolute numbers of Trags were ˜10-fold lower following treatment with P2C4/P1A3 or P2C4/P1A10 as compared to treatment with IL-2 (FIG. 70).

With respect to the individual T cell subsets, CD8+ T effector memory subset responded the most to stimulation with P2C4/P1A3 and P2C4/P1A10 (FIG. 71). Proliferation of CD4+ T effector memory cells was also observed in P2C4/P1A10-treated cells. Based on CellTrace Violet staining, a high percentage of dividing CD8+ T effector memory cells were observed following P2C4/P1A3 and P2C4/P1A10 treatment (FIGS. 7K and 7L).

In a separate experiment, pre-activated T cells were stimulated with 8 different concentrations of P2C4/P1A3, P2C4/P1A10, isotype control antibody. IL-2 or IL-15. The ratio of CD8 to Treg cells was determined by dividing the absolute number of COB T cells with the absolute number of Tregs.

The results are shown in FIGS. 8A to 8G. Both P2C4/P1A3 and P2C4/P1A10 induced dose-dependent proliferation of pre-activated (i.e. anti-CDS/CD28 stimulated) T cells. The effect on CD8+ T cells was more pronounced than the effect on CD4+ T cells, P2C4/P1A10 was a more potent stimulator of proliferation than P2C4/P1A3. Both P2C4/P1A3 and P2C4/P1A10 did not induce significant proliferation of Tregs, with numbers similar to that of the isotype control-treated cells (see e.g. FIG. 8C). The ratio of CD8 to Treg cells indicated that both P2C4/P1A3 and P2C4/P1A10 preferentially expand CD8 T cells over Tregs, and to a greater extent than IL-2 or IL-15 (FIG. 8D). Stimulation of the CD4+ and CD8+ T effector memory T cell subsets was also dose-dependent (FIGS. 8E and 8F). A high percentage of dividing CD8+ T effector memory cells was detected following stimulation with P2CA/P1A10 or P2C4/P1A3 (FIG. 8G).

Pre-expanded human Treg cells were stimulated with P2C4/P1A3, P2C4/P1A10, isotype control antibody IL-2 or IL-15. After four days, cells were stained with CD3, CD4, CD8, Foxp3 and CD25 and assessed by flow cytometry to determine absolute counts using counting beads. Treg cells were defined as CD3+CD4+CD25+Foxp3+ cells.

The results are shown in FIG. 8H. A dose-dependent trend in the number of Treg cells was detected following stimulation with IL-2 and IL-15, but not following treatment with P2C4/P1A3 or P2C4/P1A10, indicating that neither antibody sustains nor expands Treg numbers in vitro.

3.3 Analysis of the Effect on Primary Human PBMCs

To determine whether the same stimulatory effect for P2C4/P1A3 and Gigkaine could be observed in stimulated human PBMCs. PBMCs were isolated and pre-activated with anti-CD3/CD28 beads for three days. Cells were then rested in fresh media for a day before labelling with CellTrace Violet. Cells were seeded at 400 000 per well and treated with P2C4/P1A3, P2C4/P1A10 (200 nM, 4D nM, 8 nM and 1.6 nM), IL-2 (20 nM, 4 nM, 0.8 nM, 0.16 nM) or anti-CD3/CD28 beads. Isotype antibody and untreated control conditions were included as negative controls. After four days, cells were stained with T cell markers CD3, CD4, CD8, CD45RO, CCR7, Foxp3 and CD25 to delineate T cell subsets (see Example 3.2). Counting beads were included to allow absolute cell numbers to be determined by flow cytometry.

The results are shown in FIGS. 9A to 9I. In agreement with the data obtained for treatment of pre-activated primary human T cells, P2C4/P1A3 and P2C4/P1A10 also were found to induce preferential expansion of CD8+ T cells over Tregs, and CD4+ T cell proliferation was additionally observed with 2C4/P1A10-treated cells.

3.4 Analysis of the Effect on Antigen-Specific T Cells

To determine the effects of P2C4/P1A3 and P2C4/P1A10 stimulation on antigen-specific T cells (e.g. virus-specific T cells), EBV-specific T cells (EBVSTs) were thawed and rested for a day in fresh media, and subsequently treated with P2C4/P1A3, P2C4/P1A10, IL-2 or anti-CD3/CD28 beads. Isotype antibody and untreated control conditions were included as negative controls. After four days, cells were stained with T cell markers CD3, CD4, CD8, CD45RA, CDR7, Foxp3 and CD25 to delineate T call subsets, and CD56 to enable detection of NK cells.

Counting beads were included to allow absolute cell numbers to be determined by flow cytometry.

The results are shown in FIGS. 10A to 10G. P2C4/P1A3 and P2C4/P1A10 were found to induce the expansion of both CD4+ and CD8+ virus-specific T cells (FIGS. 10A and 10B). In addition, both antibodies were able to induce the expansion of CD56+ NK cells within the virus-specific T cell population (FIG. 10C). P2C4/P1A3 and P2C4/P1A10 were found to induce proliferation of CD4+ and CD8+ virus-specific T cells in a dose-dependent manner (FIGS. 10D and 10E). A high percentage of dividing CD8+ virus-specific T cells was also detected in response to P2C4/P1A10 and P2C4/P1A3 treatment (FIGS. 10F and 10G).

3.5 Analysis of the Effect on Cynomolgus PBMCs

Frozen cynomolgus PBMCs were thawed and rested overnight in complete media before labelling with Cell Trace Violet and seeded at 200 000 cells per well. Cells were then treated with P2C4/P1A3, P2C4/P1A10, isotype antibody (200 nM, 40 nM, 8 nM, 1.6 nM) or human recombinant IL-2 (20 nM, 4 nM, 0.8 nM, 0.16 nM). Media and anti-CD3/CD28 beads were included as controls. After four days, cells were stained with T cell markers CD3, CD4, CD8, CD2B, CD95. Foxp3 and CD25 to delineate cynomolgus T cell subsets:
CD4+ Naïve T cells: CD3+CD4+CD28+CD95−
CD4+ effector memory T cells: CD3+CD4+CD28-CD95+
CD4+ central memory T cells: CD3+CD4+CD28+CD95+
CD8+ Naïve T cells: CD3+CD8+CD28+CD95−
CD8+ effector memory T cells: CD3+CD8+CD28-CD95+
CD8+ central memory T cells: CD3+CD8+CD28+CD95+

Cells were also stained with CD16 and CD2D to respectively identify NK and B cells. Counting beads were included to allow absolute cell numbers to be determined by flow cytometry.

The results are shown in FIGS. 11A to 11K. The effect of proliferation was most pronounced with IL-2 treatment. P2C4/P1A10 induced slight proliferation of CD4+, CD8+ T cells and NK cells in comparison to P2C4/P1A3 and isotype antibody control. Dose-dependent proliferation by P2C4/P1A10 was observed for CD4+ effector memory T cells. CD8+Naïve T cells. CD8+ effector memory T cells and NK cells. Treg proliferation was observed in cells treated with IL-2 but not P2C4/P1A3 or P204/P1A10.

FIG. 11L shows the ratio of the absolute number of CD8+ T cells to the absolute number of CD4+ T cells from directly stimulated cynomolgus PBMCs treated with P2C4/P1A3, P2C4/P1A10, isotype antibody or IL-2. The ratio of CD8 to CD4 T cells indicated that P2C4/P1A10 and IL-2 preferentially expand CD8 over CD4 T cells to a greater extent than P2C4/P1A3 and isotype antibody control.

3.6 Analysis of the Effect on Pre-Activated Cynomolgus PBMCs

Frozen cynomolgus PBMCs were thawed end rested overnight in complete media before pre-activation for three days with CD3/CD2/CD28 non-human primate T cell activating beads at a beads:cells ratio of 1:2. Cells were then rested in fresh media for a day before labelling with Cell Trace Violet. Cells were seeded at 180 000 per well and treated with P204/P1A3, P2C4/P1A10, isotype antibody (200 nM, 40 nM, 8 nM, 1.6 (M) or human recombinant IL-2 (20 nM, 4 nM, 0.8 nM, 0.15 nM). Media and anti-CD3/CD28 beads were included as controls. After four days, cells were stained with T cell markers CD3, CD4, CD8, CD28, CD95, Foxp3 and CD25 to delineate T cell subsets, as above.

Counting beads were included to allow absolute cell numbers to be determined by flow cytometry.

The results are shown in FIGS. 12A to 12I. P2C4/P1A3 and P2C4/P1A10 induced the proliferation of both CD4+ and CD8+ T cells but not Treg. P2C4/P1A10 induced the proliferation of all CD4+ and CD8+ T cell subsets. Dose dependent proliferation was observed in both CD4+ and CD8+ effector memory and central memory T cells under P2C4/P1A3 treatment.

FIG. 12J shows the ratio of the absolute number of CD8+ T calls to the absolute number of CD4+ T cells from pre-activated cynomolgus PBMCs treated with P2C4/P1A3, P2C4/P1A10, isotype antibody or IL-2. The ratio of CD8 to CD4 T cells indicate that P2C4/P1A3 and P2C4/P1A10 preferentially expand CD8 over CD4 T cells to a greater extent than IL-2 and isotype antibody control.

FIGS. 12K to 12N show analysis of proliferation of CD8+ and CD4+ T cells. A high percentage of dividing CD8+ T effector memory and CD8+ T central memory cells were detected following stimulation with P2C4/P1A10 or P2C4/P1A3. In addition, P2C4/P1A10 also induced a high percentage of dividing CD4+ T effector memory and CD4+ T central memory cells.

3.7 Analysis of the Effect on Pre-Activated Human NK Cells

To determine the effects of IL-2Rβ- and γc-binding bispecific antibodies on human NK calls, primary NK cells were isolated from human PBMCs and pre-activated for three days with irradiated K562-4-1BBL-CD64-CD86 cell line. Cells were labelled with CellTrace™ Violet for tracking of cell proliferation, then treated with P2C4/P1A3, P2C4/P1A10, IL-2 and IL-15. Isotype antibody and no treatment wells were included as negative controls. After five days, cells were stained for CD56 and CD16 NK cell markers. Counting beads were also added to determine the absolute cell numbers, and samples were analysed by now cytometry.

The results are shown in FIGS. 27A to 27D. P2C4/P1A3 and P2C4/P1A10 were both found to induce proliferation of activated NK cells. Both the CD56+CD16+ and CD56+CD16− NK subsets were found to proliferate in response to P2C4/P1A3 and P2C4/P1A10 treatment in a dose-dependent manner, as shown by the dose-dependent increase in absolute counts of both types of NK cells (27A, 27B) and the percentages of the corresponding proliferating NK cell subsets Illustrated by CellTrace™ Violet (CTV)-diluted cells (27C, 27D).

3.8 Analysis of the Effect on CAR-T Cells

To determine the effects of IL-2Rβ- and γc-binding bispecific antibodies on CAR-T calls, primary T cells were isolated from human PBMCs and then transduced with the CAR construct so that the CAR is expressed. Alter 24 days of cell expansion, cells were labelled with CellTrace™ M Violet for tracking of cell proliferation, then treated with P2CA/P1A3, P204/P1A10, isotype antibody or IL-2. After five days, cells were stained for CAR expression and T cell markers to identify the T cell subsets Counting beads were also added to determine the absolute cell numbers, and samples were analysed by flow cytometry.

The results are shown in FIGS. 28A to 28D. P2C4/P1A3 and P2C4/P1A10 both induce proliferation of CAR-T cells. The antibodies were found to induce expansion of the CD4+ and CD8+ CAR-T cells in a dose-dependent manner, as shown by the dose-dependent increase in absolute counts of CD4+(28A) and CD8+(28B) CAR-T call subsets A greater percentage of CD8+ CAR-T cells were found to be proliferating (28D) compared to CD4+ CAR-T cells (28C), determined by CellTrace™ Violet (CTV), suggesting that CD8+ CAR-T cells are more responsive to P2C4/P1A3 and P2C4/P1A10 treatment then CD4+ CAR-T cells.

### Example 4: Analysis of Induction of Intracellular Signalling by IL-2RB- and γc-Binding Bispecific Antibodies

4.1 Analysis of Induction of STAT5 Phosphorylation in NK Cells

NK92 cells were washed and rested in IL-2-free media for 1 h prior to stimulation with various concentrations of P2C4/P1A3, P2CA/P1A10, Isotype control antibody or IL-2 for 30 min. Cells were subsequently fixed, permeabilised and stained for phosphorylated STAT5 using a fluorescently-labelled antibody, and the samples were then analysed by flow cytometry.

The results are shown in FIG. 13. Both P2C4/P1A3 and P2C4/P1A10 were found to stimulate phosphorylation of STAT5 in NK92 cells in a dose-dependent manner. Activation by P2C4/P1A10 achieved a higher level of STAT5 phosphorylation as compared to activation by P2C4/P1A3.

4.2 Analysis of Induction of STAT5 Phosphorylation in Primary Human Immune Cell Subsets

PBMCs were thawed and rested overnight prior to being seeded at 400.000 cells per well in serum-free media. Cells were rested for two hours and subsequently stimulated with 8 different concentrations of P2C4/P1A3, P2C4/P1A10, isotype control antibody or IL-2. After 30 minutes, cells were analysed by flow cytometry for phosphorylation of STAT5 as well as for immune cell markers CD3, CD4, CD8, CD45RA, CD45RO, Foxp3, CD25, CD56, CD19 and CD14 to delineate T subsets, B, NK cells and monocytes.

The results are shown in FIGS. 14A to 14H. P2C4/P1A10 induced phosphorylation of STAT5 in several T cell subsets, as well as in NK cells, in a dose-dependent manner. P2C4/P1A3 induced minimal phosphorylation of STAT5. Minimal phosphorylation of STAT5 was also observed in B cells and monocytes.

4.3 Analysis of Induction of STAT5 Phosphorylation in Pre-Activated Primary Human Immune Cell Subsets

PBMCs were thawed and rested overnight before pre-activation with anti-CD3/CD28 beads for three days. Cells were then rested in fresh media for a day before seeding at 200 000 par well in serum-free media. Cells were rested for two hours and subsequently stimulated with 8 different concentrations of P2C4/P1A3, P2C4/P1A10, isotype control antibody or IL-2. After 30 minutes, cells were analysed by flow cytometry for phosphorylation of STAT5 as well as for immune cell markers CD3, CD4, CD8, and CD56 to delineate T subsets and NK cells.

The results are shown in FIGS. 15A to 15C. Both P2C4/P1A10 and P2C4/P1A3 induced the phosphorylation of STAT5 within pre-activated CD4+, CD8+ T cell subsets and NK cells in a dose-dependent manner, and to a greater extent than within non-activated cells.

4.4 Analysis of the Kinetics of Induction of STAT5 Phosphorylation in NK Cells

NK92 calls were rested in serum-free media and treated with P2C4/P1A3, P2C4/P1A10. Isotype control antibody at 100 nM or IL-2 at 20 nM for 5, 10, 20, 30, 60 and 120 min. Cells were harvested at the indicated time points for assessment of STAT5 phosphorylation (Y694) via western blotting. Total STAT5 and actin were included as controls.

P2C4/P1A3 and P2C4/P1A10 were able to induce pSTAT5 in a time-dependent manner in comparison to Isotype antibody treatment (FIG. 16).

4.5 Analysis of the Kinetics of Induction of STAT5 Phosphorylation in Primary Human Immune Cell Subsets

Freshly isolated human PBMCs were stimulated with 50 nM P2C4/P1A3, P2C4/P1A10, Isotype control antibody or 2 nM IL-2 in a reverse lime course of 0, 5, 10, 20, 40, 60 and 120 min. Cells were then fixed, permeabilised and stained with CD3, CD4, CD8, CD14, CD19 and pSTAT5 (Y694) for the identification of immune cell subsets. The data are presented as mean percentage of pSTAT5-positive cells of the PBMC subsets from 3 donors.

The results are shown in FIGS. 17A to 17E. Both P2C4/P1A3 and P2G4/P1A1D induced STAT5 phosphorylation in T cells. Maximal stimulation was achieved by both antibodies at the 5 min time point P2C4/P1A10 also stimulated a higher percentage of pSTAT5-positive cells than P204/P1A3 over the time course of 2 h. Stimulation of PBMCs with P2C4/P1A3 and P204/P1A10 did not result in a significantly greater percentage of pSTAT5-positive monocytes and B cells as compared to the isotype control antibody.

4.6 Analysis of the Kinetics of Induction of STAT5 Phosphorylation in Antigen-Specific T Cells

EBV-specific T cells were thawed and rested in fresh media, and subsequently stimulated with 50 nM P2C4/P1A3, P2C4/P1A10, isotype control antibody or 2 nM IL-2 in a reverse time course of 0, 5, 10, 20, 40, 60 and 120 min. Cells were then fixed, permeabilised and stained with CD3, CD4, CD8 and pSTAT5 (Y694) for the identification of EBV-specific T cell subsets. The data are presented as mean percentage of pSTAT5-positive cells of the virus-specific T cell subsets from 3 donors.

The results are shown in FIGS. 18A to 18C. Similar to the data obtained using human PBMCs (Example 4.5), both P2C4/P1A3 and P2C4/P1A10 stimulated STAT5 phosphorylation in EBV-specific T cells, and P2C4/P1A10 induced higher percentage of STAT5-positive cells than P2C4/P1A3 over the time course of 2 h.

4.7 Effects on Other Cytokine Receptors

To assess whether P2C4/P1A3 or P2C4/P1A10 binding of IL-2Ry prevents IL-4 signalling through the IL-4 receptor, THP-1 cells were treated with P2C4/P1A3, P2C4/P1A10. Isotype Control antibody (100 nM) or IL-2 (20 nM), with or without IL-4 (200 ng/ml) for 30 minutes. Cell lysates were assessed by western blotting to determine phosphorylation of STATE (Y641). Total STAT6 and actin were included as controls.

IL-4 induced pSTAT6 to a similar extent between different conditions, even in the presence of P2C4/P1A3/P2C4/P1A10 (FIG. 19). This suggests that despite binding to IL-2Rγ, P2C4/P1A3 and P2C4/P1A10 do not affect IL-4-mediated signalling.

### Example 5: Analysis of Toxicity—Induction of Proliferation of Non-Activated Immune Cells

5.1 Analysis of Stimulation of Proliferation by Non-Activated PBMCs

To measure the effects of P2C4/P1A3 and P2C4/P1A10 on non-activated, freshly-obtained PBMCs. PBMCs were isolated and directly treated with P2C4/P1A3, P2C4/P1A10 (200 nM, 40 nM, 8 nM and 1.6 nM), IL-2 (20 nM, 4 nM, 0.8 nM and 0.16 nM) or anti-CD3/CD28 beads as positive control. Isotype antibody and untreated control conditions were included as negative controls. After four days, cells were stained with T cell markers CD3, CD4, CD8, CD45RO, CCR7, Foxp3 and CD25 to for the delineation of T cell subsets, and with CD19 and CD56 for the identification of 8 cells and NK cells, respectively. Counting beads were included to enable absolute cell numbers to be determined by flow cytometry.

The results of the analysis are shown in FIGS. 20A to 20K. P2C4/P1A3 and P2C4/P1A10 did not induce significant proliferation of non-activated PBMCs as compared to isotype control antibody. This was observed for all T cell subsets including CD4, CD8, Treg, naïve, T central memory (Tcm) and T effector memory cells (Tem), as well as NK cells. This contrasts with IL-2, which stimulated expansion of T and NK calls even at lower doses. Minimal proliferation was also observed for B cells in response to treatment with IL-2.

T cell activation requires three signals (1) TCR-(CD3)/MHC interaction, (2) co-stimulation i.e. CD28 and (3) cytokine signalling i.e. IL-2, As P2C4/P1A3 and P2C4/P1A10 do not induce proliferation of T cells under direct stimulation, this indicated that signals (1) and (2) are required before T cells become responsive to the antibodies, in agreement with the results obtained with preactivated calls.

These data suggest that P2C4/P1A3 and P2C4/P1A10 preferentially expand activated T cells, and may be associated with reduced toxicity as compared to treatment with IL-2 (which expands both activated and non-activated cells).

5.2 Analysis of Stimulation of Proliferation by Non-Activated T Cells

Human T cells were isolated and directly treated with P2C4/P1A3, P2C4/P1A10. IL-2 or anti-CD3/CD28 beads. Isotype antibody and untreated control conditions were included as negative controls. After four days, cells were stained with T cell markers CD3, CD4, CD5, CD45RA, CCR7, Foxp3 and CD25 to delineate T cell subsets. Counting beads were included to enable absolute cell numbers to be determined by flow cytometry.

The results are shown in FIGS. 21A to 21C. Similar to the observation with direct stimulation of human PBMCs, P2C4/P1A3 and P2C4/P1A10 did not induce proliferation of T cells without pre-activation, indicating that antigen recognition/CD3 activation and co-stimulation signals are required before T cells become responsive to these antibodies. This contrasts with IL-2, which indiscriminately expanded T cells even at low doses.

### Example 6: Analysis of Pharmacokinetics in non-Human Primates

A simple pharmacokinetics (PK) study was performed to measure the clearance of P2C4/P1A3 in non-human primates.

3 cynomolgus macaques were injected with a single dose of 1 mg/kg, 5 mg/kg and 10 mg/kg P2C4/P1A3 and blood collection was done at pre-dose, 1 h, 24 h, 72 h and 120 h post-antibody injection time-points. Plasma is obtained from the collected blood and a sandwich ELISA were performed to measure the levels of P2C4/P1A3.

Sandwich ELISA was performed using coated anti-human CH2 antibody, and detection of P2C4/P1A3 was by using anti-human Fc-HRP. An ELISA standard curve was derived using purified P2C4/P1A3 for calculation of the absolute concentration of antibody in blood.

The results are shown in FIG. 22. Maximum blood antibody levels were detected at 1 h post antibody dosing, and remained in the system up to 120 h.

IL-2 is known to have a much shorter serum half-life—see e.g. Skrombolas and Frelinger, Expert Rev Clin Immunol. (2014)10(2): 207-217, which reports that a study of the serum half-life of IL-2 introduced intravenously found a bi-phasic event with phase I (biodistribution throughout the body) resulting in t_{1/2} of approximately 7 min and phase II (extravasation from plasma into tissue) at approximately 60 min.

### Example 7: Analysis of IL-2RS and Yc Expression on Human PBMCs and Antigen-Specific T Cells

Human PBMCs were thawed and rested overnight in cell culture media. The cells were then activated using anti-CD3/CD28 beads.

After three days, cells were rested in media for a day before staining with commercially available anti-IL-2Rβ or γc antibodies plus antibodies for markers of human immune subsets. Cells were then analysed by flow cytometry to determine the expression of IL-2Rβ and γc before (−) and after (+) pre-activation. Normalized Median Fluorescence Intensity (nMFI) was calculated by subtracting MFI values for the “fluorescence-minus one” (FMO) control.

The results are shown in FIGS. 23A and 23B. Activation of human PBMCs with anti-CD3/CD28 was shown to upregulate surface expression of both IL-2Rβ and ye across three different donor samples, particularly on T cell subsets.

In a separate experiment, EBV-specific immune cells were thawed and rested in fresh media overnight prior to being stained with commercially available anti-IL-2Rβ or γc antibodies plus antibodies for markers of human T cell subsets and NK cells. Cells were then analysed by flow cytometry to determine the expression of IL-2Rβ and γc. Normalized Median Fluorescence Intensity (nMFI) was calculated by subtracting MFI values for the “fluorescence-minus one” (FMO) control.

The results are shown in FIGS. 24A and 24B. Expression of IL-2Rβ and γc was detected on different immune cell subsets within the EBV-specific T cells derived from three different donors.

### Example B: Production of Anti-IL-2Rβ/γc Antibody P2C4IP1A10 in Duobody Format

P2C4/P1A10 was made in the Duobody format. Briefly, monospecific anti-IL-2Rβ P2C4 IgG1-K409R and anti-γc P1A10 IgG1-F405L antibodies were produced and purified, mixed, then subjected to reduction with 75 mM 2-MEA at pH 8.5, 31° C. for 5 h, 2-MEA was removed by dialysis, and the antibodies were left to re-oxidise at 4° C. The fully formed bispecific Duobody were purified by anion exchange chromatography.

### Example 9: Analysis of the Effect of Anti-IL-2Rβ/γc Antibodies on Anti-Cancer Immune Responses

Example 8.4 of WO 2017/021540 A1 reports the ability of CD8+ T cells expanded by treatment with bispecific agonist anti-IL-2Rβ and -γc antibodies to kill cancer cells. Specifically, T cells expanded from PBMCs obtained from EBV seropositive donors by culture in presence of P2C4:P1A3 are shown to kill LCLs.

Example 12 and FIGS. 41 and 42 of WO 2017/021540 A1 demonstrate the ability of bispecific agonist anti-HL-2Rβ and -γc antibodies to stimulate proliferation of T cells and NK cells in vivo in cynomolgus macaques.

In the present Example, bispecific agonist anti-IL-2Rβ and -γc antibodies are shown to promote an anti-cancer immune response in vivo.

Tumours are established by subcutaneous injection of mice with LOLs. Specifically. EBV-transformed lymphoblastoid B-cell line (LCLs) was mixed with Matrigel and injected subcutaneously to the right flank of NSG mice.

Mice were subsequently administered with autologous EBV-specific CTLs (VSTs), with or without P2C4/P1A3, P2C4/P1A10, isotype control antibody, or IL-2, at 19 days post-tumour inoculation, IL-2 treatments were given at 40 000 U/kg, intra-peritoneally (i.p.) consecutively for 5 days for a total of 5 doses. Antibody treatments were given at 5 mg/kg, i.p. every 14 days, for a total of 2 doses. The administration schedule is shown in FIG. 25.

Mouse blood was collected at 8 days post-VST treatment and flow cytometric analysis showed elevated numbers of total human CD3, CD4 and CD8 T cells in mice treated with P2C4/P1A3 and P2CA/P1A10 as compared to mica treated with isotype control antibody or IL-2. The results are shown in FIGS. 26A to 26C.

At the end of the experiment, mice were euthanised at 22 days post-VST treatment and blood, spleen, liver, tumour-draining lymph node and flank tumour were harvested for flow cytometric analysis.

The results are shown in FIGS. 26D to 26H. Similar to results at 8 days post-VST treatment, mice treated with P2C4/P1A3 and P2C4/P1A10 had elevated numbers of total human CD3, CD4 and COB T cells in blood and organs. CD3 T cells from mice treated with P2C4/P1A3 and P2C4/P1A10 also had lower expression of PD-1 as compared to cells from mice treated with IL-2 and isotype control antibody.

Total organ tumour load in mice was calculated from the total numbers of GD 19+ cells in spleen, liver, flank tumour and tumour-draining lymph node (FIG. 261). Mice treated with P2CA/P1A3 and P2C4/P1A10 had lower total organ tumour burden as compared to mice treated with isotype control antibody, IL-2 or no VSTs.

### Example 10: Analysis of the Effect of anti-IL-2Rβ/γc Antibodies on Anti-Cancer Immune Responses in the Presence of Tregs

In the present Example, bispecific agonist anti-IL-2Rβ and -γc antibodies are shown to promote an anti-cancer immune response in vivo, without accompanying increases in immunosuppressive regulatory T cells (Tregs) in a mouse model of EBV-BLCL metastatic lymphoma.

Tumours are established by subcutaneous injection of mice with LCLs. Specifically. EBV-transformed B lymphoblastoid cell lines (LCLs) was mixed with Matrigel and injected subcutaneously to the right flank of NSG mice.

Mice were subsequently administered with autologous EBV-specific CTLs (VSTs) and Tregs, with or without P2C4/P1A3, P2C4/P1A10, isotype control antibody, or IL-2, at 20 days post-tumour inoculation.

IL-2 treatments were given at 40 000 U/kg, intra-peritoneally (i.p.) consecutively for 5 days for a total of 5 doses Antibody treatments were given at 5 mg/kg, i.p. every 14 days, for a total of 2 doses. The administration schedule is shown in FIG. 29.

Analysis of peripheral blood collected from mice at 7, 14 and 21 days post-treatment showed that slightly higher numbers of circulating VSTs were detected in mics which received bispecific anti-IL-2Rβ and -γc antibodies compared to isotype (Iso) or IL-2, as shown in FIG. 30A.

At the end of the experiment, mice were euthanised at 22 days post-treatment and spleen, liver, tumour-draining lymph node and injection site were harvested for flow cytometric analysis. The results are shown in FIGS. 30B to 30E. Mice Treated with P2C4/P1A3 and P2C4/P1A10 were found to have elevated numbers of total human CD3, CD4 and CD8 T cells in spleen (30B), liver (30C), tumour-draining lymph node(s) (30D) and injection site (30E). **, p<0.01: *, p<0.05.

The ratio of CD8 cells to Tregs has been shown to be predictive of a favourable outcome in multiple tumour types (de Leeuw R J et al. Clin Cancer Res 2012, 18:3022-9). FIG. 30F shows that the spleen, liver and lymph node(s) of mice treated with P2GA/P1A3 or P2C4/P1A10 were found to demonstrate higher CD8/Treg ratios compared to the same organs from mice treated with IL-2. This shows that P2C4/P1A3 and P2C4/P1A10 preferentially expand CD& T cells over Tregs, compared to the effect seen with IL-2.

The numbers of CD3, CD4 and CD8 T cells from spleen, liver, tumour-draining lymph node and injection site were pooled to provide total numbers of cells. The results are shown in FIG. 30G. The total pooled CD8/Treg ratio is shown in FIG. 30H. The total CD8/Treg ratio of mice treated with P2C4/P1A3 or P2C4/P1A10 was found to be higher than the total CD8/Treg ratio from mice treated with isotype (Iso) or IL-2; *, p<0.05.

The numbers of EBV-BLCLs present in the spleen, liver and lymph node(s) at the end of the experiment were also analysed. The results are shown in FIG. 30I. Mice treated with P2C4/P1A3 and P2C4/P1A10 were found to have reduced numbers of EBV-BLCL tumour cells compared to mica treated with IL-2**, p<0.01; *, p<0.05.

Total organ tumour load in mice was calculated from the total numbers of CD19+ tumour cells found in spleen, liver, and tumour-draining lymph node. The results are shown in FIG. 30J. Mice treated with P2CA/P1A3 and P2C4/P1A10 were found to have lower total organ tumour burden as compared to mice treated with isotype control antibody. IL-2 or no VSTs; **, p<0.01.

Next, the cytolytic activity of the expanded CD8 T cells was assessed by identifying the total number of CD8 T cells from spleen, liver, tumour-draining lymph node and injection site secreting the effector molecules interferon-γ (IFN-γ), CD107a and perforin. The results are shown in FIG. 30K. Higher numbers of effector molecule-secreting CD8 T cells were found to be present following treatment with P2C4/P1A3 and P2C4/P1A10 compared to treatment with IL-2. *, P<0.05.

In conclusion, bispecific anti-IL-2R and -ye antibodies were shown to provide sustained expansion of COB T cells in vivo without accompanying increases in Tregs, leading to improved tumour control.

### Example 11: Analysis of the Effect of Anti-IL-2Rβ/γc Antibodies on Survival

A murine model of metastatic lymphoma is generated by intravenous injection of EBV-BLCLs to investigate the effect of bispecific agonist anti-IL-2R and -ye antibodies versus IL-2 on survival. Mice treated with anti-IL-2R and -ye antibodies are found to have improved survival as compared to mice not treated with anti-IL-2R and -ye antibodies.

1. An antigen-binding molecule, optionally isolated, which is capable of binding to common γ chain (CD132), wherein the antigen-binding molecule comprises:
(P1A10) a VH region incorporating the following CDRs:
HC-CDR1 having the amino acid sequence of SEQ ID NO: 196
HC-CDR2 having the amino acid sequence of SEQ ID NO: 204
HC-CDR3 having the amino acid sequence of SEQ ID NO: 212; and
a VL region incorporating the following CDRs:
LC-CDR1 having the amino acid sequence of SEQ ID NO: 227
LC-CDR2 having the amino acid sequence of SEQ ID NO: 238
LC-CDR3 having the amino acid sequence of SEQ ID NO: 248; or
(P1B6) a VH region incorporating the following CDRs:
HC-CDR1 having the amino acid sequence of SEQ ID NO: 108
HC-CDR2 having the amino acid sequence of SEQ ID NO: 120
HC-CDR3 having the amino acid sequence of SEQ ID NO: 213; and
a VL region incorporating the following CDRs:
LC-CDR1 having the amino acid sequence of SEQ ID NO: 151
LC-CDR2 having the amino acid sequence of SEQ ID NO: 239
LC-CDR3 having the amino acid sequence of SEQ ID NO: 249; or
(P1C10) a VH region incorporating the following CDRs:
HC-CDR1 having the amino acid sequence of SEQ ID NO: 112
HC-CDR2 having the amino acid sequence of SEQ ID NO: 124
HC-CDR3 having the amino acid sequence of SEQ ID NO: 214; and
a VL region incorporating the following CDRs:
LC-CDR1 having the amino acid sequence of SEQ ID NO: 228
LC-CDR2 having the amino acid sequence of SEQ ID NO: 240
LC-CDR3 having the amino acid sequence of SEQ ID NO: 250; or
(P1D7) a VH region incorporating the following CDRs:
HC-CDR1 having the amino acid sequence of SEQ ID NO: 197
HC-CDR2 having the amino acid sequence of SEQ ID NO: 206
HC-CDR3 having the amino acid sequence of SEQ ID NO: 215; and
a VL region incorporating the following CDRs:
LC-CDR1 having the amino acid sequence of SEQ ID NO: 229
LC-CDR2 having the amino acid sequence of SEQ ID NO: 241
LC-CDR3 having the amino acid sequence of SEQ ID NO: 251; or
(P1E8) a VH region incorporating the following CDRs:
HC-CDR1 having the amino acid sequence of SEQ ID NO: 198
HC-CDR2 having the amino acid sequence of SEQ ID NO: 120
HC-CDR3 having the amino acid sequence of SEQ ID NO: 216; and
a VL region incorporating the following CDRs:
LC-CDR1 having the amino acid sequence of SEQ ID NO: 230
LC-CDR2 having the amino acid sequence of SEQ ID NO: 242
LC-CDR3 having the amino acid sequence of SEQ ID NO: 252; or
(P2B2) a VH region incorporating the following CDRs:
HC-CDR1 having the amino acid sequence of SEQ ID NO: 108
HC-CDR2 having the amino acid sequence of SEQ ID NO: 207
HC-CDR3 having the amino acid sequence of SEQ ID NO: 217; and
a VL region incorporating the following CDRs:
LC-CDR1 having the amino acid sequence of SEQ ID NO: 151
LC-CDR2 having the amino acid sequence of SEQ ID NO: 174
LC-CDR3 having the amino acid sequence of SEQ ID NO: 253; or
(P2B7) a VH region incorporating the following CDRs:
HC-CDR1 having the amino acid sequence of SEQ ID NO: 106
HC-CDR2 having the amino acid sequence of SEQ ID NO: 119
HC-CDR3 having the amino acid sequence of SEQ ID NO: 218; and
a VL region incorporating the following CDRs:
LC-CDR1 having the amino acid sequence of SEQ ID NO: 231
LC-CDR2 having the amino acid sequence of SEQ ID NO: 174
LC-CDR3 having the amino acid sequence of SEQ ID NO: 254; or
(P2D11) a VH region incorporating the following CDRs:
HC-CDR1 having the amino acid sequence of SEQ ID NO: 199
HC-CDR2 having the amino acid sequence of SEQ ID NO: 208
HC-CDR3 having the amino acid sequence of SEQ ID NO: 219; and
a VL region incorporating the following CDRs:
LC-CDR1 having the amino acid sequence of SEQ ID NO: 232
LC-CDR2 having the amino acid sequence of SEQ ID NO: 243
LC-CDR3 having the amino acid sequence of SEQ ID NO: 255; or
(P2F10) a VH region incorporating the following CDRs:
HC-CDR1 having the amino acid sequence of SEQ ID NO: 200
HC-CDR2 having the amino acid sequence of SEQ ID NO: 209
HC-CDR3 having the amino acid sequence of SEQ ID NO: 220; and
a VL region incorporating the following CDRs:
LC-CDR1 having the amino acid sequence of SEQ ID NO: 233
LC-CDR2 having the amino acid sequence of SEQ ID NO: 244
LC-CDR3 having the amino acid sequence of SEQ ID NO: 256; or
(P2H4) a VH region incorporating the following CDRs:
HC-CDR1 having the amino acid sequence of SEQ ID NO: 108
HC-CDR2 having the amino acid sequence of SEQ ID NO: 120
HC-CDR3 having the amino acid sequence of SEQ ID NO: 221; and
a VL region incorporating the following CDRs:
LC-CDR1 having the amino acid sequence of SEQ ID NO: 234
LC-CDR2 having the amino acid sequence of SEQ ID NO: 174
LC-CDR3 having the amino acid sequence of SEQ ID NO: 257; or
(P2D3) a VH region incorporating the following CDRs:
HC-CDR1 having the amino acid sequence of SEQ ID NO: 201
HC-CDR2 having the amino acid sequence of SEQ ID NO: 119
HC-CDR3 having the amino acid sequence of SEQ ID NO: 222; and
a VL region incorporating the following CDRs:
LC-CDR1 having the amino acid sequence of SEQ ID NO: 151
LC-CDR2 having the amino acid sequence of SEQ ID NO: 174
LC-CDR3 having the amino acid sequence of SEQ ID NO: 189; or
(P1G4) a VH region incorporating the following CDRs:
HC-CDR1 having the amino acid sequence of SEQ ID NO: 106
HC-CDR2 having the amino acid sequence of SEQ ID NO: 119
HC-CDR3 having the amino acid sequence of SEQ ID NO: 223; and
a VL region incorporating the following CDRs:
LC-CDR1 having the amino acid sequence of SEQ ID NO: 151
LC-CDR2 having the amino acid sequence of SEQ ID NO: 174
LC-CDR3 having the amino acid sequence of SEQ ID NO: 258; or
(P1B12) a VH region incorporating the following CDRs:
HC-CDR1 having the amino acid sequence of SEQ ID NO: 106
HC-CDR2 having the amino acid sequence of SEQ ID NO: 119
HC-CDR3 having the amino acid sequence of SEQ ID NO: 224; and
a VL region incorporating the following CDRs:
LC-CDR1 having the amino acid sequence of SEQ ID NO: 235
LC-CDR2 having the amino acid sequence of SEQ ID NO: 174
LC-CDR3 having the amino acid sequence of SEQ ID NO: 189; or
(P1C7) a VH region incorporating the following CDRs:
HC-CDR1 having the amino acid sequence of SEQ ID NO: 106
HC-CDR2 having the amino acid sequence of SEQ ID NO: 119
HC-CDR3 having the amino acid sequence of SEQ ID NO: 225; and
a VL region incorporating the following CDRs:
LC-CDR1 having the amino acid sequence of SEQ ID NO: 151
LC-CDR2 having the amino acid sequence of SEQ ID NO: 245
LC-CDR3 having the amino acid sequence of SEQ ID NO: 189.

2. The antigen-binding molecule according to claim 1, wherein the antigen-binding molecule comprises:
(P1A10) a VH region comprising an amino acid sequence having at least 85% sequence identity to SEQ ID NO: 71; and
a VL region comprising an amino acid sequence having at least 85% sequence identity to SEQ ID NO: 89; or
(P1B6) a VH region comprising an amino acid sequence having at least 85% sequence identity to SEQ ID NO: 72; and
a VL region comprising an amino acid sequence having at least 85% sequence identity to SEQ ID NO: 90; or
(P1C10) a VH region comprising an amino acid sequence having at least 85% sequence identity to SEQ ID NO: 73; and
a VL region comprising an amino acid sequence having at least 85% sequence identity to SEQ ID NO: 91; or
(P1D7) a VH region comprising an amino acid sequence having at least 85% sequence identity to SEQ ID NO: 74; and
a VL region comprising an amino acid sequence having at least 85% sequence identity to SEQ ID NO: 92; or
(P1E8) a VH region comprising an amino acid sequence having at least 85% sequence identity to SEQ ID NO: 75; and
a VL region comprising an amino acid sequence having at least 85% sequence identity to SEQ ID NO: 93; or
(P2B2) a VH region comprising an amino acid sequence having at least 85% sequence identity to SEQ ID NO: 76; and
a VL region comprising an amino acid sequence having at least 85% sequence identity to SEQ ID NO: 94; or
(P2B7) a VH region comprising an amino acid sequence having at least 85% sequence identity to SEQ ID NO: 77; and
a VL region comprising an amino acid sequence having at least 85% sequence identity to SEQ ID NO: 95; or
(P2D11) a VH region comprising an amino acid sequence having at least 85% sequence identity to SEQ ID NO: 78; and
a VL region comprising an amino acid sequence having at least 85% sequence identity to SEQ ID NO: 96; or
(P2F10) a VH region comprising an amino acid sequence having at least 85% sequence identity to SEQ ID NO: 79; and
a VL region comprising an amino acid sequence having at least 85% sequence identity to SEQ ID NO: 97; or
(P2H4) a VH region comprising an amino acid sequence having at least 85% sequence identity to SEQ ID NO: 80; and
a VL region comprising an amino acid sequence having at least 85% sequence identity to SEQ ID NO: 98; or
(P2D3) a VH region comprising an amino acid sequence having at least 85% sequence identity to SEQ ID NO: 81; and
a VL region comprising an amino acid sequence having at least 85% sequence identity to SEQ ID NO: 99; or
(P1G4) a VH region comprising an amino acid sequence having at least 85% sequence identity to SEQ ID NO: 82; and
a VL region comprising an amino acid sequence having at least 85% sequence identity to SEQ ID NO: 100; or
(P1B12) a VH region comprising an amino acid sequence having at least 85% sequence identity to SEQ ID NO: 83; and
a VL region comprising an amino acid sequence having at least 85% sequence identity to SEQ ID NO: 101; or
(P1C7) a VH region comprising an amino acid sequence having at least 85% sequence identity to SEQ ID NO: 84; and
a VL region comprising an amino acid sequence having at least 85% sequence identity to SEQ ID NO: 102.

3. The antigen-binding molecule according to claim 1, wherein the antigen binding molecule is capable of binding to CD122 and CD132.

4. The antigen-binding molecule according to claim 1, which is an IL-2 receptor agonist.

5. The antigen-binding molecule according to claim 1, which is capable of reducing expression of PD-1 by T cells.

6. A nucleic acid encoding an antigen-binding molecule, optionally isolated, which is capable of binding to common γ chain (CD132), wherein the antigen-binding molecule comprises:
(P1A10) a VH region incorporating the following CDRs:
HC-CDR1 having the amino acid sequence of SEQ ID NO: 196
HC-CDR2 having the amino acid sequence of SEQ ID NO: 204
HC-CDR3 having the amino acid sequence of SEQ ID NO: 212; and
a VL region incorporating the following CDRs:
LC-CDR1 having the amino acid sequence of SEQ ID NO: 227
LC-CDR2 having the amino acid sequence of SEQ ID NO: 238
LC-CDR3 having the amino acid sequence of SEQ ID NO: 248; or
(P1B6) a VH region incorporating the following CDRs:
HC-CDR1 having the amino acid sequence of SEQ ID NO: 108
HC-CDR2 having the amino acid sequence of SEQ ID NO: 120
HC-CDR3 having the amino acid sequence of SEQ ID NO: 213; and
a VL region incorporating the following CDRs:
LC-CDR1 having the amino acid sequence of SEQ ID NO: 151
LC-CDR2 having the amino acid sequence of SEQ ID NO: 239
LC-CDR3 having the amino acid sequence of SEQ ID NO: 249; or
(P1C10) a VH region incorporating the following CDRs:
HC-CDR1 having the amino acid sequence of SEQ ID NO: 112
HC-CDR2 having the amino acid sequence of SEQ ID NO: 124
HC-CDR3 having the amino acid sequence of SEQ ID NO: 214; and
a VL region incorporating the following CDRs:
LC-CDR1 having the amino acid sequence of SEQ ID NO: 228
LC-CDR2 having the amino acid sequence of SEQ ID NO: 240
LC-CDR3 having the amino acid sequence of SEQ ID NO: 250; or
(P1D7) a VH region incorporating the following CDRs:
HC-CDR1 having the amino acid sequence of SEQ ID NO: 197
HC-CDR2 having the amino acid sequence of SEQ ID NO: 206
HC-CDR3 having the amino acid sequence of SEQ ID NO: 215; and
a VL region incorporating the following CDRs:
LC-CDR1 having the amino acid sequence of SEQ ID NO: 229
LC-CDR2 having the amino acid sequence of SEQ ID NO: 241
LC-CDR3 having the amino acid sequence of SEQ ID NO: 251; or
(P1E8) a VH region incorporating the following CDRs:
HC-CDR1 having the amino acid sequence of SEQ ID NO: 198
HC-CDR2 having the amino acid sequence of SEQ ID NO: 120
HC-CDR3 having the amino acid sequence of SEQ ID NO: 216; and
a VL region incorporating the following CDRs:
LC-CDR1 having the amino acid sequence of SEQ ID NO: 230
LC-CDR2 having the amino acid sequence of SEQ ID NO: 242
LC-CDR3 having the amino acid sequence of SEQ ID NO: 252; or
(P2B2) a VH region incorporating the following CDRs:
HC-CDR1 having the amino acid sequence of SEQ ID NO: 108
HC-CDR2 having the amino acid sequence of SEQ ID NO: 207
HC-CDR3 having the amino acid sequence of SEQ ID NO: 217; and
a VL region incorporating the following CDRs:
LC-CDR1 having the amino acid sequence of SEQ ID NO: 151
LC-CDR2 having the amino acid sequence of SEQ ID NO: 174
LC-CDR3 having the amino acid sequence of SEQ ID NO: 253; or
(P2B7) a VH region incorporating the following CDRs:
HC-CDR1 having the amino acid sequence of SEQ ID NO: 106
HC-CDR2 having the amino acid sequence of SEQ ID NO: 119
HC-CDR3 having the amino acid sequence of SEQ ID NO: 218; and
a VL region incorporating the following CDRs:
LC-CDR1 having the amino acid sequence of SEQ ID NO: 231
LC-CDR2 having the amino acid sequence of SEQ ID NO: 174
LC-CDR3 having the amino acid sequence of SEQ ID NO: 254; or
(P2D11) a VH region incorporating the following CDRs:
HC-CDR1 having the amino acid sequence of SEQ ID NO: 199
HC-CDR2 having the amino acid sequence of SEQ ID NO: 208
HC-CDR3 having the amino acid sequence of SEQ ID NO: 219; and
a VL region incorporating the following CDRs:
LC-CDR1 having the amino acid sequence of SEQ ID NO: 232
LC-CDR2 having the amino acid sequence of SEQ ID NO: 243
LC-CDR3 having the amino acid sequence of SEQ ID NO: 255; or
(P2F10) a VH region incorporating the following CDRs:
HC-CDR1 having the amino acid sequence of SEQ ID NO: 200
HC-CDR2 having the amino acid sequence of SEQ ID NO: 209
HC-CDR3 having the amino acid sequence of SEQ ID NO: 220; and
a VL region incorporating the following CDRs:
LC-CDR1 having the amino acid sequence of SEQ ID NO: 233
LC-CDR2 having the amino acid sequence of SEQ ID NO: 244
LC-CDR3 having the amino acid sequence of SEQ ID NO: 256; or
(P2H4) a VH region incorporating the following CDRs:
HC-CDR1 having the amino acid sequence of SEQ ID NO: 108
HC-CDR2 having the amino acid sequence of SEQ ID NO: 120
HC-CDR3 having the amino acid sequence of SEQ ID NO: 221; and
a VL region incorporating the following CDRs:
LC-CDR1 having the amino acid sequence of SEQ ID NO: 234
LC-CDR2 having the amino acid sequence of SEQ ID NO: 174
LC-CDR3 having the amino acid sequence of SEQ ID NO: 257; or
(P2D3) a VH region incorporating the following CDRs:
HC-CDR1 having the amino acid sequence of SEQ ID NO: 201
HC-CDR2 having the amino acid sequence of SEQ ID NO: 119
HC-CDR3 having the amino acid sequence of SEQ ID NO: 222; and
a VL region incorporating the following CDRs:
LC-CDR1 having the amino acid sequence of SEQ ID NO: 151
LC-CDR2 having the amino acid sequence of SEQ ID NO: 174
LC-CDR3 having the amino acid sequence of SEQ ID NO: 189; or
(P1G4) a VH region incorporating the following CDRs:
HC-CDR1 having the amino acid sequence of SEQ ID NO: 106
HC-CDR2 having the amino acid sequence of SEQ ID NO: 119
HC-CDR3 having the amino acid sequence of SEQ ID NO: 223; and
a VL region incorporating the following CDRs:
LC-CDR1 having the amino acid sequence of SEQ ID NO: 151
LC-CDR2 having the amino acid sequence of SEQ ID NO: 174
LC-CDR3 having the amino acid sequence of SEQ ID NO: 258; or
(P1B12) a VH region incorporating the following CDRs:
HC-CDR1 having the amino acid sequence of SEQ ID NO: 106
HC-CDR2 having the amino acid sequence of SEQ ID NO: 119
HC-CDR3 having the amino acid sequence of SEQ ID NO: 224; and
a VL region incorporating the following CDRs:
LC-CDR1 having the amino acid sequence of SEQ ID NO: 235
LC-CDR2 having the amino acid sequence of SEQ ID NO: 174
LC-CDR3 having the amino acid sequence of SEQ ID NO: 189; or
(P1C7) a VH region incorporating the following CDRs:
HC-CDR1 having the amino acid sequence of SEQ ID NO: 106
HC-CDR2 having the amino acid sequence of SEQ ID NO: 119
HC-CDR3 having the amino acid sequence of SEQ ID NO: 225; and
a VL region incorporating the following CDRs:
LC-CDR1 having the amino acid sequence of SEQ ID NO: 151
LC-CDR2 having the amino acid sequence of SEQ ID NO: 245
LC-CDR3 having the amino acid sequence of SEQ ID NO: 189.

7. The nucleic acid encoding an antigen-binding molecule according to claim 6, wherein the antigen-binding molecule comprises:
(P1A10) a VH region comprising an amino acid sequence having at least 85% sequence identity to SEQ ID NO: 71; and
a VL region comprising an amino acid sequence having at least 85% sequence identity to SEQ ID NO: 89; or
(P1B6) a VH region comprising an amino acid sequence having at least 85% sequence identity to SEQ ID NO: 72; and
a VL region comprising an amino acid sequence having at least 85% sequence identity to SEQ ID NO: 90; or
(P1C10) a VH region comprising an amino acid sequence having at least 85% sequence identity to SEQ ID NO: 73; and
a VL region comprising an amino acid sequence having at least 85% sequence identity to SEQ ID NO: 91; or
(P1D7) a VH region comprising an amino acid sequence having at least 85% sequence identity to SEQ ID NO: 74; and
a VL region comprising an amino acid sequence having at least 85% sequence identity to SEQ ID NO: 92; or
(P1E8) a VH region comprising an amino acid sequence having at least 85% sequence identity to SEQ ID NO: 75; and
a VL region comprising an amino acid sequence having at least 85% sequence identity to SEQ ID NO: 93; or
(P2B2) a VH region comprising an amino acid sequence having at least 85% sequence identity to SEQ ID NO: 76; and
a VL region comprising an amino acid sequence having at least 85% sequence identity to SEQ ID NO: 94; or
(P2B7) a VH region comprising an amino acid sequence having at least 85% sequence identity to SEQ ID NO: 77; and
a VL region comprising an amino acid sequence having at least 85% sequence identity to SEQ ID NO: 95; or
(P2D11) a VH region comprising an amino acid sequence having at least 85% sequence identity to SEQ ID NO: 78; and
a VL region comprising an amino acid sequence having at least 85% sequence identity to SEQ ID NO: 96; or
(P2F10) a VH region comprising an amino acid sequence having at least 85% sequence identity to SEQ ID NO: 79; and
a VL region comprising an amino acid sequence having at least 85% sequence identity to SEQ ID NO: 97; or
(P2H4) a VH region comprising an amino acid sequence having at least 85% sequence identity to SEQ ID NO: 80; and
a VL region comprising an amino acid sequence having at least 85% sequence identity to SEQ ID NO: 98; or
(P2D3) a VH region comprising an amino acid sequence having at least 85% sequence identity to SEQ ID NO: 81; and
a VL region comprising an amino acid sequence having at least 85% sequence identity to SEQ ID NO: 99; or
(P1G4) a VH region comprising an amino acid sequence having at least 85% sequence identity to SEQ ID NO: 82; and
a VL region comprising an amino acid sequence having at least 85% sequence identity to SEQ ID NO: 100; or
(P1B12) a VH region comprising an amino acid sequence having at least 85% sequence identity to SEQ ID NO: 83; and
a VL region comprising an amino acid sequence having at least 85% sequence identity to SEQ ID NO: 101; or
(P1C7) a VH region comprising an amino acid sequence having at least 85% sequence identity to SEQ ID NO: 84; and
a VL region comprising an amino acid sequence having at least 85% sequence identity to SEQ ID NO: 102.

8. The nucleic acid encoding an antigen-binding molecule according to claim 6, wherein the antigen binding molecule is capable of binding to CD122 and CD132.

9. The nucleic acid encoding an antigen-binding molecule according to claim 6, which is an IL-2 receptor agonist.

10. The nucleic acid encoding an antigen-binding molecule according to claim 6, which is capable of reducing expression of PD-1 by T cells.

11. A method of treating or preventing a T cell dysfunctional disorder, a cancer, or an infectious disease, comprising administering to a subject a therapeutically or prophylactically effective amount of an antigen-binding molecule, wherein the antigen-binding molecule comprises:
(P1A10) a VH region incorporating the following CDRs:
HC-CDR1 having the amino acid sequence of SEQ ID NO: 196
HC-CDR2 having the amino acid sequence of SEQ ID NO: 204
HC-CDR3 having the amino acid sequence of SEQ ID NO: 212; and
a VL region incorporating the following CDRs:
LC-CDR1 having the amino acid sequence of SEQ ID NO: 227
LC-CDR2 having the amino acid sequence of SEQ ID NO: 238
LC-CDR3 having the amino acid sequence of SEQ ID NO: 248; or
(P1B6) a VH region incorporating the following CDRs:
HC-CDR1 having the amino acid sequence of SEQ ID NO: 108
HC-CDR2 having the amino acid sequence of SEQ ID NO: 120
HC-CDR3 having the amino acid sequence of SEQ ID NO: 213; and
a VL region incorporating the following CDRs:
LC-CDR1 having the amino acid sequence of SEQ ID NO: 151
LC-CDR2 having the amino acid sequence of SEQ ID NO: 239
LC-CDR3 having the amino acid sequence of SEQ ID NO: 249; or
(P1C10) a VH region incorporating the following CDRs:
HC-CDR1 having the amino acid sequence of SEQ ID NO: 112
HC-CDR2 having the amino acid sequence of SEQ ID NO: 124
HC-CDR3 having the amino acid sequence of SEQ ID NO: 214; and
a VL region incorporating the following CDRs:
LC-CDR1 having the amino acid sequence of SEQ ID NO: 228
LC-CDR2 having the amino acid sequence of SEQ ID NO: 240
LC-CDR3 having the amino acid sequence of SEQ ID NO: 250; or
(P1D7) a VH region incorporating the following CDRs:
HC-CDR1 having the amino acid sequence of SEQ ID NO: 197
HC-CDR2 having the amino acid sequence of SEQ ID NO: 206
HC-CDR3 having the amino acid sequence of SEQ ID NO: 215; and
a VL region incorporating the following CDRs:
LC-CDR1 having the amino acid sequence of SEQ ID NO: 229
LC-CDR2 having the amino acid sequence of SEQ ID NO: 241
LC-CDR3 having the amino acid sequence of SEQ ID NO: 251; or
(P1E8) a VH region incorporating the following CDRs:
HC-CDR1 having the amino acid sequence of SEQ ID NO: 198
HC-CDR2 having the amino acid sequence of SEQ ID NO: 120
HC-CDR3 having the amino acid sequence of SEQ ID NO: 216; and
a VL region incorporating the following CDRs:
LC-CDR1 having the amino acid sequence of SEQ ID NO: 230
LC-CDR2 having the amino acid sequence of SEQ ID NO: 242
LC-CDR3 having the amino acid sequence of SEQ ID NO: 252; or
(P2B2) a VH region incorporating the following CDRs:
HC-CDR1 having the amino acid sequence of SEQ ID NO: 108
HC-CDR2 having the amino acid sequence of SEQ ID NO: 207
HC-CDR3 having the amino acid sequence of SEQ ID NO: 217; and
a VL region incorporating the following CDRs:
LC-CDR1 having the amino acid sequence of SEQ ID NO: 151
LC-CDR2 having the amino acid sequence of SEQ ID NO: 174
LC-CDR3 having the amino acid sequence of SEQ ID NO: 253; or
(P2B7) a VH region incorporating the following CDRs:
HC-CDR1 having the amino acid sequence of SEQ ID NO: 106
HC-CDR2 having the amino acid sequence of SEQ ID NO: 119
HC-CDR3 having the amino acid sequence of SEQ ID NO: 218; and
a VL region incorporating the following CDRs:
LC-CDR1 having the amino acid sequence of SEQ ID NO: 231
LC-CDR2 having the amino acid sequence of SEQ ID NO: 174
LC-CDR3 having the amino acid sequence of SEQ ID NO: 254; or
(P2D11) a VH region incorporating the following CDRs:
HC-CDR1 having the amino acid sequence of SEQ ID NO: 199
HC-CDR2 having the amino acid sequence of SEQ ID NO: 208
HC-CDR3 having the amino acid sequence of SEQ ID NO: 219; and
a VL region incorporating the following CDRs:
LC-CDR1 having the amino acid sequence of SEQ ID NO: 232
LC-CDR2 having the amino acid sequence of SEQ ID NO: 243
LC-CDR3 having the amino acid sequence of SEQ ID NO: 255; or
(P2F10) a VH region incorporating the following CDRs:
HC-CDR1 having the amino acid sequence of SEQ ID NO: 200
HC-CDR2 having the amino acid sequence of SEQ ID NO: 209
HC-CDR3 having the amino acid sequence of SEQ ID NO: 220; and
a VL region incorporating the following CDRs:
LC-CDR1 having the amino acid sequence of SEQ ID NO: 233
LC-CDR2 having the amino acid sequence of SEQ ID NO: 244
LC-CDR3 having the amino acid sequence of SEQ ID NO: 256; or
(P2H4) a VH region incorporating the following CDRs:
HC-CDR1 having the amino acid sequence of SEQ ID NO: 108
HC-CDR2 having the amino acid sequence of SEQ ID NO: 120
HC-CDR3 having the amino acid sequence of SEQ ID NO: 221; and
a VL region incorporating the following CDRs:
LC-CDR1 having the amino acid sequence of SEQ ID NO: 234
LC-CDR2 having the amino acid sequence of SEQ ID NO: 174
LC-CDR3 having the amino acid sequence of SEQ ID NO: 257; or
(P2D3) a VH region incorporating the following CDRs:
HC-CDR1 having the amino acid sequence of SEQ ID NO: 201
HC-CDR2 having the amino acid sequence of SEQ ID NO: 119
HC-CDR3 having the amino acid sequence of SEQ ID NO: 222; and
a VL region incorporating the following CDRs:
LC-CDR1 having the amino acid sequence of SEQ ID NO: 151
LC-CDR2 having the amino acid sequence of SEQ ID NO: 174
LC-CDR3 having the amino acid sequence of SEQ ID NO: 189; or
(P1G4) a VH region incorporating the following CDRs:
HC-CDR1 having the amino acid sequence of SEQ ID NO: 106
HC-CDR2 having the amino acid sequence of SEQ ID NO: 119
HC-CDR3 having the amino acid sequence of SEQ ID NO: 223; and
a VL region incorporating the following CDRs:
LC-CDR1 having the amino acid sequence of SEQ ID NO: 151
LC-CDR2 having the amino acid sequence of SEQ ID NO: 174
LC-CDR3 having the amino acid sequence of SEQ ID NO: 258; or
(P1B12) a VH region incorporating the following CDRs:
HC-CDR1 having the amino acid sequence of SEQ ID NO: 106
HC-CDR2 having the amino acid sequence of SEQ ID NO: 119
HC-CDR3 having the amino acid sequence of SEQ ID NO: 224; and
a VL region incorporating the following CDRs:
LC-CDR1 having the amino acid sequence of SEQ ID NO: 235
LC-CDR2 having the amino acid sequence of SEQ ID NO: 174
LC-CDR3 having the amino acid sequence of SEQ ID NO: 189; or
(P1C7) a VH region incorporating the following CDRs:
HC-CDR1 having the amino acid sequence of SEQ ID NO: 106
HC-CDR2 having the amino acid sequence of SEQ ID NO: 119
HC-CDR3 having the amino acid sequence of SEQ ID NO: 225; and
a VL region incorporating the following CDRs:
LC-CDR1 having the amino acid sequence of SEQ ID NO: 151
LC-CDR2 having the amino acid sequence of SEQ ID NO: 245
LC-CDR3 having the amino acid sequence of SEQ ID NO: 189.

12. The method according to claim 11, wherein the antigen-binding molecule comprises:
(P1A10) a VH region comprising an amino acid sequence having at least 85% sequence identity to SEQ ID NO: 71; and
a VL region comprising an amino acid sequence having at least 85% sequence identity to SEQ ID NO: 89; or
(P1B6) a VH region comprising an amino acid sequence having at least 85% sequence identity to SEQ ID NO: 72; and
a VL region comprising an amino acid sequence having at least 85% sequence identity to SEQ ID NO: 90; or
(P1C10) a VH region comprising an amino acid sequence having at least 85% sequence identity to SEQ ID NO: 73; and
a VL region comprising an amino acid sequence having at least 85% sequence identity to SEQ ID NO: 91; or
(P1D7) a VH region comprising an amino acid sequence having at least 85% sequence identity to SEQ ID NO: 74; and
a VL region comprising an amino acid sequence having at least 85% sequence identity to SEQ ID NO: 92; or
(P1E8) a VH region comprising an amino acid sequence having at least 85% sequence identity to SEQ ID NO: 75; and
a VL region comprising an amino acid sequence having at least 85% sequence identity to SEQ ID NO: 93; or
(P2B2) a VH region comprising an amino acid sequence having at least 85% sequence identity to SEQ ID NO: 76; and
a VL region comprising an amino acid sequence having at least 85% sequence identity to SEQ ID NO: 94; or
(P2B7) a VH region comprising an amino acid sequence having at least 85% sequence identity to SEQ ID NO: 77; and
a VL region comprising an amino acid sequence having at least 85% sequence identity to SEQ ID NO: 95; or
(P2D11) a VH region comprising an amino acid sequence having at least 85% sequence identity to SEQ ID NO: 78; and
a VL region comprising an amino acid sequence having at least 85% sequence identity to SEQ ID NO: 96; or
(P2F10) a VH region comprising an amino acid sequence having at least 85% sequence identity to SEQ ID NO: 79; and
a VL region comprising an amino acid sequence having at least 85% sequence identity to SEQ ID NO: 97; or
(P2H4) a VH region comprising an amino acid sequence having at least 85% sequence identity to SEQ ID NO: 80; and
a VL region comprising an amino acid sequence having at least 85% sequence identity to SEQ ID NO: 98; or
(P2D3) a VH region comprising an amino acid sequence having at least 85% sequence identity to SEQ ID NO: 81; and
a VL region comprising an amino acid sequence having at least 85% sequence identity to SEQ ID NO: 99; or
(P1G4) a VH region comprising an amino acid sequence having at least 85% sequence identity to SEQ ID NO: 82; and
a VL region comprising an amino acid sequence having at least 85% sequence identity to SEQ ID NO: 100; or
(P1B12) a VH region comprising an amino acid sequence having at least 85% sequence identity to SEQ ID NO: 83; and
a VL region comprising an amino acid sequence having at least 85% sequence identity to SEQ ID NO: 101; or
(P1C7) a VH region comprising an amino acid sequence having at least 85% sequence identity to SEQ ID NO: 84; and
a VL region comprising an amino acid sequence having at least 85% sequence identity to SEQ ID NO: 102.

13. The method according to claim 11, wherein the antigen binding molecule is capable of binding to CD122 and CD132.

14. The method according to claim 11, wherein the cancer is selected from the group consisting of: colon cancer, colon carcinoma, colorectal cancer, nasopharyngeal carcinoma, cervical carcinoma, oropharyngeal carcinoma, gastric carcinoma, hepatocellular carcinoma, head and neck cancer, head and neck squamous cell carcinoma (HNSCC), oral cancer, laryngeal cancer, prostate cancer, lung cancer, small cell lung cancer, non-small cell lung cancer, bladder cancer, urothelial carcinoma, melanoma, advanced melanoma, renal cell carcinoma, ovarian cancer or mesothelioma.

15. The method according to claim 11, wherein the antigen binding molecule is administered in combination with a therapeutically effective amount of an agent capable of inhibiting signalling mediated by an immune checkpoint protein.

16. The method according to claim 15, wherein the immune checkpoint protein is PD-1, CTLA-4, LAG-3, TIM-3, VISTA, TIGIT or BTLA.

17. The method according to claim 11, wherein the infectious disease causes an inability to mount an effective immune response against an infection, and wherein the infection is a chronic and/or latent infection.

18. The method according to claim 11, wherein the infectious disease is caused by a bacterial, fungal, parasitic, or viral infection.

19. The method according to claim 11, wherein the infectious disease caused by a bacterial infection is selected from: Bacillus spp., Bordetella pertussis, Clostridium spp., Corynebacterium spp., Vibrio chloerae, Staphylococcus spp., Streptococcus spp. Escherichia, Klebsiella, Proteus, Yersinia, Erwina, Salmonella, Listeria sp, Helicobacter pylori, mycobacteria (e.g. Mycobacterium tuberculosis) and Pseudomonas aeruginosa;
wherein the infectious disease caused by a fungal infection is selected from: Alternaria sp, Aspergillus sp, Candida sp and Histoplasma sp;
wherein the infectious disease caused by a parasitic infection is selected from: diseases caused by Plasmodium species (e.g. Plasmodium falciparum, Plasmodium yoeli, Plasmodium ovale, Plasmodium vivax, or Plasmodium chabaudi chabaudi), malaria, leishmaniasis, and toxoplasmosis; or
wherein the infectious disease caused by a viral infection is selected from by influenza virus, measles virus, hepatitis B virus (HBV), hepatitis C virus (HCV), human immunodeficiency virus (HIV), lymphocytic choriomeningitis virus (LCMV), Herpes simplex virus and human papilloma virus (HPV).

20. The method according to claim 11, wherein the T cell dysfunctional disorder comprises T cell exhaustion or T cell anergy.
